SEQUENCE LISTING (50 sequences)

SEQ ID NO: 1 - protein, 117 aa, Artificial Sequence
  Antibody sequence
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 2 - protein, 5 aa, Artificial Sequence
  Antibody sequence
NAWMS

SEQ ID NO: 3 - protein, 17 aa, Artificial Sequence
  Antibody sequence
YISSSGSTIYYADSVKG

SEQ ID NO: 4 - protein, 8 aa, Artificial Sequence
  Antibody sequence
EGLWAFDY

SEQ ID NO: 5 - protein, 111 aa, Artificial Sequence
  Antibody sequence
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVL

SEQ ID NO: 6 - protein, 14 aa, Artificial Sequence
  Antibody sequence
TGSSSNIGAGYVVH

SEQ ID NO: 7 - protein, 7 aa, Artificial Sequence
  Antibody sequence
DNNKRPS

SEQ ID NO: 8 - protein, 11 aa, Artificial Sequence
  Antibody sequence
AAWDDRLNGPV

SEQ ID NO: 9 - protein, 446 aa, Artificial Sequence
  Antibody sequence
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 217 aa, Artificial Sequence
  Antibody sequence
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 117 aa, Artificial Sequence
  Antibody sequence
EVQLLESGGGLVQPGGSLRLSCAASGFTFSSAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 12 - protein, 5 aa, Artificial Sequence
  Antibody sequence
SAWMS

SEQ ID NO: 13 - protein, 17 aa, Artificial Sequence
  Antibody sequence
YISSSGSTIYYADSVKG

SEQ ID NO: 14 - protein, 8 aa, Artificial Sequence
  Antibody sequence
EGLWAFDY

SEQ ID NO: 15 - protein, 111 aa, Artificial Sequence
  Antibody sequence
QSVLTQPPSVSGAPGQRVTISCTGSSSNIGAGYVVHWYQQLPGTAPKLLIYDNNKRPSGVPDRFSGSKSGTSASLAISGLRSEDEADYYCAAYDDSLSGPVFGGGTKLTVL

SEQ ID NO: 16 - protein, 14 aa, Artificial Sequence
  Antibody sequence
TGSSSNIGAGYVVH

SEQ ID NO: 17 - protein, 7 aa, Artificial Sequence
  Antibody sequence
DNNKRPS

SEQ ID NO: 18 - protein, 11 aa, Artificial Sequence
  Antibody sequence
AAYDDSLSGPV

SEQ ID NO: 19 - protein, 446 aa, Artificial Sequence
  Antibody sequence
EVQLLESGGGLVQPGGSLRLSCAASGFTFSSAWMSWVRQAPGKGLEWVSYISSSGSTIYYADSVKGRFTISRDNSKNTLYLQMNSLRAEDTAVYYCAREGLWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 217 aa, Artificial Sequence
  Antibody sequence
QSVLTQPPSVSGAPGQRVTISCTGSSSNIGAGYVVHWYQQLPGTAPKLLIYDNNKRPSGVPDRFSGSKSGTSASLAISGLRSEDEADYYCAAYDDSLSGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 120 aa, Artificial Sequence [sequence omitted]

SEQ ID NO: 22 - protein, 5 aa, Artificial Sequence
  Antibody sequence
DTYIH

SEQ ID NO: 23 - protein, 17 aa, Artificial Sequence
  Antibody sequence
RIYPTNGYTRYADSVKG

SEQ ID NO: 24 - protein, 11 aa, Artificial Sequence
  Antibody sequence
WGGDGFYAMDY

SEQ ID NO: 25 - protein, 107 aa, Artificial Sequence [sequence omitted]

SEQ ID NO: 26 - protein, 11 aa, Artificial Sequence
  Antibody sequence
RASQDVNTAVA

SEQ ID NO: 27 - protein, 7 aa, Artificial Sequence
  Antibody sequence
SASFLYS

SEQ ID NO: 28 - protein, 9 aa, Artificial Sequence
  Antibody sequence
QQHYTTPPT

SEQ ID NO: 29 - protein, 450 aa, Artificial Sequence
  Antibody sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, Artificial Sequence
  Antibody sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 117 aa, Artificial Sequence
  Antibody sequence
EVQLLESGGGLVQPGGSLRLSCAASGFTFSSYAMSWVRQAPGKGLEWVSSISGSGGSTLYADSVKGRFTISRDNSKNTLYLQMNSLRAEDTAVYYCAKLTGTSFDYWGQGTLVTVSS

SEQ ID NO: 32 - protein, 5 aa, Artificial Sequence
  Antibody sequence
SYAMS

SEQ ID NO: 33 - protein, 17 aa, Artificial Sequence
  Antibody sequence
SISGSGGSTLYADSVKG

SEQ ID NO: 34 - protein, 8 aa, Artificial Sequence
  Antibody sequence
LTGTSFDY

SEQ ID NO: 35 - protein, 109 aa, Artificial Sequence
  Antibody sequence
QSVLTQPPSASGTPGQRVTISCSGSSSNIGSNPVNWYQQLPGTAPKLLIYSNNQRPSGVPDRFSGSKSGTSASLAITGLQSEDEADYYCQSFDSSLKKVFGGGTKLTVL

SEQ ID NO: 36 - protein, 13 aa, Artificial Sequence
  Antibody sequence
SGSSSNIGSNPVN

SEQ ID NO: 37 - protein, 7 aa, Artificial Sequence
  Antibody sequence
SNNQRPS

SEQ ID NO: 38 - protein, 10 aa, Artificial Sequence
  Antibody sequence
QSFDSSLKKV

SEQ ID NO: 39 - protein, 446 aa, Artificial Sequence
  Antibody sequence
EVQLLESGGGLVQPGGSLRLSCAASGFTFSSYAMSWVRQAPGKGLEWVSSISGSGGSTLYADSVKGRFTISRDNSKNTLYLQMNSLRAEDTAVYYCAKLTGTSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 215 aa, Artificial Sequence
  Antibody sequence
QSVLTQPPSASGTPGQRVTISCSGSSSNIGSNPVNWYQQLPGTAPKLLIYSNNQRPSGVPDRFSGSKSGTSASLAITGLQSEDEADYYCQSFDSSLKKVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 113 aa, Artificial Sequence
  Antibody sequence
EVQLVESGGGLIQPGGSLRLSCAASGFTFSTSGMHWFRQAPGKGLEWVAYISSSSGFVYADAVKGRFTISRDNSKNTLYLQMNSLRAEDTAVYYCARSEAAFWGQGTLVTVSS

SEQ ID NO: 42 - protein, 5 aa, Artificial Sequence
  Antibody sequence
TSGMH

SEQ ID NO: 43 - protein, 16 aa, Artificial Sequence
  Antibody sequence
YISSSSGFVYADAVKG

SEQ ID NO: 44 - protein, 5 aa, Artificial Sequence
  Antibody sequence
SEAAF

SEQ ID NO: 45 - protein, 112 aa, Artificial Sequence
  Antibody sequence
DIVMTQSPLSLPVTPGEPASISCRSQKSRLSRMGITPLNWYLQKPGQSPQLLIYRMSNLASGVPDRFSGSGSGTDFTLKISRVEAEDVGVYYCAQFLEYPPTFGQGTKLEIK

SEQ ID NO: 46 - protein, 16 aa, Artificial Sequence
  Antibody sequence
RSQKSRLSRMGITPLN

SEQ ID NO: 47 - protein, 7 aa, Artificial Sequence
  Antibody sequence
RMSNLAS

SEQ ID NO: 48 - protein, 9 aa, Artificial Sequence
  Antibody sequence
AQFLEYPPT

SEQ ID NO: 49 - protein, 442 aa, Artificial Sequence
  Antibody sequence
EVQLVESGGGLIQPGGSLRLSCAASGFTFSTSGMHWFRQAPGKGLEWVAYISSSSGFVYADAVKGRFTISRDNSKNTLYLQMNSLRAEDTAVYYCARSEAAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 219 aa, Artificial Sequence
  Antibody sequence
DIVMTQSPLSLPVTPGEPASISCRSQKSRLSRMGITPLNWYLQKPGQSPQLLIYRMSNLASGVPDRFSGSGSGTDFTLKISRVEAEDVGVYYCAQFLEYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0360531 A1
(43) Pub. Date: Nov. 19, 2020

(54) ANTIBODY DRUG CONJUGATES (ADCS) WITH NAMPT INHIBITORS

(71) Applicants: Bayer Aktiengesellschaft, Leverkusen (DE); Bayer Pharma Aktiengesellschaft, Berlin (DE)

(72) Inventors: Niels BÖHNKE, Berlin (DE); Markus BERGER, Berlin (DE); Anette SOMMER, Berlin (DE); Stefanie HAMMER, Berlin (DE); Amaury Ernesto FERNANDEZ-MONTALVAN, Le Pecq (FR); Beatrix STELTE-LUDWIG, Wülfrath (DE); Judith GÜNTHER, Berlin (DE); Christoph MAHLERT, Wuppertal (DE); Simone GREVEN, Dormagen (DE); Abul Basher SARKER, Leeds (GB); Michael TAIT, Macclesfield (GB)

(73) Assignees: Bayer Aktiengesellschaft, Leverkusen (DE); Bayer Pharma Aktiengesellschaft, Berlin (DE)

(21) Appl. No.: 16/966,409

(22) PCT Filed: Jan. 25, 2019
(86) PCT No.: PCT/EP2019/051888

§ 371 Date: Jul. 30, 2020

(30) Foreign Application Priority Data
Jan. 31, 2018 (EP) 18154417.2

(51) Int. Cl. A61K 47/68; C07D 471/04

(52) U.S. Cl. CPC A61K 47/6803; A61K 47/6849; A61K 45/06; C07D 471/04; A61K 47/6851

(57) ABSTRACT

Conjugate of a binder having formula: (A) wherein stands for a binder, Z′ stands for a linker and D stands for an active component of Formula (I): and its use as pharmaceuticals.

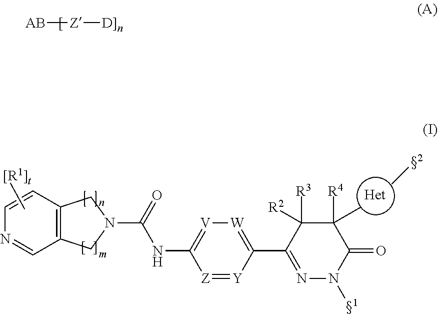

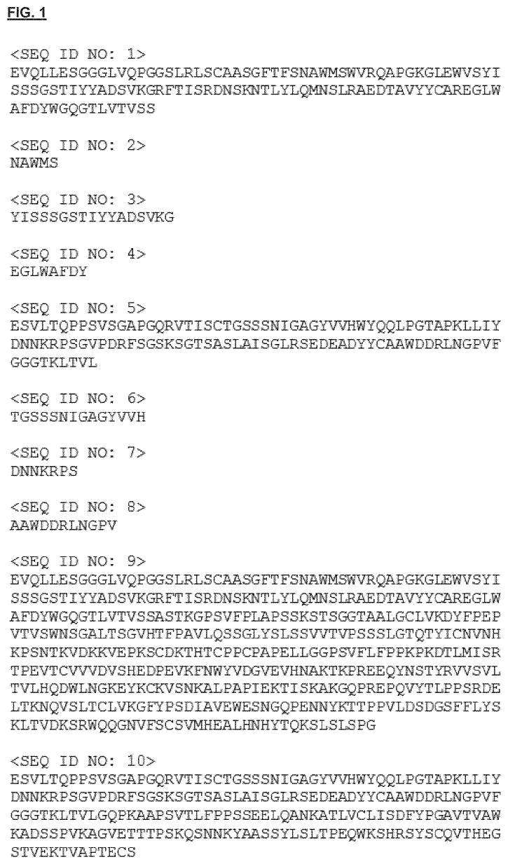

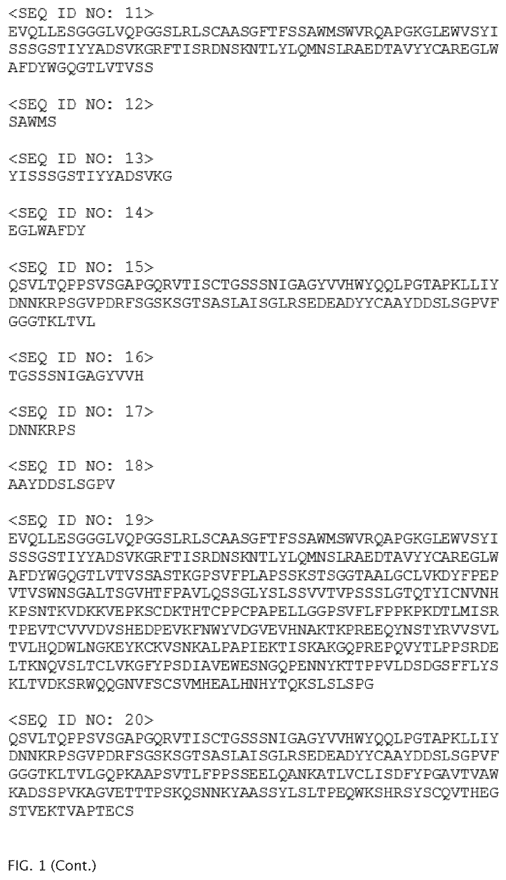

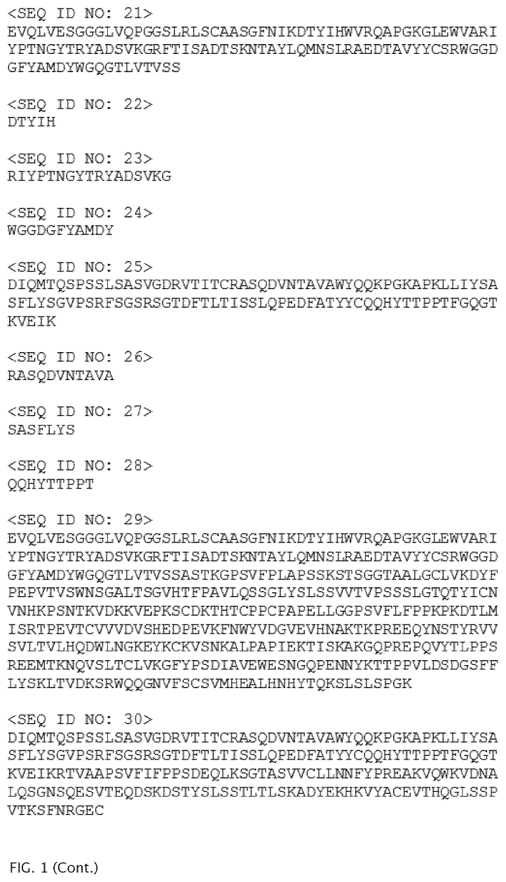

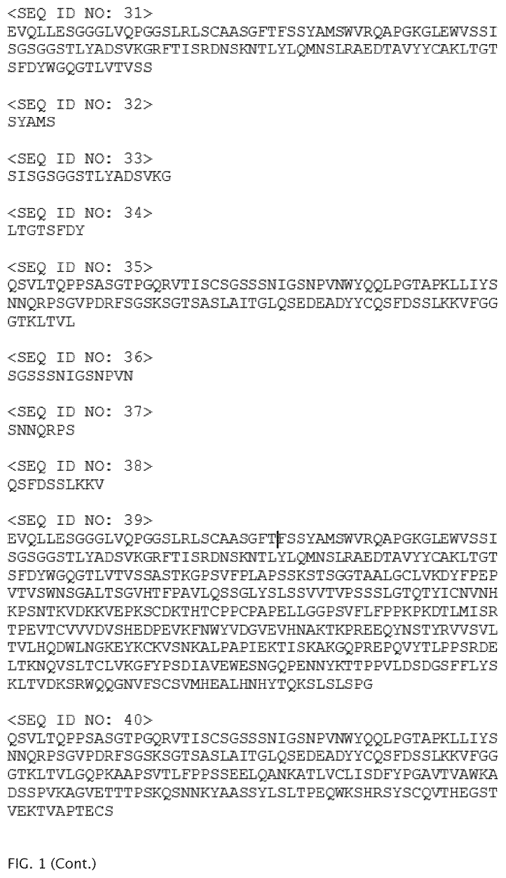

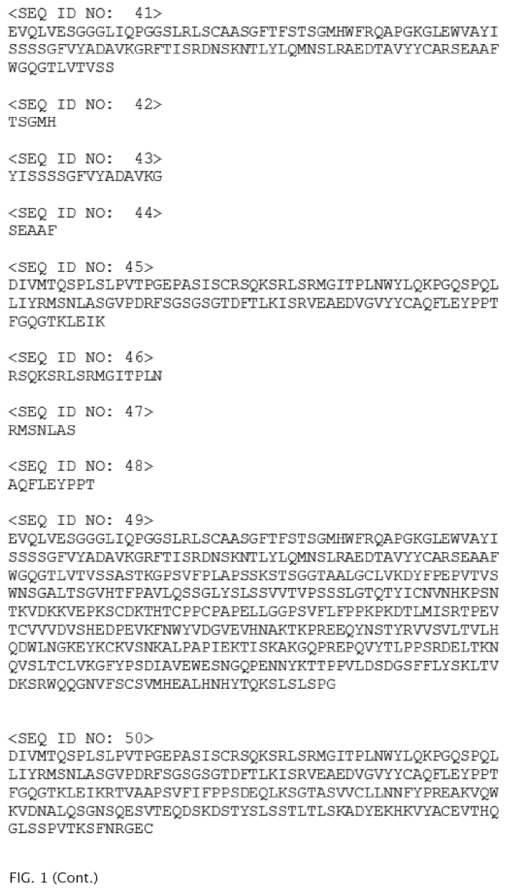

### FIELD OF APPLICATION OF THE INVENTION

The present invention relates to novel conjugates of a binder or a derivative thereof with one or more molecules of an active component, wherein the active component is a NAMPT inhibitor, which is conjugated to the binder via a linker Z′ as described and defined herein, and methods for their preparation, their use for the treatment and/or prophylaxis of disorders, in particular of hyper-proliferative disorders.

### BACKGROUND OF THE INVENTION

Nicotinamide adenine dinucleotide (NAD) is a biologically important coenzyme that plays a critical role in many cell metabolism-related transformations and in cell signaling [Lin, S-J.; Guarente L. Current Opinion Cell Biol. 2003, 15, 241-146; Ziegler M. Eur. J. Biochem. 2000, 267, 1550-1564].

In mammalian cells, the two step salvaging of NAD⁺ from nicotinamide (NAM)—nicotinamide pathway—is the most efficient process compared to the de novo synthesis of NAD⁺ from the essential amino acid L-tryptophan which takes mainly place in the liver [Schramm V. L. et al. PNAS 2009, 106, 13748-13753]. NAMPT (nicotinamide phosphoribosyltransferase also known as pre-B-cell-colony-enhancing factor (PBEF) and visfatin, NMPRT, NMPETase or NAmPRTase, international nomenclature E.C.2.4.2.12) catalyzes the first step of this process, the phosphoribosylation of NAM to NMN (nicotinamide mononucleotide) which is further converted to NAD⁺ by NMNAT (nicotinamide mononucleotide adenylyltransferase). NAMPT is the rate-limiting enzyme in the production of NAD⁺ and its inhibition leads to a rapid depletion of NAD⁺[Deng Y. et al. Bioanalysis 2014, 6, 1145-1457].

In general, an altered cell metabolism is one of the basic characteristics of cancer cells as hypothesized by Otto Heinrich Warburg [Warburg, O. Über den Stoffwechsel der Carcinomzelle. Klin. Wochenschr. 4, 534-536 (1925)]. As cancer cells proliferate continuously, these cells have to adapt to a stressful and dynamic microenvironment. This results in an increased need for energy, macromolecules and the maintenance of the cellular redox status by cancer cells [Cairns R. A. et al. Nature Rev. 2011, 11, 85-95].

With this regard, NAD⁺ is used as electron carrier in glycolysis, which is up-regulated in cancer cells due to the Warburg effect, as well as in mitochondrial oxidative phosphorylation. Further, NAD⁺ serves as a substrate for several enzymes, for example poly-ADP-ribose polymerases (PARPs) and sirtuins (SIRTs) which are involved in DNA repair and gene expression, processes often aberrantly regulated in cancer cells and leading to consumption of NAD⁺ [Berger F et al. 2004 Trends Biochem. Sci. 29, 111-118]. Phosphorylated forms of NAD⁺/NADH also exist and are often employed for biosynthetic and/or cell protection purposes in addition to energy generation. They are also involved in the cellular response to oxidative stress [Massudi H. Redox Rep. 2012, 17, 28-46].

For these reasons, many cancer cells have an increased need for NAD⁺ and its synthesis is constantly required, rendering cancer cells particularly sensitive to NAMPT inhibition.

Moreover, it was demonstrated that NAMPT is implicated in the regulation of cell viability during genotoxic or oxidative stress and that NAMPT inhibitors are potentially useful for the treatment of e.g. inflammation, metabolic disorders and cancer [Tong L. et al. Expert Opin. Ther. Targets 2007, 11, 695-705; Galli, M. et al. Cancer Res. 2010, 70, 8-11, J. Med. Chem 2013, 56, 6279-6296].

Daporinad also known as AP866, FK866, WK175 or WK22 ((E)-N-[4-(I-benzoylpiperidin-4-yl)butyl]-3-(pyridine-3-yl)-acrylamide) is a highly potent and selective inhibitor of NAMPT which interferes with NAD biosynthesis, ATP generation and induces cell death. In vivo efficacy of daporinad was shown in murine renal cell carcinoma model RENCA [Drevs J. et al. Anticancer Res 2003, 23, 4853-4858]. Clinical trials with daporinad have been completed for the treatment of chronic lymphocytic leukemia (CLL), cutaneous T cell lymphoma (CTL), and advanced melanoma [ClinicalTrials.gov Identifier: NCT00435084, NCT00431912, NCT00432107].

CHS-828 also known as GMX1778 (N-[6-(4-chlorophenoxy)hexyl]-N′-cyano-N″-4-pyridinyl-guanidine), an inhibitor of NAMPT as well as an inhibitor of NF-κB pathway activity [Hassan S. B. et al. Anticancer Res 2006, 26, 4431-4436], showed highly cytotoxic effects in vitro and in vivo in human breast and lung cancer cell line-derived in vivo models [Hijarnaa P J et al. Cancer Res. 1999, 59, 5751-5757]. A Phase I study for this compound in patients with solid tumors was published in the year 2002 [Hovstadius P et al. Clin Cancer Res 2002, 9, 2843-2850]. Best observed responses in the clinical trials were stable disease. Therefore, it has been assumed that the lack of significant activity in clinical trials may result from the inability to dose NAMPT inhibitors to higher drug exposures due to dose-limiting toxicities [Sampath D. et al. Pharmacology and Therapeutics 2015, 151, 16-31]. Combining targeting of a cytotoxic drug to cancer cells eg by employing a binder or an antibody may improve the therapeutic window and may thus result in better tolerability and better clinical responses.

The present invention relates to novel conjugates of a binder or a derivative thereof with one or more molecules of an active component, wherein the active component is a NAMPT inhibitor, which is conjugated to the binder via a linker.

A number of chemical compounds have been shown to act as NAMPT inhibitors. For example, Bioorganic & Medicinal Chemistry Letters (2013), 23, 4875-4885; WO 2014111871 and WO 2013067710 discloses 1,3-dihydro-2H-isoindoles as NAMPT inhibitors.

DE10010423, WO9206087 and WO2006064189 disclose 1-alkyl-6-oxo-1,4,5,6-tetrahydropyridazin-3-yl derivatives which may be useful for the treatment of anemia, cardiovascular and diglyceride acyltransferase (DGAT) mediated disorders (e.g. diabetes), respectively.

WO2012067965 discloses 4-oxo-3,4-dihydrophthalazine phenyl cyclic urea derivatives which may be useful as NAMPT and ROCK inhibitors.

Despite the progress made during the last decades in the treatment of uncontrolled proliferative cellular processes in humans and animals, like cancer diseases, there is still a huge unmet medical need to expand therapeutic options especially based on new drugs selectively addressing new targets.

### SUMMARY OF THE INVENTION

Therefore, inhibitors of NAMPT represent valuable compounds that should complement therapeutic options either as single agents or in combination with other drugs, particularly those NAMPT inhibitors with increased selectivity over other biological targets. It is thus an object of the present invention to provide substances which may be of benefit for cancer therapy.

To achieve this object, the invention provides conjugates of a binder or derivatives thereof with one or more active compound molecules, the active compound molecule being a NAMPT inhibitor attached to the binder via a linker Z′. The binder is preferably a binder protein or peptide, particularly preferably a human, humanized or chimeric monoclonal antibody or an antigen-binding fragment thereof.

The conjugate according to the invention can be represented by the general formula:

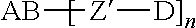

wherein AB stands for a binder, Z′ stands for a linker, n stands for a number between 1 and 50, and D stands for an active component of Formula (I-D):

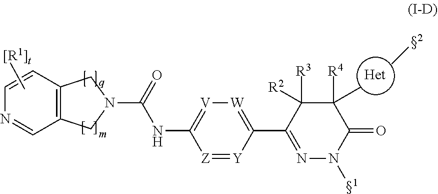

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a carbon or nitrogen atom of ring Het and §¹ represents R^{5b};
Het represents a heteroaryl group optionally substituted with one or more groups independently selected from R⁵;
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂;
t is 0, 1 or 2;
R² represents H, C₁-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷;
R³ represents H, C₁-C₃-alkyl or C₁-C₃-haloalkyl; and
R⁴ represents H, C₁-C₆-alkyl, C₃-C₆-cycloalkyl, C₁-C₄-haloalkyl or phenyl;
or,
R² and R³ together with the carbon to which they are attached form a C₃-C₆-cycloalkyl group or a 5- to 7-membered heterocycloalkyl group containing one heteroatom containing group selected from O, NR, S, S(═O), S(═O)₂, S(═NR)(═NR⁹) and S(═O)(═NR⁸); and
R⁴ represents H, C₁-C₆-alkyl, C₃-C₆-cycloalkyl, C₁-C₄-haloalkyl or phenyl;
or,
R² represents H, C₁-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂; 4- to 7-membered heterocycloalkyl, —SR⁸, —S(═O)R⁸, —S(═O)₂R⁸ and —S(═O)(═NR⁸)R⁹;
R^{5a} represents R⁵, hydrogen or is absent;
R^{5b} represents hydrogen or a group selected from:
   methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
   in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —NH₂, —N(H)R⁶, —N(R⁶)R⁷, —C(═O)OR⁸, —SR⁸, —S(═O)R⁸, —S(═O)₂R⁸ and —S(═O)(═NR⁸)R⁹;
   wherein azetidin-3-yl and oxetan-3-yl are optionally substituted with one or two substituents independently selected from the group consisting of:
   C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, and C₃-C₆-cycloalkyloxy;
   wherein C₃-C₆-cycloalkyl and 5- to 7-membered heterocycloalkyl are optionally substituted with one or more substituents independently selected from the group consisting of:
   hydroxy, halogen, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, C₃-C₆-cycloalkyloxy, —N(R⁵)R⁶, —C(═O)OH, oxo (═O), and —N(H)C(═O)—(C₁-C₃-alkyl);
   wherein phenyl and 5- to 6-membered heteroaryl are optionally substituted with one or more substituents independently selected from the group consisting of:
   halogen, C₁-C₃-alkyl, C₁-C₃-alkoxy-, C₁-C₃-haloalkoxy-, —N(H)R⁶, —N(R⁶)R⁷, —C(═O)OH and —C(═O)O(C₁-C₆-alkyl);
q is 0, 1, 2 or 3,
m is 0, 1, 2 or 3,
with the proviso that q+m is 2, 3 or 4;

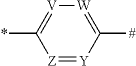

represents a group which is selected from:

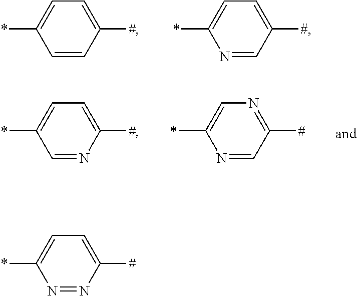

in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷;
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷;
R⁸, R⁹ represent, independently of each other, hydrogen, C₁-C₃-alkyl, C₃-C₆-cycloalkyl, phenyl or C₁-C₃-haloalkyl,
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷;
      or an N-oxide, a salt, a tautomer or a stereoisomer of said compound, or a salt of said N-oxide, tautomer or stereoisomer.

The inventors have found a number of methods to attach the binder to the NAMPT inhibitor in order to achieve the object mentioned above.

According to the invention, the NAMPT inhibitor may be attached to the binder via a linker Z′ at position §¹ or §² in formula (I).

The conjugates according to the invention can have chemically labile linkers, enzymatically labile linkers or stable linkers.

The linker —Z′— may represent one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²-L1-L2-§§
   wherein
   §¹, §² represent the attachment point to D;
   §§ represents the attachment point to AB;
   SG represents an in vivo cleavable group, L1 and L1′ represent, independently of each other, an in vivo non-cleavable organic group, and L2 represents an attachment group.
   SG may represent a 2-8 oligopeptide group, preferably a dipeptide group or a tripeptide group, or a disulfide, a hydrazone, a glycoside, an acetal or an aminal.
   L1, L1′ may represent, independently of each other, a straight-chain or branched hydrocarbon chain having 1 to 40 carbon atoms which may be interrupted once or more than once by one or more groups independently selected from:
   —O—, —S—, —SO—, SO₂, —NH—, —CO—, —NMe-, —NHNH—, —SO₂NHNH—, —NHCO—, —CONH—, —CONHNH—, arylene groups, heteroarylene groups, straight C₁-C₆-alkylene groups, branched C₁-C₆-alkylene groups, C₃-C₇-cyclic alkylene groups and 5- to 10-membered heterocyclic groups having up to 4 heteroatoms selected from the group consisting of N, O and S, —SO— or —SO₂—;
   optionally substituted with one or more substituents selected from the group consisting of halogen, —NHCONH₂, —COOH, —OH, —NH₂, NH—CNNH₂, sulphonamide, sulphone, sulphoxide or sulphonic acid.
   L2 may represent:

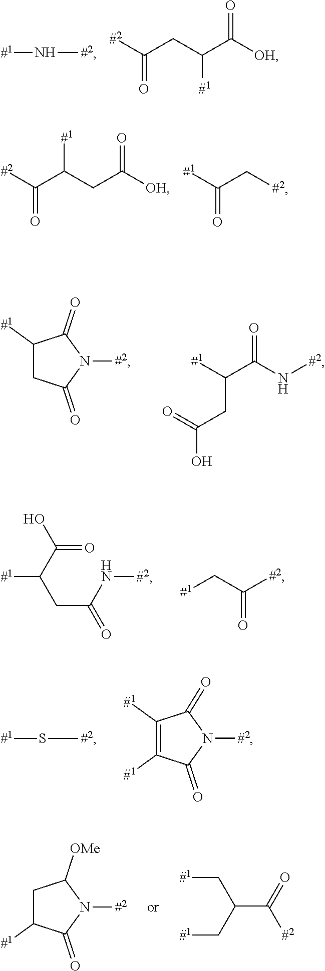

wherein
#¹ represents the attachment point to the binder,
#² represents the attachment point to the group L1, L1′ or SG.

The invention furthermore provides processes for preparing the conjugates according to the invention, and also precursors and intermediates for the preparation.

The preparation of the conjugates according to the invention regularly comprises the following steps:

(i) Preparation of a linker precursor which optionally carries protective groups;
(ii) Conjugation of the linker precursor to the derivative, which optionally carries protective groups, of a low-molecular weight NAMPT inhibitor (preferably a NAMPT inhibitor having Formula (I), giving a NAMPT inhibitor/linker conjugate which optionally carries protective groups;
(iii) Attachment of a reactive group to the NAMPT inhibitor/linker conjugate;
(iv) Removal of any protective groups present in the NAMPT inhibitor/linker conjugate and
(v) Conjugation of the binder to the NAMPT inhibitor/linker conjugate, giving the binder/NAMPT inhibitor conjugate according to the invention.

Attachment of the reactive group may also take place during preparation of the linker precursor (e.g. during step (i) above)) rather than after the construction of an optionally protected NAMPT inhibitor/linker precursor conjugate.

Depending on the linker, succinimide-linked ADCs may, after conjugation, be converted according to Scheme A1 or Scheme A2 into the open-chain succinamides, which may have an advantageous stability profile.

As illustrated above, conjugation of the linker precursor to a low-molecular weight NAMPT inhibitor may take place at position §¹ or §² in formula (I). In the synthesis steps prior to the conjugation, any functional groups present may also be present in protected form. Prior to the conjugation step, these protective groups are removed by known methods of peptide chemistry. Conjugation can take place chemically by various routes. In particular, it is optionally possible to modify the low-molecular weight NAMPT inhibitor for conjugation to the linker, for example by introduction of protective groups or leaving groups to facilitate substitution.

In linker Z′, position #¹ of group L2 preferably reacts with an amino or thiol group on binder AB to form a covalent bond, preferably with a cysteine or a lysine residue in a protein of AB.

The cysteine residue in a protein may of course be present naturally in the protein, may be introduced by biochemical methods or, preferably, may be generated by prior reduction of disulphides of the binder.

### DETAILED DESCRIPTION OF THE INVENTION

### Definitions

Constituents that are optionally substituted as stated herein, may be substituted, unless otherwise noted, one or more times, independently from one another at any possible position. When any variable occurs more than one time in any constituent or in different constituents, each definition is independent. For example, for any component of formula (I) in which R¹, R⁵, R⁶, R⁷, R⁸ and/or R⁹ occur more than one time, each definition of R¹, R⁶, R⁷, R⁸ and R⁹ is independent.

Should a constituent be composed of more than one part the position of a possible substituent can be at any of these parts at any suitable position. A hyphen at the beginning or at the end of the constituent marks the point of attachment to the rest of the molecule.

Should a ring be substituted, the substitutent(s) could be at any suitable position of the ring, also on a ring nitrogen atom if suitable.

The term “comprising” when used in the specification includes “consisting of”.

If it is referred to “as mentioned above”, “mentioned above” or “supra” within the description it is referred to any of the disclosures made within the specification in any of the preceding pages.

“suitable” within the sense of the invention means chemically possible to be made by methods within the knowledge of a skilled person.

The term “compound” as used in, for example, “compound of the present invention”, “compound of the invention”, “compound described supra”, “compound described herein”, “compound defined supra”, “compound defined herein”, used throughout the specification refer to NAMPT inhibitors (e.g. NAMPT inhibitors (D) of formula (I)) and intermediates (e.g. the NAMPT inhibitor-linker-intermediates of general formula (III)) used to prepare the ADC conjugates of the present invention, as well as metabolites of the ADC conjugates of the present invention.

The terms as mentioned in the present text have preferably the following meanings:

The term “halogen atom”, “halo-” or “Hal-” is to be understood as meaning a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “C₁-C₄-haloalkyl” is to be understood as meaning a linear or branched, saturated, monovalent hydrocarbon group in which the term “C₁-C₄-alkyl” is defined supra, and in which one or more hydrogen atoms is replaced by a halogen atom, identically or differently, i.e. one halogen atom being independent from another. Particularly, said halogen atom is F. Said C₁-C₄-haloalkyl group is, for example, —CF₃, —CHF₂, —CH₂F, —CF₂CF₃, —CH₂CH₂F, —CH₂CHF₂, —CH₂CF₃, —CH₂CH₂CF₃, or —CH(CH₂F)₂. Particularly, said group has 1, 2 or 3 carbon atoms.

The term “C₁-C₄-alkoxy” is to be understood as meaning a linear or branched, saturated, monovalent, hydrocarbon group of formula —O—(C₁-C₄-alkyl), in which the term “C₁-C₄-alkyl” is defined supra, e.g. a methyl, ethyl, n-propyl-, iso-propyl, n-butyl- or tert-butyl group, or an isomer thereof. Particularly, said group has 1, 2 or 3 carbon atoms.

The term “C₁-C₄-haloalkoxy” is to be understood as meaning a linear or branched, saturated, monovalent C₁-C₄-alkoxy group, as defined supra, in which one or more of the hydrogen atoms is replaced, identically or differently, by a halogen atom. Particularly, said halogen atom is F. Said C₁-C₄-haloalkoxy group is, for example, —OCF₃, —OCHF₂, —OCH₂F, —OCF₂CF₃, or —OCH₂CF₃. Particularly, said group has 1, 2 or 3 carbon atoms.

The term “C₃-C₆-cycloalkyl” is to be understood as meaning a saturated, monovalent, monocyclic hydrocarbon ring which contains 3, 4, 5 or 6 carbon atoms (“C₃-C₆-cycloalkyl”). Said C₃-C₆-cycloalkyl group is for example, a monocyclic hydrocarbon ring, e.g. a cyclopropyl, cyclobutyl, cyclopentyl or cyclohexyl ring. Particularly, said group has 3 carbon atoms (“C₃-cycloalkyl”), i.e. a cyclopropyl group.

The term “5- to 7-membered heterocycloalkyl” is to be understood as meaning a saturated or partially unsaturated, monovalent, mono- or bicyclic hydrocarbon ring which contains 3, 4, 5 or 6 carbon atoms, and one or two heteroatom-containing group selected from O, N, NR⁸, S, S(═O), S(═O)₂, S(═NR⁸)(═NR⁹) and S(═O)(═NR⁸), preferably O, N, NR⁸, S, S(═O), S(═O)₂, more preferably O, N, NR⁸, in which R⁸ and R⁹ are as defined herein, said heterocycloalkyl group being attached to the rest of the molecule via a carbon atom or, if present, a nitrogen atom, of the heterocycloalkyl ring.

Particularly, without being limited thereto, said heterocycloalkyl can be a 5-membered ring, such as, but not limited to, tetrahydrofuranyl, pyrrolidinyl or pyrrolinyl, or a 6-membered ring, such as, but not limited to, tetrahydropyranyl, piperidinyl, morpholinyl or piperazinyl, or a 7-membered ring, such as, but not limited to, an azepanyl ring, for example. Optionally, said heterocycloalkyl can be benzo fused.

As mentioned supra, said 5- to 7-membered heterocycloalkyl can be partially unsaturated, i.e. it can contain one or more double bonds, such as, without being limited thereto, a 2,5-dihydro-1H-pyrrolyl, for example, or, it may be benzofused, such as, without being limited thereto, a dihydroisoquinolinyl ring, for example.

The term “heteroatom containing group” is understood as meaning a heteroatom, such as, O and S, or a group containing a heteroatom, such as NR^{B}, S(═O), S(═O)₂, S(═NR⁸)(═NR⁹) and S(═O)(═NR⁸).

The term “C₁-C₆”, as used throughout this text, e.g. in the context of the definition of “C₁-C₆-alkyl” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S ¹⁸F, ³⁶Cl, ⁸²Br, ¹²³I, ¹²⁴I, ¹²⁵I, ¹²⁹I and ¹³¹I, respectively. Certain isotopic variations of a compound of the invention, for example, those in which one or more radioactive isotopes such as 3H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence is preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word conjugates, compounds, salts, polymorphs, hydrates, solvates and the like, is used herein, this is taken to mean also a single conjugate, compound, salt, polymorph, isomer, hydrate, solvate or the like.

By “stable compound” is meant a compound that is sufficiently robust to survive isolation to a useful degree of purity from a reaction mixture, and formulation into an efficacious therapeutic agent.

The compounds of this invention optionally contain one or more asymmetric centres, depending upon the location and nature of the various substituents desired. Asymmetric carbon atoms are present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention optionally contain sulphur atoms which are asymmetric, such as an asymmetric sulfoxide, of structure:

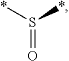

for example,
in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by the techniques provided herein or by (other) standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, the compounds of the present invention may exist as tautomers.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

Accordingly, the present invention includes all possible salts, polymorphs, metabolites, hydrates, solvates, prodrugs (e.g.: esters) thereof, and diastereoisomeric forms of the NAMPT inhibitors or precursors thereof as single salt, polymorph, metabolite, hydrate, solvate, prodrug (e.g.: esters) thereof, or diastereoisomeric form, or as mixture of more than one salt, polymorph, metabolite, hydrate, solvate, prodrug (e.g.: esters) thereof, or diastereoisomeric form in any ratio.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃OOOH”, “x Na⁺”, for example, are to be understood as not a stoichiometric specification, but solely as a salt form.

The salts include water-insoluble and, particularly, water-soluble salts.

This applies analogously to cases in which synthesis intermediates or example compounds or salts thereof have been obtained, by the preparation and/or purification processes described, as solvates, such as hydrates with (if defined) unknown stoichiometric composition.

Furthermore, derivatives of the conjugates described herein and the salts thereof which are converted into a conjugate as described herein or a salt thereof in a biological system (bioprecursors or pro-drugs) are covered by the invention. Said biological system is e.g. a mammalian organism, particularly a human subject. The bioprecursor is, for example, converted into a conjugate as described herein or a salt thereof by metabolic processes.

Furthermore, the present invention includes all possible crystalline forms, or polymorphs, of the conjugates of the present invention, either as single polymorph, or as a mixture of more than one polymorph, in any ratio.

In the context of the properties of the conjugates of the present invention the term “pharmacokinetic profile” means one single parameter or a combination thereof including permeability, bioavailability, exposure, and pharmacodynamic parameters such as duration, or magnitude of pharmacological effect, as measured in a suitable experiment. Conjugates with improved pharmacokinetic profiles can, for example, be used in lower doses to achieve the same effect, may achieve a longer duration of action, or a may achieve a combination of both effects.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered. Any such combination of a compound of formula (I) of the present invention with an anti-cancer agent as defined below is an embodiment of the invention.

The term “(chemotherapeutic) anti-cancer agents”, includes but is not limited to: 131I-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, rucaparib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The invention provides conjugates of a binder or derivative thereof with one or more active compound molecules, the active compound molecule being a NAMPT inhibitor attached to the binder via a linker Z′.

In accordance with a first aspect, the invention relates to a conjugate of a binder or a derivative thereof with one or more molecules of an active compound that has the formula:

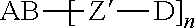

wherein AB stands for a binder, Z′ stands for a linker, n stands for a number between 1 and 50, preferably 1.2 to 20 and especially preferred 2 to 8, and
D stands for an active component of Formula (I-D):

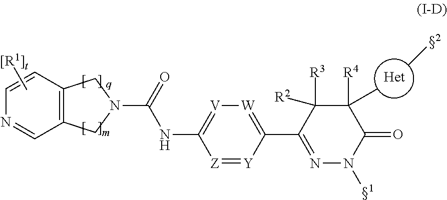

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a carbon or nitrogen atom of ring Het and §¹ represents R^{5b};
Het represents a heteroaryl group optionally substituted with one or more groups independently selected from R⁵;
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂;
t is 0, 1 or 2;
R² represents H, C₁-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷;
   R³ represents H, C₁-C₃-alkyl or C₁-C₃-haloalkyl; and
   R⁴ represents H, C₁-C₆-alkyl, C₃-C₆-cycloalkyl, C₁-C₄-haloalkyl or phenyl;
or,
R² and R³ together with the carbon to which they are attached form a C₃-C₆-cycloalkyl group or a 5- to 7-membered heterocycloalkyl group containing one heteroatom containing group selected from O, NR⁸, S, S(═O), S(═O)₂, S(═NR)(═NR⁹) and S(═O)(═NR⁸); and
R⁴ represents H, C₁-C₆-alkyl, C₃-C₆-cycloalkyl, C₁-C₄-haloalkyl or phenyl;
or,
R² represents H, C₁-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂; 4- to 7-membered heterocycloalkyl, —SR⁸, —S(═O)R⁸, —S(═O)₂R⁸ and —S(═O)(═NR)R⁹;
R^{5a} represents R⁵, hydrogen or is absent;
R^{5b} represents hydrogen or a group selected from:
   methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
   in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —NH₂, —N(H)R⁶, —N(R⁶)R⁷, —C(═O)OR⁸, —SR⁸, —S(═O)R⁸, —S(═O)₂R⁸ and —S(═O)(═NR⁸)R⁹;
   wherein azetidin-3-yl and oxetan-3-yl are optionally substituted with one or two substituents independently selected from the group consisting of:
   C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, and C₃-C₆-cycloalkyloxy;
   wherein C₃-C₆-cycloalkyl and 5- to 7-membered heterocycloalkyl are optionally substituted with one or more substituents independently selected from the group consisting of:
   hydroxy, halogen, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, C₃-C₆-cycloalkyloxy, —N(R⁵)R⁶, —C(═O)OH, oxo (═O), and —N(H)C(═O)—(C₁-C₃-alkyl);
   wherein phenyl and 5- to 6-membered heteroaryl are optionally substituted with one or more substituents independently selected from the group consisting of:
   halogen, C₁-C₃-alkyl, C₁-C₃-alkoxy-, C₁-C₃-haloalkoxy-, —N(H)R⁶, —N(R⁶)R⁷, —C(═O)OH and —C(═O)O(C₁-C₆-alkyl);
q is 0, 1, 2 or 3,
m is 0, 1, 2 or 3,
with the proviso that q+m is 2, 3 or 4;

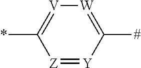

represents a group which is selected from:

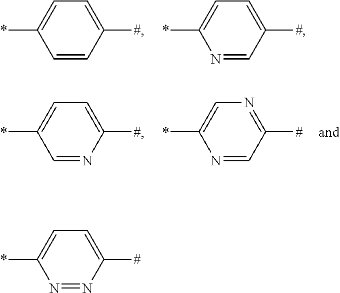

in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷;
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷;
R⁸, R⁹ represent, independently of each other, hydrogen, C₁-C₃-alkyl, C₃-C₆-cycloalkyl, phenyl or C₁-C₃-haloalkyl,
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷; or an N-oxide, a salt, a tautomer or a stereoisomer of said compound, or a salt of said N-oxide, tautomer or stereoisomer.

The binder is preferably a binder peptide or protein such as, for example, an antibody. Furthermore, if n is greater than 1, the linker is preferably attached to different amino acids of the same chemical nature of the binder peptide or protein or derivative thereof. Particular preference is given to binding to different cysteine or lysine residues of the binder, even more preferable is binding to different cysteine residues of the binder.

Binders which can be used according to the invention, NAMPT inhibitors which can be used according to the invention and linkers which can be used according to the invention which can be used in combination without any limitation are described below. In particular, the binders represented in each case as preferred or particularly preferred can be employed in combination with the NAMPT inhibitors represented in each case as preferred or particularly preferred, optionally in combination with the linkers represented in each case as preferred or particularly preferred.

### NAMPT Inhibitors

Despite the fact that various inhibitors of NAMPT are known, there remains a need for selective NAMPT inhibitors to be used for the treatment of diseases such as hyper-proliferative diseases, which offer one or more advantages such as:
improved activity and/or efficacy, allowing e.g. a dose reduction
improved side effect profile, such as fewer undesired side effects, lower intensity of side effects, or reduced (cyto)toxicity
improved physicochemical properties, such as solubility in water, body fluids, and aqueous formulations, e.g. for intravenous administration
improved pharmacokinetic properties, allowing e.g. for dose reduction or an easier dosing scheme
improved duration of action, e.g. by improved pharmacokinetics and/or improved target residence time
easier drug substance manufacturing e.g. by shorter synthetic routes or easier purification.

The NAMPT inhibitors used in the binder drug conjugates according to the invention preferably show anti-proliferative activity in tumor cell lines, such as THP-1, U251 MG, MDA-MB-453 and REC-1, for example.

According to the present invention, the NAMPT inhibitors (D) are described by Formula (I-D):

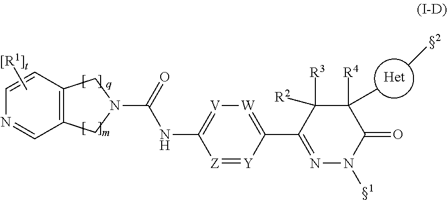

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a carbon or nitrogen atom of ring Het and §¹ represents R^{5b};
Het represents a heteroaryl group optionally substituted with one or more groups independently selected from R⁵;
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂;
t is 0, 1 or 2;
R² represents H, C₁-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷;
R³ represents H, C₁-C₃-alkyl or C₁-C₃-haloalkyl; and
R⁴ represents H, C₁-C₆-alkyl, C₃-C₆-cycloalkyl, C₁-C₄-haloalkyl or phenyl;
or,
R² and R³ together with the carbon to which they are attached form a C₃-C₆-cycloalkyl group or a 5- to 7-membered heterocycloalkyl group containing one heteroatom containing group selected from O, NR⁸, S, S(═O), S(═O)₂, S(═NR)(═NR⁹) and S(═O)(═NR⁸); and
R⁴ represents H, C₁-C₆-alkyl, C₃-C₆-cycloalkyl, C₁-C₄-haloalkyl or phenyl;
or,
R² represents H, C₁-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂; 4- to 7-membered heterocycloalkyl, —SR⁸, —S(═O)R⁸, —S(═O)₂R⁸ and —S(═O)(═NR)R⁹;
R^{5a} represents R⁵, hydrogen or is absent;
R^{5b} represents hydrogen or a group selected from:
   methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
   in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —NH₂, —N(H)R⁶, —N(R⁶)R⁷, —C(═O)OR⁸, —SR⁸, —S(═O)R⁸, —S(═O)₂R⁸ and —S(═O)(═NR⁸)R⁹;
   wherein azetidin-3-yl and oxetan-3-yl are optionally substituted with one or two substituents independently selected from the group consisting of:
   C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, and C₃-C₆-cycloalkyloxy;
   wherein C₃-C₆-cycloalkyl and 5- to 7-membered heterocycloalkyl are optionally substituted with one or more substituents independently selected from the group consisting of:
   hydroxy, halogen, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, C₃-C₆-cycloalkyloxy, —N(R⁵)R⁶, —C(═O)OH, oxo (═O), and —N(H)C(═O)—(C₁-C₃-alkyl);
   wherein phenyl and 5- to 6-membered heteroaryl are optionally substituted with one or more substituents independently selected from the group consisting of:
   halogen, C₁-C₃-alkyl, C₁-C₃-alkoxy-, C₁-C₃-haloalkoxy-, —N(H)R⁶, —N(R⁶)R⁷, —C(═O)OH and —C(═O)O(C₁-C₆-alkyl);
q is 0, 1, 2 or 3,
m is 0, 1, 2 or 3,
with the proviso that q+m is 2, 3 or 4;

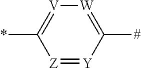

represents a group which is selected from:

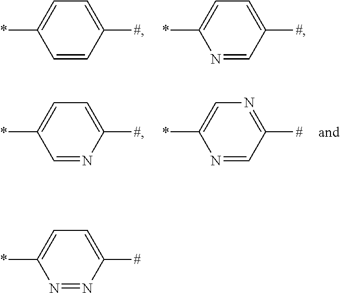

in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷;
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷;
R⁸, R⁹ represent, independently of each other, hydrogen, C₁-C₃-alkyl, C₃-C₆-cycloalkyl, phenyl or C₁-C₃-haloalkyl,
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷;
      or an N-oxide, a salt, a tautomer or a stereoisomer of said compound, or a salt of said N-oxide, tautomer or stereoisomer.

In a second aspect, the invention relates to a conjugate as described supra,

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a carbon or nitrogen atom of ring Het and §¹ represents R^{5b};
Het represents a heteroaryl group, optionally substituted with one or more groups independently selected from R⁵;
t is 0;
R² represents H, C₁-C₆-alkyl, C₃-C₆-cycloalkyl, C₁-C₄-haloalkyl or phenyl,
R³ represents H; and
R⁴ represents H, C₁-C₄-alkyl, or C₁-C₂-haloalkyl;
or,
R² represents H; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂; 4- to 7-membered heterocycloalkyl, —SR⁸, —S(═O)R⁸, —S(═O)₂R⁸ and —S(═O)(═NR)R⁹;
R^{5a} represents R⁵, hydrogen or is absent;
R^{5b} represents hydrogen or a group selected from:
   methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
   in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —C(═O)OH and —N(R⁶)R⁷;
   wherein C₃-C₆-cycloalkyl and 5- to 7-membered heterocycloalkyl are optionally substituted with one or more substituents independently selected from the group consisting of:
   hydroxy, halogen, cyano, C₁-alkyl, C₁-haloalkyl, C₁-alkoxy, C₁-haloalkoxy, and oxo (═O);
   wherein phenyl and 5- to 6-membered heteroaryl are optionally substituted with one or more substituents independently selected from the group consisting of:
   halogen, C₁-C₃-alkyl, C₁-C₃-alkoxy-, C₁-C₃-haloalkoxy-, —C(═O)OH and —C(═O)O(C₁-C₆-alkyl);
q is 1,
m is 1,

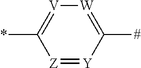

represents a group

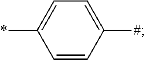

in which * and ^{#} represent the points of attachment of said group with the rest of the compound of formula (I),
R⁶, R⁷ represent, independently of each other, C₁-C₃-alkyl, C₃-C₆-cycloalkyl, phenyl or —C(═O)—(C₁-C₃-alkyl);
R⁸ represents hydrogen, C₁-C₃-alkyl, C₃-C₆-cycloalkyl, phenyl or C₁-C₃-haloalkyl;
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷;
   or an N-oxide, a salt, a tautomer or a stereoisomer of said compound, or a salt of said N-oxide, tautomer or stereoisomer.

In a third aspect, the invention relates to a conjugate as described supra,

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a nitrogen atom of ring Het and §¹ represents R^{5b};
Het represents a heteroaryl group optionally substituted with one or more groups independently selected from R⁵;
t is 0;
R² represents H,
R³ represents H; and
R⁴ represents H, C₁-alkyl, or C₁-haloalkyl;
or,
R² represents H; and
R³ and R⁴ together form a bond;
R⁵ represents, independently of each other, halogen, hydroxy, C₁-alkyl; 5- to 6-membered heterocycloalkyl, —SR⁸, —S(═O)R⁸, and —S(═O)₂R⁸;
R^{5a} represents hydrogen or is absent;
R^{5b} represents hydrogen or a group selected from:
   methyl, C₂-C₃-alkyl,
   wherein C₂-C₃-alkyl is optionally substituted with one or more substituents independently selected from the group consisting of:
   halogen;
q is 1,
m is 1,

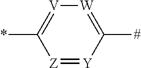

represents a group

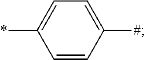

in which * and ^{#} represent the points of attachment of said group with the rest of the compound of formula (I),
R⁸ represents hydrogen, C₁-C₃-alkyl, or phenyl;
   wherein said phenyl is optionally substituted with one or more substituents independently selected from the group consisting of:
   C₁-alkyl;
      or an N-oxide, a salt, a tautomer or a stereoisomer of said compound, or a salt of said N-oxide, tautomer or stereoisomer.

In a fourth aspect, the invention relates to a conjugate as described supra,

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a nitrogen atom of ring Het and §¹ represents R^{5b};
Het represents a heteroaryl group optionally substituted with one or more groups independently selected from R⁵;
t is 0;
R² represents H,
R³ represents H; and
R⁴ represents H, or C₁-haloalkyl;
or,
R² represents H; and
R³ and R⁴ together form a bond;
R⁵ represents, independently of each other, C₁-alkyl; 6-membered heterocycloalkyl, and —S(═O)₂R⁸;
R^{5a} represents hydrogen or is absent;
R^{5b} represents hydrogen or a group selected from:
   C₂-alkyl optionally substituted with one or more fluorine atoms;
q is 1,
m is 1,

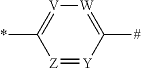

represents a group

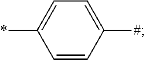

in which * and ^{#} represent the points of attachment of said group with the rest of the compound of formula (I),
R⁸ represents phenyl optionally substituted with one or more C₁-alkyl;
   or an N-oxide, a salt, a tautomer or a stereoisomer of said compound, or a salt of said N-oxide, tautomer or stereoisomer.

According to another aspect, the invention relates to a conjugate as described supra,

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a nitrogen atom of ring Het and §¹ represents R^{5b};
Het represents a heteroaryl group optionally substituted with one or more groups independently selected from R⁵;
t is 0;
R² represents H,
R³ represents H; and
R⁴ represents H;
or,
R² represents H; and
R³ and R⁴ together form a bond;
R⁵ represents C₁-alkyl;
R^{5a} represents hydrogen or is absent;
R^{5b} represents a group —CH₂CF₃;
q is 1,
m is 1,

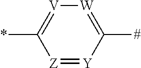

represents a group

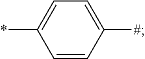

in which * and ^{#} represent the points of attachment of said group with the rest of the compound of formula (I),
or an N-oxide, a salt, a tautomer or a stereoisomer of said compound, or a salt of said N-oxide, tautomer or stereoisomer.

In another aspect, the NAMPT inhibitors (D) are described by Formula (III-D):

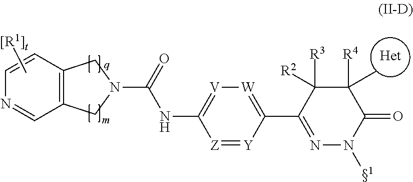

wherein R¹, R², R³, R⁴, §¹, Het, t, q, m, V, W, Z and Y are as defined herein.

In another aspect, the NAMPT inhibitors (D) are described by Formula (III-D):

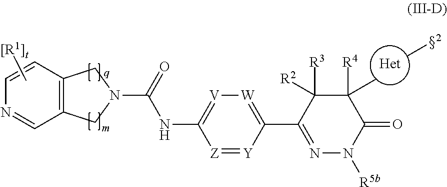

wherein R¹, R², R³, R⁴, §², Het, t, q, m, V, W, Z and Y are as defined herein.

In a further embodiment of the above-mentioned aspects, the invention relates to a conjugate as described supra,

wherein:
§¹ or §² represent the point of attachment to linker Z′, with the proviso that:
when linker Z′ is connected at §¹, then §² represents R^{5a}, and
when linker Z′ is connected at §², then linker Z′ is connected to a carbon or nitrogen atom of ring Het and §¹ represents R^{5b}.

In a further embodiment of the above-mentioned aspects, the invention relates to a conjugate as described supra, wherein:

Het represents a heteroaryl group optionally substituted with one or more groups independently selected from R⁵.

In a further embodiment of the above-mentioned aspects, the invention relates to a conjugate as described supra, wherein:
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂.

In a further embodiment of the above-mentioned aspects, the invention relates to a conjugate as described supra, wherein:
t is 0, 1 or 2.

In a further embodiment of the above-mentioned aspects, the invention relates to a conjugate as described supra, wherein:
t is 0 or 1.

In a further embodiment of the above-mentioned aspects, the invention relates to a conjugate as described supra, wherein:
t is 0.

In a further embodiment of the above-mentioned aspects, the invention relates to a conjugate as described supra, wherein:
R² represents H, 1-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷;
R³ represents H, C₁-C₃-alkyl or C₁-C₃-haloalkyl; and
R⁴ represents H, 1-C₆-alkyl, C₃-C₆-cycloalkyl, C₁-C₄-haloalkyl or phenyl.

In a further embodiment of the above-mentioned aspects, the invention relates to a conjugate as described supra, wherein:
R² represents H, C₁-C₆-alkyl, C₃-C₆-cycloalkyl, C₁-C₄-haloalkyl or phenyl,
R³ represents H; and
R⁴ represents H, C₁-C₄-alkyl, or C₁-C₂-haloalkyl.

In a further embodiment of the above-mentioned aspects, the invention relates to a conjugate as described supra, wherein:
R² represents H,
R³ represents H; and
R⁴ represents H, C₁-alkyl, or C₁-haloalkyl.

In a further embodiment of the above-mentioned aspects, the invention relates to a conjugate as described supra, wherein:
R² represents H,
R³ represents H; and
R⁴ represents H, or C₁-haloalkyl.

In a further embodiment of the above-mentioned aspects, the invention relates to a conjugate as described supra, wherein:
R² represents H,
R³ represents H; and
R⁴ represents H;

In a further embodiment of the above-mentioned aspects, the invention relates to a conjugate as described supra, wherein:
R² and R³ together with the carbon to which they are attached form a C₃-C₆-cycloalkyl group or a 5- to 7-membered heterocycloalkyl group containing one heteroatom containing group selected from O, NR⁸, S, S(═O), S(═O)₂, S(═NR)(═NR⁹) and S(═O)(═NR⁸); and
R⁴ represents H, 1-C₆-alkyl, C₃-C₆-cycloalkyl, C₁-C₄-haloalkyl or phenyl.

In a further embodiment of the above-mentioned aspects, the invention relates to a conjugate as described supra, wherein:
R² represents H, 1-C₆-alkyl, C₃-C₆-cycloalkyl, C₁-C₄-haloalkyl or phenyl,
   wherein phenyl is optionally substituted with one or more substituents independently selected from the group consisting of:
   halogen, C₁-C₃-alkyl, C₁-C₃-alkoxy, C₁-C₃-haloalkoxy, —N(H)R⁶, and —N(R⁶)R⁷; and
R³ and R⁴ together form a bond.

In a further embodiment of the above-mentioned aspects, the invention relates to a conjugate as described supra, wherein:
R² represents H; and
R³ and R⁴ together form a bond.

In a further embodiment of the above-mentioned aspects, the invention relates to a conjugate as described supra, wherein:
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷, —NH₂, 4- to 7-membered heterocycloalkyl, —SR⁸, —S(═O)R⁸, —S(═O)₂R⁸ or —S(═O)(═NR)R⁹.

In a further embodiment of the above-mentioned aspects, the invention relates to a conjugate as described supra, wherein:
R⁵ represents, independently of each other, halogen, hydroxy, C₁-alkyl, C₁-haloalkyl, C₁-alkoxy, C₁-haloalkoxy, —N(H)R⁶, —N(R⁶)R⁷ or —NH₂, 4- to 7-membered heterocycloalkyl, —SR⁸, —S(═O)R⁸, —S(═O)₂R⁸ and —S(═O)(═NR⁸)R⁹.

In a further embodiment of the above-mentioned aspects, the invention relates to a conjugate as described supra, wherein:
R⁵ represents, independently of each other, halogen, hydroxy, C₁-alkyl, 5- to 6-membered heterocycloalkyl, —SR⁸, —S(═O)R⁸ or —S(═O)₂R⁸.

In a further embodiment of the above-mentioned aspects, the invention relates to a conjugate as described supra, wherein:
R⁵ represents, independently of each other, C₁-alkyl, 6-membered heterocycloalkyl, or —S(═O)₂R⁸.

In a further embodiment of the above-mentioned aspects, the invention relates to a conjugate as described supra, wherein:
R⁵ represents C₁-alkyl.

In a further embodiment of the above-mentioned aspects, the invention relates to a conjugate as described supra, wherein:
R^{5a} represents R⁵, hydrogen or is absent.

In a further embodiment of the above-mentioned aspects, the invention relates to a conjugate as described supra, wherein:
R^{5a} represents hydrogen or is absent.

In a further embodiment of the above-mentioned aspects, the invention relates to a conjugate as described supra, wherein:
R^{5b} represents hydrogen or a group selected from:
   methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
   in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —NH₂, —N(H)R⁶, —N(R⁶)R⁷, —C(═O)OR⁸, —SR⁸, —S(═O)R⁸, —S(═O)₂R⁸ and —S(═O)(═NR⁸)R⁹;
   wherein azetidin-3-yl and oxetan-3-yl are optionally substituted with one or two substituents independently selected from the group consisting of:
   C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, and C₃-C₆-cycloalkyloxy;
   wherein C₃-C₆-cycloalkyl and 5- to 7-membered heterocycloalkyl are optionally substituted with one or more substituents independently selected from the group consisting of:
   hydroxy, halogen, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, C₃-C₆-cycloalkyloxy, —N(R⁵)R⁶, —C(═O)OH, oxo (═O), and —N(H)C(═O)—(C₁-C₃-alkyl);
   wherein phenyl and 5- to 6-membered heteroaryl are optionally substituted with one or more substituents independently selected from the group consisting of:
   halogen, C₁-C₃-alkyl, C₁-C₃-alkoxy-, C₁-C₃-haloalkoxy-, —N(H)R⁶, —N(R⁶)R⁷, —C(═O)OH and —C(═O)O(C₁-C₆-alkyl).

In a further embodiment of the above-mentioned aspects, the invention relates to a conjugate as described supra, wherein:
R^{5b} represents hydrogen or a group selected from:
   methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
   in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —C(═O)OH and —N(R⁶)R⁷;
   wherein C₃-C₆-cycloalkyl and 5- to 7-membered heterocycloalkyl are optionally substituted with one or more substituents independently selected from the group consisting of:
   hydroxy, halogen, cyano, C₁-alkyl, C₁-haloalkyl, C₁-alkoxy, C₁-haloalkoxy, and oxo (═O);
   wherein phenyl and 5- to 6-membered heteroaryl are optionally substituted with one or more substituents independently selected from the group consisting of:
   halogen, C₁-C₃-alkyl, C₁-C₃-alkoxy-, C₁-C₃-haloalkoxy-, —C(═O)OH and —C(═O)O(C₁-C₆-alkyl).

In a further embodiment of the above-mentioned aspects, the invention relates to a conjugate as described supra, wherein:
R^{5b} represents hydrogen or a group selected from:
   methyl, C₂-C₃-alkyl,
   wherein C₂-C₃-alkyl is optionally substituted with one or more substituents independently selected from the group consisting of:
   halogen.

In a further embodiment of the above-mentioned aspects, the invention relates to a conjugate as described supra, wherein:
R^{5b} represents hydrogen or a group selected from:
   C₂-alkyl optionally substituted with one or more fluorine atoms.

In a further embodiment of the above-mentioned aspects, the invention relates to a conjugate as described supra, wherein:
R^{5b} represents a group —CH₂CF₃.

In a further embodiment of the above-mentioned aspects, the invention relates to a conjugate as described supra, wherein:
R^{5b} represents hydrogen or a group selected from:
   methyl, C₂-C₆-alkyl, C₃-C₆-cycloalkyl and (C₃-C₆-cycloalkyl)-(C₁-C₆-alkyl)-,
   wherein C₂-C₆-alkyl is optionally substituted with one or more substituents independently selected from the group consisting of:
   halogen, hydroxy, C₁-C₃-alkoxy, C₁-C₃-haloalkoxy, oxo (═O), —C(═O)OH and —N(R⁶)R⁷;
   wherein C₃-C₆-cycloalkyl is optionally substituted with one or more substituents independently selected from the group consisting of:
   hydroxy, halogen, cyano, C₁-alkyl, C₁-haloalkyl, C₁-alkoxy, C₁-haloalkoxy, and oxo (═O).

In a further embodiment of the above-mentioned aspects, the invention relates to a conjugate as described supra, wherein:
R^{5b} represents hydrogen or a group selected from:
   methyl, C₂-C₆-alkyl, C₃-C₆-cycloalkyl and (C₃-C₆-cycloalkyl)-(C₁-C₆-alkyl)-,
   wherein C₂-C₆-alkyl is optionally substituted with one or more halogen atoms,
   wherein C₃-C₆-cycloalkyl is optionally substituted with one or more halogen atoms.

In a further embodiment of the above-mentioned aspects, the invention relates to a conjugate as described supra, wherein:
q is 0, 1, 2 or 3,
m is 0, 1, 2 or 3,
with the proviso that q+m is 2, 3 or 4.

In a further embodiment of the above-mentioned aspects, the invention relates to a conjugate as described supra, wherein:
q is 1,
m is 1.

In a further embodiment of the above-mentioned aspects, the invention relates to a conjugate as described supra, wherein:

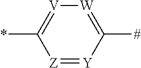

represents a group which is selected from:

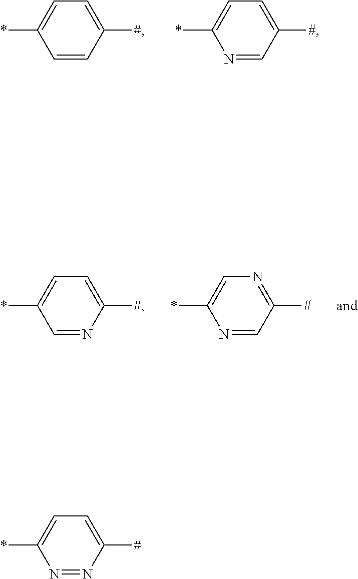

in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷.

In a further embodiment of the above-mentioned aspects, the invention relates to a conjugate as described supra, wherein:

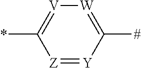

represents a group

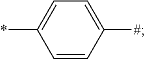

in which * and ^{#} represent the points of attachment of said group with the rest of the compound of formula (I).

In a further embodiment of the above-mentioned aspects, the invention relates to a conjugate as described supra, wherein:
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷.

In a further embodiment of the above-mentioned aspects, the invention relates to a conjugate as described supra, wherein:
R⁶, R⁷ represent, independently of each other, C₁-C₃-alkyl, C₃-C₆-cycloalkyl, phenyl or —C(═O)—(C₁-C₃-alkyl).

In a further embodiment of the above-mentioned aspects, the invention relates to a conjugate as described supra, wherein:
R⁸, R⁹ represent, independently of each other, hydrogen, C₁-C₃-alkyl, C₃-C₆-cycloalkyl, phenyl or C₁-C₃-haloalkyl,
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷.

In a further embodiment of the above-mentioned aspects, the invention relates to a conjugate as described supra, wherein:
R⁸ represents hydrogen, C₁-C₃-alkyl, C₃-C₆-cycloalkyl, phenyl or C₁-C₃-haloalkyl;
   wherein said phenyl is optionally substituted with one or more substituents independently selected from the group consisting of:
   halogen, C₁-C₃-alkyl, C₁-C₃-alkoxy, C₁-C₃-haloalkoxy, —NH₂, —N(H)R⁶, and —N(R⁶)R⁷.

In a further embodiment of the above-mentioned aspects, the invention relates to a conjugate as described supra, wherein:
R⁸ represents hydrogen, C₁-C₃-alkyl, or phenyl;
   wherein said phenyl is optionally substituted with one or more substituents independently selected from the group consisting of:
   C₁-alkyl.

In a further embodiment of the above-mentioned aspects, the invention relates to a conjugate as described supra, wherein:
R⁸ represents phenyl optionally substituted with one or more C₁-alkyl.

A further aspect of the invention are conjugates as described supra, which are present as their salts.

Yet another aspect of the invention are conjugates as described supra in which the salt is a pharmaceutically acceptable salt.

It is to be understood that the present invention relates to any sub-combination within any embodiment or aspect of the present invention of conjugates as described supra.

More particularly still, the present invention covers conjugates that are disclosed in the Example section of this text, infra.

In accordance with another aspect, the present invention covers methods of preparing conjugates of the present invention, said methods comprising the steps as described in the Experimental Section herein.

### Linkers

The literature discloses various options for covalently coupling (conjugating) organic molecules to binders such as, for example antibodies (see, for example, K. Lang and J. W. Chin. Chem. Rev. 2014, 114, 4764-4806, M. Rashidian et al. Bioconjugate Chem. 2013, 24, 1277-1294). Preference according to the invention is given to conjugation of the NAMPT inhibitors to an antibody via one or more sulphur atoms of cysteine residues of the antibody which are either already present as free thiols or generated by reduction of disulphide bridges, and/or via one or more NH groups of lysine residues of the antibody. However, it is also possible to attach the NAMPT inhibitor to the antibody via tyrosine residues, via glutamine residues, via residues of unnatural amino acids, via free carboxyl groups or via sugar residues of the antibody. For coupling, use is made of linkers. Linkers can be categorized into the group of the linkers which can be cleaved in vivo and the group of the linkers which are stable in vivo (see L. Ducry and B. Stump, Bioconjugate Chem. 21, 5-13 (2010)). The linkers which can be cleaved in vivo have a group which can be cleaved in vivo, where, in turn, a distinction may be made between groups which are chemically cleavable in vivo and groups which are enzymatically cleavable in vivo. “Chemically cleavable in vivo” and “enzymatically cleavable in vivo” means that the linkers or groups are cleaved at or in the target cell by the chemically or enzymatically different environment therein (e.g. lower pH; elevated glutathione concentration; presence of lysosomal enzymes such as cathepsin or plasmin, or glyosidases such as, for example, ß-glucuronidases), thus releasing the low-molecular weight NAMPT inhibitor or a derivative thereof. Groups which can be cleaved chemically in vivo are in particular disulphide, hydrazone, acetal and aminal groups; groups which can be cleaved enzymatically in vivo are in particular the 2-8-oligopeptide group, especially a dipeptide group, a tripeptide group or a glycoside group. Peptide cleavage sites are disclosed in Bioconjugate Chem. 2002, 13, 855-869, and Bioorganic & Medicinal Chemistry Letters 8 (1998) 3341-3346 and also Bioconjugate Chem. 1998, 9, 618-626. These include, for example, valine-alanine, valine-lysine, valine-citrulline, alanine-lysine and phenylalanine-lysine (optionally with additional amide group).

Linkers which are stable in vivo are distinguished by a high stability (preferably less than 5% metabolites after 24 hours in plasma) and do not have the chemically or enzymatically in vivo cleavable groups mentioned herein.

In accordance with a fifth aspect, the invention relates to a conjugate as described supra, wherein the linker —Z′— represents one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²_L1-L2-§§
   wherein
   §¹, §² represent the attachment point to D;
   §§ represents the attachment point to AB;
   SG represents an in vivo cleavable group, L1 and L1′ represent, independently of each other, an in vivo non-cleavable organic group, and L2 represents an attachment group.

Attachment group L2 represents a coupling group to the binder or a single bond. Here, coupling is preferably to a cysteine residue or a lysine residue of the binder. Alternatively, coupling can be to a tyrosine residue, glutamine residue or to an unnatural amino acid of the binder. The unnatural amino acids may contain, for example, aldehyde or keto groups (such as, for example, formylglycine) or azide or alkyne groups (see Lan & Chin, Cellular Incorporation of Unnatural Amino Acids and Bioorthogonal Labeling of Proteins, Chem. Rev. 2014, 114, 4764-4806).

In accordance with a sixth aspect, the invention relates to a conjugate as described supra, wherein the in vivo cleavable group SG represents a 2-8 oligopeptide group, preferably a dipeptide group or a tripeptide group, or a disulfide, a hydrazone, a glycoside, an acetal or an aminal.

In accordance with a seventh aspect, the invention relates to a conjugate as described supra, wherein L1 and L1′ represent, independently of each other, a straight-chain or branched hydrocarbon chain having 1 to 40 carbon atoms which may be interrupted once or more than once by one or more groups independently selected from:

—O—, —S—, —SO—, SO₂, —NH—, —CO—, —NMe-, —NHNH—, —SO₂NHNH—, —NHCO—, —CONH—, —CONHNH—, arylene groups, heteroarylene groups, straight C₁-C₆-alkylene groups, branched C₁-C₆-alkylene groups, C₃-C₇-cyclic alkylene groups and 5- to 10-membered heterocyclic groups having up to 4 heteroatoms selected from the group consisting of N, O and S, —SO— or —SO₂—;
optionally substituted with one or more substituents selected from the group consisting of halogen, —NHCONH₂, —COOH, —OH, —NH₂, NH—CNNH₂, sulphonamide, sulphone, sulphoxide or sulphonic acid.

When the linker is attached to a cysteine side chain or a cysteine residue, L2 is preferably derived from a group which reacts with the sulphhydryl group of the cysteine. These include haloacetyls, maleimides, aziridines, acryloyls, arylating compounds, vinylsulphones, pyridyl disulphides, TNB thiols and disulphide-reducing agents. These groups generally react in an electrophilic manner with the sulphhydryl bond, forming a sulphide (e.g. thioether) or disulphide bridge.

In accordance with a eighth aspect, the invention relates to a conjugate as described supra, wherein L2 represents:

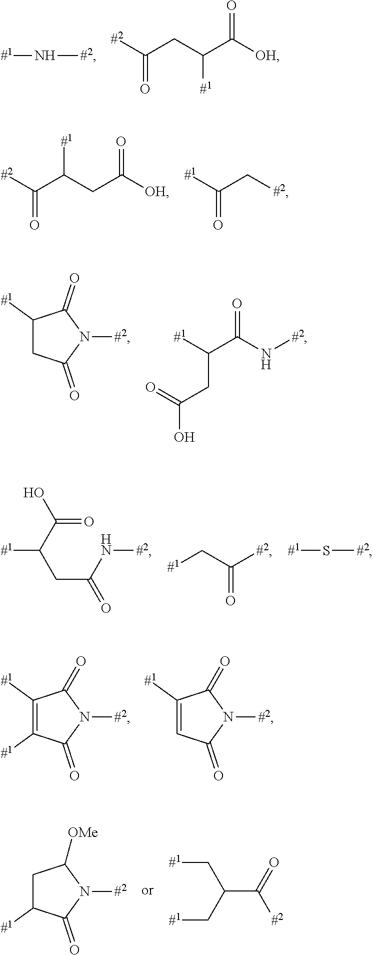

wherein
#¹ represents the attachment point to the binder,
#² represents the attachment point to the group L1, L1′ or SG.

In accordance with a ninth aspect, the invention relates to a conjugate as described supra, wherein the linker —Z′— represents one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²_L1-L2-§§
   wherein
§¹, §² represent the attachment point to D;
§§ represents the attachment point to AB;
SG represents a 2-8 oligopeptide group, preferably a dipeptide group or a tripeptide group, or a disulfide, a hydrazone, a glycoside, an acetal or an aminal;
L1, L1′ represent, independently of each other, a straight-chain or branched hydrocarbon chain having 1 to 40 carbon atoms which may be interrupted once or more than once by one or more groups independently selected from:
   —O—, —S—, —SO—, SO₂, —NH—, —CO—, —NMe-, —NHNH—, —SO₂NHNH—, —NHCO—, —CONH—, —CONHNH—, arylene groups, heteroarylene groups, straight C₁-C₆-alkylene groups, branched C₁-C₆-alkylene groups, C₃-C₇-cyclic alkylene groups and 5- to 10-membered heterocyclic groups having up to 4 heteroatoms selected from the group consisting of N, O and S, —SO— or —SO₂—;
   optionally substituted with one or more substituents selected from the group consisting of halogen, —NHCONH₂, —COOH, —OH, —NH₂, NH—CNNH₂, sulphonamide, sulphone, sulphoxide or sulphonic acid;
L2 represents:

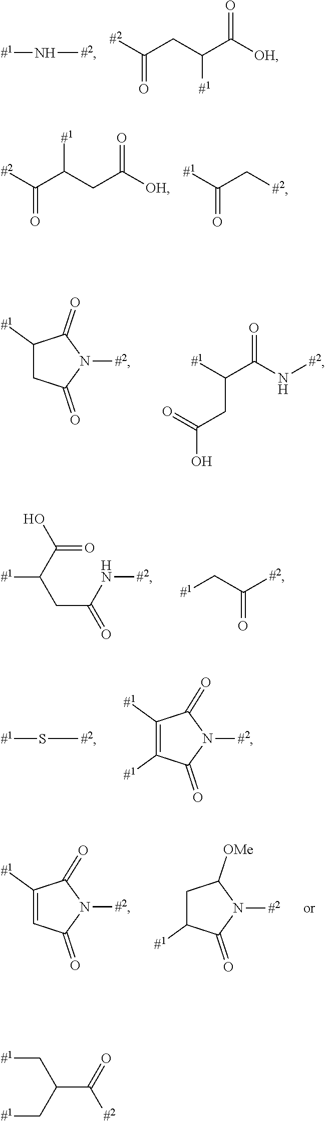

wherein
#¹ represents the attachment point to the binder,
#² represents the attachment point to the group L1, L1′ or SG.

In accordance with an tenth aspect, the invention relates to a conjugate as described supra, wherein L2 represents one or more of the following three formulae:

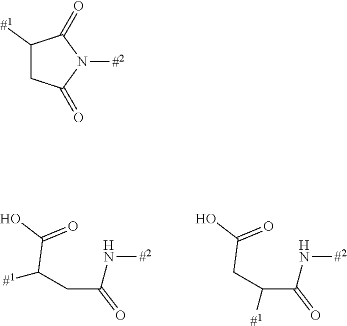

wherein
#¹ represents the attachment point to the binder,
#² represents the attachment point to the group L1, L1′ or SG,
   wherein in a preferred embodiment over 60% of the attachment points to the binder, even more preferred over 80% of the attachment points to the binder, preferably over 90% of the attachment points to the binder, preferably over 95% of the attachment points to the binder in respect to the total number of attachments of the linker to the binder, are represented by one of the two structures:

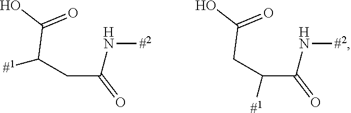

wherein, in a particularly preferred embodiment, the amide group at #² is connected to L1, L1′ or SG via the group —CH₂—C(O)—.

In accordance with a eleventh aspect, the invention relates to a conjugate as described supra, wherein SG is a 2-8 oligopeptide.

In accordance with a twelfth aspect, the invention relates to a conjugate as described supra, wherein the 2-8 oligopeptide consists of amino acids selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, citrulline and valine.

In accordance with a thirteenth aspect, the invention relates to a conjugate as described supra, wherein L1 and L1′ represent, independently of each other, a straight-chain or branched hydrocarbon chain having 1 to 20 carbon atoms which may be interrupted once or more than once by one or more groups independently selected from:
—O—, —NH—, —CO—, —NHCO—, —CONH—;
   in which * and ^{#} represent the points of attachment of said group with the rest of the compound, being optionally substituted with one or more substituents independently selected from the group consisting of —F, —Cl, —COOH, —OH, and —NH₂.

In accordance with a fourteenth aspect, the invention relates to a conjugate as described supra, wherein L1 and L1′ represent, independently of each other, one of the general structures (iv) or (v):
(iv) -A′-(NR¹⁰CO)—B′—
(v) -A′-(CONR¹⁰)—B′—
   wherein:
A′ represents C₁-C₆ alkyl, (C₁-C₂ alkyl)-(phenylene), and (C₁-C₃ alkyl)-(NR¹¹)—(C₂ alkyl); optionally substituted with one or more substituents independently selected from —F and —Cl;
B′ represents a straight-chain or branched hydrocarbon chain having 1 to 20 carbon atoms which may be interrupted once or more than once by one or more groups independently selected from: —O—, —NH—, —CO—, —NHCO—, and —CONH—; optionally substituted with —COOH;
R¹⁰, R¹¹ represent, independently of each other hydrogen or C₁-C₃ alkyl; or
R¹⁰, R¹¹ together with the nitrogens to which they are attached form a 6-membered nitrogen containing heterocycloalkyl group.

In accordance with a fifteenth aspect, the invention relates to a conjugate as described supra,

wherein the linker —Z′— represents, one of the general structures (vi) to (vii):
(vi) §¹-A²-(NR¹⁰-SG′-CO)—B²-L2-§§
(vii) §²-A²-(NR¹⁰-SG′-CO)—B²-L2-§§
§¹, §² represent the attachment point to D;
§§ represents the attachment point to AB;
L2 is as defined in any one of claims 8 to 10;
SG′ is optionally present and, when present, represents:
SG as defined in any one of the preceeding claims, or
   one amino acid (lysine, asparagine) optionally substituted with —[CH₂—CH₂O]ₒCH₃; —C(═O)[CH₂—CH₂O]ₒCH₃; —NHC(═O)[CH₂—CH₂O]ₒCH₃,
o represents an integer from 3 to 9, preferably 4 to 8;
A² represents C₂-C₆-alkyl; optionally substituted with one or more substituents independently selected from —F, —Cl and —COOH;
B² represents a straight-chain or branched hydrocarbon chain having 1 to 20 carbon atoms which may be interrupted once or more than once by one or more groups independently selected from —O—, —NH—, —CO—, —NHCO—, and —CONH—; optionally substituted with —COOH;
R¹⁰ represents hydrogen or C₁-C₃ alkyl.

According to another aspect, the invention relates to a conjugate of general formula (II):

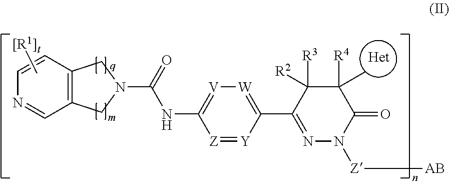

wherein AB stands for a binder, Z′ stands for a linker, n stands for a number between 1 and 50, preferably 1.2 to 20 and especially preferred 2 to 8;
wherein:
wherein R¹, R², R³, R⁴, Het, t, q, m, V, W, Z and Y are as defined herein or as defined in any one of claims 1 to 4;
—Z′— represents one of the following general structures (i) to (iii):
(i) §¹-L1-SG-L2-§§
(ii) §¹-L1-SG-L1′-L2-§§
(iii) §¹-L1-L2-§§
   wherein
§¹ represents the attachment point to the pyridazinone ring;
§§ represents the attachment point to AB;
SG represents a 2-8 oligopeptide group, preferably a dipeptide group or a tripeptide group, or a disulfide, a hydrazone, a glycoside, an acetal or an aminal;
L1, L1′ represent, independently of each other, a straight-chain or branched hydrocarbon chain having 1 to 40 carbon atoms which may be interrupted once or more than once by one or more of —O—, —S—, —SO—, SO₂, —NH—, —CO—, —NMe-, —NHNH—, —SO₂NHNH—, —NHCO—, —CONH—, —CONHNH—, arylene groups, heteroarylene groups, cyclic alkylene groups and 5- to 10-membered heterocyclic groups having up to 4 heteroatoms selected from the group consisting of N, O and S, —SO— or —SO₂—;
   in which * and ^{#} represent the points of attachment of said group with the rest of the compound,
   optionally substituted with one or more substituents selected from the group consisting of halogen, —NHCONH₂, —COOH, —OH, —NH₂, NH—CNNH₂, sulphonamide, sulphone, sulphoxide or sulphonic acid;
L2 represents:

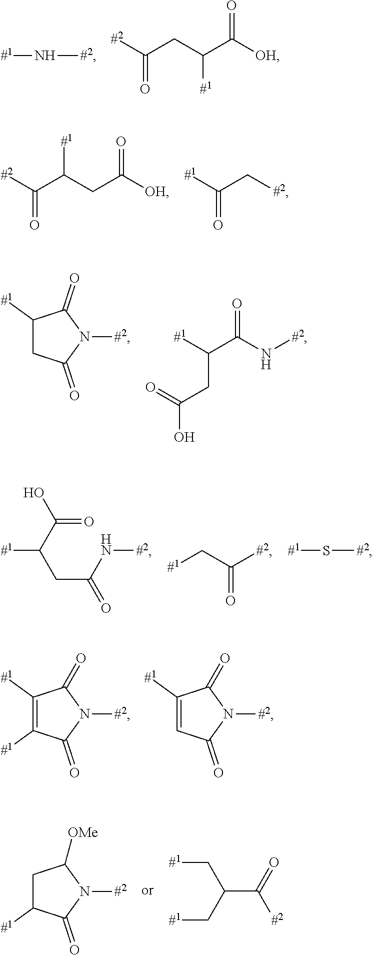

wherein
#¹ represents the attachment point to the binder,
#² represents the attachment point to the group L1, L1′ or SG;
   or the enantiomers, diastereomers, salts, solvates or salts of solvates thereof.

According to another aspect, the invention relates to a conjugate of general formula (III):

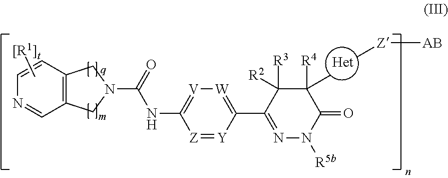

wherein AB stands for a binder, Z′ stands for a linker, n stands for a number between 1 and 50, preferably 1.2 to 20 and especially preferred 2 to 8;
wherein:
wherein R¹, R², R³, R⁴, R^{5b}, Het, t, q, m, V, W, Z and Y are as defined herein or as defined in any one of claims 1 to 4;
—Z′— represents one of the following general structures (i) to (iii):
(i) §²-L1-SG-L2-§§
(ii) §²-L1-SG-L1′-L2-§§
(iii) §²-L1-L2-§§
   wherein
§² represents the attachment point to ring Het;
§§ represents the attachment point to AB;
SG represents a 2-8 oligopeptide group, preferably a dipeptide group or a tripeptide group, or a disulfide, a hydrazone, a glycoside, an acetal or an aminal;
L1, L1′ represent, independently of each other, a straight-chain or branched hydrocarbon chain having 1 to 40 carbon atoms which may be interrupted once or more than once by one or more of —O—, —S—, —SO—, SO₂, —NH—, —CO—, —NMe-, —NHNH—, —SO₂NHNH—, —NHCO—, —CONH—, —CONHNH—, arylene groups, heteroarylene groups, cyclic alkylene groups and 5- to 10-membered heterocyclic groups having up to 4 heteroatoms selected from the group consisting of N, O and S, —SO— or —SO₂—;
   in which * and ^{#} represent the points of attachment of said group with the rest of the compound,
   optionally substituted with one or more substituents selected from the group consisting of halogen, —NHCONH₂, —COOH, —OH, —NH₂, NH—CNNH₂, sulphonamide, sulphone, sulphoxide or sulphonic acid;
L2 represents:

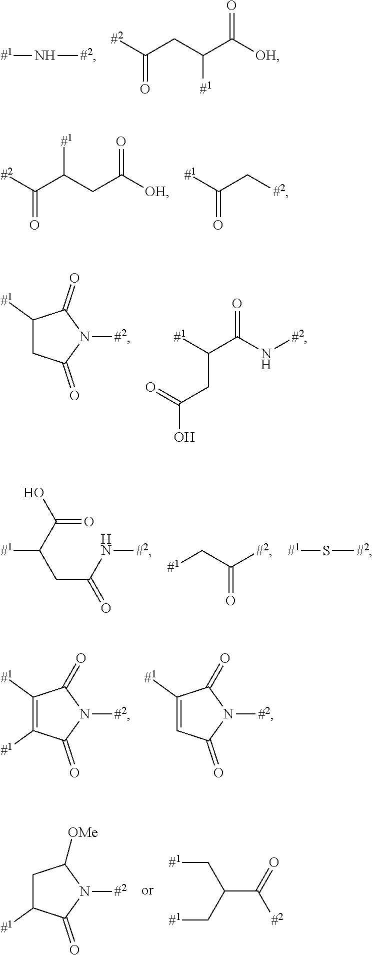

wherein
#¹ represents the attachment point to the binder,
#² represents the attachment point to the group L1, L1′ or SG;
   or the enantiomers, diastereomers, salts, solvates or salts of solvates thereof.

In a further embodiment of the above-mentioned aspects, the invention relates to a conjugate as described supra, wherein L1 and L1′, independently of each other, are those below, where r in each case independently of one another represents a number from 1 to 20, preferably from 1 to 15, particularly preferably from 2 to 20, especially preferably from 2 to 10. It is understood that the groups L1 and L1′ below are read from left to right, meaning that the left-hand symbol [symbol] (or dashed line) in the Table A below denotes the linkage site to §¹-, §¹-L1-SG-, §²- or §²-L1-SG- and the right-hand symbol [symbol] (or dashed line) in the Table A below denotes the linkage site to -SG-L2-§§, -SG-L1′-L2-§§, or -L2-§§.

TABLE A

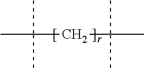

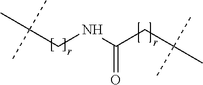

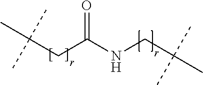

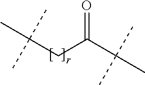

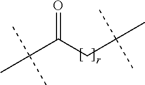

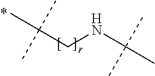

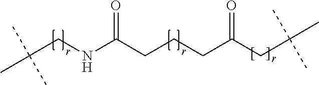

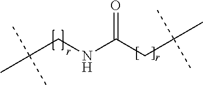

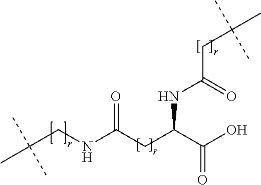

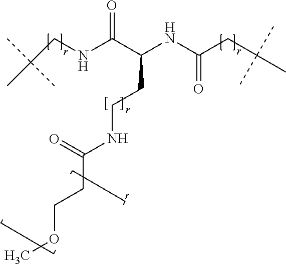

Further Examples of a linker moiety L1 and L1′, independently of each other, are given in the Table below. It is understood that the groups L1 or L1′ below are read from left to right, meaning that the left-hand symbol [symbol] in the Table B below denotes the linkage site to §¹-, §¹-L1-SG-, §²- or §²-L1-SG- and the right-hand symbol [symbol] in the Table B below denotes the linkage site to -SG-L2-§§, -SG-L1′-L2-§§, or -L2-§§.

TABLE B

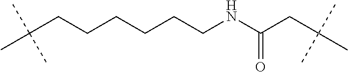

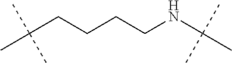

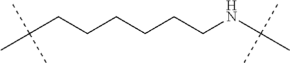

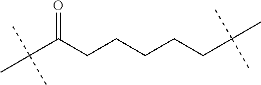

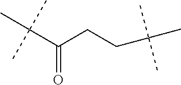

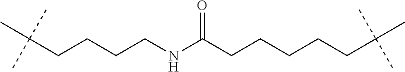

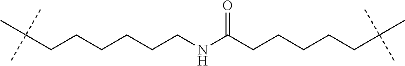

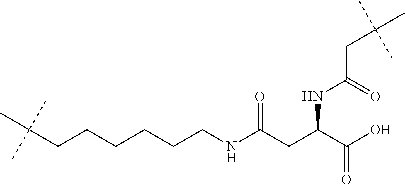

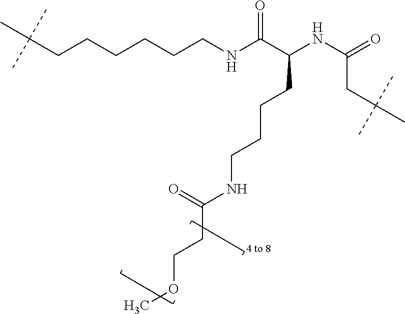

In a further embodiment of the above-mentioned aspects, the invention relates to a conjugate as described supra,

wherein SG comprises 2-6 amino acids selected from the group comprising:
alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, citrulline and valine.

In a further embodiment of the above-mentioned aspects, the invention relates to a conjugate as described supra,

wherein SG comprises 2-3 amino acids selected from the group comprising:
alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, citrulline and valine.

In a further embodiment of the above-mentioned aspects, the invention relates to a conjugate as described supra,

wherein SG comprises 2-3 amino acids selected from the group comprising:
alanine, glycine, histidine, isoleucine, leucine, methionine, serine, citrulline and valine.

In a further embodiment of the above-mentioned aspects, the invention relates to a conjugate as described supra,

wherein SG comprises 2 amino acids selected from the group comprising:
alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, citrulline and valine.

In a further embodiment of the above-mentioned aspects, the invention relates to a conjugate as described supra,

wherein SG comprises 2 amino acids selected from the group comprising:
alanine, glycine, histidine, isoleucine, leucine, methionine, serine, citrulline and valine.

The table C below provides examples showing preferred combinations of linker Z′:
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²-L1-L2-§§
   where r in each case independently of one another represents a number from 1 to 15, preferably from 1 to 10, particularly preferably from 1 to 8. It is understood that the groups L1 and L1′ below are read from left to right, meaning that the left-hand symbol [symbol] in the Table C below denotes the linkage site to §¹-, §¹-L1-SG-, §²- or §²-L1-SG-, respectively, and the right-hand symbol [symbol] in the Table C below denotes the linkage site to -SG-L1′-L2-§§, -SG-L1′-L2-§§ or -L2-§§. It is understood that SG and L1′ are optionally present.

TABLE C

| L1 | SG | L1′ | L2 | | |
|---|---|---|---|---|---|
| 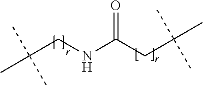 | | | 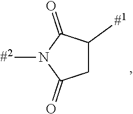 | 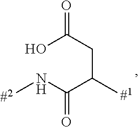 | 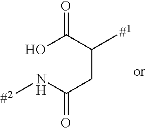 |
| | | | 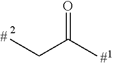 | | |
| 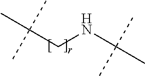 | (C-terminus)- Ala-Val-(N- terminus) | 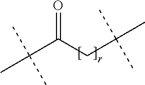 | 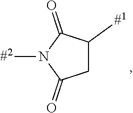 | 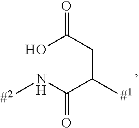 | 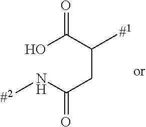 |
| | | | 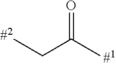 | | |
| 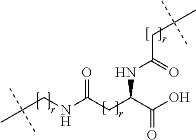 | | | 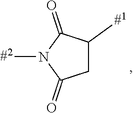 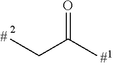 | 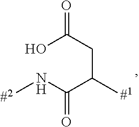 | 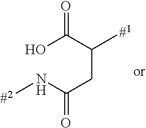 |
| 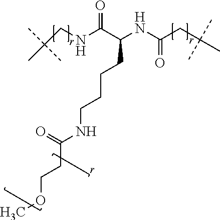 | | | 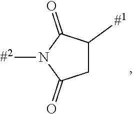 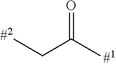 | 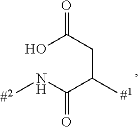 | 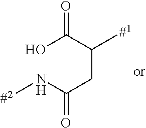 |

wherein for the group L2
#¹ represents the attachment point to the binder,
#² represents the attachment point to the group L1, L1′ or SG.

The table D below provides examples showing more preferred combinations of linker Z′:
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²_L1-L2-§§
   where r in each case independently of one another represents a number from 1 to 15, preferably from 1 to 10, particularly preferably from 1 to 8. It is understood that the groups L1 and L1′ below are read from left to right, meaning that the left-hand symbol [symbol] in the Table D below denotes the linkage site to §¹-, §¹-L1-SG-, §²- or §²-L1-SG-, respectively, and the right-hand symbol [symbol] in the Table D below denotes the linkage site to -SG-L2-§§, -SG-L1′-L2-§§ or -L2-§§.

TABLE D

| Exam- | | | | | | |
|---|---|---|---|---|---|---|
| ple | L1 | SG | L1′ | L2 | | |
| 30, 31, 33, 34, 37III, 38III, 39III, 40III, 46 | 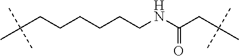 | | | 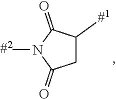 | 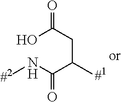 | 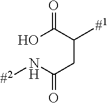 |
| 35, 36 | 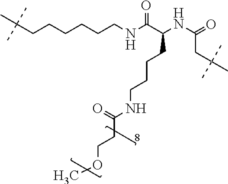 | | | 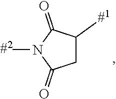 | 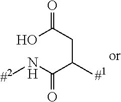 | 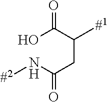 |
| 41, 44 | 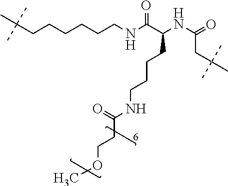 | | | 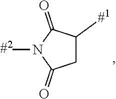 | 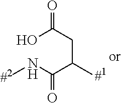 | 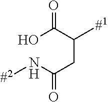 |
| 42 | 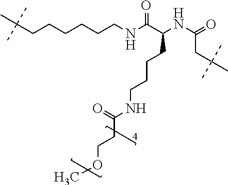 | | | 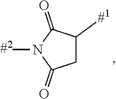 | 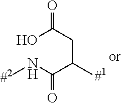 | 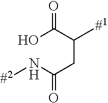 |
| 43 | 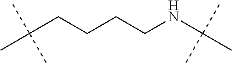 | (C-terminus)- Ala-Val- (N-terminus) | 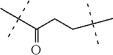 | 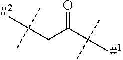 | | |
| 48 | 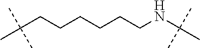 | (C-terminus)- Ala-Val- (N-terminus) | 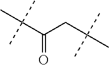 | 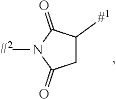 | 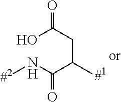 | 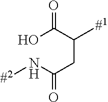 |
| 32 | 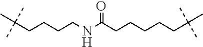 | | | 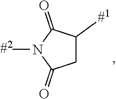 | 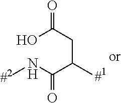 | 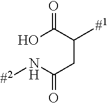 |
| 27, 28 | 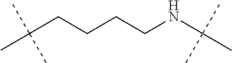 | (C-terminus)- Ala-Val- (N-terminus) | 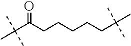 | 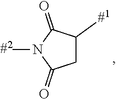 | 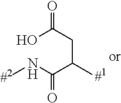 | 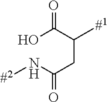 |
| 29 | 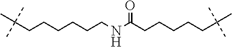 | | | 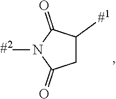 | 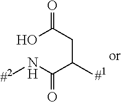 | 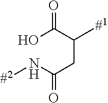 |
| 47 | 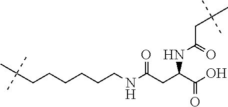 | | | 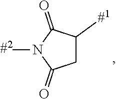 | 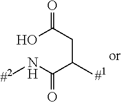 | 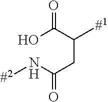 |

wherein for the group L2
#¹ represents the attachment point to the binder,
#² represents the attachment point to the group L1, L1′ or SG.

In a further embodiment of the above-mentioned aspects, the invention relates to a conjugate as described supra,

wherein SG comprises valine and alanine.

In a further embodiment of the above-mentioned aspects, the invention relates to a conjugate as described supra,

wherein SG comprises valine and citrulline.

In a further embodiment of the above-mentioned aspects, the invention relates to a conjugate as described supra,

wherein SG comprises alanine-valine.

In a further embodiment of the above-mentioned aspects, the invention relates to a conjugate as described supra,

wherein SG comprises citrulline-alanine.

In a further embodiment of the above-mentioned aspects, the invention relates to a conjugate as described supra,

wherein SG comprises (C-terminus)-Ala-Val-(N-terminus) or (C-terminus)-Cit-Val-(N-terminus).

In a further embodiment of the above-mentioned aspects, the invention relates to a conjugate as described supra,

wherein SG is (C-terminus)-Ala-Val-(N-terminus) or (C-terminus)-Cit-Val-(N-terminus).

According to another aspect, the invention relates to a conjugate of a binder or a derivative thereof with one or more molecules of an active component, wherein the active component is a NAMPT inhibitor, which is conjugated to the binder via a linker Z′.

In accordance with a further aspect, the invention relates to compounds selected from the group consisting of:
N-{4-[6-Oxo-5-(quinolin-5-yl)-1,4,5,6-tetrahydropyridazin-3-yl]phenyl}-1,3-dihydro-2H-pyrrolo[3,4-c]pyridine-2-carboxamide,
tert-Butyl {4-[-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}carbamate,
N-{4-[1-(4-Aminobutyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamate,
N-{4-[1-(6-Aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[6-Oxo-5-(quinolin-5-yl)-1,6-dihydropyridazin-3-yl]phenyl}-1,3-dihydro-2H-pyrrolo[3,4-c]pyridine-2-carboxamide,
N-{4-[1-(4-Aminobutyl)-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[1-(6-Aminohexyl)-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[5-(5-Methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide,
tert-Butyl {4-[3-{4-[(1,3-dihydro-2H-pyrrolo[3,4-c]pyridin-2-ylcarbonyl)amino]phenyl}-5-(5-methyl-1,3,4-oxadiazol-2-yl)-6-oxopyridazin-1(6H)-yl]butyl}carbamate,
N-{4-[1-(4-Aminobutyl)-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
N-{4-[5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}carbamate,
N-{4-[1-(6-Aminohexyl)-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
N-{4-[1-(6-Aminohexyl)-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide,
N-{4-[(5R)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
tert-Butyl {4-[(5R)-3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]butyl}carbamate,
N-{4-[(5R)-1-(4-aminobutyl)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
N-{4-[6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
tert-Butyl {6-[(3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]hexyl}carbamate,
N-{4-[1-(6-Aminohexyl)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
N-[4-(5-{1-[Oxan-2-yl]-1H-indazol-5-yl}-6-oxo-1,6-dihydropyridazin-3-yl)phenyl]-1,3-dihydro-2H-pyrrolo[3,4-c]pyridine-2-carboxamide,
N-{4-[5-(1H-indazol-5-yl)-6-oxo-1,6-dihydropyridazin-3-yl]phenyl}-1,3-dihydro-2H-pyrrolo[3,4-c]pyridine-2-carboxamide,
N-{4-[5-(1H-indazol-5-yl)-6-oxo-1,6-dihydropyridazin-3-yl]phenyl}-1,3-dihydro-2H-pyrrolo[3,4-c]pyridine-2-carboxamide-hydrogen chloride,
N-{4-[5-(1H-indazol-5-yl)-6-oxo-1,4,5,6-tetrahydropyridazin-3-yl]phenyl}-1,3-dihydro-2H-pyrrolo[3,4-c]pyridine-2-carboxamide,
N-[4-(5-{1-[Oxan-2-yl]-1H-indazol-4-yl}-6-oxo-1,6-dihydropyridazin-3-yl)phenyl]-1,3-dihydro-2H-pyrrolo[3,4-c]pyridine-2-carboxamide,
N-{4-[5-(1H-indazol-4-yl)-6-oxo-1,6-dihydropyridazin-3-yl]phenyl}-1,3-dihydro-2H-pyrrolo[3,4-c]pyridine-2-carboxamide,
N-{4-[5-{1-[oxan-2-yl]-1H-indazol-4-yl}-6-oxo-1,4,5,6-tetrahydropyridazin-3-yl]phenyl}-1,3-dihydro-2H-pyrrolo[3,4-c]pyridine-2-carboxamide,
N-{4-[5-(1H-indazol-4-yl)-6-oxo-1,4,5,6-tetrahydropyridazin-3-yl]phenyl}-1,3-dihydro-2H-pyrrolo[3,4-c]pyridine-2-carboxamide,
N-(4-{5-[1-(4-Methylbenzene-1-sulfonyl)-1H-indol-5-yl]-6-oxo-1,6-dihydropyridazin-3-yl}phenyl)-1,3-dihydro-2H-pyrrolo[3,4-c]pyridine-2-carboxamide,
N-{4-[5-(1H-indol-5-yl)-6-oxo-1,6-dihydropyridazin-3-yl]phenyl}-1,3-dihydro-2H-pyrrolo[3,4-c]pyridine-2-carboxamide,
N-{4-[5-(1H-Indol-5-yl)-6-oxo-1,4,5,6-tetrahydropyridazin-3-yl]phenyl}-1,3-dihydro-2H-pyrrolo[3,4-c]pyridine-2-carboxamide,
N-{4-[5-{1-[Oxan-2-yl]-1H-indazol-5-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-{1-[Oxan-2-yl]-1H-indazol-4-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[6-Oxo-5-(quinolin-7-yl)-1,6-dihydropyridazin-3-yl]phenyl}-1,3-dihydro-2H-pyrrolo[3,4-c]pyridine-2-carboxamide,
tert-Butyl {4-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]butyl}carbamate,
N-{4-[1-(4-Aminobutyl)-6-oxo-5-(quinolin-7-yl)-1,6-dihydropyridazin-3-yl]phenyl}-1,3-dihydro-2H-pyrrolo[3,4-c]pyridine-2-carboxamide,
N-(4-{5-[1-(4-Methylbenzene-1-sulfonyl)-1H-benzimidazol-4-yl]-6-oxo-1,6-dihydropyridazin-3-yl}phenyl)-1,3-dihydro-2H-pyrrolo[3,4-c]pyridine-2-carboxamide,
N-{4-[5-(1H-Benzimidazol-4-yl)-6-oxo-1,6-dihydropyridazin-3-yl]phenyl}-1,3-dihydro-2H-pyrrolo[3,4-c]pyridine-2-carboxamide,
tert-Butyl [(2S)-1-({4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}amino)-1-oxopropan-2-yl]carbamate,
[(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]ammonium trifluoroacetate,
N-(tert-Butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide,
[(1S)-1-[[(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]carbamoyl]-2-methyl-propyl]ammonium trifluoroacetate,
tert-Butyl [(2S)-1-({4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}amino)-1-oxopropan-2-yl]carbamate,
[(1S)-2-[4-[3-[4-(1,3-Dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]ammonium trifluoroacetate,
N-(tert-Butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide,
[(1S)-1-[[(1S)-2-[4-[3-[4-(1,3-Dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]carbamoyl]-2-methyl-propyl]ammonium trifluoroacetate,
2 or 3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic acid,
(9H-fluoren-9-yl)methyl {(32S)-40-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]-26,33-dioxo-2,5,8,11,14,17,20,23-octaoxa-27,34-diazatetracontan-32-yl}carbamate,
N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,6-dihydropyridazin-3-yl]phenyl}-1,3-dihydro-2H-pyrrolo[3,4-c]pyridine-2-carboxamide,
(9H-fluoren-9-yl)methyl {(32S)-40-[(5S)-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-26,33-dioxo-2,5,8,11,14,17,20,23-octaoxa-27,34-diazatetracontan-32-yl}carbamate,
N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide,
(9H-Fluoren-9-yl)methyl {(26S)-34-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-20,27-dioxo-2,5,8,11,14,17-hexaoxa-21,28-diazatetratriacontan-26-yl}carbamate,
N-{4-[6-oxo-1-(6-{[N⁶-(20-oxo-2,5,8,11,14,17-hexaoxaicosan-20-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide,
(9H-Fluoren-9-yl)methyl {(20S)-28-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-14,21-dioxo-2,5,8,11-tetraoxa-15,22-diazaoctacosan-20-yl}carbamate,
N-{4-[6-oxo-1-(6-{[N⁶-(14-oxo-2,5,8,11-tetraoxatetradecan-14-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]hexyl}carbamate,
N-{4-[1-(6-Aminohexyl)-6-oxo-5-(quinolin-7-yl)-1,6-dihydropyridazin-3-yl]phenyl}-1,3-dihydro-2H-pyrrolo[3,4-c]pyridine-2-carboxamide,
N-{4-[1-(6-Aminohexyl)-6-oxo-5-(quinolin-7-yl)-1,6-dihydropyridazin-3-yl]phenyl}-1,3-dihydro-2H-pyrrolo[3,4-c]pyridine-2-carboxamide-hydrogen chloride
(9H-Fluoren-9-yl)methyl {(26S)-34-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]-20,27-dioxo-2,5,8,11,14,17-hexaoxa-21,28-diazatetratriacontan-26-yl}carbamate,
N-{4-[6-oxo-1-(6-{[N⁶-(20-oxo-2,5,8,11,14,17-hexaoxaicosan-20-yl)-L-lysyl]amino}hexyl)-5-(quinolin-7-yl)-1,6-dihydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}carbamate,
N-{4-[1-(6-Aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
N-{4-[1-(6-Aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide hydrogen chloride,
tert-Butyl N²-(tert-butoxycarbonyl)-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparaginate,
N-{6-[3-{4-[(1,3-Dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparagine,
tert-Butyl [(2S)-1-({6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}amino)-1-oxopropan-2-yl]carbamate,
N-{4-[1-[6-(L-Alanylamino)hexyl]-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
N-(tert-Butoxycarbonyl)-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide,
L-Valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide,
N-[6-(2,5-Dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide,
N-[6-(2,5-dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide,
N-{4-[1-(6-{[6-(2,5-Dioxo-2,5-dihydro-1H-pyrrol-1-yl)hexanoyl]amino}hexyl)-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[1-{6-[2-(2,5-Dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[1-(4-{[6-(2,5-dioxo-2,5-dihydro-1H-pyrrol-1-yl)hexanoyl]amino}butyl)-6-oxo-5-(quinolin-7-yl)-1,6-dihydropyridazin-3-yl]phenyl}-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
N-(4-{1-[6-({N²-[(2,5-Dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,6-dihydropyridazin-3-yl}phenyl)-1,3-dihydro-2H-pyrrolo[3,4-c]pyridine-2-carboxamide,
N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[1-[6-({N²-[(2,5-Dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[1-[6-({N²-[(2,5-dioxo-2,5-dihydro-1H-pyrrol-1-yl)acetyl]-N⁶-(14-oxo-2,5,8,11-tetraoxatetradecan-14-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{5-[(2,5-Dioxopyrrolidin-1-yl)oxy]-5-oxopentanoyl}-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide,
N-(4-{1-[6-({N²-[(2,5-dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-7-yl)-1,6-dihydropyridazin-3-yl}phenyl)-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
N-{6-[3-{4-[(1,3-Dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-N²-[(2,5-dioxo-2,5-dihydro-1H-pyrrol-1-yl)acetyl]-D-asparagine,
N-[(2,5-dioxo-2,5-dihydro-1H-pyrrol-1-yl)acetyl]-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide,
tert-Butyl (6-{5-[6-{4-[(1,3-dihydro-2H-pyrrolo[3,4-c]pyridine-2-carbonyl)amino]phenyl}-3-oxo-2-(2,2,2-trifluoroethyl)-2,3-dihydropyridazin-4-yl]-1H-indazol-1-yl}hexyl)carbamate,
N-(4-{5-[1-(6-Aminohexyl)-1H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
N-(4-{5-[2-(6-aminohexyl)-2H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
tert-Butyl (6-{4-[6-{4-[(1,3-dihydro-2H-pyrrolo[3,4-c]pyridin-2-ylcarbonyl)amino]phenyl}-3-oxo-2-(2,2,2-trifluoroethyl)-2,3-dihydropyridazin-4-yl]-1H-indazol-1-yl}hexyl)carbamate,
N-(4-{5-[1-(6-Aminohexyl)-1H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
N-(4-{5-[2-(6-Aminohexyl)-2H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(2-{6-[2-(2,5-dioxo-2,5-dihydro-1H-pyrrol-1-yl)acetamido]hexyl}-2H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(1-{6-[2-(2,5-dioxo-2,5-dihydro-1H-pyrrol-1-yl)acetamido]hexyl}-1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide, and
N-{4-[1-{6-[2-(2,5-dioxo-2,5-dihydro-1H-pyrrol-1-yl)acetamido]hexyl}-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
   or an N-oxide, a salt, a tautomer or a stereoisomer of said compound, or a salt of said N-oxide, tautomer or stereoisomer.

In a further aspect of the present invention, one or more of these compounds may be used as an intermediate to provide a conjugate as described supra.

In another aspect of the present invention, one or more of these compounds may be a metabolite obtainable by the cleavage of a conjugate as described supra.

In accordance with a further aspect, the invention relates to a conjugate as described supra, which is selected from the group consisting of:

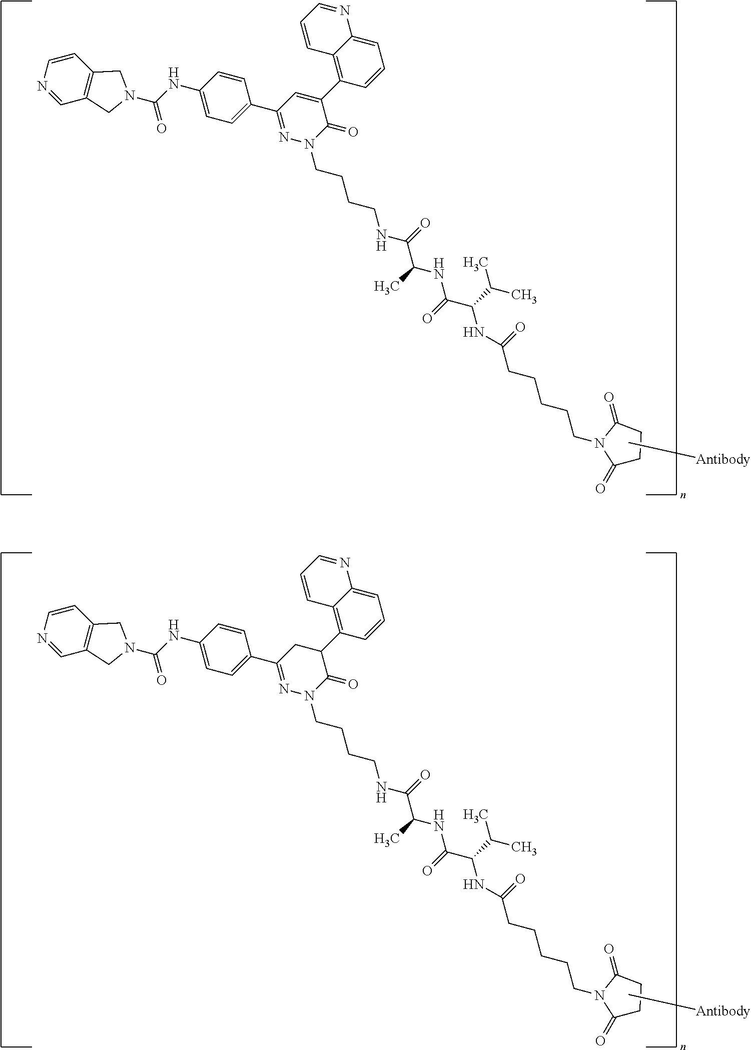

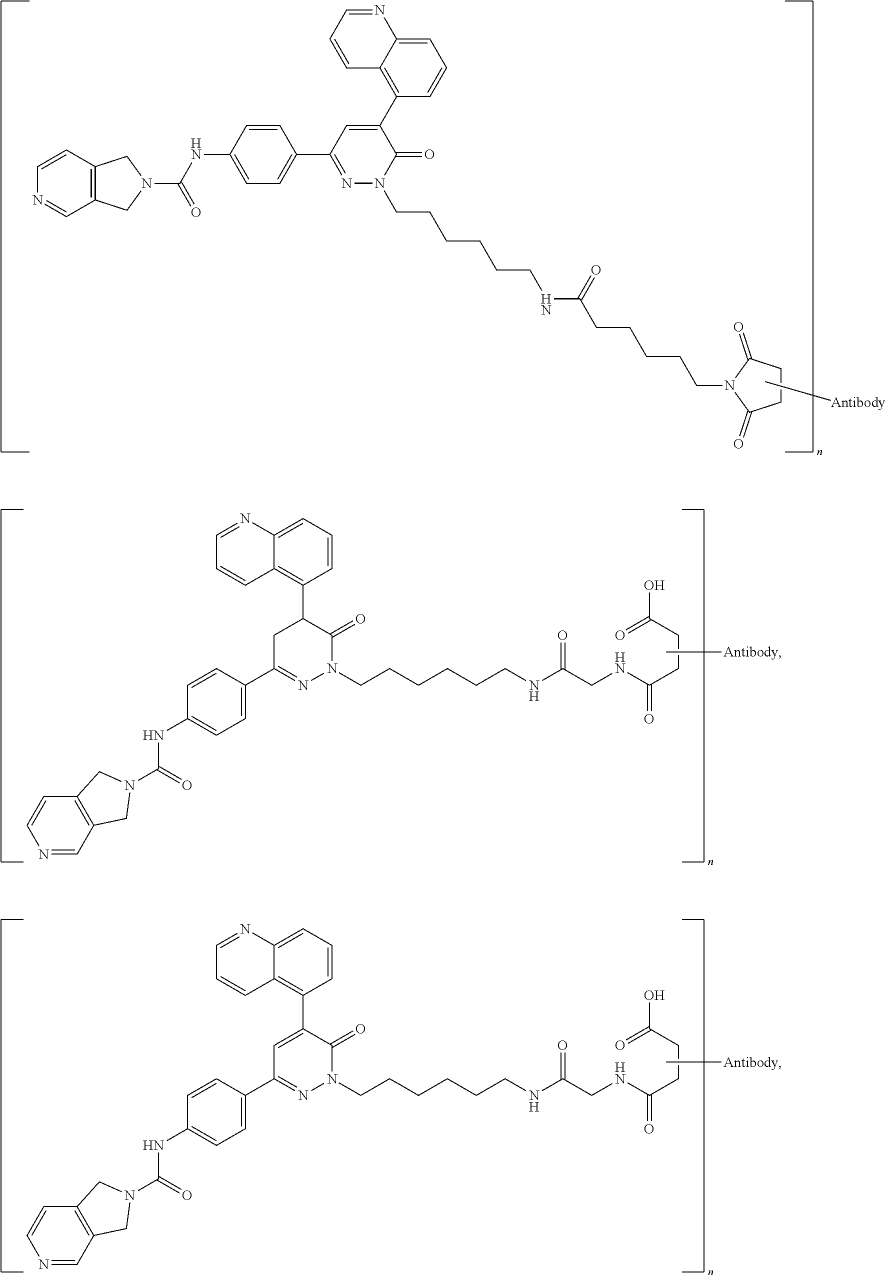

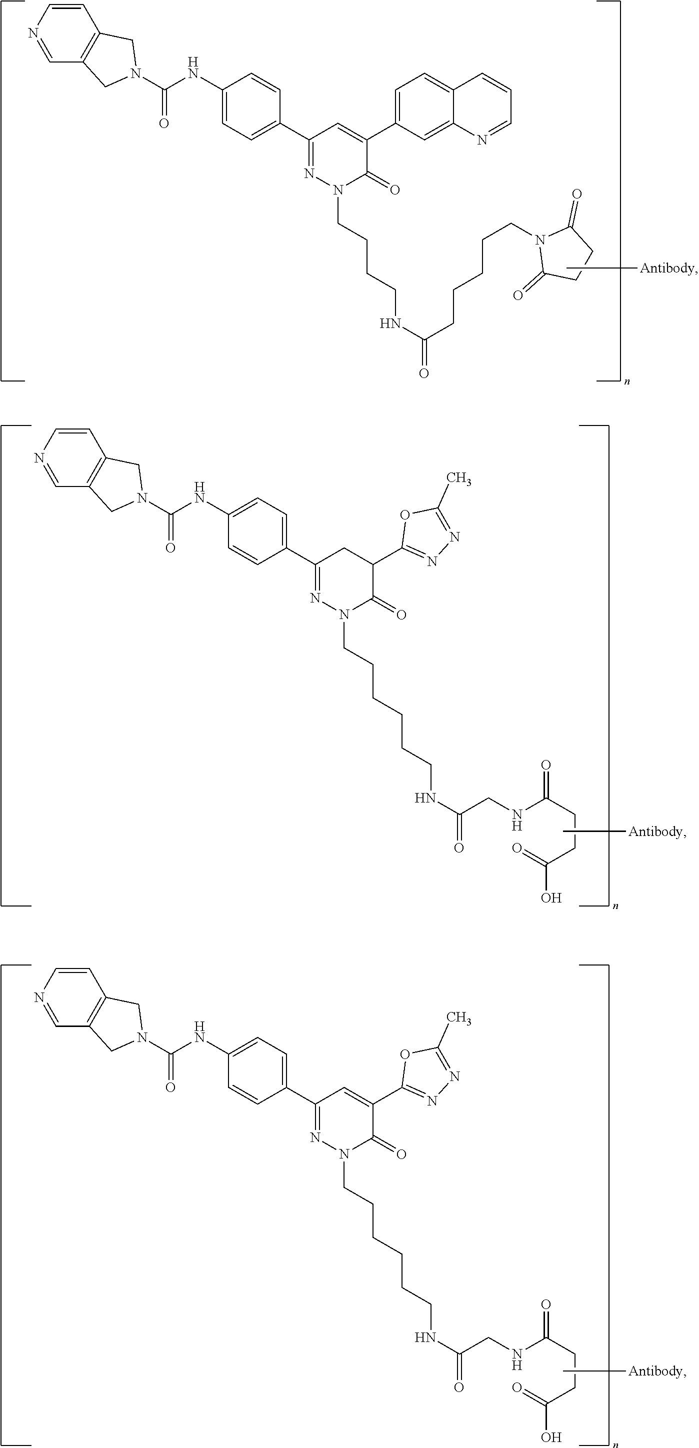

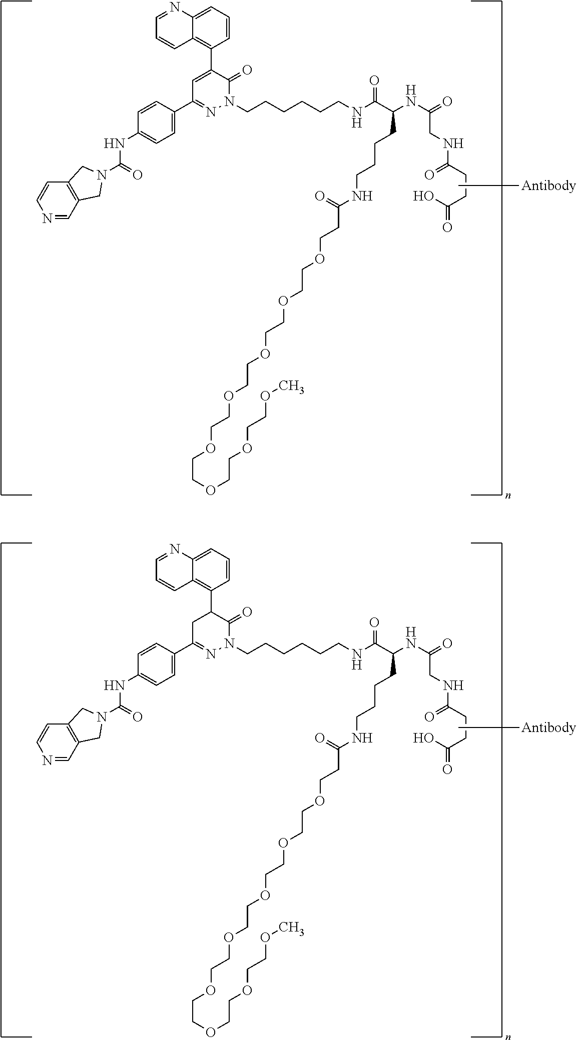

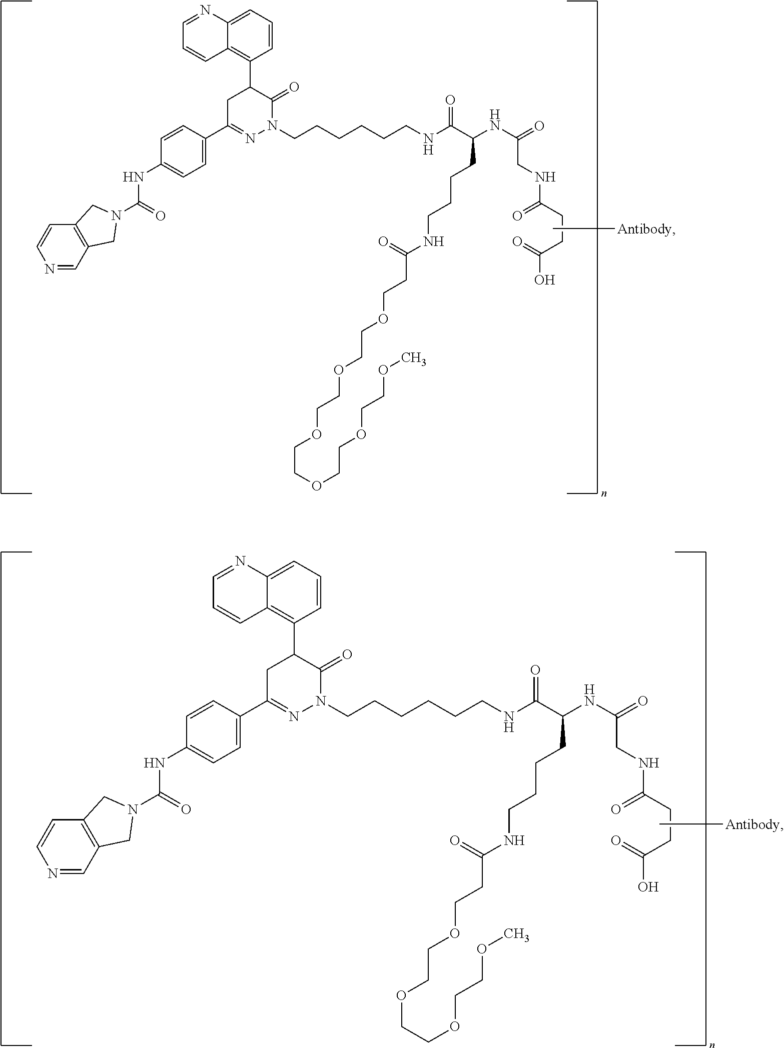

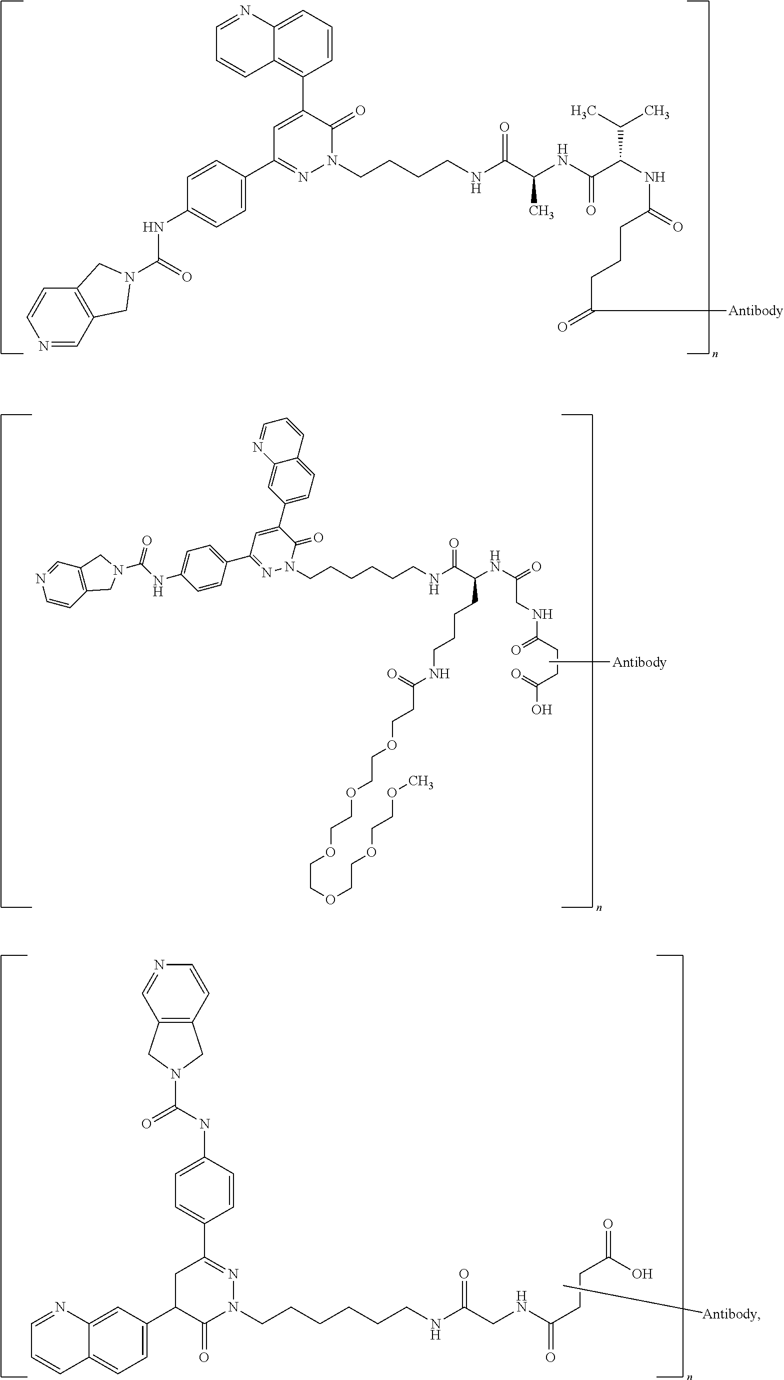

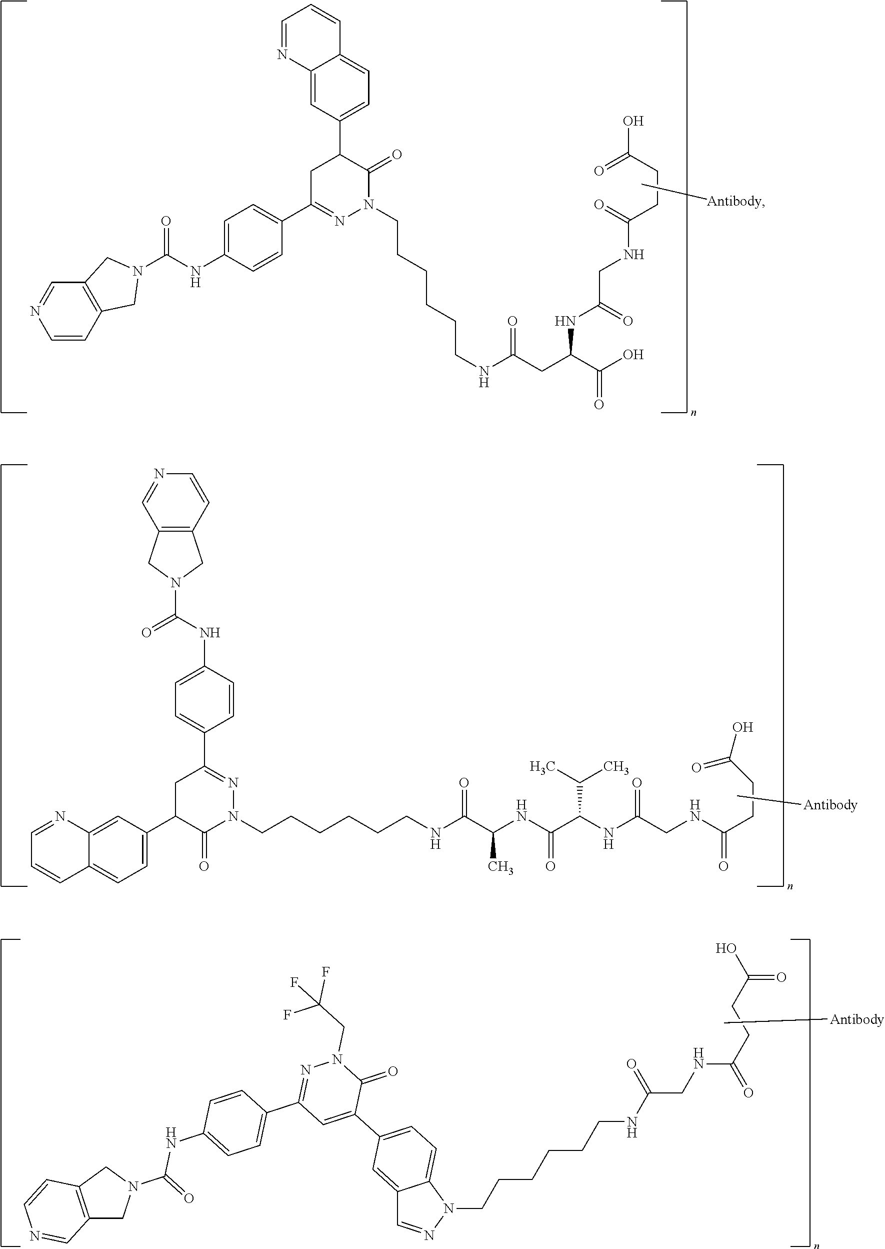

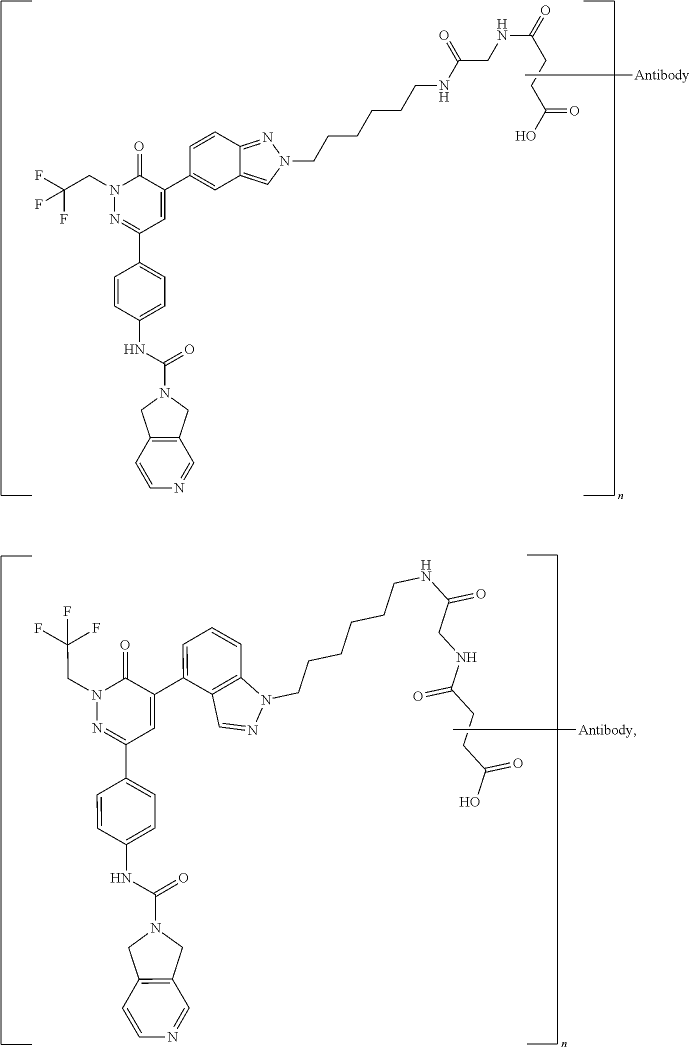

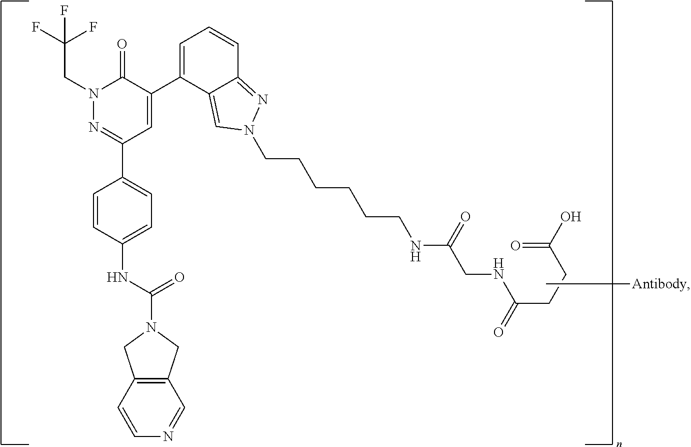

wherein n is a number from 1 to 50, and the antibody is a human, humanized or chimeric monoclonal antibody or an antigen-binding fragment thereof, in particular an anti-HER2-antibody, an anti-CXCR5-antibody, an anti-B7H3-antibody, an anti-C4.4a-antibody, or an antigen binding fragment thereof.

### NAMPT Inhibitor—Linker-Intermediates and Preparation of the Conjugates

The conjugates according to the invention are prepared by initially providing the low-molecular weight NAMPT inhibitor with a linker. The intermediate obtained in this manner is then reacted with the binder (preferably antibody).

The NAMPT inhibitor-linker-intermediates are conjugates of general formula (II-DL) or (III-DL):

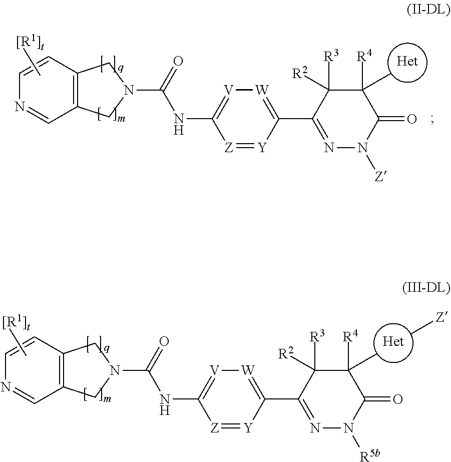

wherein Z′ stands for a linker as defined herein or in the claims and R¹, R², R³, R⁴, R^{5b}, Het, t, q, m, V, W, Z and Y are as defined herein or as defined in any one of claims 1 to 4.

Preferably, for coupling to a cysteine residue, one of the conjugates 1-51 or 1-52 is reacted with the cysteine-containing binder such as an antibody, which is optionally partially reduced for this purpose:

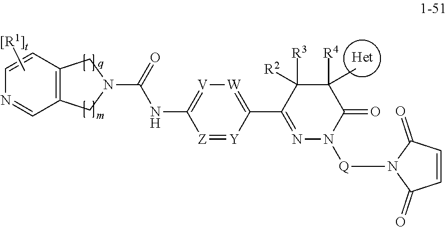

wherein R¹, R², R³, R⁴, Het, t, q, m, V, W, Z and Y are as defined herein or as defined in any one of the claims and Q represents one of the following general structures (i) to (iii):
(i) §-L1-SG-§§
(ii) §-L1-SG-L1′-§§
(iii) §-L1-§§
   wherein
   § represents the attachment point to the pyridazinone ring;
   §§ represents the attachment point to the maleimide group;
   and L1, SG and L1′ are as defined herein or as defined in any one of the claims.

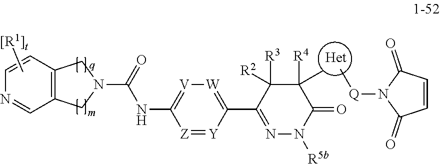

wherein R¹, R², R³, R⁴, R^{5b}, Het, t, q, m, V, W, Z and Y are as defined herein and Q represents one of the following general structures (i) to (iii):
(i) §-L1-SG-§§
(ii) §-L1-SG-L1′-§§
(iii) §-L1-§§
   wherein
   § represents the attachment point to ring Het;
   §§ represents the attachment point to the maleimide group;
   and L1, SG and L1′ are as defined herein or as defined in any one of the claims.

The conjugate may be employed, for example, in the form of its trifluoroacetic acid salt. For the reaction with the binder such as, for example, the antibody, the conjugate is preferably used in a 2- to 20-fold molar excess, preferably in a 5- to 16-fold molar excess with respect to the binder.

For an intermediate coupling to a cysteine residue, the reactions can be illustrated as follows:

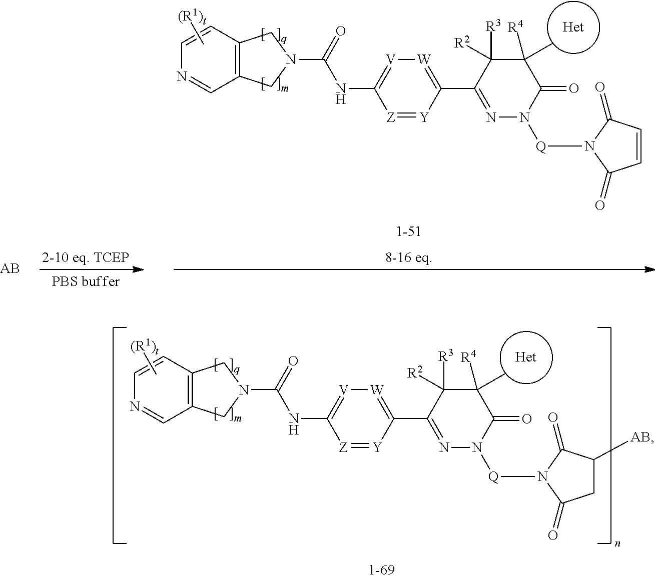

wherein R¹, R², R³, R⁴, Het, t, q, m, V, W, Z, Y and Q are as defined herein; or

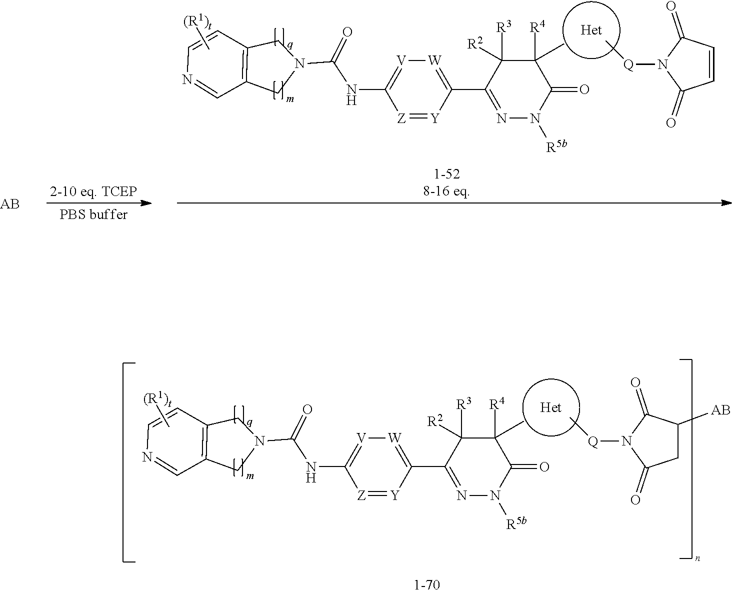

wherein R¹, R², R³, R⁴, R^{5b}, Het, t, q, m, V, W, Z, Y and Q are as defined herein.

Preferably PBS buffer is employed with DMSO, wherein the DMSO does not exceed 10% of the total volume.

In accordance with the invention, this gives preferably conjugates of general formula (II) or (III):

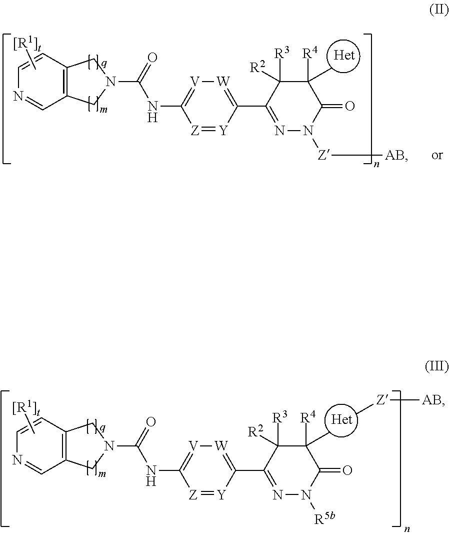

wherein R¹, R², R³, R⁴, R^{5b}, Het, t, q, m, V, W, Z, Y and Q are as defined herein.

AB represents an antibody attached via a cysteine or a lysine residue and n is a number from 1 to 50. With particular preference, AB is a human, humanized or chimeric monoclonal antibody or an antigen-binding fragment thereof, in particular an anti-HER2-antibody, an anti-CXCR5-antibody, an anti-B7H3-antibody, an anti-C4.4a-antibody, or an antigen binding fragment thereof.

Depending on the linker, succinimide-linked ADCs may, after conjugation, be converted into the open-chain succinamides (scheme A1 and scheme A2), which may have an advantageous stability profile.

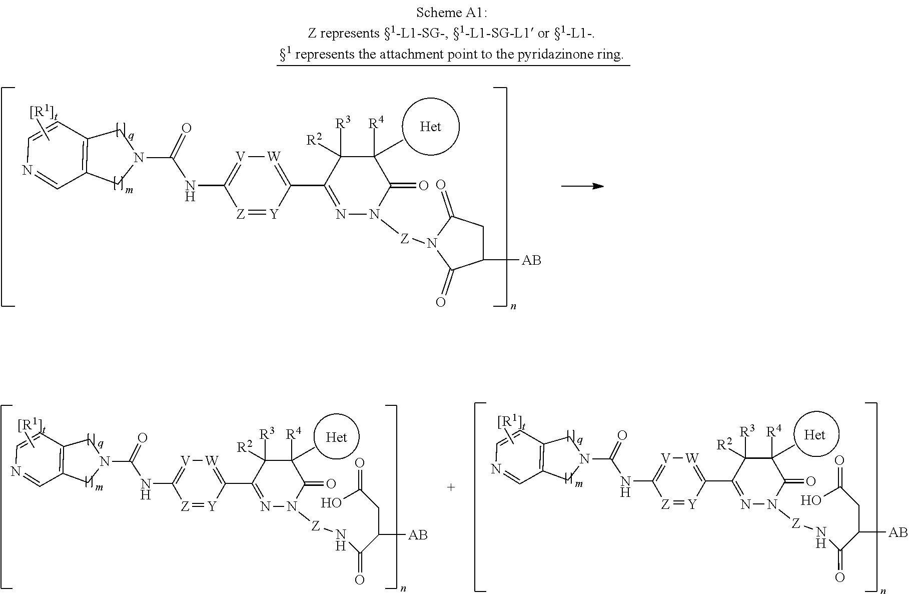

The open-chain succinamides represented in Scheme A1 will be collectively represented as follows:

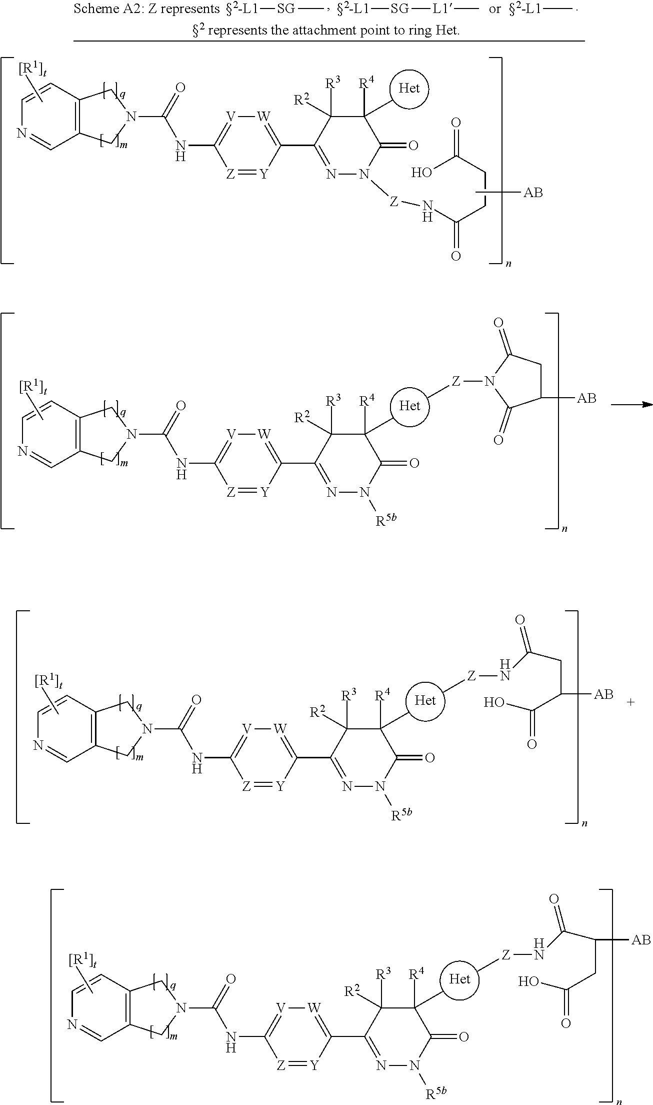

The open-chain succinamides represented in Scheme A2 will be collectively represented as follows:

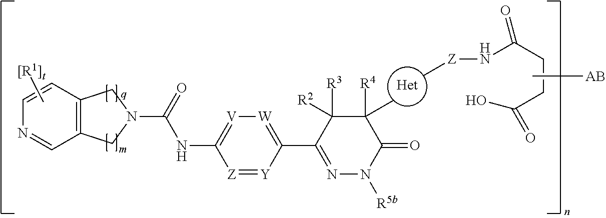

This reaction (ring opening) can be carried out at pH 7.5 to 9, preferably at pH 8, at a temperature of from 20° C. to 37° C., for example by stirring. The preferred stirring time is 8 to 30 hours.

In the above formulae, R¹, R², R³, R⁴, R^{5b}, Het, n, t, q, m, V, W, Z and Y have the same meaning as described herein. AB is an antibody coupled via a cysteine residue or a lysine residue. With particular preference, AB is an anti-HER2-antibody, an anti-CXCR5-antibody, an anti-B7H3-antibody, an anti-C4.4a-antibody, or an antigen binding fragment thereof.

### Binders

In the broadest sense, the term “binder” is understood to mean a molecule which binds to a target molecule present at a certain target cell population to be addressed by the binder/active compound conjugate. The term binder is to be understood in its broadest meaning and also comprises, for example, lectins, proteins capable of binding to certain sugar chains, and phospholipid-binding proteins. Such binders include, for example, high-molecular weight proteins (binding proteins), polypeptides or peptides (binding peptides), non-peptidic (e.g. aptamers (U.S. Pat. No. 5,270,163), review by Keefe A D., et al., Nat. Rev. Drug) Discov. 2010; 9:537-550), or vitamins) and all other cell-binding molecules or substances. Binding proteins are, for example, antibodies and antibody fragments or antibody mimetics such as, for example, affibodies, adnectins, anticalins, DARPins, avimers, nanobodies (review by Gebauer M. et al., Curr. Opinion in Chem. Biol. 2009; 13:245-255; Nuttall S. D. et al., Curr. Opinion in Pharmacology 2008; 8:608-617). Binding peptides are, for example, ligands of a ligand/receptor pair such as, for example, VEGF of the ligand/receptor pair VEGF/KDR, such as transferrin of the ligand/receptor pair transferrin/transferrin receptor or cytokine/cytokine receptor, such as TNFalpha of the ligand/receptor pair TNFalpha/TNFalpha receptor.

The literature also discloses various options of covalent coupling (conjugation) of organic molecules to antibodies. Preference according to the invention is given to the conjugation of the toxophores to the antibody via one or more sulphur atoms of cysteine residues of the antibody and/or via one or more NH groups of lysine residues of the antibody. However, it is also possible to bind the toxophore to the antibody via free carboxyl groups or via sugar residues of the antibody.

In one embodiment of the present invention, the linker Z′ of the conjugate is bound to a cysteine side chain on the binder AB.

The binder or a derivative thereof may be a binding peptide or -protein or a derivative of a binding peptide or -protein.

In a further embodiment of the present invention, each molecule of the active component binds to different amino acids of the binding peptide or -protein or their derivatives respectively, via a linker.

According to one aspect of the present invention, the conjugate averages 1.2 to 50 molecules of the active components per binder.

In accordance with a further aspect of the present invention, the binding peptide or protein represents an antibody or wherein the derivative of the binding peptide or -protein comprises one of the following groups:

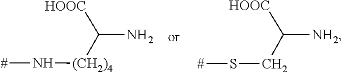

respectively.

In one aspect of the present invention, the binder binds to a cancer target-molecule.

In a further aspect of the present invention, the binder binds to an extracellular target molecule.

After binding to the extracellular target molecule, the binder may be internalized in the expressing cell of the target molecule and is processed intracellularly, preferably through the lysosomal pathway.

In one embodiment the binding peptide or -protein is a human, humanized or chimeric monoclonal antibody, or an antigen-binding fragment thereof.

Preferably the binding peptide or -protein is an anti-HER2-antibody, an anti-CXCR5-antibody, an anti-B7H3-antibody, an anti-C4.4a-antibody, or an antigen binding fragment thereof.

A “target molecule” in the broadest sense is understood to mean a molecule which is present in the target cell population and which may be a protein (for example a receptor of a growth factor) or a non-peptidic molecule (for example a sugar or phospholipid). It is preferably a receptor or an antigen.

The term “extracellular” target molecule describes a target molecule, attached to the cell, which is located at the outside of a cell, or the part of a target molecule which is located at the outside of a cell, i.e. a binder may bind on an intact cell to its extracellular target molecule. An extracellular target molecule may be anchored in the cell membrane or be a component of the cell membrane. The person skilled in the art is aware of methods for identifying extracellular target molecules. For proteins, this may be by determining the transmembrane domain(s) and the orientation of the protein in the membrane. These data are usually deposited in protein databases (e.g. SwissProt).

The term “cancer target molecule” describes a target molecule which is more abundantly present on one or more cancer cell species than on non-cancer cells of the same tissue type. Preferably, the cancer target molecule is selectively present on one or more cancer cell species compared with non-cancer cells of the same tissue type, where selectively describes an at least two-fold enrichment on cancer cells compared to non-cancer cells of the same tissue type (a “selective cancer target molecule”). The use of cancer target molecules allows the selective therapy of cancer cells using the conjugates according to the invention.

The binder can be attached to the linker via a bond. Attachment of the binder can be via a heteroatom of the binder. Heteroatoms according to the invention of the binder which can be used for attachment are sulphur (in one embodiment via a sulphhydryl group of the binder), oxygen (according to the invention by means of a carboxyl or hydroxyl group of the binder) and nitrogen (in one embodiment via a primary or secondary amine group or amide group of the binder). These heteroatoms may be present in the natural binder or are introduced by chemical methods or methods of molecular biology. According to the invention, the attachment of the binder to the toxophore has only a minor effect on the binding activity of the binder with respect to the target molecule. In a preferred embodiment, the attachment has no effect on the binding activity of the binder with respect to the target molecule.

In accordance with the present invention, the term “antibody” is to be understood in its broadest meaning and comprises immunoglobulin molecules, for example intact or modified monoclonal antibodies, polyclonal antibodies or multispecific antibodies (e.g. bispecific antibodies). An immunoglobulin molecule preferably comprises a molecule having four polypeptide chains, two heavy chains (H chains) and two light chains (L chains) which are typically linked by disulphide bridges. Each heavy chain comprises a variable domain of the heavy chain (abbreviated VH) and a constant domain of the heavy chain. The constant domain of the heavy chain may, for example, comprise three domains CH1, CH2 and CH3. Each light chain comprises a variable domain (abbreviated VL) and a constant domain. The constant domain of the light chain comprises a domain (abbreviated CL). The VH and VL domains may be subdivided further into regions having hypervariability, also referred to as complementarity determining regions (abbreviated CDR) and regions having low sequence variability (framework region, abbreviated FR). Typically, each VH and VL region is composed of three CDRs and up to four FRs. For example from the amino terminus to the carboxy terminus in the following order: FR1, CDR1, FR2, CDR2, FR3, CDR3, FR4. An antibody may be obtained from any suitable species, e.g. rabbit, llama, camel, mouse or rat. In one embodiment, the antibody is of human or murine origin. An antibody may, for example, be human, humanized or chimeric.

The term “monoclonal” antibody refers to antibodies obtained from a population of substantially homogeneous antibodies, i.e. individual antibodies of the population are identical except for naturally occurring mutations, of which there may be a small number. Monoclonal antibodies recognize a single antigenic binding site with high specificity. The term monoclonal antibody does not refer to a particular preparation process.

The term “intact” antibody refers to antibodies comprising both an antigen-binding domain and the constant domain of the light and heavy chain. The constant domain may be a naturally occurring domain or a variant thereof having a number of modified amino acid positions.

The term “modified intact” antibody refers to intact antibodies fused via their amino terminus or carboxy terminus by means of a covalent bond (e.g. a peptide bond) with a further polypeptide or protein not originating from an antibody. Furthermore, antibodies may be modified such that, at defined positions, reactive cysteines are introduced to facilitate coupling to a toxophore (see Junutula et al. Nat Biotechnol. 2008 August; 26(8):925-32).

The term “human” antibody refers to antibodies which can be obtained from a human or which are synthetic human antibodies. A “synthetic” human antibody is an antibody which is partially or entirely obtainable in silico from synthetic sequences based on the analysis of human antibody sequences. A human antibody can be encoded, for example, by a nucleic acid isolated from a library of antibody sequences of human origin. An example of such an antibody can be found in Söderlind et al., Nature Biotech. 2000, 18:853-856.

The term “humanized” or “chimeric” antibody describes antibodies consisting of a non-human and a human portion of the sequence. In these antibodies, part of the sequences of the human immunoglobulin (recipient) are replaced by sequence portions of a non-human immunoglobulin (donor). In many cases, the donor is a murine immunoglobulin. In the case of humanized antibodies, amino acids of the CDR of the recipient are replaced by amino acids of the donor. Sometimes, amino acids of the framework, too, are replaced by corresponding amino acids of the donor. In some cases the humanized antibody contains amino acids present neither in the recipient nor in the donor, which were introduced during the optimization of the antibody. In the case of chimeric antibodies, the variable domains of the donor immunoglobulin are fused with the constant regions of a human antibody.

The term complementarity determining region (CDR) as used herein refers to those amino acids of a variable antibody domain which are required for binding to the antigen. Typically, each variable region has three CDR regions referred to as CDR1, CDR2 and CDR3. Each CDR region may embrace amino acids according to the definition of Kabat and/or amino acids of a hypervariable loop defined according to Chotia. The definition according to Kabat comprises, for example, the region from about amino acid position 24-34 (CDR1), 50-56 (CDR2) and 89-97 (CDR3) of the variable light chain and 31-35 (CDR1), 50-65 (CDR2) and 95-102 (CDR3) of the variable heavy chain (Kabat et al., Sequences of Proteins of Immunological Interest, 5th Ed. Public Health Service, National Institutes of Health, Bethesda, Md. (1991)). The definition according to Chotia comprises, for example, the region from about amino acid position 26-32 (CDR1), 50-52 (CDR2) and 91-96 (CDR3) of the variable light chain and 26-32 (CDR1), 53-55 (CDR2) and 96-101 (CDR3) of the variable heavy chain (Chothia and Lesk; J Mol Biol 196: 901-917 (1987)). In some cases, a CDR may comprise amino acids from a CDR region defined according to Kabat and Chotia. Depending on the amino acid sequence of the constant domain of the heavy chain, antibodies may be categorized into different classes. There are five main classes of intact antibodies: IgA, IgD, IgE, IgG and IgM, and several of these can be divided into further subclasses. (Isotypes), e.g. IgG1, IgG2, IgG3, IgG4, IgA1 and IgA2. The constant domains of the heavy chain, which correspond to the different classes, are referred to as [alpha/a], [delta/6], [epsilon/8], [gamma/γ] and [my/p]. Both the three-dimensional structure and the subunit structure of antibodies are known.

The term “functional fragment” or “antigen-binding antibody fragment” of an antibody/immunoglobulin is defined as a fragment of an antibody/immunoglobulin (e.g. the variable domains of an IgG) which still comprises the antigen binding domains of the antibody/immunoglobulin. The “antigen binding domain” of an antibody typically comprises one or more hypervariable regions of an antibody, for example the CDR, CDR2 and/or CDR3 region. However, the “framework” or “skeleton” region of an antibody may also play a role during binding of the antibody to the antigen. The framework region forms the skeleton of the CDRs. Preferably, the antigen binding domain comprises at least amino acids 4 to 103 of the variable light chain and amino acids 5 to 109 of the variable heavy chain, more preferably amino acids 3 to 107 of the variable light chain and 4 to 111 of the variable heavy chain, particularly preferably the complete variable light and heavy chains, i.e. amino acids 1-109 of the VL and 1 to 113 of the VH (numbering according to WO97/08320).

“Functional fragments” or “antigen-binding antibody fragments” of the invention encompass, non-conclusively, Fab, Fab′, F(ab′)2 and Fv fragments, diabodies, Single Domain Antibodies (DAbs), linear antibodies, individual chains of antibodies (single-chain Fv, abbreviated to scFv); and multispecific antibodies, such as bi and tri-specific antibodies, for example, formed from antibody fragments C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag. Antibodies other than “multispecific” or “multifunctional” antibodies are those having identical binding sites. Multispecific antibodies may be specific for different epitopes of an antigen or may be specific for epitopes of more than one antigen (see, for example WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; or Kostelny et al., 1992, J. Immunol. 148: 1547 1553). An F(ab′)2 or Fab molecule may be constructed such that the number of intermolecular disulphide interactions occurring between the Ch1 and the CL domains can be reduced or else completely prevented.

“Epitopes” refer to protein determinants capable of binding specifically to an immunoglobulin or T cell receptors. Epitopic determinants usually consist of chemically active surface groups of molecules such as amino acids or sugar side chains or combinations thereof, and usually have specific 3-dimensional structural properties and also specific charge properties.

“Functional fragments” or “antigen-binding antibody fragments” may be fused with another polypeptide or protein, not originating from an antibody, via the amino terminus or carboxyl terminus thereof, by means of a covalent bond (e.g. a peptide linkage). Furthermore, antibodies and antigen-binding fragments may be modified by introducing reactive cysteines at defined locations, in order to facilitate coupling to a toxophore (see Junutula et al. Nat Biotechnol. 2008 August; 26(8):925-32).

Polyclonal antibodies can be prepared by methods known to a person of ordinary skill in the art. Monoclonal antibodies may be prepared by methods known to a person of ordinary skill in the art (Köhler and Milstein, Nature, 256, 495-497, 1975). Human and humanized monoclonal antibodies may be prepared by methods known to a person of ordinary skill in the art (Olsson et al., Meth Enzymol. 92, 3-16 or Cabilly et al. U.S. Pat. No. 4,816,567 or Boss et al. U.S. Pat. No. 4,816,397).

A person of ordinary skill in the art is aware of diverse methods for preparing human antibodies and fragments thereof, such as, for example, by means of transgenic mice (N Lonberg and D Huszar, Int Rev Immunol. 1995; 13(1):65-93) or Phage Display Technologien (Clackson et al., Nature. 1991 Aug. 15; 352(6336):624-8). Antibodies of the invention may be obtained from recombinant antibody libraries consisting for example of the amino acid sequences of a multiplicity of antibodies compiled from a large number of healthy volunteers. Antibodies may also be produced by means of known recombinant DNA technologies. The nucleic acid sequence of an antibody can be obtained by routine sequencing or is available from publically accessible databases.

An “isolated” antibody or binder has been purified to remove other constituents of the cell. Contaminating constituents of a cell which may interfere with a diagnostic or therapeutic use are, for example, enzymes, hormones, or other peptidic or non-peptidic constituents of a cell. A preferred antibody or binder is one which has been purified to an extent of more than 95% by weight, relative to the antibody or binder (determined for example by Lowry method, UV-Vis spectroscopy or by SDS capillary gel electrophoresis). Moreover an antibody which has been purified to such an extent that it is possible to determine at least 15 amino acids of the amino terminus or of an internal amino acid sequence, or which has been purified to homogeneity, the homogeneity being determined by SDS-PAGE under reducing or non-reducing conditions (detection may be determined by means of Coomassie Blau staining or preferably by silver coloration). However, an antibody is normally prepared by one or more purification steps.

The term “specific binding” or “binds specifically” refers to an antibody or binder which binds to a predetermined antigen/target molecule. Specific binding of an antibody or binder typically describes an antibody or binder having an affinity of at least 10⁻⁷ M (as Kd value; i.e. preferably those with smaller Kd values than 10⁻⁷ M), with the antibody or binder having an at least two times higher affinity for the predetermined antigen/target molecule than for a non-specific antigen/target molecule (e.g. bovine serum albumin, or casein) which is not the predetermined antigen/target molecule or a closely related antigen/target molecule. The antibodies preferably have an affinity of at least 10⁻⁷ M (as Kd value; in other words preferably those with smaller Kd values than 10⁻⁷ M), preferably of at least 10⁻⁸ M, more preferably in the range from 10⁻⁹ M to 10⁻⁷ M. The Kd values may be determined, for example, by means of surface plasmon resonance spectroscopy.

The antibody-drug conjugates of the invention likewise exhibit affinities in these ranges. The affinity is preferably not substantially affected by the conjugation of the drugs (in general, the affinity is reduced by less than one order of magnitude, in other words, for example, at most from 10⁻⁸ M to 10⁻⁷ M).

The antibodies used in accordance with the invention are also notable preferably for a high selectivity. A high selectivity exists when the antibody of the invention exhibits an affinity for the target protein which is better by a factor of at least 2, preferably by a factor of 5 or more preferably by a factor of 10, than for an independent other antigen, e.g. human serum albumin (the affinity may be determined, for example, by means of surface plasmon resonance spectroscopy).

Furthermore, the antibodies of the invention that are used are preferably cross-reactive. In order to be able to facilitate and better interpret preclinical studies, for example toxicological or activity studies (e.g. in xenograft mice), it is advantageous if the antibody used in accordance with the invention not only binds the human target protein but also binds the species target protein in the species used for the studies. In one embodiment the antibody used in accordance with the invention, in addition to the human target protein, is cross-reactive to the target protein of at least one further species. For toxicological and activity studies it is preferred to use species of the families of rodents, dogs and non-human primates. Preferred rodent species are mouse and rat. Preferred non-human primates are rhesus monkeys, chimpanzees and long-tailed macaques.

In one embodiment the antibody used in accordance with the invention, in addition to the human target protein, is cross-reactive to the target protein of at least one further species selected from the group of species consisting of mouse, rat and long-tailed macaque (Macaca fascicularis). Especially preferred are antibodies used in accordance with the invention which in addition to the human target protein are at least cross-reactive to the mouse target protein. Preference is given to cross-reactive antibodies whose affinity for the target protein of the further non-human species differs by a factor of not more than 50, more particularly by a factor of not more than ten, from the affinity for the human target protein.

### Antibodies Directed Against a Cancer Target Molecule

The target molecule towards which the binder, for example an antibody or an antigen-binding fragment thereof, is directed is preferably a cancer target molecule. The term “cancer target molecule” describes a target molecule which is more abundantly present on one or more cancer cell species than on non-cancer cells of the same tissue type. Preferably, the cancer target molecule is selectively present on one or more cancer cell species compared with non-cancer cells of the same tissue type, where selectively describes an at least two-fold enrichment on cancer cells compared to non-cancer cells of the same tissue type (a “selective cancer target molecule”). The use of cancer target molecules allows the selective therapy of cancer cells using the conjugates according to the invention.

Antibodies which are specific against an antigen, for example cancer cell antigen, can be prepared by a person of ordinary skill in the art by means of methods with which he or she is familiar (such as recombinant expression, for example) or may be acquired commercially (as for example from Merck KGaA, Germany). Examples of known commercially available antibodies in cancer therapy are Erbitux® (cetuximab, Merck KGaA), Avastin® (bevacizumab, Roche) and Herceptin® (trastuzumab, Genentech). Trastuzumab is a recombinant humanized monoclonal antibody of the IgG1kappa type which in a cell-based assay (Kd=5 nM) binds the extracellular domains of the human epidermal growth receptor with high affinity. The antibody is produced recombinantly in CHO cells.

In a preferred embodiment, the target molecule is a selective cancer target molecule.

In a particularly preferred embodiment, the target molecule is a protein.

In one embodiment, the target molecule is an extracellular target molecule. In a preferred embodiment, the extracellular target molecule is a protein.

Cancer target molecules are known to those skilled in the art. Examples of these are listed below.

Examples of applicable cancer target molecules are:

| | | NCBI |
|---|---|---|
| Target | Target full name | Gene ID |
| ACVR1 | activin A receptor type 1, ALK2 | 90 |
| ADAM17 | ADAM metallopeptidase domain 17 | 6868 |
| ADAM9 | ADAM metallopeptidase domain 9 | 8754 |
| ADGRE2 | egf-like module containing, mucin-like, hormone | 30817 |
| | receptor-like 2 | |
| AFP | alpha fetoprotein | 174 |
| ALCAM | activated leukocyte cell adhesion molecule, CD166 | 214 |
| ANPEP | alanyl (membrane) aminopeptidase, cd13 | 290 |
| ANXA1 | annexin Al | 301 |
| ASPH | aspartate beta-hydroxylase | 444 |
| ASTL | astacin-like metallo-endopeptidase (M12 family) | 431705 |
| AXL | AXL receptor tyrosine kinase | 558 |
| BCAN | brevican | 63827 |
| BDNF | brain derived neurotrophic factor | 627 |
| BMPR1B | bone morphogenetic protein receptor type 1B | 658 |
| BSG | basigin (Ok blood group) | 682 |
| C16orf54 | chromosome 16 open reading frame 54 | 283897 |
| CA9 | carbonic anhydrase 9 | 768 |
| CCR3 | C-C motif chemokine receptor 3 | 1232 |
| CCR8 | CKR-L1; chemokine (C-C motif) receptor 8 | 1237 |
| CD180 | CD180 molecule | 4064 |
| CD19 | CD19 molecule | 930 |
| CD22 | CD22 molecule | 933 |
| CD206 | mannose receptor C-type 1 | 4360 |
| CD248 | CD248 molecule | 57124 |
| CD274 | CD274 molecule | 29126 |
| CD276 | CD276 molecule, B7H3 | 80381 |
| CD33 | CD33 molecule | 945 |
| CD37 | CD37 molecule | 951 |
| CD38 | CD38 molecule | 952 |
| CD3D | CD3d molecule | 915 |
| CD3E | CD3e molecule | 916 |
| CD44 | CD44 molecule (Indian blood group) | 960 |
| CD46 | CD46 molecule | 4179 |
| CD48 | CD48 molecule | 962 |
| CD5 | CD5 molecule | 921 |
| CD52 | CD52 molecule | 1043 |
| CD6 | CD6 molecule | 923 |
| CD70 | CD70 molecule | 970 |
| CD72 | CD72 molecule | 971 |
| CD74 | CD74 molecule | 972 |
| CD79A | CD79a molecule | 973 |
| CD79B | CD79b molecule | 974 |
| CD99 | CD99 molecule | 4267 |
| CDH15 | cadherin 15 | 1013 |
| CDH3 | cadherin 3 | 1001 |
| CDH6 | cadherin 6 | 1004 |
| CEACAM5 | carcinoembryonic antigen related cell adhesion molecule 5 | 1048 |
| CEACAM6 | carcinoembryonic antigen related cell adhesion molecule 6 | 4680 |
| CHL1 | cell adhesion molecule L1-like | 10752 |
| CLDN6 | claudin 6 | 9074 |
| CLEC12A | C-type lectin domain family 12 member A | 160364 |
| CLEC14A | C-type lectin domain family 14 member A | 161198 |
| CP | ceruloplasmin (ferroxidase) | 1356 |
| CR2 | complement component 3d receptor 2 | 1380 |
| CSPG4 | chondroitin sulfate proteoglycan 4 | 1464 |
| CT83 | cancer/testis antigen 83 | 203413 |
| CTHRC1 | collagen triple helix repeat containing 1 | 115908 |
| CTLA4 | cytotoxic T-lymphocyte associated protein 4 | 1493 |
| CXCR4 | C-X-C motif chemokine receptor 4 | 7852 |
| CXCR5 | C-X-C motif chemokine receptor 5 | 643 |
| DKK1 | dickkopf WNT signaling pathway inhibitor 1 | 22943 |
| DLL3 | delta like canonical Notch ligand 3 | 10683 |
| DPEP1 | dipeptidase 1 (renal) | 1800 |
| DPEP3 | dipeptidase 3 | 64180 |
| EDNRB | endothelin receptor type B | 1910 |
| EFNA4 | ephrin A4 | 1945 |
| EGFR | epidermal growth factor receptor | 1956 |
| EMP2 | epithelial membrane protein 2 | 2013 |
| ENG | endoglin | 2022 |
| ENPP3 | ectonucleotide pyrophosphatase/phosphodiesterase 3 | 5169 |
| EPCAM | epithelial cell adhesion molecule | 4072 |
| EPHA2 | EPH receptor A2 | 1969 |
| EPHB2 | EPH receptor B2 | 2048 |
| ERBB2 | erb-b2 receptor tyrosine kinase 2, HER2 | 2064 |
| ERBB3 | erb-b2 receptor tyrosine kinase 3 | 2065 |
| F3 | coagulation factor III, tissue factor | 2152 |
| FAP | fibroblast activation protein alpha | 2191 |
| FAT1 | FAT atypical cadherin 1 | 2195 |
| FCGR1A | Fc fragment of IgG receptor Ia | 2209 |
| FCGR2B | Fc fragment of IgG receptor IIb | 2213 |
| FCRL1 | Fc receptor like 1 | 115350 |
| FCRL2 | Fc receptor like 2 | 79368 |
| FCRL5 | Fc receptor like 5 | 83416 |
| FGFR2 | fibroblast growth factor receptor 2 | 2263 |
| FGFR3 | fibroblast growth factor receptor 3 | 2261 |
| FGFR4 | fibroblast growth factor receptor 4 | 2264 |
| FLT1 | fms related tyrosine kinase 1 | 2321 |
| FLT3 | fms related tyrosine kinase 3 | 2322 |
| FLT4 | fms related tyrosine kinase 4 | 2324 |
| FN1 | fibronectin 1 | 2335 |
| FOLH1 | folate hydrolase (prostate-specific membrane antigen) 1 | 2346 |
| FOLR1 | folate receptor 1 | 2348 |
| FOLR2 | folate receptor 2 | 2350 |
| FSHR | follicle stimulating hormone receptor | 2492 |
| FUT3 | fucosyltransferase 3 (Lewis blood group) | 2525 |
| GABRP | gamma-aminobutyric acid (GABA) A receptor, pi | 2568 |
| GD2 | ganglioside GD2 | na |
| GD3 | Ganglioside D3 | na |
| GPA33 | glycoprotein A33 | 10223 |
| GPC1 | glypican 1 | |
| GPC3 | glypican 3 | 2719 |
| GPNMB | glycoprotein nmb | 10457 |
| GRPR | gastrin releasing peptide receptor | 2925 |
| GUCY2C | guanylate cyclase 2C | 2984 |
| HAVCR1 | hepatitis A virus cellular receptor 1 | 26762 |
| HAVCR2 | hepatitis A virus cellular receptor 2 | 84868 |
| HLA-DOB | major histocompatibility complex, class II antigen DOB | 3112 |
| HLA-DRB1 | major histocompatibility complex, class II, DR beta 1 | 3123 |
| HMMR | transient receptor potential cation channel, subfamily M, | 3161 |
| | member 8 | |
| HPN | Hepsin | 3249 |
| HSPA5 | heat shock protein family A (Hsp70) member 5 | 3309 |
| ICAM1 | intercellular adhesion molecule 1, CD54 | 3383 |
| IGF1R | insulin-like growth factor 1 receptor | 3480 |
| IL 1RAP | interleukin 1 receptor accessory protein | 3556 |
| IL2 | interleukin 2 | 3558 |
| IL20RA | interleukin 20 receptor subunit alpha | 53832 |
| IL2RA | interleukin 2 receptor subunit alpha | 3559 |
| IL3RA | interleukin 3 receptor subunit alpha | 3563 |
| IL6 | interleukin 6 | 3569 |
| IL7R | interleukin 7 receptor | 3575 |
| ITGA4 | integrin subunit alpha 4 | 3676 |
| ITGAV | integrin subunit alpha V | 3685 |
| ITGB1 | integrin subunit beta 1 | 3688 |
| ITGB6 | integrin subunit beta 6 | 3694 |
| JAG1 | jagged 1 | 182 |
| JAG2 | jagged 2 | 3714 |
| KAAG1 | kidney associated antigen 1 | 353219 |
| KDR | Vascular endothelial growth factor receptor 2 | 3791 |
| KISS1R | KISS1 receptor | 84634 |
| KIT | KIT proto-oncogene receptor tyrosine kinase | 3815 |
| KLK2 | kallikrein-related peptidase 2 | 3817 |
| KLK3 | kallikrein related peptidase 3 | 354 |
| LAMP1 | lysosomal associated membrane protein 1 | 3916 |
| Lewis Y | Lewis Y antigen | na |
| LGR5 | leucine rich repeat containing G protein-coupled receptor 5 | 8549 |
| LHFPL3 | lipoma HMGIC fusion partner-like 3 | 375612 |
| LRRC15 | leucine rich repeat containing 15 | 131578 |
| LY6E | lymphocyte antigen 6 complex, locus E | 4061 |
| LY6G6D | lymphocyte antigen 6 complex, locus G6D | 58530 |
| LY75 | gamma-aminobutyric acid (GABA) A receptor, pi, CD205 | 4065 |
| LYPD1 | LY6/PLAUR domain containing 1 | 116372 |
| LYPD3 | LY6/PLAUR domain containing 3, C4.4a | 27076 |
| MCAM | melanoma cell adhesion molecule (CD146), MUC18 | 4162 |
| MELTF | melanotransferrin, MFI2 | 4241 |
| MET | MET proto-oncogene, receptor tyrosine kinase | 4233 |
| MMP11 | transient receptor potential cation channel, subfamily M, | 4320 |
| | member 8 | |
| MMP16 | matrix metallopeptidase 16 | 4325 |
| MRC1 | Mannose Receptor, CD206 | 4360 |
| MRC2 | ENDO180; mannose receptor, C type 2 | 9902 |
| MS4A1 | membrane spanning 4-domains A1, CD20 | 931 |
| MSLN | mesothelin | 10232 |
| MST1R | macrophage stimulating 1 receptor, RON | 4486 |
| MTX2 | metaxin 2 | 10651 |
| MUC1 | mucin 1, cell surface associated | 4582 |
| MUC16 | mucin 16, cell surface associated | 94025 |
| NCAM1 | neural cell adhesion molecule 1 | 4684 |
| NECTIN4 | nectin cell adhesion molecule 4, PVRL4 | 81607 |
| NOTCH1 | notch 1 | 4851 |
| NOTCH3 | notch 3 | 4854 |
| NPY1R | neuropeptide Y receptor Y1 | 4886 |
| NPY2R | neuropeptide Y receptor Y2 | 4887 |
| NPY4R | neuropeptide Y receptor Y4 | 5540 |
| NPY5R | neuropeptide Y receptor Y5 | 4889 |
| OR51E2 | olfactory receptor family 51 subfamily E member 2 | 81285 |
| P2RX5 | purinergic receptor P2X 5 | 5026 |
| PDCD1 | programmed cell death 1 | 5133 |
| PDGFRB | platelet derived growth factor receptor beta | 5159 |
| PRLR | prolactin receptor | 5618 |
| PRNP | prion protein | 5621 |
| PROM1 | prominin 1 | 8842 |
| PRR4 | proline rich 4 (lacrimal) | 11272 |
| PSCA | prostate stem cell antigen | 8000 |
| PTK7 | protein tyrosine kinase 7 (inactive) | 5754 |
| PTPRC | protein tyrosine phosphatase, receptor type C | 5788 |
| RNF43 | RING-type E3 ubiquitin transferase | 54894 |
| ROR1 | receptor tyrosine kinase like orphan receptor 1 | 4919 |
| SDC1 | syndecan 1 | 6382 |
| SEMA5B | semaphorin 5B | 54437 |
| Sialyl-Tn | glycoepitope | na |
| SLAMF6 | SLAM family member 6, CD352 | 114836 |
| SLAMF7 | SLAM family member 7 | 57823 |
| SLC16A3 | solute carrier family 16, member 3 (monocarboxylic acid | 9123 |
| | transporter 4) | |
| SLC34A2 | solute carrier family 34 member 2 | 10568 |
| SLC39A6 | solute carrier family 39 member 6 | 25800 |
| SLC44A4 | solute carrier family 44 member 4 | 80736 |
| SLC5A1 | solute carrier family 5 (sodium/glucose cotransporter), | 6523 |
| | member 1 | |
| SLC7A11 | solute carrier family 7, (cationic amino acid transporter, | 23657 |
| | y+ system) member 11 | |
| SLC7A5 | solute carrier family 7 member 5 | 8140 |
| SLITRK6 | SLIT and NTRK like family member 6 | 84189 |
| SPON2 | spondin 2, Mindin | 10417 |
| SSTR1 | somatostatin receptor 1 | 6751 |
| SSTR2 | somatostatin receptor 2 | 6752 |
| SSTR5 | somatostatin receptor 5 | 6755 |
| ST14 | suppressor of tumorigenicity 14 protein, matriptase | 6768 |
| STEAP1 | six transmembrane epithelial antigen of the prostate 1 | 26872 |
| STEAP2 | STEAP2 metalloreductase | 261729 |
| SULF2 | sulfatase 2 | 55959 |
| TACSTD2 | tumor-associated calcium signal transducer 2, TROP2 | 4070 |
| TAX1BP3 | Tax1 (human T-cell leukemia virus type I) binding protein 3 | 30851 |
| TDGF1 | teratocarcinoma-derived growth factor 1, CRIPTO | 6997 |
| TFRC | transferrin receptor, CD71 | 7037 |
| TM45F1 | transmembrane 4 L six family member 1 | 4071 |
| TMEFF2 | transmembrane protein with EGF like and two follistatin | 23671 |
| | like domains 2 | |
| TNC | tenascin C | 3371 |
| TNFRSF10A | TNF receptor superfamily member 10a | 8797 |
| TNFRSF12A | FN14, TWEAKR | 51330 |
| TNFRSF13C | TNF receptor superfamily member 13C, BAFFR | 115650 |
| TNFRSF17 | TNF receptor superfamily member 17, BCMA/CD269 | 608 |
| TNFRSF4 | TNF receptor superfamily member 4 | 7293 |
| TNFRSF8 | TNF receptor superfamily member 8, CD30 | 943 |
| TNFSF11 | RANKL | 8600 |
| TNFSF13B | tumor necrosis factor superfamily member 13b | 10673 |
| TNFSF9 | tumor necrosis factor superfamily member 9 | 8744 |
| TPBG | trophoblast glycoprotein | 7162 |
| TRPM4 | transient receptor potential cation channel subfamily M | 54795 |
| | member 4 | |
| TRPM8 | transient receptor potential cation channel, subfamily M, | 79054 |
| | member 8 | |
| UPK1B | uroplakin 1B | 7348 |
| VCAM1 | vascular cell adhesion molecule 1 | 7412 |
| VEGFA | vascular endothelial growth factor A | 7422 |
| VTCN1 | V-set domain containing T cell activation inhibitor 1, | 79679 |
| | B7H4 | |

In one embodiment the binder is a binding protein. In a preferred embodiment the binder is an antibody, an antigen-binding antibody fragment, a multispecific antibody or an antibody mimetic.

Preferred antibody mimetics are affibodies, adnectins, anticalins, DARPins, avimers, or nanobodies. Preferred multispecific antibodies are bispecific and trispecific antibodies.

In a preferred embodiment the binder is an antibody or an antigen-binding antibody fragment, more preferably an isolated antibody or an isolated antigen-binding antibody fragment.

Preferred antigen-binding antibody fragments are Fab, Fab′, F(ab′)2 and Fv fragments, diabodies, DAbs, linear antibodies and scFv. Particularly preferred are Fab, diabodies and scFv.

In a particularly preferred embodiment the binder is an antibody. Particularly preferred are monoclonal antibodies or antigen-binding antibody fragments thereof. Further particularly preferred are human, humanized or chimeric antibodies or antigen-binding antibody fragments thereof.

Antibodies or antigen-binding antibody fragments which bind cancer target molecules may be prepared by a person of ordinary skill in the art using known processes, such as, for example, chemical synthesis or recombinant expression. Binders for cancer target molecules may be acquired commercially or may be prepared by a person of ordinary skill in the art using known processes, such as, for example, chemical synthesis or recombinant expression. Further processes for preparing antibodies or antigen-binding antibody fragments are described in WO 2007/070538 (see page 22 “Antibodies”). The person skilled in the art knows how processes such as phage display libraries (e.g. Morphosys HuCAL Gold) can be compiled and used for discovering antibodies or antigen-binding antibody fragments (see WO 2007/070538, page 24 ff and AK Example 1 on page 70, AK Example 2 on page 72). Further processes for preparing antibodies that use DNA libraries from B cells are described for example on page 26 (WO 2007/070538). Processes for humanizing antibodies are described on page 30-32 of WO2007070538 and in detail in Queen, et al., Pros. Natl. Acad. Sci. USA 86:10029-10033, 1989 or in WO 90/0786. Furthermore, processes for the recombinant expression of proteins in general and of antibodies in particular are known to the person skilled in the art (see, for example, in Berger and Kimrnel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biology, (F. M. Ausabel et al. [Eds.], Current Protocols, Green Publishing Associates, Inc./John Wiley & Sons, Inc.); Harlow et al., (Monoclonal Antibodies: A Laboratory Manual, Cold Spring Harbor Laboratory Press (1988, Paul [Ed.]); Fundamental Immunology, (Lippincott Williams & Wilkins (1998)); and Harlow, et al., (Using Antibodies: A Laboratory Manual, Cold Spring Harbor Laboratory Press (1998)). The person skilled in the art knows the corresponding vectors, promoters and signal peptides which are necessary for the expression of a protein/antibody. Commonplace processes are also described in WO 2007/070538 on pages 41-45. Processes for preparing an IgG1 antibody are described for example in WO 2007/070538 in Example 6 on page 74 ff. Processes which allow the determination of the internalization of an antibody after binding to its antigen are known to the skilled person and are described for example in WO 2007/070538 on page 80. The person skilled in the art is able to use the processes described in WO 2007/070538 that have been used for preparing carboanhydrase IX (Mn) antibodies in analogy for the preparation of antibodies with different target molecule specificity.

### Anti-EGFR Antibodies

Examples of antibodies which bind the cancer target molecules EGFR are cetuximab (INN number 7906), panitumumab (INN number 8499), nimotuzumab (INN number 8545), “TPP-4030”, and “TPP-5653”. Cetuximab (Drug Bank Accession Number DB00002) is a chimeric anti-EGFR1 antibody which is produced in SP2/0 mouse myeloma cells and is sold by ImClone Systems Inc/Merck KgaA/Bristol-Myers Squibb Co. Cetuximab is indicated for the treatment of metastasizing, EGFR expressing, colorectal carcinoma with wild type K-Ras gene. It has an affinity of 10⁻¹⁰M.

In a preferred embodiment, the anti-EGFR antibodies are selected from the group consisting of cetuximab, panitumumab, nimotuzumab, zalutumumab, necitumumab, matuzumab, RG-716, GT-MAB 5.2-GEX, ISU-101, ABT-806, SYM-004, MR1-1, SC-100, MDX-447, DXL-1218, “TPP-4030”, and “TPP-5653”.

In a particularly preferred embodiment the anti-EGFR antibodies are selected from the group consisting of cetuximab, panitumumab, nimotuzumab, zalutumumab, necitumumab and matuzumab.

The person skilled in the art knows of processes which can be used to prepare further antibodies, from the CDR regions of the abovementioned antibodies by means of sequence variations, these further antibodies having a similar or better affinity and/or specificity for the target molecule.

### Anti-HER2 Antibodies

According to the invention, use is made of an anti-HER2 antibody or an antigen-binding fragment thereof. An example of an antibody binding to the cancer target molecule Her2 is trastuzumab (Genentech). Trastuzumab is a humanized antibody used inter alia for the treatment of breast cancer. TPP-1015 is a variant of Trastuzumab.

The expression “anti-HER2 antibody” or “an antibody which binds specifically to HER2” relates to an antibody which binds the cancer target molecule HER2 (ERBB2, UniProtKB/Swiss-Prot Reference P04626), preferably with an affinity sufficient for a diagnostic and/or therapeutic application. In particular embodiments, the antibody binds with a dissociation constant (KD) of ≤1 μM, ≤100 nM, ≤10 nM, ≤≤1 nM, ≤0.1 nM, ≤0.01 nM, or ≤0.001 nM.

Further examples of antibodies binding to HER2 are, in addition to trastuzumab (INN 7637, CAS No.: RN: 180288-69-1) and Pertuzumab (CAS No.: 380610-27-5), the antibodies disclosed in WO 2009/123894-A2, WO 200/8140603-A2 or in WO 2011/044368-A2. An example of an anti-HER2 conjugate is trastuzumab-emtansine (INN-No. 9295). By reference, these antibodies and antigen-binding fragments thereof are incorporated herein, and they can be used in the context of the present invention.

### Anti-TWEAKR Antibodies

Examples of anti-TWEAKR antibodies and antigen-binding fragments are described in WO 2014/198817 (A1) and WO 2015/189143 (A1). By reference, all antibodies of WO 2014/198817 (A1) and WO 2015/189143 (A1) are hereby incorporated into the description of the present invention, and they can be used in the present invention. The sequences of the antibodies are shown in Table 31 and Table 32 of WO 2014/198817 (A1). Preference is given to antibodies, antigen-binding fragments and variants of the antibodies derived from the antibodies referred to as TPP-2090 and TPP-2658. In a further embodiment, the anti-TWEAKR antibodies or antigen-binding antibody fragments comprise at least one, two or three CDR amino acid sequences of an antibody listed in Table 31 or Table 32 of WO 2014/198817 (A1).

In addition, antibodies which bind to TWEAKR are known to the person skilled in the art, see, for example, WO2009/020933(A2) (e.g. PDL-192) or WO2009140177 (A2) (e.g. B11B036 (P4A8)).

Further examples of anti-TWEAKR antibodies and antigen-binding fragments are ITEM-4 and ITEM-4 derived antibodies. ITEM-4 is an anti-TWEAKR antibody described by Nakayama et al. (Nakayama, et al., 2003, Biochem Biophy Res Comm, 306:819-825). Humanized variants of this antibody based on CDR grafting are described by Zhou et al. (Zhou et al., 2013, J Invest Dermatol. 133(4):1052-62) and in WO 2009/020933. ITEM-4 is a moderately agonistically or agonistically acting anti-TWEAKR antibody.

### Anti-CD123-Antibody

The person skilled in the art is familiar with antibodies binding to CD123.

Sun et al. (Sun et al., 1996, Blood 87(1):83-92) describe the generation and properties of the monoclonal antibody 7G3, which binds to the N-terminal domain of IL-3Rα, CD123. U.S. Pat. No. 6,177,078 (Lopez) relates to the anti-CD123 antibody 7G3. A chimeric variant of this antibody (CSL360) is described in WO 2009/070844, and a humanized version (CSL362) in WO 2012/021934. The sequence of the 7G3 antibody is disclosed in EP2426148.

A further anti-CD123 antibody called 12F1 is disclosed in WO 2013/173820.

### Anti-CXCR5 Antibodies

According to the invention, use is made of anti-CXCR5 antibodies.

The expression “anti-CXCR5 antibody” or “an antibody which binds specifically to CXCR5” relates to an antibody which binds the cancer target molecule CXCR5 (NCBI Reference Sequence: NP_001707.1), preferably with an affinity sufficient for a diagnostic and/or therapeutic application. In particular embodiments, the antibody binds with a dissociation constant (K_{D}) of 1 μM, 100 nM, 10 nM, 1 nM, 0.1 nM, 0.01 nM, or 0.001 nM.

Examples of antibodies and antigen-binding fragments which bind to CXCR5 are known to those skilled in the art and are described, for example, in EP2195023.

The hybridoma cells for the rat antibody RF8B2 (ACC2153) were purchased from DSMZ and the sequence of the antibody was identified by standard methods. TPP-9024 a chimeric variant of this antibody with a point mutation at position 67 (S67F) was prepared. Furthermore, the rat antibody sequence constituted the starting point for the humanized antibodies obtained by CDR grafting into human framework.

These antibodies and antigen-binding fragments can be used in the context of this invention.

Particular preference is given in the context of the present invention to the humanized anti-CXCR5 antibody TPP-9574.

### Anti-B7H3-Antibody

According to the invention, use is made of anti-B7H3 antibodies.

The expression “anti-B7H3 antibody” or “an antibody which binds specifically to B7H3” relates to an antibody which binds the cancer target molecule B7H3 (UniProtKB/Swiss-Prot Reference: Q5ZPR3), preferably with an affinity sufficient for a diagnostic and/or therapeutic application. In particular embodiments, the antibody binds with a dissociation constant (K_{D}) of ≤1 μM, ≤100 nM, ≤≤10 nM, ≤≤1 nM, ≤0.1 nM, ≤0.01 nM, or ≤0.001 nM.

Anti-B7H3 antibodies were generated, for example, by screening of a phage display library for recombinant murine B7H3 (murine CD276; Gene ID: 102657) and human B7H3 (human CD276; Gene ID: 80381) expressing cells. Particularly the antibody TPP-8382 is a preferred example. The antibodies obtained in this manner were reformatted into the human IgG1 format. These two antibodies were used for the working examples described here. In addition, antibodies which bind to B7H3 are known to the person skilled in the art.

Particular preference is given in the context of the present invention to the humanized anti-B7H3 antibody TPP-8382.

Anti-C4.4a-Antibody

According to the invention, use may be made of C4.4a antibodies.

The expression “anti-C4.4a antibody” or “an antibody which binds specifically to C4.4a” relates to an antibody which binds the cancer target molecule C4.4a (LYPD3, UniProtKB/Swiss-Prot Reference: 095274), preferably with an affinity sufficient for a diagnostic and/or therapeutic application. In particular embodiments, the antibody binds with a dissociation constant (K_{D}) of 1 μM, 100 nM, 10 nM, ≤1 nM, 0.1 nM, 0.01 nM, or 0.001 nM.

Examples of anti-C4.4a antibodies and antigen-binding fragments are described in WO 2012/143499 A2. By reference, all antibodies of WO 2012/143499 A2 are hereby incorporated into the description of the present invention, and they can be used in the present invention. The sequences of the antibodies are given in Table 1 of WO 2012/143499 A2, where each row shows the respective CDR amino acid sequences of the variable light chain or the variable heavy chain of the antibody listed in column 1.

In one embodiment, the anti-C4.4a antibodies or antigen-binding antibody fragments thereof are, after binding to a cell expressing C4.4a, internalized by the cell.

In a further embodiment, the anti-C4.4a antibodies or antigen-binding antibody fragments comprise at least one, two or three CDR amino acid sequences of an antibody listed in Table 1 of WO 2012/143499 A2 or Table 2 of WO 2012/143499 A2. Preferred embodiments of such antibodies are likewise listed in WO 2012/143499 A2 and incorporated herein by reference.

Particular preference is given in the context of the present invention to the humanized anti-C4.4a antibodies TPP-509 and TPP-668.

### Antibody Sequences

In the present application, reference is made to the following preferred antibodies shown in this Table:

| | | SEQ ID NO: | | | | | | | | | |
|---|---|---|---|---|---|---|---|---|---|---|---|
| | | | | | | | | | | Heavy | Light |
| Antibody | Target | VH | H-CDR1 | H-CDR2 | H-CDR3 | VL | L-CDR1 | L-CDR2 | L-CDR3 | Chain | Chain |
| TPP-509 | C4.4a | 1 | 2 | 3 | 4 | 5 | 6 | 7 | 8 | 9 | 10 |
| TPP-668 | C4.4a | 11 | 12 | 13 | 14 | 15 | 16 | 17 | 18 | 19 | 20 |
| TPP-1015 | HER2 | 21 | 22 | 23 | 24 | 25 | 26 | 27 | 28 | 29 | 30 |
| (Trastuzumab | | | | | | | | | | | |
| like) | | | | | | | | | | | |
| TPP-8382 | B7H3 | 31 | 32 | 33 | 34 | 35 | 36 | 37 | 38 | 39 | 40 |
| TPP-9574 | CXCR5 | 41 | 42 | 43 | 44 | 45 | 46 | 47 | 48 | 49 | 50 |

TPP-509 is an anti-C4.4a antibody which comprises a variable heavy chain comprising the variable CDR1 sequence of the heavy chain, as shown in SEQ ID NO: 2, the variable CDR2 sequence of the heavy chain, as shown in SEQ ID NO: 3, and the variable CDR3 sequence of the heavy chain, as shown in SEQ ID NO: 4, and

a variable light chain comprising the variable CDR1 sequence of the light chain, as shown in SEQ ID NO: 6, the variable CDR2 sequence of the light chain, as shown in SEQ ID NO: 7, and the variable CDR3 sequence of the light chain, as shown in SEQ ID NO: 8.

TPP-668 is an anti-C4.4a antibody which comprises a variable heavy chain comprising the variable CDR1 sequence of the heavy chain, as shown in SEQ ID NO: 12, the variable CDR2 sequence of the heavy chain, as shown in SEQ ID NO: 13, and the variable CDR3 sequence of the heavy chain, as shown in SEQ ID NO: 14, and

a variable light chain comprising the variable CDR1 sequence of the light chain, as shown in SEQ ID NO: 16, the variable CDR2 sequence of the light chain, as shown in SEQ ID NO: 17, and the variable CDR3 sequence of the light chain, as shown in SEQ ID NO: 18.

TPP-1015 is an anti-HER2 antibody which comprises a variable heavy chain comprising the variable CDR1 sequence of the heavy chain, as shown in SEQ ID NO: 22, the variable CDR2 sequence of the heavy chain, as shown in SEQ ID NO: 23, and the variable CDR3 sequence of the heavy chain, as shown in SEQ ID NO: 24, and

a variable light chain comprising the variable CDR1 sequence of the light chain, as shown in SEQ ID NO: 26, the variable CDR2 sequence of the light chain, as shown in SEQ ID NO: 27, and the variable CDR3 sequence of the light chain, as shown in SEQ ID NO: 28.

TPP-8382 is an anti-B7H3 antibody which comprises a variable heavy chain comprising the variable CDR1 sequence of the heavy chain, as shown in SEQ ID NO: 32, the variable CDR2 sequence of the heavy chain, as shown in SEQ ID NO: 33, and the variable CDR3 sequence of the heavy chain, as shown in SEQ ID NO: 34, and

a variable light chain comprising the variable CDR1 sequence of the light chain, as shown in SEQ ID NO: 36, the variable CDR2 sequence of the light chain, as shown in SEQ ID NO: 37, and the variable CDR3 sequence of the light chain, as shown in SEQ ID NO: 38.

TPP-9574 is an anti-CXCR5 antibody which comprises a variable heavy chain comprising the variable CDR1 sequence of the heavy chain, as shown in SEQ ID NO: 42, the variable CDR2 sequence of the heavy chain, as shown in SEQ ID NO: 43, and the variable CDR3 sequence of the heavy chain, as shown in SEQ ID NO: 44, and

a variable light chain comprising the variable CDR1 sequence of the light chain, as shown in SEQ ID NO: 46, the variable CDR2 sequence of the light chain, as shown in SEQ ID NO: 47, and the variable CDR3 sequence of the light chain, as shown in SEQ ID NO: 48.

TPP-509 is an antibody which comprises preferentially a variable region of the heavy chain (VH) as shown in SEQ ID NO: 1 and a variable region of the light chain (VL) as shown in SEQ ID NO: 5.

TPP-668 is an antibody which comprises preferentially a variable region of the heavy chain (VH) as shown in SEQ ID NO: 11 and a variable region of the light chain (VL) as shown in SEQ ID NO: 15.

TPP-1015 is an antibody which comprises preferentially a variable region of the heavy chain (VH) as shown in SEQ ID NO: 21 and a variable region of the light chain (VL) as shown in SEQ ID NO: 25.

TPP-8382 is an antibody which comprises preferentially a variable region of the heavy chain (VH) as shown in SEQ ID NO: 31 and a variable region of the light chain (VL) as shown in SEQ ID NO: 35.

TPP-9574 is an antibody which comprises preferentially a variable region of the heavy chain (VH) as shown in SEQ ID NO: 41 and a variable region of the light chain (VL) as shown in SEQ ID NO: 45.

TPP-509 is an antibody comprising preferentially a region of the heavy chain as shown in SEQ ID NO: 9 and a region of the light chain as shown in SEQ ID NO: 10.

TPP-668 is an antibody comprising preferentially a region of the heavy chain as shown in SEQ ID NO: 19 and a region of the light chain as shown in SEQ ID NO: 20.

TPP-1015 is an antibody comprising a region of the heavy chain as shown in SEQ ID NO: 29 and a region of the light chain as shown in SEQ ID NO: 30.

TPP-8382 is an antibody comprising preferentially a region of the heavy chain as shown in SEQ ID NO: 39 and a region of the light chain as shown in SEQ ID NO: 40.

TPP-9574 is an antibody comprising preferentially a region of the heavy chain as shown in SEQ ID NO: 49 and a region of the light chain as shown in SEQ ID NO: 50.

### DNA Molecules of the Invention

The present invention also relates to the DNA molecules that encode an antibody of the invention or antigen-binding fragment thereof. These sequences are optimized in certain cases for mammalian expression. DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent DNA Variants

Yet another class of DNA variants within the scope of the invention may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs. Methods of efficiently synthesizing oligonucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present invention further provides recombinant DNA constructs comprising one or more of the nucleotide sequences encoding the preferred antibodies of the present invention. The recombinant constructs of the present invention can be used in connection with a vector, such as a plasmid, phagemid, phage or viral vector, into which a DNA molecule encoding an antibody of the invention or antigen-binding fragment thereof or variant thereof is inserted.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic hosts cells are yeasts, insects and insect cells, plants and plant cells, transgenic animals, or mammalian cells. In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

Therefore, an embodiment of the present invention are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present invention is a method of using the host cell to produce an antibody and antigen binding fragments, comprising culturing the host cell under suitable conditions and recovering said antibody.

Therefore another embodiment of the present invention is the production of the antibodies according to this invention with the host cells of the present invention and purification of these antibodies to at least 95% homogeneity by weight.

### Bacterial Expression

Useful expression vectors for bacterial use are constructed by inserting a DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present invention is an expression vector comprising a nucleic acid sequence encoding for the novel antibodies of the present invention.

Antibodies of the present invention or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Expression of the antibodies may be constitutive or regulated (e.g. inducible by addition or removal of small molecule inductors such as Tetracyclin in conjunction with Tet system). For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and 5,179,017). Suitable selectable markers include genes that confer resistance to drugs such as G418, puromycin, hygromycin, blasticidin, zeocin/bleomycin or methotrexate or selectable marker that exploit auxotrophies such as Glutamine Synthetase (Bebbington et al., Biotechnology (N Y). 1992 February; 10(2):169-75), on a host cell into which the vector has been introduced. For example, the dihydrofolate reductase (DHFR) gene confers resistance to methotrexate, neo gene confers resistance to G418, the bsd gene from Aspergillus terreus confers resistance to blasticidin, puromycin N-acetyl-transferase confers resistance to puromycin, the Sh ble gene product confers resistance to zeocin, and resistance to hygromycin is conferred by the E. coli hygromycin resistance gene (hyg or hph). Selectable markers like DHFR or Glutamine Synthetase are also useful for amplification techniques in conjunction with MTX and MSX.

Transfection of the expression vector into a host cell can be carried out using standard techniques such as electroporation, nucleofection, calcium-phosphate precipitation, lipofection, polycation-based transfection such as polyethlylenimine (PEI)-based transfection and DEAE-dextran transfection.

Suitable mammalian host cells for expressing the antibodies, antigen binding fragments thereof or variants thereof provided herein include Chinese Hamster Ovary (CHO cells) such as CHO-K1, CHO-S, CHO-K1SV [including dhfr-CHO cells, described in Urlaub and Chasin, (1980) Proc. Natl. Acad. Sci. USA 77:4216-4220 and Urlaub et al., Cell. 1983 June; 33(2):405-12, used with a DHFR selectable marker, e.g., as described in R. J. Kaufman and P. A. Sharp (1982) Mol. Biol. 159:601-621; and other knockout cells exemplified in Fan et al., Biotechnol Bioeng. 2012 April; 109(4):1007-15], NS0 myeloma cells, COS cells, HEK293 cells, HKB11 cells, BHK21 cells, CAP cells, EB66 cells, and SP2 cells.

Expression might also be transient or semi-stable in expression systems such as HEK293, HEK293T, HEK293-EBNA, HEK293E, HEK293-6E, HEK293-Freestyle, HKB11, Expi293F, 293EBNALT75, CHO Freestyle, CHO-S, CHO-K1, CHO-K1SV, CHOEBNALT85, CHOS-XE, CHO-3E7 or CAP-T cells (for instance Durocher et al., Nucleic Acids Res. 2002 Jan. 15; 30(2):E9).

In some embodiments, the expression vector is designed such that the expressed protein is secreted into the culture medium in which the host cells are grown. The antibodies, antigen binding fragments thereof or variants thereof can be recovered from the culture medium using standard protein purification methods.

### Purification

Antibodies of the invention or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof or variants thereof include naturally purified products, products of chemical synthetic procedures, and products produced by recombinant techniques from an eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

In preferred embodiments, the antibody is purified (1) to greater than 95% by weight of antibody as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

### Metabolites

Following introduction of the ADCs into tumour cells and subsequent dissociation of the conjugate, a cytotoxic metabolite may be formed that is released within the tumour cell and can unfold its action therein directly and selectively.

For the purpose of the present invention the term metabolite is understood as the product of the (e.g., enzymatic or chemical) cleavage of the conjugate of a binder or a derivative thereof according to the present invention.

The present invention thus also relates to metabolites obtainable by the cleavage of any of the conjugates described herein.

Accordingly, the metabolite will comprise a molecule of an active component, wherein the active component is a NAMPT inhibitor.

In one embodiment, if the conjugate according to the invention comprises a stable linker, the metabolite of the NAMPT inhibitor as such comprises an amino acid residue, preferably a cysteine or a lysine residue of the binder protein or peptide.

In another embodiment, if the conjugate according to the invention comprises a cleavable linker, the metabolite of the NAMPT inhibitor as such is possibly connected with only part of the linker moiety, i.e. the metabolite of the NAMPT inhibitor as such does not comprise a cysteine and/or a lysine residue of the binder protein or peptide.

### General Procedures

The conjugates according to the invention can be prepared according to the following schemes 1 through.

The schemes and procedures described below illustrate synthetic routes to the compounds of formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents, R¹, R², R³, R⁴, R⁵, R⁶, R⁷, R⁸, R⁹, R¹⁰, R¹¹, R¹², R¹³, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3rd edition, Wiley 1999). Specific examples are described in the subsequent paragraphs.

The racemic and chiral synthesis of dihydropyridazinones is described in the following representative patents and journals: WO2011138427; U.S. Pat. No. 4,666,902; US20080027041; EP185964; EP196005; EP175363; EP240026; EP400519; EP344634; DE10010423; WO2001064652; DE10010426; DE10010430; DE2304977; Chem. Pharm. Bull. 46(1), 84-96 (1998); J. Med. Chem. 39, 297-303 (1996); J. Med. Chem. 50, 3242-3255 (2007); Bioorganic & Medicinal Chemistry Letters 21, 5493-5497 (2011).

One route for the preparation of compounds of formula (Ia) is described in Scheme 1.

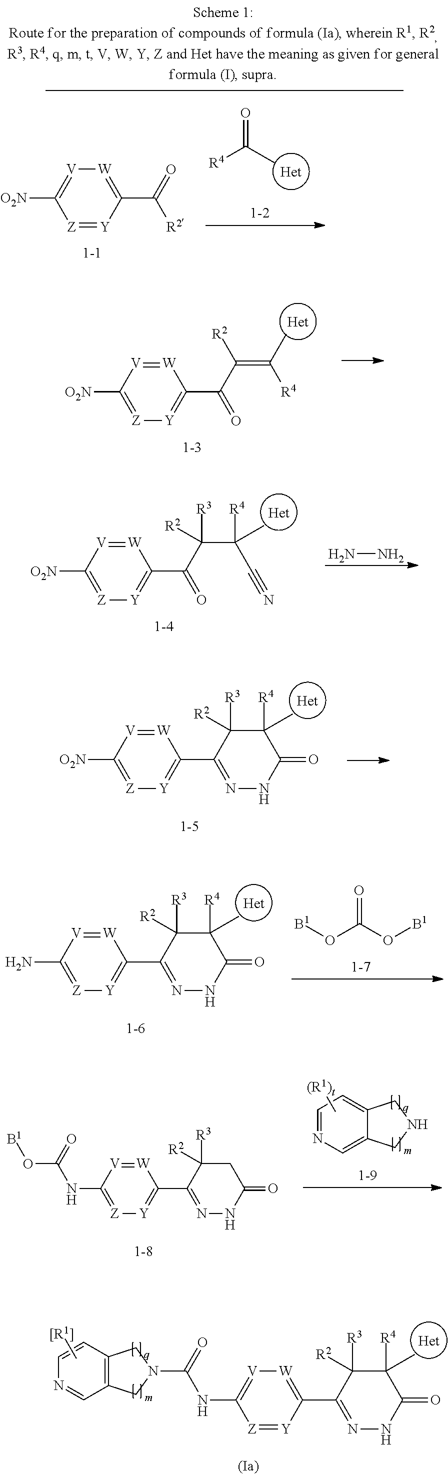

B¹ represents a leaving group such as, for example, a haloalkyl such as, for example, trichloromethyl or a imide such as, for example, pyrrolidine-2,5-dione or p-nitrophenyl.

In addition, interconversion of any of the substituents R¹, R², R³, R⁴, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-1, 1-2, 1-7 and 1-9 are either commercially available or can be prepared according to procedures available from the public domain, as understandable to the person skilled in the art. Specific examples are described in the subsequent paragraphs.

A suitably substituted aromatic ketone of general formula (1-1), such as, for example, 1-(4-nitrophenyl)ethanone, can be condensed with a suitable heteroaryl carbonyl compounds (1-2), such as, for example, quinoline-5-carbaldehyde, in the presence of a base followed by an acid, such as, for example, ammonium acetate followed by formic acid, in a suitable solvent system, such as, for example, THE at temperatures ranging from 0° C. to boiling point of the respective solvent, preferably the reaction is carried out at 0° C., to furnish enones of general formula (1-3).

Enones of general formula (1-3) can be converted to cyano ketones of general formula (1-4) by reaction with a suitably cyanid, such as, for example, acetoncyanhydrin, in the presence of a suitable catalyst, such as, for example (9S)-1-[3,5-bis(trifluoromethyl)benzyl]-6′,9-dimethoxycinchonan-1-ium bromide, in a suitable solvent system, such as, for example, toluene, in a temperature range from −20° C. to the boiling point of the respective solvent, preferably the reaction is carried out between 0° C. and 40° C.

Intermediates of general formula (1-4) can be reacted with a suitable hydrazine, such as, for example, hydrazine monohydrate in the presence of a suitable acid, for example, such as for example acetic acid, in a suitable solvent system, such as, for example ethanol, at temperatures ranging from 0° C. to boiling point of the respective solvent, preferably the reaction is carried out at 100° C., to furnish intermediates of general formula (1-5).

Intermediates of general formula (1-5) can be reduced to intermediates of general formula (1-6). with hydrogen in the presence of an suitable catalyst, for example, palladium on charcoal, in a suitable solvent system, such as, for example ethanol, at temperatures ranging from 0° C. to boiling point of the respective solvent, preferably the reaction is carried out at room temperature, to furnish intermediates of general formula (1-6).

Intermediates of general formula (1-6) are treated with a carbonate of general formula (1-7), such as, for example, 1,1′-[carbonylbis(oxy)]dipyrrolidine-2,5-dione, in the presence of a suitable base, such as for example, N,N-dimethylpyridin-4-amine, in a suitable solvent system, such as, for example, DMF, at a temperature between 0° C. and the boiling point of the respective solvent, preferably the reaction is carried out at room temperature to form the desired intermediate of general formula (1-8).

Intermediates of general formula (1-8) can be converted to compounds of formula (Ia) by reaction with a suitably substituted amine of the general formula (1-11), such as, for example, 2,3-dihydro-1H-pyrrolo[3,4-c]pyridine, in the presence of a suitable base, such as, for example triethylamine, in a suitable solvent system, such as, for example, DMF, in a temperature range from 0° C. to the boiling point of the respective solvent, preferably the reaction is carried out at room temperature.

An alternative route for the preparation of compounds of formula (Ia) is described in Scheme 2.

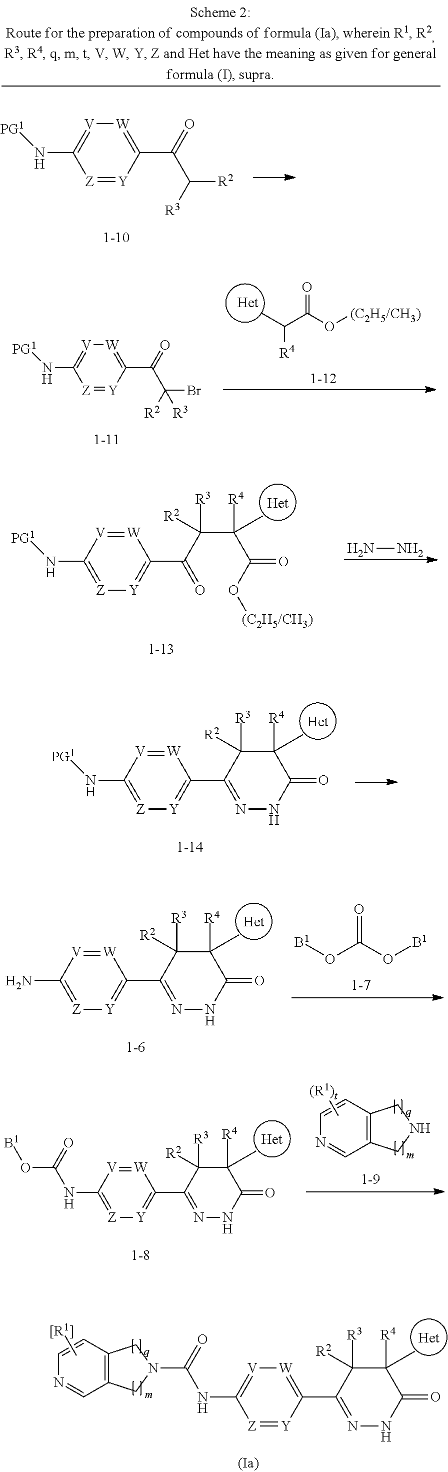

B¹ represents a leaving group such as for example a halo alkyl such for example trichloromethyl or a imid such as, for example pyrrolidine-2,5-dione.

PG¹ represents an amine protecting group, such as, for example, an acetyl group.

In addition, interconversion of any of the substituents R¹, R², R³, R⁴, B¹, m, q, t, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-7, 1-10, and 1-12 are either commercially available or can be prepared according to procedures available from the public domain, as understandable to the person skilled in the art. Specific examples are described in the subsequent paragraphs.

A suitably substituted aromatic ketone of general formula (1-10), such as, for example, tert-butyl (4-acetylphenyl)carbamate, can be brominated with a suitable bromination reagent, such as, for example, N-bromosuccinimide, in a suitable solvent system, such as, for example, THE and water, at temperatures ranging from −20° C. to boiling point of the respective solvent, preferably the reaction is carried out at 0° C., to furnish α-bromo ketones of general formula (1-11).

α-Bromo ketones of general formula (1-11) can be reacted with intermediates of general formula (1-12) such as, for example, ethyl-2-(5-methyl-1,3,4-oxadiazol-2-yl)acetate in the presence of a suitable base, such as, for example, potassium bis(trimethylsilylamide), in a suitable solvent system, such as, for example, THF, in a temperature range from −20° C. to the boiling point of the respective solvent, preferably the reaction is carried out at 0° C., to furnish ketoesters of general formula (1-13)

Ketoesters of general formula (1-13) can be reacted with a suitable hydrazine, such as, for example, hydrazine monohydrate in the presence of a suitable acid, such as, for example acetic acid, in a suitable solvent system, such as, for example dichloromethan, at temperatures ranging from 0° C. to boiling point of the respective solvent, preferably the reaction is carried out at 40° C. to furnish intermediates of general formula (1-14).

Intermediates of general formula (1-14) can be reacted with a suitable Broensted acid, such as, for example, trifluoroacetic acid, in a suitable solvent system, such as, for example, dichloromethane, at temperatures ranging from 0° C. to boiling point of the respective solvent, preferably the reaction is carried out at room temperature, to furnish intermediates of general formula (1-6).

Intermediates of general formula (1-6) are treated with a carbonate of general formula (1-7), such as, for example, 1,1′-[carbonylbis(oxy)]dipyrrolidine-2,5-dione, in the presence of a suitable base, such as for example, N,N-dimethylpyridin-4-amine, in a suitable solvent system, such as, for example, DMF, at a temperature between 0° C. and the boiling point of the respective solvent, preferably the reaction is carried out at room temperature to form the desired intermediate of general formula (1-8).

Intermediates of general formula (1-8) can be converted to compounds of formula (Ia) by reaction with a suitably substituted amine of the general formula (1-9), such as, for example, 2,3-dihydro-1H-pyrrolo[3,4-c]pyridine, in the presence of a suitable base, such as, for example triethylamine, in a suitable solvent system, such as, for example, DMF, in a temperature range from 0° C. to the boiling point of the respective solvent, preferably the reaction is carried out at room temperature.

An alternative route for the preparation of compounds of formula (Ia) is described in Scheme 3.

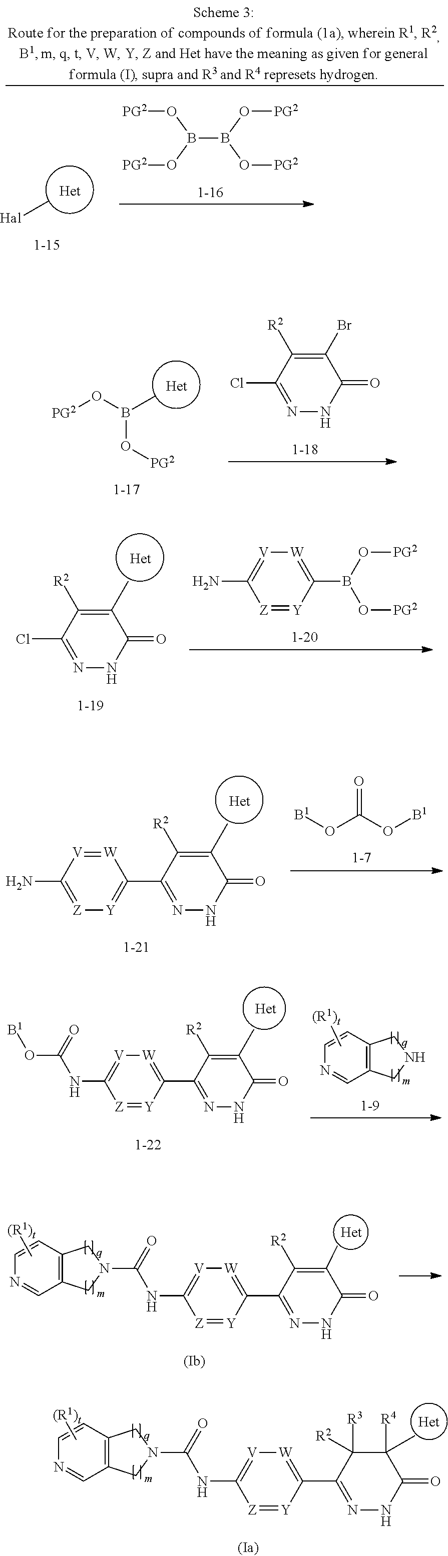

B¹ represents a leaving group such as, for example, a haloalkyl such as, for example, trichloromethyl or a imide such as, for example, pyrrolidine-2,5-dione or p-nitrophenyl.

PG² represents a boronic acid protecting group, such as, for example, an ethyl group or in combination with a second PG².a 2,3 substituted 2,3-dimethylbutan.

In addition, interconversion of any of the substituents R¹, R², R⁴, B¹, m, q, t, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents.

Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-7, 1-9, 1-15, 1-17, and 1-19 are either commercially available or can be prepared according to procedures available from the public domain, as understandable to the person skilled in the art. Specific examples are described in the subsequent paragraphs.

A suitably substituted aromatic halide of general formula (1-15), such as, for example, 7-bromoquinoline, can be reacted with a suitable diboron reagent (1-16), such as, for example, 4,4,4′,4′,5,5,5′,5′-octamethyl-2,2′-bi-1,3,2-dioxaborolane, in the presence of a suitable palladium catalyst, such as, for example, bis(diphenylphosphino)ferrocene]dichloropalladium and a suitable weak base, such as, for example, potassium acetate, in a suitable solvent system, such as, for example, dioxane, at temperatures ranging from 20° C. to boiling point of the respective solvent, preferably the reaction is carried out at 110° C., to furnish intermediates of general formula (1-17).

Intermediates of general formula (1-17) can be coupled with bromo pyridazinones of the general formula (1-18) in the presence of a suitable palladium catalyst, such as, for example, bis(diphenylphosphino)ferrocene]dichloropalladium and a suitable base, such as, for example, potassium carbonate, in a suitable solvent system, such as, for example, dioxane and water, at temperatures ranging from 20° C. to boiling point of the respective solvent, preferably the reaction is carried out at 90° C., to furnish intermediates of general formula (1-19).

Intermediates of general formula (1-19) can be coupled with boronic acid derivates of general formula (1-20) in the presence of a suitable palladium catalyst, such as, for example, bis(diphenylphosphino)ferrocene]dichloropalladium and a suitable base, such as, for example, potassium acetate, in a suitable solvent system, such as, for example, dioxane, at temperatures ranging from 20° C. to boiling point of the respective solvent, preferably the reaction is carried out at 100° C., to furnish intermediates of general formula (1-21).

Intermediates of general formula (1-20) can be converted to intermediates of general formula (1-19) by reaction with a suitable hydrazine of the formula (1-7), such as, for example, hydrazine monohydrate, in a suitable solvent system, such as, for example, propan-1-ol, in a temperature range from −20° C. to the boiling point of the respective solvent, preferably the reaction is carried out at 80° C.

Intermediates of general formula (1-19) can be reacted with a suitable substituted carbamate, such as, for example tert-butyl carbamate (1-20), in the presence of a suitable base, such as, for example caesium carbonate, and a suitable palladium catalyst, such as for example bis(dibenzylideneacetone)-palladium(0), in the presence of a suitable ligand, such as for example 9(9,9-dimethyl-9H-xanthene-4,5-diyl)bis(diphenylphosphine), in a suitable solvent system, such as, for example, 1,4-dioxane, in a temperature range from room temperature to the boiling point of the respective solvent, preferably the reaction is carried out at at 110° C. to furnish compounds of formula (1-21). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phenyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Intermediates of general formula (1-21) can be converted to intermediates of general formula (1-8) by reaction with suitable Broensted acid, such as, for example trifluoroacetic acid, in a suitable solvent system, such as, for example, dichloromethane, in a temperature range from −20° C. to the boiling point of the respective solvent, preferably the reaction is carried out at room temperature.

Intermediates of general formula (1-21) are treated with a carbonate of general formula (1-7), such as, for example, 1,1′-[carbonylbis(oxy)]dipyrrolidine-2,5-dione, in the presence of a suitable base, such as for example, N,N-dimethylpyridin-4-amine, in a suitable solvent system, such as, for example, DMF, at a temperature between 0° C. and the boiling point of the respective solvent, preferably the reaction is carried out at room temperature to form the desired intermediate of general formula (1-22).

Intermediates of general formula (1-22) can be converted to compounds of formula (Ib) by reaction with a suitably substituted amine of the general formula (1-9), such as, for example, 2,3-dihydro-1H-pyrrolo[3,4-c]pyridine, in the presence of a suitable base, such as, for example triethylamine, in a suitable solvent system, such as, for example, DMF, in a temperature range from 0° C. to the boiling point of the respective solvent, preferably the reaction is carried out at room temperature.

Compounds of general formula (Ib) can be converted to compounds of formula (Ia) by reaction with a suitable reducing agent in a suitable solvent, such as zinc in acetic acid, at a temperature between 25° C. and the boiling point of the respective solvent.

A route for the preparation of compounds of formula (1-6) is described in Scheme 4.

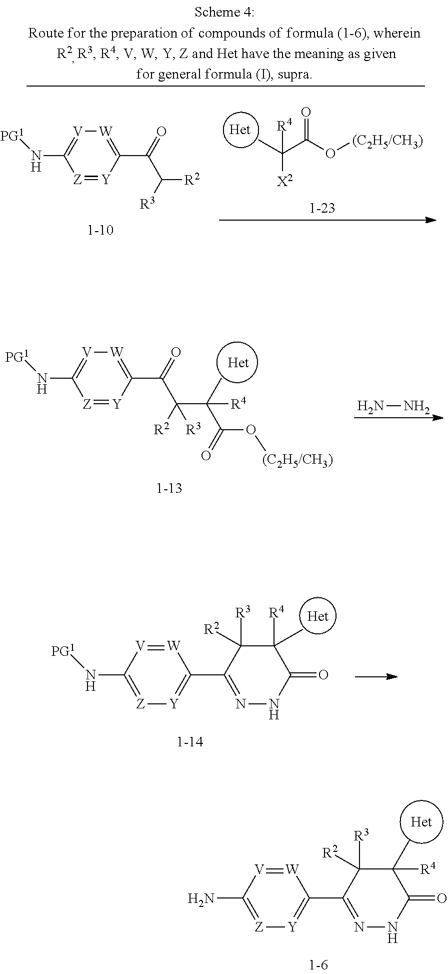

X² represents a leaving group such as for example a Cl or Br atom or an aryl sulfonate such as for example p-toluene sulfonate, or a alkyl sulfonate such as for example methane sulfonate or trifluoromethane sulfonate (triflate group).

PG¹ represents an amine protecting group, such as, for example, an acetyl group.

In addition, interconversion of any of the substituents R², R³, V, W, Y and Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-10, and 1-23 are either commercially available or can be prepared according to procedures available from the public domain, as understandable to the person skilled in the art. Specific examples are described in the subsequent paragraphs.

A suitably substituted aromatic ketone of general formula (1-10), such as, for example, N-(4-propionylphenyl)acetamide, can be reacted with a suitable substituted intermediate of general formula (1-23), such as, for example, ethyl bromoacetate, in the presence of a suitable base, such as, for example, lithium 1,1,1,3,3,3-hexamethyldisilazan-2-ide, in a suitable solvent system, such as, for example, THF, at temperatures ranging from −100° C. to boiling point of the respective solvent, preferably the reaction is carried out at −78° C., to furnish intermediates of general formula (1-24).

Intermediates of general formula (1-24) can be converted to intermediates of general formula (1-14) by reaction with a suitable hydrazine of the formula (1-7), such as, for example, hydrazine monohydrate, in a suitable solvent system, such as, for example, propan-1-ol, in a temperature range from −20° C. to the boiling point of the respective solvent, preferably the reaction is carried out at 0° C.

Intermediates of general formula (1-14) can be reacted with a suitable Broensted acid, such as, for example, hydrochloric acid or sulphuric acid, at temperatures ranging from 0° C. to boiling point of the respective Broensted acid, preferably the reaction is carried out at 100° C., to furnish intermediates of general formula (1-8).

An alternative route for the preparation of compounds of formula (Ia) is described in Scheme 5.

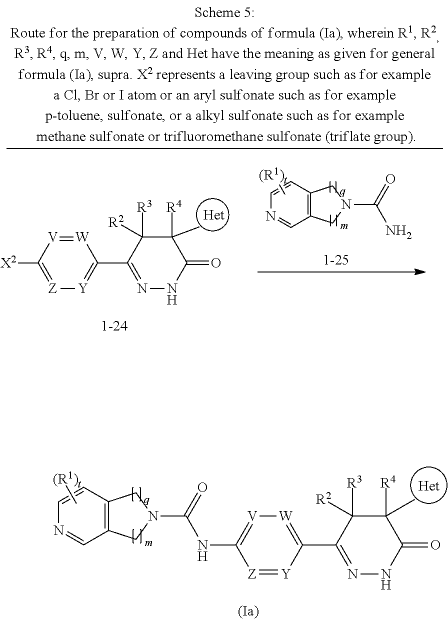

In addition, interconversion of any of the substituents R¹, R², R³, R⁴, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Intermediates of general formula (1-24) can be reacted with a suitable urea of the general formula (1-25), such as, for example 4-(pyridin-3-yl)piperazine-1-carboxamide, in the presence of a suitable base, such as, for example caesium carbonate, and a suitable palladium catalyst, such as for example bis(dibenzylideneacetone)-palladium(0), in the presence of a suitable ligand, such as for example 9(9,9-dimethyl-9H-xanthene-4,5-diyl)bis(diphenylphosphine), in a suitable solvent system, such as, for example, 1,4-dioxane, in a temperature range from room temperature to the boiling point of the respective solvent, preferably the reaction is carried out at at 110° C. to furnish compounds of formula (Ia). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phenyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

An alternative route for the preparation of compounds of formula (Ia) is described in Scheme 6.

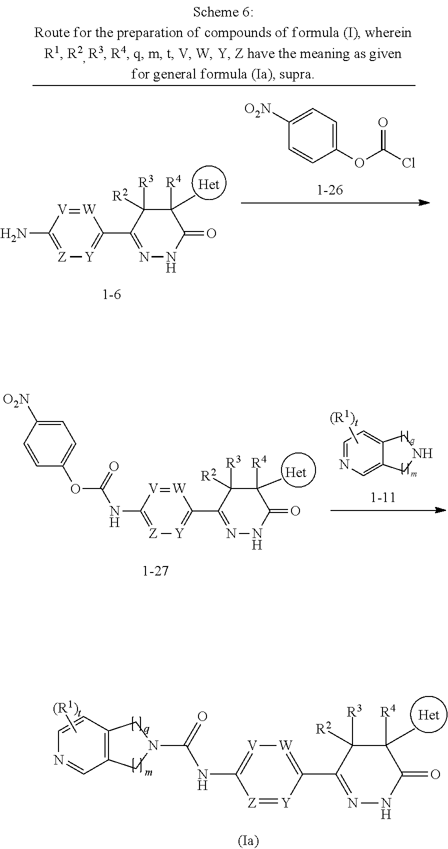

In addition, interconversion of any of the substituents R¹, R², R³, R⁴, m, n, t, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-11 and 1-26 are either commercially available or can be prepared according to procedures available from the public domain, as understandable to the person skilled in the art. Specific examples are described in the subsequent paragraphs.

Intermediates of general formula (1-6) can be converted to intermediates of general formula (1-27) by reaction with a suitably chloroformate of the general formula (1-26), such as, for example, 4-nitrophenyl carbonochloridate, in the presence of a suitable base, such as, for example, triethylamine, in a suitable solvent system, such as, for example, toluene, in a temperature range from −20° C. to the boiling point of the respective solvent, preferably the reaction is carried out at room temperature.

Intermediates of general formula (1-27) can be converted to compounds of formula (Ia) by reaction with a suitably amine of the general formula (1-11), such as, for example, 1-(pyridin-3-yl)piperazine, in a suitable solvent system, such as, for example, ethanol, in a temperature range from −20° C. to the boiling point of the respective solvent, preferably the reaction is carried out at 79° C.

One route for the preparation of compounds of formula (1-10) is described in Scheme 7.

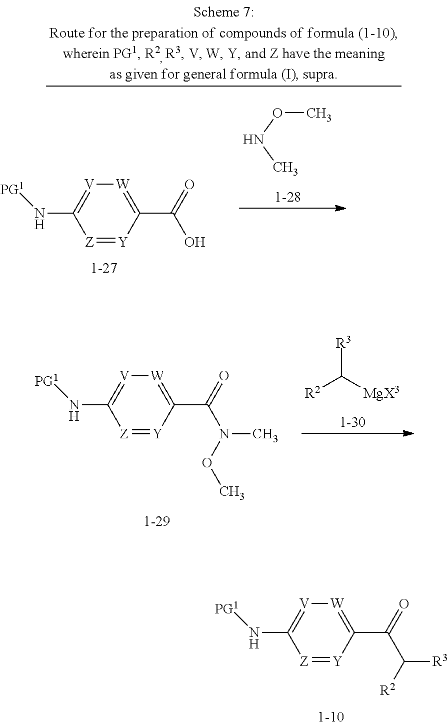

X³ represents a halogen atom such as for example a Cl or Br atom.

PG¹ represents an amine protecting group as for example a fluorenylmethyloxycarbonyl, benzyloxycarbonyl, allyloxycarbonyl or tert-butyloxycarbonyl group.

In addition, interconversion of any of the substituents PG¹, R², R³, V, W, Y and Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-27, 1-28 and 1-30 are either commercially available or can be prepared according to procedures available from the public domain, as understandable to the person skilled in the art. Specific examples are described in the subsequent paragraphs.

Compounds of formula (1-27) are reacted with a compound of formula (1-28) as mentioned above with a peptide coupling agent, for example N-(3-Dimethylaminopropyl)-N′-ethylcarbodiimide hydrochloride, in the presence of 1-hydroxy-7-azabenzotriazole in a suitable solvent, such as, for example, N,N-dimethylformamid, in the presence of a suitable base, such as, for example, triethylamine in a temperature range from −10° C. to the boiling point of the respective solvent, preferably the reaction is carried out at room temperature, to furnish compounds of formula (1-29).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

Intermediates of general formula (1-29) can be converted to compounds of general formula (1-10) by reaction with a suitably Grignard reagent of the general formula (1-30), such as, for example, benzylmagnesium chloride, in a suitable solvent system, such as, for example, tetrahydrofurane, in a temperature range from −20° C. to the boiling point of the respective solvent, preferably the reaction is carried out at 0° C.

It is known to the person skilled in the art that, if there are a number of reactive centers on a starting or intermediate compound, it may be necessary to block one or more reactive centers temporarily by protective groups in order to allow a reaction to proceed specifically at the desired reaction center. A detailed description for the use of a large number of proven protective groups is found, for example, in T. W. Greene, Protective Groups in Organic Synthesis, John Wiley & Sons, 1999, 3rd Ed., or in P. Kocienski, Protecting Groups, Thieme Medical Publishers, 2000.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present invention which possess a sufficiently basic or acidic functionality, may result in the formation of a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, solvate, inclusion complex) of a compound of the present invention as isolated as described herein is not necessarily the only form in which said compound can be applied to a biological assay in order to quantify the specific biological activity.

Salts of the compounds of formula (Ia), or (Ib) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the examples section.

Pure diastereomers and pure enantiomers of the compounds and salts according to the invention can be obtained e.g. by asymmetric synthesis, by using chiral starting compounds in synthesis and by splitting up enantiomeric and diasteriomeric mixtures obtained in synthesis.

Enantiomeric and diastereomeric mixtures can be split up into the pure enantiomers and pure diastereomers by methods known to a person skilled in the art. Preferably, diastereomeric mixtures are separated by crystallization, in particular fractional crystallization, or chromatography. Enantiomeric mixtures can be separated e.g. by forming diastereomers with a chiral auxiliary agent, resolving the diastereomers obtained and removing the chiral auxiliary agent. As chiral auxiliary agents, for example, chiral acids can be used to separate enantiomeric bases such as e.g. mandelic acid and chiral bases can be used to separate enantiomeric acids via formation of diastereomeric salts. Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxiliary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

Optionally, compounds of the formula (Ia), or (Ib) can be converted into their salts, or, optionally, salts of the compounds of the formula (Ia) or (Ib) can be converted into the free compounds. Corresponding processes are customary for the skilled person.

Optionally, compounds of the formula (Ia), or (Ib) can be converted into their N-oxides. The N-oxide may also be introduced by way of an intermediate. N-oxides may be prepared by treating an appropriate precursor with an oxidizing agent, such as meta-chloroperbenzoic acid, in an appropriate solvent, such as DCM, at suitable temperatures, such as from 0° C. to 40° C., whereby room temperature is generally preferred. Further corresponding processes for forming N-oxides are customary for the skilled person.

One preferred aspect of the invention is the process for the preparation of the conjugates and compounds according to the examples, as well as the intermediates used for their preparation.

The NAMPT inhibitors of formula (II-D), and (III-D):

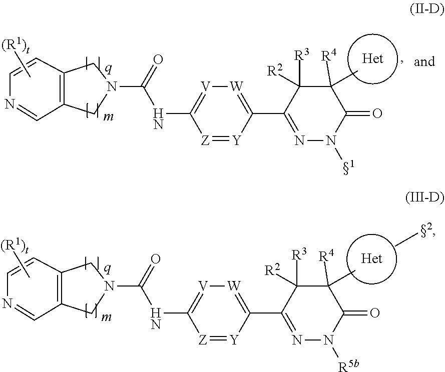

wherein §¹ and §² represents the point of attachment to linker Z′ and R¹, R², R³, R⁴, R^{5b}, q, m, t, V, W, Y, Z and Het are as defined herein, can be made according to any of the methods described in the present application, or alternatively, according to any of the methods described in WO2012067965 (each of the methods of WO2012067965 being incorporated herein by reference)

A method to synthesize compounds according to structure (IIa) is alkylation of compounds I, meaning compounds of structure (Ia) or (Ib), at the pyridazinone NH with a compound of type 1-31 as depicted in Scheme 8.

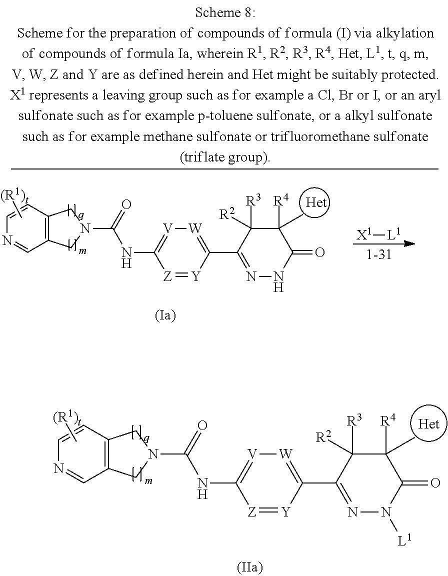

Conditions for this alkylation can be typical conditions known to those skilled in the art like treatment of Ia with a suitable base, such as, for example sodium hydride, in a suitable solvent system, such as, for example, DMF, followed by addition of an alkylating compound of structure 1-31 in a temperature range from −20° C. to the boiling point of the respective solvent, preferably the reaction is carried out between 0° C. and r.t.

A method to synthesize compounds according to structure (IIb) is alkylation of compounds I, meaning compounds of structure (Ic), where a NH group is present at Het with a compound of type 1-31 as depicted in Scheme 9.

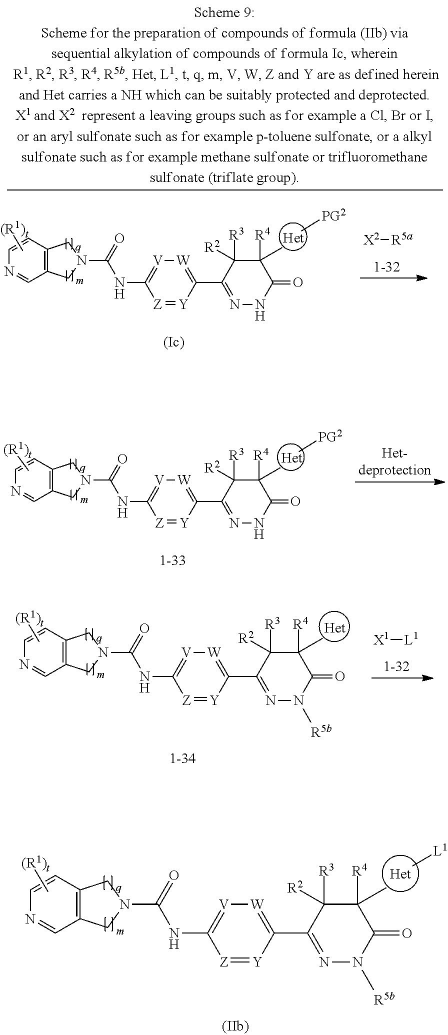

Conditions for the first alkylation can be typical conditions known to those skilled in the art like treatment of Ia with a suitable base, such as, for example sodium hydride, in a suitable solvent system, such as, for example, DMF, followed by addition of an alkylating compound of structure 1-32 in a temperature range from −20° C. to the boiling point of the respective solvent, preferably the reaction is carried out between 0° C. and r.t.

PG² represents an heterocyclyl-NH protecting group as for example a tetrahydropyranyl group protecting an indazol NH, a p-toluoyl sulfonyl group protecting a benzimidazol-NH or a 2-(trimethylsilyl)ethoxycarbonyl group protecting an indol-NH.

In addition, interconversion of any of the substituents PG², R², R³, V, W, Y and Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art.

Compounds of formula (1-33) can be deprotected as mentioned above with a deprotection agent, for example hydrochlorid acid in the case of a tetrahydropyranyl protected indazole in a suitable solvent, such as, for example, a mixture of cyclopentyl methyl ether and water, in a temperature range from −10° C. to the boiling point of the respective solvent, preferably the reaction is carried out at room temperature, to furnish compounds of formula (1-34).

Conditions for the second alkylation at the heterocyclyl-NH of compound (1-34) can be typical conditions known to those skilled in the art like treatment of Ia with a suitable base, such as, for example sodium hydride, in a suitable solvent system, such as, for example, DMF, followed by addition of an alkylating compound of structure 1-31 in a temperature range from −20° C. to the boiling point of the respective solvent, preferably the reaction is carried out between 0° C. and r.t.

Compounds of type of type 1-37 which are compounds of general structure (II) wherein L¹ bears an amino substituent can be prepared by a route as described in scheme 10. Pyridazinone of general structure (Ia) is reacted with an alkylating agent of structure 1-35 wherein R¹³ is a protected amine group like NH-PG¹, phthalimide, nitro or azide under conditions known to those skilled in the art to give N-alkylated compound of structure 1-36. These methods include treatment of (Ia) with a suitable base, such as, for example sodium hydride, in a suitable solvent system, such as, for example, DMF, followed by addition of an alkylating compound of structure 1-35 in a temperature range from −20° C. to the boiling point of the respective solvent, preferably the reaction is carried out between 0° C. and r.t. The resulting compound of structure 1-36 is then converted into amine of structure 1-37 by transforming N-protected moiety R¹³ to NH₂ by methods known to those skilled in the art, like acidic cleavage of the Boc group with TFA or hydrochloric acid, cleavage of the phthalimide group with hydrazine or methyl amine, reduction of the nitro group with iron/acetic acid or hydrogenation with palladium on charcoal under a hydrogen atmosphere or reduction of the azide group by hydrogenation with palladium on charcoal under a hydrogen atmosphere or by Staudinger-type reduction with triphenylphosphine to give compound of structure 1-37.

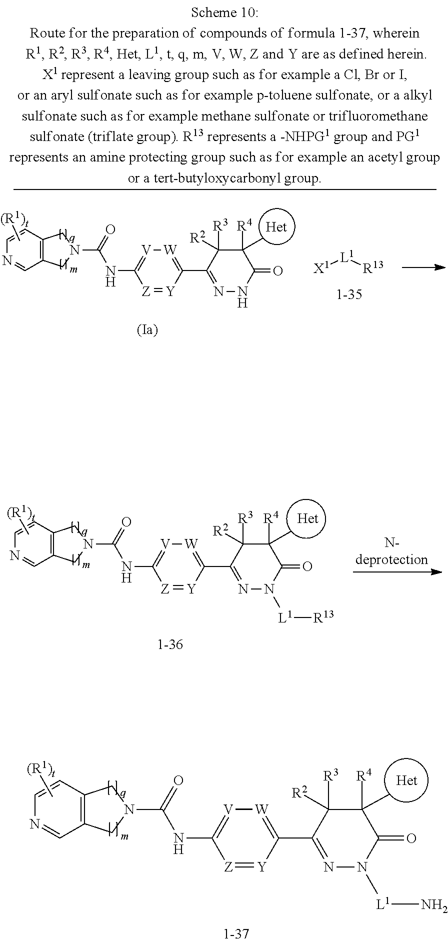

Compounds of structure 1-42 with polyethyleneglycol (PEG) side chains in the linker which are a subclass of general structure 1-37 can be synthesized as described in Scheme 10.

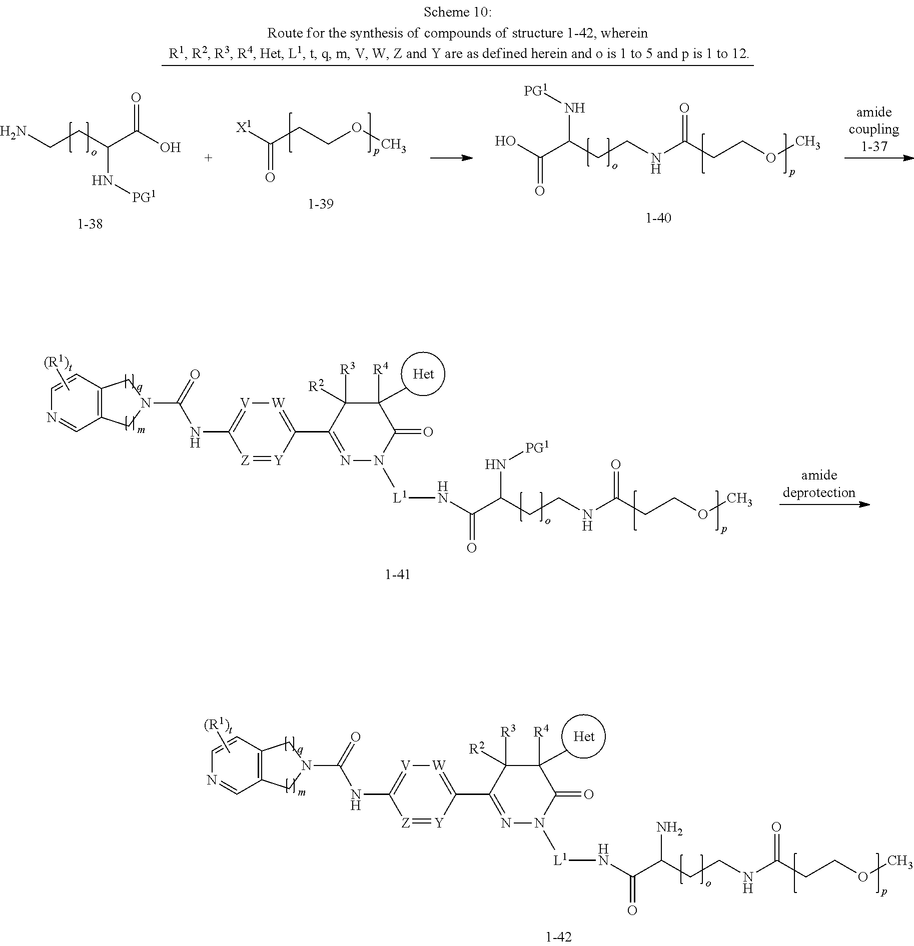

An α-amine protected amino acid of structure 1-38 is coupled to a polyethyleneglycolic active ester of structure 1-39 by amide formation methods known to those skilled in the art. The so formed free acid of structure 1-40 can then be coupled to amine 1-37 to give the corresponding PEGylated amide of structure 1-41. Deprotection of the amine group (methods see above) then give the corresponding amine of structure 1-42.

Compounds of structure 1-48 with peptidic moieties in the linker which are a subclass of general structure 1-37 can be synthesized as described in Scheme 11.

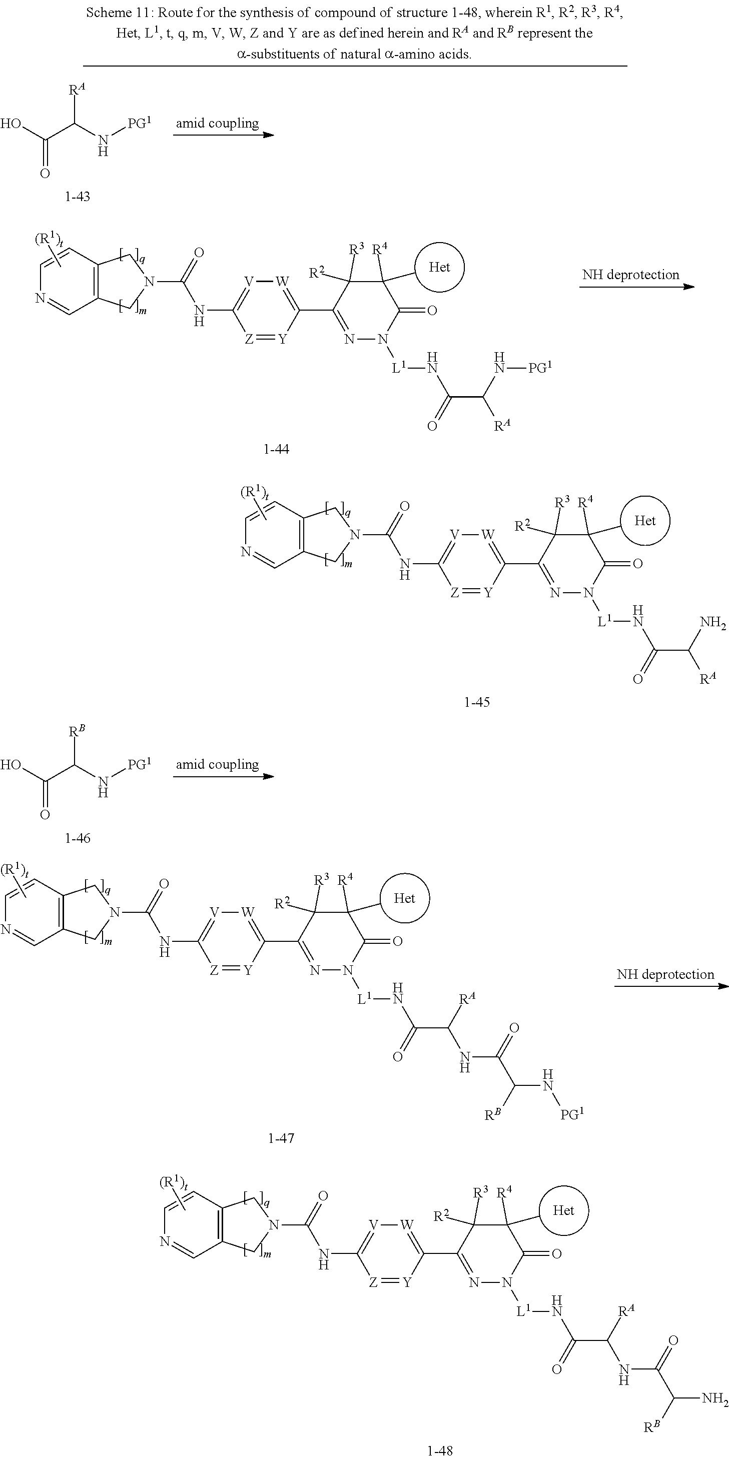

Amine 1-37 can be coupled with an α-amino protected amino acid of structure 1-43 by peptide coupling methods known to those skilled in the art, like e.g. HATU. For further peptide coupling methods see above. The so formed peptide of structure 1-44 can then be deprotected at the amine group my methods known to those skilled in the art (see above) to give compound 1-45. The above described sequence can then be repeated with a second α-amino protected amino acid of structure 1-46 to give the amine of the general structure 1-48 after deprotection by methods known to those skilled in the art.

Compounds of type of type 1-50 which are compounds of general structure (II) wherein L1 bears an amino substituent can be prepared by a route as described in scheme 12. A heterocyclyl-NH compound of general structure 1-34 is reacted with an alkylating agent of structure 1-35 wherein R¹³ is a protected amine group like NH-PG¹, phthalimide, nitro or azide under conditions known to those skilled in the art to give N-alkylated compound of structure 1-49. These methods include treatment of 1-34 with a suitable base, such as, for example sodium hydride, in a suitable solvent system, such as, for example, DMF, followed by addition of an alkylating compound of structure 1-35 in a temperature range from −20° C. to the boiling point of the respective solvent, preferably the reaction is carried out between 0° C. and r.t. The resulting compound of structure 1-49 is then converted into amine of structure 1-50 by transforming N-protected moiety R¹³ to NH₂ by methods known to those skilled in the art, like acidic cleavage of the Boc group with TFA or hydrochloric acid, cleavage of the phthalimide group with hydrazine or methyl amine, reduction of the nitro group with iron/acetic acid or hydrogenation with palladium on charcoal under a hydrogen atmosphere or reduction of the azide group by hydrogenation with palladium on charcoal under a hydrogen atmosphere or by Staudinger-type reduction with triphenylphosphine to give compound of structure 1-50.

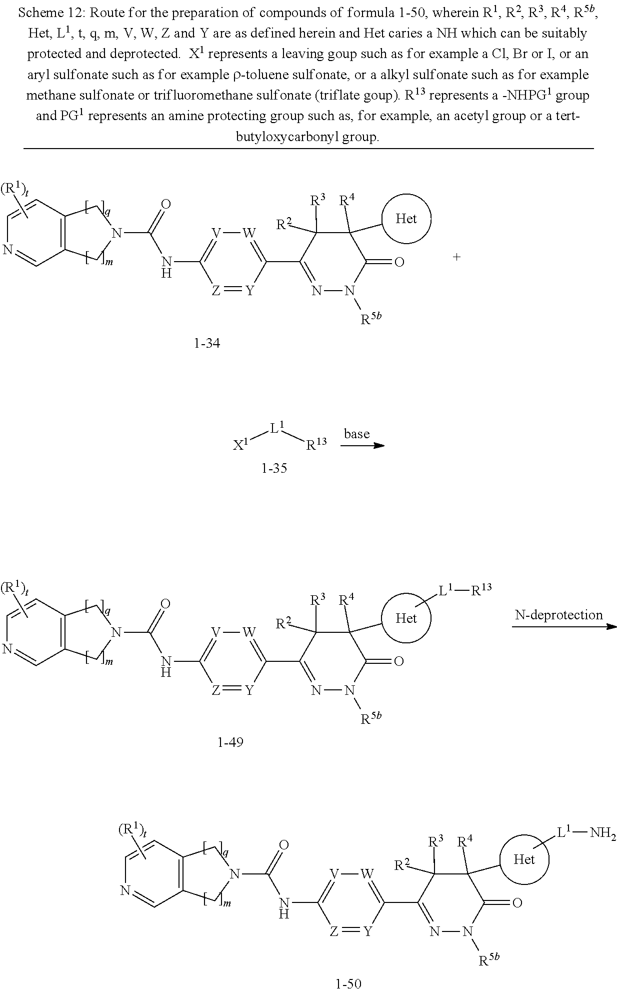

Antibody-drug conjugates of general structure

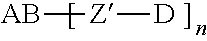

can be synthesized by reacting an activated moiety Z″-D with an antibody AB wherein linker Z″ represents one of the following general structures (i) to (iii):
(i) §¹-L1-SG-L2′-§§ or §²-L1-SG-L2′-§§
(ii) §¹-L1-SG-L1′-L2′-§§ or §²-L1-SG-L1′-L2′-§§
(iii) §¹-L1-L2′-§§ or §²_L1-L2′-§§
   wherein
§¹ and §² represent the attachment point to D;
§§ represents the attachment point to AB;
   SG represents an in vivo cleavable group, L1 and L1′ represent, independently of each other, an in vivo non-cleavable organic group, and L2′ represents an activated attachment group.

For the synthesis of cysteine-linked antibody-drug conjugates L2′ is a thiol-reactive group from the group of

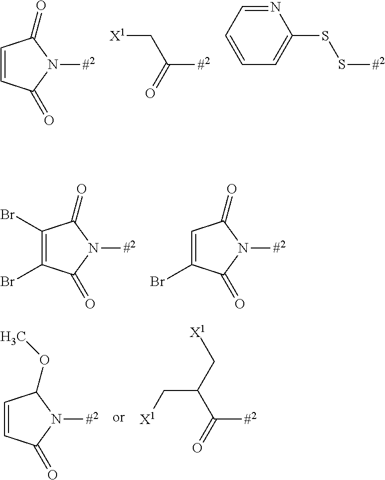

wherein #² represents the attachment point to the group L1, L1′ or SG and X¹ represents a leaving group such as for example a C₁, Br or I, or an aryl sulfonate such as for example p-toluene sulfonate, or a alkyl sulfonate such as for example methane sulfonate or trifluoromethane sulfonate (triflate group).

More preferably, activated moiety Z″-D for the synthesis of cysteine-linked antibody-drug conjugates bears a maleimide as in general structures 1-51 and 1-52

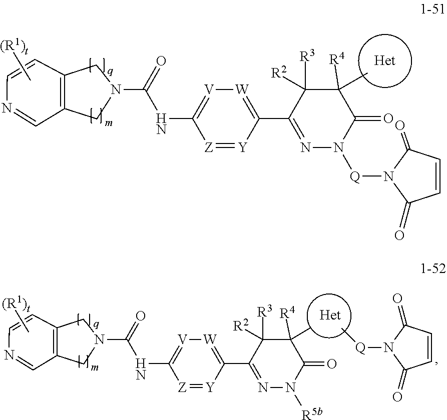

wherein R¹, R², R³, R⁴, R^{5b}, Het, t, q, m, V, W, Z and Y are as defined herein and Q represents one of the following general structures (i) to (iii):
(i) §-L1-SG-§§
(ii) §-L1-SG-L1′-§§
(iii) §-L1-§§
   wherein
§ represents the attachment point to D;
§§ represents the attachment point to L2′;
   SG represents an in vivo cleavable group, L1 and L1′ represent, independently of each other, an in vivo non-cleavable organic group, and L2′ represents an activated attachment group.

Maleimides of general structure 1-54 which are a subclass of general structure 1-51 can be synthesized by reacting amine of general structure 1-37 with activated ester of structure 1-54 as shown in scheme 13.

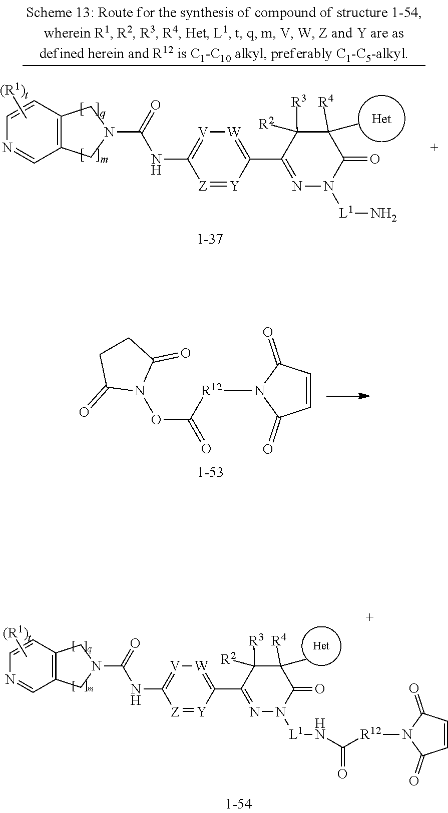

Amine of general structure 1-37 is reacted with an N-hydroxysuccinimidyl ester of structure 1-53 by methods known to those skilled in the art to give maleimide of general structure 1-54. These methods include addition of a base like e.g. triethyl amine or diisoproypethylamine and a suitable solvent like DMF or THF. Alternative methods include coupling of amine 1-37 with the corresponding free acid of structure 1-133 under peptide coupling conditions known to those skilled in the art as described above.

Maleimides of general structure 1-55 which are a subclass of general structure 1-52 can be synthesized by reacting amine of general structure 1-37 with activated ester of structure 1-54 as shown in scheme 14.

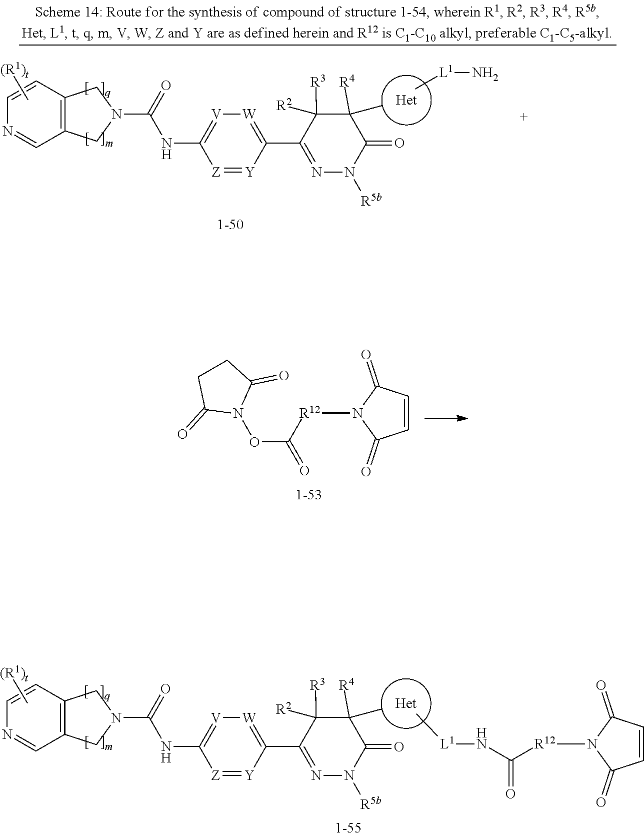

Amine of general structure 1-50 is reacted with an N-hydroxysuccinimidyl ester of structure 1-53 by methods known to those skilled in the art to give maleimide of general structure 1-55. These methods include addition of a base like e.g. triethyl amine or diisoproypethylamine and a suitable solvent like DMF or THF. Alternative methods include coupling of amine 1-37 with the corresponding free acid of structure 1-50 under peptide coupling conditions known to those skilled in the art as described above.

For the synthesis of lysine-linked antibody-drug conjugates L2′ is an amine-reactive group from the group of

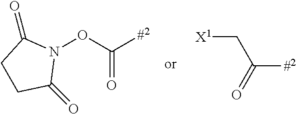

wherein #² represents the attachment point to the group L1, L1′ or SG X¹ represents a leaving group such as for example a C₁, Br or I, or an aryl sulfonate such as for example p-toluene sulfonate, or a alkyl sulfonate such as for example methane sulfonate or trifluoromethane sulfonate (triflate group).

More preferably, activated moiety Z″-D for the synthesis of lysine-linked antibody-drug conjugates bears a N-hydroxysuccinimidyl ester as in general structure 1-56 and 1-57

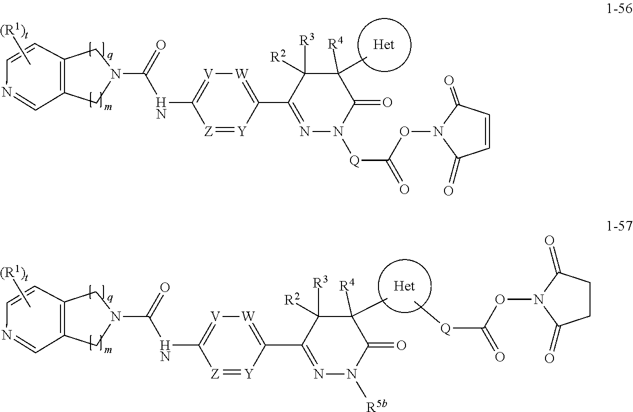

wherein R¹, R², R³, R⁴, R^{5b}, Het, t, q, m, V, W, Z and Y are as defined herein and Q represents one of the following general structures (i) to (iii):
(i) §-L1-SG-§§
(ii) §-L1-SG-L1′-§§
(iii) §-L1-§§
   wherein
§ represents the attachment point to D (pyridazinone ring in 1-56 and rig Het in 1-57);
§§ represents the attachment point to L2′ (at its carbonyl group);
   SG represents an in vivo cleavable group, L1 and L1′ represent, independently of each other, an in vivo non-cleavable organic group, and L2′ represents an activated attachment group.

A method to synthesize N-hydroxysuccinimidyl esters of general structure 1-59 which are a substructure of general structure 1-56 is shown in scheme 15.

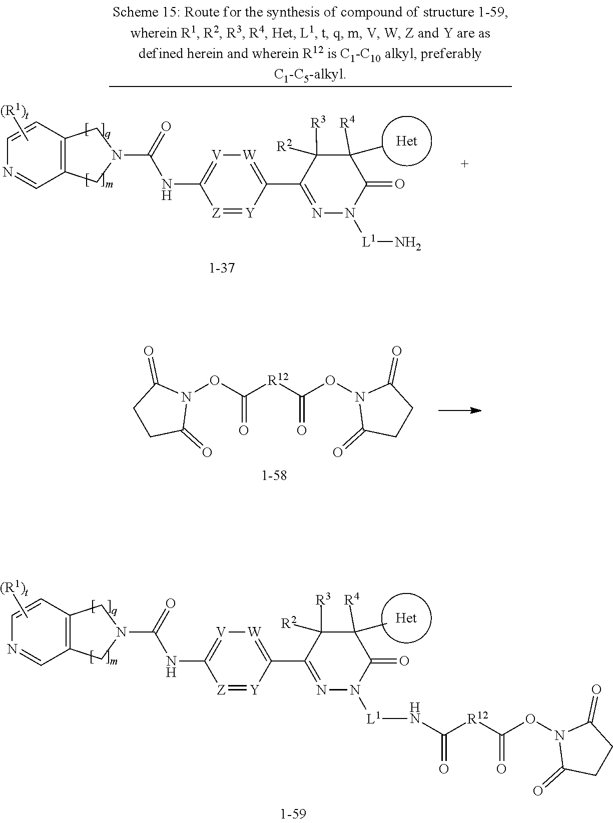

Amine of general structure 1-37 is reacted with a bis-N-hydroxysuccinimidyl ester of structure 1-58 by methods known to those skilled in the art to give maleimide of general structure 1-59. These methods include addition of a base like e.g. triethyl amine or diisoproypethylamine and a suitable solvent like DMF or THF. Alternative methods include coupling of amine 1-37 with the corresponding free acid of structure 1-58 under peptide coupling conditions known to those skilled in the art as described above.

A method to synthesize N-hydroxysuccinimidyl esters of general structure 1-60 which are a substructure of general structure 1-57 is shown in scheme 16.

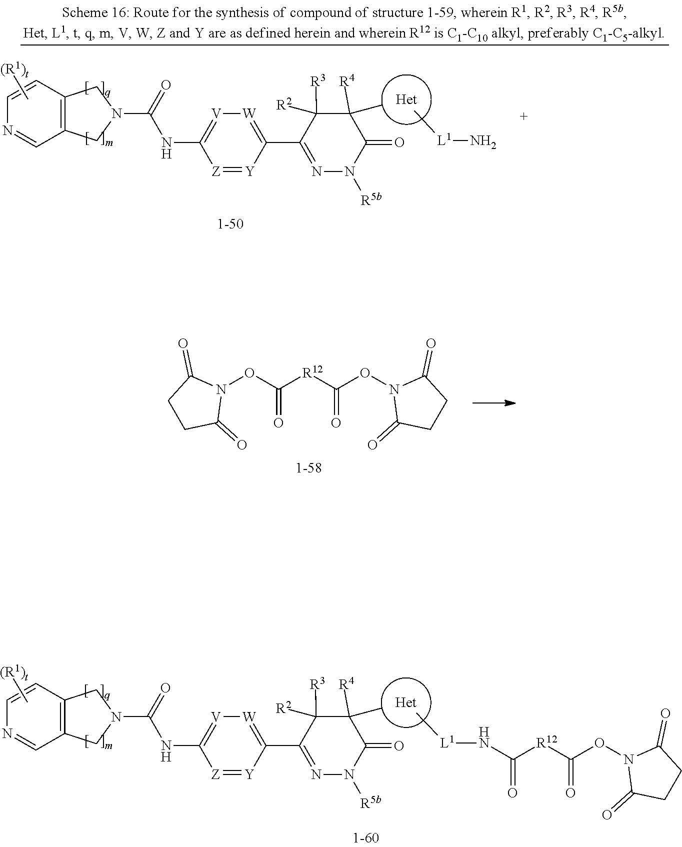

Amine of general structure 1-50 is reacted with a bis-N-hydroxysuccinimidyl ester of structure 1-58 by methods known to those skilled in the art to give maleimide of general structure 1-60. These methods include addition of a base like e.g. triethyl amine or diisoproypethylamine and a suitable solvent like DMF or THF. Alternative methods include coupling of amine 1-50 with the corresponding free acid of structure 1-58 under peptide coupling conditions known to those skilled in the art as described above.

The metabolites of the exemplified antibody-drug conjugates bearing non-cleavable linkers can be described by the general structure 1-61 for cysteine-linked ADCs and 1-60 for lysine-linked conjugates.

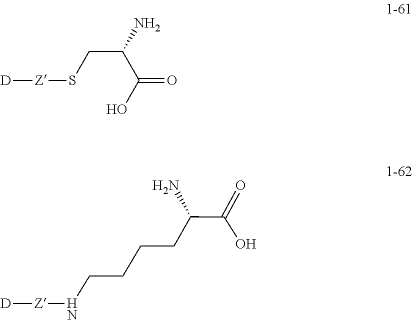

wherein Z′, and D are as defined herein.

More specifically, cysteine-linked antibody-drug conjugates with non-cleavable linkers connected via a succinimide deliver metabolites of general structure 1-64 or 1-65. The metabolites of general structure 1-64 and 1-65 can be synthesized by reacting maleimide of general structure 1—with cysteine 1-63 in a suitable solvent like DMF at a temperature range of 0−40° C., preferably at 25° C. to give cysteine-metabolite 1-64 or 1-65 as described in scheme 17.

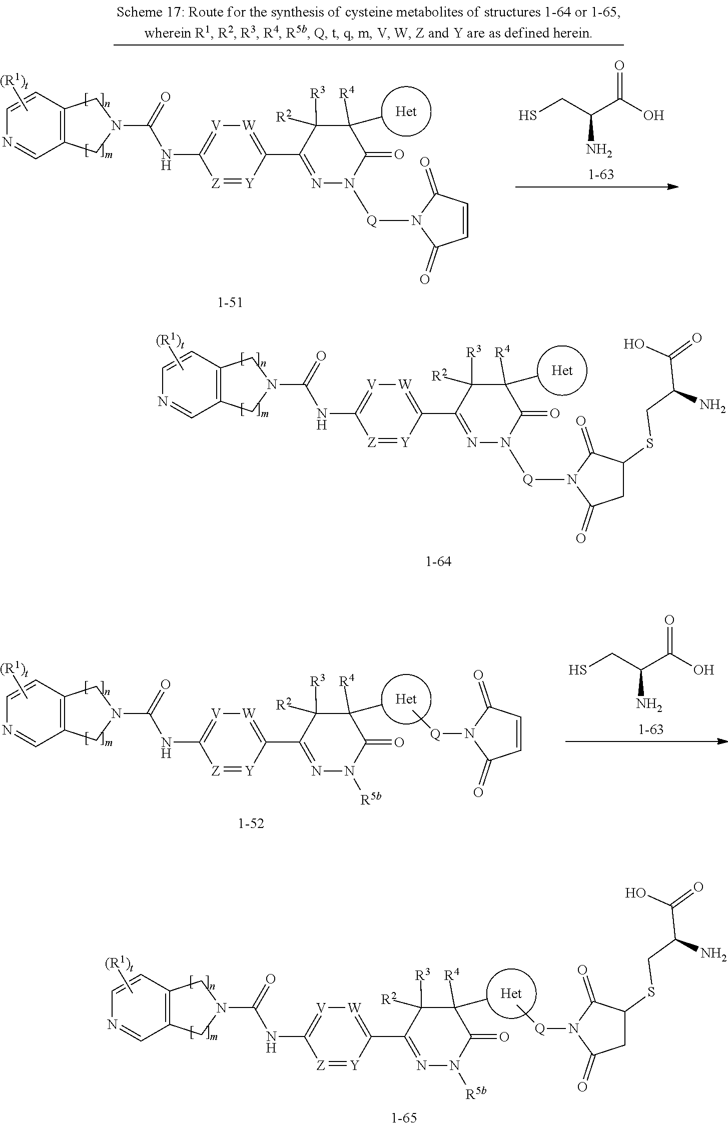

The succinimide ring can also be present in an open form described by general structures 1-64a or 1-65a as shown in scheme 18.

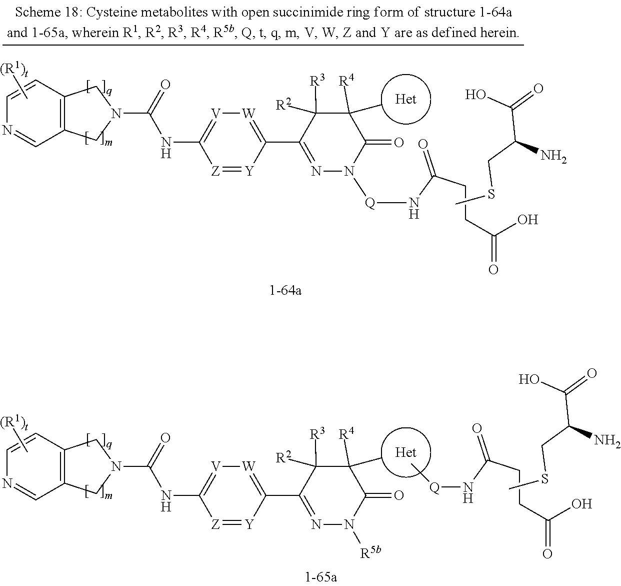

Lysine-linked antibody-drug conjugates with non-cleavable linkers connected via an amide bond deliver metabolites of general structure 1-68. The metabolites of general structure 1-68 can be synthesized by reacting N-hydroxysuccinimidyl esters of general structure 1-56 with α-amino,α-carboxyl-bisprotected lysine (1-66) in a suitable solvent like DMF or THE and a suitable base like triethylamine or diisopropylethylamine at a temperature range of 0-40° C., preferably at 25° C. to give α-amino,α-carboxyl-bisprotected lysine-metabolite of general structure 1-67 as described in scheme 42. Deprotection at the α-amino and α-carboxyl group of lysine conjugate 1-67 by methods known to those skilled in the art as already described above delivers the lysine metabolite of general structure 1-68. An alternative method for the synthesis of 1-67 includes coupling of α-amino,α-carboxyl-bisprotected lysine (1-66) with the corresponding free acid of structure 1-56 under peptide coupling conditions known to those skilled in the art as described above.

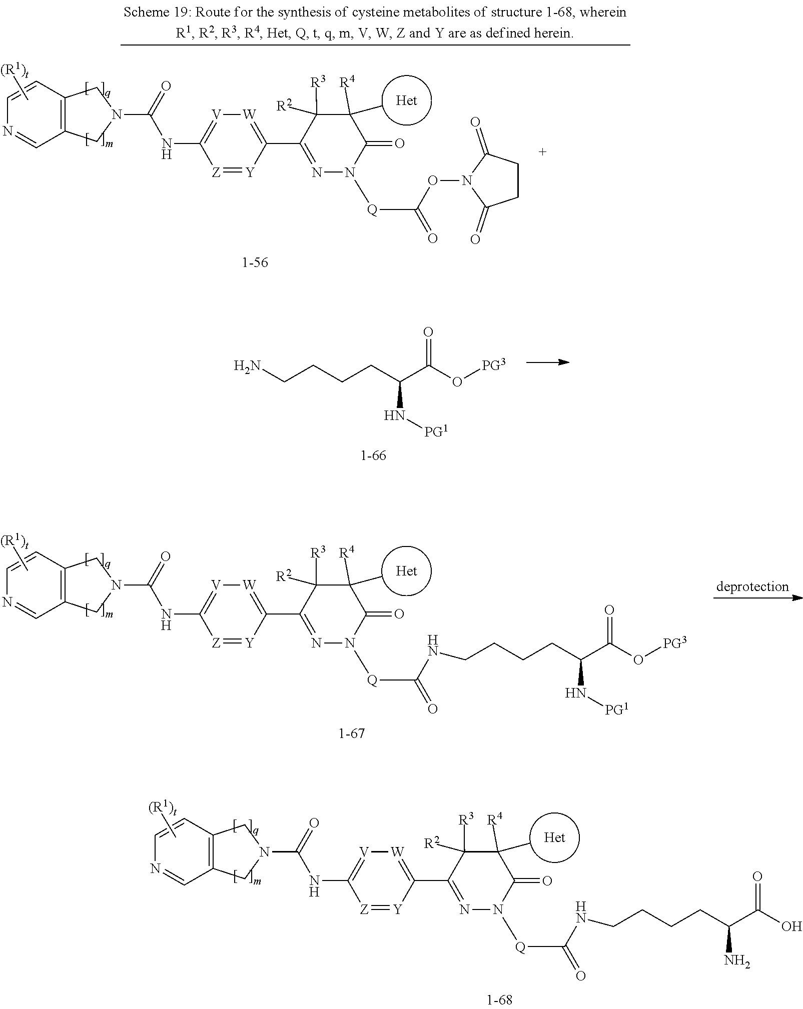

Antibody-drug conjugates of general structure

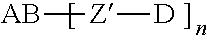

can be synthesized by reacting activated moiety of general structure Z″-D with an antibody.

More specifically cysteine-linked antibody-drug conjugates can be synthesized according to scheme 20.

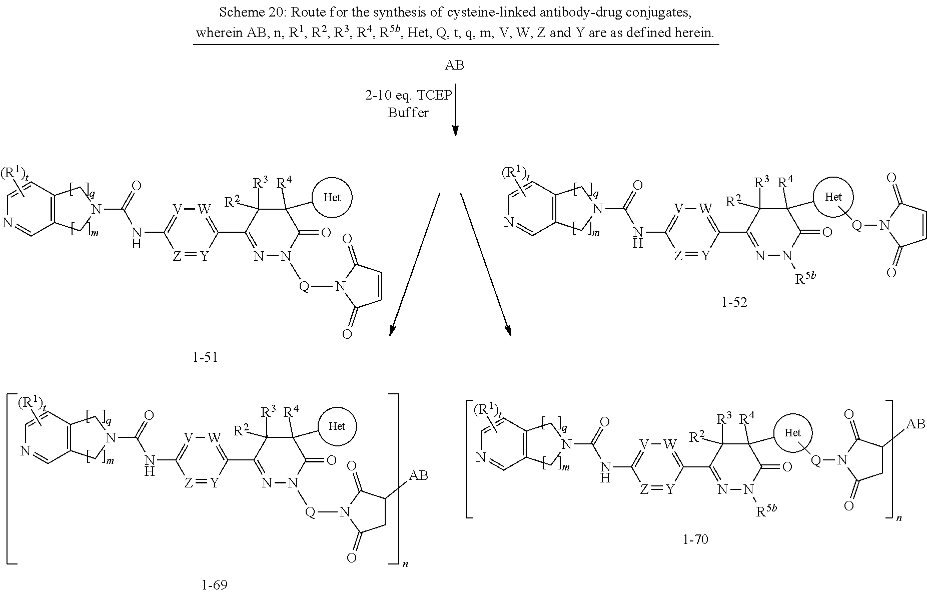

Antibody AB is reduced with triscarboxyethylphosphine (TCEP) to reduce the intrachain disulfides of the antibody in a suitable solvent like PBS buffer at pH 7.2 at a concentration between range between 1 mg/ml and 20 mg/ml, preferably in the range of about 10 mg/ml to 15 mg/ml, at a temperature between 4° C. and 30° C., preferably at 20° C. Then maleimide 1-51 or 1-52 is added dissolved in a suitable solvent like DMSO, DMF, isopropanol or PBS buffer, preferably in DMSO whereas the amount of solvent should not exceed 10% of the total volume.

For the synthesis of cysteine-linked antibody-drug conjugates with a corresponding ring-opened succinimide the reaction mixture can be basified to pH 8 by addition of a suitable base or buffer like PBS buffer pH 8.

In case Q bears a reducible moiety like a disulfide the TCEP is removed after the reduction step prior to the addition of the maleimides 1-51 or 1-52 by a suitable method like size-exclusion chromatography via a Sephadex® column.

Lysine-linked antibody-drug conjugates can be synthesized according to scheme 21.

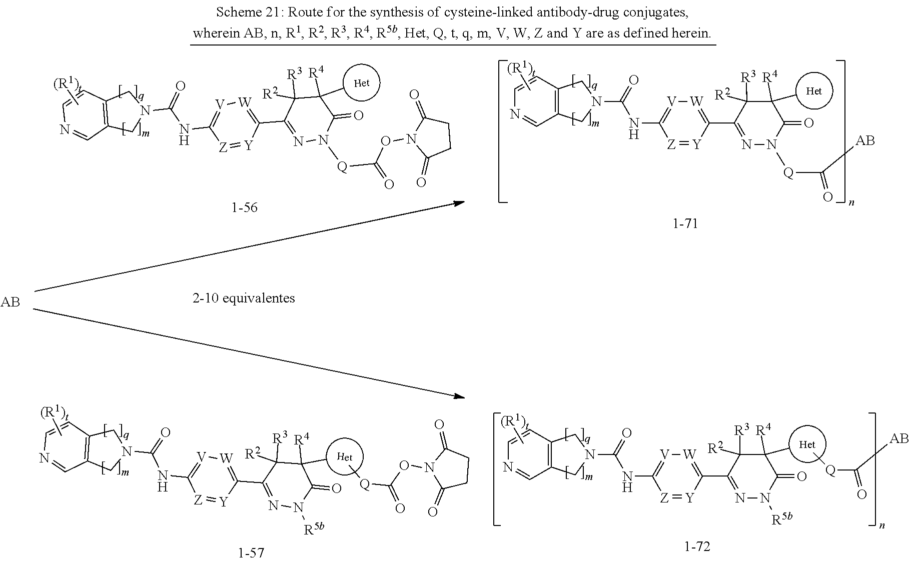

Antibody AB is reacted with N-hydroxysuccinimidyl ester of general structure 1-56 or 1-57, dissolved in a suitable solvent like DMSO, DMF, isopropanol or PBS buffer, preferably in DMSO whereas the amount of solvent should not exceed 10% of the total volume, in a suitable solvent like PBS buffer at pH 7.2 at a concentration between range between 1 mg/ml and 20 mg/ml, preferably in the range of about 10 mg/ml to 15 mg/ml, at a temperature between 4° C. and 30° C., preferably at 20° C.

### Site Specific Conjugation

Site specific conjugation, in which a known number of linker-drugs are consistently conjugated to defined sites might be used.

There are various methods described in literature for site specific conjugation (Agarwal et al., Bioconjug. Chem. 26, 176-192 (2015); Cal et al., Angew. Chem. Int. Ed. Engl. 53, 10585-10587 (2014); Behrens et al., MAbs 6, 46-53 (2014); Panowski et al., MAbs 6, 34-45 (2014)). Methods for site specific conjugation include, in particular, enzymatic methods, e.g using transglutaminases (TGases), glycyltransferases or formylglycine generating enzyme (Sochaj et al., Biotechnology Advances, 33, 775-784 2015).

One way for this attachment using transglutaminases (TGases), are literature-described approaches dealing with a site specific conjugation of binders using transglutaminase.

Transglutaminases (TGase) including bacterial transglutaminase (BTG) (EC 2.3.2.13) are a family of enzymes that catalyze the formation of a covalent bond between the γ-carbonyl amide group of glutamines and the primary amine of lysines. Since some TGases also accept substrates other than lysine as the amine donor, they have been used to modify proteins including antibodies at suitable acceptor glutamine residues (Jeger et al., Angewandte Chemie Int. Ed. Engl 49, 9995-9997 (2010); Josten et al., J. Immunol. Methods 240, 47-54 (2000); Mindt et al., Bioconjugate Chem. 19, 271-278 (2008); Dennler et al., in Antibody Drug Conjugates (Ducry, L., Ed.), pp 205-215, Humana Press. (2013)). On the one hand transglutaminases were used for coupling of drugs to antibodies bearing genetically artificial glutamine tags being transglutaminse acceptor glutamines introduced by genetically engineering (Strop et al., Chem. Biol. 20, 161-167 (2013)). On the other hand it was reported that the conserved glutamine Q295 (Kabat numbering system of IgGs) located in the constant domain of the heavy chain is the sole γ-carbonyl amide donor for bacterial transglutaminase (EC 2.3.2.13) within the backbone of a aglycosylated IgG1, whereas no acceptor glutamine is present in the backbone in IgG1 being glycosylated at position N297 (kabat numbering) of the heavy chain (Jeger et al., Angewandte Chemie Int. Ed. Engl 49, 9995-9997 (2010)). In summary, the bacterial transglutaminase can be used for the conjugation of an amine group of the linker/drug to an acceptor glutamine residue of the antibody. Such acceptor glutamines can be introduced by engineering of the antibody by mutations or by generation of aglycosylated antibodies. Such aglycosylated antibodies can be generated by deglycosylation using N-glycosidase F (PNGaseF) or by mutation of the N297 (Kabat numbering) of the glycosylation site of the heavy chain to any other amino acid. Enzymatic conjugation of such aglycosylated antibodies was described for aglycosylated antibody variants bearing the mutations N297D, N297Q (Jeger et al., Angewandte Chemie Int. Ed. Engl 49, 9995-9997 (2010)), or N297S (see patent applications WO2013092998A1 and WO2013092983A2). Enzymatic conjugation using transglutaminase of such aglycosylated antibodies provides ADCs with DAR of 2 in general, in which both heavy chains are functionalized site specifically at position Q295 (Kabat numbering). The mutation N297Q of the antibody provides 1 additional site for conjugation at each heavy chain leading for example to ADCS with DAR of 4, in which both heavy chains are functionalized site-specifically at position Q295 and Q297 (Kabat numbering). Antibody variants bearing the mutations Q295N and N297Q provide one acceptor glutamine residue at position Q297 (Simone Jeger, Site specific conjugation of tumour targeting antibodies using transglutaminase, Dissertation at ETH Zurich (2009)). There are several examples in literature describing site specific conjugation of aglycosylated antibodies via transglutaminase (e.g. Dennler et al., Bioconjugate Chemistry 19, 569-578 (2014); Lhospice et al., Molecular Pharmaceutics 12, 1863-1871 (2015)).

### Methods of Use

The conjugates and compounds according to the invention show a valuable pharmacological and pharmacokinetic spectrum of action which could not have been predicted.

They are therefore suitable for use as medicaments for the treatment and/or prophylaxis of disorders in humans and animals.

Within the scope of the present invention, the term “treatment” includes prophylaxis.

### Commercial Utility

The conjugates and compounds of the present invention have surprisingly been found to effectively inhibit NAMPT finally resulting in cell death e.g. apoptosis and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by NAMPT, such as, for example, benign and malignant neoplasia, more specifically haematological tumours, solid tumors, and/or metastases thereof, e.g. leukaemias and myelodysplastic syndrome, malignant lymphomas, head and neck tumors including brain tumors and brain metastases, tumors of the thorax including non-small cell and small cell lung tumors, gastrointestinal tumors, endocrine tumors, mammary and other gynaecological tumors, urological tumours including renal, bladder and prostate tumours, skin tumors, and sarcomas, and/or metastases thereof, especially haematological tumors, solid tumours, and/or metastases of breast, bladder, bone, brain, central and peripheral nervous system, cervix, colon, endocrine glands (e.g. thyroid and adrenal cortex), endocrine tumours, endometrium, esophagus, gastrointestinal tumors, germ cells, kidney, liver, lung, larynx and hypopharynx, mesothelioma, ovary, pancreas, prostate, rectum, renal, small intestine, soft tissue, stomach, skin, testis, ureter, vagina and vulva as well as malignant neoplasias including primary tumors in said organs and corresponding secondary tumors in distant organs (“tumor metastases”). Haematological tumors can e.g. be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma.

Also included are myelodysplastic syndrome, plasma cell neoplasia, paraneoplastic syndromes, and cancers of unknown primary site as well as AIDS related malignancies.

One aspect of the invention is the use of a conjugate or a compound described supra for the treatment of acute myeloid leukemia (AML), as well as a method of treatment of AML, comprising administering an effective amount of a conjugate or a compound of the present invention. Well known and often used cancer cell lines to study AML are eg. THP-1 (human monocytic leukemia cell line).

One aspect of the invention is the use of a conjugate or a compound described supra for the treatment of breast cancer, including HER2-positive breast cancer, as well as a method of treatment of breast cancer, including HER2-positive breast cancer, comprising administering an effective amount of a conjugate or a compound of the present invention. A well known and often used cancer cell lines to study breast cancer, including HER2-positive breast cancer is the MDA-MB-453 breast cancer cell line [Cailleau R. et al., In vitro 14 (11):911-915, 1978].

One aspect of the invention is the use of a conjugate or a compound described supra for the treatment of brain tumors, including glioblastomas, as well as a method of treatment of brain tumors, including glioblastomas, comprising administering an effective amount of a conjugate or a compound of the present invention. Well known and often used cancer cell lines to study glioblastoma are eg. U251 MG (formerly known as U-373 MG) glioblastoma astrocytoma cells (Pontén, J., Macintyre, E. H. Acta Pathol Microbiol Scand A. 74, 465-486, 1968).

One aspect of the invention is the use of a conjugate or a compound described supra for the treatment of non-Hodgkin's lymphoma (NHL), including Mantle cell lymphoma (MCL), as well as a method of treatment of NHL, including MCL, comprising administering an effective amount of a conjugate or a compound of the present invention. Well known and often used cancer cell lines to study NHL, including MCL are eg. REC-1 (human Mantle cell lymphoma (B cell non-Hodgkin's lymphoma)).

One aspect of the invention is the use of a conjugate or a compound described supra for the treatment of lung cancer, as well as a method of treatment of lung cancer, comprising administering an effective amount of a conjugate or a compound of the present invention. Well known and often used cancer cell lines to study lung cancer are eg. A549 (human lung epithelial cell).

In accordance with an aspect of the present invention therefore the invention relates to a conjugate or a compound described supra, or an N-oxide, a salt, a tautomer or a stereoisomer of said conjugate or compound, or a salt of said N-oxide, tautomer or stereoisomer particularly a pharmaceutically acceptable salt thereof, or a mixture of same, as described and defined herein, for use in the treatment or prophylaxis of a disease, especially for use in the treatment of a disease.

Another particular aspect of the present invention is therefore the use of a conjugate or a compound described supra, or a stereoisomer, a tautomer, an N-oxide, a hydrate, a solvate, or a salt thereof, particularly a pharmaceutically acceptable salt thereof, or a mixture of same, for the prophylaxis or treatment of hyperproliferative diseases and/or disorders responsive to induction of cell death i.e apoptosis.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment of hyperproliferative diseases and/or disorders responsive to induction of cell death, wherein the diseases are cancer diseases, haematological tumours, solid tumors and/or metastases thereof, e.g. leukaemias and myelodysplastic syndrome, malignant lymphomas, head and neck tumors including brain tumors and brain metastases, tumors of the thorax including non-small cell and small cell lung tumors, gastrointestinal tumors, endocrine tumors, mammary and other gynaecological tumors, urological tumors including renal, bladder and prostate tumors, skin tumors, and sarcomas, and/or metastases thereof.

A preferred aspect is the use of a conjugate or a compound described supra for the prophylaxis and/or treatment of acute myeloid leukemia (AML), non-Hodgkin's lymphoma (particularly Mantle cell lymphoma), breast cancer (particularly HER2-positive breast), brain tumors (particularly glioblastoma) and lung cancer, and/or metastases thereof.

Another aspect of the present invention is the use of a conjugate or a compound described supra or a stereoisomer, a tautomer, an N-oxide, a hydrate, a solvate, or a salt thereof, particularly a pharmaceutically acceptable salt thereof, or a mixture of same, as described herein, in the manufacture of a medicament for the treatment or prophylaxis of a disease, wherein such disease is a hyperproliferative disorder or a disorder responsive to induction of cell death e.g. apoptosis. In an embodiment the disease is a haematological tumour, a solid tumour and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof. In a preferred embodiment the disease is acute myeloid leukemia (AML), non-Hodgkin's lymphoma (particularly Mantle cell lymphoma), breast cancer (particularly HER2-positive breast), brain tumors (particularly glioblastoma) and lung cancer, and/or metastases thereof.

### Method of Treating Hyper-Proliferative Disorders

The present invention relates to a method for using a conjugate or a compound described supra and compositions thereof, to treat mammalian hyper-proliferative disorders. Conjugates and compounds can be utilized to inhibit, block, reduce, decrease, etc., cell proliferation and/or cell division, and/or produce cell death e.g. apoptosis. This method comprises administering to a mammal in need thereof, including a human, an amount of a conjugate or a compound described supra, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus. Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Methods of Treating NAMPT Disorders

The present invention also provides methods for the treatment of disorders associated with aberrant NAMPT activity.

Such disorders include, but are not limited to, disorders associated with activation of NF-κB, inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, including, but not limited to, breast, prostate, lung, colon, cervix, ovary, skin, CNS, bladder, pancreas, leukaemia, lymphoma or Hodgkin's disease, cachexia, inflammation associated with infection and certain viral infections, including Acquired Immune Deficiency Syndrome (AIDS), adult respiratory distress syndrome, and ataxia telengiectasia, heart failure, hepatomegaly, cardiomegaly, diabetes, cystic fibrosis, symptoms of xenograft rejections, septic shock or asthma.

Involvement of NAMPT in the treatment of cancer is described in WO 97/48696. Involvement of NAMPT in immuno-suppression is described in WO 97/48397. Involvement of NAMPT for the treatment of diseases involving angiogenesis is described in WO2003/80054. Involvement of NAMPT for the treatment of rheumatoid arthritis and septic shock is described in WO 2008/025857. Involvement of NAMPT for the prophylaxis and treatment of ischaemia is described in WO 2009/109610.

Effective amounts of conjugates or compounds of the present invention can be used to treat such disorders, including those diseases (e.g., cancer) mentioned in the Background section above. Nonetheless, such cancers and other diseases can be treated with conjugates or compounds of the present invention, regardless of the mechanism of action and/or the relationship between NAMPT and the disorder.

The phrase “aberrant NAMPT activity” includes any abnormal expression or activity of the gene encoding the enzyme or of the polypeptide it encodes. Examples of such aberrant activity, include, but are not limited to, over-expression of the gene or polypeptide; gene amplification; mutations which produce constitutively-active or hyperactive enzyme activity; gene mutations, deletions, substitutions, additions, etc.

The present invention also provides for methods of inhibiting a NAMPT activity, comprising administering an effective amount of a conjugate or a compound of the present invention, including salts, polymorphs, metabolites, hydrates, solvates, prodrugs (e.g.: esters) thereof, and diastereoisomeric forms thereof. NAMPT activity can be inhibited in cells (e.g., in vitro), or in the cells of a mammalian subject, especially a human patient in need of treatment.

### Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, conjugates and compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death e.g. apoptosis of such cell types.

Preferably, the diseases of said method are haematological tumours, solid tumour and/or metastases thereof.

The conjugates and compounds of the present invention can be used in particular in therapy and prevention i.e. prophylaxis, especially in therapy of tumour growth and metastases, especially in solid tumours of all indications and stages with or without pre-treatment of the tumour growth.

Methods for Treating a Patient Diagnosed with or Suspected to have a Cancer Deficient in Nicotinic Acid Pathway

The present invention also provides methods for treating a patient diagnosed with or suspected to have a cancer deficient in nicotinic acid pathway.

Said method comprises the steps of administering to the patient:

(a) an effective amount of a conjugate or a compound of the present invention; and
(b) an effective amount of nicotinic acid.

Optionally, the method further comprises a step of administering to the patient:

(c) a DNA damaging agent.

Methods to determine whether a cancer is deficient in nicotinic acid pathway are known to the skilled person (for example in WO2009/072004 which is incorporated herein in its entirety by reference).

Suitable DNA damaging agents include, but are not limited to those described herein. The specific DNA damaging agent for each patient will vary according to the nature and severity of the condition as determined by the attending diagnostician, the activity of the specific conjugate or compound employed, the age and general condition of the patient, time of administration, route of administration, rate of excretion of the drug, drug combinations, and the like.

The present invention also includes a conjugate or a compound of the present invention in combination with nicotinic acid for use in the treatment of a patient diagnosed with or suspected to have a cancer deficient in nicotinic acid pathway.

The present invention also includes the use of a conjugate or a compound of the present invention in combination with nicotinic acid, in the manufacture of a medicament for the treatment of a patient diagnosed with or suspected to have a cancer deficient in nicotinic acid pathway.

In an embodiment the effective amount of nicotinic acid is administered intravenously.

In another embodiment the effective amount of nicotinic acid is administered orally.

In an embodiment the nicotinic acid is administered prior or subsequent to the administration of the conjugate or compound of the present invention.

In an embodiment the nicotinic acid is administered simultaneously with the administration of the conjugate or compound of the present invention.

Method for Predicting the Utility of Administering Nicotinic Acid to Reduce the Severity of Side-Effects of Cancer Treatment with NAMPT Inhibitors

The present invention also provides a method for stratifying patients according to the utility of administering nicotinic acid to reduce the severity of side-effects of cancer treatment with NAMPT inhibitors according to the present invention, the method comprising the steps of:

a) determining the level of Nicotinic acid phosphoribosyltransferase (NAPRT) in a cancer of said subject; and
b) 1) in the event of a level of NAPRT, as determined in step a) above, which is lower than a predetermined threshold value, selecting said subject for sequential or simultaneous treatment with i) an effective amount of a conjugate or a compound described supra, and ii) an effective amount of nicotinic acid, a nicotinic acid precursor or a prodrug of nicotinic acid; and
2) in the event of a level of NAPRT, as determined in step a) above, which is higher than or equal to a predetermined threshold value, selecting said subject for treatment with i) an effective amount of a conjugate or a compound described supra in the absence of sequential or simultaneous treatment with ii) an effective amount of nicotinic acid, a nicotinic acid precursor or a prodrug of nicotinic acid.

The invention further comprises a method for the treatment or for alleviating the symptoms of a cancer in a subject, the method comprising the steps of:

a) determining the level of Nicotinic acid phosphoribosyltransferase (NAPRT) in said subject; and,
b) 1) in the event of a level of NAPRT, as determined in step a) above, which is lower than a predetermined threshold value, treating said subject sequentially or simultaneous with i) a conjugate or a compound described supra, and ii) an effective amount of nicotinic acid, a nicotinic acid precursor or a prodrug of nicotinic acid; and
2) in the event of a level of NAPRT, as determined in step a) above, which is higher than or equal to a predetermined threshold value, treating said subject with i) a conjugate or a compound described supra in the absence of sequential or simultaneous treatment with ii) an effective amount of a nicotinic acid, a nicotinic acid precursor or a prodrug of nicotinic acid.

The above methods optionally comprise a further step c): administering said subject an effective amount of a DNA damaging agent.

In an embodiment of the above methods the level of NAPRT is determined in the tumour tissue of said subject.

In an embodiment of the above methods the level of nicotinic acid phosphoribosyltransferase (NAPRT) is determined on the level of nucleic acids encoding NAPRT, such as by RT-PCR or quantification of DNA methylation (suitable methods are provided, for example, in Shames et al., Clin Cancer Res; 19(24); 6912-23, which is incorporated herein in its entirety by reference).

In an embodiment of the above methods the level of Nicotinic acid phosphoribosyltransferase (NAPRT) is determined on the level of proteins, such as in assays based on specific antibodies or other specific binding partners to NAPRT.

The present invention also relates to a conjugate or a compound according to the present invention for use in a method for the treatment or for alleviating the symptoms of a cancer in a subject, said method comprising the steps a) and b) supra.

The present invention also relates to NAPRT for use as a biomarker useful in a method for predicting the utility of administering a nicotinic acid, a nicotinic acid precursor or a prodrug of nicotinic acid to reduce the severity of side-effects of cancer treatment with a conjugate or a compound according to the present invention.

The present invention further relates to the use of NAPRT as a biomarker in selecting responsive patients to the sequential or simultaneous treatment with i) an effective amount of a conjugate or a compound described supra, and ii) an effective amount of a nicotinic acid, a nicotinic acid precursor or a prodrug of nicotinic acid.

The present invention further relates to the use of Nicotinic acid phosphoribosyltransferase (NAPRT) as a biomarker in selecting patients that benefit from being treated with an effective amount of a conjugate or a compound described supra in the absence of sequential or simultaneous treatment with an effective amount of a nicotinic acid, a nicotinic acid precursor or a prodrug of nicotinic acid.

The stratification of the subjects is based on a predetermined threshold value. Suitable methods to establish the predetermined threshold value are know to the skilled person (for example in WO2011006988 or in U.S. Pat. No. 8,912,184 which are incorporated herein in their entirety by reference). The predetermined threshold value may be, for example, set by the medical practitioner based data from a plurality of patients, e.g. at least 5 patient, or at least 20 patient, or even at least 50 patients. Hence, in order to create basis for setting the threshold value, it will be necessary to first establish or obtain data from a cohort of existing patients to determine the level of NAPRT in their tumour tissue. The level of NAPRT in tumour tissue may be determined by one of a number of methods which either directly measure NAPRT, or which in a more indirect manner correlates (or is expected to correlate) with the level of NAPRT in the tissue in question. The cohort to which reference is made is desirably matched to one or more of tumour type, age, sex, or severity of disease, in particular the tumour type.

In one variant, however, the threshold value may set based on the level of NAPRT of a different tissue type than the tumour tissue in a population of human beings. This may be similar or identical patients, or may alternatively be healthy subjects. However, preferably, the threshold value is set based on the level of NAPRT in the same tissue, such as tumour tissue, as the tumour tissue in question, and obtained from plurality of patients with the same cancer indication.

### Methods of Sensitizing Cells to Radiation

In a distinct embodiment of the present invention, a conjugate or a compound of the present invention may be used to sensitize a cell to radiation. That is, treatment of a cell with a conjugate or a compound of the present invention prior to radiation treatment of the cell renders the cell more susceptible to DNA damage and cell death than the cell would be in the absence of any treatment with a conjugate or a compound of the invention. In one aspect, the cell is treated with at least one conjugate or compound of the invention.

Thus, the present invention also provides a method of killing a cell, wherein a cell is administered one or more conjugates or compounds of the invention in combination with conventional radiation therapy.

The present invention also provides a method of rendering a cell more susceptible to cell death, wherein the cell is treated one or more conjugates or compounds of the invention prior to the treatment of the cell to cause or induce cell death. In one aspect, after the cell is treated with one or more conjugates or compounds of the invention, the cell is treated with at least one conjugate or compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more conjugates or compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a conjugate or a compound of the invention is administered to a cell prior to the radiation or other induction of DNA damage in the cell. In another aspect of the invention, a conjugate or a compound of the invention is administered to a cell concomitantly with the radiation or other induction of DNA damage in the cell. In yet another aspect of the invention, a conjugate or a compound of the invention is administered to a cell immediately after radiation or other induction of DNA damage in the cell has begun.

In another aspect, the cell is in vitro. In another embodiment, the cell is in vivo.

### Pharmaceutical Compositions of the Compounds of the Invention

This invention also relates to pharmaceutical compositions containing one or more conjugates or compounds of the present invention. These compositions can be utilised to achieve the desired pharmacological effect by administration to a patient in need thereof. A patient, for the purpose of this invention, is a mammal, including a human, in need of treatment for the particular condition or disease.

Therefore, the present invention includes pharmaceutical compositions comprising a conjugate or a compound as defined supra, together with at least one pharmaceutically acceptable carrier or auxiliary.

Another aspect of the invention is a pharmaceutical composition comprising a pharmaceutically acceptable carrier or auxiliary and a pharmaceutically effective amount of a conjugate or a compound, or salt thereof, of the present invention.

Another aspect of the invention is a pharmaceutical composition comprising a pharmaceutically effective amount of a conjugate or a compound as defined supra and a pharmaceutically acceptable auxiliary for the treatment of a disease mentioned supra, especially for the treatment of haematological tumours, solid tumours and/or metastases thereof.

Another aspect of the invention is a pharmaceutical composition comprising a conjugate or a compound as defined supra and a pharmaceutically acceptable auxiliary for the treatment of a haematological tumour, a solid tumour and/or metastases thereof.

A pharmaceutically acceptable carrier or auxiliary is preferably a carrier that is non-toxic and innocuous to a patient at concentrations consistent with effective activity of the active ingredient so that any side effects ascribable to the carrier do not vitiate the beneficial effects of the active ingredient. Carriers and auxiliaries are all kinds of additives assisting the composition to be suitable for administration.

A pharmaceutically effective amount of a conjugate or a compound is preferably that amount which produces a result or exerts the intended influence on the particular condition being treated.

The conjugates and compounds of the present invention can be administered with pharmaceutically-acceptable carriers or auxiliaries well known in the art using any effective conventional dosage unit forms, including immediate, slow and timed release preparations.

The conjugates and compounds of this invention may be administered parenterally, that is, subcutaneously, intravenously, intraocularly, intrasynovially, intramuscularly, or interperitoneally, as injectable dosages of the conjugate or compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Controlled release formulations for parenteral administration include liposomal, polymeric microsphere and polymeric gel formulations that are known in the art.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Pharmaceutical compositions according to the present invention can be illustrated as follows:

i.v. Solution:

The conjugate according to the invention is dissolved in a concentration below the saturation solubility in a physiologically acceptable solvent (e.g. isotonic saline solution, D-PBS, or a formulation with glycine and sodium chloride in citrate buffer with addition of polysorbate 80). The solution is subjected to sterile filtration and dispensed into sterile and pyrogen-free injection vessels.

i.v. Solution:

The conjugates according to the invention can be converted to the administration forms mentioned. This can be accomplished in a manner known per se by “mixing with” or “dissolving in” inert, non-toxic, pharmaceutically suitable excipients (e.g. buffer substances, stabilizers, solubilizers, preservatives). The following, for example, may be present: amino acids (glycine, histidine, methionine, arginine, lysine, leucine, isoleucine, threonine, glutamic acid, phenylalanine and others), sugars and related compounds (glucose, saccharose, mannitol, trehalose, sucrose, mannose, lactose, sorbitol), glycerol, sodium salts, potassium, ammonium salts and calcium salts (e.g. sodium chloride, potassium chloride or disodiumhydrogenphosphate and many others), acetate/acetic acid buffer systems, phosphate buffer systems, citric acid and citrate buffer systems, trometamol (TRIS and TRIS salts), Polysorbates (e.g. Polysorbate 80 and Polysorbate 20), Poloxamers (e.g. Poloxamer 188 and Poloxamer 171), Macrogols (PEG derivatives, e.g. 3350), Triton X-100, EDTA salts, glutathione, albumins (e.g. human), urea, benzyl alcohol, phenol, chlorocresol, metacresol, benzalkonium chloride and many others.

Lyophilizate for Subsequent Conversion into an i.v., s.c. or i.m. Solution:

Alternatively the compounds of the invention may be converted into a stable lyophilizate (possibly with the aid of abovementioned excipients) and, before being administered, reconstituted with a suitable solvent (e.g. injection-grade water, isotonic saline solution) and administered.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the conjugates and compounds of this invention can readily be determined for treatment of each desired indication. The amount of the active ingredient to be administered in the treatment of one of these conditions can vary widely according to such considerations as the particular conjugate or compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Combination Therapies

The conjugates and compounds of this invention can be administered as the sole pharmaceutical agent or in combination with one or more other pharmaceutical agents where the combination causes no unacceptable adverse effects. Those combined pharmaceutical agents can be other agents having antiproliferative effects such as for example for the treatment of haematological tumours, solid tumours and/or metastases thereof and/or agents for the treatment of undesired side effects. The present invention relates also to such combinations.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, especially (chemotherapeutic) anti-cancer agents as defined supra. The combination can be a non-fixed combination or a fixed-dose combination as the case may be.

Methods of testing for a particular pharmacological or pharmaceutical property are well known to persons skilled in the art.

Another embodiment of the invention are conjugates and compounds according to the claims as disclosed in the Claims section wherein the definitions are limited according to the preferred or more preferred definitions as disclosed below or specifically disclosed residues of the exemplified conjugates and compounds and subcombinations thereof.

The following examples illustrate the invention in greater detail, without restricting it. Further conjugates and compounds according to the invention, of which the preparation is not explicitly described, can be prepared in an analogous way.

The term “according to” within the experimental section is used in the sense that the procedure referred to is to be used “analogously to”.

### Experimental Part

The following table lists the abbreviations used in this paragraph and in the Intermediates and Examples section as far as they are not explained within the text body.

| Abbreviation | Meaning |
|---|---|
| DMSO | dimethyl sulfoxide |
| DME | 1,2-dimethoxyethane |
| NMR | nuclear magnetic resonance |
| DMF | N,N-dimethylformamide |
| MS | mass spectroscopy |
| Rₜ | retention time |
| HPLC, LC | high performance liquid chromatography |
| MPLC | medium pressure liquid chromatography |
| h | Hour |
| min | Minute |
| r.t. | Room temperature (18-25° C.) |
| ppm | Chemical shift δ in parts per million |
| br. | broad signal (in NMR data) |
| s | singlet |
| d | doublet |
| dd | doublet of doublet |
| m | multiplet |
| ELSD | Evaporative Light Scattering Detector |
| ESI | electrospray ionisation |
| pos | positive |
| neg | negative |
| DAD | Diode Array Detector |
| m/z | mass-to-charge ratio (in mass spectrum) |
| EtOAc | ethyl acetate |
| HATU | 1-[bis(dimethylamino)methylene]-1H-1,2,3-triazolo[4,5-b]pyridinium 3-oxid |
| | hexafluorophosphate |

### Amino Acid Abbreviations

Ala=Alanine
Arg=Arginine
Asn=Asparagine
Asp=Aspartic acid
Cys=Cysteine
Glu=Glutamic acid
Gln=Glutamine
Gly=Glycine
His=Histidine
IIe=Isoleucine
Leu=Leucine
Lys=Lysine
Met=Methionine
Phe=Phenylalanine
Pro=Proline
Ser=Serine
Thr=Threonine
Trp=Tryptophan
Tyr=Tyrosine
Cit=citrulline
Val=Valine

The compounds of the present invention optionally contain a cleavable group SG. As used herein, when SG is a peptide the amino acid residues which constitute said peptide are herein referred to indistinctly by its three letter code, amino acid or amino acid residue (e.g. -Ala-Val-, -alanine-valine- or -alanyl-valyl-). It is understood that a hyphen at the beginning and/or at the end of the amino acid residues mark the points of attachment to the rest of the molecule. Unless stated otherwise, said hyphen may be connected to the N-terminus or the C-terminus of the amino acid residue to which it connects. The present invention includes all such possible connecting modes (e.g. -Ala-Val- -alanine-valine- or -alanyl-valyl- includes both (C-terminus)-Ala-Val-(N-terminus) and (N-terminus)-Ala-Val-(C-terminus)). Similarly, whenever said hyphens are not present, it is understood that the end amino acid residues of the peptide chain may connect to the rest of the molecule via the N-terminus or the C-terminus of the end amino acid residues (e.g. when SG is Ala-Val-, alanine-valine- or -alanyl-valyl- it includes both (C-terminus)-Ala-Val-(N-terminus) and (N-terminus)-Ala-Val-(C-terminus)). Unless indicated otherwise, the present invention includes all such possible connecting modes.

Other abbreviations not specified herein have their meanings customary to the skilled person.

The various aspects of the invention described in this application are illustrated by the following examples which are not meant to limit in any way the full scope of the invention as described herein.

### Specific Experimental Descriptions

NMR peak forms in the following specific experimental descriptions are stated as they appear in the spectra, possible higher order effects have not been considered. Chemical shifts (δ) are given in ppm. The chemical shifts were corrected by setting the DMSO signal to 2.50 ppm unless otherwise stated.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Reactions employing microwave irradiation may be run with a Biotage Initator® microwave oven optionally equipped with a robotic unit. The reported reaction times employing microwave heating are intended to be understood as fixed reaction times after reaching the indicated reaction temperature. The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel (“SiO₂”) or Isolute® Flash NH₂ silica gel (“amine-coated SiO₂”) in combination with a Isolera autopurifier (Biotage) and eluents such as gradients of e.g. hexane/ethyl acetate or dichloromethane/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia. In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The purities of the intermediates and examples are ≥95% as judged by LC/MS or ¹H-NMR spectra if not stated otherwise.

The percentage yields reported in the following examples are based on the starting component that was used in the lowest molar amount. Air and moisture sensitive liquids and solutions were transferred via syringe or cannula, and introduced into reaction vessels through rubber septa. Commercial grade reagents and solvents were used without further purification. The term “concentrated under reduced pressure” refers to use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

### Analytical LC-MS Conditions

LC-MS-data given in the subsequent specific experimental descriptions refer (unless otherwise noted) to the following conditions:

### Methods:

Method 1: Instrument: Waters Acquity UPLC-MS SQD; column: Acquity UPLC BEH C18 1.7 50×2.1 mm; Eluent A: water+0.1% vol. formic acid (99%), Eluent B: acetonitrile; gradient: 0-1.6 min 1-99% B, 1.6-2.0 min 99% B; rate 0.8 mL/min; temperature: 60° C.; injection: 1 μl; DAD scan: 210-400 nm; ELSD.

Method 2: Instrument: Waters Acquity UPLC-MS SQD; column: Acquity UPLC BEH C18 1.7 50×2.1 mm; Eluent A: water+0.2% vol. NH3 (32%), Eluent B: acetonitrile; gradient: 0-1.6 min 1-99% B, 1.6-2.0 min 99% B; rate 0.8 mL/min; temperature: 60° C.; injection: 1 μl; DAD scan: 210-400 nm; ELSD.

Method 3: Instrument: Waters Acquity binary pump, QDA, PDA; column: CSH C18 1.7 μm 50×2.1 mm; Eluent A: water+0.1% vol. formic acid (99%), Eluent B: acetonitrile+0.1% vol. formic acid (99%); gradient: 0-1.6 min 2-95% B, 1.2-1.4 min 95% B; rate 0.8 mL/min; temperature: 40° C.; injection: 2 μl.

Method 4: Instrument: Waters Acquity binary pump, QDA, PDA; column: CSH C18 1.7 μm 50×2.1 mm; Eluent A: water+0.1% vol. formic acid (99%), Eluent B: acetonitrile+0.1% vol. formic acid (99%); gradient: 0-4.0 min 2-95% B, 4.0-4.4 min 95% B; rate 0.8 mL/min; temperature: 40° C.; injection: 2 μl; Method 5: Instrument: Waters Acquity quaternary pump, QDA, PDA; column: XBridge BEH C18 2.5 μm 50×2.1 mm; Eluent A: 10 mM ammonium bicarbonate pH 10, Eluent B: acetonitrile; gradient: 0-1.2 min 2-98% B, 1.2-1.4 min 98% B; rate 0.8 mL/min; temperature: 40° C.; injection: 2 μl; Method 6: Instrument: Waters Acquity quaternary pump, QDA, PDA; column: XBridge BEH C18 2.5 μm 50×2.1 mm; Eluent A: 10 mM ammonium bicarbonate pH 10, Eluent B: acetonitrile; gradient: 0-4.0 min 2-98% B, 4.0-4.6 min 98% B; rate 0.8 mL/min; temperature: 40° C.; injection: 2 μl;

### Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

### Preparation of Antibody/Active Compound Conjugates (ADC)

### General Process for Generating of Different Anti-Target Antibodies

### Humanization

The murine antibody sequence of the 7G3 antibody was humanized by transferring the CDRs into a human antibody skeleton. For the definition of the CDRs according to Kabat, see Andre C. R. Martin, “Protein sequence and structure analysis of antibody variable domains” in Antibody Engineering (Springer Lab Manuals), Eds.: Duebel, S. and Kontermann, R., Springer-Verlag, Heidelberg. After comparison of the murine frame sequences (without CDRs) with human germline sequences, a similar frequently occurring human frame sequence was selected. In this case, it was the heavy chain IGHV1-46-01 with the J sequence IGHJ4-03 and the light chain IGKV4-1-01 with the J segment IGKJ2. The germline sequences originated from the VBASE2 database (Retter I, Althaus H H, Münch R, Müller W: VBASE2, an integrative V gene database. Nucleic Acids Res. 2005 Jan. 1; 33(Database issue):D671-4). The sequences were named using the IMGT system (Lefranc, M.-P., Giudicelli, V., Ginestoux, C., Jabado-Michaloud, J., Folch, G., Bellahcene, F., Wu, Y., Gemrot, E., Brochet, X., Lane, J., Regnier, L., Ehrenmann, F., Lefranc, G. and Duroux, P. IMGT®, the international ImMunoGeneTics information System®. Nucl. Acids Res, 37, D1006-D1012 (2009); doi:10.1093/nar/gkn838). The antibody variant TPP-5969 described herein carries various point mutations differing from the human germline sequence which may influence its properties.

Further anti-B7H3 antibodies were generated, for example, by screening of a phage display library for recombinant murine B7H3 (murine CD276; Gene ID: 102657) and human B7H3 (human CD276; Gene ID: 80381) expressing cells. The antibodies obtained in this manner were reformatted into the human IgG1 format and used for the working examples described here. In addition, antibodies which bind to B7H3 are known to the person skilled in the art.

### General Process for Expressing Anti-Target Antibodies in Mammalian Cells

The antibodies, for example TPP-8382, TPP-509, TPP-668, TPP-9574 and TPP-1015 were produced in transient mammalian cell cultures as described by Tom et al., Chapter 12 in Methods Express: Expression Systems edited by Michael R. Dyson and Yves Durocher, Scion Publishing Ltd, 2007.

General Process for Purifying Antibodies from Cell Supernatants

The antibodies, for example TPP-8382, TPP-509, TPP-668, TPP-9574 and TPP-1015 were obtained from the cell culture supernatants. The cell supernatants were clarified by centrifugation of cells. The cell supernatant was then purified by affinity chromatography on a MabSelect Sure (GE Healthcare) chromatography column. To this end, the column was equilibrated in DPBS pH 7.4 (Sigma/Aldrich), the cell supernatant was applied and the column was washed with about 10 column volumes of DPBS pH 7.4+500 mM sodium chloride. The antibodies were eluted in 50 mM sodium acetate pH 3.5+500 mM sodium chloride and then purified further by gel filtration chromatography on a Superdex 200 column (GE Healthcare) in DPBS pH 7.4.

### Checking the Antigen-Binding of the ADCs

The capability of the binder of binding to the target molecule was checked after coupling had taken place. The person skilled in the art is familiar with multifarious methods which can be used for this purpose; for example, the affinity of the conjugate can be checked using ELISA technology or surface plasmon resonance analysis (BIAcore™ measurement). The conjugate concentration can be measured by the person skilled in the art using customary methods, for example for antibody conjugates by protein determination. (see also Doronina et al.; Nature Biotechnol. 2003; 21:778-784 and Polson et al., Blood 2007; 1102:616-623).

The following antibodies were used for the coupling reactions:

| Antibody | Abbreviation |
|---|---|
| Anti-HER2 TPP-1015 | A |
| Anti-CXCR5 TPP-9574 | B |
| Anti-B7H3 TPP-8382 | C |
| Anti-C4.4a TPP-509 | D |
| Anti-C4.4a TPP-668 | E |

The letter in the ADC example number describes the respective antibody part. The letter “M” denotes the respective active metabolite derived from the ADC after intracellular degradation of the antibody and/or linker part of the ADC. In case several preparations have been made of a certain antibody-drug conjugate the different preparations are distinguished by an additional small case letter (a, b, c, etc.). For illustration, example 36Ca refers to preparation “a” of an anti-B7H3 TPP-8382-conjugate whereas example 36Cb refers to preparation “b” of the same anti-B7H3 TPP-8382-conjugate.

### Procedure 1: General Process for Coupling to Cysteine Side Chains

Between 2 and 8 equivalents of tris(2-carboxyethyl)phosphine hydrochloride (TCEP), dissolved in PBS buffer, were added to a solution of the appropriate antibody in PBS buffer in the concentration range between 1 mg/mL and 20 mg/mL, preferably in the range of about 10 mg/mL to 15 mg/mL, and the mixture was stirred at r.t. for 1 h. For this purpose, the solution of the respective antibody used can be employed at the concentrations stated in the working examples, or it may optionally also be diluted with PBS buffer to about half of the stated starting concentrations in order to get into the preferred concentration range. Subsequently, depending on the intended loading from 2 to 20 equivalents, preferably about 5-16 equivalents of the maleinimide precursor compound or halide precursor compound to be coupled can be added as a solution in DMSO. Here, the amount of DMSO should not exceed 10% of the total volume. The reaction was stirred in the case of maleinimide precursors for 60-240 min at r.t. and then applied to PBS-equilibrated PD 10 columns (Sephadex® G-25, GE Healthcare) and eluted with PBS buffer. Generally, unless indicated otherwise, 5 mg of the antibody in question in PBS buffer were used for the reduction and the subsequent coupling. Purification on the PD10 column thus in each case afforded solutions of the respective ADCs in 3.5 mL PBS buffer. The sample was then concentrated by ultracentrifugation and optionally rediluted with PBS buffer. If required, for better removal of low-molecular weight components, concentration by ultrafiltration was repeated after redilution with PBS buffer. For biological tests, if required, the concentrations of the final ADC samples were optionally adjusted to the range of 0.5-15 mg/mL by redilution. The respective protein concentrations, stated in the working examples, of the ADC solutions were determined. Furthermore, antibody loading (drug/mAb ratio) was determined using the methods described under procedure 5.

Typically 3.0 equivalents of tris(2-carboxyethyl)phosphine hydrochloride (TCEP) and 8.0 equivalents of the corresponding maleimide were employed in the conjugation reaction. For the generation of species with a higher drug/antibody ratio (>4) typically 8.0 equivalents of tris(2-carboxyethyl)phosphine hydrochloride (TCEP) and 16.0 equivalents of the corresponding maleimide were employed in the conjugation reaction.

Typically the following general procedure was used for the conjugation of 5 mg of antibody:

Under argon, a solution of 0.029 mg of TCEP in 50 μl of PBS buffer (pH 7.2) was added to 5 mg of the antibody in question in 0.5 mL of PBS buffer (pH 7.2) (c˜10 mg/mL). The reaction was stirred at r.t. for 30 min, and 0.27 μmol of the maleinimide precursor compound dissolved in 50 μl of DMSO were then added. After a further 1.5 h-2 h of stirring at r.t., the reaction was diluted with 1900 μl of PBS buffer (pH 7.2). This solution was then applied to PD 10 columns (Sephadex G-25, GE Healthcare) which had been equilibrated with PBS buffer (pH 7.2) and was eluted with PBS buffer (pH 7.2). The eluate was then centrifuged at 10° C./4G for 5 min, and the supernatant was then concentrated by ultracentrifugation and rediluted with PBS buffer (pH 7.2) to a volume of 2.5 mL.

Typically the following general procedure was used for the conjugation of 35 mg of antibody:

Under argon, a solution of 0.201 mg of TCEP in 200 μl of PBS buffer (pH 7.2) was added to 35 mg of the antibody in question in 2.5 of PBS buffer (pH 7.2) (c˜15 mg/mL). The reaction was stirred at r.t. for 30 min. and 1.17 μmol of the maleinimide precursor compound dissolved in 200 μl of DMSO were then added. After a further 1.5 h-2 h of stirring at r.t., the reaction was diluted with PBS buffer (pH 7.2) to a total volume of 5.0 mL. This solution was then applied to PD 10 columns (Sephadex G-25, GE Healthcare) which had been equilibrated with PBS buffer (pH 7.2) and was eluted with PBS buffer (pH 7.2). The eluate was then centrifuged at 10° C./4G for 5 min, and the supernatant was then concentrated by ultracentrifugation and rediluted with PBS buffer (pH 7.2) to a volume of 3.5 mL.

Unless indicated otherwise, the immunoconjugates shown in the examples were prepared by this process. Depending on the linker, the ADCs shown in the examples may also be present to a lesser or higher degree in the form of the hydrolysed open-chain succinamides attached to the antibodies. Both isomeric forms could be present.

Such ADCs in which the linker is attached to the antibodies through hydrolysed open-chain succinamides may optionally also be prepared in a targeted manner by an exemplary procedure as follows:

Procedure 2: General Process for Coupling to Cysteine Side Chains with Ring-Opened Maleimide

Typically the following general procedure was used:

Under argon, a solution of 0.029 mg of TCEP in 50 μl of PBS buffer (pH 7.2) was added to 5 mg of the antibody in question in 0.5 mL of PBS buffer (pH 8) (c˜10 mg/mL). The reaction was stirred at r.t. for 30 min, and 0.27 μmol of the maleinimide precursor compound dissolved in 50 μl of DMSO were then added. After a further 1.5 h-2 h of stirring at r.t., the reaction was diluted with 1900 μl of PBS buffer (pH 8). This solution was then applied to PD 10 columns (Sephadex® G-25, GE Healthcare) which had been equilibrated with PBS buffer pH 8 and was eluted with PBS buffer pH 8. The eluate was then stirred at r.t. under argon overnight. After that the eluate was then centrifuged at 10° C./4G for 5 min, and the supernatant was then concentrated by ultracentrifugation and rediluted with PBS buffer (pH 7.2) to a volume of 2.5 mL. The respective protein concentrations, stated in the working examples, of the ADC solutions were determined. Furthermore, antibody loading (drug/mAb ratio) was determined using the methods described under procedure 5.

Other potentially hydrolysis-sensitive thianylsuccinimide bridges to the antibody in the working examples contain the following linker substructures, where #1 represents the thioether linkage to the antibody and #2 the point of attachment to the modified payload:

These linker substructures represent the linking unit to the antibody and have (in addition to the linker composition) a significant effect on the structure and the profile of the metabolites formed in the tumour cells.

### Procedure 3: General Process for Coupling to Lysine Side Chains

To a solution of the appropriate antibody in PBS buffer in the concentration range between 1 mg/mL and 20 mg/mL, preferably in the range of about 10 mg/mL to 15 mg/mL, was added a solution of the corresponding final intermediate (e.g. N-hydroxysuccinimidyl ester), typically 2-10 equivalents, in DMSO. After stirring for 30 min to 6 h at r.t. again a solution of the corresponding final intermediate (e.g. N-hydroxysuccinimidyl ester), typically 2-10 equivalents, in DMSO was added and the mixture was stirred for further 30 min to 6 h at r.t. Preferentially, the amount of added DMSO should not exceed 10% of the total volume. After that the mixture was applied to PBS-equilibrated PD 10 columns (Sephadex G-25, GE Healthcare) and eluted with PBS buffer. Generally, unless indicated otherwise, 5 mg of the antibody in question in PBS buffer were used for the reduction and the subsequent coupling. Purification on the PD10 column thus in each case afforded solutions of the respective ADCs in 3.5 mL PBS buffer. The sample was then concentrated by ultracentrifugation and optionally rediluted with PBS buffer. If required, for better removal of low-molecular weight components, concentration by ultrafiltration was repeated after redilution with PBS buffer. For biological tests, if required, the concentrations of the final ADC samples were optionally adjusted to the range of 0.5-15 mg/mL by redilution. The respective protein concentrations, stated in the working examples, of the ADC solutions were determined. Furthermore, antibody loading (drug/mAb ratio) was determined using the methods described under procedure 5.

Typically the following general procedure was used for the conjugation of 5 mg of antibody:

Under argon, a solution of 0.175 μmol of the final intermediate (e.g. N-hydroxysuccinimidyl ester) in 25 μl of DMSO was added to to 5 mg of the antibody in question in 0.5 mL of PBS buffer (pH 7.2) (c˜10 mg/mL). The reaction was stirred at r.t. for 1 h and again a solution of 0.175 μmol of the final intermediate in 25 μl of DMSO was added. After stirring for further 1 h the reaction was diluted with PBS buffer (pH 7.2) to a total volume of 2.5 mL. This solution was then applied to PD 10 columns (Sephadex G-25, GE Healthcare) which had been equilibrated with PBS buffer (pH 7.2) and was eluted with PBS buffer (pH 7.2). The eluate was then centrifuged at 10° C./4G for 5 min, and the supernatant was then concentrated by ultracentrifugation and rediluted with PBS buffer (pH 7.2) to a volume of 2.5 mL.

Typically the following general procedure was used for the conjugation of 35 mg of antibody:

Under argon, a solution of 1.17 μmol of the final intermediate (e.g. N-hydroxysuccinimidyl ester) in 300 μl of DMSO was added to to 35 mg of the antibody in question in 5 mL of PBS buffer (pH 7.2) (c˜7 mg/mL). The reaction was stirred at r.t. for 1 h and again a solution of 1.17 μmol of the final intermediate in 300 μl of DMSO was added. After stirring for further 1 h the reaction was diluted with PBS buffer (pH 7.2) to a total volume of 7.5 mL. This solution was then applied to PD 10 columns (Sephadex G-25, GE Healthcare) which had been equilibrated with PBS buffer (pH 7.2) and was eluted with PBS buffer (pH 7.2). The eluate was then centrifuged at 10° C./4G for 5 min, and the supernatant was then concentrated by ultracentrifugation and rediluted with PBS buffer (pH 7.2) to a volume of 3.5 mL.

Procedure 4: General Process for Coupling to Cysteine Side Chains in Case the Final Intermediate Bears a Reducible Moiety, e.g. Disulfide

In case the final intermediate bears a reducible moiety like e.g. disulfide, remaining TCEP from the antibody reduction step may also cleave the reducible moiety in the final intermediate and should thus been removed. The general methods for the conjugation to cysteine side chains should thus be adjusted. Typically the following general procedure was used:

Under argon, a solution of 0.029 mg of TCEP in 50 μl of PBS buffer (pH 7.2) was added to 5 mg of the antibody in question in 0.5 ml of PBS buffer (pH 8) (c˜10 mg/ml). The reaction was stirred at RT for 30 min and was then applied to PD 10 columns (Sephadex® G-25, GE Healthcare) which had been equilibrated with PBS buffer (pH 7.2) and was eluted with PBS buffer (pH 7.2). To the eluate 0.27 μmol of the maleinimide precursor compound dissolved in 50 μl of DMSO were then added. After a further 1.5 h-2 h of stirring at RT, the reaction was diluted with 1900 μl of PBS buffer (pH 8). This solution was then applied to PD 10 columns (Sephadex® G-25, GE Healthcare) which had been equilibrated with PBS buffer (pH 7.2) and was eluted with PBS buffer (pH 7.2). The eluate was then centrifuged at 10° C./4G for 5 min, and the supernatant was then concentrated by ultracentrifugation and rediluted with PBS buffer (pH 7.2) to a volume of 2.5 mL. This solution was then applied to PD 10 columns (Sephadex® G-25, GE Healthcare) which had been equilibrated with PBS buffer pH 8 and was eluted with PBS buffer pH 8. The eluate was then stirred at RT under argon overnight. After that the eluate was then centrifuged at 10° C./4G for 5 min, and the supernatant was then concentrated by ultracentrifugation and rediluted with PBS buffer (pH 7.2) to a volume of 2.5 mL. The respective protein concentrations, stated in the working examples, of the ADC solutions were determined. Furthermore, antibody loading (drug/mAb ratio) was determined using the methods described under procedure 5.

### Procedure 5: Determination of the Antibody Identity, the Toxophore Loading (DAR), and the ADC Concentration

For protein identification in addition to molecular weight determination after deglycosylation and/or denaturing, a tryptic digestion was carried out, which, after denaturing, reduction and derivatization, confirms the identity of the protein via the tryptic peptides found.

The toxophor loading of the conjugates described in the working example was determined as follows:

Determination of toxophor loading of lysine coupled ADCs (if not done by UV analytics, see below) was carried out by mass spectromic determination of the molecular weights of the individual conjugate species. Here, the conjugates were first deglycosylated using PNGaseF, acidified and after HPLC separation/desalting over a short C₄ column (GromSil 300 Butyl-1 ST, 5 μm, 5 mm×500 μm), analysed by mass spectrometry using an ESI-QTof System (Bruker Daltonik). All spectra over the signal in the TIC (Total Ion Chromatogram) were added and the molecular weight of the different conjugate species was calculated based on MaxEnt deconvolution. The DAR (=drug/antibody ratio) was then calculated out of the sum of toxophor number weighted coupled species divided by the sum of the singly weighted integration results of each species.

Determination of cysteine coupled ADCs (if not done by UV analytics, see below) was carried out by mass spectrometric determination of the molecular weights of the individual conjugate species after reduction. Guanidinium hydrochloride (GuHCl) (28.6 mg) and a solution of DL-Dithiothreitol (DTT) (500 mM, 3 μL) were added to the ADC solution (1 mg/mL, 50 μL). The mixture was incubated at 55° C. for 1 hour and then analyzed by mass spectrometry after online desalting using an ESI-QT of Bruker Daltonik. For DAR determination, all spectra over the signal in the TIC (Total Ion Chromatogram) were added and the molecular weight of the different conjugate species was calculated based on MaxEnt deconvolution of light and heavy Chain. Average loading of the antibody with toxophors was calculated from the peak areas determined by integration as double the sum of the HC-Load and the LC-load, whereas the HC-load is the sum of toxophor number weighted integration results of all heavy chain (HC)-peaks divided by the sum of the singly weighted integration results of the HC-peaks and whereas the LC-load is the sum of toxophor number weighted integration results of the light chain (LC)-peaks divided by the sum of the singly weighted integration results of all LC peaks.

For determination of the ring opening percentage of the cysteine adduct, the areas of the molecular weight peaks of the ring closed and the ring opened cysteine adduct (delta in molecular weight 18 Da) were determined for the 1-fold conjugated light chain (also possible for light and heavy chain). The mean value over all variants gives the percentage of ring opened cysteine adduct.

Alternatively, the Drug load was determined by UV-absorption during size exclusion chromatography (SEC). Here, 50 μL of the ADC solution were analyzed by size exclusion chromatography. The analysis was carried out on an Agilent 1260 HPLC system with detection at 280 nm and detection at a toxophore related wavelength. A Superdex 200 10/300 GL from GE Healthcare (Lot No: 10194037) (10×310 mm, 13 μm particle size) was used at a flow rate of 1 ml/min with using isocratic condition. Mobile phase consisted of PBS buffer (pH 7.2). For determination of the drug load out of the SEC profile, the ratio R of the peak area of the monomer peak at the drug specific wavelength (λ_{drug}) and at 280 nm was determined. Out of this ratio R, the DAR can be calculated as follows:

$$DAR = \frac{{ɛ}_{Ab}^{{λ}_{drug}} - R \cdot {ɛ}_{Ab}^{280}}{R \cdot {ɛ}_{D}^{280} - {ɛ}_{D}^{{λ}_{drug}}}$$

whereas ε stands for the molar extinction coefficients of the antibody (Ab) and the drug (D).

The extinction coefficients of the antibody at 280 nm and at the drug wavelength were determined experimentally. Mean values of different antibodies were used for calculation. For the Nampt toxophores (compounds without additional linker) the extinction coefficients were determined experimentally. The following wavelengths and extinction coefficients were determined and used for the DAR calculations:

| | | ε(280 nm) | ε(310 nm) | ε(314 nm) | |
|---|---|---|---|---|---|
| | (λ_{drug}) (nm) | [1/μM] | [1/μM] | [1/μM] | Examples |
| Antibody | | 0.2284 | 0 | 0 | |
| 5-Chinolin, | 310 | 0.045 | 0.030 | | 29C, 29D, |
| | | | | | 27C, 31C, |
| Aromat | | | | | 31D, 31E, |
| | | | | | 43D, 43E, |
| | | | | | 35C, 35D, |
| | | | | | 35A |
| 5-Chinolin | 314 | 0.014 | | 0.028 | 28D, 30C, |
| | | | | | 30D, 30F, |
| | | | | | 30C, 36C, |
| | | | | | 36D, 36E, |
| | | | | | 30A, 36A, |
| | | | | | 41A, 42E, |
| | | | | | 42A |
| 2-Oxadiazol, | 314 | 0.043 | | 0.028 | 34C, 34D, |
| Aromat | | | | | 34E |
| 2-Oxadiazol | 314 | 0.024 | | 0.033 | 33C |
| 7-Chinolin | 314 | 0.015 | | 0.031 | 46A, 47A, |
| | | | | | 47D, 48C, |
| | | | | | 48A, 48D |

Drug loads (Drug/mAb ratios) that have been determined via UV-absorption are marked with ″(UV)″ in the experimental part.

The concentration of ADCs was determined by measuring the absorption at 280 nm. The concentration was calculated using the absorption coefficient of the respective antibody. To take into account the absorbance of the toxophore at 280 nm, in some cases the concentration was corrected using the following equation:

concentration=preliminary concentration/((1+DAR_{UV}*(ε_{Toxophore 280 nm}/ε_{Antibody 280 nm}))

whereas “preliminary concentration” is the concentration being calculated only using the extinction coefficient of the antibody, DAR_{UV} is the drug load of the respective ADC determined by UV absorption, and ε_{Toxophore 280 nm} and ε_{Antibody 280 nm} are the extinction coefficients at 280 nm of the toxophore and the antibody respectively.

The concentration correction was done for the following examples:

### 35C, 35D, 36C, 36D, 36E, 30A, 35A, 36A, 41A, 42E, 42A

### Intermediate 1-1

### (2E)-3-(Quinolin-5-yl)-1-(4-nitrophenyl)prop-2-en-1-on

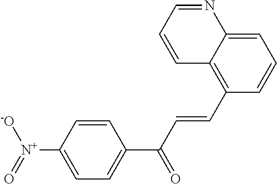

To 1-(4-nitrophenyl)ethanone (CAS 100-19-6, 3.00 g, 18.2 mmol) in THE (49 mL) and ethanol (240 mL) were added chinolin-5-carbaldehyde (CAS 22934-41-4, 3.43 g, 21.8 mmol) and ammonium acetate (1.34 g, 17.4 mmol). The mixture was heated to 70 C for 3 days. Then the mixture was cooled down and formic acid (69 mL, 1.8 mol) was added at 60 C. The mixture was stirred for 1 h until r.t. was reached. The formed precipitate was filtered off, washed with ethanol to give a first batch. The filtrate was concentrated under reduced pressure and ethanol (80 mL) and THE (12 mL) were added. The resulting mixture was stirred overnight, the formed precipitate was filtered off, washed with ethanol to give a second batch. The resulting filtrate was concentrated under reduced pressure and was purified by flash chromatography (SiO2, hexane/ethyl acetate gradient 0%-45%-70%) to give a third batch. All batches were combined to give 3.15 g (85% purity, 50% yield) of the title compound.

LC-MS (Method 1): Rₜ=1.12 min; MS (ESIpos): m/z=305 [M+H]⁺

¹H-NMR (400 MHz, DMSO-d₆) δ [ppm]: 7.66 (dd, 1H), 7.89 (t, 1H), 8.09 (d, 1H), 8.17 (d, 1H), 8.38-8.47 (m, 5H), 8.58 (d, 1H), 8.80 (d, 1H), 8.99 (dd, 1H).

### Intermediate 1-2

### 4-(4-Nitrophenyl)-4-oxo-2-(quinolin-5-yl)butanenitrile

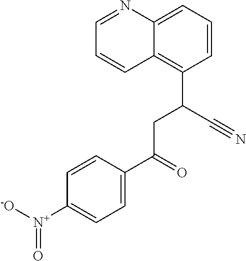

In a pressure tube (2E)-1-(4-nitrophenyl)-3-(quinolin-5-yl)prop-2-en-1-one (2.00 g, 6.57 mmol) was suspended in 1,4-dioxane (20 mL) and degassed with argon. Then cesium carbonate (10.7 mg, 32.9 μmol), trimethylsilyl cyanide (1.3 mL, 9.9 mmol) and water (590 μl) were added and degassed with argon again. The mixture was heated to 100° C. for 2.5 days, while after 2 h, 20 h and 2 days additional trimethylsilyl cyanide (each 0.4 mL, 3.3 mmol) was added. After cooling to r.t. the mixture was poured carefully on ice-water (200 mL) and extracted with ethyl acetate (4 times). The combined organic phases were washed with brine, filtered over a water separating filter and were concentrated under reduced pressure. The crude product was purified by column chromatography (SiO₂, hexane/ethyl acetate gradient 0%-100%) to give 682 mg (90% purity, 29% yield) of the title compound.

LC-MS (Method 2): Rt=1.06 min, MS (ESIpos): m/z=332 [M+H]⁺

¹H-NMR (400 MHz, DMSO-d₆) δ [ppm]: 4.01 (dd, 1H), 4.24 (dd, 1H), 5.37 (dd, 1H), 7.65 (dd, 1H), 7.83 (dd, 1H), 7.87-7.91 (m, 1H), 8.06 (d, 1H), 8.23-8.28 (m, 2H), 8.31-8.41 (m, 2H), 8.72 (d, 1H), 8.98 (dd, 1H).

### Intermediate 1-3

### 6-(4-Nitrophenyl)-4-(quinolin-5-yl)-4,5-dihydropyridazin-3(2H)-one

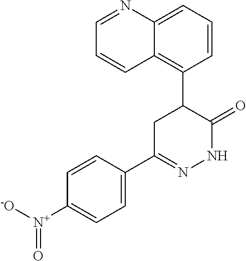

To a mixture of 4-(4-nitrophenyl)-4-oxo-2-(quinolin-5-yl)butanenitrile (650 mg, 1.96 mmol) in ethanol (6.9 mL) was added hydrazine hydrate (3.4 mL, 69 mmol) at 0 C and the mixture was heated to 50 C for 15 min. After that the mixture was cooled to r.t., poured on water (22 mL) and stirred for 30 min. The formed precipitate was filtered off and ethanol (22 mL) and water (22 mL) were added to the collected solid. The resulting mixture was heated to 90 C for 26 h. Then acetic acid (560 μl, 9.8 mmol) was added and the mixture was stirred for further 10 h at 90° C. After cooling to r.t. water (5 mL) was added to the mixture. The formed fine precipitate was filtered off and washed with water to give 405 mg (72% purity, 60% yield) of the title compound.

LC-MS (Method 2): Rt=0.95 min: MS (ESIpos): m/z=347 [M+H]⁺

¹H-NMR (400 MHz, DMSO-d₆) δ [ppm]: 3.41 (dd, 1H), 3.52 (m, 1H), 4.83 (dd, 1H), 7.51-7.60 (m, 2H), 7.76 (dd, 1H), 7.98 (d, 1H), 8.02-8.08 (m, 2H), 8.13-8.28 (m, 2H), 8.61 (d, 1H), 8.92 (dd, 1H), 11.58 (s, 1H).

### Intermediate 1-4

### 6-(4-Aminophenyl)-4-(quinolin-5-yl)-4,5-dihydropyridazin-3(2H)-one

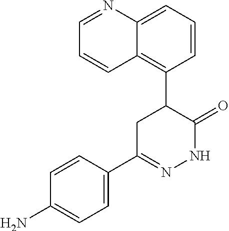 [SMILES: Nc1ccc(C2=NNC(=O)C(c3cccc4ncccc34)C2)cc1]

To 4-(4-nitrophenyl)-4-oxo-2-(quinolin-5-yl)butanenitrile (440 mg, 1.27 mmol) in methanol (13 mL) was added palladium on charcoal (68 mg, 10%). The mixture was stirred under a hydrogen atmosphere for 3 h at 50° C. and for 22 h at r. t. Additional palladium on charcoal (53 mg, 10%) was added and stirring was continued for 2 days. The hydrogen atmosphere was exchanged to argon and the mixture was filtered through a short path of celite, which afterwards was thoroughly washed with a warm ethyl acetate methanol mixture (1:1). The extraction of the celite with an ethylacetate methanol mixture at 60° C. was repeated to yield 400 mg (67% purity, 67% yield) of the title compound containing 6-(4-nitrophenyl)-4-(quinolin-5-yl)-pyridazin-3(2H)-one as minor impurity.

LC-MS (Method 2): Rt=0.73 min; MS (ESpos): m/z=317 [M+H]⁺

¹H-NMR (400 MHz, DMSO-d₆) δ [ppm]: 3.19 (dd, 1H), 3.31 (dd, 1H), 4.64 (dd, 1H), 5.50 (s, 2H), 6.50-6.55 (m, 2H), 7.44-7.51 (m, 3H), 7.55 (dd, 1H), 7.72 (dd, 1H), 7.95 (d, 1H), 8.61 (d, 1H), 8.91 (dd, 1H), 11.01 (s, 1H).

### Intermediate 1-5

### 4-Nitrophenyl {4-[6-oxo-5-(quinolin-5-yl)-1,4,5,6-tetrahydropyridazin-3-yl]phenyl}carbamate

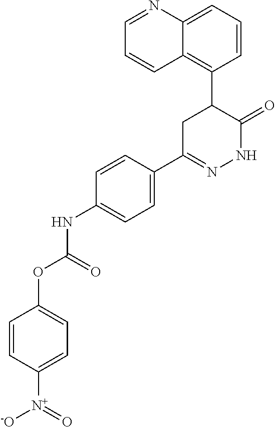

To 6-(4-aminophenyl)-4-(quinolin-5-yl)-4,5-dihydropyridazin-3(2H)-one (234 mg, 740 μmol) in THE (15 mL) was added at r.t. 4-nitrophenyl carbonochloridate (CAS-No. 7693-46-1, 298 mg, 1.48 mmol) and the mixture was heated to 60° C. After 1 h the addition of 4-nitrophenyl carbonochloridate (298 mg, 1.48 mmol) was repeated and stirring at 60° C. was continued for 30 min. After cooling to r.t. the mixture was concentrated under reduced pressure to give 830 mg (50% purity, quant.) of the raw title compound, which was directly used in the next step.

LC-MS (Method 1): Rt=0.96 min; MS (ESpos): m/z=482 [M+H]⁺.

### Example 1

### N-{4-[6-Oxo-5-(quinolin-5-yl)-1,4,5,6-tetrahydropyridazin-3-yl]phenyl}-1,3-dihydro-2H-pyrrolo[3,4-c]pyridine-2-carboxamide

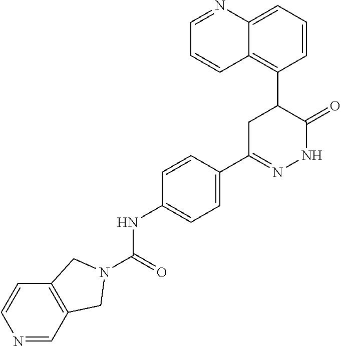 [SMILES: O=C1NN=C(c2ccc(NC(=O)N3Cc4ccncc4C3)cc2)CC1c1cccc2ncccc12]

2,3-Dihydro-1H-pyrrolo[3,4-c]pyridine dihydrochloride (CAS-No. 6000-50-6, 214 mg, 1.11 mmol) was added to a suspension of raw 4-nitrophenyl {4-[6-oxo-5-(quinolin-5-yl)-1,4,5,6-tetrahydropyridazin-3-yl]phenyl}carbamate (356 mg, 740 μmol) in dichloromethane (14 mL) and N,N-diisopropylethylamine (640 μl, 3.7 mmol). After stirring at r. t. for 2 h the mixture was cooled to −20° C. and the precipitate was filtered off after standing for 12 h and washed with cold dichloromethane. The precipitate was stirred in aqueous sodium hydroxide (20 mL, 1M), filtered, washed with water and dried under reduced pressure to give 175 mg (67% purity, 51% yield) of the title compound containing N-{4-[6-oxo-5-(quinolin-5-yl)-1,6-dihydropyridazin-3-yl] phenyl}-1,3-dihydro-2H-pyrrolo[3,4-c]pyridine-2-carboxamide as minor impurity.

LC-MS (Method 2): Rt=0.78 min, MS (ESpos): m/z=463 [M+H]⁺

¹H-NMR (400 MHz, DMSO-d₆) δ [ppm]: 3.29 (dd, 1H), 3.42 (dd, 1H), 4.72 (dd, 1H), 4.81 (br d, 4H), 7.43 (d, 1H), 7.52 (d, 1H), 7.56 (dd, 1H), 7.60-7.65 (m, 2H), 7.68-7.76 (m, 3H), 7.97 (d, 1H), 8.50 (d, 1H), 8.58-8.64 (m, 3H), 8.92 (dd, 1H), 11.20 (s, 1H).

### Intermediate 2-1

### Tert-Butyl {4-[-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}carbamate

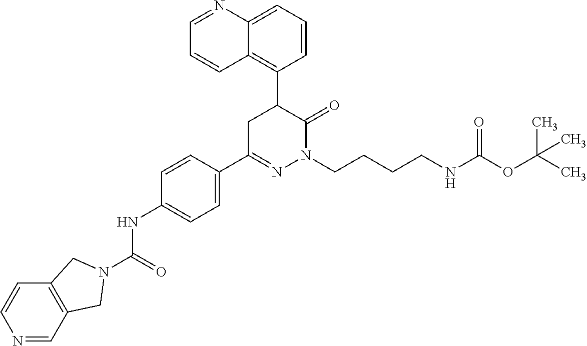 [SMILES: CC(C)(C)OC(=O)NCCCCN1N=C(c2ccc(NC(=O)N3Cc4ccncc4C3)cc2)CC(c2cccc3ncccc23)C1=O]

To a solution of N-{4-[6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 1, 25 mg, 54 μmol) in DMF (0.5 mL) and DMSO (0.2 mL) was added at −10° C. sodium hydride (4.5 mg, 60% in mineral oil, 114 μmol) under an argon atmosphere. The mixture stirred for 10 min at that temperature. Then tetra-n-butylammonium iodide (2.0 mg, 5.4 μmol) was added. After that tert-butyl (4-bromobutyl)carbamate (23.1 mg, 92 μmol) was added in 3 portions following every 10 min. After stirring for additional 30 min at −10° C. the mixture was was diluted with water, extracted with dichloromethane containing 10% ethanol. The organic extracts were filtered through a silicone filter and concentrated under reduced pressure and the crude product was purified by column chromatography (SiO₂, dichloromethane/ethanol gradient) followed by preparative HPLC to give 7.4 mg (90% purity, 20% yield) of the title compound and 4.1 mg of N-{4-[6-oxo-5-(quinolin-5-yl)-1,6-dihydropyridazin-3-yl]phenyl}-1,3-dihydro-2H-pyrrolo[3,4-c]pyridine-2-carboxamide and 2.1 mg of N-{4-[6-oxo-5-(quinolin-5-yl)-1,6-dihydropyridazin-3-yl]phenyl}-1,3-dihydro-2H-pyrrolo[3,4-c]pyridine-2-carboxamide (Example 4) as oxidative side products.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm, 150×20 mm. Eluent A: water+0.1% ammonia; Eluent B: acetonitrile; gradient: 0-14 min 1-30% B, 14-20 min 30% B, 20-25 min 30-50% B. rate 60 mL/min, temperature 25° C.

LC-MS (Method 2): Rt=1.05 min; MS (ESIpos): m/z=634 [M+H]⁺

¹H-NMR (400 MHz, DMSO-d₆) δ [ppm]: 1.37 (s, 9H), 1.39-1.49 (m, 2H), 1.64-1.74 (m, 2H), 2.93-3.00 (m, 2H), 3.26-3.32 (m, 1H), 3.31 (dd, 1H), 3.43 (dd, 1H), 3.77-3.90 (m, 2H), 4.76 (dd, 1H), 4.81 (br d, 4H), 6.84 (br t, 1H), 7.43 (br d, 1H), 7.45 (br d, 1H), 7.56 (dd, 1H), 7.64 (d, 2H), 7.70-7.75 (m, 3H), 7.96 (d, 1H), 8.50 (d, 1H), 8.58-8.64 (m, 3H), 8.92 (dd, 1H).

### Example 2

### N-{4-[1-(4-Aminobutyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide

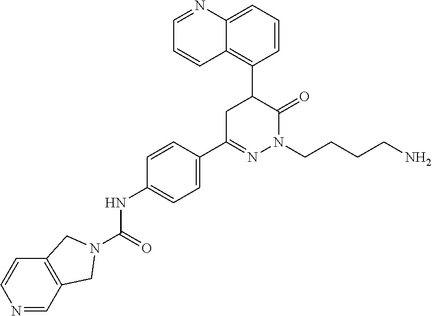 [SMILES: NCCCCN1N=C(c2ccc(NC(=O)N3Cc4ccncc4C3)cc2)CC(c2cccc3ncccc23)C1=O]

A mixture of tert-butyl {4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}carbamate and N-{4-[6-oxo-5-(quinolin-5-yl)-1,6-dihydropyridazin-3-yl]phenyl}-1,3-dihydro-2H-pyrrolo[3,4-c]pyridine-2-carboxamide (combined: 220 mg, 347 μmol), trifluoroacetic acid (530 μl, 6.9 mmol) and dichloromethane (3.4 mL) was stirred at r.t. for 2 h. Then the mixture was diluted with toluene, concentrated under reduced pressure and the crude product was purified by preparative HPLC to give 37.0 mg (90% purity, 18% yield) of the title compound and 88.8 mg (93% purity, 45.1% yield) of N-{4-[1-(4-aminobutyl)-6-oxo-5-(quinolin-5-yl)-1,6-dihydropyridazin-3-yl]phenyl}-1,3-dihydro-2H-pyrrolo[3,4-c]pyridine-2-carboxamide as a second product (Example 5).

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm. Eluent A: water+0.1% formic acid; Eluent B: acetonitrile; gradient: 0-7 min 1-7% B, 7-15 min 7-20% B. rate 150 mL/min, temperature 25° C.

LC-MS (Method 2): Rt=0.86 min; MS (ESpos): m/z=534 [M+H]⁺

¹H-NMR (400 MHz, DMSO-d₆) δ [ppm]: 1.57-1.64 (m, 2H), 1.72-1.81 (m, 2H), 2.81 (br t, 2H), 3.33 (dd, 1H), 3.46 (dd, 1H), 3.80-3.93 (m, 2H), 4.77 (dd, 1H), 4.81 (br d, 4H), 7.41-7.48 (m, 2H), 7.57 (dd, 1H), 7.65 (d, 2H), 7.68-7.76 (m, 3H), 7.97 (d, 1H), 8.36 (br s, 1H), 8.50 (d, 1H), 8.61 (d, 1H), 8.61 (s, 1H), 8.66 (s, 1H), 8.93 (d, 1H).

### Intermediate 3-1

Tert-Butyl {6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamate

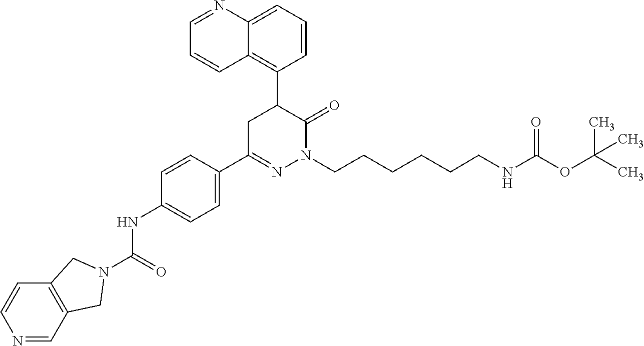 [SMILES: CC(C)(C)OC(=O)NCCCCCCN1N=C(c2ccc(NC(=O)N3Cc4ccncc4C3)cc2)CC(c2cccc3ncccc23)C1=O]

To a solution of N-{4-[6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 1, 220 mg, 478 μmol) in DMF (7.2 mL) and DMSO (4.4 mL) was added at 0° C. sodium hydride (47.8 mg, 60% in mineral oil, 1.19 mmol) under an argon atmosphere. The mixture stirred for 40 min at that temperature. Then tetra-n-butylammonium iodide (17.6 mg, 47.8 μmol) was added. After that tert-butyl (6-bromohexyl)carbamate (290 μL, 1.2 mmol) was added in 3 portions following every 4 min. After stirring for additional 2 h at 0° C. the mixture was diluted with saturated aqueous ammonium chloride solution and extracted with dichloromethane containing 10% ethanol. The organic extracts were filtered through a silicone filter, concentrated under reduced pressure, and the crude product was purified by column chromatography (SiO₂, dichloromethane/ethanol gradient) to give 231 mg (20% purity, 16% yield) of the title compound in a mixture with N-{4-[6-oxo-5-(quinolin-5-yl)-1,6-dihydropyridazin-3-yl]phenyl}-1,3-dihydro-2H-pyrrolo[3,4-c]pyridine-2-carboxamide as major impurity.

LC-MS (Method 2): Rt=1.17 min; MS (ESIpos): m/z=662 [M+H]⁺

¹H-NMR (400 MHz, DMSO-d₆): δ [ppm]=1.25-1.34 (m, 4H), 1.36 (s, 9H), 1.65-1.73 (m, 2H), 2.86-2.93 (m, 2H), 3.39-3.47 (m, 2H), 3.76-3.89 (m, 2H), 4.77 (m, 1H), 4.81 (br d, 4H), 6.78 (br t, 1H), 7.43 (d, 2H), 7.45 (d, 1H), 7.56 (dd, 1H), 7.64 (d, 2H), 7.69-7.75 (m, 3H), 7.96 (d, 1H), 8.49 (d, 1H), 8.58-8.65 (m, 2H), 8.92 (dd, 1H).

### Example 3

### N-{4-[1-(6-Aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide

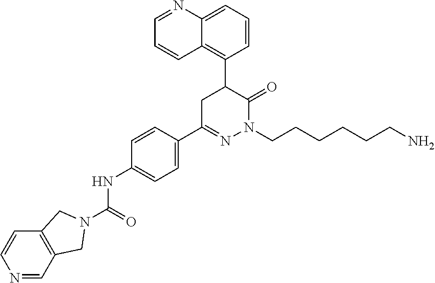 [SMILES: NCCCCCCN1N=C(c2ccc(NC(=O)N3Cc4ccncc4C3)cc2)CC(c2cccc3ncccc23)C1=O]

A mixture of tert-butyl {6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamate and N-{4-[6-oxo-5-(quinolin-5-yl)-1,6-dihydropyridazin-3-yl]phenyl}-1,3-dihydro-2H-pyrrolo[3,4-c]pyridine-2-carboxamide (combined: 231 mg, 349 μmol) and trifluoroacetic acid (540 μl, 7.0 mmol) in dichloromethane (3.4 mL) was stirred at r.t. for 2 h. Then the mixture was diluted with toluene, concentrated under reduced pressure and the crude product was purified by preparative HPLC to give 73.4 mg of the title compound (94% purity, 35% yield) and 120.3 mg (93% purity, 56% yield) of N-{4-[1-(4-aminobutyl)-6-oxo-5-(quinolin-5-yl)-1,6-dihydropyridazin-3-yl]phenyl}-1,3-dihydro-2H-pyrrolo[3,4-c]pyridine-2-carboxamide as a second product (Example 6) as their formic acid salts.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm. Eluent A: water+0.1% formic acid; Eluent B: acetonitrile; gradient: 0-7 min 1-7% B, 7-15 min 7-20% B rate 150 mL/min, temperature 25° C.

LC-MS (Method 1): Rt=0.63 min; MS (ESpos): m/z=562 [M+H]⁺

¹H-NMR (400 MHz, DMSO-d₆): δ [ppm]=1.32-1.40 (m, 4H), 1.48-1.56 (m, 2H), 1.66-1.76 (m, 2H), 2.76 (t, 2H), 3.33 (dd, 1H), 3.44 (dd, 1H), 3.63-3.91 (m, 2H), 4.79 (dd, 1H (, −4.81 (br d, 4H), 7.37-7.50 (m, 2H), 7.57 (dd, 1H), 7.61-7.78 (m, 5H), 7.97 (d, 2H), 8.22 (br s, 3H), 8.50 (d, 1H), 8.57-8.67 (m, 3H), 8.92 (dd, 1H).

### Example 4

### N-{4-[6-Oxo-5-(quinolin-5-yl)-1,6-dihydropyridazin-3-yl]phenyl}-1,3-dihydro-2H-pyrrolo[3,4-c]pyridine-2-carboxamide

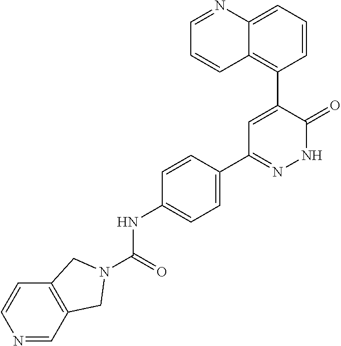 [SMILES: O=C(Nc1ccc(-c2cc(-c3cccc4ncccc34)c(=O)[nH]n2)cc1)N1Cc2ccncc2C1]

The title compound was isolated as a side product 2.1 mg (90% purity, 8% yield) from the preparation of tert-butyl {4-[-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}carbamate (see Intermediate 2-1).

LC-MS (Method 2): Rt=0.72 min; MS (ESpos): m/z=461 [M+H]⁺

¹H-NMR (400 MHz, DMSO-d₆) δ [ppm]: 4.83 (br d, 4H), 7.44 (d, 1H), 7.53 (dd, 1H), 7.65-7.75 (m, 3H), 7.84-7.93 (m, 3H), 8.11-8.15 (m, 2H), 8.16 (s, 1H), 8.50 (d, 1H), 8.62 (d, 2H), 8.95 (dd, 1H), 13.36 (s, 1H).

### Example 5

### N-{4-[1-(4-Aminobutyl)-6-oxo-5-(quinolin-5-yl)-1,6-dihydropyridazin-3-yl]phenyl}-1,3-dihydro-2H-pyrrolo[3,4-c]pyridine-2-carboxamide

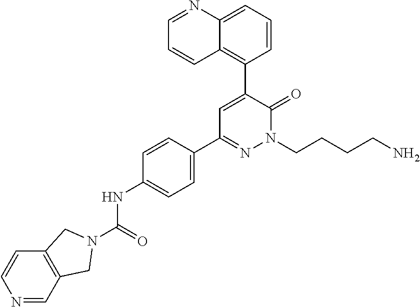 [SMILES: NCCCCn1nc(-c2ccc(NC(=O)N3Cc4ccncc4C3)cc2)cc(-c2cccc3ncccc23)c1=O]

The title compound was isolated as a second product 88.8 mg (93% purity, 45.1% yield) from the preparation of N-{4-[1-(4-aminobutyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 2).

LC-MS (Method 2): Rt=0.87 min; MS (ESpos): m/z=532 [M+H]⁺

¹H-NMR (400 MHz, DMSO-d₆): δ [ppm]=1.57-1.62 (m, 2H), 1.87-1.95 (m, 2H), 2.80 (br t, 2H), 4.25 (br t, 2H), 4.83 (br d, 4H), 7.44 (d, 1H), 7.53 (dd, 1H), 7.68 (dd, 1H), 7.73 (d, 2H), 7.86 (dd, 1H), 7.93 (d, 2H), 8.08-8.12 (m, 1H), 8.13 (d, 1H), 8.18 (s, 1H), 8.42 (br s, 1H), 8.51 (d, 1H), 8.62 (s, 1H), 8.68 (s, 1H), 8.95 (dd, 1H).

### Example 6

### N-{4-[1-(6-Aminohexyl)-6-oxo-5-(quinolin-5-yl)-1,6-dihydropyridazin-3-yl]phenyl}-1,3-dihydro-2H-pyrrolo[3,4-c]pyridine-2-carboxamide

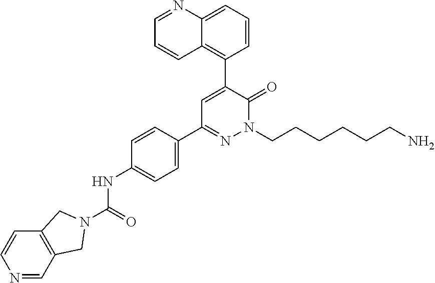 [SMILES: NCCCCCCn1nc(-c2ccc(NC(=O)N3Cc4ccncc4C3)cc2)cc(-c2cccc3ncccc23)c1=O]

The title compound was isolated as a second product 120.3 mg (93% purity, 56% yield) from the preparation of N-{4-[1-(6-aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 3) LC-MS (Method 1): Rt=0.62 min; MS (ESpos): m/z=561 [M+H]⁺

¹H-NMR (400 MHz, DMSO-d₆): δ [ppm]=1.33-1.43 (m, 4H), 1.47-1.57 (m, 2H), 1.81-1.88 (m, 2H), 2.72 (br t, 2H), 4.24 (br t, 2H), 4.83 (br d, 4H), 7.44 (d, 1H), 7.53 (dd, 1H), 7.68-7.78 (m, 3H), 7.86 (dd, 1H), −7.92 (d, 2H), 8.02-8.18 (m, 3H), 8.41 (br s, 1H), 8.51 (d, 1H), 8.62 (s, 1H), 8.68 (s, 1H), 8.95 (dd, 1H).

### Intermediate 7-1

### Ethyl 4-{4-[(tert-butoxycarbonyl)amino]phenyl}-2-(5-methyl-1,3,4-oxadiazol-2-yl)-4-oxobutanoate

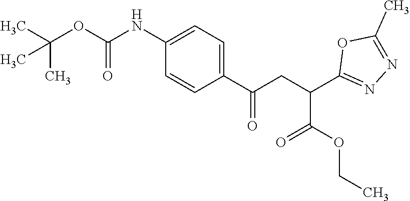 [SMILES: CCOC(=O)C(CC(=O)c1ccc(NC(=O)OC(C)(C)C)cc1)c1nnc(C)o1]

To ethyl-2-(5-methyl-1,3,4-oxadiazol-2-yl)acetate (Enamine EN300-159964, 948 mg, 5.57 mmol) in THE (45 mL) at 0° C. was added a solution of potassium bis(trimethylsilylamide) in toluene (13.4 mL, 0.5 M) and the mixture was stirred for 30 min at 0° C. A solution of [4-(bromoacetyl)phenyl]carbamate (CAS-885269-70-5, 2.5 g, 5.57 mmol) in THE (25 mL) was added at 0° C. and stirred for 1 additional hour. The mixture was diluted with saturated aqueous ammonium chloride solution, extracted with ethyl acetate, the combined organic phase was dried over sodium sulfate, and concentrated under reduced pressure. The crude product was purified by column chromatography (SiO₂, hexane/ethyl acetate gradient) followed by preparative HPLC to give 1004 mg (95% purity, 42% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-ODS-AQ 10 μm 280×51 mm. Eluent A: water+0.1% ammonia; Eluent B: acetonitrile; gradient: 0-20 min, 30-70% B, rate 250 mL/min, temperature 25° C.

LC-MS (Method 2): Rt=1.16 min; MS (ESIpos): m/z=404 [M+H]⁺

¹H-NMR (400 MHz, DMSO-d₆): δ [ppm]=1.17 (t, 3H), 1.49 (s, 9H), 3.71 (dd, 1H), 3.82 (dd, 1H), 4.11-4.18 (m, 2H), 4.56 (dd, 1H), 7.60 (d, 2H), 7.93 (d, 2H), 9.84 (s, 1H).

### Intermediate 7-2

### Tert-Butyl {4-[5-(5-methyl-1,3,4-oxadiazol-2-yl)-6-oxo-1,4,5,6-tetrahydropyridazin-3-yl]phenyl}carbamate

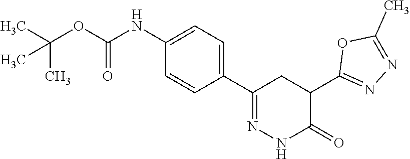 [SMILES: Cc1nnc(C2CC(c3ccc(NC(=O)OC(C)(C)C)cc3)=NNC2=O)o1]

Ethyl 4-{4-[(tert-butoxycarbonyl)amino]phenyl}-2-(5-methyl-1,3,4-oxadiazol-2-yl)-4-oxo butanoate (200 mg 496 μmol) was heated in dichloromethane (5 mL) in the presence of acetic acid (17 μL, 300 μmol) and hydrazine hydrate: (29 μL, 590 μmol) at 40° C. for 11 days in a closed vessel. The mixture was concentrated under reduced pressure and the crude product purified by column chromatography (SiO₂, hexane/ethyl acetate gradient) to yield 120 mg (95% purity, 62% yield) of the title compound.

LC-MS (Method 2): Rₜ=0.96 min; MS (ESIneg): m/z=370 [M−H]⁻

¹H-NMR (400 MHz, DMSO-d₆): δ [ppm]=1.48 (s, 9H), 2.48 (s, 3H), 3.25 (dd, 1H), 3.45 (dd, 1H), 4.44 (dd, 1H), 7.51 (d, 2H), 7.69 (d, 2H), 9.58 (s, 1H), 11.30 (s, 1H).

### Intermediate 7-3

### 6-(4-Aminophenyl)-4-(5-methyl-1,3,4-oxadiazol-2-yl)-4,5-dihydropyridazin-3(2H)-one

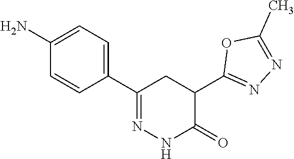 [SMILES: Cc1nnc(C2CC(c3ccc(N)cc3)=NNC2=O)o1]

A suspension of tert-butyl {4-[(5-(5-methyl-1,3,4-oxadiazol-2-yl)-6-oxo-1,4,5,6-tetrahydro pyridazin-3-yl]phenyl}carbamate (120 mg, 323 μmol) and trifluoroacetic acid (250 μl, 3.2 mmol) in dichloromethane (5.0 mL) was stirred at r.t. for 1 h. Then additional trifluoroacetic acid (250 μL, 3.2 mmol) was added to the yellow solution and stirring was continued for 1 h. The mixture was diluted with toluene, concentrated under reduced pressure and dried at high vacuum to yield 145 mg (60% purity, quant.) of the crude title compound, which was directly used in the next step.

LC-MS (Method 2): Rₜ=0.57 min; MS (ESIneg): m/z=270 [M−H]⁻.

¹H-NMR (400 MHz, DMSO-d₆) δ [ppm]: 2.52 (s, 3H), 3.22 (dd, 1H), 3.23, 3.40 (dd, 1H), 4.39 (dd, 1H), 5.12 (br s, 2H), 6.77 (br d, 2H), 7.58 (d, 2H), 11.19 (s, 1H).

### Intermediate 7-4

### 4-Nitrophenyl {4-[5-(5-methyl-1,3,4-oxadiazol-2-yl)-6-oxo-1,4,5,6-tetrahydropyridazin-3-yl]phenyl}carbamate

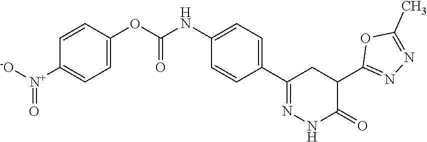

To 6-(4-aminophenyl)-4-(5-methyl-1,3,4-oxadiazol-2-yl)-4,5-dihydropyridazin-3(2H)-one (145 mg, 60% purity, 323 μmol) in THE (6.5 mL) was added at r.t. 4-nitrophenyl carbonochloridate (CAS-No. 7693-46-1, 129 mg, 641 μmol) and the mixture was heated to 60° C. After 1 h the mixture was concentrated under reduced pressure to give the raw title compound (quant.), which was directly used in the next step.

### Example 7

### N-{4-[5-(5-Methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide

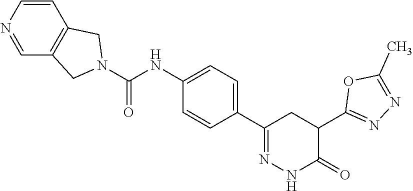 [SMILES: Cc1nnc(C2CC(c3ccc(NC(=O)N4Cc5ccncc5C4)cc3)=NNC2=O)o1]

2,3-Dihydro-1H-pyrrolo[3,4-c]pyridine dihydrochloride (CAS-No. 6000-50-6, 92.2 mg, 478 μmol) was added to a suspension of raw 4-nitrophenyl {4-[5-(5-methyl-1,3,4-oxadiazol-2-yl)-6-oxo-1,4,5,6-tetrahydropyridazin-3-yl]phenyl}carbamate (see Intermediate 7-4, 323 μmol) in dichloromethane (6.6 mL) and N,N-diisopropylethylamine (280 μl, 1.6 mmol). After stirring at r. t. for 3 h the mixture was concentrated under reduced pressure and purified by preparative HPLC to give 52.0 mg (95% purity, 37% yield) of the title compound HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18 10 μm 125×30 mm. Eluent A: water+0.1% ammonia; Eluent B: acetonitrile; gradient: 0-6 min 10-50% B, 6-8 min 50-100% B, rate 150 mL/min, temperature 25° C.

LC-MS (Method 2): Rt=0.67 min; MS (ESipos): m/z=418 [M+H]⁺.

¹H-NMR (400 MHz, DMSO-d₆): δ [ppm]: 2.51 (s, 3H), 3.28 (dd, 1H), 3.47 (dd, 1H), 4.45 (dd, 1H), 4.82 (br d, 4H), 7.44 (d, 1H), 7.66 (d, 2H), 7.72 (d, 2H), 8.51 (d, 1H), 8.62 (s, 1H), 8.64 (s, 1H) 11.30 (s, 1H).

### Intermediate 8-1

### Tert-Butyl {4-[3-{4-[(1,3-dihydro-2H-pyrrolo[3,4-c]pyridin-2-ylcarbonyl)amino]phenyl}-5-(5-methyl-1,3,4-oxadiazol-2-yl)-6-oxopyridazin-1(6H)-yl]butyl}carbamate

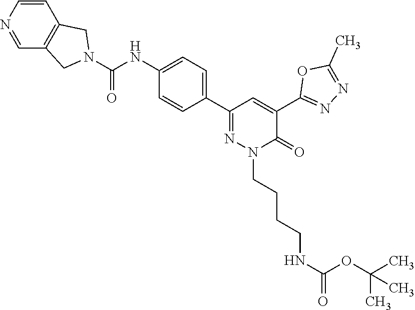 [SMILES: Cc1nnc(-c2cc(-c3ccc(NC(=O)N4Cc5ccncc5C4)cc3)nn(CCCCNC(=O)OC(C)(C)C)c2=O)o1]

To a solution of N-{4-[5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide (see Example 7, 113 mg, 270 μmol) in DMF (1.8 mL) and DMSO (1.8 mL) was added at 0° C. sodium hydride (9.70 mg, 60% in mineral oil 404 μmol) under an argon atmosphere and the mixture was stirred for 20 min at that temperature. Then tetra-n-butylammonium iodide (9.95 mg, 27.0 μmol) and tert-butyl (4-bromobutyl)carbamate (109 mg, 431 μmol) was added in 5 portions all 10 min. After stirring for one additional hour at 0° C. the mixture was diluted with saturated aqueous ammonium chloride solution and concentrated under reduced pressure The residue was suspended in DMSO, filtrated and the filtrate was purified by preparative HPLC to give 65.6 mg (95% purity, 39.4% yield) of the title compound together with 23.9 mg (95% purity, 21.3% yield) of N-{4-[5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (Example 9) HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-6 min 10-50% B, 6-8 min 50-100% B, rate 150 mL/min, temperature 25° C.

LC-MS (Method 2): Rt=0.99 min; MS (ESIneg): m/z=585 [M−H]⁻

¹H-NMR (400 MHz, DMSO-d₆) δ [ppm]: 1.36 (s, 9H), 1.41-1.48 (m, 2H), 1.75-1.85 (m, 2H), 2.63 (s, 3H), 2.97 (q, 2H), 4.23 (br t, 2H), 4.84 (br d, 4H), 6.85 (br t, 1H), 7.45 (d, 1H), 7.69-7.78 (m, 2H), 7.85-7.92 (m, 2H), 8.51 (d, 1H), 8.52 (s, 1H), 8.62 (s, 1H), 8.68 (s, 1H).

### Example 8

### N-{4-[1-(4-Aminobutyl)-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide

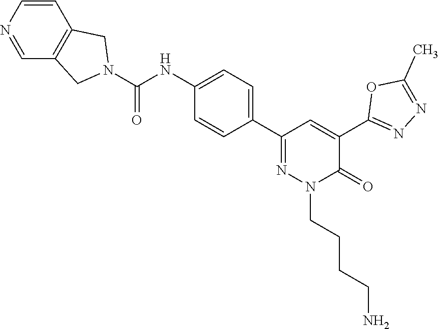 [SMILES: Cc1nnc(-c2cc(-c3ccc(NC(=O)N4Cc5ccncc5C4)cc3)nn(CCCCN)c2=O)o1]

Tert-butyl {4-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]butyl}carbamate (96.7 mg, 165 μmol) was stirred in dichloromethan (2 mL) together with trifluoroacetic acid (200 μl, 2.6 mmol). After 1 h the mixture was concentrated under reduced pressure and purified by preparative HPLC to give 80.4 mg (95% purity, 95% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% formic acid; Eluent B: acetonitrile; gradient: 0-6 min 10-50% B, 6-8 min 50-100% B, rate 150 mL/min, temperature 25° C.

LC-MS (Method 2): Rₜ=0.75 min: MS (ESIpos): m/z=487 [M+H]⁺

¹H-NMR (400 MHz, DMSO-d6): δ [ppm]=1.55-1.67 (m, 2H), 1.83-1.96 (m, 2H), 2.63 (s, 3H), 2.85 (t, 2H), 4.27 (t, 2H), 4.84 (br d, 4H), 7.45 (d, 1H), 7.76 (d, 2H), 7.90 (d, 2H), 8.16 (s, 2H), 8.51 (d, 1H), 8.54 (s, 1H), 8.62 (s, 1H), 8.70 (s, 1H).

### Example 9

### N-{4-[5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide

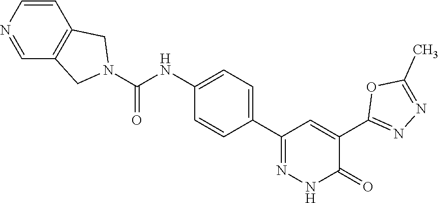 [SMILES: Cc1nnc(-c2cc(-c3ccc(NC(=O)N4Cc5ccncc5C4)cc3)n[nH]c2=O)o1]

The title compound was isolated as a second product 23.9 mg (95% purity, 21.3% yield) from the preparation of tert-butyl {4-[3-{4-[(1,3-dihydro-2H-pyrrolo[3,4-c]pyridin-2-ylcarbonyl) amino]phenyl}-5-(5-methyl-1,3,4-oxadiazol-2-yl)-6-oxopyridazin-1(6H)-yl]butyl}carbamate (see Intermediate 8-1).

LC-MS (Method 2): Rt=0.55 min; MS (ESIpos): m/z=416 [M+H]⁺

¹H-NMR (400 MHz, DMSO-d6) δ [ppm]: 2.63 (s, 3H), 4.83 (br d, 4H), 7.45 (d, 1H), 7.73 (d, 2H), 7.85 (d, 2H), 8.51 (s, 2H), 8.62 (s, 1H), 8.67 (s, 1H), 13.70 (s, 1H).

### Intermediate 10-1

### Tert-Butyl {6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}carbamate

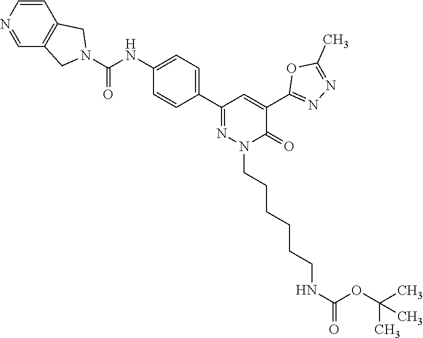 [SMILES: Cc1nnc(-c2cc(-c3ccc(NC(=O)N4Cc5ccncc5C4)cc3)nn(CCCCCCNC(=O)OC(C)(C)C)c2=O)o1]

To a suspension of N-{4-[5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide (see Example 7, 74.0 mg, 177 μmol) in DMF (1.7 mL) and DMSO (1.7 mL) was added at r.t. sodium hydride (14.9 mg, 60% in mineral oil, 372 μmol) under an argon atmosphere. The mixture was stirred for 20 min at r. t. Then tetra-n-butylammonium iodide (6.55 mg, 17.7 μmol) and tert-butyl (6-bromohexyl)carbamate (89 μl, 380 μmol) was added. After stirring for 4 h at r,t. the mixture was diluted with saturated aqueous ammonium chloride solution and concentrated under reduced pressure The residue was suspended in an acetonitrile DMSO mixture, filtrated and the concentrated filtrate was purified by preparative HPLC to give 115 mg (60% purity, 63% yield) of the title compound in a mixture with tert-butyl {6-[3-{4-[(1,3-dihydro-2H-pyrrolo[3,4-c]pyridine-2-carbonyl)amino] phenyl}-5-(5-methyl-1,3,4-oxadiazol-2-yl)-6-oxo-5,6-dihydropyridazin-1(4H)-yl]hexyl} carbamate as minor second product.

HPLC: Instrument: Labomatic HD-5000, pump head HDK-280, gradient module NDB-1000, fraction collector Labomatic Labocol Vario 4000, Knauer UV detector Azura UVD 2.1S, Prepcon 5 software. Column: Chromatorex C18 10 μm 125×30 mm; Eluent A: water+0.1% ammonia; Eluent B: acetonitrile; gradient: 0-7 min 30-70% B, rate 150 mL/min, temperature 25° C.

LC-MS (Method 2): Rt=1.06 min; MS (ESIpos): m/z=615 [M+H]⁺

¹H-NMR (400 MHz, DMSO-d₆) δ [ppm]: 1.18-1.35 (m, 6H), 1.36 (s, 9H), 1.76-1.86 (m, 2H), 2.63 (s, 3H), 2.85-2.92 (m, 2H), 4.22 (br t, 2H), 4.84 (br d, 4H), 6.77 (br t, 1H), 7.45 (br s, 1H), 7.74 (d, 2H), 7.88 (d, 2H), 8.49-8.53 (m, 2H), 8.62 (s, 1H), 8.62 (s, 1H), 8.68 (s, 1H).

### Example 10

### N-{4-[1-(6-Aminohexyl)-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide

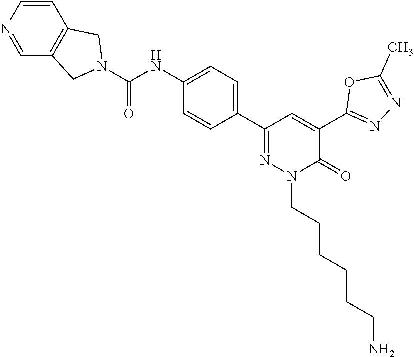 [SMILES: Cc1nnc(-c2cc(-c3ccc(NC(=O)N4Cc5ccncc5C4)cc3)nn(CCCCCCN)c2=O)o1]

A mixture of tert-butyl {6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}carbamate, tert-butyl {6-[3-{4-[(1,3-dihydro-2H-pyrrolo[3,4-c]pyridine-2-carbonyl)amino]phenyl}-5-(5-methyl-1,3,4-oxadiazol-2-yl)-6-oxo-5,6-dihydropyridazin-1(4H)-yl]hexyl}carbamate. (combined 115 mg, 187 μmol), and trifluoro acetic acid (230 μL, 3.0 mmol) in dichloromethane (2.3 mL) was stirred at r.t. for 3 h. The mixture was concentrated under reduced pressure and purified by preparative HPLC to give 40.4 mg (95% purity, 39% yield) of the title compound together with 11.5 mg (80% purity, 9.5% yield) of N-{4-[1-(6-aminohexyl)-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide (Example 11).

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% formic acid; Eluent B: acetonitrile; gradient: 0-6 min 5-30% B, rate 150 mL/min, temperature 25° C.

LC-MS (Method 2): Rt=0.57 min; MS (ESIpos): m/z=515 [M+H]⁺

¹H-NMR (400 MHz, DMSO-d₆) δ [ppm]: 1.33-1.41 (m, 4H), 1.46-1.56 (m, 2H), 1.83 (br t, 2H), 2.63 (s, 3H), 2.74 (t, 2H), 4.24 (t, 2H), 4.84 (br d, 4H), 7.45 (d, 1H), 7.67-7.82 (m, 2H), 7.82-7.97 (m, 2H), 8.37 (br s, 1H), 8.51 (d, 1H), 8.52 (s, 1H), 8.62 (s, 1H), 8.71 (s, 1H).

### Example 11

### N-{4-[1-(6-Aminohexyl)-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide

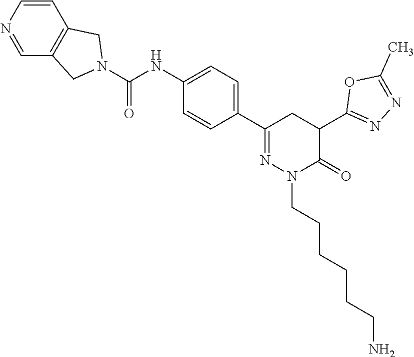 [SMILES: Cc1nnc(C2CC(c3ccc(NC(=O)N4Cc5ccncc5C4)cc3)=NN(CCCCCCN)C2=O)o1]

The title compound was isolated as a second product 11.5 mg (80% purity, 9.5% yield) from the preparation of N-{4-[1-(6-aminohexyl)-5-(5-methyl-1,3,4-oxadiazol-2-yl)-6-oxo-1,6-dihydro pyridazin-3-yl]phenyl}-1,3-dihydro-2H-pyrrolo[3,4-c]pyridine-2-carboxamide (see Example 10).

LC-MS (Method 2): Rt=0.88 min; MS (ESIneg): m/z=515 [M−H]⁻

¹H-NMR (400 MHz, DMSO-d6): δ [ppm]: 1.23-1.35 (m, 4H), 1.51 (br quin, 2H), 1.64 (br quin, 2H), 2.51 (s, 3H), 2.72 (br t, 1H), 2.69-2.75 (m, 1H), 3.32 (dd, 1H), 3.31-3.37 (m, 1H), 3.48 (dd, 1H), 3.76 (br t, 2H), 4.50 (dd, 1H), 4.83 (br d, 4H), 7.44 (br d, 1H), 7.69 (d, 2H), 7.75 (d, 2H), 8.41 (br s, 2H), 8.51 (br d, 1H), 8.62 (s, 1H), 8.73 (s, 1H).

### Intermediate 12-1

### 2,2,2-Trifluoro-1-(quinolin-5-yl)ethan-1-one

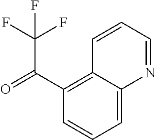 [SMILES: O=C(c1cccc2ncccc12)C(F)(F)F]

5-Bromoquinoline (CAS 4964-71-0, 6.15 g, 29.6 mmol) was added portion-wise to a solution of n-butyl lithium (1.6M in Hexan, 37 mL) in THE (150 mL) and diethylether (150 mL) at −70° C. After stirring for 30 min a −70° C. cold solution of N,N-diethyl-2,2,2-trifluoroacetamide (4.2 mL, 30 mmol) in THE (50 mL) was added and the mixture was stirred for 1 h at −78° C.

The reaction was quenched with saturated aqueous ammonium chloride solution, warmed to r.t. and extracted with ethyl acetate. The organic extracts were washed with brine, concentrated, and the crude product was purified by column chromatography (SiO₂, hexane/ethyl acetated gradient) to yield 3.77 g (95% purity, 50% yield) of the partially hydrated title compound.

LC-MS (Method 2): Rt=0.82 min, MS (ESIpos): m/z=226 [M+H]⁺, m/z=244 [M+H₃O]⁺.

¹H-NMR (400 MHz, DMSO-d6) δ [ppm]: 2.518 (3.11), 2.523 (2.35), 7.524 (9.81), 7.532 (4.39), 7.535 (10.13), 7.545 (12.52), 7.555 (11.68), 7.598 (4.22), 7.601 (4.27), 7.615 (5.69), 7.618 (9.65), 7.622 (4.89), 7.636 (6.13), 7.639 (5.86), 7.750 (5.32), 7.754 (5.84), 7.757 (4.89), 7.767 (9.70), 7.771 (12.14), 7.775 (7.12), 7.778 (5.28), 7.789 (12.57), 7.791 (7.92), 7.810 (3.80), 7.974 (5.38), 7.977 (7.51), 7.981 (6.89), 7.993 (5.74), 7.996 (7.76), 7.998 (7.36), 8.002 (6.50), 8.014 (16.00), 8.034 (8.19), 8.081 (8.41), 8.099 (3.49), 8.306 (2.01), 8.309 (2.38), 8.314 (2.09), 8.319 (1.30), 8.325 (1.84), 8.328 (2.09), 8.333 (1.77), 8.362 (5.62), 8.365 (5.72), 8.367 (5.30), 8.381 (5.16), 8.383 (5.38), 8.386 (5.42), 8.435 (4.93), 8.456 (4.32), 8.879 (3.46), 8.883 (3.75), 8.889 (3.56), 8.893 (3.56), 8.902 (8.05), 8.907 (8.05), 8.913 (8.29), 8.917 (7.90), 9.008 (2.82), 9.010 (3.24), 9.012 (3.71), 9.014 (3.12), 9.029 (2.68), 9.032 (3.16), 9.034 (3.48), 9.036 (3.19), 9.056 (4.88), 9.060 (4.14), 9.066 (4.86), 9.070 (3.83), 9.169 (2.06), 9.192 (1.97).

### Intermediate 12-2

### (2Z)-4,4,4-trifluoro-1-(4-nitrophenyl)-3-(quinolin-5-yl)but-2-en-1-one

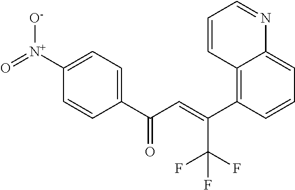 [SMILES: O=C(/C=C(/c1cccc2ncccc12)C(F)(F)F)c1ccc([N+](=O)[O-])cc1]

To a mixture of 1-(4-nitrophenyl)ethan-1-one (CAS 100-19-6, 240 mg, 1.45 mmol) and 2,2,2-trifluoro-1-(quinolin-5-yl)ethan-1-one (600 mg, 60% purity, 1.60 mmol) in THE (3.5 mL) was added 1,8-diazabicyclo(5.4.0)undec-7-en (220 μl, 1.5 mmol). The mixture was stirred for 16 h at r.t., then heated to 50 C for 8 h and cooled to r.t. over one day while stirring. The mixture was concentrated under reduced pressure and the crude product was purified by column chromatography (SiO₂, hexane/ethyl acetate gradient) to give 148 mg (60% purity, 16% yield) of the title compound.

LC-MS (Method 2): Rt=1.24 min; MS (ESIpos): m/z=373 [M+H]⁺

¹H-NMR (400 MHz, DMSO-d₆) δ [ppm]: 7.53 (d, 1H), 7.54 (dd, 1H), 7.74 (dd, 1H), 8.06 (d, 1H), 8.07-8.10 (m, 2H), 8.19-8.20 (m, 1H), 8.21-8.27 (m, 3H), 8.92 (dd, 1H).

### Intermediate 12-3

### (2R)-4-(4-Nitrophenyl)-4-oxo-2-(quinolin-5-yl)-2-(trifluoromethyl)butanenitrile

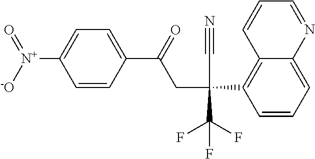

(2Z)-4,4,4-Trifluoro-1-(4-nitrophenyl)-3-(quinolin-5-yl)but-2-en-1-one (590 mg, 1.58 mmol) was solved in THE (66 mL) and degassed with nitrogen. Then potassium carbonate (657 mg, 4.75 mmol), (9S)-1-{[3,5-bis(trifluoromethyl)phenyl]methyl}-6′,9-dimethoxycinchonan-1-ium bromide (prepared according to Eur. J. Org. Chem. 2013, 5398-5413, 205 mg, 317 μmol) and 2-hydroxy-2-methylpropanenitrile (440 μl, 4.8 mmol) were added at 0° C. The mixture was warmed to r. t and stirred for one day. After concentration of the mixture under reduced pressure the solvent was exchanged to ethyl acetate. The organic phase was washed with saturated aqueous ammonium chloride solution and brine. The organic phase was concentrated under reduced pressure and the crude product was purified by column chromatography (SiO₂, hexane/ethyl acetate gradient 0%-40%) to give 370 mg (90% purity, 53% yield) of the title compound.

LC-MS (Method 2): Rt=1.21 min; MS (ESIpos): m/z=400 [M+H]⁺

¹H-NMR (400 MHz, DMSO-d₆): δ [ppm]=4.81 (d, 1H), 5.50 (d, 1H), 7.73-7.81 (m, 2H), 7.96 (br d, 1H), 8.18 (d, 1H), 8.29-8.33 (m, 2H), 8.36-8.41 (m, 2H), 9.00-9.05 (m, 1H), 9.16 (br d, 1H).

### Intermediate 12-4

### (2R)-4-Hydrazinylidene-4-(4-nitrophenyl)-2-(quinolin-5-yl)-2-(trifluoromethyl)butanamide

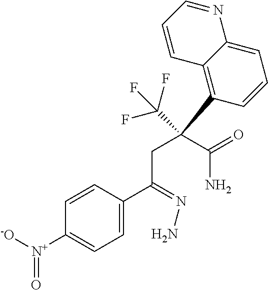

To (2R)-4-(4-nitrophenyl)-4-oxo-2-(quinolin-5-yl)-2-(trifluoromethyl)butanenitrile (370 mg, 927 μmol) in ethanol (2.2 mL) was added at 0 C hydrazine hydrate (1.6 mL, 32 mmol) and the mixture was heated to 50 C for 15 min. After that the mixture was cooled to r.t. and water (10 mL) was added. The formed precipitate was isolated by concentration of the mixture under reduced pressure to give the raw title compound 490 mg (57% purity, 70% yield) which was directly used in the next step.

LC-MS (Method 2): Rt=0.95 min; MS (ESIneg): m/z=430 [M−H]⁻

### Intermediate 12-5

### (4R)-6-(4-Nitrophenyl)-4-(quinolin-5-yl)-4-(trifluoromethyl)-4,5-dihydropyridazin-3(2H)-one

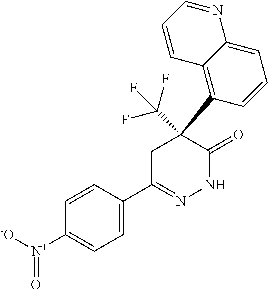

To (2R)-4-hydrazinylidene-4-(4-nitrophenyl)-2-(quinolin-5-yl)-2-(trifluoromethyl)butanamide (480 mg, 1.11 mmol) in ethanol (12 mL) and water was added acetic acid (320 μl, 5.6 mmol) and the mixture was stirred for 10 h at 90° C. After cooling to r.t. to the mixture was concentrated under reduced pressure and the crude product was purified by preparative HPLC to give 40.0 mg (95% purity, 8% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm; Eluent A: water+0.1% ammonia; Eluent B: acetonitrile; gradient: 0-7 min 30-70% B, rate 150 mL/min, temperature 25° C.

LC-MS (Method 2): Rt=1.08 min; MS (ESipos): m/z=415 [M+H]⁺

¹H-NMR (400 MHz, DMSO-d₆): δ [ppm]=3.81 (d, 1H), 4.60 (d, 1H), 7.53-7.70 (m, 2H), 7.74 (d, 1H), 8.07 (d, 1H), 8.10 (d, 2H), 8.25 (d, 2H), 8.93 (d, 1H), 9.03 (d, 1H), 12.06 (br s, 1H).

### Intermediate 12-6

### (4R)-6-(4-Aminophenyl)-4-(quinolin-5-yl)-4-(trifluoromethyl)-4,5-dihydropyridazin-3(2H)-one

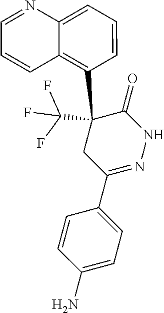 [SMILES: Nc1ccc(C2=NNC(=O)[C@@](c3cccc4ncccc34)(C(F)(F)F)C2)cc1]

To (4R)-6-(4-nitrophenyl)-4-(quinolin-5-yl)-4-(trifluoromethyl)-4,5-dihydropyridazin-3(2H)-one (40.0 mg, 96.5 μmol) in ethyl acetate (5.0 mL) was added palladium on charcoal (20 mg, 10%). The mixture was stirred under a hydrogen atmosphere for 2 h at r. t. The hydrogen atmosphere was exchanged to argon and the mixture was filtered through a short path of celite, which was thoroughly washed with ethyl acetate afterwards. The combined filtrates were concentrated under reduced pressure to yield 29.3 mg (70% purity, 55% yield) of the title compound.

LC-MS (Method 2): Rt=0.91 min; MS (ESIpos): m/z=385 [M+H]⁺

¹H-NMR (400 MHz, CDCl₃): δ [ppm]=3.51 (d, 1H), 3.98 (br s, 2H), 4.04 (d, 1H), 6.66-6.72 (m, 2H), 7.43 (dd, 1H), 7.52-7.63 (m, 3H), 8.13 (d, 1H), 8.61 (s, 1H), 8.91 (dd, 1H), 9.00 (d, 1H).

### Intermediate 12-7

### 4-Nitrophenyl {4-[(5R)-6-oxo-5-(quinolin-5-yl)-5-(trifluoromethyl)-1,4,5,6-tetrahydropyridazin-3-yl]phenyl}carbamate

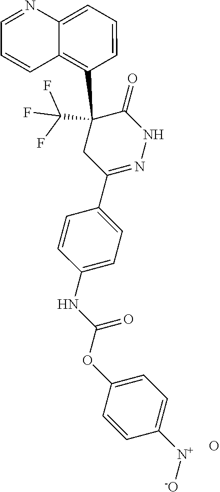

To (4R)-6-(4-aminophenyl)-4-(quinolin-5-yl)-4-(trifluoromethyl)-4,5-dihydropyridazin-3(2H)-one (29.3 mg, 70% purity, 76.2 μmol) in THE (6.5 mL) was added at r.t. 4-nitrophenyl carbonochloridate (CAS-No. 7693-46-1, 30.7 mg, 152 μmol) and the mixture was heated to 60° C. After 1.5 h the mixture was concentrated under reduced pressure to give the raw title compound (quant.), which was directly used in the next step.

LC-MS (Method 1): Rt=1.16 min; MS (ESIpos): m/z=550 [M+H]⁺

### Example 12

### N-{4-[(5R)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide

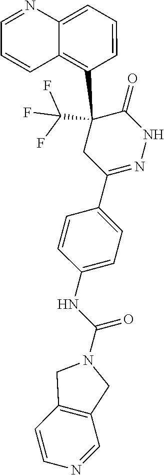 [SMILES: O=C(Nc1ccc(C2=NNC(=O)[C@@](c3cccc4ncccc34)(C(F)(F)F)C2)cc1)N1Cc2ccncc2C1]

2,3-Dihydro-1H-pyrrolo[3,4-c]pyridine dihydrochloride (CAS-No. 6000-50-6, 22.1 mg, 115 μmol) was added to a suspension of raw 4-nitrophenyl{4-[(5R)-6-oxo-5-(quinolin-5-yl)-5-(trifluoromethyl)-1,4,5,6-tetrahydropyridazin-3-yl]phenyl}carbamate (see Intermediate 12-7, 42.0 mg, 76.2 μmol) in dichloromethane (1.6 mL) and N,N-diisopropylethylamine (67 μl, 380 μmol). After stirring at r. t. for 3 h and storage at −20° C. for 16 h the mixture was concentrated under reduced pressure and purified by preparative HPLC to give 18.6 mg (90% purity, 41% yield of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. column: Chromatorex C18 10 μm 125×30 mm. Eluent A: water+0.1% ammonia; Eluent B: acetonitrile; gradient: 0-6 min 15-55% B, 6-8 min 55-100% B, rate 150 mL/min, temperature 25° C.

LC-MS (Method 2): Rt=0.93 min; MS (ESpos): m/z=531 [M+H]⁺

¹H-NMR (400 MHz, DMSO-d6) δ [ppm]: 3.60 (d, 1H), 4.47 (br d, 1H), 4.82 (br d, 4H), 7.44 (d, 1H), 7.59 (dd, 1H), 7.62-7.69 (m, 3H), 7.73-7.83 (m, 3H), 8.05 (d, 1H), 8.51 (d, 1H), 8.63 (d, 2H), 8.92 (dd, 1H), 9.11 (br d, 1H), 11.66 (s, 1H).

Chiral LC: Instrument: Agilent HPLC 1260; column: Chiralpak IB 3μ 100×4.6 mm; eluent A: water+0.1 Vol-% phosphoric acid (85%), eluent B: acetonitril; gradient: 0-7 min 20-90% B; flow 1.4 mL/min; temperature: 25° C.; DAD @ 325 nm: Rt=2.63 min, ee=99%.

### Intermediate 13-1

### Tert-Butyl {4-[(5R)-3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]butyl}carbamate

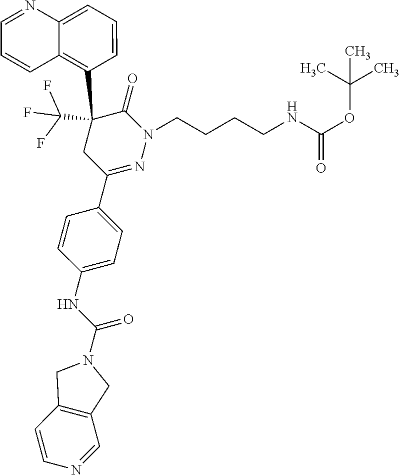 [SMILES: CC(C)(C)OC(=O)NCCCCN1N=C(c2ccc(NC(=O)N3Cc4ccncc4C3)cc2)C[C@](c2cccc3ncccc23)(C(F)(F)F)C1=O]

To a solution of N-{4-[(5R)-6-oxo-5-(quinolin-5-yl)-5-(trifluoromethyl)-1,4,5,6-tetrahydro pyridazin-3-yl]phenyl}-1,3-dihydro-2H-pyrrolo[3,4-c]pyridine-2-carboxamide (see Example 12, 13.0 mg, 24.5 μmol) in DMF (0.5 mL) was added at 0° C. sodium hydride (2.06 mg, 60% on mineral oil, 55.5 μmol). The mixture was stirred for 5 min at that temperature. Then tetra-n-butylammonium iodide (910 μg, 2.5 μmol) was added. After that tert-butyl (4-bromobutyl)carbamate (10.5 mg, 41.7 μmol) was added. After stirring for 4 h at r.t. the mixture was purified by preparative HPLC to give 9.6 mg (95% purity, 57% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-7 min 30-70% B, rate 150 mL/min, temperature 25° C.

LC-MS (Method 2): Rt=1.19 min; MS (ESIpos): m/z=702 [M+H]⁺

¹H-NMR (400 MHz, DMSO-d6): δ [ppm]=1.13-1.29 (m, 2H), 1.35 (s, 9H), 1.41-1.57 (m, 2H), 2.83 (q, 2H), 3.60 (d, 1H), 3.66-3.82 (m, 3H), 4.53 (br d, 1H), 4.83 (br d, 4H), 6.73 (br t, 1H), 7.45 (d, 1H), 7.55-7.69 (m, 4H), 7.74-7.86 (m, 3H), 8.04 (d, 1H), 8.51 (d, 1H), 8.62 (s, 1H), 8.68 (s, 1H), 8.90 (dd, 1H), 9.02 (d, 1H).

### Example 13

### N-{4-[(5R)-1-(4-aminobutyl)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide

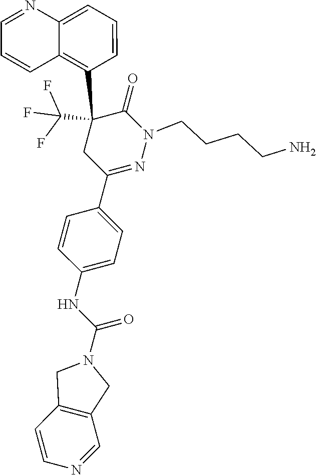 [SMILES: NCCCCN1N=C(c2ccc(NC(=O)N3Cc4ccncc4C3)cc2)C[C@](c2cccc3ncccc23)(C(F)(F)F)C1=O]

Tert-butyl {4-[(5R)-3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]butyl}carbamate (9.60 mg, 13.7 μmol) was stirred in dichloromethan (800 μL) together with trifluoroacetic acid (105 μl, 1.35 mmol), which was added in three portions over 3 hours. The mixture was concentrated under reduced pressure and purified by preparative HPLC to give 8.7 mg (90% purity, 95% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm; Eluent A: water+0.1% formic acid; Eluent B: acetonitrile; gradient: 0-14 min 15-55% B, 14-17 min 55-100% B, rate 60 mL/min, temperature 25° C.

LC-MS (Method 2): Rt=1.00 min; MS (ESIpos): m/z=602 [M+H]⁺

¹H-NMR (400 MHz, METHANOL-d₄): δ [ppm]=1.48-1.61 (m, 2H), 1.62-1.77 (m, 2H), 2.79-2.91 (m, 2H), 3.62 (d, 1H), 3.79-3.96 (m, 2H), 4.48 (d, 1H), 4.92 (br d, 4H), 7.49 (d, 1H), 7.54-7.63 (m, 4H), 7.74-7.81 (m, 3H), 8.03 (s, 1H), 8.07 (s, 1H), 8.10-8.25 (m, 1H), 8.49 (br d, 1H), 8.58 (br s, 1H), 8.85 (dd, 1H), 9.18 (d, 1H).

### Intermediate 14-1

### 4-(4-Nitrophenyl)-4-oxo-2-(quinolin-5-yl)-2-(trifluoromethyl)butanenitrile

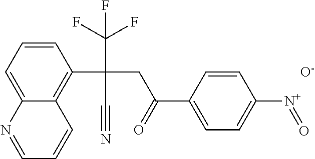

(2Z)-4,4,4-Trifluoro-1-(4-nitrophenyl)-3-(quinolin-5-yl)but-2-en-1-one (see Intermediate 12-2, 800 mg, 2.15 mmol) was solved in THE (66 mL). Then potassium carbonate (891 mg, 6.45 mmol), (8α,9R)-1-{[3,5-bis(trifluoromethyl)phenyl]methyl}-6′,9-dimethoxycinchonan-1-ium bromide (prepared in analogy to Eur. J. Org. Chem. 2013, 5398-5413, 347 mg, 80% purity, 430 μmol) and 2-hydroxy-2-methylpropanenitrile (590 μl, 6.4 mmol) were added at r.t. The mixture was warmed to 40° C. and stirred for one day. After concentration of the mixture under reduced pressure the solvent was exchanged to ethyl acetate. The organic phase was washed with saturated aqueous ammonium chloride solution and brine. The organic phase was concentrated under reduced pressure and the crude product was purified by column chromatography (SiO₂, hexane/ethyl acetate gradient 0%-50%) to give 593 mg (90% purity, 62% yield) of the title compound.

LC-MS (Method 2): Rt=1.24 min; MS (ESIneg): m/z=398 [M−H]⁻

¹H-NMR (400 MHz, DMSO-d₆): δ [ppm]=4.81 (d, 1H), 5.50 (d, 1H), 7.73-7.81 (m, 2H), 7.96 (br d, 1H), 8.18 (d, 1H), 8.29-8.33 (m, 2H), 8.36-8.41 (m, 2H), 9.00-9.05 (m, 1H), 9.16 (br d, 1H).

### Intermediate 14-2

### 4-Hydrazinylidene-4-(4-nitrophenyl)-2-(quinolin-5-yl)-2-(trifluoromethyl)butanamide

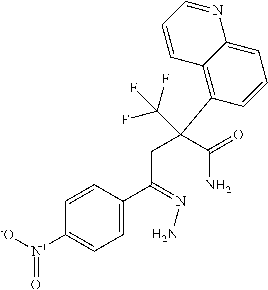

To 4-(4-nitrophenyl)-4-oxo-2-(quinolin-5-yl)-2-(trifluoromethyl)butanenitrile (780 mg, 1.95 mmol) in ethanol (9.1 mL) was added hydrazine hydrate (3.3 mL, 68 mmol) at 0 C and the mixture was heated to 50 C for 3 h. The mixture was concentrated under reduced pressure to give 843 mg (quant.) of the raw title compound which was directly used in the next step.

LC-MS (Method 2): Rt=0.93 min; MS (ESIpos): m/z=432 [M+H]⁺

### Intermediate 14-3

### 6-(4-Nitrophenyl)-4-(quinolin-5-yl)-4-(trifluoromethyl)-4,5-dihydropyridazin-3(2H)-one

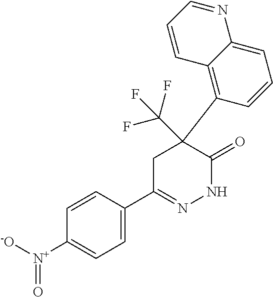

To 4-hydrazinylidene-4-(4-nitrophenyl)-2-(quinolin-5-yl)-2-(trifluoromethyl)butanamide (raw intermediate 14-2, 843 mg, 1.95 mmol) in ethanol (22 mL) and water (22 mL) was added acetic acid (560 μl, 9.8 mmol) and the mixture was stirred for 2 days at 90° C. After cooling to r.t. the mixture was concentrated under reduced pressure and the crude product was purified by preparative HPLC to give 57 mg (80% purity, 5.6% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-7 min 30-70% B, 6, rate 150 mL/min, temperature 25° C.

LC-MS (Method 2): Rt=1.09 min; MS (ESIpos): m/z=415 [M+H]⁺

¹H-NMR (400 MHz, DMSO-d₆): δ [ppm]=3.81 (d, 1H), 4.60 (d, 1H), 7.53-7.70 (m, 2H), 7.74 (d, 1H), 8.07 (d, 1H), 8.10 (d, 2H), 8.25 (d, 2H), 8.93 (d, 1H), 9.03 (d, 1H), 12.06 (br s, 1H).

### Intermediate 14-4

### 6-(4-Aminophenyl)-4-(quinolin-5-yl)-4-(trifluoromethyl)-4,5-dihydropyridazin-3(2H)-one

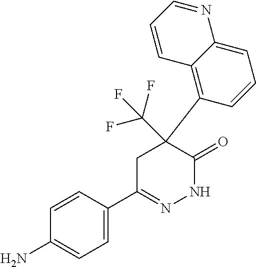 [SMILES: Nc1ccc(C2=NNC(=O)C(c3cccc4ncccc34)(C(F)(F)F)C2)cc1]

To 6-(4-nitrophenyl)-4-(quinolin-5-yl)-4-(trifluoromethyl)-4,5-dihydropyridazin-3(2H)-one (57.0 mg, 138 μmol) in methanol (2.8 mL) and ethyl acetate (5.0 mL) was added platinum/vanadium on activated carbon (26.8 mg, Pt 1%/V 2%). The mixture was stirred under a hydrogen atmosphere for 5 h at r. t. then additional platinum/vanadium on activated carbon (14 mg, Pt1/V2%) was added and stirring under hydrogen was continued for 7 h, The hydrogen atmosphere was exchanged to argon and the mixture was filtered through a short path of celite, which was thoroughly washed with methanol and THE afterwards. The combined filtrates were concentrated under reduced pressure to yield 66 mg (70% purity, 87% yield) of the title compound.

LC-MS (Method 2): Rt=0.95 min; MS (ESpos): m/z=385 [M+H]⁺

¹H-NMR (400 MHz, CDCl₃): δ [ppm]=3.51 (d, 1H), 3.98 (br s, 2H), 4.04 (d, 1H), 6.66-6.72 (m, 2H), 7.43 (dd, 1H), 7.52-7.63 (m, 3H), 8.13 (d, 1H), 8.61 (s, 1H), 8.91 (dd, 1H), 9.00 (d, 1H).

### Intermediate 14-5

### 4-Nitrophenyl {4-[6-oxo-5-(quinolin-5-yl)-5-(trifluoromethyl)-1,4,5,6-tetrahydropyridazin-3-yl]phenyl}carbamate

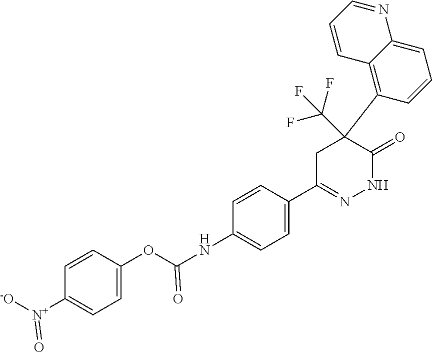

To 6-(4-nitrophenyl)-4-(quinolin-5-yl)-4-(trifluoromethyl)-4,5-dihydropyridazin-3(2H)-one (64.0 mg, 154 μmol) in THE (3.1 mL) was added at r.t. 4-nitrophenyl carbonochloridate (CAS-No. 7693-46-1, 62.3 mg, 309 μmol) in 4 equal portions and while the mixture was heated to 60° C. After 1.5 h the mixture was concentrated under reduced pressure to give the raw title compound (quant.), which was directly used in the next step.

### Example 14

### N-{4-[6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide

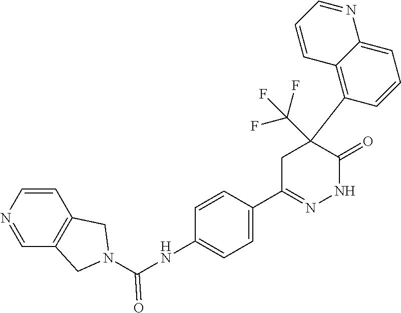 [SMILES: O=C(Nc1ccc(C2=NNC(=O)C(c3cccc4ncccc34)(C(F)(F)F)C2)cc1)N1Cc2ccncc2C1]

2,3-Dihydro-1H-pyrrolo[3,4-c]pyridine dihydrochloride (CAS-No. 6000-50-6, 45 mg, 232 μmol) was suspended in dichloromethane (1.0 mL) and N,N-diisopropylethylamine (130 μl, 770 μmol) and after stirring for 1 h added to a suspension of raw 4-nitrophenyl {4-[6-oxo-5-(quinolin-5-yl)-5-(trifluoromethyl)-1,4,5,6-tetrahydropyridazin-3-yl]phenyl}carbamate (intermediate 14-5, 85.0 mg, 155 μmol) in dichloromethane (2.2 mL). After stirring at r. t. for 30 min the mixture was concentrated under reduced pressure and purified by preparative HPLC to give 30 mg (85% purity, 31% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-6 min 15-55% B, 6-8 min 55-100% B, rate 150 mL/min, temperature 25° C.

LC-MS (Method 2): Rₜ=0.94 min; MS (ESIpos): m/z=531 [M+H]⁺

¹H-NMR (400 MHz, DMSO-d6) δ [ppm]: 3.60 (d, 1H), 4.47 (br d, 1H), 4.82 (br d, 4H), 7.44 (d, 1H), 7.59 (dd, 1H), 7.62-7.69 (m, 3H), 7.73-7.83 (m, 3H), 8.05 (d, 1H), 8.51 (d, 1H), 8.63 (d, 2H), 8.92 (dd, 1H), 9.11 (br d, 1H), 11.66 (s, 1H).

Chiral LC: Instrument: Agilent HPLC 1260; column: Chiralpak IG 3μ 100×4.6 mm; eluent A: Hexan+0.1 vol-% diethylamin; eluent B: ethanol; gradient: 20-50% B in 7 min; flow 1.4 mL/min; temperature: 25° C.; DAD @ 254 nm: Rt=2.86 min (37.5%)+3.12 min (38.1%), racemic mixture.

### Intermediate 15-1

### Tert-Butyl {6-[(3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]hexyl}carbamate

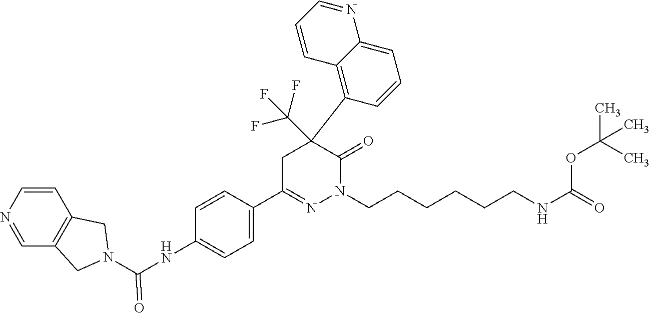 [SMILES: CC(C)(C)OC(=O)NCCCCCCN1N=C(c2ccc(NC(=O)N3Cc4ccncc4C3)cc2)CC(c2cccc3ncccc23)(C(F)(F)F)C1=O]

To a suspension of N-{4-[6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide (see Example 14, 27.2 mg, 51.3 μmol) in DMSO (490 μl)/DMF (490 μl) was added at r.t. sodium hydride (4.31 mg, 60% in mineral oil, 108 μmol) and stirred for 20 min. Then tetra-n-butylammonium iodide (1.89 mg, 5.13 μmol) and tert-butyl (6-bromohexyl)carbamate (26 μl, 110 μmol) were added at 0° C. After stirring for 1 h at r.t. water and DMSO were added to the mixture and it was purified by preparative HPLC to give 8.2 mg (90% purity, 19% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-7 min 30-70% B, rate 150 mL/min, temperature 25° C.

LC-MS (Method 2): Rt=1.28 min; MS (ESIpos): m/z=730 [M+H]⁺

1H-NMR (400 MHz, DMSO-d6) δ [ppm]: 0.97-1.09 (m, 2H), 1.10-1.13 (m, 2H), 1.13-1.25 (m, 2H), 1.36 (s, 9H) 1.41-1.49 (m, 2H), 2.81 (q, 2H), 3.54-3.67 (m, 1H), 3.63 (d, 1H), 3.85 (dt, 1H), 4.52 (d, 1H), 4.83 (br d, 4H), 6.74 (t, 1H), 7.39-7.49 (m, 1H), 7.57 (dd, 1H), 7.60-7.69 (m, 3H), 7.72-7.87 (m, 3H), 8.04 (d, 1H), 8.51 (d, 1H), 8.62 (s, 1H), 8.68 (s, 1H), 8.90 (dd, 1H), 9.05 (br d, 1H).

### Example 15

### N-{4-[1-(6-Aminohexyl)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide

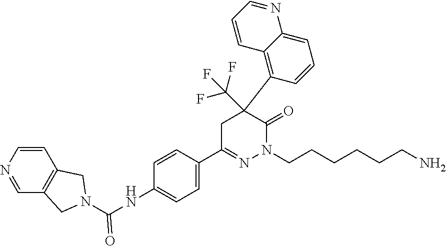 [SMILES: NCCCCCCN1N=C(c2ccc(NC(=O)N3Cc4ccncc4C3)cc2)CC(c2cccc3ncccc23)(C(F)(F)F)C1=O]

Tert-butyl {6-[3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]hexyl}carbamate (12.2 mg, 16.7 μmol) was stirred in dichloromethane (210 μL) together with trifluoroacetic acid (21 μL, 270 μmol) for 3 h. The mixture was concentrated under reduced pressure and purified by preparative HPLC to give 5.50 mg (95% purity, 50% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm Eluent A: water+0.1% formic acid; Eluent B: acetonitrile; gradient: 0-14 min 10-50% B, rate 60 mL/min, temperature 25° C.

LC-MS (Method 1): Rt=0.75 min; MS (ESIneg): m/z=628 [M−H]⁻

¹H-NMR (400 MHz, DMSO-d6): δ [ppm]: 0.97-1.05 (m, 2H), 1.08-1.20 (m, 2H), 1.26-1.37 (m, 2H), 1.41-1.53 (m, 2H), 2.60 (br t, 2H), 3.55-3.69 (m, 1H), 3.63 (d, 1H), 3.87 (dt, 1H), 4.52 (br d, 1H), 4.83 (br d, 4H), 7.44 (d, 1H), 7.58 (dd, 1H), 7.60-7.70 (m, 3H), 7.76 (br d, 1H), 7.83 (d, 2H), 8.05 (d, 1H), 8.37 (br s, 1H), 8.51 (d, 1H), 8.62 (s, 1H), 8.70 (s, 1H), 8.91 (dd, 1H), 9.05 (d, 1H).

### Intermediate 16-1

### 5-Bromo-1-(oxan-2-yl)-1H-indazole

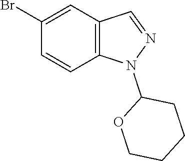 [SMILES: Brc1ccc2c(cnn2C2CCCCO2)c1]

To a solution of 5-bromo-1H-indazole (CAS 53857-57-1, 2.0 g, 10.15 mmol) in dichloromethane (40 mL), 3,4-dihydro-2H-pyran (CAS 110-87-2, 1.85 mL, 20.3 mmol) and 4-toluenesulfonic acid monohydrate (0.96 g, 5.07 mmol) were added and the reaction was stirred at r.t. for 3 h. To this mixture saturated sodium bicarbonate solution was added and the dichloromethane layer was collected, washed with water, brine and dried over sodium sulfate. The organic layer was concentrated under vacuum and the crude product was purified by flash chromatography on silica 60 (eluent: ethyl acetate-heptane 1:7, 1:6) to give 1.6 g (98% purity, 55% yield) and 1 g (85% purity, 30% yield) as two batches of the title compound.

LC-MS (Method 5): Rt=0.89 min; MS (ESIpos): m/z=197 [M+H]⁺, 199 [M+H]⁺, (pyran group fragmented in the mass spec)

¹H NMR (400 MHz, CDCl₃) δ [ppm]: 1.50-1.77 (m, 3H), 2.08-2.14 (m, 2H), 2.50-2.55 (m, 1H), 3.73-3.77 (m, 1H), 3.98-4.00 (m, 1H), 5.63-5.71 (m, 1H), 7.44-7.52 (m, 2H), 7.82 (s, 1H), 8.08 (s, 1H)

### Intermediate 16-2

### 1-(Oxan-2-yl)-5-(4,4,5,5-tetramethyl-1,3,2-dioxaborolan-2-yl)-1H-indazole

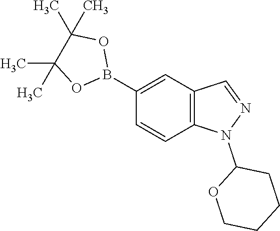 [SMILES: CC1(C)OB(c2ccc3c(cnn3C3CCCCO3)c2)OC1(C)C]

A mixture of 5-bromo-1-(oxan-2-yl)-1H-indazole (1.6 g, 5.69 mmol), 4,4,5,5-tetramethyl-1,3,2-dioxaborolan-2-yl-4′,4′,5′,5′-tetramethyl-1,3,2-dioxaborolane (1.59 g, 6.26 mmol), potassium acetate (1.67 g, 17.0 mmol) and bis(diphenylphosphino)ferrocene]dichloropalladium (208 mg, 0.28 mmol) in dry dioxane (30 mL) was degassed under argon. The mixture was heated at 110° C. for 5 h. This mixture was filtered through celite and washed with ethyl acetate. The filtrate was concentrated under vacuum to give the crude product. This was purified by flash chromatography (SiO₂, ethyl acetate/heptane, gradient) to give 2.4 g (84% purity, 79% yield). the desired product as a colourless oil.

LC-MS (Method 5): Rt=0.96 min, MS (ESIpos): m/z=245 [M+H]⁺, (pyran group fragmented in the mass spec)

¹H NMR (400 MHz, CDCl₃) [ppm]: 1.35 (s, 12H), 1.57-1.65 (m, 1H), 1.73-1.78 (m, 2H), 2.04-2.07 (m, 1H), 2.14-2.15 (m, 1H), 2.53-2.59 (m, 1H), 3.71-3.77 (m, 1H), 4.01-4.04 (m, 1H), 5.70-5.73 (m, 1H), 7.53-7.56 (m, 1H), 7.77-7.80 (m, 1H), 8.02 (s, 1H), 8.22 (s, 1H).

### Intermediate 16-3

### 4-Bromo-6-chloropyridazin-3(2H)-one

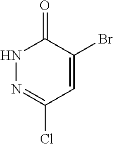

6-Chloropyridazin-3(2H)-one (CAS 19064-67-6, 10.0 g, 76.6 mmol) was suspended in water (100 mL), potassium bromide (27.3 g, 230 mmol) and sodium acetate (9.43 g, 115 mmol) were added at r.t. Bromine (12 mL, 230 mmol) was added and the orange suspension was heated under reflux for 2 h. After 2 h the mixture was left to cool down to r.t. and the solid was filtered, washed with sodium sulfite (10% aqueous solution) and water. Then it was dried in vacuum to give 12.78 g of the title compound (95% purity, 75% yield) as beige solid. LC-MS (Method 5): Rt=0.63 min; MS (ESIneg): m/z=206 [M−H]⁻, 208 [M−H]⁻, 210 [M−H]⁻

¹H-NMR (400 MHz, CDCl₃) δ [ppm]: 7.67 (s, 1H), 10.75 (br s, 1H).

### Intermediate 16-4

### 6-Chloro-4-{1-[oxan-2-yl]-1H-indazol-5-yl}pyridazin-3(2H)-one

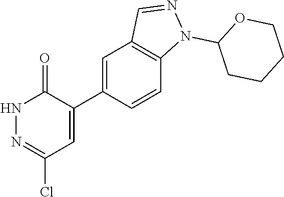 [SMILES: O=c1[nH]nc(Cl)cc1-c1ccc2c(cnn2C2CCCCO2)c1]

A mixture of 4-bromo-6-chloropyridazin-3(2H)-one (see Intermediate 16-3, 397 mg, 1.89 mmol), 1-(tetrahydro-2H-pyran-2-yl)-5-(4,4,5,5-tetramethyl-1,3,2-dioxaborolan-2-yl)-1H-indazole (see Intermediate 16-2, 622 mg, 1.89 mmol), potassium carbonate (393 mg, 2.84 mmol) were suspended in dioxane/water (5:1, 48 mL) and the mixture was degassed for 10 min. Bis(diphenylphosphino)ferrocene]dichloropalladium (69 mg, 0.095 mmol) was added and the mixture was heated to 90° C. for 2 h. LC-MS showed 86% product area. This mixture is used directly for the next step without purification.

### Intermediate 16-5

### 6-(4-Aminophenyl)-4-{1-[oxan-2-yl]-1H-indazol-5-yl}pyridazin-3(2H)-one

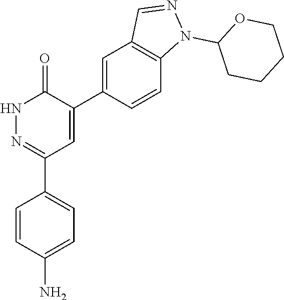 [SMILES: Nc1ccc(-c2cc(-c3ccc4c(cnn4C4CCCCO4)c3)c(=O)[nH]n2)cc1]

To the reaction mixture from intermediate 16-4 was added 4-aminophenylboronic acid pinacol ester (497 mg, 2.27 mmol) and bis(diphenylphosphino)ferrocene]dichloropalladium (69 mg, 0.095 mmol) and the mixture was heated at 110° C. for 16 h. Then it was cooled to rt and partitioned between ethyl acetate and saturated sodium bicarbonate solution. The ethyl acetate layer was washed with brine, dried over sodium sulfate and concentrated to give the crude product, which was purified by flash chromatography (eluent: heptane/ethyl acetate 1:1, ethyl acetate-methanol 95:5) to give 460 mg of the desired product (92% purity, 58% yield) as pale yellow solid.

LC-MS (Method 3): Rt=0.62 min; MS (ESIpos): m/z=388 [M+H]⁺

¹H NMR (400 MHz, CDCl₃) δ [ppm]: 1.61-1.77 (m, 3H), 2.07-2.16 (m, 2H), 2.56-2.58 (m, 1H), 3.73-3.77 (m, 1H), 3.89 (br s, 2H), 4.01-4.04 (m, 1H), 5.74-5.77 (m, 1H), 6.74-6.76 (d, 2H), 7.63-7.69 (m, 3H), 7.80 (s, 1H), 7.85-7.88 (m, 1H), 8.10 (s, 1H), 8.36 (s, 1H), 10.50 (br, s, 1H);

### Intermediate 16-6

### 4-Nitrophenyl [4-(5-{1-[oxan-2-yl]-1H-indazol-5-yl}-6-oxo-1,6-dihydropyridazin-3-yl)phenyl]carbamate

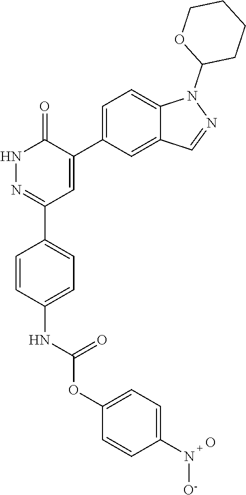 [SMILES: O=C(Nc1ccc(-c2cc(-c3ccc4c(cnn4C4CCCCO4)c3)c(=O)[nH]n2)cc1)Oc1ccc(N[O-])cc1 ; O]

6-(4-Aminophenyl)-4-{1-[oxan-2-yl]-1H-indazol-5-yl}pyridazin-3(2H)-one (460 mg, 1.19 mmol) was dissolved in a mixture of dichloromethane (44 mL) and dimethylformamide (20 mL) at r.t. under argon and pyridine (190 μl, 2.4 mmol) was added. 4-nitrophenyl carbonochloridate (287 mg, 1.42 mmol) was added in portions within 1 min. The mixture was stirred at r.t. until all the starting material was consumed. This mixture was used directly in the experiment of Intermediate 16-7.

### Intermediate 16-7

### N-[4-(5-{1-[Oxan-2-yl]-1H-indazol-5-yl}-6-oxo-1,6-dihydropyridazin-3-yl)phenyl]-1,3-dihydro-2H-pyrrolo[3,4-c]pyridine-2-carboxamide

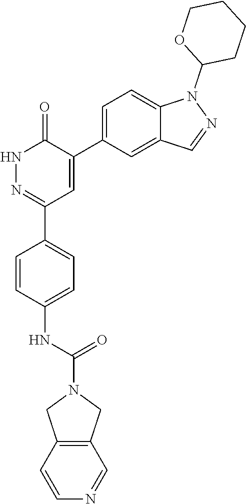 [SMILES: O=C(Nc1ccc(-c2cc(-c3ccc4c(cnn4C4CCCCO4)c3)c(=O)[nH]n2)cc1)N1Cc2ccncc2C1]

N,N-Diisopropylethylamine (1.02 mL, 5.9 mmol) followed by 2,3-dihydro-1H-pyrrolo[3,4-c]pyridine hydrochloride (455 mg, 2.36 mmol) was added to the reaction mixture from experiment of Intermediate 16-6 at r.t. under argon. The mixture was stirred at r.t. for 5 h. This mixture was concentrated under vacuum. The residue was triturated with water/ethanol mixture and the solid formed was collected by filtration, washed with ethyl acetate, ether and dried to give 510 mg of the desired product (99% purity, 80% yield) as pale yellow solid.

LC-MS (Method 3): Rt=0.47 min; MS (ESIpos): m/z=534 [M+H]⁺

¹H-NMR (400 MHz, DMSO-D₆) δ [ppm]: 1.57 (s, 2H), 1.74 (s, 1H), 1.95-2.03 (m, 2H), 2.38-2.46 (m, 1H), 3.70-3.77 (m, 1H), 3.85-3.87 (m, 1H), 4.79-4.81 (m, 4H), 5.86-5.99 (m, 1H), 7.40-7.41 (m, 1H), 7.68-7.70 (m, 2H), 7.78 (d, 1H), 7.87-7.89 (m, 2H), 8.02 (d, 1H), 8.17 (d, 2H), 8.46-8.47 (m, 1H), 8.52 (s, 1H), 8.59 (s, 2H).

### Example 16

### N-{4-[5-(1H-indazol-5-yl)-6-oxo-1,6-dihydropyridazin-3-yl]phenyl}-1,3-dihydro-2H-pyrrolo[3,4-c]pyridine-2-carboxamide-hydrogen chloride

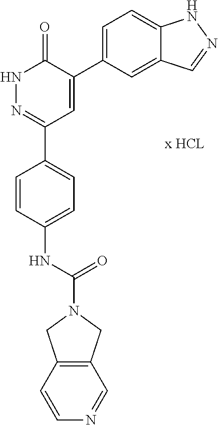 [SMILES: O=C(Nc1ccc(-c2cc(-c3ccc4[nH]ncc4c3)c(=O)[nH]n2)cc1)N1Cc2ccncc2C1]

To a mixture of N-(4-{5-[1-(oxan-2-yl)-1H-indazol-5-yl]-6-oxo-1,6-dihydropyridazin-3-yl}phenyl)-1,3-dihydro-2H-pyrrolo[3,4-c]pyridine-2-carboxamide (50.0 mg, 93.7 μmol) in 4M HCl/dioxane (5.0 mL) was added water and stirred at r.t. for 4 h. The mixture was concentrated under vacuum and the residue was triturated with methanol which after drying gave 30 mg of the desired product (95% purity, 63% yield) as hydrochloride salt.

LC-MS (Method 4): Rt=1.06 min; MS (ESIpos): m/z=450 [M+H]⁺

1H-NMR (400 MHz, DMSO-D6): δ [ppm] 4.94-5.0 (m, 4H), 7.57-7.69 (m, 3H), 7.88-8.01 (m, 4H), 8.13-8.16 (m, 2H), 8.52 (s, 1H), 8.79 (s, 2H), 8.93 (s, 1H)

### Example 17

### N-{4-[5-(1H-indazol-5-yl)-6-oxo-1,4,5,6-tetrahydropyridazin-3-yl]phenyl}-1,3-dihydro-2H-pyrrolo[3,4-c]pyridine-2-carboxamide

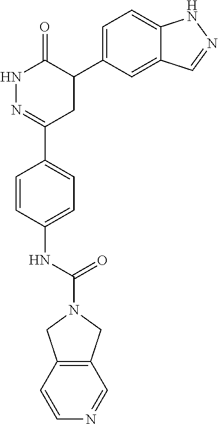 [SMILES: O=C1NN=C(c2ccc(NC(=O)N3Cc4ccncc4C3)cc2)CC1c1ccc2[nH]ncc2c1]

N-{4-[5-(1H-Indazol-5-yl)-6-oxo-1,6-dihydropyridazin-3-yl]phenyl}-1,3-dihydro-2H-pyrrolo[3,4-c]pyridine-2-carboxamide (see Example 16, 80.0 mg, 178 μmol) was dissolved in acetic acid (5 mL). The mixture was heated with vigorous stirring at 90° C. in a preheated bath for 2 min. Zinc dust (58 mg, 0.89 mmol) was added and the mixture was heated at 90° C. The mixture was concentrated under vacuum, the resulting residue was dissolved in DMSO and filtered. The filtrate was purified by MDAP ((XBridge C18, 19×150 mm, 5 μm, 0.1% formic acid in water-acetonitrile; 35% isocraticover 11 mins) followed by freeze drying afforded 31 mg of the desired product (97% purity, 37% yield) as a pale yellow gum.

LC-MS (Method 4): Rt=0.73 min; MS (ESIpos): m/z=452 [M+H]⁺

¹H-NMR (400 MHz, DMSO-D6): δ [ppm]: 3.17-3.28 (m, 2H), 3.88-3.92 (m, 1H), 4.84-4.85 (m, 4H), 7.18-7.26 (m, 1H), 7.45-7.47 (m, 1H), 7.57-7.60 (m, 3H), 7.65-7.69 (m, 3H), 7.99 (d, 1H), 8.60-8.63 (m, 2H), 8.72 (s, 1H), 11.06 (s, 1H).

### Intermediate 18-1

### 4-Bromo-1-(oxan-2-yl)-1H-indazole

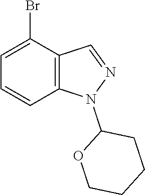 [SMILES: Brc1cccc2c1cnn2C1CCCCO1]

4-Bromo-IH-indazole (CAS 186407-74-9, 2.0 g, 5.07 mmol) was dissolved in anhydrous tetrahydrofuran (50 mL). To this solution was added 3,4-dihydro-2H-pyran (CAS 110-87-2, 1.85 mL, 20.3 mmol) and toluenesulfonic acid monohydrate (0.96 g, 5.07 mmol). The reaction mixture was refluxed for 16 h. Saturated bicarbonate was added and the aqueous layer was extracted with ethyl acetate (3×50 mL). The organic layer was washed with brine, dried over sodium sulfate and concentrated to give the crude product. This was purified by flash chromatography (eluent: ethyl acetate-heptane 1:9) to give 2.65 g of the desired product (77% purity, 72% yield) as colourless oil, which solidified upon standing.

LC-MS (Method 5): Rt=0.92 min, MS (ESIpos): m/z=197 [M+H]⁺, 199 [M+H]⁺, (pyran group fragmented in the mass spec)

1H NMR (400 MHz, CDCl3) [ppm]: 1.50-1.77 (m, 3H), 2.04-2.16 (m, 2H), 2.52-2.55 (m, 1H), 3.73-3.77 (m, 1H), 3.98-4.00 (m, 1H), 5.63-5.71 (m, 1H), 7.11-7.27 (m, 1H), 7.30-7.34 (d, 1H), 7.48-7.55 (d, 1H), 8.08 (s, 1H).

### Intermediate 18-2

### 1-(Oxan-2-yl)-4-(4,4,5,5-tetramethyl-1,3,2-dioxaborolan-2-yl)-1H-indazole

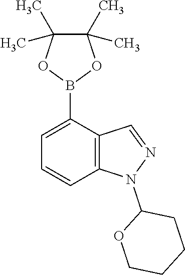 [SMILES: CC1(C)OB(c2cccc3c2cnn3C2CCCCO2)OC1(C)C]

A mixture of 5-bromo-1-(tetrahydro-2H-pyran-2-yl)-1H-indazole, (2.60 g, 9.25 mmol), 4,4,4′,4′,5,5,5′,5′-octamethyl-2,2′-bi-1,3,2-dioxaborolane, (2.59 g, 10.2 mmol), potassium acetate, (2.71 g, 17.1 mmol), and bis(diphenylphosphino)ferrocene]dichloropalladium(II), (338 mg, 0.46 mmol), in 1,4-dioxane, 48.0 mL, was degassed with argon for 10 minutes. The reaction mixture was heated at 110° C. for 5 hours, cooled to r.t. and filtered through celite, washed with ethyl acetate. The filtrate was concentrated and combined with another batch starting from 5-bromo-1-(tetrahydro-2H-pyran-2-yl)-1H-indazole, 1.00 g (3.56 mmol). Purification by column chromatography (45 g; ZIP Sphere Silica Biotage cartridge, ethyl acetate/heptane gradient) gave the desired product, 2.40 g ((97% purity, 79% yield).

LC-MS (Method 5): Rt=0.97 min; MS (ESIpos): m/z=245 [M+H]⁺

¹H NMR (400 MHz, CDCl₃) [ppm]: 1.35 (s, 12H), 1.57-1.65 (m, 1H), 1.73-1.78 (m, 2H), 2.04-2.07 (m, 1H), 2.14-2.15 (m, 1H), 2.53-2.59 (m, 1H), 3.71-3.77 (m, 1H), 4.01-4.04 (m, 1H), 5.70-5.73 (m, 1H), 7.39-7.40 (m, 1H), 7.57-7.59 (d, 1H), 7.70-7.73 (d, 1H), 8.39 (s, 1H).

### Intermediate 18-3

### 6-chloro-4-{1-[oxan-2-yl]-1H-indazol-4-yl}pyridazin-3(2H)-one

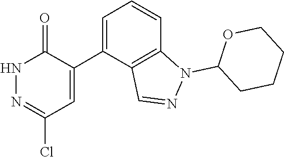 [SMILES: O=c1[nH]nc(Cl)cc1-c1cccc2c1cnn2C1CCCCO1]

A mixture of 4-bromo-6-chloropyridazin-3(2H)-one (see Intermediate 16-3, 600 mg, 2.86 mmol), 1-(tetrahydro-2H-pyran-2-yl)-4-(4,4,5,5-tetramethyl-1,3,2-dioxaborolan-2-yl)-1H-indazole (see Intermediate 18-1

### 4-Bromo-1-(oxan-2-yl)-1H-indazole

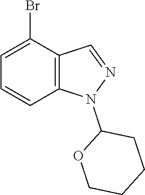 [SMILES: Brc1cccc2c1cnn2C1CCCCO1]

4-Bromo-IH-indazole (CAS 186407-74-9, 2.0 g, 5.07 mmol) was dissolved in anhydrous tetrahydrofuran (50 mL). To this solution was added 3,4-dihydro-2H-pyran (CAS 110-87-2, 1.85 mL, 20.3 mmol) and toluenesulfonic acid monohydrate (0.96 g, 5.07 mmol). The reaction mixture was refluxed for 16 h. Saturated bicarbonate was added and the aqueous layer was extracted with ethyl acetate (3×50 mL). The organic layer was washed with brine, dried over sodium sulfate and concentrated to give the crude product. This was purified by flash chromatography (eluent: ethyl acetate-heptane 1:9) to give 2.65 g of the desired product (77% purity, 72% yield) as colourless oil, which solidified upon standing.

LC-MS (Method 5): Rt=0.92 min; MS (ESipos): m/z=197 [M+H]⁺, 199 [M+H]⁺, (pyran group fragmented in the mass spec)

1H NMR (400 MHz, CDCl3) [ppm]: 1.50-1.77 (m, 3H), 2.04-2.16 (m, 2H), 2.52-2.55 (m, 1H), 3.73-3.77 (m, 1H), 3.98-4.00 (m, 1H), 5.63-5.71 (m, 1H), 7.11-7.27 (m, 1H), 7.30-7.34 (d, 1H), 7.48-7.55 (d, 1H), 8.08 (s, 1H).

Intermediate 18-2, 940 mg, 2.86 mmol), potassium carbonate (594 mg, 4.29 mmol) were suspended in dioxane/water (5:1, 72 mL) and the mixture was degassed for 10 min. Bis(diphenylphosphino)ferrocene]dichloropalladium (104 mg, 0.14 mmol) was added and the mixture was heated to 90° C. for 2 h. This mixture is used directly for experiment of Intermediate 18-4 for second suzuki coupling.

### Intermediate 18-4

### 6-(4-Aminophenyl)-4-{1-[oxan-2-yl]-1H-indazol-4-yl}pyridazin-3(2H)-one

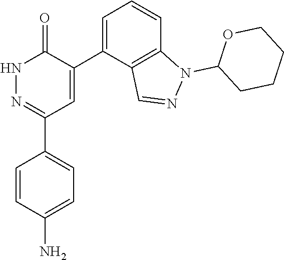 [SMILES: Nc1ccc(-c2cc(-c3cccc4c3cnn4C3CCCCO3)c(=O)[nH]n2)cc1]

To the reaction mixture from experiment of Intermediate 18-3 were added 4-aminophenylboronic acid pinacol ester (753 mg, 3.44 mmol) and bis(diphenylphosphino)ferrocene]dichloropalladium (104 mg, 0.14 mmol) and the mixture was heated at 110° C. for 5 h. After cool to r.t. it was partitioned between ethyl acetate and saturated sodium bicarbonate solution and the ethyl acetate layer was washed with brine, dried over sodium sulfate and concentrated to give the crude product. This was purified by flash chromatography (eluent: heptane-ethyl acetate 1:1, ethylacetate-methanol 95:5) to give the desired product in 2 batches of 360 mg (87% purity, 28% yield) and 330 mg (76% purity, 22% yield).

LC-MS (Method 3): Rt=0.62 min: MS (ESIpos): m/z=388 [M+H]⁺

1H-NMR (400 MHz, CDCl₃) δ [ppm]: 1.68-1.78 (m, 3H), 1.95-2.32 (m, 2H), 2.58-2.63 (m, 1H), 3.76-3.78 (m, 1H), 3.89 (br s, 2H), 4.01-4.04 (m, 1H), 5.76-5.79 (m, 1H), 6.73 (d, 2H), 7.45-7.53 (m, 1H), 7.61-7.65 (m, 3H), 7.66-7.73 (m, 1H), 7.92 (s, 1H), 8.12 (s, 1H), 10.95 (s, 1H).

### Intermediate 18-5

### N-[4-(5-{1-[Oxan-2-yl]-1H-indazol-4-yl}-6-oxo-1,6-dihydropyridazin-3-yl)phenyl]-1,3-dihydro-2H-pyrrolo[3,4-c]pyridine-2-carboxamide

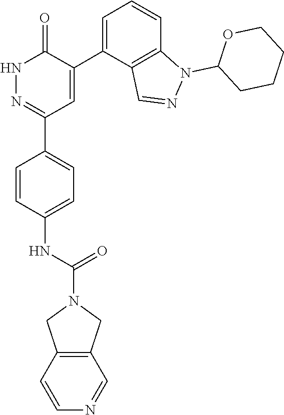 [SMILES: O=C(Nc1ccc(-c2cc(-c3cccc4c3cnn4C3CCCCO3)c(=O)[nH]n2)cc1)N1Cc2ccncc2C1]

To a solution of 6-(4-aminophenyl)-4-{1-[oxan-2-yl]-1H-indazol-4-yl}pyridazin-3(2H)-one (360 mg, 0.929 mmol) in dichloromethane (34.3 mL) under argon was added pyridine (150 μL, 1.86 mmol), followed by portion wise addition of 4-nitrophenyl carbonochloridate (225 mg 1.12 mmol) over 1 minute, and the reaction mixture stirred at r.t. for 5 hours. To this crude reaction mixture was added N,N-diisopropylethylamine (809 μL, 4.65 mmol), followed by 2,3-dihydro-1H-pyrrolo[3,4-c]pyridine dihydrochloride (359 mg, 1.86 mmol) under argon and the reaction mixture stirred at r.t. for 5 hours. The reaction mixture was combined with another batch starting from 4-nitrophenyl (4-{6-oxo-5-[1-(tetrahydro-2H-pyran-2-yl)-1H-indazol-4-yl]-1,6-dihydropyridazin-3-yl}phenyl)carbamate (0.852 mmol), and concentrated to give a residue. The crude residue was triturated with water-ethanol and the precipitate collected by filtration, washed with ethyl acetate, diethyl ether and dried to give the desired product, 580 mg (61% combined yield over two steps, 88% purity).

LC-MS (Method 3): Rt=0.46 min, MS (ESIneg): m/z=532 [M−H]⁻

1H-NMR (400 MHz, DMSO-d6) δ [ppm]: 1.57 (s, 2H), 1.75 (s, 1H), 1.94-2.01 (m, 2H), 2.40-2.42 (m, 1H), 3.70-3.77 (m, 1H), 3.85-3.88 (m, 1H), 4.80 (d, 4H), 5.87-5.89 (m, 1H), 7.41-7.42 (m, 1H), 7.47-7.52 (m, 2H), 7.66-7.74 (m, 2H), 7.80-7.83 (m, 3H), 8.08-8.10 (m, 2H), 8.48 (d, 1H), 8.59-8.62 (m, 2H).

### Example 18

### N-{4-[5-(1H-indazol-4-yl)-6-oxo-1,6-dihydropyridazin-3-yl]phenyl}-1,3-dihydro-2H-pyrrolo[3,4-c]pyridine-2-carboxamide

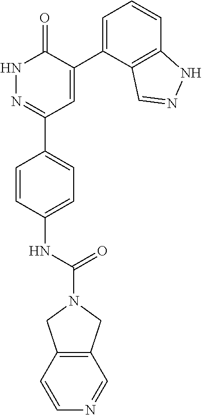 [SMILES: O=C(Nc1ccc(-c2cc(-c3cccc4[nH]ncc34)c(=O)[nH]n2)cc1)N1Cc2ccncc2C1]

To a mixture of N-[4-(5-{1-[oxan-2-yl]-1H-indazol-4-yl}-6-oxo-1,6-dihydropyridazin-3-yl)phenyl]-1,3-dihydro-2H-pyrrolo[3,4-c]pyridine-2-carboxamide (50.0 mg, 93.7 μmol) in 4M HCl/dioxane (5.0 mL) was added water (2.0 mL) and the mixture was stirred at r.t. for 4 h. The mixture was concentrated under vacuum, the residue was dissolved in DMSO and it was purified by preparative HPLC, followed by freeze drying afforded 9 mg of the desired product (98% purity, 21% yield) as colourless solid.

HPLC: Instrument: Waters mass directed auto-purification system, pump head/gradient module: 2545 Binary gradient module, fraction collector: 2767 Waters sample manager, 2998 Photodiode array detector, MassLynx software. Column: XBridge C18, 19×150 mm, 5 μm; Eluent A: 0.1% formic acid in water; Eluent B: acetonitrile; gradient: 15-20% over 10 mins; rate 20 mL/min, temperature 25° C.

LC-MS (Method 4): Rt=1.06 min; MS (ESIpos): m/z=450 [M+H]⁺

1H-NMR (400 MHz, DMSO-D6): δ (ppm)=4.78-4.80 (m, 4H), 7.40-7.49 (m, 3H), 7.60-7.68 (m, 3H), 7.83-7.85 (m, 2H), 8.06 (s, 1H), 8.11 (s, 1H), 8.46-8.47 (m, 1H), 8.58-8.61 (m, 2H).

### Intermediate 19-1

### N-{4-[5-{1-[Oxan-2-yl]-1H-indazol-4-yl}-6-oxo-1,4,5,6-tetrahydropyridazin-3-yl]phenyl}-1,3-dihydro-2H-pyrrolo[3,4-c]pyridine-2-carboxamide

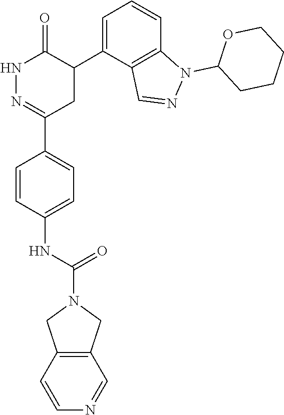 [SMILES: O=C1NN=C(c2ccc(NC(=O)N3Cc4ccncc4C3)cc2)CC1c1cccc2c1cnn2C1CCCCO1]

N-[4-(5-{1-[Oxan-2-yl]-1H-indazol-4-yl}-6-oxo-1,6-dihydropyridazin-3-yl)phenyl]-1,3-dihydro-2H-pyrrolo[3,4-c]pyridine-2-carboxamide (see Intermediate 18-, 100 mg, 187 μmol) was dissolved in acetic acid (10 mL) and the mixture was heated with vigorous stirring at 90° C. in a preheated bath for 2 min. Zinc dust (61.3 mg, 937 μmol) was added and the mixture was heated at 90° C. The mixture was filtered through celite, washed with acetic acid and the filtrate was concentrated under vacuum. The residue was purified by preparative HPLC, followed by freeze drying afforded 30 mg of the desired product (95% purity, 28% yield) as colourless solid.

HPLC: Instrument: Waters mass directed auto-purification system, pump head/gradient module: 2545 Binary gradient module, fraction collector: 2767 Waters sample manager, 2998 Photodiode array detector, MassLynx software. Column: XBridge C18, 19×150 mm, 5 μm; Eluent A: 0.1% ammonium hydroxide in water; Eluent B: acetonitrile; 30% isocratic; rate 20 mL/min, temperature 25° C.

LC-MS (Method 4): Rt=1.11 min; MS (ESIneg): m/z=534 [M−H]⁻

1H-NMR (400 MHz, DMSO-D6): δ (ppm)=1.55 (s, 2H), 1.72 (s, 1H), 1.93-1.98 (m, 2H), 2.37-2.46 (m, 3H), 3.67-3.74 (m, 1H), 3.82-3.87 (m, 1H), 4.26-4.31 (m, 1H), 4.77 (d, 4H), 5.72-5.82 (m, 1H), 6.96-6.98 (m, 1H), 7.25-7.33 (m, 1H), 7.40 (d, 1H), 7.51-7.61 (m, 3H), 7.65-7.68 (m, 2H), 8.21 (s, 1H), 8.46 (d, 1H), 8.57 (s, 2H), 11.12 (d, 1H).

### Example 19

### N-{4-[5-(1H-indazol-4-yl)-6-oxo-1,4,5,6-tetrahydropyridazin-3-yl]phenyl}-1,3-dihydro-2H-pyrrolo[3,4-c]pyridine-2-carboxamide

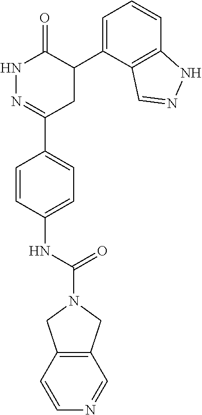 [SMILES: O=C1NN=C(c2ccc(NC(=O)N3Cc4ccncc4C3)cc2)CC1c1cccc2[nH]ncc12]

To a mixture of N-{4-[5-{1-[oxan-2-yl]-1H-indazol-4-yl}-6-oxo-1,4,5,6-tetrahydropyridazin-3-yl]phenyl}-1,3-dihydro-2H-pyrrolo[3,4-c]pyridine-2-carboxamide (23.0 mg, 42.9 μmol), in hydrochloric acid (4 M in 1,4-dioxane), 5.00 mL, was added water, 2.00 mL, and the reaction mixture stirred at room temperature for 4 hours then concentrated to give a residue. The crude residue was purified by MDAP (XSelect CSH C18, 19×150 mm, 5 μm, 0.1% formic acid in water-acetonitrile; 15-20% over 10 min.) to give 8.00 mg (99% purity, 49% yield) of the desired product.

HPLC: Instrument: Waters mass directed auto-purification system, pump head/gradient module: 2545 Binary gradient module, fraction collector: 2767 Waters sample manager, 2998 Photodiode array detector, MassLynx software. Column: XSelect CSH C18, 19×150 mm, 5 μm; Eluent A: 0.1% formic acid in water; Eluent B: acetonitrile; gradient: 15-20% over 10 mins; rate 20 mL/min, temperature 25° C.

LC-MS (Method 6): Rt=1.17 min; MS (ESIpos): m/z=452 [M+H]⁺

1H-NMR (400 MHz, DMSO-D6): δ (ppm)=3.29-3.31 (m, 2H), 4.24-4.26 (m, 1H), 4.85-4.87 (m, 4H), 6.87-6.89 (m, 1H), 7.23-7.25 (m, 1H), 7.41-7.43 (m, 2H), 7.57-7.67 (m, 4H), 8.16 (s, 1H), 8.47 (s, 1H), 8.57 (s, 2H), 11.11 (s, 1H).

### Intermediate 20-1

### 1-(4-methylbenzene-1-sulfonyl)-5-(4,4,5,5-tetramethyl-1,3,2-dioxaborolan-2-yl)-1H-indole

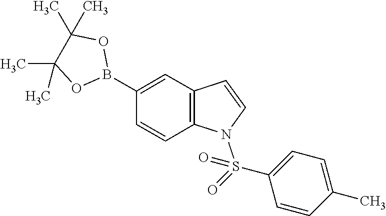

5-(4,4,5,5-tetramethyl-1,3,2-dioxaborolan-2-yl)-1H-indole (CAS 269410-24-4, 1.0 g, 4.1 mmol) was added to a round bottom flask, to which was added tetrahydrofuran (50 mL) under argon. Sodium hydride (60% dispersion in mineral oil, 246 mg, 6.17 mmol) was added and stirred for 30 min. The reaction mixture was then cooled to 0° C. and tosyl chloride (941 mg, 4.93 mmol) was added. The mixture was allowed to warm to r.t. and stirred for 16 h. Then the crude mixture was quenched with saturated ammonium chloride solution and concentrated under vacuum. The residue was partitioned between water and dichloromethane. The dichloromethe layer was collected, dried over sodium sulfate and concentrated in vacuum. The crude product was purified by Isolera (45 g, eluent: heptane/ethyl acetate, 9:1, 3:1) to give 1.0 g of the desired product (99% purity, 61% yield) as colourless solid.

LC-MS (Method 3): Rt=0.85 min; MS (ESIpos): m/z=398 [M+H]⁺

1H-NMR (400 MHz, CDCl₃) δ [ppm]: 1.32 (s, 12H), 2.34 (s, 3H), 6.64 (dd, 1H), 7.15 (d, 2H), 7.54 (d, 1H), 7.73 (d, 3H), 7.96 (d, 1H), 8.01 (s, 1H).

### Intermediate 20-2

### 6-(4-Aminophenyl)-4-[1-(4-methylbenzene-1-sulfonyl)-1H-indol-5-yl]pyridazin-3(2H)-one

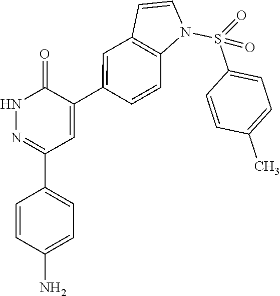 [SMILES: Cc1ccc(S(=O)(=O)n2ccc3cc(-c4cc(-c5ccc(N)cc5)n[nH]c4=O)ccc32)cc1]

A mixture of 4-bromo-6-chloropyridazin-3(2H)-one (Intermediate 16-3, 451 mg, 2.16 mmol), 1-[(4-methylphenyl)sulfonyl]-5-(4,4,5,5-tetramethyl-1,3,2-dioxaborolan-2-yl)-1H-indole (856 mg, 2.16 mmol), and potassium carbonate (447 mg, 3.23 mmol) in 1,4-dioxane (50.0 mL) and water (10.0 mL) was degassed with argon for 10 minutes. Bis(diphenylphosphino)ferrocene] dichloropalladium(II) (78.8 mg, 0.108 mmol) was added and the reaction mixture heated at 90° C. for 2 hours. To this crude reaction mixture was added 4-(4,4,5,5-tetramethyl-1,3,2-dioxaborolan-2-yl)aniline (567 mg, 2.59 mmol) and bis(diphenylphosphino)ferrocene] dichloropalladium(II) (78.0 mg, 0.11 mmol) and the reaction mixture heated at 110° C. for 22 hours. The reaction mixture was cooled to r.t. and combined with another batch (0.50 mmol). The combined reaction mixtures were partitioned between ethyl acetate and saturated sodium hydrogen carbonate solution, the organic layer separated and concentrated to give a residue. The crude residue was purified by column chromatography (45 g; ZIP Sphere Silica Biotage cartridge, ethyl acetate/heptane gradient) to give 300 mg of the desired product (96% purity, 21% combined yield) as pale brown solid.

LC-MS (Method 3): Rt=0.83 min; MS (ESIpos): m/z=457 [M+H]⁺

1H-NMR (400 MHz, CHLOROFORM-D) δ (ppm): 2.34 (s, 3H), 3.89 (br s, 2H), 6.71-6.75 (m, 3H), 7.22-7.24 (m, 2H), 7.59-7.63 (m, 3H), 7.72-7.77 (m, 4H), 8.05-8.12 (m, 2H), 10.68 (s, 1H).

### Intermediate 20-3

### N-(4-{5-[1-(4-Methylbenzene-1-sulfonyl)-1H-indol-5-yl]-6-oxo-1,6-dihydropyridazin-3-yl}phenyl)-1,3-dihydro-2H-pyrrolo[3,4-c]pyridine-2-carboxamide

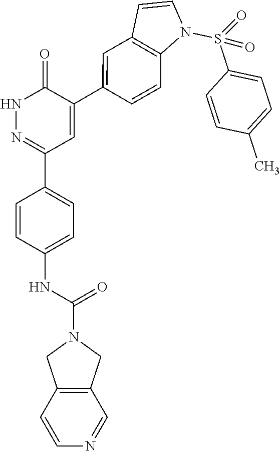 [SMILES: Cc1ccc(S(=O)(=O)n2ccc3cc(-c4cc(-c5ccc(NC(=O)N6Cc7ccncc7C6)cc5)n[nH]c4=O)ccc32)cc1]

To a solution of 6-(4-aminophenyl)-4-{1-[(4-methylphenyl)sulfonyl]-1H-indol-5-yl}pyridazin-3(2H)-one (200 mg, 0.438 mmol) in dichloromethane (16.2 mL) and N,N-dimethylformamide (4.00 mL) was added pyridine (71.0 μL, 0.876 mmol) followed by portion wise addition of 4-nitrophenyl carbonochloridate (106 mg, 0.526 mmol) over 1 minute, and the reaction mixture stirred at r.t. for 1 hour. To this reaction mixture was added N,N-diisopropylethylamine (381 μL, 2.19 mmol) followed by 2,3-dihydro-1H-pyrrolo[3,4-c]pyridine dihydrochloride (169 mg, 0.876 mmol) under argon and the reaction mixture stirred at r.t. for 16 hours. The reaction mixture was combined with another batch (0.219 mmol), and concentrated to give a residue.

The crude residue was triturated with water-ethanol and the resulting precipitate collected by filtration, washed with ethyl acetate, diethyl ether and dried to give 246 mg (90% purity, 63% combined yield over two steps) of the desired product as pale yellow solid.

LC-MS (Method 5): Rt=0.85 min, MS (ESIpos): m/z=603 [M+H]⁺

¹H-NMR (400 MHz, DMSO-D₆) δ (ppm): 2.29 (s, 3H), 4.80 (d, 4H), 6.90 (s, 1H), 7.37 (d, 2H), 7.53-7.54 (m, 1H), 7.60-7.66 (m, 2H), 7.83-7.97 (m, 5H), 8.10 (s, 2H), 8.24 (s, 1H), 8.53-8.55 (m, 1H), 8.63-8.65 (m, 2H).

### Example 20

### N-{4-[5-(1H-indol-5-yl)-6-oxo-1,6-dihydropyridazin-3-yl]phenyl}-1,3-dihydro-2H-pyrrolo[3,4-c]pyridine-2-carboxamide

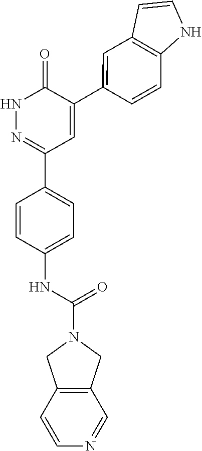 [SMILES: O=C(Nc1ccc(-c2cc(-c3ccc4[nH]ccc4c3)c(=O)[nH]n2)cc1)N1Cc2ccncc2C1]

A mixture of N-(4-{5-[1-(4-methylbenzene-1-sulfonyl)-1H-indol-5-yl]-6-oxo-1,6-dihydropyridazin-3-yl}phenyl)-1,3-dihydro-2H-pyrrolo[3,4-c]pyridine-2-carboxamide (50.0 mg, 83.0 μmol) in ethanol (10 mL) and NaOH (210 μl, 2.0 M, 410 μmol) was refluxed (80° C.) for 16 h. It was cooled to r.t. and acidified with 2 M HCl and basified again with saturated sodium carbonate solution. The mixture was concentrated under vacuum to remove the volatiles and the residue was diluted with water. The resulting solid was collected by filtration, washed with small amount of ethanol and dried to give the crude product. It was purified by preparative HPLC and then dried on the freeze drier to give 15.00 mg of the desired product (99% purity, 40% yield).

HPLC: Instrument: Waters mass directed auto-purification system, pump head/gradient module: 2545 Binary gradient module, fraction collector: 2767 Waters sample manager, 2998 Photodiode array detector, MassLynx software. Column: XSelect CSH C18, 19×150 mm, 5 μm; Eluent A: 0.1% formic acid in water; Eluent B: acetonitrile; gradient: 10-50% B over 10 mins; rate 20 mL/min, temperature 25° C.

LC-MS (Method 3): Rt=0.57 min; MS (ESIpos): m/z=449 [M+H]⁺

1H-NMR (400 MHz, DMSO-D6); δ (ppm)=4.74-4.81 (m, 4H), 6.50 (s, 1H), 7.37-7.44 (m, 3H), 7.67-7.74 (m, 3H), 7.86-7.88 (m, 2H), 8.05 (s, 1H), 8.30 (s, 1H), 8.46-8.48 (m, 1H), 8.59 (s, 2H), 11.22 (s, 1H)

### Example 21

### N-{4-[5-(1H-Indol-5-yl)-6-oxo-1,4,5,6-tetrahydropyridazin-3-yl]phenyl}-1,3-dihydro-2H-pyrrolo[3,4-c]pyridine-2-carboxamide

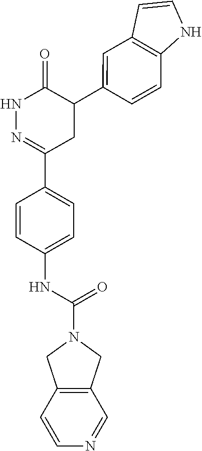 [SMILES: O=C1NN=C(c2ccc(NC(=O)N3Cc4ccncc4C3)cc2)CC1c1ccc2[nH]ccc2c1]

N-{4-[5-(1H-Indol-5-yl)-6-oxo-1,6-dihydropyridazin-3-yl]phenyl}-1,3-dihydro-2H-pyrrolo[3,4-c]pyridine-2-carboxamide (see Example 20, 10.0 mg, 0.022 mmol) was dissolved in acetic acid (2 mL). The mixture was heated with vigorous stirring at 90° C. in a preheated bath for 2 min. Zinc dust (7.2 mg, 0.11 mmol) was added and the mixture was heated at 90° C. The mixture was concentrated under vacuum and the residue was dissolved in DMSO and filtered. The filtrate was purified by preparative HPLC and followed by freeze drying afforded 2.5 mg of the desired product (100% purity, 25% yield) as a colourless solid.

HPLC: Instrument: Waters mass directed auto-purification system, pump head/gradient module: 2545 Binary gradient module, fraction collector: 2767 Waters sample manager, 2998 Photodiode array detector, MassLynx software. Column: XSelect CSH C18, 19×150 mm, 5 μm; Eluent A: 0.1% formic acid in water; Eluent B: acetonitrile; gradient: 16-20% over 10 mins; rate 20 mL/min, temperature 25° C.

LC-MS (Method 4): Rt=0.89 min; MS (ESIpos): m/z=451 [M+H]⁺

1H-NMR (400 MHz, DMSO-D6) δ 3.18-3.25 (m, 1H), 3.80 (t, 1H), 4.77 (br d, 4H), 6.31 (t, 1H), 6.96 (dd, 1H), 7.25-7.31 (m, 2H), 7.34 (s, 1H), 7.40 (d, 1H), 7.56-7.61 (m, 2H), 7.63-7.68 (m, 2H), 8.46 (d, 1H), 8.56 (d, 2H), 10.99 (s, 1H), 11.00 (br s, 1H);

### Intermediate 22-1

### N-{4-[5-{1-[Oxan-2-yl]-1H-indazol-5-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide

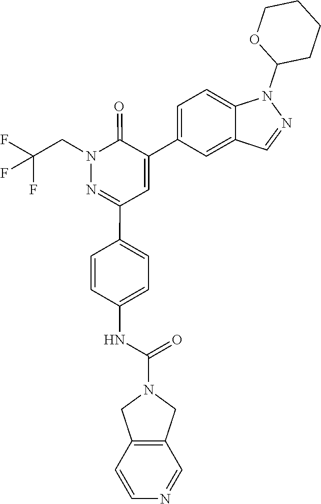 [SMILES: O=C(Nc1ccc(-c2cc(-c3ccc4c(cnn4C4CCCCO4)c3)c(=O)n(CC(F)(F)F)n2)cc1)N1Cc2ccncc2C1]

A mixture of N-(4-{6-oxo-5-[1-(tetrahydro-2H-pyran-2-yl)-1H-indazol-5-yl]-1,6-dihydropyridazin-3-yl}phenyl)-1,3-dihydro-2H-pyrrolo[3,4-c]pyridine-2-carboxamide, (see Intermediate 16-7, 500 mg 0.94 mmol), 2,2,2-trifluoroethyl trifluoromethanesulfonate (0.16 mL, 1.12 mmol), and potassium carbonate (259 mg, 1.87 mmol) in DMF (20 mL) was stirred at r.t. for 16 h. The mixture was poured into water and the resulting solid was collected by filtration to give 410 mg of the desired product (92% purity, 65% yield) as pale yellow solid.

LC-MS (Method 4): Rt=1.73 min; MS (ESIneg): m/z=(M−H)⁻ 614

¹H-NMR (400 MHz, CDCl₃) δ [ppm]: 1.68-1.83 (m, 3H), 2.08-2.19 (m, 2H), 2.52-2.61 (m, 1H), 3.74-3.79 (m, 1H), 4.02 (d, 1H), 4.89-5.00 (m, 6H), 5.76 (d, 1H), 6.42 (s, 1H), 7.30 (d, 1H), 7.59-7.61 (m, 2H, 7.66-7.69 (m, 1H), 7.80-7.85 (m, 4H), 8.09 (s, 1H), 8.34 (s, 1H), 8.57-8.59 (m, 1H), 8.63 (s, 1H).

### Example 22

### N-{4-[5-(1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide

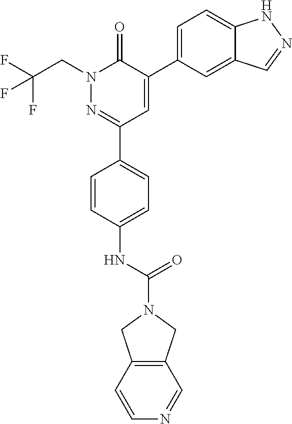 [SMILES: O=C(Nc1ccc(-c2cc(-c3ccc4[nH]ncc4c3)c(=O)n(CC(F)(F)F)n2)cc1)N1Cc2ccncc2C1]

A mixture of N-{4-[5-{1-[oxan-2-yl]-1H-indazol-5-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide (50.0 mg, 81.2 μmol) in 3 M HCl/CPME (5.0 mL) and water was stirred at r.t. for 2 h. The mixture was concentrated under vacuum and the residue was purified by preparative HPLC and followed by concentrated under vacuum afforded 9 mg of the desired product (99% purity, 21% yield) as a colourless solid.

HPLC: Instrument: Waters mass directed auto-purification system, pump head/gradient module: 2545 Binary gradient module, fraction collector: 2767 Waters sample manager, 2998 Photodiode array detector, MassLynx software. Column: XSelect CSH C18, 19×150 mm, 5 μm; Eluent A: 0.1% formic acid in water; Eluent B: acetonitrile; gradient: 20-40% over 10 mins; rate 20 mL/min, temperature 25° C.

LC-MS (Method 4): Rt=1.68 min; MS (ESipos): m/z=532 [M+H]⁺

¹H NMR (400 MHz, DMSO-d6) δ [ppm]: 4.81 (d, 4H), 5.07-5.11 (m, 2H), 7.36 (d, 1H), 7.58-7.60 (m, 1H), 7.70-7.72 (m, 2H), 7.89-7.91 (m, 3H), 8.17-8.18 (m, 2H), 8.45-8.47 (m, 2H), 8.58-8.64 (m, 2H).

### Intermediate 23-1

### N-{4-[5-{1-[Oxan-2-yl]-1H-indazol-4-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide

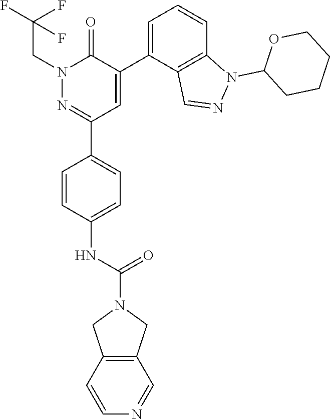 [SMILES: O=C(Nc1ccc(-c2cc(-c3cccc4c3cnn4C3CCCCO3)c(=O)n(CC(F)(F)F)n2)cc1)N1Cc2ccncc2C1]

A mixture of N-[4-(5-{1-[oxan-2-yl]-1H-indazol-4-yl}-6-oxo-1,6-dihydropyridazin-3-yl)phenyl]-1,3-dihydro-2H-pyrrolo[3,4-c]pyridine-2-carboxamide (see Intermediate 18-, 500 mg, 0.94 mmol), 2,2,2-trifluoroethyl trifluoromethanesulfonate (0.16 mL, 1.12 mmol) and potassium carbonate (259 mg, 1.87 mmol) in DMF (20 mL) was stirred at r.t. for 16 h. This mixture was poured into water and the resulting solid collected by filtration. The crude residue was purified by column chromatography (Biotage Isolera, 30 g; ZIP Sphere Silica Biotage cartridge, ethyl acetate/methanol gradient) to give the desired product, 235 mg. This was further purified by preparative HPLC to give 120 mg (99% purity, 20% yield) of the desired product.

HPLC: Instrument: Waters UV directed auto-purification system, pump head/gradient module 2545 Binary gradient module, fraction collector: 2767 Waters sample manager, 2996 Photodiode array detector, MassLynx software. Column: X-Bridge C18, 19×150 mm, 5 μm Eluent A: 0.1% ammonia in water; Eluent B: acetonitrile; gradient: 30-60% B over 10 mins; rate 20 mL/min, temperature 25° C.

LC-MS (Method 4): Rt=1.62 min; MS (ESIneg): m/z=614 [M−H]⁻

1H NMR (400 MHz, CHLOROFORM-D): δ (ppm)=1.67-1.83 (m, 3H), 2.03-2.20 (m, 2H), 2.57-2.62 (m, 1H), 3.73-3.78 (m, 1H), 4.0-4.07 (m, 1H), 4.88-5.02 (m, 6H), 5.76-5.78 (m, 1H), 6.45 (s, 1H), 7.29 (d, 1H), 7.45-7.48 (m, 1H), 7.58-7.60 (m, 3H), 7.69-7.72 (m, 1H), 7.78-7.81 (m, 2H), 7.94 (s, 1H), 8.06 (s, 1H), 8.57-8.58 (m, 1H), 8.62 (s, 1H).

### Example 23

### N-{4-[5-(1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide

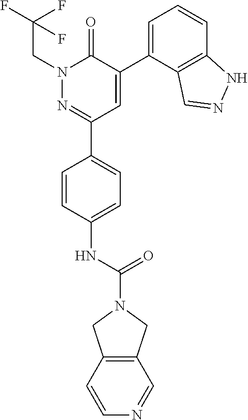 [SMILES: O=C(Nc1ccc(-c2cc(-c3cccc4[nH]ncc34)c(=O)n(CC(F)(F)F)n2)cc1)N1Cc2ccncc2C1]

A mixture of N-{4-[5-{1-[oxan-2-yl]-1H-indazol-4-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide (120 mg, 0.19 mmol) in hydrogen chloride (4M in 1,4-dioxane, 5 mL) and water, 0.5 mL, was stirred at r.t. for 2 hours. The crude mixture was combined with another batch starting from N-{4-[5-{1-[oxan-2-yl]-1H-indazol-4-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide (20 mg, 32.5 μmol) and the solvent decanted. The residue was treated with saturated aqueous sodium bicarbonate solution and the resulting solid collected by filtration and dried to give the desired product, 100 mg (93% purity, 89% combined yield).

LC-MS (Method 4): Rt=1.75 min: MS (ESIpos): m/z=532 [M+H]⁺

¹H-NMR (400 MHz, DMSO-D6) δ [ppm]: 4.97 (d, 4H), 5.11 (q, 2H), 7.42-7.48 (m, 2H), 7.65 (d, 1H), 7.71 (d, 2H), 7.89 (d, 2H), 7.95-7.99 (m, 1H), 8.02 (br s, 1H), 8.19 (s, 1H), 8.76 (d, 1H), 8.82 (s, 1H), 8.90 (s, 1H).

### Intermediate 24-1

### 7-(4,4,5,5-tetramethyl-1,3,2-dioxaborolan-2-yl)quinoline

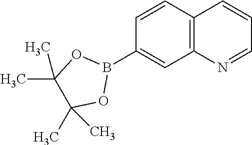 [SMILES: CC1(C)OB(c2ccc3cccnc3c2)OC1(C)C]

A mixture of 7-bromoquinoline (CAS 4965-36-0, 2 g, 9.60 mmol), 4,4,5,5-tetramethyl-1,3,2-dioxaborolan-2-yl-4′,4′,5′,5′-tetramethyl-1,3,2-dioxaborolane (2.68 g, 10.5 mmol), potassium acetate (2.83 g, 28.8 mmol) and bis(diphenylphosphino)ferrocene]dichloropalladium (351 mg, 0.48 mmol) in dry dioxane (50 mL) was degassed under argon. The mixture was heated at 110° C. for 5 h. This mixture was filtered through celite, washed with ethyl acetate and the filtrate was concentrated under vacuum to give 4.2 g of the crude product (50% pure by 1H NMR). 2.9 g of the crude product was purified by using isolera (80 g column, eluent: heptane-ethyl acetate, 1:9, 5CV, 10-60% gradient 10CV) to give 1.5 g of the desired product (95% purity, 58% yield).

LC-MS (Method 5): Rt=0.69 min; MS (ESIpos): m/z=256 [M+H]⁺

1H NMR (400 MHz, CDCl3) [ppm]: 1.37 (s, 12H), 7.39-7.42 (m, 1H), 7.79 (d, 1H), 7.88 (d, 1H), 8.15 (d, 1H), 8.61 (s, 1H), 8.92 (d, 1H).

### Intermediate 24-2

### 6-(4-Aminophenyl)-4-(quinolin-7-yl)pyridazin-3(2H)-one

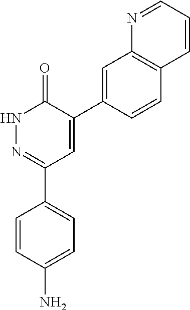 [SMILES: Nc1ccc(-c2cc(-c3ccc4cccnc4c3)c(=O)[nH]n2)cc1]

A mixture of 4-bromo-6-chloropyridazin-3(2H)-one, (Intermediate 16-3, 1.00 g, 4.77 mmol), 7-(4,4,5,5-tetramethyl-1,3,2-dioxaborolan-2-yl)quinoline (1.22 g, 4.77 mmol), and potassium carbonate (989 mg, 7.16 mmol), in 1,4-dioxane (75.0 mL) and water (15.0 mL) was degassed with argon for 10 minutes. Bis(diphenylphosphino)ferrocene]dichloropalladium(II) (174 mg, 0.24 mmol) was added and the mixture heated at 90° C. for 7 hours. 4-(4,4,5,5-Tetramethyl-1,3,2-dioxaborolan-2-yl)aniline (1.25 g 5.73 mmol), and bis(diphenylphosphino)ferrocene] dichloropalladium(II), 175 mg (0.24 mmol) were added. The mixture was heated at 100° C. for 16 hours. The mixture was cooled to r.t. The solid precipitated out was collected by filtration, washed with ethyl acetate and dried to give the desired product, 0.93 g (95% purity, 59% yield). The filtrate was concentrated and the residue was triturated with ethyl acetate-methanol to give the desired product, 0.75 g (67% purity, 33% yield).

LC-MS (Method 3): Rt=0.34 min; MS (ESIpos): m/z=315 [M+H]⁺

¹H NMR (400 MHz, DMSO-d₆) δ [ppm]: 5.45 (br s, 2H), 6.62 (d, 2H), 7.56 (q, 1H), 7.68 (d, 2H), 8.03 (d, 1H), 8.12-8.18 (m, 2H), 8.39 (d, 1H), 8.70 (s, 1H), 8.94 (d, 1H);

### Example 24

### N-{4-[6-Oxo-5-(quinolin-7-yl)-1,6-dihydropyridazin-3-yl]phenyl}-1,3-dihydro-2H-pyrrolo[3,4-c]pyridine-2-carboxamide

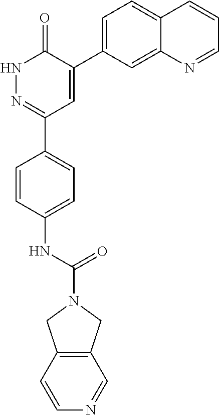 [SMILES: O=C(Nc1ccc(-c2cc(-c3ccc4cccnc4c3)c(=O)[nH]n2)cc1)N1Cc2ccncc2C1]

To a solution of 6-(4-aminophenyl)-4-(quinolin-7-yl)pyridazin-3(2H)-one, (800 mg, 2.54 mmol), in tetrahydrofuran, 160 mL, was added 4-nitrophenyl carbonochloridate (615 mg, 3.05 mmol). The reaction mixture was heated at 60° C. for 5 hours and concentrated under vacuum. The residue was dissolved in N,N-dimethylformamide, 20 mL and added to 2,3-dihydro-1H-pyrrolo[3,4-c]pyridine dihydrochloride (588 mg, 2.54 mmol) and N,N-diisopropylethylamine, (2.21 mL, 12.7 mmol) in dichloromethane (60 mL) and stirred at r.t. for 16 hours. The precipitate was collected by filtration, washed with water and dried to give the desired product, 850 mg (98% purity, 65% yield over two steps).

Products:

LC-MS (Method 3): Rt=0.32 min; MS (ESIpos): m/z=461 [M+H]⁺

1H-NMR (400 MHz, DMSO-D6) δ [ppm]: 4.79-4.81 (m, 4H), 7.41 (d, 1H), 7.54-7.57 (m, 1H), 7.68-7.71 (m, 2H), 7.91-7.93 (m, 2H), 8.03-8.05 (m, 1H), 8.15-8.17 (m, 1H), 8.31 (s, 1H), 8.38-8.40 (m, 1H), 8.46-8.48 (m, 1H), 8.58-8.60 (m, 2H), 8.74 (s, 1H), 8.93-8.94 (m, 1H).

### Intermediate 25-1

### Tert-Butyl {4-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]butyl}carbamate

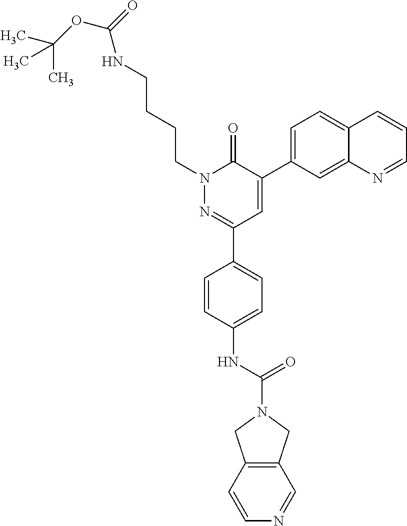 [SMILES: CC(C)(C)OC(=O)NCCCCn1nc(-c2ccc(NC(=O)N3Cc4ccncc4C3)cc2)cc(-c2ccc3cccnc3c2)c1=O]

A mixture of N-{4-[6-oxo-5-(quinolin-7-yl)-1,6-dihydropyridazin-3-yl]phenyl}-1,3-dihydro-2H-pyrrolo[3,4-c]pyridine-2-carboxamide (see Example 24, 278 mg, 604 μmol), 2-(4-bromobutyl)-1H-isoindole-1,3(2H)-dione (278 mg, 0.60 mmol) and potassium carbonate (166 mg, 1.2 mmol) in DMF (32 mL) was stirred at r.t. for 16 h. Then the mixture was heated at 50° C. for 6 h. The reaction mixture was poured into water (50 mL) and stirred for 1 h. The resulting solid was collected by filtration and purified by Isolera (10 g zipspere, eluent 5% methanol in DCM, 3 CV, 5-20% 10 CV) to give 100 mg of the desired product (97% purity, 25% yield).

LC-MS (Method 3): Rt=0.48 min; MS (ESIneg): m/z=630 [M−H]⁻

¹H-NMR (400 MHz, DMSO-D6): δ (ppm)=1.33 (s, 9H), 1.43-1.47 (m, 2H), 1.78-1.80 (m, 2H), 2.85-2.97 (m, 2H), 4.22-4.24 (m, 2H), 4.79-4.81 (m, 4H), 6.82 (s, 1H), 7.40-7.41 (m, 1H), 7.56 (q, 1H), 7.69-7.71 (m, 2H), 7.93-7.94 (m, 2H), 8.03-8.05 (m, 1H), 8.10-8.12 (m, 1H), 8.28 (s, 1H), 8.38-8.40 (m, 1H), 8.46-8.48 (m, 1H), 8.59-8.74 (m, 3H), 8.93-8.94 (m, 1H).

### Example 25

### N-{4-[1-(4-Aminobutyl)-6-oxo-5-(quinolin-7-yl)-1,6-dihydropyridazin-3-yl]phenyl}-1,3-dihydro-2H-pyrrolo[3,4-c]pyridine-2-carboxamide

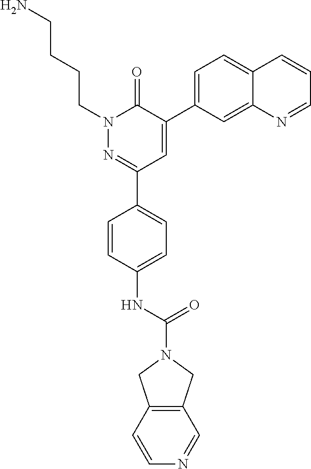 [SMILES: NCCCCn1nc(-c2ccc(NC(=O)N3Cc4ccncc4C3)cc2)cc(-c2ccc3cccnc3c2)c1=O]

Tert-butyl {4-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]butyl}carbamate (96.0 mg, 152 μmol) was stirred in dichloromethane (4.0 mL, 62 mmol) together with trifluoroacetic acid (180 μl, 2.3 mmol) for 1 day. The mixture was concentrated under reduced pressure and purified by preparative HPLC to give 64.8 mg of the title compound (95% purity, 76% yield).

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-6 min 10-50% B, rate 150 mL/min, temperature 25° C.

LC-MS (Method 2): Rt=0.93 min; MS (ESIpos): m/z=532 [M+H]⁺

¹H-NMR (400 MHz, DMSO-d₆) δ [ppm]: 1.54-1.65 (m, 2H), 1.84-1.96 (m, 2H), 2.79 (br t, 2H), 3.47-3.54 (m, 2H), 4.29 (br t, 2H), 4.84 (br d, 4H), 7.45 (d, 1H), 7.60 (dd, 1H), 7.75 (d, 2H), 7.94-8.02 (m, 2H), 8.08 (d, 1H), 8.12-8.20 (m, 1H), 8.34 (s, 1H), 8.40-8.47 (m, 2H), 8.51 (d, 1H), 8.63 (s, 1H), 8.70 (br d, 2H), 8.98 (dd, 1H).

### Intermediate 26-1

### 4-bromo-1-(4-methylbenzene-1-sulfonyl)-1H-benzimidazole

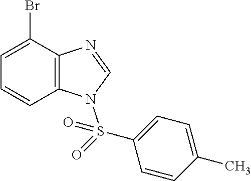 [SMILES: Cc1ccc(S(=O)(=O)n2cnc3c(Br)cccc32)cc1]

4-Bromo-1H-benzimidazole (CAS 83741-35-9, 1.0 g, 5.07 mmol) was added to a round bottom flask, to which was added tetrahydrofuran (50 mL) under argon. Sodium hydride (60% dispersion in mineral oil, 304 mg, 7.6 mmol) was added and stirred for 30 min. The reaction mixture was then cooled to 0° C. and tosyl chloride (1.16 g, 6.10 mmol) was added. The mixture was allowed to warm to r.t. and stirred for 16 h. After completion the crude mixture was quenched with saturated ammonium chloride solution and concentrated under vacuum. The residue was partitioned between water and ethyl acetate. The ethyl acetate layer was collected, dried over sodium sulfate and concentrated. The crude product was purified by Isolera (30 g, eluent: heptane/ethyl acetate, gradient) to give 1.0 g of the desired product (99% purity, 55% yield) as pale brown solid.

LC-MS (Method 5): Rt=0.98 min; MS (ESpos): m/z=351 [M+H]⁺, 353 [M+H]⁺

1H-NMR (400 MHz, CHLOROFORM-D): δ [ppm]: 2.39 (s, 3H), 7.23-7.32 (m, 3H), 7.53-7.55 (m, 1H), 7.80-7.87 (m, 3H), 8.43 (s, 1H).

### Intermediate 26-2

### 1-(4-Methylbenzene-1-sulfonyl)-4-(4,4,5,5-tetramethyl-1,3,2-dioxaborolan-2-yl)-1H-benzimidazole

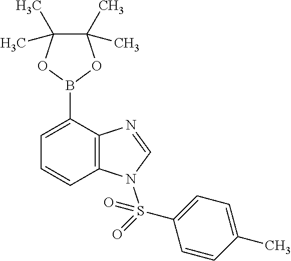

A mixture of 4-bromo-1-(4-methylbenzene-1-sulfonyl)-1H-benzimidazole (800 mg, 2.27 mmol), 4,4,5,5-tetramethyl-1,3,2-dioxaborolan-2-yl-4′,4′,5′,5′-tetramethyl-1,3,2-dioxaborolane (636 mg, 2.50 mmol), potassium acetate (670 mg, 6.83 mmol) and bis(diphenylphosphino)ferrocene]dichloropalladium (83.3 mg, 0.11 mmol) in dry dioxane (40 mL) was degassed under argon for 10 min. The mixture was heated at 100° C. for 16 h. This reaction mixture was purified by Isolera (45 g, zip sphere cartridge) (eluent: heptane-ethyl acetate, gradient) to give 900 mg of the desired product (80% purity, 80% yield).

LC-MS (Method 5): Rt=0.81 min; MS (ESipos): m/z=317 [M+H]⁺, (pinacol ester fragmention).

1H-NMR (400 MHz, CHLOROFORM-D): δ [ppm] 1.42 (s, 12H), 2.37 (s, 3H), 7.25-7.27 (m, 2H), 7.35-7.39 (m, 1H), 7.78-7.82 (m, 3H), 7.94-7.96 (m, 1H), 8.47 (s, 1H).

### Intermediate 26-3

### 6-Chloro-4-[1-(4-methylbenzene-1-sulfonyl)-1H-benzimidazol-4-yl]pyridazin-3(2H)-one

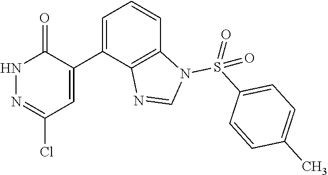 [SMILES: Cc1ccc(S(=O)(=O)n2cnc3c(-c4cc(Cl)n[nH]c4=O)cccc32)cc1]

A mixture of 4-bromo-6-chloropyridazin-3(2H)-one (see Intermediate 16-3, 340 mg, 1.62 mmol), 1-[(4-methylphenyl)sulfonyl]-4-(4,4,5,5-tetramethyl-1,3,2-dioxaborolan-2-yl)-1H-benzimidazole (see Intermediate 26-2, 800 mg, 1.62 mmol), potassium carbonate (337 mg, 2.44 mmol) were suspended in dioxane/water (5:1, 42 mL) and the mixture was degassed for 10 min. Bis(diphenylphosphino)ferrocene]dichloropalladium (59 mg, 0.081 mmol) was added and the mixture was heated to 90° C. for 16 h. The mixture was cooled to r.t. and the solid was collected by filtration, washed with water and ethyl acetate to give 200 mg of the desired product (97% purity, 30% yield) as colourless solid.

LC-MS (Method 3): Rt=0.87 min; MS (ESIpos): m/z=401 [M+H]⁺

1H-NMR (300 MHz, DMSO-D6) δ (ppm): 2.28 (s, 3H), 7.43-7.51 (m, 3H), 7.95-8.14 (m, 5H), 8.95 (br s, 1H).

### Intermediate 26-4

### 6-(4-Aminophenyl)-4-[1-(4-methylbenzene-1-sulfonyl)-1H-benzimidazol-4-yl]pyridazin-3(2H)-one

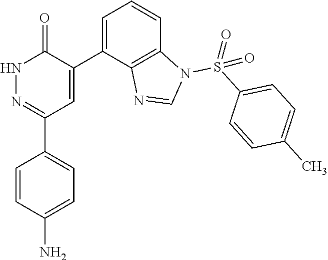 [SMILES: Cc1ccc(S(=O)(=O)n2cnc3c(-c4cc(-c5ccc(N)cc5)n[nH]c4=O)cccc32)cc1]

A mixture of 6-chloro-4-[1-(4-methylbenzene-1-sulfonyl)-1H-benzimidazol-4-yl]pyridazin-3(2H)-one (240 mg, 0.60 mmol), 4-(4,4,5,5-tetramethyl-1,3,2-dioxaborolan-2-yl)aniline (157 mg, 0.72 mmol), potassium carbonate (165 mg, 1.19 mmol) were suspended in dioxane/water (5:1, 48 mL). The mixture was degassed for 10 min and bis(diphenylphosphino)ferrocene]-dichloropalladium (22 mg, 0.03 mmol) was added and heated at 100° C. for 16 h. The mixture was cooled to r.t. and partitioned between ethyl acetate and water, the resulting solid was collected by filtration, washed with methanol to give 140 mg of the desired product (95% purity, 46% yield) as brown solid.

LC-MS (Method 5): Rt=0.82 min; MS (ESIpos): m/z=458 [M+H]⁺

1H-NMR (400 MHz, DMSO-d6) δ [ppm]: 2.31 (s, 3H), 5.44 (br s, 2H), 6.57-6.59 (m, 2H), 7.44-7.51 (m, 6H), 7.92-7.94 (m, 1H), 7.98-8.0 (m, 1H), 8.05-8.07 (m, 2H), 8.42 (s, 1H), 8.90 9s, 1H).

### Intermediate 26-5

### 4-nitrophenyl (4-{5-[1-(4-methylbenzene-1-sulfonyl)-1H-benzimidazol-4-yl]-6-oxo-1,6-dihydropyridazin-3-yl}phenyl)carbamate

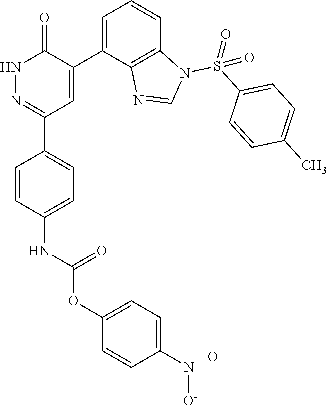 [SMILES: Cc1ccc(S(=O)(=O)n2cnc3c(-c4cc(-c5ccc(NC(=O)Oc6ccc(N[O-])cc6)cc5)n[nH]c4=O)cccc32)cc1 ; O]

6-(4-Aminophenyl)-4-[1-(4-methylbenzene-1-sulfonyl)-1H-benzimidazol-4-yl]pyridazin-3(2H)-one (140 mg, 0.31 mmol) was taken in a mixture of dichloromethane (10 mL) and DMF (2 mL) at r.t. under argon. Pyridine (49 μl, 0.61 mmol) was added followed by 4-nitrophenyl carbonochloridate (74 mg, 0.36 mmol). The mixture was stirred at r.t. for 4 h. More 4-nitrophenyl carbonochloridate (0.37 mmol) was added and the mixture was stirred for another 2 h. This mixture was used directly in the experiment of Intermediate 26-6.

### Intermediate 26-6

### N-(4-{5-[1-(4-Methylbenzene-1-sulfonyl)-1H-benzimidazol-4-yl]-6-oxo-1,6-dihydropyridazin-3-yl}phenyl)-1,3-dihydro-2H-pyrrolo[3,4-c]pyridine-2-carboxamide

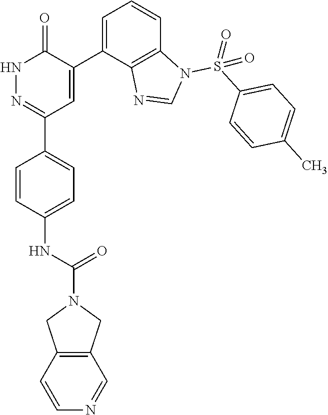 [SMILES: Cc1ccc(S(=O)(=O)n2cnc3c(-c4cc(-c5ccc(NC(=O)N6Cc7ccncc7C6)cc5)n[nH]c4=O)cccc32)cc1]

N,N-Diisopropylethylamine (0.27 mL, 1.53 mmol) followed by 2,3-dihydro-1H-pyrrolo[3,4-c]pyridine hydrochloride (118 mg, 0.61 mmol) was added to the reaction mixture from experiment of Intermediate 26-5 at r.t. under argon. The mixture was stirred at r.t. for 16 h. The mixture was concentrated under vacuum and the crude residue was triturated with a mixture of water and ethanol. The resulting solid was collected by filtration, washed with dichloromethane and dried to give 140 mg of the desired product (63% purity, 48% yield). This was purified by preparative HPLC and then followed by concentrated under vacuum to afforded 40 mg of the desired product (99% purity, 21% yield) as a colourless solid.

HPLC: Instrument: Waters mass directed auto-purification system, pump head/gradient module: 2545 Binary gradient module, fraction collector: 2767 Waters sample manager, 2998 Photodiode array detector, MassLynx software. Column: XSelect CSH C18, 19×150 mm, 5 μm; Eluent A: 0.1% ammonia in water; Eluent B: acetonitrile; gradient: 30-70% over 10 mins; rate 20 mL/min, temperature 25° C.

LC-MS (Method 4): Rt=1.44 min; MS (ESIpos): m/z=604 [M+H]⁺

¹H-NMR (301 MHz, DMSO-D6) δ [ppm]: 2.34 (s, 3H), 4.81 (d, 4H), 7.41-7.54 (m, 4H), 7.66-7.77 (m, 4H), 7.94-8.03 (m, 2H), 8.08 (d, 2H), 8.48 (d, 1H), 8.53 (s, 1H), 8.59-8.60 (m, 2H), 8.93 (s, 1H).

### Example 26

### N-{4-[5-(1H-Benzimidazol-4-yl)-6-oxo-1,6-dihydropyridazin-3-yl]phenyl}-1,3-dihydro-2H-pyrrolo[3,4-c]pyridine-2-carboxamide

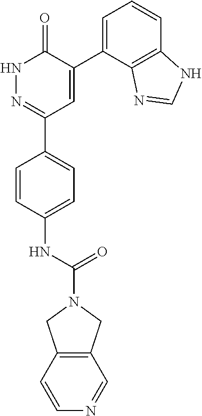 [SMILES: O=C(Nc1ccc(-c2cc(-c3cccc4[nH]cnc34)c(=O)[nH]n2)cc1)N1Cc2ccncc2C1]

A mixture of N-(4-{5-[1-(4-methylbenzene-1-sulfonyl)-1H-benzimidazol-4-yl]-6-oxo-1,6-dihydropyridazin-3-yl}phenyl)-1,3-dihydro-2H-pyrrolo[3,4-c]pyridine-2-carboxamide (30 mg, 0.05 mmol) in ethanol (5 mL) and aqueous sodium hydroxide solution (2M, 0.12 mL, 0.25 mmol) was heated at 60° C. for 16 h. The reaction mixture was cooled to r.t. and acidified with aqueous hydrochloric acid (2M) and basified again with saturated sodium carbonate solution. The resulting solid was collected by filtration, washed with small amount of ethanol, dried to give 45 mg of the desired product. This was purified by preparative HPLC and concentrated under vacuum to afforded 8 mg (95% purity, 34% yield) of the desired product as a colourless solid.

HPLC: Instrument: Waters mass directed auto-purification system, pump head/gradient module: 2545 Binary gradient module, fraction collector: 2767 Waters sample manager, 2998 Photodiode array detector, MassLynx software. Column: XSelect CSH C18, 19×150 mm, 5 μm; Eluent A: 0.1% ammonia in water; Eluent B: acetonitrile; gradient: 10-50% over 10 mins; rate 20 mL/min, temperature 25° C.

LC-MS (Method 6): Rt=1.21 min; MS (ESIpos): m/z=450 [M+H]⁺

1H-NMR (400 MHz, DMSO-D6) δ [ppm]: 4.79-4.81 (m, 4H), 7.26 (t, 1H), 7.41 (d, 1H), 7.65-7.70 (m, 3H), 7.81 (d, 2H), 8.27 (s, 1H), 8.47 (d, 1H), 8.59 (d, 2H).

### Intermediate 27-1

### Tert-Butyl [(2S)-1-({4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}amino)-1-oxopropan-2-yl]carbamate

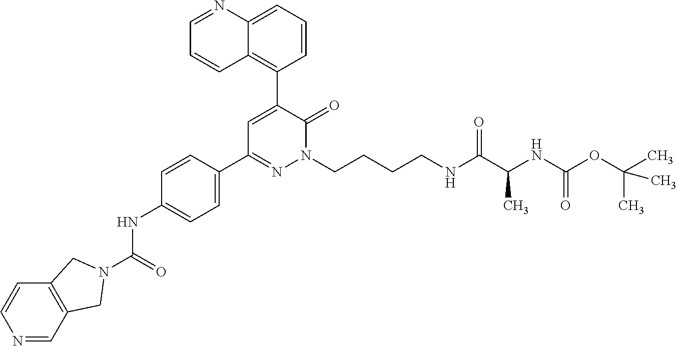 [SMILES: C[C@H](NC(=O)OC(C)(C)C)C(=O)NCCCCn1nc(-c2ccc(NC(=O)N3Cc4ccncc4C3)cc2)cc(-c2cccc3ncccc23)c1=O]

To a mixture of N-(tert-butoxycarbonyl)-L-alanine (27.2 mg, 144 μmol), 4-methylmorpholin (28 μl, 250 μmol) and HATU (47.9 mg, 126 μmol) in DMF (0.65 mL) was added N-{4-[1-(4-aminobutyl)-6-oxo-5-(quinolin-5-yl)-1,6-dihydropyridazin-3-yl]phenyl}-1,3-dihydro-2H-pyrrolo [3,4-c]pyridine-2-carboxamide (see Example 5, 50.0 mg, 94.1 μmol) after 15 min of stirring. Stirring was continued at r.t. for 3 h. Then the reaction mixture was diluted with formic acid (9.6 μl, 250 μmol) in water (0.1 mL) and DMSO and purified by preparative HPLC to give 46.3 mg (90% purity, 63% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm; Eluent A: water+0.1% formic acid; Eluent B: acetonitrile; gradient: 0-6 min 5-7% B, 6-12 min 7-25% B, 12-14 min 25% B; rate 150 mL/min, temperature 25° C.

LC-MS (Method 2): Rₜ=0.99 min; MS (ESIpos): m/z=703 [M+H]⁺

¹H-NMR (400 MHz, DMSO-d6) δ [ppm]: 1.132 (3.31), 1.150 (3.22), 1.341 (16.00), 1.490 (0.71), 1.508 (0.98), 1.527 (0.80), 1.826 (0.76), 1.845 (0.98), 1.863 (0.69), 3.102 (0.53), 3.117 (0.53), 3.146 (0.56), 3.162 (0.58), 3.896 (0.64), 4.229 (1.07), 4.816 (2.60), 4.835 (2.60), 6.822 (0.76), 6.840 (0.73), 7.433 (1.40), 7.445 (1.42), 7.505 (1.33), 7.516 (1.31), 7.526 (1.33), 7.537 (1.31), 7.667 (1.36), 7.669 (1.38), 7.685 (1.67), 7.687 (1.58), 7.700 (3.02), 7.723 (3.38), 7.801 (0.84), 7.814 (0.51), 7.836 (1.42), 7.854 (1.33), 7.858 (1.64), 7.875 (1.27), 7.902 (3.42), 7.924 (2.64), 8.071 (1.22), 8.092 (1.13), 8.117 (1.76), 8.139 (1.49), 8.153 (4.33), 8.496 (1.69), 8.508 (1.58), 8.613 (2.56), 8.645 (2.44), 8.934 (1.49), 8.938 (1.58), 8.944 (1.51), 8.948 (1.33).

### Intermediate 27-2

### [(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]ammonium Trifluoroacetate

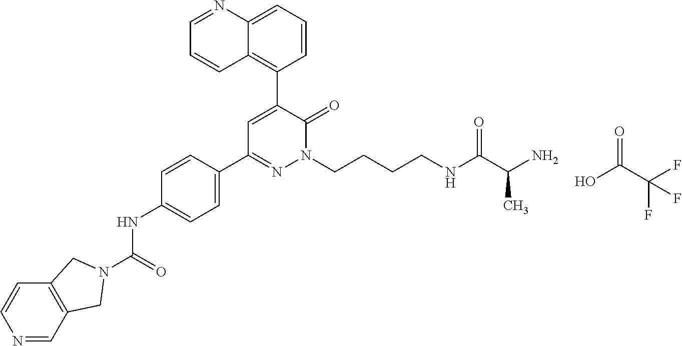 [SMILES: C[C@H](N)C(=O)NCCCCn1nc(-c2ccc(NC(=O)N3Cc4ccncc4C3)cc2)cc(-c2cccc3ncccc23)c1=O ; O=C(O)C(F)(F)F]

A mixture of tert-butyl [(2S)-1-({4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}amino)-1-oxopropan-2-yl]carbamate (43.7 mg, 62.2 μmol) and trifluoroacetic acid (130 μl, 1.7 mmol) in dichloromethane (0.88 mL) was stirred at r.t. for 2 h. Then the mixture was diluted with toluene, concentrated under reduced pressure to give 65.0 mg (66% purity, 96% yield) of the title compound

LC-MS (Method 1): Rₜ=0.59 min, MS (ESIneg): m/z=715 [M−H]⁻

¹H-NMR (400 MHz, DMSO-d6) δ [ppm]: 1.309 (2.95), 1.326 (2.95), 1.442 (0.62), 1.460 (0.60), 1.531 (1.40), 1.556 (0.65), 1.576 (0.59), 1.860 (0.57), 1.881 (0.70), 1.898 (0.49), 2.083 (16.00), 2.297 (13.94), 2.321 (1.63), 2.327 (1.03), 2.332 (0.70), 2.441 (1.14), 3.204 (0.52), 3.219 (0.52), 3.766 (0.49), 4.261 (1.06), 4.366 (1.38), 4.382 (1.71), 4.398 (1.62), 4.524 (2.29), 4.933 (2.03), 4.975 (1.94), 5.757 (0.70), 7.124 (0.54), 7.142 (1.43), 7.161 (2.86), 7.162 (3.06), 7.180 (3.54), 7.185 (2.11), 7.230 (3.06), 7.235 (1.02), 7.249 (3.19), 7.263 (0.56), 7.267 (1.24), 7.607 (0.73), 7.618 (0.71), 7.629 (0.75), 7.639 (0.71), 7.705 (2.14), 7.727 (2.40), 7.739 (1.00), 7.756 (1.08), 7.881 (0.75), 7.896 (0.78), 7.922 (2.78), 7.941 (1.94), 7.944 (2.17), 7.958 (0.89), 8.031 (1.00), 8.175 (1.24), 8.202 (3.48), 8.239 (0.76), 8.260 (0.71), 8.375 (0.68), 8.747 (1.08), 8.762 (1.32), 8.768 (1.79), 8.877 (1.63), 9.028 (0.97), 9.032 (0.98), 9.039 (0.98), 9.043 (0.87).

### Intermediate 27-3

### N-(tert-Butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide

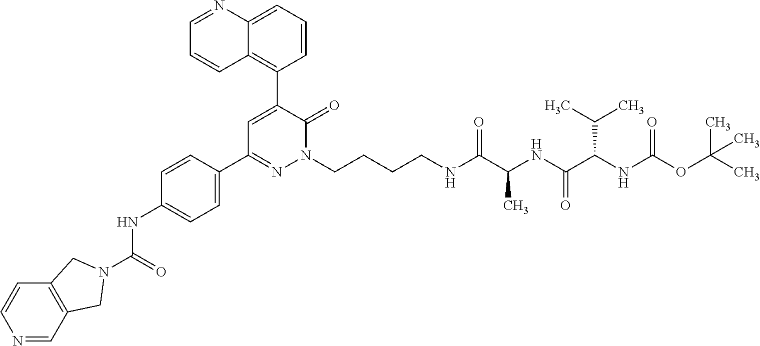 [SMILES: CC(C)[C@H](NC(=O)OC(C)(C)C)C(=O)N[C@@H](C)C(=O)NCCCCn1nc(-c2ccc(NC(=O)N3Cc4ccncc4C3)cc2)cc(-c2cccc3ncccc23)c1=O]

To a mixture of N-(tert-butoxycarbonyl)-L-valine (19.9 mg, 91.6 μmol), 4-methylmorpholin (16 μl. 150 μmol) and HATU (30.7 mg, 80.8 μmol) in DMF (0.3 mL) was added raw [(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]ammonium trifluoroacetate (see Intermediate 27-2, 65.0 mg, 66% purity, 59.9 μmol) in 4-methylmorpholin (10 μl, 100 μmol) and DMF (0.39 mL) after 20 min of stirring. Stirring was continued at r.t. for 6 h. Then the reaction mixture was diluted with formic acid (9.0 μl, 240 μmol) in water (0.1 mL) and DMSO and purified by preparative HPLC to give 27.0 mg (90% purity, 51% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-8 min 7% B, 8-20 min 7-25% B, 20-25 min 25% B; rate 150 mL/min, temperature 25° C.

LC-MS (Method 2): Rₜ=1.04 min; MS (ESIpos): m/z=802 [M+H]⁺

¹H-NMR (400 MHz, DMSO-d6) δ [ppm]: 0.763 (2.45), 0.780 (2.65), 0.806 (3.47), 0.823 (3.45), 1.168 (4.06), 1.185 (4.08), 1.359 (16.00), 1.488 (0.61), 1.509 (0.87), 1.527 (0.73), 1.825 (0.73), 1.844 (0.92), 1.862 (0.69), 2.071 (2.50), 2.518 (0.85), 2.522 (0.56), 2.539 (3.42), 3.107 (0.50), 3.124 (0.53), 3.143 (0.56), 3.159 (0.56), 3.769 (0.56), 3.774 (0.56), 4.233 (1.14), 4.251 (1.03), 4.268 (0.56), 4.817 (2.22), 4.835 (2.23), 6.732 (0.68), 6.755 (0.65), 7.431 (1.24), 7.445 (1.27), 7.504 (1.31), 7.514 (1.22), 7.525 (1.30), 7.535 (1.24), 7.667 (1.26), 7.669 (1.35), 7.684 (1.53), 7.687 (1.49), 7.703 (2.59), 7.725 (2.97), 7.836 (1.60), 7.843 (0.81), 7.853 (1.51), 7.856 (1.92), 7.875 (1.25), 7.901 (3.26), 7.924 (3.13), 8.074 (1.11), 8.076 (1.12), 8.078 (0.99), 8.096 (1.04), 8.117 (1.63), 8.139 (1.37), 8.156 (4.60), 8.496 (1.81), 8.508 (1.67), 8.613 (2.53), 8.644 (2.04), 8.934 (1.46), 8.938 (1.49), 8.945 (1.42), 8.949 (1.29).

### Intermediate 27-4

### [(1S)-1-[[(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]carbamoyl]-2-methyl-propyl]ammonium Trifluoroacetate

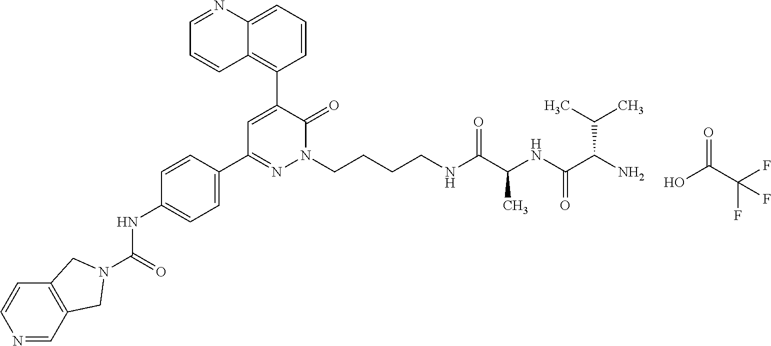 [SMILES: CC(C)[C@H](N)C(=O)N[C@@H](C)C(=O)NCCCCn1nc(-c2ccc(NC(=O)N3Cc4ccncc4C3)cc2)cc(-c2cccc3ncccc23)c1=O ; O=C(O)C(F)(F)F]

A mixture of N-(tert-butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide (27.0 mg, 33.7 μmol) and trifluoroacetic acid (70 μl, 910 μmol) in dichloromethane (0.48 mL) was stirred at r.t. for 2 h. Then the mixture was diluted with toluene, concentrated under reduced pressure (azeotropic distillation with toluene was executed two times) to give 26.0 mg (95% yield) of the title compound.

LC-MS (Method 1): Rt=0.62 min; MS (ESIneg): m/z=700 [M−H]⁻

¹H-NMR (400 MHz, DMSO-d6) δ [ppm]: 0.89 (d, 6H), 1.22 (d, 3H), 1.47-1.56 (m, 2H), 1.81-1.89 (m, 2H), 1.97-2.05 (m, 1H), 3.08-3.21 (m, 2H), 3.58 (br t, 1H), 4.24 (br s, 2H), 4.28-4.45 (m, 2H), 4.92 (br d, 4H), 7.58 (dd, 1H), 7.69-7.74 (m, 2H), 7.76 (br d, 1H), 7.88-7.96 (m, 3H), 7.98-8.10 (m, 3H), 8.13-8.22 (m, 2H), 8.54 (d, 1H), 8.69 (d, 1H), 8.73 (s, 1H), 8.81 (s, 1H), 9.00 (dd, 1H).

### Final Intermediate 27-5

### N-[6-(2,5-Dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide

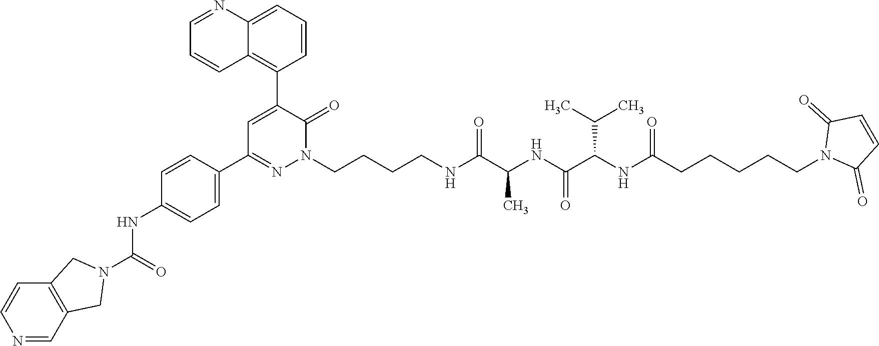 [SMILES: CC(C)[C@H](NC(=O)CCCCCN1C(=O)C=CC1=O)C(=O)N[C@@H](C)C(=O)NCCCCn1nc(-c2ccc(NC(=O)N3Cc4ccncc4C3)cc2)cc(-c2cccc3ncccc23)c1=O]

A mixture of [(1S)-1-[[(1S)-2-[4-[3-[4-(1,3-dihydropyrrolo[3,4-c]pyridine-2-carbonylamino) phenyl]-6-oxo-5-(5-quinolyl)pyridazin-1-yl]butylamino]-1-methyl-2-oxo-ethyl]carbamoyl]-2-methyl-propyl]ammonium trifluoroacetate (25.4 mg, 31.1 μmol), 1-{6-[(2,5-dioxopyrrolidin-1-yl)oxy]-6-oxohexyl}-1H-pyrrole-2,5-dione (13.4 mg, 43.3 μmol), and N,N-diisopropylethylamine (13 μl, 72 μmol) in DMF (0.56 mL) was stirred at r.t. for 4 h. Then the reaction mixture was diluted with formic acid (2.7 μl, 70 μmol) in water (0.2 mL) and DMSO and was purified by preparative HPLC to give 7.13 mg (80% purity, 17% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-8 min 10% B, 8-11 min 10-20% B 11-12.5 min 20% B, 12.5-19 min 20-40% B; rate 150 mL/min, temperature 25° C.

LC-MS (Method 1): Rₜ=0.82 min; MS (ESIpos): m/z=896 [M+H]⁺

¹H-NMR (400 MHz, DMSO-d6) δ [ppm]: 0.769 (1.19), 0.786 (1.30), 0.798 (1.26), 0.814 (1.22), 1.154 (0.44), 1.170 (1.44), 1.188 (1.30), 1.233 (0.44), 1.418 (0.22), 1.435 (0.48), 1.454 (0.59), 1.471 (0.52), 1.484 (0.41), 1.503 (0.37), 1.846 (0.30), 1.919 (0.19), 1.937 (0.19), 2.078 (0.19), 2.084 (0.56), 2.096 (0.26), 2.112 (0.22), 2.129 (0.26), 2.318 (0.63), 2.660 (0.63), 3.104 (0.19), 3.141 (0.19), 3.157 (0.19), 3.306 (0.41), 3.349 (1.56), 3.366 (0.52), 4.093 (0.22), 4.114 (0.30), 4.131 (0.26), 4.190 (0.26), 4.208 (0.52), 4.226 (0.52), 4.820 (0.67), 4.839 (0.67), 6.984 (3.74), 7.437 (0.37), 7.448 (0.37), 7.505 (0.37), 7.515 (0.37), 7.526 (0.37), 7.537 (0.33), 7.672 (0.41), 7.687 (0.44), 7.705 (0.74), 7.727 (0.85), 7.761 (0.33), 7.782 (0.33), 7.837 (0.56), 7.855 (0.41), 7.858 (0.48), 7.875 (0.33), 7.904 (0.89), 7.915 (0.48), 7.926 (0.81), 7.934 (0.44), 8.076 (0.33), 8.098 (0.30), 8.119 (0.48), 8.140 (0.41), 8.160 (1.15), 8.499 (0.52), 8.512 (0.48), 8.617 (0.70), 8.641 (0.59), 8.936 (0.41), 8.940 (0.44), 8.946 (0.41), 8.951 (0.37).

### Example 27C

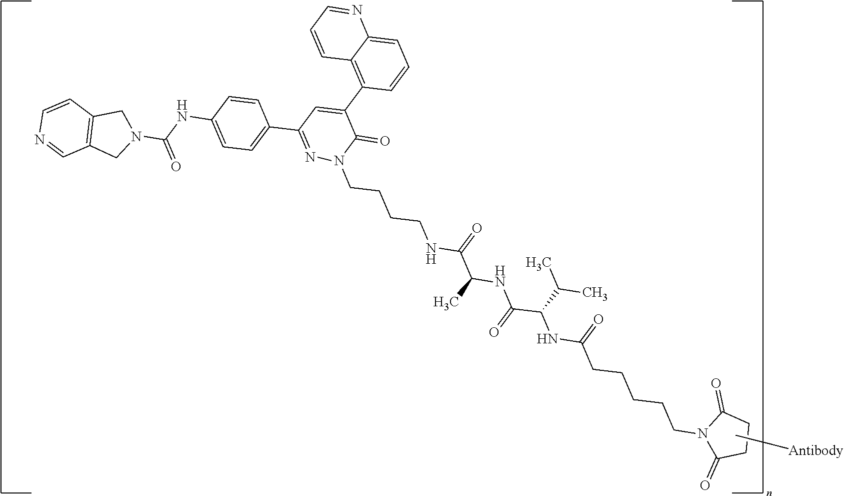

5 mg of anti-B7H3 TPP-8382 (15.1 mg/mL) were coupled with Final Intermediate 27-5 N-[6-(2,5-dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide (300 μg, 80% purity, 0.27 μmol) according to procedure 1 in and the reaction was, after Sephadex purification, concentrated by ultracentrifugation and rediluted with PBS.

Protein concentration: 2.08 mg/mL

Drug/mAb ratio: 1.8 (UV)

### Intermediate 28-1

### Tert-Butyl [(2S)-1-({4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}amino)-1-oxopropan-2-yl]carbamate

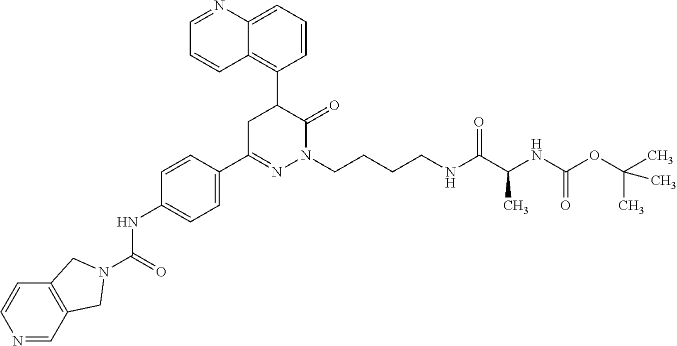 [SMILES: C[C@H](NC(=O)OC(C)(C)C)C(=O)NCCCCN1N=C(c2ccc(NC(=O)N3Cc4ccncc4C3)cc2)CC(c2cccc3ncccc23)C1=O]

To a mixture of N-(tert-butoxycarbonyl)-L-alanine (54.2 mg, 287 μmol), 4-methylmorpholin (56 μl, 510 μmol) and HATU (96.2 mg, 253 μmol) in DMF (1.3 mL, 17 mmol), which had been stirred for 15 min, was added N-{4-[1-(4-aminobutyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 2, 100 mg, 187 μmol) and stirring of the combined mixture was continued at r.t. for 4 h. Then the reaction mixture was diluted with formic acid (19 μl, 510 μmol) in water (0.1 mL) and DMSO and purified by preparative HPLC to give 73.4 mg (95% purity, 53% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-6 min 5-7% B, 6-12 min 7-25% B, 12-14 min 25% B; rate 150 mL/min, temperature 25° C.

LC-MS (Method 2): Rₜ=0.98 min; MS (ESIpos): m/z=705 [M+H]⁺, (ESIneg): m/z=703 [M−H]⁻

¹H-NMR (400 MHz, DMSO-d6) δ [ppm]: 1.132 (3.52), 1.150 (3.46), 1.357 (16.00), 1.453 (0.67), 1.471 (1.01), 1.490 (0.86), 1.681 (0.80), 1.700 (0.97), 1.718 (0.68), 2.070 (0.89), 2.518 (1.55), 2.523 (0.95), 3.072 (0.53), 3.087 (0.49), 3.124 (0.56), 3.139 (0.61), 3.275 (0.70), 3.303 (1.02), 3.317 (1.61), 3.421 (2.38), 3.445 (1.13), 3.464 (0.80), 3.815 (0.52), 3.832 (0.91), 3.844 (0.84), 3.851 (0.82), 3.869 (0.52), 3.887 (0.61), 3.906 (0.72), 4.742 (0.68), 4.760 (0.87), 4.770 (0.83), 4.796 (2.77), 4.814 (2.72), 6.816 (0.79), 6.835 (0.76), 7.421 (1.48), 7.434 (1.77), 7.457 (1.48), 7.549 (1.26), 7.560 (1.27), 7.571 (1.26), 7.581 (1.26), 7.622 (2.62), 7.644 (3.93), 7.701 (1.60), 7.710 (4.09), 7.719 (1.77), 7.722 (2.04), 7.732 (2.46), 7.740 (1.48), 7.787 (0.89), 7.800 (0.53), 7.951 (1.83), 7.972 (1.50), 8.488 (1.72), 8.500 (1.62), 8.585 (1.26), 8.601 (3.13), 8.629 (2.57), 8.910 (1.54), 8.914 (1.62), 8.920 (1.55). 8.924 (1.44).

### Intermediate 28-2

### [(1S)-2-[4-[3-[4-(1,3-Dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]ammonium Trifluoroacetate

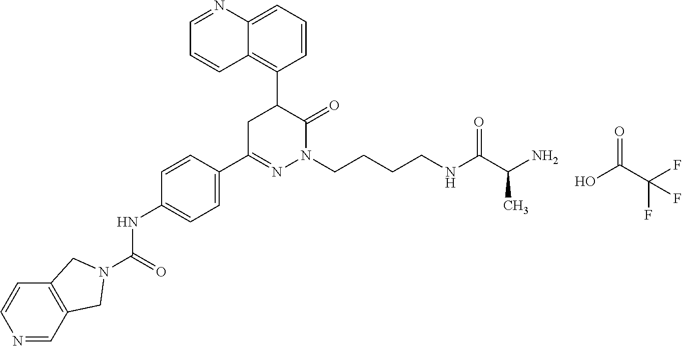 [SMILES: C[C@H](N)C(=O)NCCCCN1N=C(c2ccc(NC(=O)N3Cc4ccncc4C3)cc2)CC(c2cccc3ncccc23)C1=O ; O=C(O)C(F)(F)F]

A mixture of tert-butyl [(2S)-1-({4-[3-{4-[(1,3-dihydro-2H-pyrrolo[3,4-c]pyridine-2-carbonyl) amino]phenyl}-6-oxo-5-(quinolin-5-yl)-5,6-dihydropyridazin-1(4H)-yl]butyl}amino)-1-oxopropan-2-yl]carbamate (73.4 mg, 104 μmol) and trifluoroacetic acid (220 μL, 2.8 mmol) in dichloromethane (0.88 mL) was stirred at r.t. for 2 h. Then the mixture was diluted with toluene, concentrated under reduced pressure (azeotropic distillation with toluene was executed two times) to give 72.0 mg (95% purity, 91% yield) of the title compound.

LC-MS (Method 1): Rₜ=0.56 min, MS (ESIneg): m/z=717 [M−H]⁻

¹H-NMR (400 MHz, DMSO-d6) δ [ppm]: 0.830 (0.98), 0.845 (1.48), 0.863 (2.07), 0.882 (1.77), 0.901 (2.71), 0.919 (1.33), 1.227 (3.40), 1.301 (9.16), 1.319 (9.06), 1.361 (1.03), 1.380 (0.89), 1.388 (0.84), 1.407 (0.84), 1.422 (0.69), 1.511 (2.07), 1.527 (2.71), 1.706 (1.67), 1.725 (1.97), 2.293 (16.00), 3.101 (1.08), 3.136 (0.98), 3.152 (1.33), 3.167 (1.23), 3.192 (1.28), 3.208 (1.38), 3.225 (0.98), 3.301 (0.79), 3.330 (0.94), 3.342 (1.33), 3.372 (1.33), 3.433 (1.33), 3.450 (1.67), 3.474 (1.13), 3.492 (1.03), 3.737 (2.12), 3.749 (2.56), 3.761 (2.81), 3.773 (1.82), 3.806 (1.67), 3.822 (2.07), 3.839 (2.51), 3.853 (2.46), 3.870 (2.81), 3.885 (2.51), 3.902 (2.26), 4.130 (3.89), 4.223 (3.89), 4.229 (3.89), 4.237 (3.79), 4.243 (3.69), 4.786 (1.43), 4.804 (1.62), 4.814 (1.53), 4.833 (1.43), 4.879 (5.22), 4.896 (5.07), 7.138 (1.72), 7.158 (3.50), 7.176 (4.14), 7.226 (3.40), 7.245 (3.74), 7.263 (1.43), 7.509 (2.22), 7.527 (2.41), 7.629 (4.43), 7.651 (7.29), 7.664 (2.02), 7.674 (1.72), 7.717 (1.87), 7.731 (7.83), 7.754 (4.09), 7.775 (1.67), 7.795 (2.41), 7.814 (1.62), 8.002 (4.28), 8.023 (5.71), 8.083 (4.14), 8.340 (0.98), 8.354 (2.02), 8.368 (1.03), 8.653 (2.07), 8.667 (2.07), 8.710 (4.48), 8.741 (1.72), 8.775 (3.40), 8.993 (2.31), 9.001 (2.26).

### Intermediate 28-3

### N-(tert-Butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide

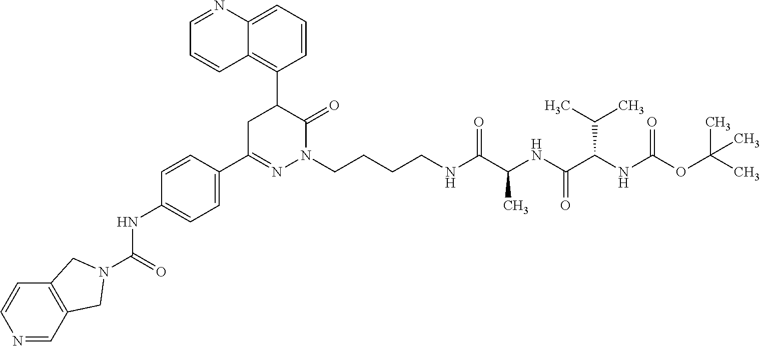 [SMILES: CC(C)[C@H](NC(=O)OC(C)(C)C)C(=O)N[C@@H](C)C(=O)NCCCCN1N=C(c2ccc(NC(=O)N3Cc4ccncc4C3)cc2)CC(c2cccc3ncccc23)C1=O]

To a mixture of N-(tert-butoxycarbonyl)-L-valine (34.6 mg, 159 μmol), 4-methylmorpholin (29 μl, 260 μmol), and HATU (53.5 mg, 141 μmol) in DMF (0.70 mL), which had been stirred for min, was added [(1S)-2-[4-[3-[4-(1,3-dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]ammonium trifluoroacetate (74.9 mg, 104 μmol) in DMF (0.7 mL) containing 4-methylmorpholin (16 μl, 155 μmol) and stirring of the combined mixture was continued at r.t. for 3 h. Then 4-methylmorpholin (46 μl, 420 μmol) was added and the addition of N-(tert-butoxycarbonyl)-L-valine (34.6 mg, 159 μmol), 4-methylmorpholin (46 μl, 420 μmol) and HATU (53.5 mg, 141 μmol) in DMF (0.50 mL) was repeated after 2 h. The reaction mixture was diluted with formic acid (59 μl, 1.6 mmol) in water (0.1 mL) and DMSO (3 mL) and purified by preparative HPLC to give 78.4 mg (90% purity, 84% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm; Eluent A: water+0.1% formic acid; Eluent B: acetonitrile; gradient: 0-8 min 7% B, 8-20 min 7-25% B, 20-25 min 25% B; rate 150 mL/min, temperature 25° C.

LC-MS (Method 1): Rₜ=0.88 min, MS (ESipos): m/z=804 [M+H]⁺, (ESIneg): m/z=802 [M−H]⁻

¹H-NMR (400 MHz, DMSO-d6) δ [ppm]: 0.773 (2.34), 0.790 (2.60), 0.816 (3.17), 0.832 (3.12), 1.163 (3.95), 1.181 (4.05), 1.366 (16.00), 1.448 (0.57), 1.468 (0.83), 1.485 (0.88), 1.676 (0.73), 1.695 (0.83), 1.713 (0.57), 2.068 (8.26), 2.725 (0.68), 2.727 (0.68), 2.886 (0.83), 3.136 (0.52), 3.304 (0.62), 3.319 (0.88), 3.348 (1.77), 3.443 (1.82), 3.461 (0.99), 3.501 (0.62), 3.777 (0.62), 3.794 (0.52), 3.816 (0.62), 3.834 (0.83), 3.852 (0.68), 4.249 (0.62), 4.740 (0.57), 4.758 (0.73), 4.767 (0.68), 4.796 (2.23), 4.814 (2.23), 6.758 (0.68), 6.780 (0.68), 7.421 (1.25), 7.435 (1.82), 7.455 (1.30), 7.547 (1.14), 7.558 (1.19), 7.570 (1.19), 7.580 (1.14), 7.620 (2.23), 7.642 (3.43), 7.701 (1.56), 7.706 (3.64), 7.722 (1.77), 7.728 (2.18), 7.739 (0.99), 7.849 (0.73), 7.868 (0.68), 7.912 (0.68), 7.950 (1.66), 7.971 (1.35), 8.486 (1.25), 8.499 (1.25), 8.584 (1.04), 8.600 (2.18), 8.642 (2.18), 8.908 (1.40), 8.912 (1.45), 8.919 (1.40), 8.922 (1.30).

### Intermediate 28-4

### [(1S)-1-[[(1S)-2-[4-[3-[4-(1,3-Dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]carbamoyl]-2-methyl-propyl]ammonium Trifluoroacetate

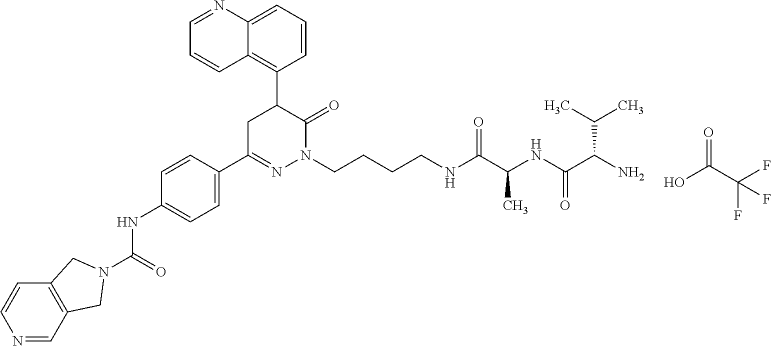 [SMILES: CC(C)[C@H](N)C(=O)N[C@@H](C)C(=O)NCCCCN1N=C(c2ccc(NC(=O)N3Cc4ccncc4C3)cc2)CC(c2cccc3ncccc23)C1=O ; O=C(O)C(F)(F)F]

A mixture of N-(tert-butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide (24.2 mg, 30.1 μmol) and trifluoroacetic acid (63 μl, 810 μmol) in dichloromethane (0.43 mL) was stirred at r.t. for 4.5 h. Then the mixture was diluted with toluene and concentrated under reduced pressure (azeotropic distillation with toluene was conducted twice) to give 22.8 mg (90% purity, 83% yield) of the title compound.

LC-MS (Method 1): Rₜ=0.60 min: MS (ESIneg): m/z=816 [M−H]⁻

¹H-NMR (400 MHz, DMSO-d6) δ [ppm]: 0.782 (9.71), 0.799 (10.00), 0.864 (9.29), 0.881 (9.43), 1.177 (10.71), 1.195 (10.71), 1.459 (1.43), 1.477 (2.00), 1.496 (1.71), 1.515 (0.71), 1.684 (1.71), 1.703 (2.14), 1.722 (1.43), 1.924 (0.86), 1.941 (1.14), 1.955 (1.14), 1.972 (0.71), 2.073 (6.14), 3.079 (1.00), 3.096 (1.57), 3.110 (1.71), 3.125 (1.86), 3.141 (2.57), 3.157 (2.57), 3.409 (3.29), 3.426 (2.71), 3.450 (1.57), 3.469 (1.29), 3.505 (0.71), 3.811 (1.00), 3.829 (1.86), 3.849 (1.71), 3.870 (0.86), 4.273 (1.29), 4.290 (1.86), 4.308 (1.29), 4.747 (1.43), 4.765 (1.86), 4.775 (1.86), 4.796 (5.71), 4.816 (5.43), 7.424 (3.00), 7.437 (3.57), 7.460 (3.14), 7.549 (2.86), 7.559 (2.86), 7.571 (2.86), 7.581 (2.86), 7.625 (5.57), 7.648 (8.29), 7.703 (2.86), 7.713 (8.71), 7.724 (3.86), 7.736 (5.14), 7.742 (2.86), 7.953 (3.86), 7.974 (4.14), 7.987 (2.43), 8.000 (1.14), 8.172 (2.86), 8.195 (1.14), 8.491 (4.57), 8.504 (4.29), 8.587 (2.43), 8.605 (7.43), 8.628 (5.29), 8.912 (3.57), 8.916 (3.57), 8.922 (3.43), 8.926 (3.00).

### Final Intermediate 28-5

N-[6-(2,5-dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide

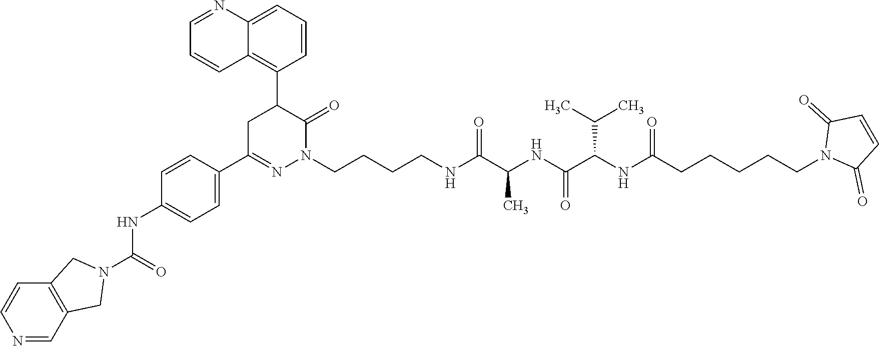 [SMILES: CC(C)[C@H](NC(=O)CCCCCN1C(=O)C=CC1=O)C(=O)N[C@@H](C)C(=O)NCCCCN1N=C(c2ccc(NC(=O)N3Cc4ccncc4C3)cc2)CC(c2cccc3ncccc23)C1=O]

A mixture of [(1S)-1-[[(1S)-2-[4-[3-[4-(1,3-dihydropyrrolo[3,4-c]pyridine-2-carbonylamino) phenyl]-6-oxo-5-(5-quinolyl)-4,5-dihydropyridazin-1-yl]butylamino]-1-methyl-2-oxo-ethyl]carbamoyl]-2-methyl-propyl]ammonium trifluoroacetate (24.7 mg, 30.3 μmol), 1-{6-[(2,5-dioxopyrrolidin-1-yl)oxy]-6-oxohexyl}-1H-pyrrole-2,5-dione (13.0 mg, 42.1 μmol), and N,N-diisopropylethylamine (12 μl, 70 μmol) in DMF (0.54 mL) was stirred at r.t. for 4 h. Then the reaction mixture was diluted with formic acid (2.6 μl. 70 μmol) in water (0.2 mL) and DMSO and purified by preparative HPLC to give 2.54 mg (90% purity, 6% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm; Eluent A: water+0.1% formic acid; Eluent B: acetonitrile; gradient: 0-8 min 10% B, 8-11 min 10-20% B 11-12.5 min 20% B, 12.5-19 min 20-40% B; rate 150 mL/min, temperature 25° C.

LC-MS (Method 1): Rₜ=0.81 min; MS (ESIpos): m/z=449.6 [M+2H]²⁺, (ESIneg): m/z=896 [M−H]⁻

¹H-NMR (400 MHz, DMSO-d6) δ [ppm]: 0.779 (3.02), 0.796 (3.32), 0.807 (3.62), 0.825 (3.32), 1.139 (0.60), 1.166 (3.92), 1.184 (3.92), 1.229 (0.60), 1.441 (1.81), 1.460 (2.42), 1.470 (2.11), 1.675 (0.60), 1.695 (0.91), 1.930 (0.60), 1.946 (0.60), 2.070 (8.75), 2.082 (0.91), 2.102 (0.60), 2.119 (0.60), 2.138 (0.91), 2.156 (0.60), 3.051 (0.60), 3.067 (0.60), 3.082 (0.60), 3.098 (0.30), 3.119 (0.60), 3.134 (0.60), 3.152 (0.60), 3.168 (0.60), 3.231 (0.60), 3.326 (3.02), 3.334 (4.83), 3.835 (0.91), 4.092 (0.60), 4.113 (0.91), 4.130 (0.60), 4.189 (0.60), 4.207 (0.91), 4.225 (0.60), 4.741 (0.60), 4.759 (0.60), 4.768 (0.60), 4.797 (2.11), 4.815 (2.11), 6.980 (10.87), 7.422 (1.21), 7.437 (1.51), 7.459 (1.21), 7.548 (0.91), 7.558 (0.91), 7.569 (0.91), 7.580 (0.91), 7.619 (1.81), 7.642 (2.72), 7.700 (1.51), 7.707 (3.02), 7.721 (1.81), 7.730 (2.11), 7.740 (1.21), 7.783 (0.91), 7.805 (1.51), 7.817 (1.21), 7.830 (0.60), 7.909 (0.91), 7.928 (0.91), 7.950 (1.51), 7.971 (1.21), 8.488 (1.51), 8.500 (1.51), 8.585 (0.91), 8.601 (2.72), 8.628 (1.81), 8.909 (1.21), 8.912 (1.21), 8.919 (1.21), 8.923 (1.21).

### Example 28D

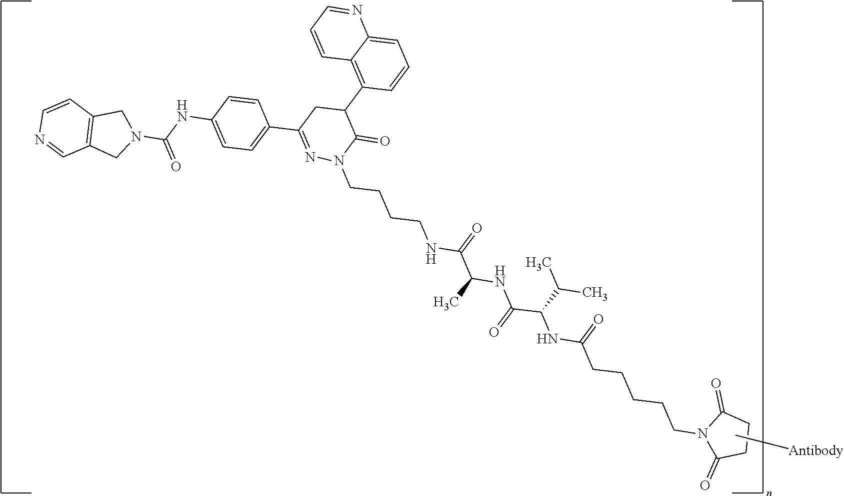

5 mg of anti-C4.4a TPP-509 (9.87 mg/mL) were coupled with Final Intermediate 28-5 N-[6-(2,5-dioxo-2,5-dihydro-1H-pyrrol-1-yl)hexanoyl]-L-valyl-N-{4-[3-{4-[(1,3-dihydro-2H-pyrrolo [3,4-c]pyridine-2-carbonyl)amino]phenyl}-6-oxo-5-(quinolin-5-yl)-5,6-dihydropyridazin-1(4H)-yl]butyl}-L-alaninamide (270 μg, 90% purity, 0.27 μmol) according to procedure 1 in potassium phosphate buffer (0.1 M) and the reaction was, after Sephadex purification, concentrated by ultracentrifugation and rediluted with potassium phosphate buffer (0.1 M).

Protein concentration: 1.01 mg/mL

Drug/mAb ratio: 2.7 (LCMS)

### Final Intermediate 29-1

### N-{4-[1-(6-{[6-(2,5-Dioxo-2,5-dihydro-1H-pyrrol-1-yl)hexanoyl]amino}hexyl)-6-oxo-5-(quinolin-5-yl)-1,6-dihydropyridazin-3-yl]phenyl}-1,3-dihydro-2H-pyrrolo[3,4-c]pyridine-2-carboxamide

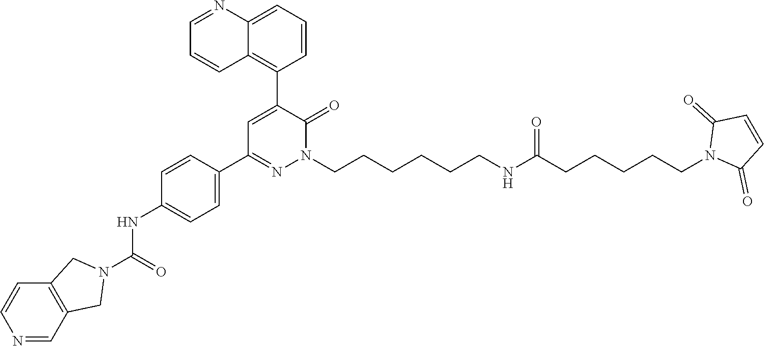 [SMILES: O=C(CCCCCN1C(=O)C=CC1=O)NCCCCCCn1nc(-c2ccc(NC(=O)N3Cc4ccncc4C3)cc2)cc(-c2cccc3ncccc23)c1=O]

A mixture of N-{4-[1-(6-aminohexyl)-6-oxo-5-(quinolin-5-yl)-1,6-dihydropyridazin-3-yl]phenyl}-1,3-dihydro-2H-pyrrolo[3,4-c]pyridine-2-carboxamide (see Example 6, 23.0 mg, 41.1 μmol), 1-{6-[(2,5-dioxopyrrolidin-1-yl)oxy]-6-oxohexyl}-1H-pyrrole-2,5-dione (15.2 mg, 49.3 μmol) and N,N-diisopropylethylamine (14 μl, 82 μmol) in DMF (630 μl) was stirred at r.t. for 4 h.

Then water (0.2 mL) and formic acid (3.1 μl, 82 μmol) were added to the reaction solution, the mixture was filtered and the filtrate purified by preparative HPLC to give 8 mg of the title compound (80% purity, 20% yield)

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-6 min 5-7% B, 6-12 min 7-30% B, 12-15 min 30% B; rate 150 mL/min, temperature 25° C.

LC-MS (Method 1): Rt=0.84 min; MS (ESIneg): m/z=751 [M−H]⁻

¹H-NMR (400 MHz, DMSO-d6) δ [ppm]: 1.106 (0.99), 1.124 (1.38), 1.144 (2.57), 1.161 (1.98), 1.181 (0.99), 1.231 (0.99), 1.358 (4.74), 1.377 (4.54), 1.407 (3.16), 1.432 (3.75), 1.450 (4.35), 1.463 (3.16), 1.833 (1.78), 1.850 (2.17), 1.973 (2.57), 1.991 (4.35), 2.009 (2.17), 2.072 (13.63), 2.326 (2.77), 2.331 (2.17), 2.665 (1.98), 2.669 (2.77), 2.673 (2.17), 3.003 (2.77), 3.017 (2.77), 4.228 (2.17), 4.819 (4.94), 4.837 (4.74), 6.975 (16.00), 7.436 (2.37), 7.448 (2.37), 7.499 (1.98), 7.509 (1.98), 7.521 (1.98), 7.531 (1.98), 7.676 (2.77), 7.693 (4.15), 7.700 (5.53), 7.722 (6.91), 7.835 (1.98), 7.856 (2.77), 7.874 (2.17), 7.898 (5.33), 7.920 (4.35), 8.074 (2.37), 8.095 (2.37), 8.116 (3.16), 8.137 (2.57), 8.152 (6.52), 8.498 (2.96), 8.510 (2.77), 8.616 (4.54), 8.643 (3.95), 8.932 (2.37), 8.936 (2.57), 8.943 (2.37), 8.946 (2.37).

### Example 29C

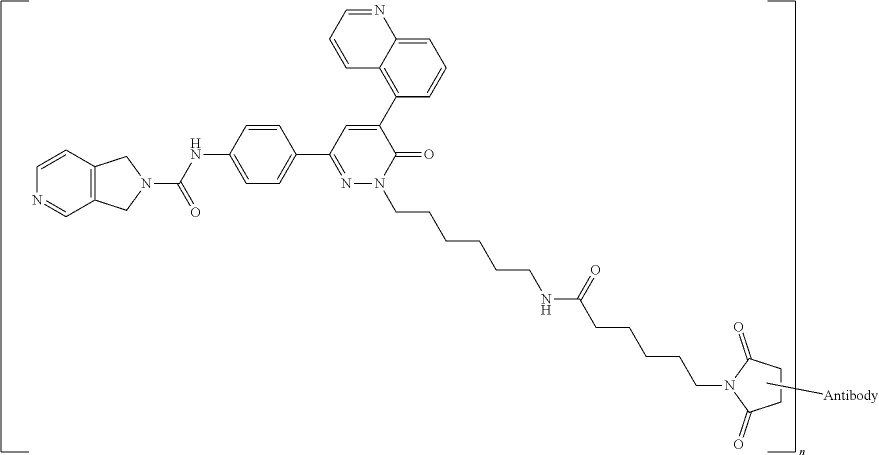

5 mg of anti-B7H3 TPP-8382 (15.1 mg/mL) were coupled with Final Intermediate 29-1 N-{4-[1-(6-{[6-(2,5-dioxo-2,5-dihydro-1H-pyrrol-1-yl)hexanoyl]amino}hexyl)-6-oxo-5-(quinolin-5-yl)-1,6-dihydropyridazin-3-yl]phenyl}-1,3-dihydro-2H-pyrrolo[3,4-c]pyridine-2-carboxamide (250 μg, 80% purity, 0.27 μmol) according to procedure 1 and the reaction was, after Sephadex purification, concentrated by ultracentrifugation and rediluted with PBS.

Protein concentration: 1.70 mg/mL

Drug/mAb ratio: 0.7 (UV)

### Example 29D

5 mg of anti-C4.4a TPP-509 (9.87 mg/mL) were coupled with Final Intermediate 29-1 N-{4-[1-(6-{[6-(2,5-dioxo-2,5-dihydro-1H-pyrrol-1-yl)hexanoyl]amino}hexyl)-6-oxo-5-(quinolin-5-yl)-1,6-dihydropyridazin-3-yl]phenyl}-1,3-dihydro-2H-pyrrolo[3,4-c]pyridine-2-carboxamide (250 μg, 80% purity, 0.27 μmol) according procedure 1 and the reaction was, after Sephadex purification, concentrated by ultracentrifugation and rediluted with PBS.

Protein concentration: 0.98 mg/mL

Drug/mAb ratio: 3.9 (UV)

### Example 29M

### S-{1-[6-({6-[3-{4-[(1,3-Dihydro-2H-pyrrolo[3,4-c]pyridine-2-carbonyl)amino]phenyl}-6-oxo-5-(quinolin-5-yl)pyridazin-1(6H)-yl]hexyl}amino)-6-oxohexyl]-2,5-dioxopyrrolidin-3-yl}-L-cysteine

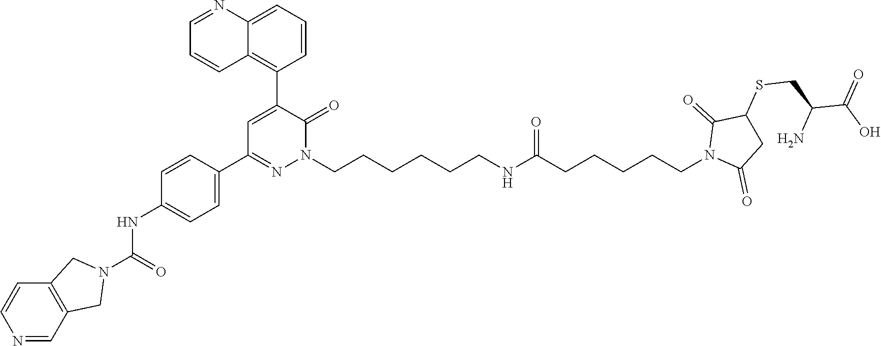 [SMILES: N[C@@H](CSC1CC(=O)N(CCCCCC(=O)NCCCCCCn2nc(-c3ccc(NC(=O)N4Cc5ccncc5C4)cc3)cc(-c3cccc4ncccc34)c2=O)C1=O)C(=O)O]

To a solution of N-{4-[1-(6-{[6-(2,5-dioxo-2,5-dihydro-1H-pyrrol-1-yl)hexanoyl]amino}hexyl)-6-oxo-5-(quinolin-5-yl)-1,6-dihydropyridazin-3-yl]phenyl}-1,3-dihydro-2H-pyrrolo[3,4-c]pyridine-2-carboxamide (see Final Intermediate 29-1, 3.00 mg, 80% purity, 3.19 μmol) in DMF (260 μl) and water (26 μl) was added at r.t. L-cysteine (390 μg, 3.2 μmol) and the mixture stirred for 4 h at that temperature. After that the mixture was purified by preparative HPLC to give 2.01 mg of the title compound (95% purity, 69% yield).

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm; Eluent A: water+0.1% formic acid; Eluent B: acetonitrile; gradient: 0-14 min 5-30% B, 14-17 min 30% B, rate 60 mL/min, temperature 25° C.

LC-MS (Method 1): Rt=0.72 min; MS (ESIneg): m/z=872 [M−H]⁻

¹H-NMR (400 MHz, DMSO-d6) δ [ppm]: 1.151 (1.38), 1.171 (1.34), 1.363 (3.46), 1.390 (2.81), 1.406 (2.81), 1.422 (3.01), 1.436 (3.13), 1.454 (2.73), 1.472 (1.51), 1.833 (1.30), 1.852 (1.67), 1.983 (1.79), 2.000 (3.05), 2.019 (1.51), 2.074 (16.00), 2.084 (0.94), 2.518 (10.18), 2.523 (7.57), 2.534 (1.59), 2.540 (1.71), 2.572 (0.81), 2.582 (0.81), 2.678 (0.77), 2.808 (0.65), 2.995 (1.02), 3.011 (2.28), 3.025 (2.28), 3.038 (1.18), 3.072 (0.77), 3.091 (0.81), 3.110 (1.26), 3.132 (2.04), 3.142 (1.06), 3.156 (1.38), 3.167 (0.77), 3.178 (1.63), 4.012 (0.77), 4.022 (0.81), 4.035 (0.81), 4.044 (0.69), 4.077 (0.65), 4.090 (0.69), 4.226 (1.83), 4.830 (3.83), 4.848 (3.75), 7.434 (2.32), 7.447 (2.32), 7.505 (2.12), 7.516 (2.08), 7.526 (2.16), 7.536 (2.04), 7.676 (2.28), 7.679 (2.48), 7.694 (2.85), 7.697 (2.85), 7.717 (4.68), 7.740 (5.21), 7.809 (0.90), 7.838 (2.40), 7.856 (2.16), 7.859 (2.69), 7.877 (2.08), 7.897 (5.58), 7.919 (4.76), 8.077 (2.04), 8.096 (1.91), 8.118 (2.73), 8.139 (2.44), 8.154 (6.60), 8.498 (3.30), 8.510 (2.97), 8.615 (4.48), 8.751 (2.08), 8.759 (1.75), 8.936 (2.48), 8.940 (2.61), 8.946 (2.36), 8.950 (2.16).

### Final Intermediate 30-1

### N-{4-[1-{6-[2-(2,5-Dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide

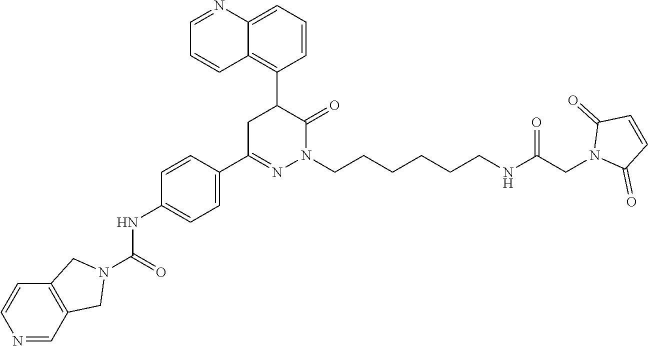 [SMILES: O=C(CN1C(=O)C=CC1=O)NCCCCCCN1N=C(c2ccc(NC(=O)N3Cc4ccncc4C3)cc2)CC(c2cccc3ncccc23)C1=O]

A mixture of N-{4-[1-(6-aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 3, 23.5 mg, 95% purity, 39.7 μmol), 1-{2-[(2,5-dioxopyrrolidin-1-yl)oxy]-2-oxoethyl}-1H-pyrrole-2,5-dione (10.0 mg, 39.7 μmol), and N,N-diisopropylethylamine (14 μl, 79 μmol) in DMF (760 μl) was stirred at r.t. for 10 min. Then toluene (50 mL) and formic acid (3.0 μl, 79 μmol) were added to the reaction solution and the mixture was concentrated under vacuum. The crude product was purified by Isolera (4 g 15 μm silicagel, eluent: dichloromethane/isopropyl alcohol, gradient) to give 22 mg of the desired product (95% purity, 74% yield).

LC-MS (Method 1): Rt=0.71 min, MS (ESIneg): m/z=697 [M−H]⁻

¹H-NMR (400 MHz, DMSO-d6) δ [ppm]: 1.327 (1.76), 1.381 (0.69), 1.397 (0.81), 1.413 (0.54), 1.686 (0.57), 1.703 (0.75), 2.084 (0.57), 2.331 (0.96), 2.518 (6.39), 2.522 (3.85), 2.673 (0.96), 3.028 (1.10), 3.043 (1.10), 3.291 (0.51), 3.360 (0.90), 3.406 (0.57), 3.423 (0.66), 3.779 (0.63), 3.789 (0.63), 3.825 (0.87), 3.842 (0.87), 3.987 (5.19), 4.752 (0.48), 4.770 (0.63), 4.779 (0.66), 4.798 (2.24), 4.818 (1.91), 7.085 (9.19), 7.424 (1.04), 7.436 (1.16), 7.443 (1.07), 7.462 (1.07), 7.542 (0.87), 7.552 (0.90), 7.564 (0.90), 7.574 (0.90), 7.627 (1.76), 7.650 (2.72), 7.699 (0.93), 7.714 (2.93), 7.736 (1.94), 7.950 (1.25), 7.971 (1.01), 8.098 (0.81), 8.491 (1.40), 8.503 (1.31), 8.592 (0.93), 8.604 (2.18), 8.626 (1.85), 8.911 (1.04), 8.914 (1.07), 8.921 (1.10), 8.924 (0.96).

### Example 30Aa

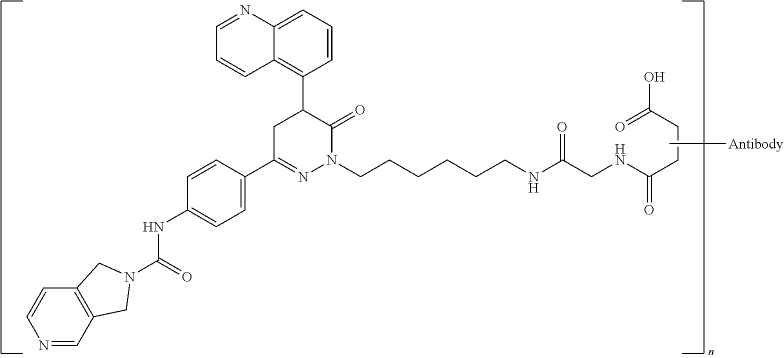

5 mg of anti-HER2 TPP-1015 (c=12.2 mg/mL) were coupled with Final Intermediate 30-1 N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (200 μg, 95% purity, 0.27 μmol) according to procedure 2 and the reaction was, after Sephadex purification, concentrated by ultracentrifugation and rediluted with PBS.

Protein concentration: 1.34 mg/mL

Drug/mAb ratio: 4.3 (UV)

### Example 30Ab

5 mg of anti-HER2 TPP-1015 (c=12.2 mg/mL) were coupled with Final Intermediate 30-1 N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (590 μg, 95% purity, 0.80 μmol) according to procedure 2 and the reaction was, after Sephadex purification, concentrated by ultracentrifugation and rediluted with PBS.

Protein concentration: 0.66 mg/mL

Drug/mAb ratio: 9.4 (UV)

### Example 30Ca

5 mg of anti-B7H3 TPP-8382 (c=15.1 mg/mL) were coupled with Final Intermediate 30-1 N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (210 μg, 90% purity, 0.27 μmol) according procedure 2 and the reaction was, after Sephadex purification, concentrated by ultracentrifugation and rediluted with PBS.

Protein concentration: 1.47 mg/mL

Drug/mAb ratio: 4.5 (UV)

### Example 30Cb

5 mg of anti-B7H3 TPP-8382 (c=15.1 mg/mL) were coupled with Final Intermediate 30-1 N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (390 μg, 95% purity, 0.53 μmol) according procedure 2 and the reaction was, after Sephadex purification, concentrated by ultracentrifugation and rediluted with PBS.

Protein concentration: 0.75 mg/mL

Drug/mAb ratio: 8.4 (UV)

### Example 30D

5 mg of anti-C4.4a TPP-509 (c=9.87 mg/mL) were coupled with Final Intermediate 30-1 N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (210 μg, 90% purity, 0.27 μmol) according procedure 2 and the reaction was, after Sephadex purification, concentrated by ultracentrifugation and rediluted with PBS.

Protein concentration: 0.84 mg/mL

Drug/mAb ratio: 3.0 (UV)

### Example 30E

5 mg of anti-C4.4a TPP-668 (c=11.62 mg/mL) were coupled with Final Intermediate 30-1 N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (210 μg, 90% purity, 0.27 μmol) according procedure 2 and the reaction was, after Sephadex purification, concentrated by ultracentrifugation and rediluted with PBS.

Protein concentration: 1.80 mg/mL

Drug/mAb ratio: 3.2 (UV)

### Intermediate 30-2

### N-{[2-(Trimethylsilyl)ethoxy]carbonyl}-L-cysteine

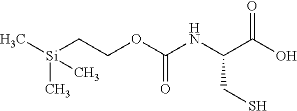 [SMILES: C[Si](C)(C)CCOC(=O)N[C@@H](CS)C(=O)O]

A mixture of L-cysteine (CAS52-90-4, 470 mg, 3.88 mmol), 1-({[2-(trimethylsilyl)-ethoxy]carbonyl}oxy)pyrrolidine-2,5-dione (CAS 78269-85-9, 1.16 g, 4.46 mmol) and N,N-diisopropylethylamine (2.0 mL, 12 mmol) in DMF (12 mL) was stirred at r.t. for 3 days. Then toluene (100 mL) was added to the reaction solution and the mixture was concentrated under reduced pressure (azeotropic distillation with toluene was executed two times). The crude product was dissolved in DMSO and purified by preparative HPLC to give 770 mg of the title compound (90% purity, 67% yield)

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 290×51 mm; Eluent A: water+0.1% formic acid; Eluent B: acetonitrile; gradient: 0-20 min 10-70% B, 20-25 min 70% B.; rate 250 mL/min, temperature 25° C.

LC-MS (Method 1): Rt=0.81 min; MS (ESIneg): m/z=264 [M−H]⁻

¹H-NMR (400 MHz, DMSO-d6) δ [ppm]: 0.03 (s, 9H), 0.93 (t, 2H), 2.65-2.75 (m, 1H), 2.81-2.90 (m, 1H), 4.03-4.11 (m, 1H), 4.05 (t, 2H), 7.35 (d, 1H), 12.81 (br s, 1H).

### Intermediate 30-3

### S-{1-[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]-2,5-dioxopyrrolidin-3-yl}-N-{[2-(trimethylsilyl)ethoxy]carbonyl}-L-cysteine

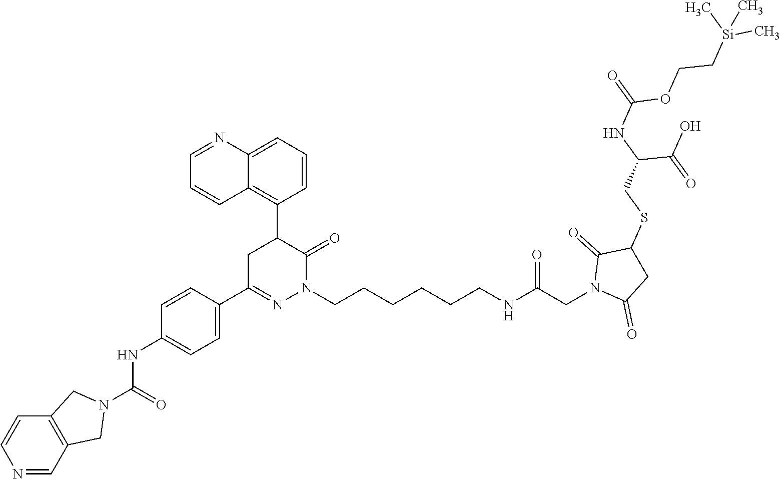 [SMILES: C[Si](C)(C)CCOC(=O)N[C@@H](CSC1CC(=O)N(CC(=O)NCCCCCCN2N=C(c3ccc(NC(=O)N4Cc5ccncc5C4)cc3)CC(c3cccc4ncccc34)C2=O)C1=O)C(=O)O]

To a solution of N-{4-[1-{6-[2-(2,5-dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Final Intermediate 30-1, 13.0 mg, 17.7 μmol) in DMF (2.0 mL) was added N-{[2-(trimethylsilyl)ethoxy]carbonyl}-L-cysteine (see Intermediate 30-2, 9.38 mg, 35.3 μmol) and the mixture was stirred for 4 h at r.t. and 3 days at 6° C. Then to the reaction solution toluene was added and the mixture was concentrated under reduced pressure (azeotropic distillation with toluene was executed two times) to give the crude product (quant.) which was directly used in the next step.

LC-MS (Method 2): Rt=0.83 min; MS (ESIpos): m/z=964 [M+H]⁺ MS (ESIneg): m/z=962 [M−H]⁻

### Intermediate 30-4

### 2 or 3-{[(2R)-2-carboxy-2-({[2-(trimethylsilyl)ethoxy]carbonyl}amino)ethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic Acid

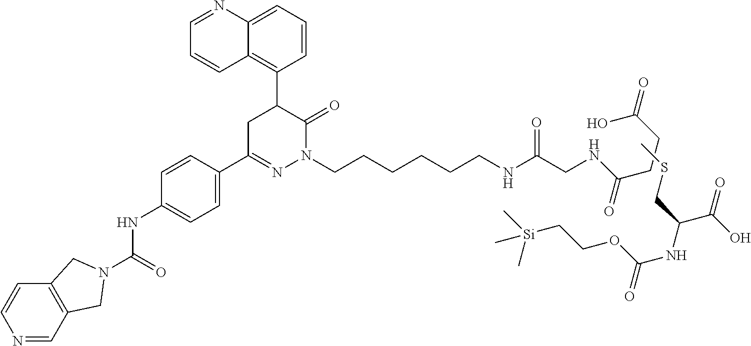

To S-{1-[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]-2,5-dioxopyrrolidin-3-yl}-N-{[2-(trimethylsilyl)ethoxy]carbonyl}-L-cysteine (see crude Intermediate 30-3, 18.0 μmol) in tetrahydrofuran/water was added lithium hydroxide (2.59 mg, 108 μmol) in water (1.2 mL) and the mixture was stirred at r.t. for 1 h. The mixture was neutralized with formic acid (5.4 μl, 140 μmol) and purified by preparative HPLC to give to give 11.5 mg (95% purity, 62% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm. Eluent A: water+0.1% formic acid; Eluent B: acetonitrile; gradient: 0-14 min 15-55% B, 14-17 min 55-100% B, rate 60 mL/min, temperature 25° C.

LC-MS (Method 1): Rt=0.91 min; MS (ESIneg): m/z=980 [M−H]⁻

¹H-NMR (400 MHz, DMSO-d6) δ [ppm]: 0.882 (0.92), 0.903 (0.99), 0.924 (0.85), 1.016 (0.63), 1.031 (0.63), 1.222 (0.92), 1.317 (1.48), 1.355 (0.85), 1.389 (0.63), 1.691 (0.63), 2.325 (1.27), 2.507 (16.00), 2.512 (10.57), 2.528 (1.13), 2.667 (1.41), 3.023 (0.92), 3.037 (0.99), 3.396 (0.70), 3.413 (0.70), 3.615 (0.85), 3.813 (0.70), 3.830 (0.70), 3.993 (1.06), 4.014 (1.13), 4.035 (1.06), 4.760 (0.56), 4.769 (0.49), 4.789 (1.76), 4.808 (1.62), 7.413 (0.99), 7.427 (1.06), 7.450 (0.92), 7.534 (0.85), 7.545 (0.85), 7.556 (0.92), 7.566 (0.85), 7.618 (1.48), 7.641 (2.33), 7.690 (0.99), 7.701 (2.68), 7.708 (1.62), 7.711 (1.55), 7.724 (1.69), 7.729 (1.20), 7.939 (1.06), 7.960 (0.92), 8.480 (1.20), 8.492 (1.13), 8.584 (0.78), 8.594 (1.83), 8.604 (0.85), 8.621 (1.13), 8.900 (0.99), 8.904 (1.06), 8.911 (0.99), 8.914 (0.85).

### Example 30M

### 2 or 3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic Acid

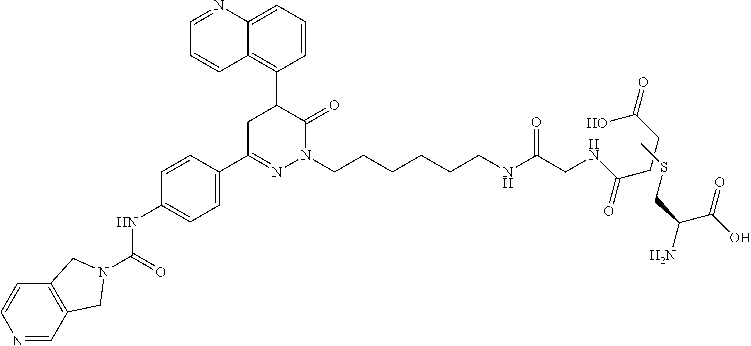

To 2 or 3-{[(2R)-2-carboxy-2-({[2-(trimethylsilyl)ethoxy]carbonyl}amino)ethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic acid (see Intermediate 30-4, 11.5 mg, 11.7 μmol) in 2,2,2-trifluoroethanol (1.6 mL) anhydrous zinc chloride (6.38 mg, 46.8 μmol) was added and stirred at 50° C. for 17 h. Then the mixture was cooled to r.t., 2,2′,2″,2′″-(ethane-1,2-diyldinitrilo)tetraacetic acid (13.7 mg, 46.8 μmol), aqueous formic acid (0.1%, 1 mL) and dimethyl sulfoxide were added and the mixture was purified by preparative HPLC to give to give 3.9 mg (90% purity, 36% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm. Eluent A: water+0.1% formic acid; Eluent B: acetonitrile; gradient: 0-7.5 min 1-16.7% B, 7.5-8.5 min 16.7% B, 8.5-14.5 min 16.7-30% B, 14.5-20 min 30% B, rate 60 mL/min, temperature 25° C.

LC-MS (Method 1): Rt=0.60 min; MS (ESIneg): m/z=836 [M−H]⁻

¹H-NMR (400 MHz, DMSO-d6) δ [ppm]: 1.230 (3.05), 1.326 (8.46), 1.365 (4.09), 1.405 (3.74), 1.688 (2.88), 1.704 (3.74), 2.073 (3.57), 2.083 (0.98), 2.322 (2.42), 2.326 (3.22), 2.332 (2.42), 2.522 (16.00), 2.539 (7.14), 2.565 (1.55), 2.608 (0.75), 2.664 (2.88), 2.669 (4.43), 2.673 (4.03), 2.694 (1.78), 2.710 (1.55), 2.734 (1.38), 2.798 (0.81), 2.820 (0.98), 2.862 (1.38), 2.881 (1.32), 2.897 (1.44), 2.919 (1.50), 2.938 (0.98), 2.957 (1.04), 2.975 (1.32), 3.037 (4.43), 3.050 (4.83), 3.080 (4.60), 3.094 (4.14), 3.200 (5.47), 3.625 (5.64), 3.641 (7.65), 3.656 (4.60), 3.684 (3.68), 3.699 (3.40), 3.708 (3.17), 3.726 (4.09), 3.737 (2.47), 3.760 (3.05), 3.773 (2.71), 3.791 (2.01), 3.807 (2.65), 3.824 (4.43), 3.841 (4.32), 3.857 (2.36), 3.874 (1.38), 3.929 (1.38), 4.752 (2.36), 4.770 (3.11), 4.779 (3.05), 4.805 (9.09), 4.823 (8.81), 7.422 (4.83), 7.435 (5.35), 7.443 (5.12), 7.461 (5.18), 7.547 (4.09), 7.557 (4.09), 7.568 (4.20), 7.578 (3.86), 7.634 (6.16), 7.657 (10.30), 7.702 (5.58), 7.711 (13.53), 7.723 (6.96), 7.734 (7.65), 7.741 (4.78), 7.785 (0.81), 7.798 (1.50), 7.813 (1.55), 7.827 (1.67), 7.841 (0.86), 7.952 (5.70), 7.973 (4.72), 8.243 (0.75), 8.257 (1.50), 8.274 (1.32), 8.289 (1.67), 8.303 (0.86), 8.490 (5.76), 8.503 (5.35), 8.602 (9.44), 8.616 (4.26), 8.678 (3.45), 8.683 (3.68), 8.701 (1.90), 8.912 (5.01), 8.916 (5.24), 8.923 (4.89), 8.926 (4.49).

### Final Intermediate 31-1

### N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide

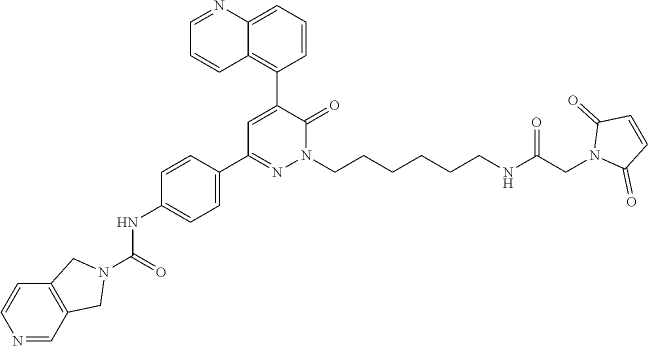 [SMILES: O=C(CN1C(=O)C=CC1=O)NCCCCCCn1nc(-c2ccc(NC(=O)N3Cc4ccncc4C3)cc2)cc(-c2cccc3ncccc23)c1=O]

A mixture of N-{4-[1-(6-aminohexyl)-6-oxo-5-(quinolin-5-yl)-1,6-dihydropyridazin-3-yl]phenyl}-1,3-dihydro-2H-pyrrolo[3,4-c]pyridine-2-carboxamide (see Example 6, 30.0 mg, 95% purity, 50.9 μmol), 6-Maleimidohexanoic acid N-hydroxysuccinimide ester (12.8 mg, 50.9 μmol) and N,N-diisopropylethylamine (18 μl, 100 μmol) in DMF (3.4 mL) was stirred at r.t. for 1 h. Then to the reaction solution toluene (100 mL) and formic acid (3.8 μl, 100 μmol) were added and the mixture was concentrated under vacuum. The crude product was purified by column chromatography (SiO₂, eluent: dichloromethane/isopropyl alcohol) to give 13.2 mg of the desired product (90% purity, 33% yield).

LC-MS (Method 1): Rt=0.75 min: MS (ESIneg): m/z=695 [M−H]⁻

¹H-NMR (400 MHz, DMSO-d6) δ [ppm]: 1.363 (0.51), 1.401 (0.38), 1.417 (0.32), 1.834 (0.20), 1.850 (0.25), 2.327 (0.33), 2.670 (0.34), 3.038 (0.33), 3.053 (0.32), 3.982 (1.18), 4.230 (0.29), 4.820 (0.61), 4.838 (0.59), 7.084 (1.87), 7.436 (0.29), 7.448 (0.28), 7.499 (0.23), 7.509 (0.23), 7.520 (0.23), 7.531 (0.21), 7.678 (0.30), 7.695 (0.41), 7.706 (0.56), 7.728 (0.58), 7.837 (0.23), 7.856 (0.28), 7.858 (0.28), 7.877 (0.22), 7.906 (0.61), 7.928 (0.46), 8.073 (0.29), 8.094 (0.37), 8.104 (0.30), 8.118 (0.45), 8.138 (0.32), 8.156 (0.73), 8.500 (0.33), 8.512 (0.29), 8.617 (0.52), 8.643 (0.45), 8.935 (0.30), 8.939 (0.28), 8.945 (0.28).

### Example 31C

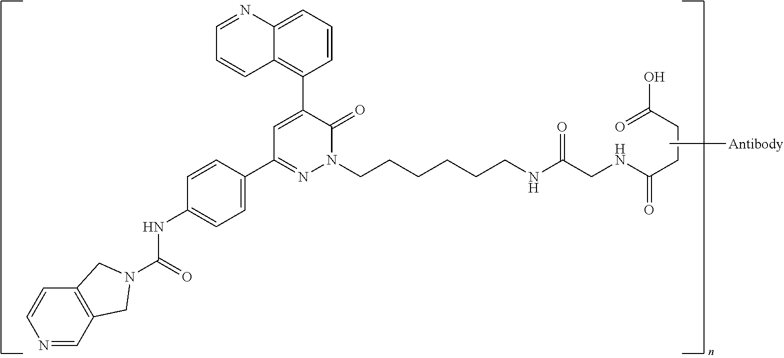

5 mg of anti-B7H3 TPP-8382 (c=15.1 mg/mL) were coupled with Final Intermediate 31-1 N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide (210 μg, 90% purity, 0.27 μmol) according to procedure 2 and the reaction was, after Sephadex purification, concentrated by ultracentrifugation and rediluted with PBS.

Protein concentration: 2.20 mg/mL

Drug/mAb ratio: 3.3 (UV)

### Example 31D

5 mg of anti-C4.4a TPP-509 (c=9.87 mg/mL) were coupled with Final Intermediate 31-1 N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide (210 μg, 90% purity, 0.27 μmol) according procedure 2 and the reaction was, after Sephadex purification, concentrated by ultracentrifugation and rediluted with PBS.

Protein concentration: 1.84 mg/mL

Drug/mAb ratio: 2.5 (UV)

### Example 31E

5 mg of anti-C4.4a TPP-668 (c=11.62 mg/mL) were coupled with Final Intermediate 31-1 N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide (210 μg, 90% purity, 0.27 μmol) according procedure 2 and the reaction was, after Sephadex purification, concentrated by ultracentrifugation and rediluted with PBS.

Protein concentration: 2.40 mg/mL

Drug/mAb ratio: 2.5 (UV)

### Intermediate 31-2

### S-{1-[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]-2,5-dioxopyrrolidin-3-yl}-N-{[2-(trimethylsilyl)ethoxy]carbonyl}-L-cysteine

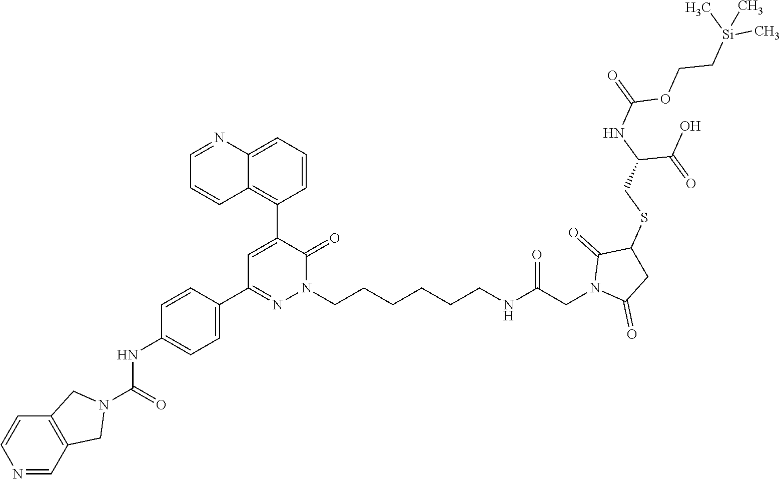 [SMILES: C[Si](C)(C)CCOC(=O)N[C@@H](CSC1CC(=O)N(CC(=O)NCCCCCCn2nc(-c3ccc(NC(=O)N4Cc5ccncc5C4)cc3)cc(-c3cccc4ncccc34)c2=O)C1=O)C(=O)O]

To a solution of N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide (see Final Intermediate 31-1, (10.0 mg, 13.6 μmol) in DMF (2.6 mL) was added N-{[2-(trimethylsilyl)ethoxy]carbonyl}-L-cysteine (see Intermediate 30-2, 7.24 mg, 27.3 μmol) and the mixture was stirred for 4 h at r.t. and 3 days at 6° C. Then to the reaction solution toluene was added and the mixture was concentrated under reduced pressure to give 13.0 mg (90% purity, 89% yield) of the crude produce which was directly used in the next step.

LC-MS (Method 2): Rt=0.83 min, MS (ESIpos): m/z=962 [M+H]⁺ MS (ESIneg): m/z=960 [M−H]⁻

### Intermediate 31-3

### 2 or 3-{[(2R)-2-carboxy-2-({[2-(trimethylsilyl)ethoxy]carbonyl}amino)ethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]amino}-4-oxobutanoic Acid

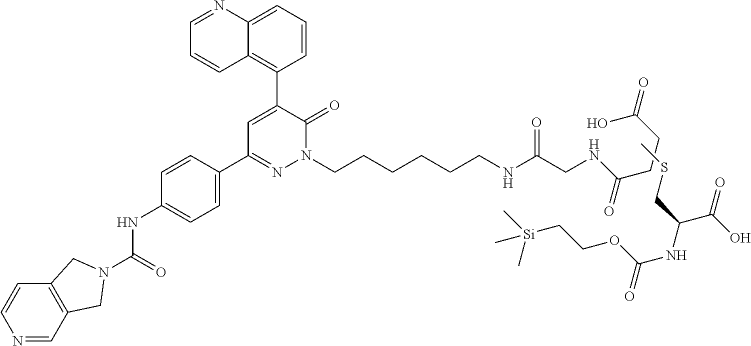

To S-{1-[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]-2,5-dioxopyrrolidin-3-yl}-N-{[2-(trimethylsilyl)ethoxy]carbonyl}-L-cysteine (see crude Intermediate 31-2, 13.0 mg, 13.5 μmol) in tetrahydrofuran/water (1:1, 1.8 mL) was added lithium hydroxide (1.29 mg, 54.0 μmol) in water (850 μL) and the mixture was stirred at r.t. for 2 h. The addition of added lithium hydroxide (1.29 mg, 54.0 μmol) in water (850 μL) was repeated and stirring was continued for 1 h. The mixture was neutralized with formic acid (4 μl, 108 μmol) and purified by preparative HPLC to give to give 7.0 mg (90% purity, 48% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-ODS-AQ 10 μm 280×51 mm; Eluent A: water+0.1% formic acid; Eluent B: acetonitrile; gradient: 0-22 min 30-80% B; rate 150 mL/min, temperature 25° C.

LC-MS (Method 1): Rt=0.96 min; MS (ESIneg): m/z=978 [M−H]⁻

¹H-NMR (400 MHz, DMSO-d6) δ [ppm]: 0.000 (4.96), 0.016 (0.97), 0.883 (0.32), 0.902 (0.38), 0.925 (0.32), 1.229 (0.75), 1.363 (0.43), 1.847 (0.27), 2.070 (0.27), 2.514 (16.00), 2.519 (11.26), 2.683 (0.22), 3.051 (0.27), 3.501 (0.32), 3.609 (0.22), 3.990 (0.32), 4.011 (0.38), 4.030 (0.32), 4.224 (0.27), 4.821 (0.54), 4.840 (0.59), 7.432 (0.27), 7.444 (0.27), 7.503 (0.27), 7.513 (0.27), 7.524 (0.27), 7.534 (0.27), 7.675 (0.32), 7.693 (0.38), 7.712 (0.48), 7.735 (0.65), 7.833 (0.38), 7.854 (0.43), 7.873 (0.32), 7.898 (0.75), 7.920 (0.59), 8.072 (0.32), 8.092 (0.27), 8.113 (0.43), 8.134 (0.32), 8.149 (1.02), 8.494 (0.22), 8.613 (0.38), 8.936 (0.32), 8.942 (0.32).

### Example 31M

### 2 or 3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]amino}-4-oxobutanoic Acid

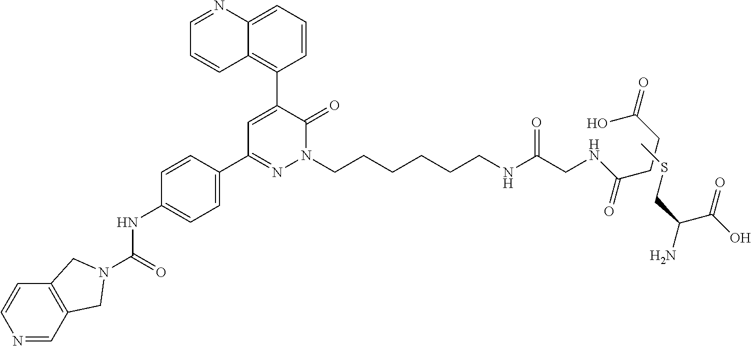

To 2 or 3-{[(2R)-2-carboxy-2-({[2-(trimethylsilyl)ethoxy]carbonyl}amino)ethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]amino}-4-oxobutanoic acid (see Intermediate 31-3, 6.00 mg, 6.12 μmol) in 2,2,2-trifluoroethanol (850 μL) was added anhydrous zinc chloride (3.34 ma. 24.5 μmol) and the suspension was stirred at 50° C. for 4 h followed by 20 h at r.t. After additional 4 h at 50° C. the mixture was cooled to r.t. and 2,2′,2″,2′″-(ethane-1,2-diyldinitrilo)tetraacetic acid (7.16 mg, 24.5 μmol), aqueous formic acid (0.1%, 1 mL) and dimethyl sulfoxide were added and the mixture was purified by preparative HPLC to give to give 1.79 mg of the title compound (90% purity, 31% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: YMC-Actus-ODS-AQ-HG 10 μm 150×20 mm. Eluent A: water+0.1% formic acid; Eluent B: acetonitrile; gradient: 0-14 min 1-30% B, 14-20 min 30% B; rate 60 mL/min, temperature 25° C.

LC-MS (Method 1): Rt=0.61 min; MS (ESIneg): m/z=834 [M−H]⁻

¹H-NMR (400 MHz, DMSO-d6) δ [ppm]: 0.882 (0.49), 0.898 (0.49), 1.232 (1.24), 1.366 (1.20), 1.407 (0.83), 1.425 (0.79), 1.833 (0.56), 1.850 (0.75), 2.072 (14.61), 2.457 (0.49), 2.518 (16.00), 2.522 (9.88), 2.539 (1.84), 2.660 (0.71), 3.059 (1.05), 3.077 (1.16), 3.091 (1.01), 3.622 (1.13), 3.638 (1.46), 3.681 (0.71), 3.695 (0.68), 3.704 (0.60), 4.229 (0.79), 4.826 (1.69), 4.844 (1.73), 7.435 (0.94), 7.448 (0.98), 7.506 (0.79), 7.516 (0.83), 7.527 (0.83), 7.537 (0.83), 7.678 (0.94), 7.693 (1.09), 7.711 (1.58), 7.733 (1.80), 7.839 (1.05), 7.857 (0.94), 7.860 (1.20), 7.878 (0.94), 7.902 (2.25), 7.924 (1.77), 8.073 (0.94), 8.093 (0.83), 8.119 (1.13), 8.140 (0.98), 8.157 (2.25), 8.499 (1.09), 8.511 (1.01), 8.616 (1.69), 8.692 (0.64), 8.697 (0.64), 8.938 (0.98), 8.941 (1.09), 8.948 (0.98), 8.952 (0.94).

### Final Intermediate 32-1

### N-{4-[1-(4-{[6-(2,5-dioxo-2,5-dihydro-1H-pyrrol-1-yl)hexanoyl]amino}butyl)-6-oxo-5-(quinolin-7-yl)-1,6-dihydropyridazin-3-yl]phenyl}-1,3-dihydro-2H-pyrrolo[3,4-c]pyridine-2-carboxamide

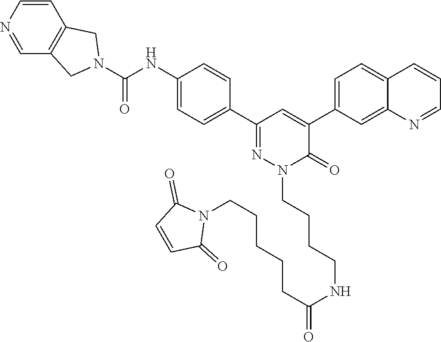 [SMILES: O=C(CCCCCN1C(=O)C=CC1=O)NCCCCn1nc(-c2ccc(NC(=O)N3Cc4ccncc4C3)cc2)cc(-c2ccc3cccnc3c2)c1=O]

A mixture of N-{4-[1-(4-aminobutyl)-6-oxo-5-(quinolin-7-yl)-1,6-dihydropyridazin-3-yl]phenyl}-1,3-dihydro-2H-pyrrolo[3,4-c]pyridine-2-carboxamide (see Example 25, 30.0 mg, 56.4 μmol), 1-{6-[(2,5-dioxopyrrolidin-1-yl)oxy]-6-oxohexyl}-1H-pyrrole-2,5-dione (20.9 mg, 67.7 μmol) and N,N-diisopropylethylamine (20 μl, 110 μmol) in DMF (870 μl) was stirred at r.t. for 2 h.

Then to the reaction solution toluene and formic acid (4.3 μl, 110 μmol) were added and the mixture was concentrated under vacuum. The crude product was purified by Isolera (4 g silicagel, eluent: dichloromethane/isopropyl alcohol, gradient) to give 11.6 mg of the desired product (80% purity, 23% yield).

LC-MS (Method 2): Rt=0.98 min, MS (ESIpos): m/z=725 [M+H]⁺

¹H-NMR (400 MHz, DMSO-d6) δ [ppm]: 1.147 (1.52), 1.164 (1.24), 1.185 (0.83), 1.208 (0.83), 1.231 (3.03), 1.256 (1.47), 1.295 (0.78), 1.414 (1.38), 1.442 (2.07), 1.461 (2.34), 1.479 (2.11), 1.830 (1.01), 1.847 (1.15), 1.995 (1.47), 2.013 (2.39), 2.031 (1.20), 2.326 (2.76), 2.590 (0.78), 2.665 (2.11), 2.668 (2.76), 3.083 (0.74), 3.100 (1.56), 3.115 (1.52), 3.304 (2.48), 4.252 (1.06), 4.268 (1.79), 4.286 (0.97), 4.832 (2.94), 4.851 (2.85), 6.972 (8.64), 7.443 (1.43), 7.455 (1.43), 7.581 (1.20), 7.592 (1.29), 7.602 (1.24), 7.612 (1.24), 7.730 (2.57), 7.751 (2.80), 7.797 (1.20), 7.962 (2.90), 7.984 (2.39), 8.057 (1.33), 8.079 (2.11), 8.135 (1.61), 8.139 (1.52), 8.161 (0.92), 8.325 (3.31), 8.416 (1.29), 8.435 (1.20), 8.505 (1.75), 8.517 (1.70), 8.624 (2.71), 8.652 (2.16), 8.698 (2.25), 8.968 (1.56), 8.974 (1.47), 8.978 (1.47).

### Example 32C

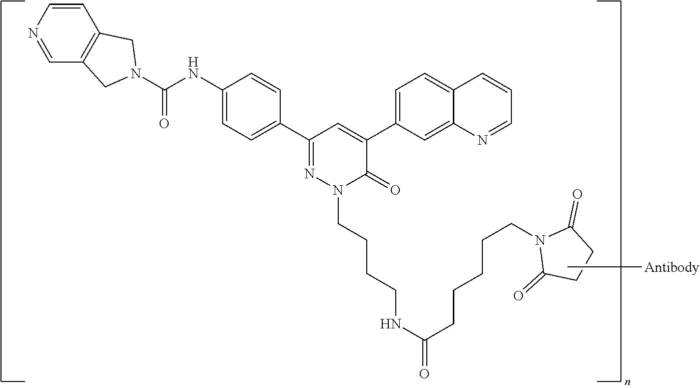

5 mg of anti-B7H3 TPP-8382 (15.1 mg/mL) were coupled with Final Intermediate 32-1 N-{4-[1-(4-{[6-(2,5-dioxo-2,5-dihydro-1H-pyrrol-1-yl)hexanoyl]amino}butyl)-6-oxo-5-(quinolin-7-yl)-1,6-dihydropyridazin-3-yl]phenyl}-1,3-dihydro-2H-pyrrolo[3,4-c]pyridine-2-carboxamide (240 μg, 80% purity, 0.27 μmol) according procedure 1 and the reaction was, after Sephadex purification, concentrated by ultracentrifugation and rediluted with PBS.

Protein concentration: 2.27 mg/mL

Drug/mAb ratio: 1.0 (LC-MS)

### Example 32E

5 mg of anti-C4.4a TPP-668 (11.62 mg/mL) were coupled with Final Intermediate 32-1 N-{4-[1-(4-{[6-(2,5-dioxo-2,5-dihydro-1H-pyrrol-1-yl)hexanoyl]amino}butyl)-6-oxo-5-(quinolin-7-yl)-1,6-dihydropyridazin-3-yl]phenyl}-1,3-dihydro-2H-pyrrolo[3,4-c]pyridine-2-carboxamide (240 μg, 80% purity, 0.27 μmol) according procedure 1 and the reaction was, after Sephadex purification, concentrated by ultracentrifugation and rediluted with PBS.

Protein concentration: 2.33 mg/mL

Drug/mAb ratio: 0.4 (LC-MS)

### Final Intermediate 33-1

### N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide

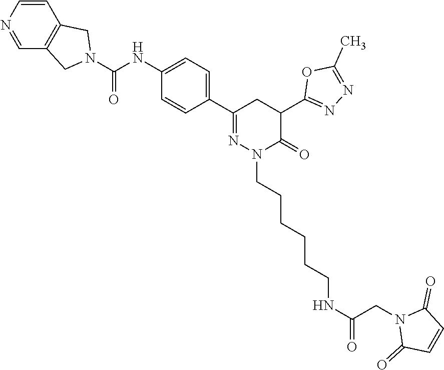 [SMILES: Cc1nnc(C2CC(c3ccc(NC(=O)N4Cc5ccncc5C4)cc3)=NN(CCCCCCNC(=O)CN3C(=O)C=CC3=O)C2=O)o1]

A mixture of N-{4-[1-(6-aminohexyl)-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide (see Example 11, 7.60 mg, 14.7 μmol), maleimido acetic acid N-hydroxysuccinimide ester (3.71 mg, 14.7 μmol) and N,N-diisopropylethylamine (5.1 μl, 29 μmol) in DMF (280 μl) was stirred at r.t. for 30 min. Then formic acid (1.1 μl, 29 μmol) was added to the reaction solution and the mixture was concentrated under vacuum. The crude product was purified by column chromatography (SiO₂, eluent: dichloromethane/isopropyl alcohol) to give 2.2 mg of the desired product (80% purity, 18% yield).

LC-MS (Method 1): Rt=0.75 min; MS (ESIpos): m/z=654 [M+H]⁺

¹H-NMR (400 MHz, DMSO-d₆) δ [ppm]: 1.026 (8.25), 1.040 (8.13), 1.233 (16.00), 1.272 (14.10), 1.349 (7.75), 1.474 (5.46), 1.629 (4.83), 2.326 (9.65), 2.625 (11.05), 2.668 (10.67), 3.029 (7.24), 3.465 (3.30), 3.751 (6.10), 3.982 (14.60), 4.834 (12.95), 5.759 (5.33), 7.083 (15.11), 7.446 (4.83), 7.691 (8.00), 7.735 (9.65), 7.756 (6.98), 7.876 (2.54), 8.092 (3.94), 8.511 (6.48), 8.618 (6.22), 8.665 (5.46).

### Example 33C

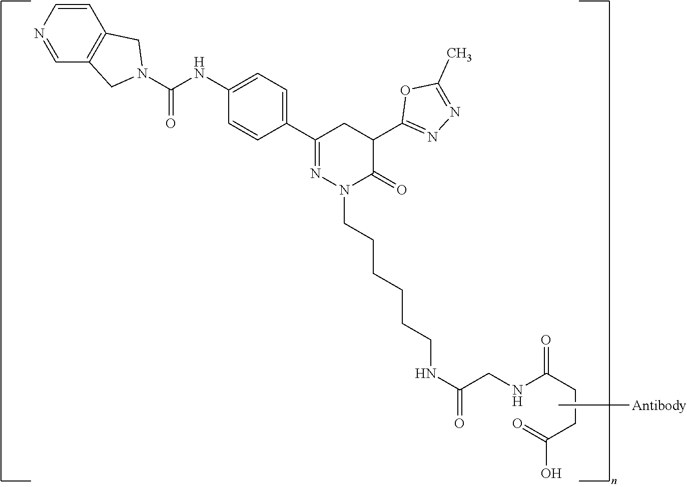

5 mg of anti-B7H3 TPP-8382 (c=15.1 mg/mL) were coupled with Final Intermediate 33-1 N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide (220 μg, 80% purity, 0.27 μmol) according procedure 2 and the reaction was, after Sephadex purification, concentrated by ultracentrifugation and rediluted with PBS.

Protein concentration: 1.86 mg/mL

Drug/mAb ratio: 4.8 (UV)

### Final Intermediate 34-1

### N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide

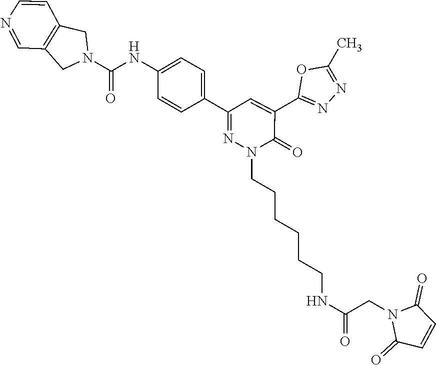 [SMILES: Cc1nnc(-c2cc(-c3ccc(NC(=O)N4Cc5ccncc5C4)cc3)nn(CCCCCCNC(=O)CN3C(=O)C=CC3=O)c2=O)o1]

A mixture of N-{4-[1-(6-aminohexyl)-5-(5-methyl-1,3,4-oxadiazol-2-yl)-6-oxo-1,6-dihydro-pyridazin-3-yl]phenyl}-1,3-dihydro-2H-pyrrolo[3,4-c]pyridine-2-carboxamide (see Example 10, 38.7 mg, 75.2 μmol), maleimido acetic acid N-hydroxysuccinimide ester (19.0 mg, 75.2 μmol) and N,N-diisopropylethylamine (26 μl, 150 μmol) in DMF (1.4 mL) was stirred at r.t. for 30 min. Then formic acid (5.7 μl, 150 μmol) was added to the reaction solution and the mixture was concentrated under vacuum. The crude product was purified by Isolera (4 g silicagel, (eluent: dichloromethane/isopropyl alcohol) to give 19.4 mg (93% purity, 37% yield) of the desired product.

LC-MS (Method 1): Rt=0.70 min; MS (ESIneg): m/z=650 [M−H]⁻

¹H-NMR (400 MHz, DMSO-d6) δ [ppm]: 1.026 (15.89), 1.042 (16.00), 1.239 (2.40), 1.255 (2.11), 1.269 (1.42), 1.345 (2.29), 1.387 (1.05), 1.401 (1.11), 1.418 (0.69), 1.799 (0.76), 1.817 (1.00), 1.832 (0.69), 2.331 (1.08), 2.518 (5.67), 2.523 (3.80), 2.627 (15.60), 2.673 (1.13), 3.013 (0.63), 3.030 (1.48), 3.044 (1.48), 3.060 (0.55), 3.984 (7.06), 4.213 (0.98), 4.231 (1.58), 4.248 (0.92), 4.830 (2.43), 4.847 (2.40), 7.082 (12.73), 7.135 (0.47), 7.443 (1.29), 7.456 (1.29), 7.735 (2.77), 7.757 (3.43), 7.875 (3.64), 7.897 (2.56), 8.087 (0.53), 8.101 (1.03), 8.113 (0.53), 8.505 (1.56), 8.511 (5.38), 8.623 (2.03), 8.682 (2.37).

### Example 34Ca

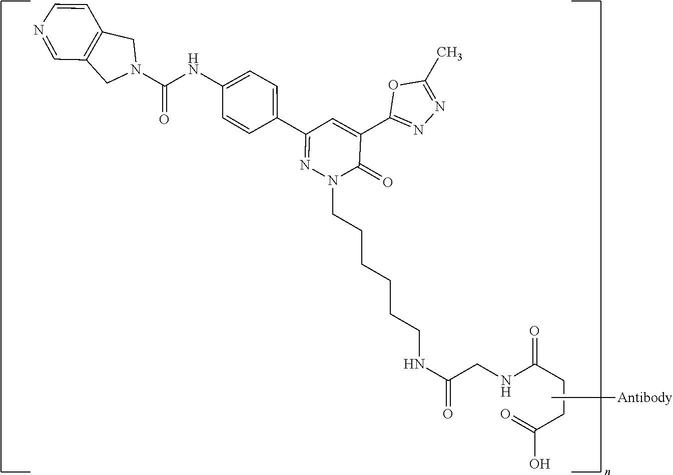

5 mg of anti-B7H3 TPP-8382 (c=15.1 mg/mL) were coupled with Final Intermediate 34-1 N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (220 μg, 80% purity, 0.27 μmol) according procedure 2 and the reaction was, after Sephadex purification, concentrated by ultracentrifugation and rediluted with PBS.

Protein concentration: 0.67 mg/mL

Drug/mAb ratio: 4.0 (UV)

### Example 34Cb

5 mg of anti-B7H3 TPP-8382 (c=15.1 mg/mL) were coupled with Final Intermediate 34-1 N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (430 μg, 80% purity, 0.53 μmol) according procedure 2 and the reaction was, after Sephadex purification, concentrated by ultracentrifugation and rediluted with PBS.

Protein concentration: 0.99 mg/mL

Drug/mAb ratio: 8.4 (UV)

### Example 34 Da

5 mg of anti-C4.4a TPP-509 (c=9.87 mg/mL) were coupled with Final Intermediate 34-1 N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (220 μg, 80% purity, 0.27 μmol) according procedure 2 and the reaction was, after Sephadex purification, concentrated by ultracentrifugation and rediluted with PBS.

Protein concentration: 2.35 mg/mL

Drug/mAb ratio: 3.4 (UV)

### Example 34Db

5 mg of anti-C4.4a TPP-509 (c=9.87 mg/mL) were coupled with Final Intermediate 34-1N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (430 μg, 80% purity, 0.53 μmol) according procedure 2 and the reaction was, after Sephadex purification, concentrated by ultracentrifugation and rediluted with PBS.

Protein concentration: 2.44 mg/mL

Drug/mAb ratio: 6.4 (UV)

### Example 34Ea

5 mg of anti-C4.4a TPP-668 (c=11.62 mg/mL) were coupled with Final Intermediate 34-1 N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (220 μg, 80% purity, 0.27 μmol) according procedure 2 and the reaction was, after Sephadex purification, concentrated by ultracentrifugation and rediluted with PBS.

Protein concentration: 2.70 mg/mL

Drug/mAb ratio: 3.3 (UV)

### Example 34Eb

5 mg of anti-C4.4a TPP-668 (c=11.62 mg/mL) were coupled with Final Intermediate 34-1 N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (430 μg, 80% purity, 0.53 μmol) according procedure 2 and the reaction was, after Sephadex purification, concentrated by ultracentrifugation and rediluted with PBS.

Protein concentration: 2.96 mg/mL

Drug/mAb ratio: 6.4 (UV)

### Intermediate 34-2

### S-{1-[2-({6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}amino)-2-oxoethyl]-2,5-dioxopyrrolidin-3-yl}-N-{[2-(trimethylsilyl)ethoxy]carbonyl}-L-cysteine

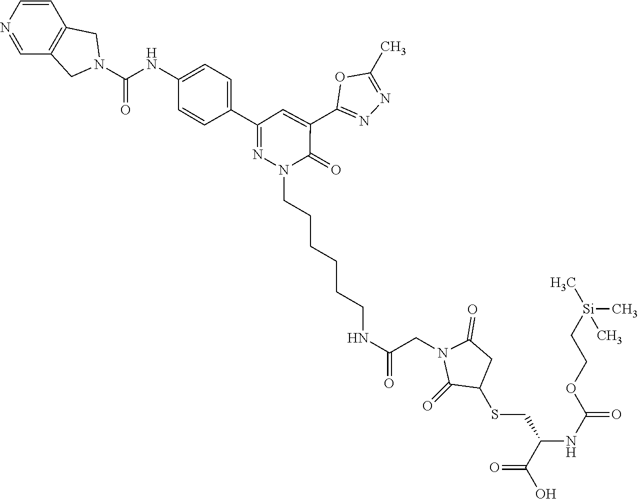 [SMILES: Cc1nnc(-c2cc(-c3ccc(NC(=O)N4Cc5ccncc5C4)cc3)nn(CCCCCCNC(=O)CN3C(=O)CC(SC[C@H](NC(=O)OCC[Si](C)(C)C)C(=O)O)C3=O)c2=O)o1]

To a solution of N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide (see Final Intermediate 34-1, 12.5 mg, 19.2 μmol) in DMF (3.7 mL) was added N-{[2-(trimethylsilyl)ethoxy]carbonyl}-L-cysteine (see Intermediate 30-2, 10.2 mg, 38.4 μmol) and the mixture was stirred for 23 h at r.t. Then to the reaction solution toluene was added and the mixture was concentrated under reduced pressure (two times azeotropic distillation with toluene was executed) to give 23 mg of the title compound as crude product (77% purity, 99% yield), which was directly used in the next step.

### Intermediate 34-3

### 2 or 3-{[(2R)-2-carboxy-2-({[2-(trimethylsilyl)ethoxy]carbonyl}amino)ethyl]sulfanyl}-4-{[2-({6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}amino)-2-oxoethyl]amino}-4-oxobutanoic Acid

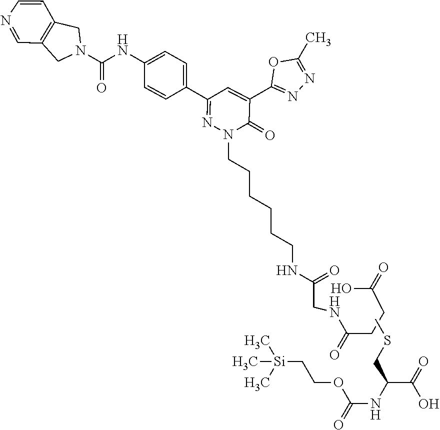

To S-{1-[2-({6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}amino)-2-oxoethyl]-2,5-dioxopyrrolidin-3-yl}-N-{[2-(trimethylsilyl)ethoxy]carbonyl}-L-cysteine (see Intermediate 34-2, 22.7 mg, 77% purity, 19.1 μmol) in tetrahydrofuran/water (1:1, 2.5 mL) was added aqueous lithium hydroxide solution (38 μl, 4.0 M, 150 μmol) and the mixture was stirred at r.t. for 2.5 h. The mixture was concentrated under reduced pressure purified by preparative HPLC to give to give 7.7 mg of the title compound (99% purity, 43% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18 10 μm 125×30 mm, Eluent A: water+0.1% formic acid; Eluent B: acetonitrile; gradient: 0-6 min 15-55% B, 6-8 min 55-100% B, rate 150 mL/min, temperature 25° C.

LC-MS (Method 1): Rt=0.92 min: MS (ESIneg): m/z=933 [M−H]⁻

### Example 34M

### 2 or 3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}amino)-2-oxoethyl]amino}-4-oxobutanoic Acid

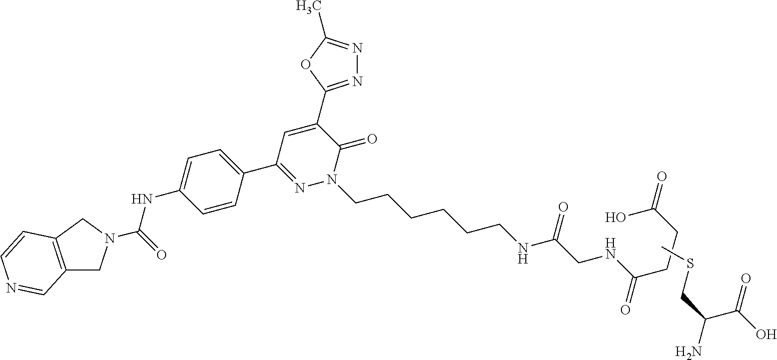

To 2 or 3-{[(2R)-2-carboxy-2-({[2-(trimethylsilyl)ethoxy]carbonyl}amino)ethyl]sulfanyl}-4-{[2-({6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}amino)-2-oxoethyl]amino}-4-oxobutanoic acid (see Intermediate 34-3, 7.70 mg, 8.23 μmol) in 2,2,2-trifluoroethanol (1.1 mL) anhydrous zinc chloride (4.49 mg, 32.9 μmol) was added and stirred at 50° C. for 3 h. Then the mixture was cooled down to r.t., ethylenedinitrilotetraacetic acid (10 mg), formic acid (0.1%, 1 mL) and Dimethyl sulfoxide were added and the mixture was purified by preparative HPLC to give to give 2.3 mg of the title compound (90% purity, 32% yield).

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG-10 μm 12 nm 150×20 mm, Eluent A: water+0.1% formic acid; Eluent B: acetonitrile; gradient: 0-6 min 10-50% B, 6-8 min 50-100% B, rate 150 mL/min, temperature 25° C.

LC-MS (Method 1): Rt=0.61 min; MS (ESIneg): m/z=789 [M−H]⁻

¹H-NMR (400 MHz, DMSO-d6) δ [ppm]: 1.229 (0.26), 1.341 (0.43), 1.394 (0.19), 1.411 (0.21), 1.477 (0.97), 1.813 (0.21), 2.322 (0.19), 2.327 (0.25), 2.522 (0.82), 2.539 (16.00), 2.625 (2.41), 2.665 (0.23), 2.669 (0.28), 2.673 (0.25), 3.050 (0.36), 3.063 (0.32), 3.598 (0.41), 3.627 (0.31), 3.643 (0.31), 3.668 (0.25), 3.683 (0.22), 4.210 (0.20), 4.228 (0.31), 4.246 (0.18), 4.837 (0.46), 4.852 (0.46), 7.438 (0.25), 7.450 (0.25), 7.745 (0.36), 7.766 (0.48), 7.866 (0.65), 7.889 (0.41), 8.498 (0.36), 8.506 (0.82), 8.616 (0.43), 8.780 (0.17).

### Intermediate 35-1

### N²-{[(9H-Fluoren-9-yl)methoxy]carbonyl}-N⁶-(26-oxo-2,5,8,11,14,17,20,23-octaoxahexacosan-26-yl)-L-lysine

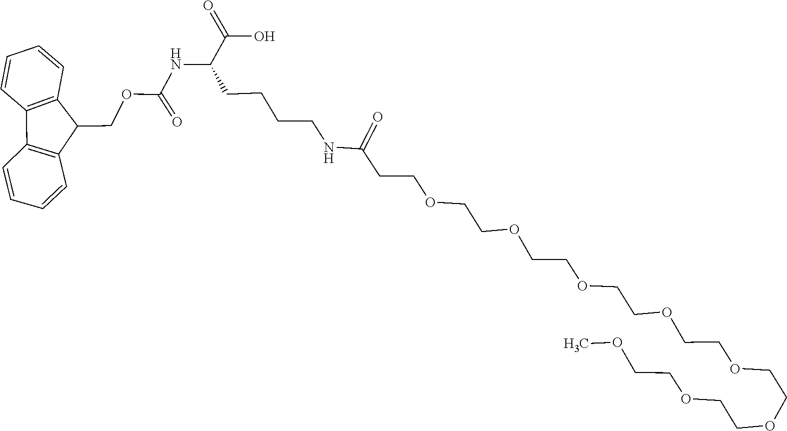 [SMILES: COCCOCCOCCOCCOCCOCCOCCOCCC(=O)NCCCC[C@H](NC(=O)OCC1c2ccccc2-c2ccccc21)C(=O)O]

To 1-[(26-oxo-2,5,8,11,14,17,20,23-octaoxahexacosan-26-yl)oxy]pyrrolidine-2,5-dione (CAS 756525-90-3, 500 mg, 981 μmol) in DMF 8.6 mL) was added N2-[(9H-fluoren-9-ylmethoxy)carbonyl]-L-lysine (CAS 105047-45-8, 350 mg, 1.08 mmol) and N,N-diisopropylethylamine (600 μl, 3.4 mmol) The mixture was stirred for 1 h at r.t., then diluted with formic acid (130 μl, 3.4 mmol) in DMSO (9 mL) and purified by preparative HPLC to give to give 560 mg (90% purity, 67% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-8 min 5-60% B, 8-13 min 60% B, rate 150 mL/min, temperature 25° C.

LC-MS (Method 1): Rt=1.10 min; MS (ESIpos): m/z=763 [M+H]⁺.

¹H-NMR (400 MHz, DMSO-d₆) δ [ppm]: 1.25-1.43 (m, 4H), 1.53-1.74 (m, 2H), 2.26-2.32 (m, 2H), 2.98-3.06 (m, 2H), 3.23 (s, 3H), 3.40-3.44 (m, 2H), 3.44-3.51 (m, 26H), 3.57 (t, 2H), 3.84-3.95 (m, 1H), 4.19-4.29 (m, 3H), 7.33 (t, 2H), 7.42 (t, 2H), 7.62 (d, 1H), 7.73 (d, 2H), 7.79-7.85 (m, 1H), 7.90 (d, 2H).

### Intermediate 35-2

### (9H-fluoren-9-yl)methyl {(32S)-40-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]-26,33-dioxo-2,5,8,11,14,17,20,23-octaoxa-27,34-diazatetracontan-32-yl}carbamate

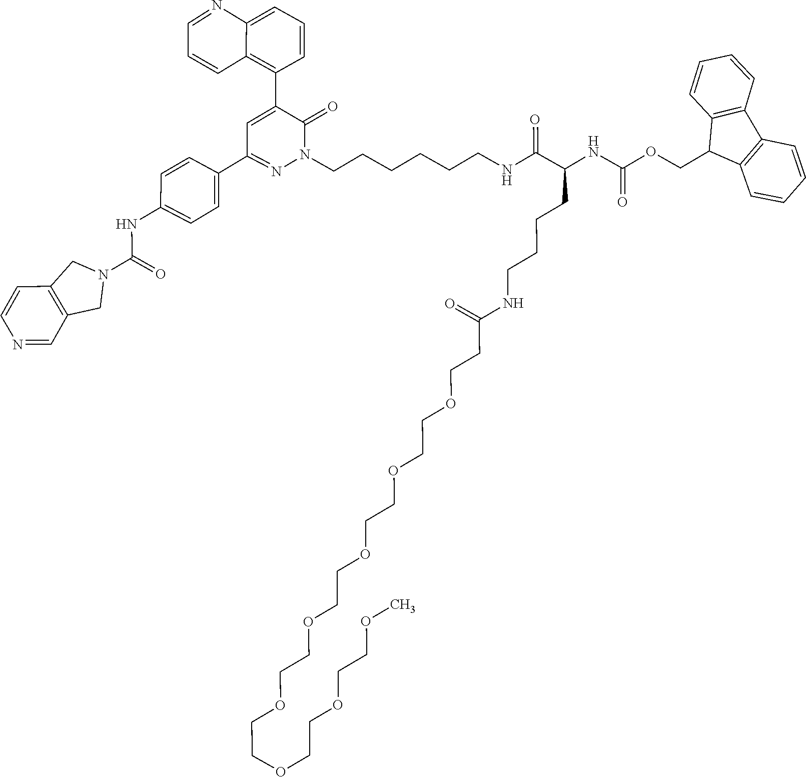 [SMILES: COCCOCCOCCOCCOCCOCCOCCOCCC(=O)NCCCC[C@H](NC(=O)OCC1c2ccccc2-c2ccccc21)C(=O)NCCCCCCn1nc(-c2ccc(NC(=O)N3Cc4ccncc4C3)cc2)cc(-c2cccc3ncccc23)c1=O]

To N2-{[(9H-fluoren-9-yl)methoxy]carbonyl}-N6-(26-oxo-2,5,8,11,14,17,20,23-octaoxahexacosan-26-yl)-L-lysine (149 mg, 195 μmol) in DMF (0.65 mL) wad added 4-methylmorpholin (41 μl, 380 μmol) and HATU (71.5 mg, 188 μmol) and the mixture was stirred for 20 min. at r.t. Then the mixture was added to a suspension of N-{4-[1-(6-aminohexyl)-6-oxo-5-(quinolin-5-yl)-1,6-dihydropyridazin-3-yl]phenyl}-1,3-dihydro-2H-pyrrolo[3,4-c]pyridine-2-carboxamide (78.0 mg, 139 μmol) in DMF (0.8 mL) and stirring was continued at r. t. for 30 min. Then formic acid (14 μl, 380 μmol) was added and the reaction mixture was diluted with DMSO. Purification by preparative HPLC yielded 102 mg (90% purity, 50% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rt=0.61 min; MS (ESIpos): m/z=653 [M+2H]²⁺, (ESIneg): m/z=1303 [M−H]⁻.

¹H-NMR (400 MHz, DMSO-d6) δ [ppm]: 1.231 (0.17), 1.356 (0.83), 1.399 (0.43), 1.415 (0.38), 1.555 (0.16), 1.831 (0.29), 1.851 (0.22), 2.247 (0.35), 2.263 (0.77), 2.279 (0.41), 2.518 (1.69), 2.523 (1.21), 2.995 (0.33), 3.034 (0.26), 3.050 (0.29), 3.065 (0.22), 3.219 (8.06), 3.229 (0.21), 3.231 (0.19), 3.393 (0.56), 3.403 (1.00), 3.410 (0.80), 3.417 (1.37), 3.444 (1.61), 3.448 (1.71), 3.459 (0.86), 3.467 (7.44), 3.475 (9.17), 3.481 (16.00), 3.497 (1.13), 3.499 (1.04), 3.540 (0.43), 3.556 (0.90), 3.572 (0.44), 3.885 (0.19), 3.899 (0.19), 4.188 (0.46), 4.203 (0.78), 4.228 (0.63), 4.237 (0.58), 4.256 (0.26), 4.814 (0.79), 4.833 (0.79), 7.280 (0.37), 7.298 (0.84), 7.316 (0.53), 7.374 (0.48), 7.393 (1.05), 7.413 (0.60), 7.432 (0.46), 7.445 (0.46), 7.487 (0.34), 7.497 (0.36), 7.509 (0.36), 7.519 (0.34), 7.665 (0.45), 7.680 (0.55), 7.691 (0.43), 7.706 (1.43), 7.729 (1.17), 7.775 (0.17), 7.789 (0.31), 7.803 (0.19), 7.829 (0.60), 7.837 (0.35), 7.847 (0.65), 7.850 (0.89), 7.856 (0.77), 7.868 (0.60), 7.876 (0.64), 7.900 (1.02), 7.922 (0.77), 8.059 (0.36), 8.079 (0.32), 8.112 (0.57), 8.133 (0.48), 8.149 (1.47), 8.499 (0.65), 8.511 (0.60), 8.614 (0.88), 8.640 (0.76), 8.926 (0.43), 8.929 (0.46), 8.936 (0.42), 8.939 (0.37).

### Intermediate 35-3

### N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,6-dihydropyridazin-3-yl]phenyl}-1,3-dihydro-2H-pyrrolo[3,4-c]pyridine-2-carboxamide

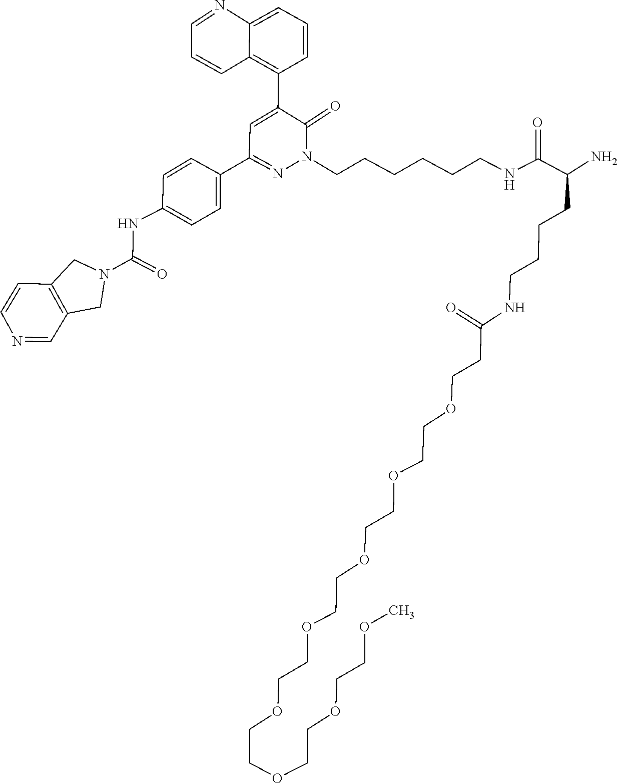 [SMILES: COCCOCCOCCOCCOCCOCCOCCOCCC(=O)NCCCC[C@H](N)C(=O)NCCCCCCn1nc(-c2ccc(NC(=O)N3Cc4ccncc4C3)cc2)cc(-c2cccc3ncccc23)c1=O]

To (9H-fluoren-9-yl)methyl {(32S)-40-[3-{4-[(1,3-dihydro-2H-pyrrolo[3,4-c]pyridine-2-carbonyl) amino]phenyl}-6-oxo-5-(quinolin-5-yl)pyridazin-1(6H)-yl]-26,33-dioxo-2,5,8,11,14,17,20,23-octaoxa-27,34-diazatetracontan-32-yl}carbamate (102 mg, 78.2 μmol) in DMF (510 μl) was added piperidine (130 μl, 1.3 mmol) and the mixture was stirred for 20 min at room temperature. Then formic acid (50 μl, 1.3 mmol) was added and the reaction mixture was diluted with DMSO. Purification by preparative HPLC yielded 57.4 mg (90% purity, 61% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-12 min 1-30% B, 12-14 min 30% B. 14-20 min 30-50% B; rate 150 mL/min, temperature 25° C.

LC-MS (Method 1): Rt=0.61 min; MS (ESIpos): m/z=542 [M+2H]²⁺, (ESIneg): m/z=1081 [M−H]⁻.

¹H-NMR (400 MHz, DMSO-d6) δ [ppm]: 1.237 (0.19), 1.256 (0.20), 1.270 (0.18), 1.325 (0.43), 1.344 (0.56), 1.366 (0.74), 1.415 (0.38), 1.432 (0.36), 1.450 (0.21), 1.835 (0.23), 1.852 (0.32), 1.869 (0.21), 2.247 (0.45), 2.263 (0.98), 2.280 (0.47), 2.331 (0.32), 2.518 (1.86), 2.523 (1.19), 2.962 (0.24), 2.979 (0.50), 2.993 (0.51), 3.010 (0.26), 3.034 (0.21), 3.051 (0.34), 3.065 (0.44), 3.079 (0.36), 3.095 (0.21), 3.124 (0.32), 3.141 (0.39), 3.155 (0.29), 3.225 (7.15), 3.312 (0.24), 3.399 (0.78), 3.410 (1.20), 3.416 (0.98), 3.423 (1.45), 3.434 (0.57), 3.441 (0.76), 3.448 (1.14), 3.452 (1.72), 3.458 (1.78), 3.462 (1.25), 3.469 (1.06), 3.478 (7.27), 3.489 (16.00), 3.504 (1.09), 3.540 (0.92), 3.557 (1.52), 3.573 (0.90), 3.652 (0.45), 4.227 (0.42), 4.821 (0.80), 4.839 (0.80), 7.436 (0.43), 7.448 (0.44), 7.502 (0.41), 7.512 (0.40), 7.523 (0.42), 7.533 (0.40), 7.675 (0.43), 7.678 (0.43), 7.693 (0.53), 7.695 (0.49), 7.708 (0.90), 7.730 (1.02), 7.773 (0.17), 7.787 (0.33), 7.800 (0.16), 7.837 (0.43), 7.855 (0.40), 7.859 (0.51), 7.877 (0.40), 7.889 (0.20), 7.902 (1.40), 7.925 (0.86), 8.077 (0.37), 8.096 (0.37), 8.118 (0.53), 8.139 (0.43), 8.158 (1.40), 8.342 (4.59), 8.500 (0.64), 8.512 (0.59), 8.617 (0.89), 8.668 (0.71), 8.935 (0.46), 8.939 (0.48), 8.946 (0.45), 8.950 (0.41).

### Final Intermediate 35-4

### N-(4-{1-[6-({N²-[(2,5-Dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,6-dihydropyridazin-3-yl}phenyl)-1,3-dihydro-2H-pyrrolo[3,4-c]pyridine-2-carboxamide

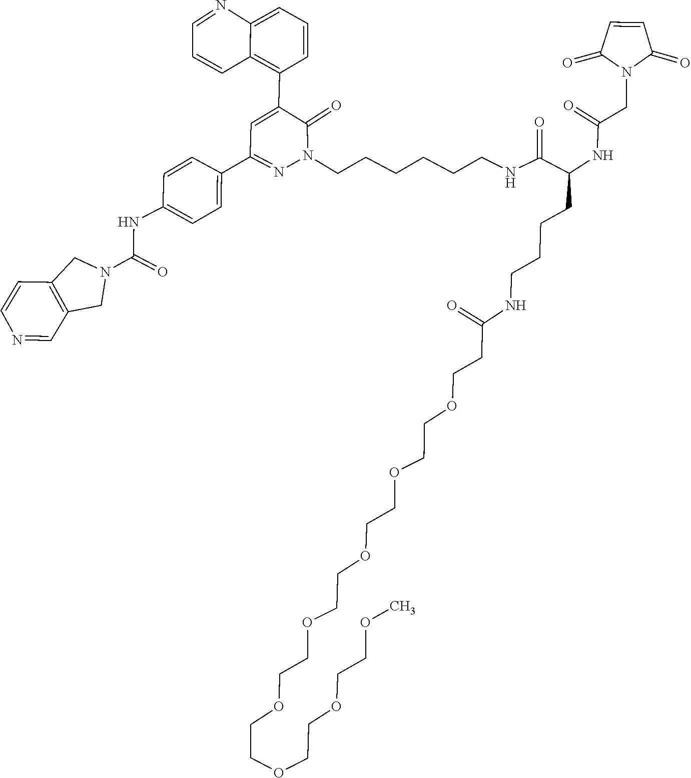 [SMILES: COCCOCCOCCOCCOCCOCCOCCOCCC(=O)NCCCC[C@H](NC(=O)CN1C(=O)C=CC1=O)C(=O)NCCCCCCn1nc(-c2ccc(NC(=O)N3Cc4ccncc4C3)cc2)cc(-c2cccc3ncccc23)c1=O]

A mixture of N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,6-dihydropyridazin-3-yl]phenyl}-1,3-dihydro-2H-pyrrolo[3,4-c]pyridine-2-carboxamide (see Intermediate 35-3, 30.0 mg, 26.3 μmol), maleimido acetic acid N-hydroxysuccinimide ester (7.30 mg, 29.0 μmol) and N,N-diisopropylethylamine (9.2 μl, 53 μmol) in DMF (0.55 mL) was stirred at r.t. for 30 min. Then the reaction was diluted with formic acid (2.0 μl, 53 μmol) in toluene (30 mL) and the mixture was concentrated under vacuum. The crude product was purified by column chromatography (SiO₂, dichloromethane/isopropyl alcohol, gradient containing 10% DMSO) to give 5.14 mg (90% purity, 14% yield) of the title compound.

LC-MS (Method 1): Rt=0.81 min; MS (ESIpos): m/z=610.8 [M+2H]²⁺, (ESIneg): m/z=1218 [M−H]⁻.

¹H-NMR (400 MHz, DMSO-d6) δ [ppm]: 1.036 (0.20), 1.046 (0.20), 1.051 (0.20), 1.213 (0.20), 1.231 (0.27), 1.360 (0.73), 1.402 (0.46), 1.419 (0.40), 1.847 (0.33), 2.250 (0.40), 2.266 (0.86), 2.282 (0.40), 2.331 (1.20), 2.518 (6.77), 2.523 (4.38), 2.539 (15.20), 2.673 (1.20), 2.964 (0.27), 2.979 (0.40), 2.993 (0.33), 3.008 (0.27), 3.024 (0.27), 3.053 (0.27), 3.067 (0.20), 3.224 (6.51), 3.381 (0.13), 3.400 (0.60), 3.409 (1.00), 3.416 (0.73), 3.423 (1.20), 3.452 (1.59), 3.456 (1.66), 3.476 (6.51), 3.488 (16.00), 3.503 (0.73), 3.541 (0.53), 3.557 (1.00), 3.574 (0.46), 4.028 (0.20), 4.070 (0.80), 4.085 (0.73), 4.127 (0.27), 4.146 (0.20), 4.160 (0.20), 4.226 (0.33), 4.819 (0.80), 4.837 (0.80), 7.065 (3.72), 7.436 (0.46), 7.448 (0.46), 7.498 (0.40), 7.509 (0.40), 7.519 (0.40), 7.530 (0.40), 7.676 (0.40), 7.695 (0.53), 7.704 (0.93), 7.726 (1.00), 7.753 (0.20), 7.768 (0.33), 7.837 (0.40), 7.858 (0.46), 7.876 (0.40), 7.902 (1.20), 7.925 (0.93), 8.075 (0.40), 8.094 (0.40), 8.116 (0.53), 8.138 (0.46), 8.155 (1.39), 8.288 (0.40), 8.309 (0.33), 8.500 (0.66), 8.512 (0.60), 8.617 (0.93), 8.643 (0.73), 8.932 (0.46), 8.937 (0.46), 8.943 (0.46), 8.947 (0.40).

### Example 35A

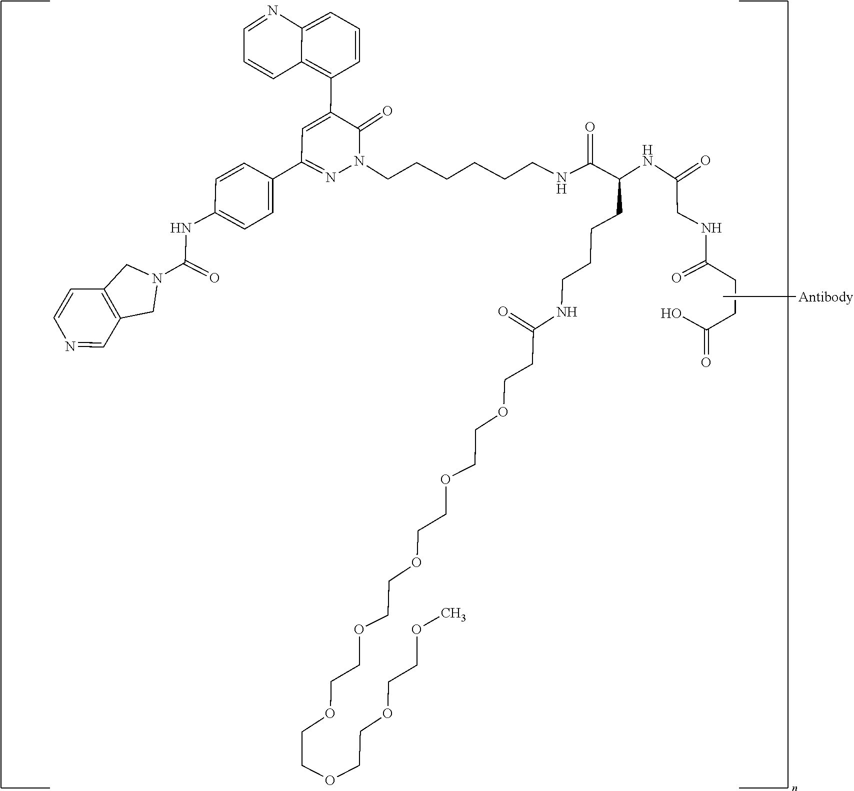

5 mg of anti-HER2 TPP-1015 (c=12.2 mg/mL) were coupled with Final Intermediate 35-4 N-(4-{1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,6-dihydropyridazin-3-yl}phenyl)-1,3-dihydro-2H-pyrrolo[3,4-c]pyridine-2-carboxamide (340 μg, 95% purity, 0.27 μmol) according procedure 2 and the reaction was, after Sephadex purification, concentrated by ultracentrifugation and rediluted with PBS.

Protein concentration: 0.82 mg/mL

Drug/mAb ratio: 5.6 (UV)

### Example 35C

5 mg of anti-B7H3 TPP-8382 (c=15.1 mg/mL) were coupled with Final Intermediate 35-4 N-(4-{1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,6-dihydropyridazin-3-yl}phenyl)-1,3-dihydro-2H-pyrrolo[3,4-c]pyridine-2-carboxamide (380 μg, 85% purity, 0.27 μmol) according procedure 2 and the reaction was, after Sephadex purification, concentrated by ultracentrifugation and rediluted with PBS.

Protein concentration: 1.5 mg/mL

Drug/mAb ratio: 2.0 (UV)

### Example 35D

5 mg of anti-C4.4a TPP-509 (c=9.87 mg/mL) were coupled with Final Intermediate 35-4 N-(4-{1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,6-dihydropyridazin-3-yl}phenyl)-1,3-dihydro-2H-pyrrolo[3,4-c]pyridine-2-carboxamide (380 μg, 85% purity, 0.27 μmol) according procedure 2 and the reaction was, after Sephadex purification, concentrated by ultracentrifugation and rediluted with PBS.

Protein concentration: 1.57 mg/mL

Drug/mAb ratio: 1.7 (UV)

### Intermediate 36-1

### (9H-fluoren-9-yl)methyl {(32S)-40-[(5S)-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-26,33-dioxo-2,5,8,11,14,17,20,23-octaoxa-27,34-diazatetracontan-32-yl}carbamate

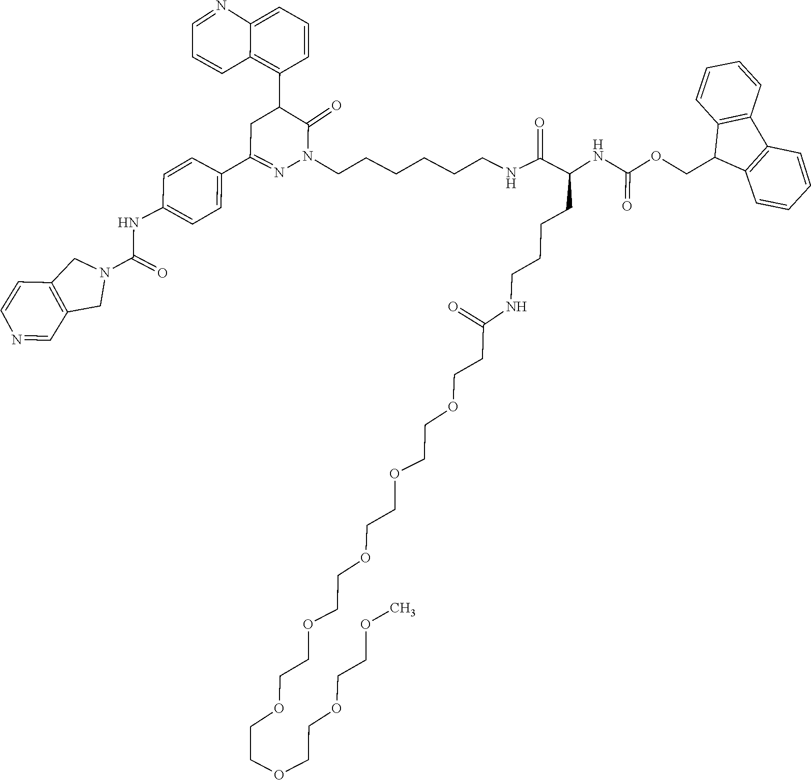 [SMILES: COCCOCCOCCOCCOCCOCCOCCOCCC(=O)NCCCC[C@H](NC(=O)OCC1c2ccccc2-c2ccccc21)C(=O)NCCCCCCN1N=C(c2ccc(NC(=O)N3Cc4ccncc4C3)cc2)CC(c2cccc3ncccc23)C1=O]

To N2-{[(9H-fluoren-9-yl)methoxy]carbonyl}-N6-(26-oxo-2,5,8,11,14,17,20,23-octaoxahexacosan-26-yl)-L-lysine (see Intermediate 35-1, 262 mg, 344 μmol) in DMF (1.0 mL) wad added 4-methylmorpholin (73 μl, 660 μmol) and HATU (126 mg, 332 μmol) and the mixture was stirred for 20 min. at r.t. Then the mixture was added to a suspension of N-{4-[1-(6-aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 3, 138 mg, 246 μmol) in DMF (1.5 mL) and stirring was continued at r. t. for 2 h, while after 1 h additional methylmorpholin (36 μl, 320 μmol) and HATU (32 mg, 84 μmol) were added. Then formic acid (25 μl, 660 μmol) was added and the reaction mixture was diluted with and DMSO. Purification by preparative HPLC yielded 112.4 mg (85% purity, 30% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rt=1.05 min; MS (ESipos): m/z=654 [M+2H]²⁺, (ESIneg): m/z=1305 [M−H]⁻.

¹H-NMR (400 MHz, DMSO-d6) δ [ppm]: 1.232 (0.32), 1.316 (0.81), 1.556 (0.18), 1.689 (0.28), 2.254 (0.35), 2.269 (0.70), 2.285 (0.39), 2.518 (8.54), 2.522 (5.64), 2.539 (7.32), 2.673 (1.37), 2.994 (0.46), 3.220 (7.25), 3.231 (0.42), 3.394 (0.74), 3.405 (1.05), 3.411 (0.98), 3.418 (1.30), 3.450 (1.82), 3.469 (6.72), 3.477 (8.19), 3.482 (16.00), 3.498 (1.75), 3.544 (0.46), 3.560 (0.91), 3.576 (0.53), 3.813 (0.28), 3.829 (0.28), 3.901 (0.18), 4.193 (0.35), 4.207 (0.53), 4.231 (0.42), 4.244 (0.49), 4.261 (0.25), 4.737 (0.18), 4.755 (0.21), 4.794 (0.77), 4.813 (0.77), 7.287 (0.35), 7.306 (0.77), 7.324 (0.53), 7.382 (0.53), 7.400 (0.91), 7.419 (0.88), 7.433 (0.81), 7.451 (0.39), 7.533 (0.32), 7.543 (0.32), 7.554 (0.32), 7.565 (0.32), 7.625 (0.74), 7.648 (1.12), 7.689 (0.35), 7.707 (1.54), 7.729 (1.16), 7.794 (0.32), 7.827 (0.32), 7.865 (0.74), 7.884 (0.70), 7.943 (0.49), 7.964 (0.42), 8.490 (0.56), 8.503 (0.56), 8.581 (0.32), 8.601 (1.12), 8.621 (0.74), 8.907 (0.42), 8.914 (0.42).

### Intermediate 36-2

### N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide

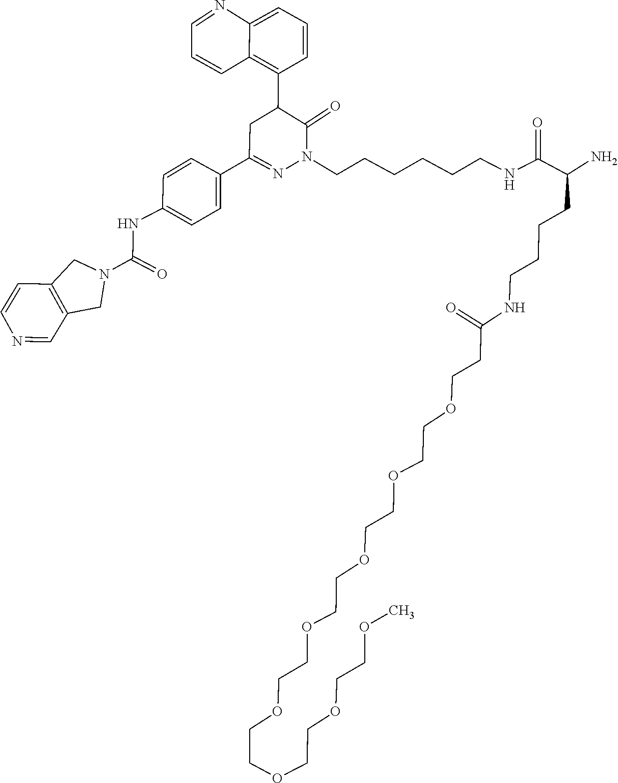 [SMILES: COCCOCCOCCOCCOCCOCCOCCOCCC(=O)NCCCC[C@H](N)C(=O)NCCCCCCN1N=C(c2ccc(NC(=O)N3Cc4ccncc4C3)cc2)CC(c2cccc3ncccc23)C1=O]

To (9H-fluoren-9-yl)methyl {(32S)-40-[(5S)-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-26,33-dioxo-2,5,8,11,14,17,20,23-octaoxa-27,34-diazatetracontan-32-yl}carbamate (112 mg, 85.7 μmol) in DMF (560 μL) was added piperidine (140 μl, 1.4 mmol) and the mixture was stirred for 20 min at room temperature. Then formic acid (55 μl, 1.5 mmol) was added and the reaction mixture was diluted with DMSO. Purification by preparative HPLC yielded 56.9 mg (80% purity, 49% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-12 min 1-30% B, 12-14 min 30% B. 14-20 min 30-50% B; rate 150 mL/min, temperature 25° C.

LC-MS (Method 1): Rt=0.70 min; MS (ESIpos): m/z=543 [M+2H]²⁺, (ESIneg): m/z=1083 [M−H]⁻.

¹H-NMR (400 MHz, DMSO-d6) δ [ppm]: 1.237 (0.21), 1.255 (0.23), 1.328 (1.20), 1.394 (0.38), 1.409 (0.40), 1.465 (0.17), 1.593 (0.31), 1.606 (0.38), 1.619 (0.25), 1.703 (0.36), 2.254 (0.48), 2.271 (0.99), 2.287 (0.49), 2.323 (0.31), 2.327 (0.40), 2.331 (0.30), 2.522 (1.73), 2.665 (0.31), 2.669 (0.40), 2.967 (0.23), 2.983 (0.52), 2.998 (0.53), 3.015 (0.25), 3.040 (0.33), 3.053 (0.42), 3.067 (0.32), 3.086 (0.18), 3.098 (0.25), 3.115 (0.31), 3.128 (0.21), 3.226 (5.88), 3.401 (1.37), 3.411 (1.65), 3.417 (1.21), 3.424 (1.72), 3.438 (0.65), 3.456 (1.95), 3.461 (2.00), 3.466 (1.54), 3.481 (7.53), 3.492 (16.00), 3.504 (1.16), 3.546 (0.62), 3.563 (1.17), 3.579 (0.56), 3.806 (0.20), 3.823 (0.37), 3.842 (0.37), 3.859 (0.19), 4.110 (0.17), 4.752 (0.23), 4.769 (0.31), 4.779 (0.32), 4.797 (1.07), 4.818 (0.90), 7.296 (0.38), 7.298 (0.37), 7.315 (0.26), 7.352 (0.24), 7.371 (0.37), 7.389 (0.16), 7.424 (0.48), 7.437 (0.53), 7.444 (0.49), 7.462 (0.48), 7.543 (0.36), 7.554 (0.36), 7.565 (0.37), 7.576 (0.35), 7.627 (0.75), 7.649 (1.15), 7.663 (0.42), 7.682 (0.36), 7.700 (0.44), 7.712 (1.22), 7.717 (0.74), 7.734 (0.75), 7.739 (0.51), 7.770 (0.21), 7.783 (0.37), 7.796 (0.19), 7.847 (0.58), 7.866 (0.53), 7.950 (0.55), 7.971 (0.45), 8.491 (0.57), 8.503 (0.55), 8.604 (0.95), 8.617 (0.44), 8.632 (0.79), 8.911 (0.46), 8.914 (0.47), 8.921 (0.46).

### Final Intermediate 36-3

### N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide

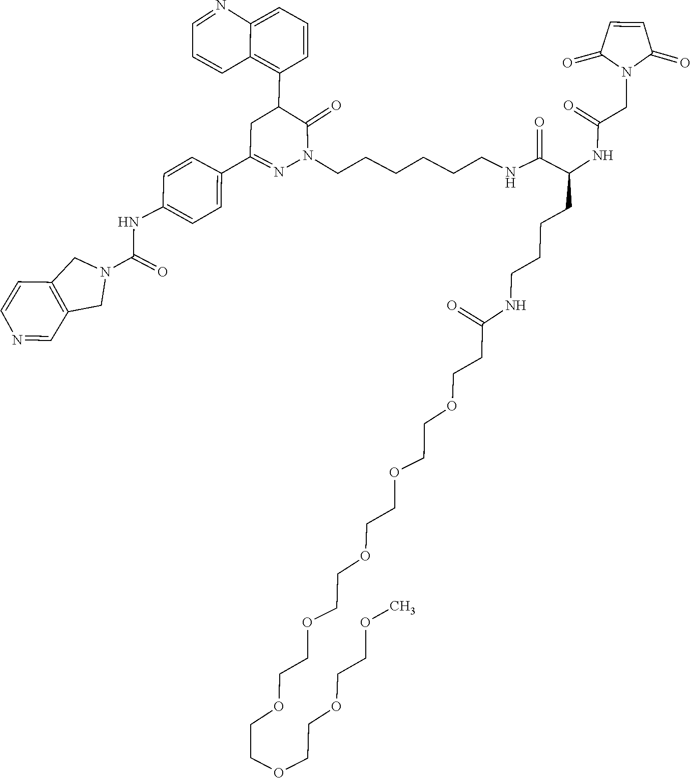 [SMILES: COCCOCCOCCOCCOCCOCCOCCOCCC(=O)NCCCC[C@H](NC(=O)CN1C(=O)C=CC1=O)C(=O)NCCCCCCN1N=C(c2ccc(NC(=O)N3Cc4ccncc4C3)cc2)CC(c2cccc3ncccc23)C1=O]

A mixture of N-{4-[6-oxo-1-(6-{[N6-(26-oxo-2,5,8,11,14,17,20,23-octaoxahexacosan-26-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide (41.2 mg, 36.1 μmol), 1-{2-[(2,5-dioxopyrrolidin-1-yl)oxy]-2-oxoethyl}-1H-pyrrole-2,5-dione (10.0 mg, 39.7 μmol) and N,N-diisopropylethylamine (13 μl, 72 μmol) in DMF (0.69 mL) was stirred at r.t. for 30 min under argon. Then the reaction was diluted with formic acid (2.7 μl, 72 μmol) in toluene (50 mL) and the mixture was concentrated under vacuum. The crude product was purified by column chromatography (SiO₂, dichloromethane/isopropyl alcohol, gradient containing 10% DMSO) to give 8.7 mg (95% purity, 18% yield) of the title compound.

LC-MS (Method 1): Rt=0.83 min; MS (ESIneg): m/z=1220 [M−H]⁻.

¹H-NMR (400 MHz, DMSO-d6) δ [ppm]: 1.13-1.28 (m, 3H), 1.28-1.50 (m, 8H), 1.55-1.75 (m, 3H), 2.27 (t, 2H), 2.94-3.09 (m, 4H), 3.23 (s, 3H), 3.26-3.30 (m, 1H), 3.36-3.44 (m, 4H), 3.44-3.52 (m, 26H), 3.56 (t, 2H), 3.77-3.89 (m, 2H), 4.08 (d, 2H), 4.11-4.19 (m, 1H), 4.74-4.85 (m, 5H), 7.07 (s, 2H), 7.42-7.48 (m, 2H), 7.56 (dd, 1H), 7.62-7.66 (m, 2H), 7.69-7.79 (m, 4H), 7.88-7.98 (m, 2H), 8.22-8.35 (m, 1H), 8.47-8.53 (m, 1H), 8.56-8.66 (m, 1H), 8.58-8.64 (m, 2H), 8.86-8.98 (m, 1H).

### Example 36A

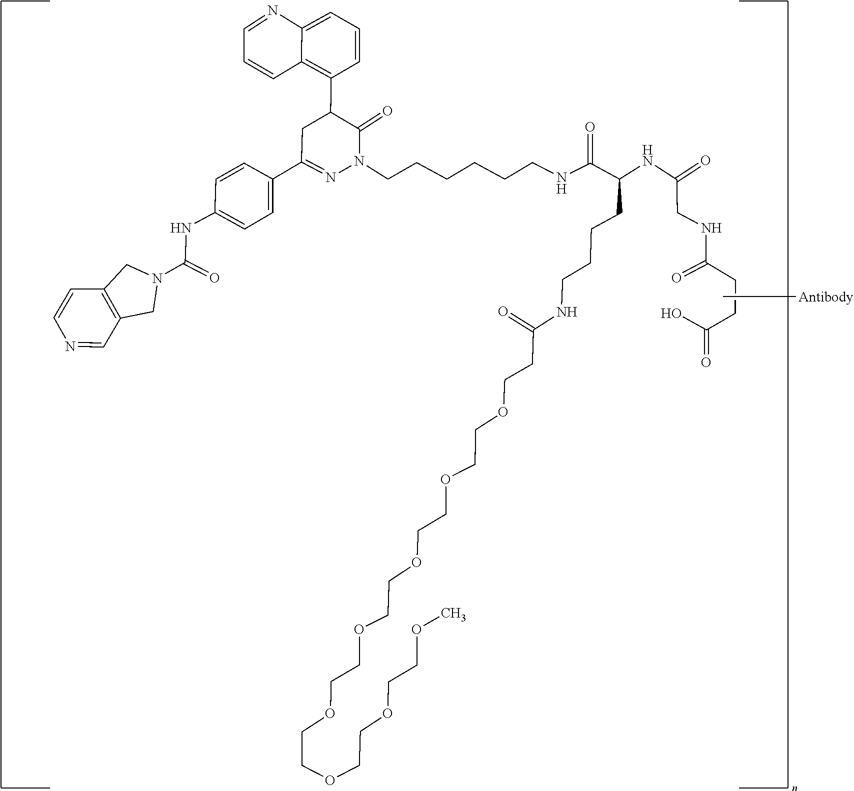

5 mg of anti-HER2 TPP-1015 (c=12.2 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide (340 μg, 95% purity, 0.27 μmol) according procedure 2 and the reaction was, after Sephadex purification, concentrated by ultracentrifugation and rediluted with PBS.

Protein concentration: 1.19 mg/mL

Drug/mAb ratio: 6.8 (UV)

### Example 36Ca

5 mg of anti-B7H3 TPP-8382 (c=15.1 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydro-pyridazin-3-yl]phenyl}-1,3-dihydro-2H-pyrrolo[3,4-c]pyridine-2-carboxamide (340 μg, 95% purity, 0.27 μmol) according procedure 2 and the reaction was, after Sephadex purification, concentrated by ultracentrifugation and rediluted with PBS.

Protein concentration: 1.58 mg/mL

Drug/mAb ratio: 4.3 (UV)

### Example 36Cb

5 mg of anti-B7H3 TPP-8382 (c=14.1 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide (690 μg, 95% purity, 0.53 μmol) according procedure 2 and the reaction was, after Sephadex purification, concentrated by ultracentrifugation and rediluted with PBS.

Protein concentration: 1.71 mg/mL

Drug/mAb ratio: 9.8 (UV)

### Example 36 Da

5 mg of anti-C4.4a TPP-509 (c=9.87 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydro-pyridazin-3-yl]phenyl}-1,3-dihydro-2H-pyrrolo[3,4-c]pyridine-2-carboxamide (340 μg, 95% purity, 0.27 μmol) according procedure 2 and the reaction was, after Sephadex purification, concentrated by ultracentrifugation and rediluted with PBS.

Protein concentration: 1.22 mg/mL

Drug/mAb ratio: 3.8 (UV)

### Example 36Db

### [(32S,39S)-39-carboxy-32-({6-[(5S)-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamoyl)-26,34,37-trioxo-2,5,8,11,14,17,20,23-octaoxa-27,33,36-triazanonatriacontan-39-yl]sulfanediyl

5 mg of anti-C4.4a TPP-509 (c=9.87 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydro-pyridazin-3-yl]phenyl}-1,3-dihydro-2H-pyrrolo[3,4-c]pyridine-2-carboxamide (690 μg, 95% purity, 0.53 μmol) according procedure 2 and the reaction was, after Sephadex purification, concentrated by ultracentrifugation and rediluted with PBS.

Protein concentration: 1.59 mg/mL

Drug/mAb ratio: 7.3 (UV)

### Example 36Ea

5 mg of anti-C4.4a TPP-668 (c=11.62 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydro-pyridazin-3-yl]phenyl}-1,3-dihydro-2H-pyrrolo[3,4-c]pyridine-2-carboxamide (340 μg, 95% purity, 0.27 μmol) according procedure 2 and the reaction was, after Sephadex purification, concentrated by ultracentrifugation and rediluted with PBS.

Protein concentration: 1.28 mg/mL

Drug/mAb ratio: 4.1 (UV)

### Example 36Eb

5 mg of anti-C4.4a TPP-668 (c=11.62 mg/mL) were coupled with Final Intermediate 36-3 N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydro-pyridazin-3-yl]phenyl}-1,3-dihydro-2H-pyrrolo[3,4-c]pyridine-2-carboxamide (690 μg, 95% purity, 0.53 μmol) according procedure 2 and the reaction was, after Sephadex purification, concentrated by ultracentrifugation and rediluted with PBS.

Protein concentration: 1.48 mg/mL

Drug/mAb ratio: 8.0 (UV)

### Intermediate 36-4

### S-{1-[(32S)-32-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamoyl)-26,34-dioxo-2,5,8,11,14,17,20,23-octaoxa-27,33-diazapentatriacontan-35-yl]-2,5-dioxopyrrolidin-3-yl}-N-{[2-(trimethylsilyl)ethoxy]carbonyl}-L-cysteine

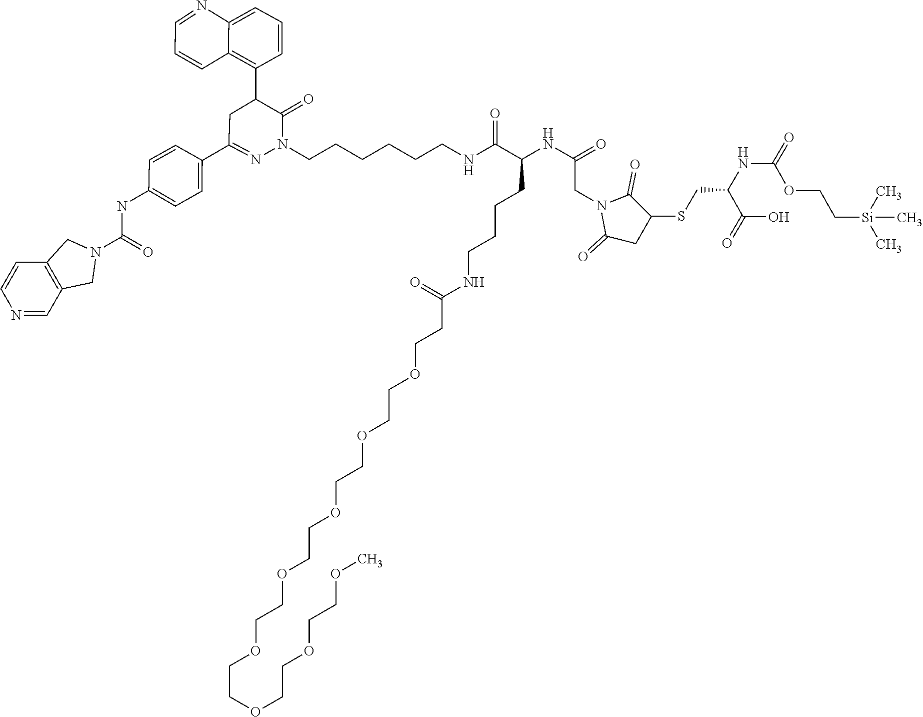

To a solution of N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Final Intermediate 36-3, 61.1 mg, 50.0 μmol) in DMF (3.8 mL) was added N-{[2-(trimethylsilyl)ethoxy]carbonyl}-L-cysteine (see Intermediate 30-2, 26.5 mg, 100 μmol) and the mixture was stirred for 1.5 h at r.t. Then toluene was added to the reaction solution and the mixture was concentrated under reduced pressure (azeotropic distillation with toluene was executed two times) to give the title compound as crude product which was directly used in the next step.

LC-MS (Method 1): Rₜ=0.81 min; MS (ESipos): m/z=744 [M+2H]²⁺; (ESIneg): m/z=1485 [M−H]⁻.

### Intermediate 36-5

### N-[(8R)-8,11-Dicarboxy-2,2-dimethyl-6,13-dioxo-5-oxa-10-thia-7-aza-2-silatridecan-13-yl]glycyl-N-{6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}-N⁶-(26-oxo-2,5,8,11,14,17,20,23-octaoxahexacosan-26-yl)-L-lysinamide

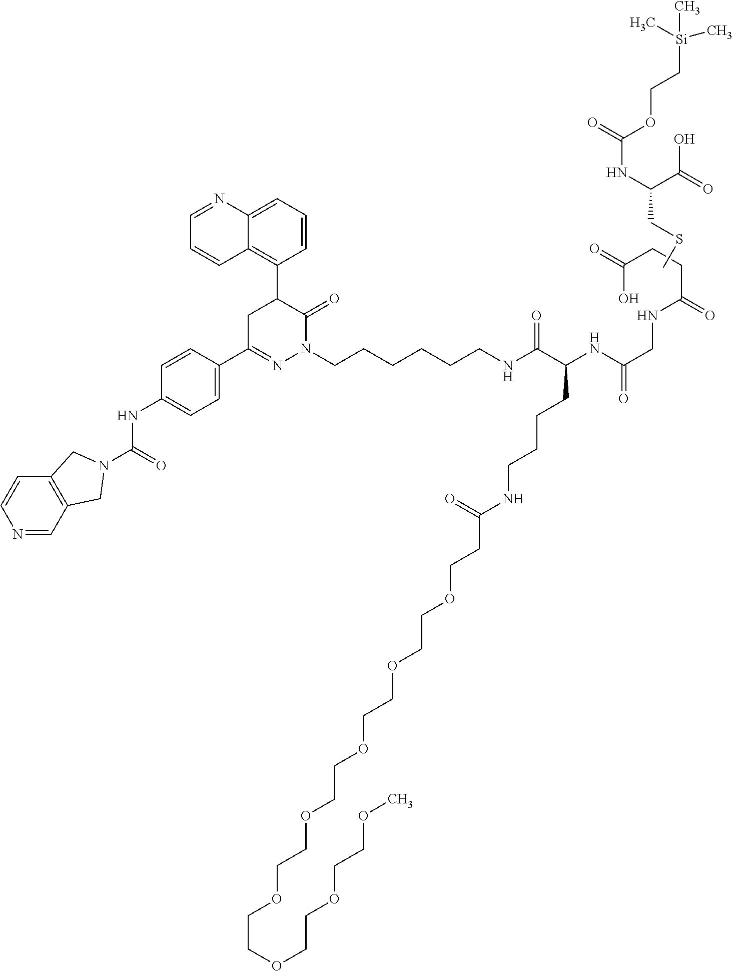

To a solution of S-{1-[(32S)-32-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamoyl)-26,34-dioxo-2,5,8,11,14,17,20,23-octaoxa-27,33-diazapentatriacontan-35-yl]-2,5-dioxo pyrrolidin-3-yl}-N-{[2-(trimethylsilyl)ethoxy]carbonyl}-L-cysteine (74.3 mg, 50.0 μmol) in a THF/water mixture 1:1 (3.0 mL) was added lithium hydroxide (7.8 mg, 325 μmol) in water (0.27 mL). After stirring for 1 h at r.t. the mixture was diluted with DMSO and the pH of the solution was adjusted to 5 by addition of formic acid (23 μl, 600 μmol). Purification by preparative HPLC yielded 55.5 mg (92% purity, 69% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm; Eluent A: water+0.1% formic acid; Eluent B: acetonitrile; gradient: 0-14 min 10-50% B, 14-22 min 50% B; rate 60 mL/min, temperature 25° C.

LC-MS (Method 1): Rₜ=0.95 min; MS (ESIpos): m/z=753 [M+2H]²⁺; (ESIneg): m/z=1503 [M−H]⁻.

¹H-NMR (400 MHz, DMSO-d6) δ [ppm]: −0.007 (1.90), 0.000 (2.09), 0.011 (0.74), 0.897 (0.17), 1.223 (0.46), 1.313 (0.41), 2.252 (0.17), 2.268 (0.34), 2.283 (0.19), 2.322 (1.01), 2.509 (6.26), 2.514 (4.17), 2.530 (16.00), 2.664 (1.03), 2.985 (0.19), 3.217 (2.64), 3.393 (0.48), 3.403 (0.60), 3.409 (0.48), 3.416 (0.65), 3.450 (0.74), 3.471 (2.66), 3.483 (6.31), 3.536 (0.26), 3.552 (0.46), 3.569 (0.24), 3.989 (0.17), 4.011 (0.22), 4.031 (0.17), 4.791 (0.36), 4.810 (0.36), 7.414 (0.19), 7.427 (0.22), 7.453 (0.19), 7.619 (0.26), 7.641 (0.41), 7.701 (0.46), 7.722 (0.29), 7.939 (0.24), 7.961 (0.19), 8.480 (0.22), 8.492 (0.22), 8.595 (0.36), 8.903 (0.19), 8.910 (0.17).

### Example 36M

### N-[2 or 3-{[(2R)-2-amino-2-carboxyethyl]sulfanyl}-3-carboxypropanoyl]glycyl-N-{6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}-N⁶-(26-oxo-2,5,8,11,14,17,20,23-octaoxahexacosan-26-yl)-L-lysinamide

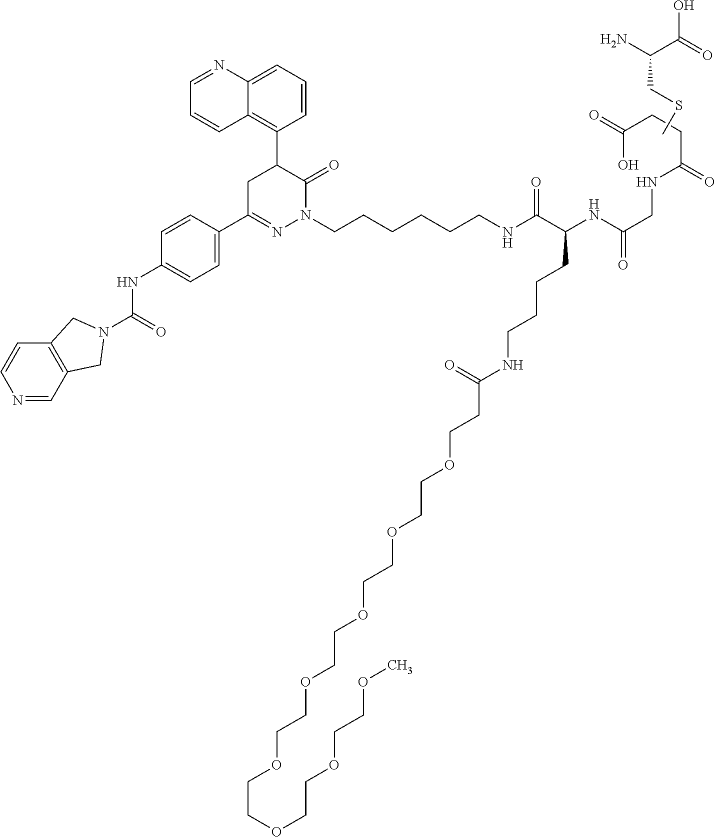

To N-[(8R)-8,11-dicarboxy-2,2-dimethyl-6,13-dioxo-5-oxa-10-thia-7-aza-2-silatridecan-13-yl]glycyl-N-{6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}-N⁶-(26-oxo-2,5,8,11,14,17,20,23-octaoxahexacosan-26-yl)-L-lysinamide (see Intermediate 36-5, 51.0 mg, 33.9 μmol) in 2,2,2-trifluoroethanol (4.7 mL) was added anhydrous zinc chloride (18.5 mg, 136 μmol) and the mixture was stirred for 4 h at 50° C. and 16 h at r.t. Anhydrous zinc chloride (9 mg, 68 μmol) was added and stirring was continued for 5 h at 50° C. Then the mixture was cooled down to r.t., ethylenedinitrilotetraacetic acid (10 mg) and formic acid in water (0.1%, 1 mL) were added and the mixture was concentrated under reduced pressure. The residue was diluted with dimethyl sulfoxide and it was purified by preparative HPLC to give 31.7 mg of the title compound (90% purity, 62% yield).

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-6 min 10-50% B, rate 150 mL/min, temperature 25° C.

LC-MS (Method 1): Rt=0.61 min; MS (ESIneg): m/z=789 [M−H]⁻

¹H-NMR (400 MHz, DMSO-d6) δ [ppm]: 1.232 (0.25), 1.323 (0.94), 1.401 (0.34), 1.696 (0.32), 2.266 (0.31), 2.281 (0.61), 2.296 (0.32), 2.322 (0.35), 2.326 (0.48), 2.331 (0.36), 2.522 (1.51), 2.664 (0.47), 2.669 (0.55), 2.983 (0.41), 3.010 (0.38), 3.027 (0.40), 3.175 (0.22), 3.226 (6.31), 3.402 (1.19), 3.412 (1.49), 3.418 (1.17), 3.425 (1.67), 3.455 (1.82), 3.461 (1.88), 3.481 (6.60), 3.493 (16.00), 3.506 (0.99), 3.547 (0.64), 3.563 (1.13), 3.580 (0.54), 3.602 (0.18), 3.621 (0.23), 3.640 (0.22), 3.700 (0.41), 3.825 (0.36), 4.141 (0.17), 4.155 (0.17), 4.752 (0.21), 4.770 (0.26), 4.805 (0.80), 4.823 (0.78), 7.422 (0.46), 7.434 (0.48), 7.445 (0.44), 7.464 (0.46), 7.544 (0.36), 7.555 (0.37), 7.566 (0.36), 7.576 (0.35), 7.634 (0.57), 7.656 (0.91), 7.702 (0.50), 7.709 (1.14), 7.722 (0.60), 7.732 (0.64), 7.740 (0.38), 7.836 (0.24), 7.928 (0.23), 7.950 (0.66), 7.971 (0.47), 8.489 (0.61), 8.502 (0.58), 8.603 (0.98), 8.617 (0.36), 8.677 (0.24), 8.696 (0.33), 8.911 (0.47), 8.914 (0.47), 8.921 (0.46).

### Intermediate 37-1

### Tert-Butyl (6-{5-[6-{4-[(1,3-dihydro-2H-pyrrolo[3,4-c]pyridine-2-carbonyl)amino]phenyl}-3-oxo-2-(2,2,2-trifluoroethyl)-2,3-dihydropyridazin-4-yl]-1H-indazol-1-yl}hexyl)carbamate

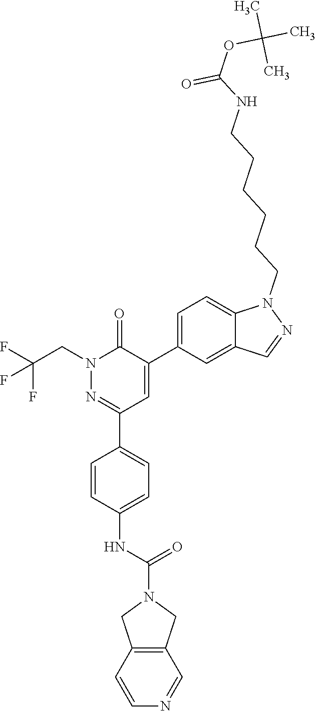 [SMILES: CC(C)(C)OC(=O)NCCCCCCn1ncc2cc(-c3cc(-c4ccc(NC(=O)N5Cc6ccncc6C5)cc4)nn(CC(F)(F)F)c3=O)ccc21]

To a suspension of N-{4-[5-(1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide hydrogen chloride (see Example 22, raw product before HPLC purification was used, 100 mg, 176 μmol) in DMF (1.9 mL) was added at 0° C. sodium hydride (14.8 mg, 60% in mineral oil, 370 μmol) under an argon atmosphere. The mixture was stirred for 30 min at that temperature. Then tetra-n-butylammonium iodide (6.50 mg, 17.6 μmol) and tert-butyl (6-bromohexyl)carbamate (41 μl, 180 μmol) were added. After stirring for additional 2 h at 0° C. the mixture was was diluted with aqueous ammonium chloride solution, and directly purified by preparative HPLC to yield 24.3 mg (80% purity, 15% yield) of the title compound and 17.4 mg (95% purity, 13% yield) of tert-butyl(6-{5-[6-{4-[(1,3-dihydro-2H-pyrrolo[3,4-c]pyridin-2-ylcarbonyl)amino]phenyl}-3-oxo-2-(2,2,2-trifluoroethyl)-2,3-dihydropyridazin-4-yl]-2H-indazol-2-yl}hexyl)carbamate as a second product.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-6 min 40-80% B, 6-8 min 80-100% B, rate 150 mL/min, temperature 25° C.

LC-MS (Method 2): Rₜ=1.31 min; MS (ESipos): m/z=731 [M+H]⁺.

¹H-NMR (400 MHz, DMSO-d6) δ [ppm]: 1.231 (1.07), 1.256 (0.81), 1.275 (0.72), 1.324 (0.69), 1.353 (16.00), 1.366 (6.83), 1.793 (0.32), 1.812 (0.41), 1.830 (0.54), 1.848 (0.38), 2.326 (0.92), 2.331 (0.75), 2.664 (0.68), 2.668 (0.95), 2.673 (0.78), 2.848 (0.64), 2.863 (0.78), 2.879 (0.64), 2.893 (0.43), 3.500 (0.38), 3.516 (0.80), 3.533 (0.40), 4.431 (0.46), 4.447 (0.92), 4.464 (0.48), 4.834 (1.07), 4.852 (1.11), 5.114 (0.55), 5.137 (0.54), 6.752 (0.38), 7.443 (0.54), 7.456 (0.57), 7.742 (1.07), 7.764 (1.43), 7.791 (0.66), 7.939 (1.24), 7.948 (0.84), 7.951 (0.83), 7.961 (1.15), 7.970 (0.60), 7.974 (0.60), 8.194 (1.49), 8.240 (1.41), 8.474 (0.98), 8.505 (0.69), 8.517 (0.68), 8.624 (1.06), 8.682 (0.92).

### Second Product: Tert-Butyl(6-{5-[6-{4-[(1,3-dihydro-2H-pyrrolo[3,4-c]pyridin-2-ylcarbonyl)amino] phenyl}-3-oxo-2-(2,2,2-trifluoroethyl)-2,3-dihydropyridazin-4-yl]-2H-indazol-2-yl}hexyl) Carbamate

LC-MS (Method 2): Rₜ=1.26 min; MS (ESipos): m/z=731 [M+H]⁺.

¹H-NMR (400 MHz, DMSO-d6) δ [ppm]: 1.200 (0.21), 1.231 (0.45), 1.254 (0.37), 1.272 (0.30), 1.294 (0.27), 1.309 (0.28), 1.323 (0.34), 1.341 (0.43), 1.359 (16.00), 1.905 (0.26), 1.922 (0.37), 1.940 (0.28), 2.518 (3.00), 2.523 (2.43), 2.539 (0.49), 2.848 (0.20), 2.865 (0.45), 2.880 (0.47), 2.897 (0.18), 4.429 (0.33), 4.446 (0.70), 4.463 (0.35), 4.833 (0.80), 4.851 (0.83), 5.109 (0.39), 5.132 (0.39), 6.766 (0.26), 7.442 (0.45), 7.454 (0.48), 7.671 (0.45), 7.694 (0.66), 7.740 (0.99), 7.763 (1.53), 7.768 (0.78), 7.786 (0.39), 7.791 (0.40), 7.934 (1.18), 7.957 (0.95), 8.207 (1.37), 8.483 (0.76), 8.485 (0.75), 8.487 (0.65), 8.504 (0.71), 8.517 (0.66), 8.548 (1.06), 8.623 (0.91), 8.681 (0.80).

### Intermediate 37-2

### N-(4-{5-[1-(6-Aminohexyl)-1H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide

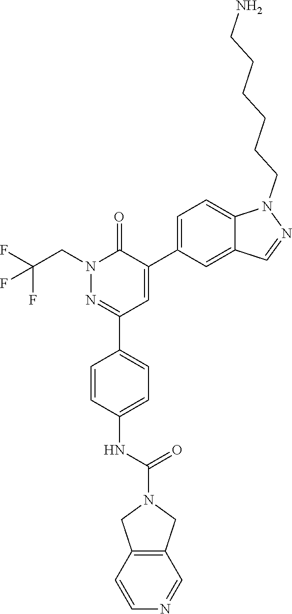 [SMILES: NCCCCCCn1ncc2cc(-c3cc(-c4ccc(NC(=O)N5Cc6ccncc6C5)cc4)nn(CC(F)(F)F)c3=O)ccc21]

A mixture of tert-butyl (6-{5-[6-{4-[(1,3-dihydro-2H-pyrrolo[3,4-c]pyridine-2-carbonyl) amino]phenyl}-3-oxo-2-(2,2,2-trifluoroethyl)-2,3-dihydropyridazin-4-yl]-1H-indazol-1-yl}hexyl) carbamate (23.0 mg, 31.5 μmol) and trifluoroacetic acid (150 μl, 1.9 mmol) in dichloromethane (1.4 mL) was stirred at r.t. for 30 min. Then the mixture was concentrated under reduced pressure and the raw product was purified by preparative HPLC to give 16.2 mg (95% purity, 78% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm. Eluent A: water+0.1% formic acid; Eluent B: acetonitrile; gradient: 0-14 min 15-55% B, 14-17 min 55-100% B, rate 60 mL/min, temperature 25° C.

LC-MS (Method 1): Rₜ=0.84 min; MS (ESIneg): m/z=629 [M−H]⁻.

¹H-NMR (400 MHz, DMSO-d6) δ [ppm]: 1.17-1.25 (m, 2H), 1.28-1.35 (m, 2H), 1.37-1.47 (m, 2H), 1.75-1.91 (m, 2H), 2.63 (t, 2H), 4.46 (t, 2H), 4.84 (br d, 4H), 5.13 (q, 2H), 7.45 (d, 1H), 7.76 (d, 2H), 7.79 (d, 1H), 7.89-8.01 (m, 3H), 8.20 (s, 1H), 8.24 (s, 1H), 8.47 (d, 1H), 8.51 (d, 1H), 8.62 (s, 1H), 8.70 (s, 1H).

### Final Intermediate 37-3

### N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide

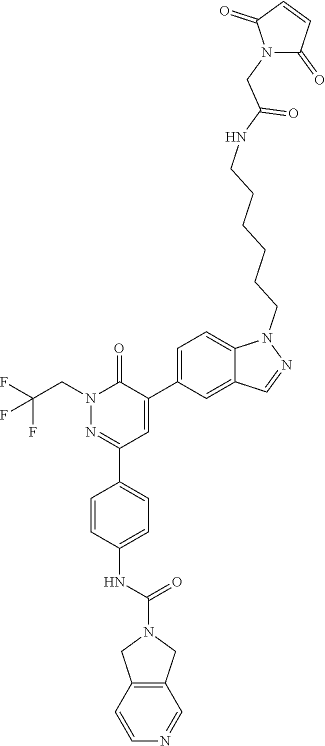 [SMILES: O=C(CN1C(=O)C=CC1=O)NCCCCCCn1ncc2cc(-c3cc(-c4ccc(NC(=O)N5Cc6ccncc6C5)cc4)nn(CC(F)(F)F)c3=O)ccc21]

A mixture of N-(4-{5-[1-(6-aminohexyl)-1H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide (15.1 mg, 23.9 μmol), 1-{2-[(2,5-dioxopyrrolidin-1-yl)oxy]-2-oxoethyl}-1H-pyrrole-2,5-dione (6.04 mg, 23.9 μmol) and N,N-diisopropylethylamine (8.3 μl, 48 μmol) in DMF (0.46 mL) was stirred at r.t. for 30 min under argon. Then formic acid (1.8 μl, 48 μmol) was added and the mixture was concentrated under vacuum. The crude product was purified by preparative HPLC to give 5.35 mg (75% purity, 22% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG-10 μm 12 nm 150×20 mm; Eluent A: water+0.1% formic acid; Eluent B: acetonitrile; gradient: 0-14 min 15-55% B, 14-17 min 55-100% B; rate 60 mL/min, temperature 25° C.

LC-MS (Method 1): Rₜ=1.00 min; MS (ESipos): m/z=768 [M+H]⁺.

¹H-NMR (400 MHz, DMSO-d6) δ [ppm]: 1.232 (2.04), 1.265 (1.41), 1.308 (1.21), 1.325 (1.45), 1.342 (1.45), 1.818 (1.21), 1.836 (1.58), 1.855 (1.12), 2.978 (0.79), 2.995 (1.95), 3.010 (2.04), 3.025 (0.91), 3.977 (8.10), 4.436 (1.54), 4.453 (2.95), 4.470 (1.41), 4.833 (3.66), 4.851 (3.53), 5.092 (0.71), 5.114 (1.75), 5.137 (1.66), 7.081 (16.00), 7.087 (1.00), 7.442 (1.91), 7.455 (1.91), 7.741 (4.11), 7.763 (4.82), 7.772 (2.20), 7.795 (2.20), 7.939 (4.95), 7.948 (2.91), 7.951 (2.74), 7.962 (4.11), 7.970 (1.91), 7.974 (1.70), 8.060 (0.66), 8.074 (1.21), 8.087 (0.66), 8.196 (5.03), 8.241 (5.36), 8.471 (3.16), 8.473 (3.24), 8.505 (2.12), 8.517 (1.95), 8.623 (3.16), 8.683 (3.32).

### Example 37A

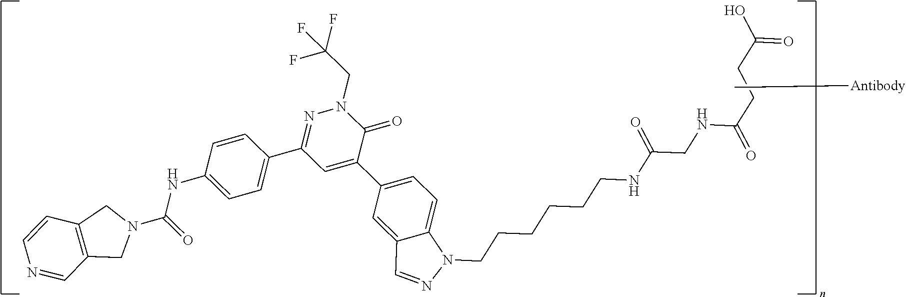

5 mg of anti-HER2 TPP-1015 (c=12.2 mg/mL) were coupled with N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 37-3, 230 μg, 90% purity, 0.27 μmol) according procedure 2 and the reaction was, after Sephadex purification, concentrated by ultracentrifugation and rediluted with PBS.

Protein concentration: 0.99 mg/mL

Drug/mAb ratio: 3.1 (LC-MS)

### Example 37C

5 mg of anti-B7H3 TPP-8382 (c=14.08 mg/mL) were coupled with N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 37-3, 230 μg, 90% purity, 0.27 μmol) according procedure 2 and the reaction was, after Sephadex purification, concentrated by ultracentrifugation and rediluted with PBS.

Protein concentration: 2.98 mg/mL

Drug/mAb ratio: 3.7 (UV)

### Example 37D

5 mg of anti-C4.4a TPP-509 (c=9.87 mg/mL) were coupled with N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 37-3, 230 μg, 90% purity, 0.27 μmol) according procedure 2 and the reaction was, after Sephadex purification, concentrated by ultracentrifugation and rediluted with PBS.

Protein concentration: 0.23 mg/mL

Drug/mAb ratio: 1.0 (LC-MS)

### Example 37E

5 mg of anti-C4.4a TPP-668 (c=11.62 mg/mL) were coupled with N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 37-3, 230 μg, 90% purity, 0.27 μmol) according procedure 2 and the reaction was, after Sephadex purification, concentrated by ultracentrifugation and rediluted with PBS.

Protein concentration: 1.48 mg/mL

Drug/mAb ratio: 1.7 (LC-MS)

### Intermediate 38-1

### N-(4-{5-[2-(6-aminohexyl)-2H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide

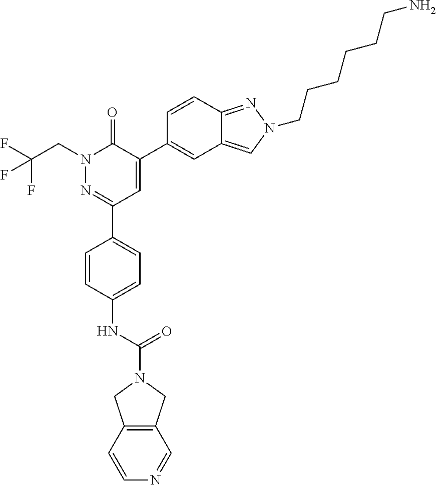 [SMILES: NCCCCCCn1cc2cc(-c3cc(-c4ccc(NC(=O)N5Cc6ccncc6C5)cc4)nn(CC(F)(F)F)c3=O)ccc2n1]

A mixture of tert-butyl (6-{5-[6-{4-[(1,3-dihydro-2H-pyrrolo[3,4-c]pyridine-2-carbonyl)amino]phenyl}-3-oxo-2-(2,2,2-trifluoroethyl)-2,3-dihydropyridazin-4-yl]-2H-indazol-2-yl}hexyl) carbamate (see Intermediate 37-1 second product, 16.0 mg, 21.9 μmol) and trifluoroacetic acid (100 μL, 1.3 mmol) in dichloromethane (1.0 mL) was stirred at r.t. for 30 min. Then the mixture was concentrated under reduced pressure and the raw product was purified by preparative HPLC to give 12.8 mg (95% purity, 88% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm. Eluent A: water+0.1% formic acid; Eluent B: acetonitrile; gradient: 0-14 min 15-55% B, 14-17 min 55-100% B, rate 60 mL/min, temperature 25° C.

LC-MS (Method 1): Rₜ=0.79 min; MS (ESIneg): m/z=629 [M−H]⁻.

¹H-NMR (400 MHz, DMSO-d6) δ [ppm]: 1.14-1.28 (m, 2H), 1.28-1.38 (m, 2H), 1.39-1.49 (m, 2H), 1.85-2.04 (m, 2H), 2.66 (t, 2H), 4.46 (t, 2H), 4.84 (br d, 4H), 5.12 (q, 1H), 5.16 (br s, 1H), 7.45 (d, 1H), 7.69 (d, 1H), 7.72-7.81 (m, 3H), 7.94 (d, 2H), 8.21 (s, 1H), 8.39-8.46 (m, 1H), 8.47-8.53 (m, 2H), 8.55 (s, 1H), 8.62 (s, 1H), 8.70 (s, 1H).

### Final Intermediate 38-2

### N-{4-[5-(2-{6-[2-(2,5-dioxo-2,5-dihydro-1H-pyrrol-1-yl)acetamido]hexyl}-2H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide

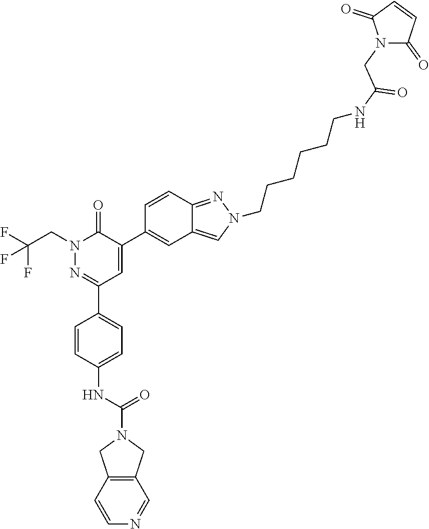 [SMILES: O=C(CN1C(=O)C=CC1=O)NCCCCCCn1cc2cc(-c3cc(-c4ccc(NC(=O)N5Cc6ccncc6C5)cc4)nn(CC(F)(F)F)c3=O)ccc2n1]

A mixture of N-(4-{5-[2-(6-aminohexyl)-2H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide (11.8 mg, 18.7 μmol), 1-{2-[(2,5-dioxopyrrolidin-1-yl)oxy]-2-oxoethyl}-1H-pyrrole-2,5-dione (4.72 mg, 18.7 μmol) and N,N-diisopropylethylamine (6.5 μl, 37 μmol) in DMF (0.36 mL) was stirred at r.t. for 30 min. Then formic acid (1.4 μl, 37 μmol) was added and the mixture was concentrated under reduced pressure. The crude product was purified by preparative HPLC to give 2.97 mg (75% purity, 16% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG-10 μm 12 nm 150×20 mm; Eluent A: water+0.1% formic acid; Eluent B: acetonitrile; gradient: 0-14 min 15-55% B, 14-17 min 55-100% B; rate 60 mL/min, temperature 25° C.

LC-MS (Method 1): Rₜ=0.95 min: MS (ESIpos): m/z=768 [M+H]⁺.

¹H-NMR (400 MHz, DMSO-d6) δ [ppm]: 1.231 (1.81), 1.256 (1.21), 1.270 (1.12), 1.292 (1.12), 1.328 (0.74), 1.352 (1.21), 1.366 (1.26), 1.910 (1.02), 1.928 (1.40), 1.946 (0.98), 2.995 (0.70), 3.012 (1.67), 3.026 (1.72), 3.042 (0.70), 3.984 (7.67), 4.433 (1.30), 4.451 (2.56), 4.468 (1.26), 4.833 (3.07), 4.851 (2.98), 5.086 (0.60), 5.109 (1.49), 5.131 (1.40), 5.154 (0.51), 7.087 (16.00), 7.442 (1.63), 7.456 (1.63), 7.674 (1.67), 7.697 (2.42), 7.739 (3.72), 7.762 (6.28), 7.766 (2.79), 7.785 (1.35), 7.789 (1.40), 7.935 (4.37), 7.957 (3.40), 7.972 (0.51), 8.072 (0.56), 8.086 (1.12), 8.100 (0.56), 8.208 (4.98), 8.482 (2.84), 8.505 (1.81), 8.517 (1.67), 8.552 (3.81), 8.623 (2.65), 8.682 (2.88).

### Example 38A

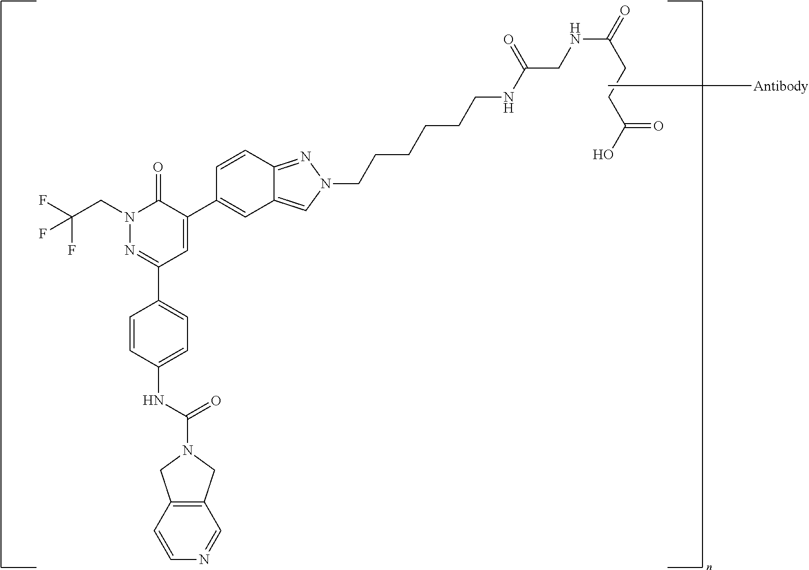

5 mg of anti-HER2 TPP-1015 (c=12.2 mg/mL) were coupled with N-{4-[5-(2-{6-[2-(2,5-dioxo-2,5-dihydro-1H-pyrrol-1-yl)acetamido]hexyl}-2H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 38-2, 230 μg, 90% purity, 0.27 μmol) according procedure 2 and the reaction was, after Sephadex purification, concentrated by ultracentrifugation and rediluted with PBS.

Protein concentration: 2.73 mg/mL

Drug/mAb ratio: 4.0 (LC-MS)

### Example 38B

5 mg of anti-CXCR5 TPP-9574 (c=10.65 mg/mL) were coupled with N-{4-[5-(2-{6-[2-(2,5-dioxo-2,5-dihydro-1H-pyrrol-1-yl)acetamido]hexyl}-2H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (Final intermediate 38-2, 230 μg, 90% purity, 0.27 μmol) according to procedure 2 and the reaction was, after Sephadex purification, concentrated by ultracentrifugation and rediluted with PBS.

Protein concentration: 1.88 mg/mL

Drug/mAb ratio: 3.1 (LC-MS)

### Example 38C

5 mg of anti-B7H3 TPP-8382 (c=14.08 mg/mL) were coupled with N-{4-[5-(2-{6-[2-(2,5-dioxo-2,5-dihydro-1H-pyrrol-1-yl)acetamido]hexyl}-2H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 38-2, 230 μg, 90% purity, 0.27 μmol) according procedure 2 and the reaction was, after Sephadex purification, concentrated by ultracentrifugation and rediluted with PBS.

Protein concentration: 2.37 mg/mL

Drug/mAb ratio: 3.7 (UV)

### Intermediate 39-1

### Tert-Butyl (6-{4-[6-{4-[(1,3-dihydro-2H-pyrrolo[3,4-c]pyridin-2-ylcarbonyl)amino]phenyl}-3-oxo-2-(2,2,2-trifluoroethyl)-2,3-dihydropyridazin-4-yl]-1H-indazol-1-yl}hexyl)carbamate

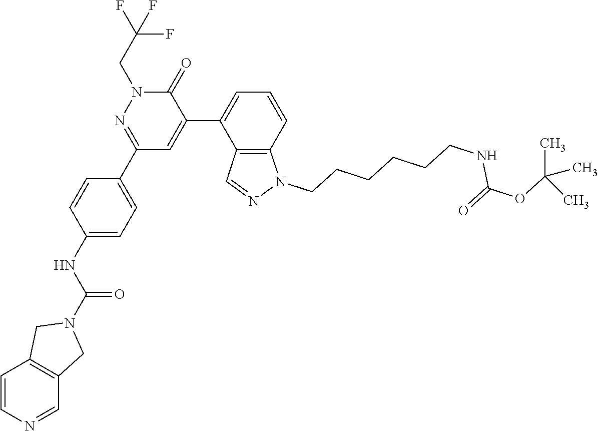 [SMILES: CC(C)(C)OC(=O)NCCCCCCn1ncc2c(-c3cc(-c4ccc(NC(=O)N5Cc6ccncc6C5)cc4)nn(CC(F)(F)F)c3=O)cccc21]

To a suspension of N-{4-[5-(1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide hydrogen chloride (see Example 23, dried raw product before aqueous sodium bicarbonate treatment was used, 96.0 mg, 169 μmol) in DMF (1.8 mL) was added at 0° C. sodium hydride (14.2 mg, 60% in mineral oil, 355 μmol) under an argon atmosphere. The mixture stirred for 30 min at that temperature. Then tetra-n-butylammonium iodide (6.24 mg, 16.9 μmol) and tert-butyl (6-bromohexyl)carbamate (40 μl, 170 μmol) was added. After stirring for additional 3 h at r.t. the mixture was was diluted with aqueous ammonium chloride solution, and directly purified by preparative HPLC to yield 50.8 mg (75% purity, 31% yield) of the title compound and 33.4 mg (95% purity, 26% yield) of tert-butyl (6-{4-[6-{4-[(1,3-dihydro-2H-pyrrolo[3,4-c]pyridine-2-carbonyl)amino]phenyl}-3-oxo-2-(2,2,2-trifluoroethyl)-2,3-dihydropyridazin-4-yl]-2H-indazol-2-yl}hexyl)carbamate as a second product.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% ammonia; Eluent B: acetonitrile; gradient: 0-6 min 40-80% B, 6-8 min 80-100% B, rate 150 mL/min, temperature 25° C.

LC-MS (Method 2): Rₜ=1.31 min; MS (ESIpos): m/z=731 [M+H]⁺

¹H-NMR (400 MHz, DMSO-d6) δ [ppm]: 1.259 (1.38), 1.356 (16.00), 1.366 (6.80), 1.776 (0.33), 1.794 (0.36), 1.811 (0.47), 1.828 (0.59), 2.074 (1.13), 2.860 (0.84), 2.876 (0.95), 2.889 (0.74), 3.500 (0.37), 3.517 (0.71), 3.534 (0.35), 4.432 (0.53), 4.449 (1.01), 4.466 (0.50), 4.829 (1.39), 4.848 (1.38), 5.133 (0.65), 5.156 (0.63), 6.760 (0.48), 6.773 (0.35), 7.440 (0.68), 7.452 (0.70), 7.501 (1.33), 7.512 (1.39), 7.733 (1.17), 7.754 (1.44), 7.809 (0.43), 7.820 (0.59), 7.832 (0.37), 7.908 (1.40), 7.930 (1.11), 8.037 (1.64), 8.231 (1.47), 8.503 (0.79), 8.515 (0.77), 8.621 (1.30), 8.684 (1.12).

### Second Product: Tert-Butyl (6-{4-[6-{4-[(1,3-dihydro-2H-pyrrolo[3,4-c]pyridine-2-carbonyl)amino]phenyl}-3-oxo-2-(2,2,2-trifluoroethyl)-2,3-dihydropyridazin-4-yl]-2H-indazol-2-yl}hexyl)carbamate

LC-MS (Method 2): Rₜ=1.26 min, MS (ESipos): m/z=731 [M+H]⁺.

¹H-NMR (400 MHz, DMSO-d6) δ [ppm]: 1.231 (1.81), 1.256 (1.21), 1.270 (1.12), 1.292 (1.12), 1.328 (0.74), 1.352 (1.21), 1.366 (1.26), 1.910 (1.02), 1.928 (1.40), 1.946 (0.98), 2.995 (0.70), 3.012 (1.67), 3.026 (1.72), 3.042 (0.70), 3.984 (7.67), 4.433 (1.30), 4.451 (2.56), 4.468 (1.26), 4.833 (3.07), 4.851 (2.98), 5.086 (0.60), 5.109 (1.49), 5.131 (1.40), 5.154 (0.51), 7.087 (16.00), 7.442 (1.63), 7.456 (1.63), 7.674 (1.67), 7.697 (2.42), 7.739 (3.72), 7.762 (6.28), 7.766 (2.79), 7.785 (1.35), 7.789 (1.40), 7.935 (4.37), 7.957 (3.40), 7.972 (0.51), 8.072 (0.56), 8.086 (1.12), 8.100 (0.56), 8.208 (4.98), 8.482 (2.84), 8.505 (1.81), 8.517 (1.67), 8.552 (3.81), 8.623 (2.65), 8.682 (2.88).

### Intermediate 39-2

### N-(4-{5-[1-(6-Aminohexyl)-1H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide

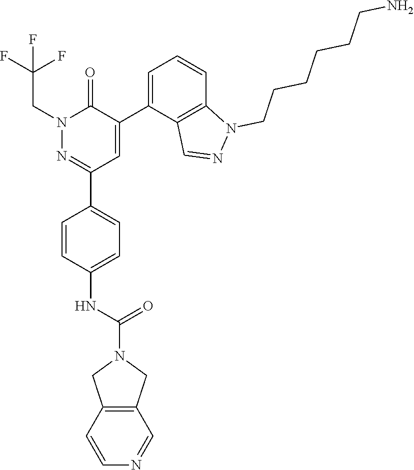 [SMILES: NCCCCCCn1ncc2c(-c3cc(-c4ccc(NC(=O)N5Cc6ccncc6C5)cc4)nn(CC(F)(F)F)c3=O)cccc21]

A mixture of tert-butyl (6-{4-[6-{4-[(1,3-dihydro-2H-pyrrolo[3,4-c]pyridine-2-carbonyl) amino]phenyl}-3-oxo-2-(2,2,2-trifluoroethyl)-2,3-dihydropyridazin-4-yl]-1H-indazol-1-yl}hexyl) carbamate (51.8 mg, 70.9 μmol) and trifluoroacetic acid (330 μL, 4.3 mmol) in dichloromethane (3.2 mL) was stirred at r.t. for 30 min. Then the mixture was concentrated under reduced pressure and the raw product was purified by preparative HPLC to give 26.4 mg (95% purity, 56% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% formic acid; Eluent B: acetonitrile; gradient: 0-6 min 15-55% B, 6-8 min 55-100% B, rate 150 mL/min, temperature 25° C.

LC-MS (Method 1): Rₜ=0.80 min; MS (ESIneg): m/z=629 [M−H]⁻.

¹H-NMR (400 MHz, DMSO-d6) δ [ppm]: 1.256 (4.15), 1.271 (3.83), 1.295 (3.03), 1.313 (3.56), 1.332 (3.88), 1.352 (2.92), 1.369 (1.22), 1.432 (1.54), 1.450 (3.61), 1.468 (4.41), 1.487 (2.71), 1.826 (3.56), 1.844 (4.68), 1.862 (3.35), 2.680 (4.47), 2.699 (6.33), 2.717 (3.88), 3.352 (3.35), 4.448 (4.41), 4.465 (8.45), 4.482 (4.04), 4.828 (10.26), 4.846 (10.37), 5.111 (1.97), 5.134 (5.16), 5.157 (5.00), 5.179 (1.70), 7.438 (5.53), 7.452 (5.69), 7.495 (2.45), 7.505 (15.79), 7.513 (8.45), 7.520 (8.35), 7.537 (1.65), 7.735 (10.95), 7.757 (12.97), 7.818 (3.77), 7.827 (3.40), 7.831 (3.46), 7.840 (3.24), 7.906 (13.45), 7.929 (10.26), 8.036 (14.09), 8.038 (14.41), 8.226 (16.00), 8.384 (2.18), 8.503 (6.86), 8.516 (6.54), 8.621 (10.26), 8.704 (8.98).

### Final Intermediate 39-3

### N-{4-[5-(1-{6-[2-(2,5-dioxo-2,5-dihydro-1H-pyrrol-1-yl)acetamido]hexyl}-1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide

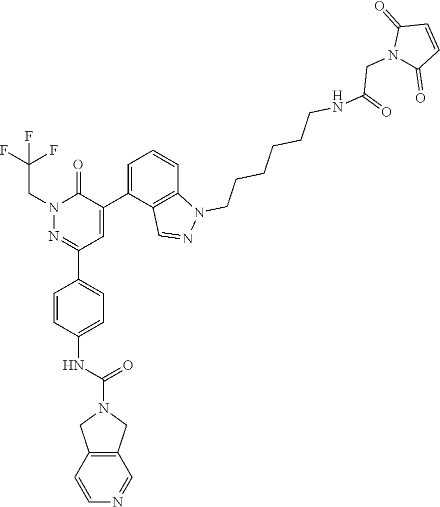 [SMILES: O=C(CN1C(=O)C=CC1=O)NCCCCCCn1ncc2c(-c3cc(-c4ccc(NC(=O)N5Cc6ccncc6C5)cc4)nn(CC(F)(F)F)c3=O)cccc21]

A mixture of N-(4-{5-[1-(6-aminohexyl)-1H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide (26.4 mg, 41.9 μmol), 1-{2-[(2,5-dioxopyrrolidin-1-yl)oxy]-2-oxoethyl}-1H-pyrrole-2,5-dione (10.6 mg, 41.9 μmol) and N,N-diisopropylethylamine (15 μL, 84 μmol) in DMF (0.81 mL) was stirred at r.t. for 30 min under argon. Then formic acid (3.2 μL, 84 μmol) was added and the mixture was concentrated under vacuum. The crude product was purified by preparative HPLC to give 5.70 mg (70% purity, 12% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm; Eluent A: water+0.1% formic acid; Eluent B: acetonitrile; gradient: 0-14 min 15-55% B, 14-17 min 55-100% B; rate 60 mL/min, temperature 25° C.

LC-MS (Method 1): Rₜ=1.01 min; MS (ESIpos): m/z=768 [M+H]⁺.

¹H-NMR (400 MHz, DMSO-d6) δ [ppm]: 1.265 (3.47), 1.352 (2.12), 1.833 (2.03), 2.987 (1.10), 3.003 (2.54), 3.018 (2.54), 3.917 (1.19), 3.979 (8.72), 4.437 (1.95), 4.454 (3.47), 4.471 (1.69), 4.829 (4.06), 4.846 (3.81), 5.133 (2.12), 5.155 (2.03), 7.084 (14.81), 7.440 (2.12), 7.452 (2.12), 7.502 (6.10), 7.511 (3.47), 7.516 (3.56), 7.731 (4.40), 7.754 (4.91), 7.812 (1.61), 7.823 (1.52), 7.835 (1.27), 7.909 (5.08), 7.930 (3.98), 8.038 (5.33), 8.080 (1.44), 8.233 (5.67), 8.502 (1.95), 8.515 (1.78), 8.621 (3.13), 8.684 (3.39).

### Example 39A

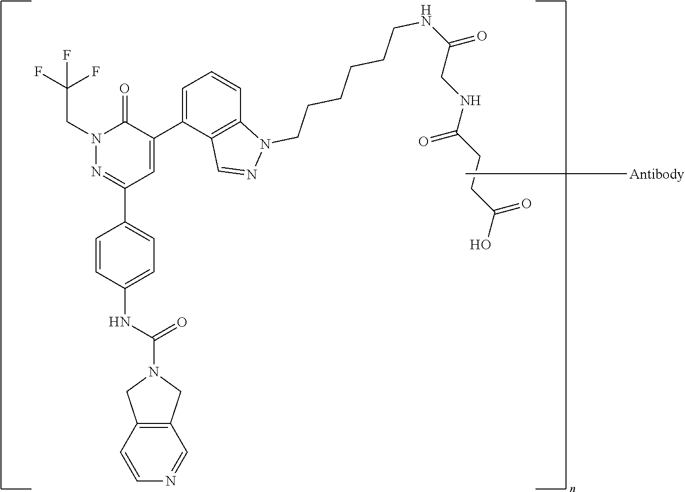

5 mg of anti-HER2 TPP-1015 (c=12.2 mg/mL) were coupled with N-{4-[5-(1-{6-[2-(2,5-dioxo-2,5-dihydro-1H-pyrrol-1-yl)acetamido]hexyl}-1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 39-3, 290 μg, 70% purity, 0.27 μmol) according procedure 2 and the reaction was, after Sephadex purification, concentrated by ultracentrifugation and rediluted with PBS.

Protein concentration: 1.66 mg/mL

Drug/mAb ratio: 0.6 (LC-MS)

### Example 39C

5 mg of anti-B7H3 TPP-8382 (c=14.08 mg/mL) were coupled with N-{4-[5-(1-{6-[2-(2,5-dioxo-2,5-dihydro-1H-pyrrol-1-yl)acetamido]hexyl}-1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 39-3, 290 μg, 70% purity, 0.27 μmol) according procedure 2 and the reaction was, after Sephadex purification, concentrated by ultracentrifugation and rediluted with PBS.

Protein concentration: 1.55 mg/mL

Drug/mAb ratio: 0.5 (UV)

### Intermediate 40-1

### N-(4-{5-[2-(6-Aminohexyl)-2H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide

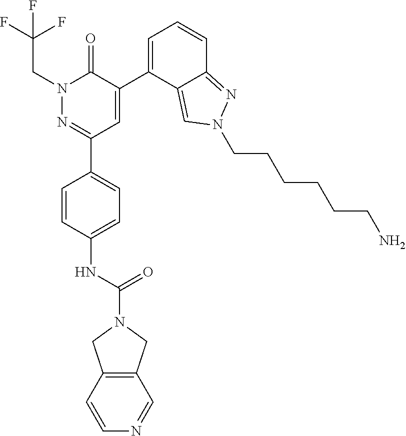 [SMILES: NCCCCCCn1cc2c(-c3cc(-c4ccc(NC(=O)N5Cc6ccncc6C5)cc4)nn(CC(F)(F)F)c3=O)cccc2n1]

A mixture of tert-butyl (6-{4-[6-{4-[(1,3-dihydro-2H-pyrrolo[3,4-c]pyridin-2-ylcarbonyl) amino]phenyl}-3-oxo-2-(2,2,2-trifluoroethyl)-2,3-dihydropyridazin-4-yl]-2H-indazol-2-yl}hexyl) carbamate (intermediate 39-1 second product 33.4 mg, 45.7 μmol) and trifluoroacetic acid (210 μL, 2.7 mmol) in dichloromethane (2.1 mL) was stirred at r.t. for 30 min. Then the mixture was concentrated under reduced pressure and the raw product was purified by preparative HPLC to give 11.3 mg (95% purity, 37% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 125×30 mm. Eluent A: water+0.1% formic acid; Eluent B: acetonitrile; gradient: 0-6 min 15-55% B, 6-8 min 55-100% B; rate 150 mL/min, temperature 25° C.

LC-MS (Method 1): Rₜ=0.78 min: MS (ESIpos): m/z=631 [M+H]⁺.

¹H-NMR (400 MHz, DMSO-d6) δ [ppm]: 1.232 (2.15), 1.247 (2.15), 1.291 (1.27), 1.328 (1.83), 1.455 (1.75), 1.472 (2.15), 1.903 (1.67), 1.921 (2.15), 1.939 (1.59), 2.701 (3.34), 2.719 (2.31), 2.729 (6.05), 2.888 (6.69), 3.336 (16.00), 4.431 (1.99), 4.449 (3.74), 4.464 (1.83), 4.829 (4.78), 4.848 (4.86), 5.113 (0.96), 5.135 (2.47), 5.157 (2.39), 7.345 (1.67), 7.362 (2.31), 7.366 (2.15), 7.383 (2.23), 7.441 (2.55), 7.454 (2.55), 7.486 (3.58), 7.503 (2.71), 7.736 (7.48), 7.758 (7.96), 7.901 (5.81), 7.923 (4.46), 7.950 (1.11), 8.217 (6.77), 8.343 (5.89), 8.415 (5.41), 8.506 (3.18), 8.518 (3.10), 8.623 (4.86), 8.711 (3.66).

### Final Intermediate 40-2

### N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide

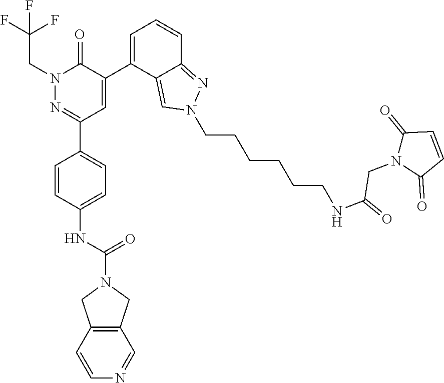 [SMILES: O=C(CN1C(=O)C=CC1=O)NCCCCCCn1cc2c(-c3cc(-c4ccc(NC(=O)N5Cc6ccncc6C5)cc4)nn(CC(F)(F)F)c3=O)cccc2n1]

A mixture of N-(4-{5-[2-(6-aminohexyl)-2H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide (11.3 mg, 17.9 μmol), 1-{2-[(2,5-dioxopyrrolidin-1-yl)oxy]-2-oxoethyl}-1H-pyrrole-2,5-dione (4.52 mg, 17.9 μmol) and N,N-diisopropylethylamine (6.2 μl, 36 μmol) in DMF (0.34 mL) was stirred at r.t. for 30 min under argon. Then formic acid (1.4 μl, 36 μmol) was added and the mixture was concentrated under vacuum. The crude product was purified by preparative HPLC to give 8.00 mg (65% purity, 38% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: YMC-Actus-ODS-AQ-HG 10 μm 150×20 mm; Eluent A: water+0.1% formic acid; Eluent B: acetonitrile; gradient: 0-6 min 15-55% B, 6-8 min 55-100% B; rate 60 mL/min, temperature 25° C.

LC-MS (Method 1): Rₜ=0.96 min; MS (ESipos): m/z=768 [M+H]⁺.

¹H-NMR (400 MHz, DMSO-d6) δ [ppm]: 1.231 (1.99), 1.262 (2.33), 1.353 (1.24), 1.362 (1.24), 1.892 (1.10), 1.910 (1.37), 1.926 (0.96), 2.331 (2.88), 2.539 (1.24), 2.646 (0.41), 2.673 (2.88), 2.678 (1.24), 2.989 (0.82), 3.006 (1.79), 3.021 (1.72), 3.357 (0.48), 3.965 (7.55), 4.417 (1.44), 4.434 (2.33), 4.451 (1.17), 4.827 (3.09), 4.846 (2.75), 5.106 (0.76), 5.129 (1.72), 5.151 (1.51), 5.173 (0.48), 7.065 (16.00), 7.338 (1.51), 7.355 (1.85), 7.359 (1.51), 7.377 (1.79), 7.440 (1.65), 7.452 (1.65), 7.483 (2.47), 7.499 (1.92), 7.730 (3.85), 7.737 (2.95), 7.752 (4.05), 7.759 (2.20), 7.897 (4.12), 7.919 (2.95), 8.057 (0.62), 8.071 (1.10), 8.085 (0.55), 8.216 (5.01), 8.425 (3.57), 8.503 (2.06), 8.515 (1.85), 8.620 (2.88), 8.677 (2.61).

### Example 40A

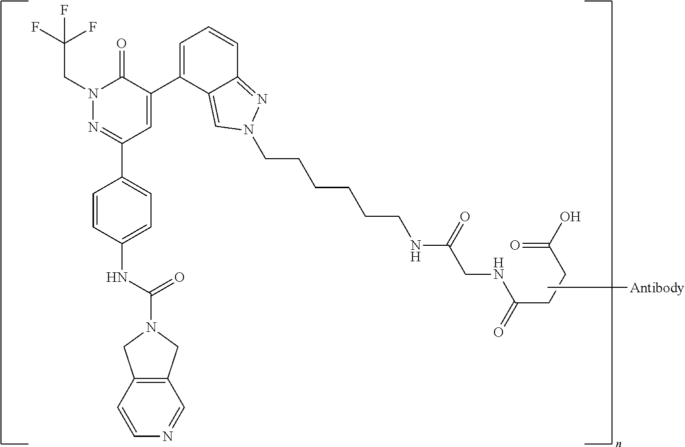

5 mg of anti-HER2 TPP-1015 (c=12.2 mg/mL) were coupled with N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate, 40-2, 310 μg, 65% purity, 0.27 μmol) according procedure 2 and the reaction was, after Sephadex purification, concentrated by ultracentrifugation and rediluted with PBS.

Protein concentration: 1.70 mg/mL

Drug/mAb ratio: 3.5 (LC-MS)

### Example 40B

5 mg of anti-CXCR5 TPP-9574 (c=10.65 mg/mL) were coupled with N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 40-2, 310 μg, 65% purity, 0.27 μmol) according procedure 2 and the reaction was, after Sephadex purification, concentrated by ultracentrifugation and rediluted with PBS.

Protein concentration: 0.26 mg/mL

Drug/mAb ratio: 5.5 (LC-MS)

### Example 40D

5 mg of anti-C4.4a TPP-509 (c=9.87 mg/mL) were coupled with N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 40-2, 310 μg, 65% purity, 0.27 μmol) according to procedure 2 and the reaction was, after Sephadex purification, concentrated by ultracentrifugation and rediluted with PBS.

Protein concentration: 0.91 mg/mL

Drug/mAb ratio: 4.7 (LC-MS)

### Example 40E

5 mg of anti-C4.4a TPP-668 (c=11.62 mg/mL) were coupled with N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide (see Final Intermediate 40-2, 310 μg, 65% purity, 0.27 μmol) according procedure 2 and the reaction was, after Sephadex purification, concentrated by ultracentrifugation and rediluted with PBS.

Protein concentration: 0.78 mg/mL

Drug/mAb ratio: 3.3 (LC-MS)

### Intermediate 41-1

### N²-{[(9H-Fluoren-9-yl)methoxy]carbonyl}-N⁶-(20-oxo-2,5,8,11,14,17-hexaoxaicosan-20-yl)-L-lysine

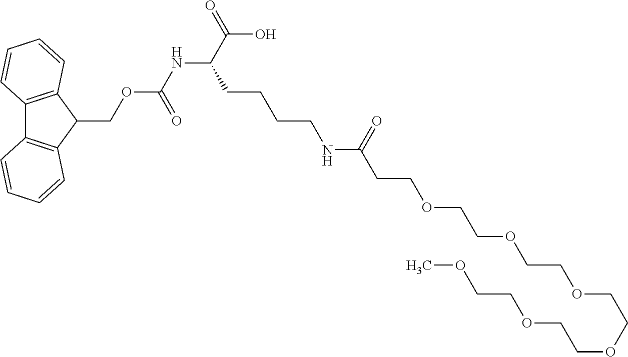 [SMILES: COCCOCCOCCOCCOCCOCCC(=O)NCCCC[C@H](NC(=O)OCC1c2ccccc2-c2ccccc21)C(=O)O]

To 1-[(20-oxo-2,5,8,11,14,17-hexaoxaicosan-20-yl)oxy]pyrrolidine-2,5-dione (CAS 1449390-12-8, 500 mg, 1.19 mmol) in DMF (10 mL) was added N²-[(9H-fluoren-9-yl)methyl]-L-lysine (CAS 105047-45-8, 481 mg, 1.31 mmol) and N,N-diisopropylethylamine (720 μl, 4.2 mmol) The mixture was stirred for 30 h at r.t., then diluted with formic acid (160 μl, 4.2 mmol) in DMSO (10 mL) and purified by preparative HPLC to give to give 522 mg (90% purity, 59% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm; Eluent A: water+0.1% formic acid; Eluent B: acetonitrile; gradient: 0-8 min 5-60% B, 8-11 min 60% B, rate 150 mL/min, temperature 25° C.

LC-MS (Method 1): Rₜ=1.09 min; MS (ESIpos): m/z=675 [M+H]⁺.

¹H-NMR (400 MHz, DMSO-d6) δ [ppm]: 1.26-1.42 (m, 4H), 1.54-1.73 (m, 2H), 2.28-2.31 (t, 2H), 2.52-2.54 (m, 1H), 2.98-3.06 (m, 2H), 3.23 (s, 3H), 3.40-3.51 (m, 20H), 3.57 (t, 2H), 3.86-3.93 (m, 1H), 4.19-4.29 (m, 3H), 7.33 (t, 2H), 7.42 (t, 2H), 7.61 (d, 1H), 7.73 (d, 2H), 7.82 (t, 1H), 7.90 (d, 2H), 12.59 (br s, 1H).

### Intermediate 41-2

### (9H-Fluoren-9-yl)methyl {(26S)-34-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-20,27-dioxo-2,5,8,11,14,17-hexaoxa-21,28-diazatetratriacontan-26-yl}carbamate

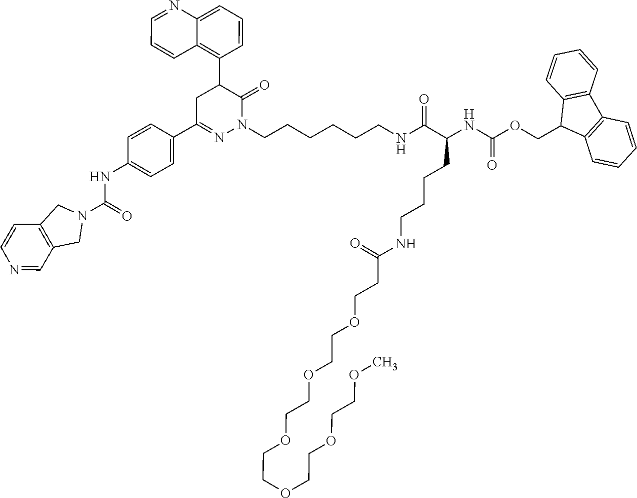 [SMILES: COCCOCCOCCOCCOCCOCCC(=O)NCCCC[C@H](NC(=O)OCC1c2ccccc2-c2ccccc21)C(=O)NCCCCCCN1N=C(c2ccc(NC(=O)N3Cc4ccncc4C3)cc2)CC(c2cccc3ncccc23)C1=O]

To N²-{[(9H-fluoren-9-yl)methoxy]carbonyl}-N⁶-(20-oxo-2,5,8,11,14,17-hexaoxaicosan-20-yl)-L-lysine (202 mg, 299 μmol) in DMF (0.9 mL) wad added 4-methylmorpholin (63 μl, 580 μmol) and HATU (110 mg, 288 μmol) and the mixture was stirred for 30 min. at r.t. Then the mixture was added to a suspension of N-{4-[1-(6-aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (Example 3, 120 mg, 214 μmol) in DMF (1.23 mL) and stirring was continued at r. t. for 30 min. Then formic acid (22 μl, 580 μmol) was added and the reaction mixture was diluted with and DMSO. Purification by preparative HPLC yielded 123 mg (85% purity, 40% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μm 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 2500.

LC-MS (Method 1): Rt=1.05 min; MS (ESIpos): m/z=610 [M+2H]²⁺.

¹H-NMR (400 MHz, DMSO-d6) δ [ppm]: 1.232 (0.79), 1.320 (0.57), 1.686 (0.23), 2.074 (0.68), 2.253 (0.23), 2.270 (0.57), 2.285 (0.34), 2.998 (0.23), 3.216 (6.13), 3.365 (1.25), 3.370 (0.91), 3.391 (0.68), 3.400 (0.79), 3.406 (0.79), 3.414 (1.13), 3.450 (1.36), 3.468 (5.56), 3.476 (10.21), 3.493 (0.45), 3.544 (0.34), 3.561 (0.68), 3.577 (0.34), 3.811 (0.23), 4.207 (0.34), 4.231 (0.34), 4.243 (0.34), 4.794 (0.57), 4.811 (0.57), 7.286 (0.23), 7.305 (0.57), 7.324 (0.34), 7.382 (0.45), 7.401 (0.68), 7.419 (0.68), 7.432 (0.57), 7.450 (0.34), 7.533 (0.23), 7.543 (0.23), 7.555 (0.23), 7.566 (0.23), 7.624 (0.57), 7.647 (0.91), 7.688 (0.34), 7.707 (1.13), 7.728 (0.91), 7.794 (0.23), 7.829 (0.23), 7.866 (0.57), 7.884 (0.45), 7.943 (0.34), 7.963 (0.34), 8.149 (0.23), 8.490 (0.45), 8.502 (0.45), 8.581 (0.23), 8.601 (0.79), 8.622 (0.57), 8.906 (0.34), 8.913 (0.34).

### Intermediate 41-3

### N-{4-[6-oxo-1-(6-{[N⁶-(20-oxo-2,5,8,11,14,17-hexaoxaicosan-20-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide

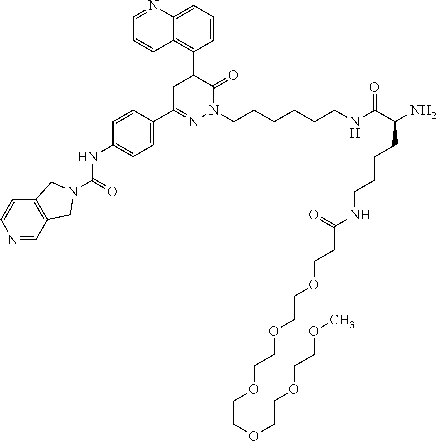 [SMILES: COCCOCCOCCOCCOCCOCCC(=O)NCCCC[C@H](N)C(=O)NCCCCCCN1N=C(c2ccc(NC(=O)N3Cc4ccncc4C3)cc2)CC(c2cccc3ncccc23)C1=O]

To 9H-fluoren-9-yl)methyl {(26S)-34-[3-{4-[(1,3-dihydro-2H-pyrrolo[3,4-c]pyridine-2-carbonyl) amino]phenyl}-6-oxo-5-(quinolin-5-yl)-5,6-dihydropyridazin-1(4H)-yl]-20,27-dioxo-2,5,8,11,14,17-hexaoxa-21,28-diazatetratriacontan-26-yl}carbamate (120 mg, 98.5 μmol) in DMF (640 μl) was added piperidine (160 μl, 1.6 mmol) and the mixture was stirred for 20 min at room temperature. Then formic acid (63 μl, 1.7 mmol) was added and the reaction mixture was diluted with DMSO. Purification by preparative HPLC yielded 85.2 mg (90% purity, 78% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rt=0.72 min; MS (ESIpos): m/z=997 [M+H]⁺ (ESIneg): m/z=995 [M−H]⁻.

¹H-NMR (400 MHz, DMSO-d6) δ [ppm]: 1.234 (0.21), 1.251 (0.21), 1.268 (0.21), 1.328 (1.04), 1.392 (0.35), 1.408 (0.35), 1.463 (0.21), 1.577 (0.21), 1.589 (0.42), 1.604 (0.48), 1.617 (0.35), 1.703 (0.28), 2.253 (0.48), 2.269 (1.04), 2.286 (0.48), 2.678 (0.21), 2.965 (0.21), 2.982 (0.48), 2.997 (0.48), 3.014 (0.21), 3.038 (0.28), 3.053 (0.35), 3.069 (0.28), 3.121 (0.21), 3.221 (7.41), 3.318 (0.76), 3.406 (3.67), 3.413 (2.42), 3.420 (2.29), 3.436 (0.76), 3.443 (0.83), 3.450 (1.32), 3.454 (1.87), 3.459 (1.87), 3.463 (1.32), 3.470 (0.97), 3.478 (8.80), 3.485 (16.00), 3.501 (0.69), 3.545 (0.55), 3.561 (1.25), 3.577 (0.55), 3.803 (0.14), 3.822 (0.35), 3.842 (0.35), 4.107 (0.21), 4.746 (0.21), 4.765 (0.28), 4.774 (0.28), 4.794 (0.90), 4.815 (0.83), 7.276 (0.21), 7.279 (0.21), 7.295 (0.62), 7.298 (0.55), 7.313 (0.42), 7.316 (0.35), 7.352 (0.35), 7.369 (0.48), 7.387 (0.21), 7.423 (0.48), 7.438 (0.62), 7.457 (0.48), 7.544 (0.42), 7.554 (0.42), 7.565 (0.42), 7.575 (0.42), 7.621 (0.83), 7.644 (1.18), 7.661 (0.55), 7.680 (0.48), 7.698 (0.48), 7.707 (1.25), 7.719 (0.62), 7.729 (0.76), 7.738 (0.42), 7.772 (0.21), 7.785 (0.35), 7.799 (0.21), 7.844 (0.69), 7.863 (0.76), 7.950 (0.55), 7.970 (0.48), 8.488 (0.62), 8.501 (0.55), 8.601 (0.97), 8.614 (0.35), 8.632 (0.76), 8.908 (0.48), 8.912 (0.48), 8.919 (0.48), 8.922 (0.42).

### Final Intermediate 41-4

### N-{4-[1-[6-({N²-[(2,5-Dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide

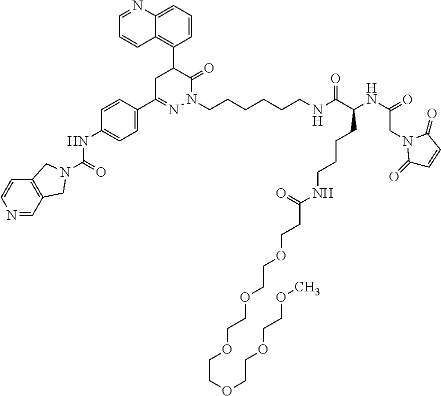 [SMILES: COCCOCCOCCOCCOCCOCCC(=O)NCCCC[C@H](NC(=O)CN1C(=O)C=CC1=O)C(=O)NCCCCCCN1N=C(c2ccc(NC(=O)N3Cc4ccncc4C3)cc2)CC(c2cccc3ncccc23)C1=O]

A mixture of N-{4-[6-oxo-1-(6-{[N-(20-oxo-2,5,8,11,14,17-hexaoxaicosan-20-yl)-L-lysyl]amino}hexyl)-5-(quinolin-5-yl)-1,4,5,6-tetrahydropyridazin-3-yl]phenyl}-1,3-dihydro-2H-pyrrolo [3,4-c]pyridine-2-carboxamide (40.0 mg, 38.1 μmol), 1-{2-[(2,5-dioxopyrrolidin-1-yl)oxy]-2-oxoethyl}-1H-pyrrole-2,5-dione (10.6 mg, 42.0 μmol) and N,N-diisopropylethylamine (13 μl, 76 μmol) in DMF (0.73 mL) was stirred at r.t. for 30 min under argon. Then the reaction was diluted with formic acid (2.9 μl, 76 μmol) in toluene (50 mL) and the mixture was concentrated under vacuum. The crude product was purified by column chromatography (SiO₂, dichloromethane/isopropyl alcohol, gradient containing 10% DMSO) to give 27.1 mg (95% purity, 60% yield) of the title compound.

LC-MS (Method 1): Rt=0.78 min; MS (ESIpos): m/z=576.5 [M+2H]²⁺, (ESIneg): m/z=1132 [M−H]⁻.

¹H-NMR (400 MHz, DMSO-d6) δ [ppm]: 1.17-1.28 (m, 3H), 1.28-1.49 (m, 10H), 1.59 (br s, 1H), 1.70 (br s, 2H), 2.27 (t, 2H), 2.33-2.34 (m, 1H), 2.52-2.54 (m, 10H), 2.67-2.68 (m, 1H), 2.69-2.70 (m, 1H), 2.94-3.09 (m, 4H), 3.22 (s, 3H), 3.36-3.58 (m, 24H), 3.75-3.90 (m, 2H), 4.03-4.19 (m, 3H), 4.74-4.74 (m, 1H), 4.74-4.85 (m, 4H), 7.02-7.12 (m, 1H), 7.07 (s, 1H), 7.42-7.47 (m, 2H), 7.52-7.59 (m, 1H), 7.62-7.66 (m, 2H), 7.69-7.79 (m, 4H), 7.89-7.98 (m, 2H), 8.27-8.35 (m, 1H), 8.46-8.52 (m, 1H), 8.58-8.64 (m, 3H), 8.92 (dd, 1H).

### Example 41A

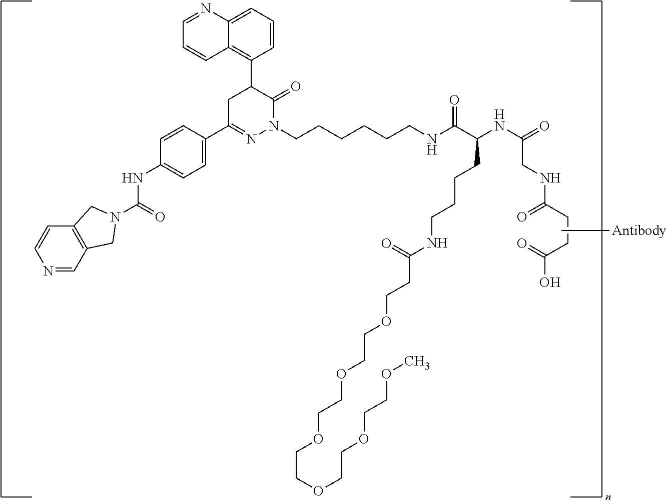

5 mg of anti-HER2 TPP-1015 (c=12.2 mg/mL) were coupled with N-{4-[1-[6-({N²-[(2,5-dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Final Intermediate 41-4, 320 μg, 95% purity, 0.27 μmol) according procedure 2 and the reaction was, after Sephadex purification, concentrated by ultracentrifugation and rediluted with PBS.

Protein concentration: 0.56 mg/mL

Drug/mAb ratio: 4.8 (UV)

### Intermediate 42-1

### N²-{[(9H-Fluoren-9-yl)methoxy]carbonyl}-N⁶-(14-oxo-2,5,8,11-tetraoxatetradecan-14-yl)-L-lysine

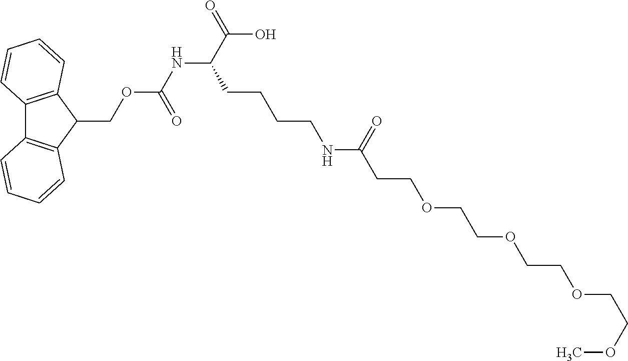 [SMILES: COCCOCCOCCOCCC(=O)NCCCC[C@H](NC(=O)OCC1c2ccccc2-c2ccccc21)C(=O)O]

To 1-[(14-oxo-2,5,8,11-tetraoxatetradecan-14-yl)oxy]pyrrolidine-2,5-dione (CAS 622405-78-1, 1.00 g, 3.00 mmol) in DMF (21 mL) was added N²-[(9H-fluoren-9-yl)methyl]-L-lysine (CAS 105047-45-8, 1.22 g, 3.30 mmol) and N,N-diisopropylethylamine (1.8 mL, 10 mmol) The mixture was stirred for 1 h at r.t., then diluted with formic acid (400 μl, 10 mmol) in DMSO (18 mL) and purified by preparative HPLC to give to give 1.49 g (95% purity, 80% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-8 min 5-60% B, 8-11 min 60% B, rate 150 mL/min, temperature 25° C.

LC-MS (Method 1): Rₜ=1.08 min; MS (ESIpos): m/z=587 [M+H]⁺.

1H-NMR (400 MHz, DMSO-d6) δ [ppm]: 1.23-1.42 (m, 4H), 1.52-1.74 (m, 2H), 2.24-2.31 (m, 2H), 2.97-3.06 (m, 2H), 3.22 (s, 3H), 3.39-3.43 (m, 2H), 3.43-3.50 (m, 10H), 3.58 (t, 2H), 3.86-3.93 (m, 1H), 4.19-4.30 (m, 3H), 7.33 (t, 2H), 7.42 (t, 2H), 7.62 (d, 1H), 7.73 (d, 2H), 7.82 (br t, 1H), 7.90 (d, 2H), 12.57 (br s, 1H).

### Intermediate 42-2

### (9H-Fluoren-9-yl)methyl {(20S)-28-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-14,21-dioxo-2,5,8,11-tetraoxa-15,22-diazaoctacosan-20-yl}carbamate

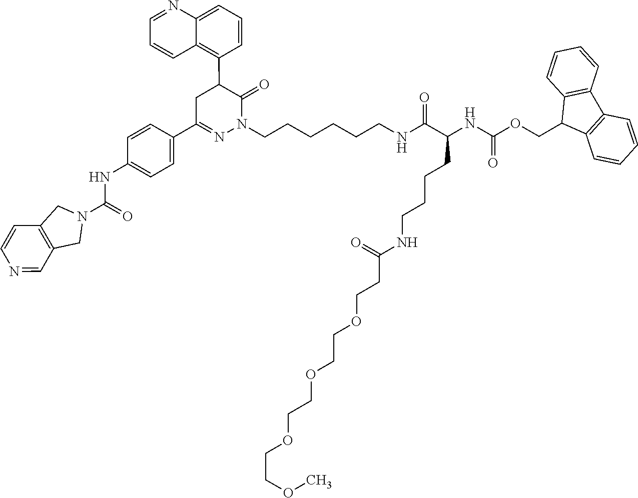 [SMILES: COCCOCCOCCOCCC(=O)NCCCC[C@H](NC(=O)OCC1c2ccccc2-c2ccccc21)C(=O)NCCCCCCN1N=C(c2ccc(NC(=O)N3Cc4ccncc4C3)cc2)CC(c2cccc3ncccc23)C1=O]

To N²-{[(9H-fluoren-9-yl)methoxy]carbonyl}-N⁶-(14-oxo-2,5,8,11-tetraoxatetradecan-14-yl)-L-lysine (175 mg, 299 μmol) in DMF (0.9 mL) wad added 4-methylmorpholin (63 μl, 580 μmol) and HATU (110 mg, 288 μmol) and the mixture was stirred for 20 min. at r.t. Then the mixture was added to a suspension of N-{4-[1-(6-aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Example 3, 120 mg, 214 μmol) in DMF (1.2 mL) and stirring was continued at r. t. for 30 min. Then formic acid (22 μl, 580 μmol) was added and the reaction mixture was diluted with and DMSO. Purification by preparative HPLC yielded 115 mg (95% purity, 45% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rₜ=1.04 min; MS (ESIpos): m/z=566 [M+2H]²⁺.

¹H-NMR (400 MHz, DMSO-d6) δ [ppm]: 1.320 (0.70), 1.380 (0.35), 1.395 (0.33), 1.688 (0.24), 2.253 (0.31), 2.269 (0.68), 2.285 (0.36), 2.998 (0.33), 3.008 (0.33), 3.021 (0.30), 3.038 (0.26), 3.056 (0.21), 3.210 (5.78), 3.222 (0.26), 3.382 (1.05), 3.393 (1.34), 3.399 (1.02), 3.406 (1.36), 3.419 (0.39), 3.449 (1.93), 3.464 (6.34), 3.475 (1.18), 3.484 (0.57), 3.545 (0.42), 3.561 (0.81), 3.577 (0.40), 3.811 (0.24), 3.830 (0.24), 3.887 (0.17), 3.901 (0.17), 4.193 (0.28), 4.208 (0.47), 4.231 (0.38), 4.244 (0.41), 4.262 (0.21), 4.755 (0.18), 4.764 (0.18), 4.793 (0.69), 4.811 (0.69), 7.287 (0.31), 7.306 (0.70), 7.324 (0.45), 7.382 (0.46), 7.400 (0.82), 7.419 (0.83), 7.433 (0.72), 7.451 (0.35), 7.533 (0.29), 7.543 (0.29), 7.555 (0.29), 7.565 (0.28), 7.626 (0.63), 7.648 (0.98), 7.689 (0.32), 7.707 (1.40), 7.729 (1.00), 7.798 (0.27), 7.816 (0.20), 7.832 (0.27), 7.846 (0.17), 7.865 (0.66), 7.884 (0.60), 7.943 (0.44), 7.964 (0.36), 8.183 (0.72), 8.490 (0.44), 8.502 (0.43), 8.581 (0.28), 8.601 (0.91), 8.624 (0.64), 8.904 (0.36), 8.907 (0.38), 8.914 (0.37), 8.917 (0.33).

### Intermediate 42-3

### N-{4-[6-oxo-1-(6-{[N⁶-(14-oxo-2,5,8,11-tetraoxatetradecan-14-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide

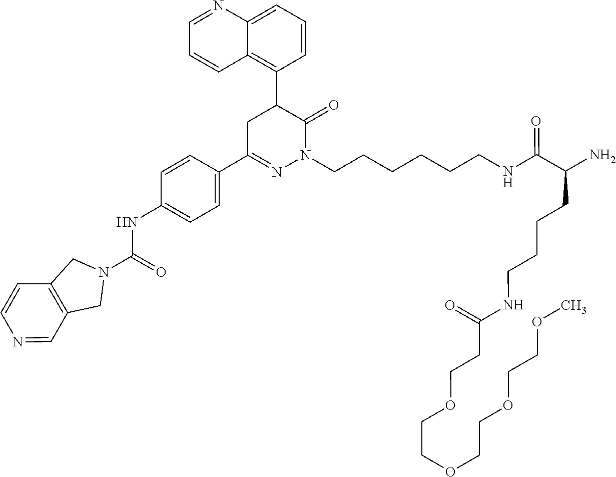 [SMILES: COCCOCCOCCOCCC(=O)NCCCC[C@H](N)C(=O)NCCCCCCN1N=C(c2ccc(NC(=O)N3Cc4ccncc4C3)cc2)CC(c2cccc3ncccc23)C1=O]

To (9H-fluoren-9-yl)methyl {(20S)-28-[3-{4-[(1,3-dihydro-2H-pyrrolo[3,4-c]pyridine-2-carbonyl) amino]phenyl}-6-oxo-5-(quinolin-5-yl)-5,6-dihydropyridazin-1(4H)-yl]-14,21-dioxo-2,5,8,11-tetraoxa-15,22-diazaoctacosan-20-yl}carbamate (113 mg, 100 μmol) in DMF (650 μl) and was added piperidine (160 μl, 1.6 mmol) and the mixture was stirred for 20 min at r.t. Then formic acid (64 μl. 1.7 mmol) was added and the reaction mixture was diluted with DMSO. Purification by preparative HPLC yielded 67.3 mg (90% purity, 67% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rₜ=0.65 min; MS (ESIpos): m/z=909 [M+H]⁺, (ESIneg): m/z=907 [M−H]⁻.

¹H-NMR (400 MHz, DMSO-d6) δ [ppm]: 1.328 (1.72), 1.391 (0.47), 1.407 (0.56), 1.423 (0.39), 1.477 (0.36), 1.591 (0.59), 1.605 (0.74), 1.619 (0.47), 1.684 (0.39), 1.702 (0.50), 2.253 (0.83), 2.269 (1.81), 2.285 (0.86), 2.966 (0.33), 2.982 (0.77), 2.997 (0.80), 3.014 (0.39), 3.035 (0.44), 3.041 (0.44), 3.049 (0.59), 3.060 (0.68), 3.077 (0.59), 3.091 (0.36), 3.219 (16.00), 3.291 (0.74), 3.392 (4.95), 3.403 (3.91), 3.410 (2.99), 3.417 (3.41), 3.436 (0.98), 3.444 (1.24), 3.450 (2.13), 3.455 (3.20), 3.459 (3.35), 3.463 (2.16), 3.475 (14.96), 3.480 (2.25), 3.487 (1.90), 3.495 (1.04), 3.497 (1.01), 3.546 (1.01), 3.562 (2.16), 3.578 (0.95), 3.822 (0.56), 3.841 (0.56), 4.107 (0.36), 4.749 (0.33), 4.767 (0.44), 4.776 (0.41), 4.795 (1.45), 4.816 (1.30), 7.276 (0.39), 7.280 (0.41), 7.295 (0.98), 7.298 (1.01), 7.314 (0.68), 7.317 (0.62), 7.352 (0.50), 7.369 (0.77), 7.388 (0.36), 7.422 (0.74), 7.436 (0.83), 7.442 (0.74), 7.460 (0.77), 7.544 (0.68), 7.554 (0.71), 7.565 (0.68), 7.575 (0.68), 7.623 (1.27), 7.646 (1.99), 7.662 (0.83), 7.681 (0.74), 7.699 (0.80), 7.709 (2.10), 7.717 (0.92), 7.720 (1.01), 7.731 (1.21), 7.738 (0.80), 7.783 (0.56), 7.797 (0.39), 7.816 (0.53), 7.845 (0.89), 7.863 (0.80), 7.950 (0.92), 7.970 (0.74), 8.489 (1.07), 8.501 (1.01), 8.602 (1.54), 8.615 (0.62), 8.632 (1.27), 8.908 (0.80), 8.912 (0.83), 8.919 (0.83), 8.923 (0.74).

### Final Intermediate 42-4

### N-{4-[1-[6-({N²-[(2,5-dioxo-2,5-dihydro-1H-pyrrol-1-yl)acetyl]-N⁶-(14-oxo-2,5,8,11-tetraoxatetradecan-14-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide

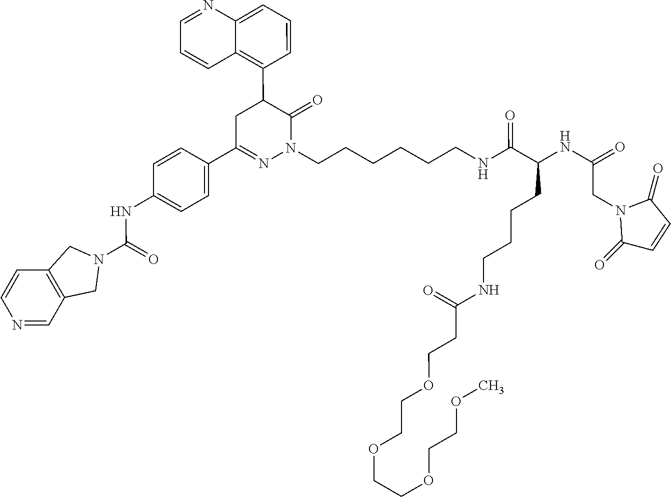 [SMILES: COCCOCCOCCOCCC(=O)NCCCC[C@H](NC(=O)CN1C(=O)C=CC1=O)C(=O)NCCCCCCN1N=C(c2ccc(NC(=O)N3Cc4ccncc4C3)cc2)CC(c2cccc3ncccc23)C1=O]

A mixture of N-{4-[6-oxo-1-(6-{[N6-(14-oxo-2,5,8,11-tetraoxatetradecan-14-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide (see Intermediate 42-3, 30.0 mg, 95% purity, 31.3 μmol), 1-{2-[(2,5-dioxopyrrolidin-1-yl)oxy]-2-oxoethyl}-1H-pyrrole-2,5-dione (8.69 mg, 34.4 μmol) and N,N-diisopropylethylamine (11 μl, 63 μmol) in DMF (600 μl) was stirred at r.t. for 30 min under argon. Then the reaction was diluted with formic acid (2.4 μl, 63 μmol) in toluene (50 mL) and the mixture was concentrated under vacuum. The crude product was purified by column chromatography (SiO₂, dichloromethane/isopropyl alcohol, gradient containing 10% DMSO) to give 17.5 mg (95% purity, 51% yield) of the title compound.

LC-MS (Method 1): Rₜ=0.75 min, MS (ESIpos): m/z=1046 [M+H]⁺, (ESIneg): m/z=1044 [M−H]⁻.

¹H-NMR (400 MHz, DMSO-d6) δ [ppm]: 1.326 (0.55), 1.345 (0.32), 1.366 (0.21), 1.382 (0.20), 1.399 (0.21), 1.701 (0.19), 2.257 (0.27), 2.274 (0.60), 2.290 (0.28), 2.968 (0.18), 2.983 (0.26), 2.994 (0.30), 3.014 (0.19), 3.220 (4.94), 3.394 (0.34), 3.404 (0.65), 3.411 (0.51), 3.418 (0.78), 3.445 (0.30), 3.452 (0.66), 3.456 (1.10), 3.459 (1.14), 3.463 (0.70), 3.475 (5.06), 3.480 (0.67), 3.487 (0.62), 3.496 (0.30), 3.548 (0.32), 3.565 (0.70), 3.581 (0.31), 3.824 (0.20), 3.841 (0.19), 4.074 (0.50), 4.088 (0.49), 4.770 (0.17), 4.779 (0.17), 4.798 (0.57), 4.818 (0.48), 7.071 (2.48), 7.424 (0.28), 7.437 (0.30), 7.444 (0.26), 7.462 (0.28), 7.541 (0.24), 7.552 (0.24), 7.563 (0.24), 7.573 (0.24), 7.626 (0.48), 7.649 (0.70), 7.700 (0.26), 7.712 (0.76), 7.717 (0.45), 7.720 (0.39), 7.734 (0.45), 7.739 (0.31), 7.773 (0.22), 7.906 (0.22), 7.950 (0.33), 7.970 (0.26), 8.289 (0.24), 8.309 (0.23), 8.491 (0.37), 8.504 (0.34), 8.593 (0.24), 8.604 (0.56), 8.614 (0.26), 8.625 (0.47), 8.909 (0.29), 8.913 (0.30), 8.920 (0.28), 8.923 (0.25).

### Example 42A

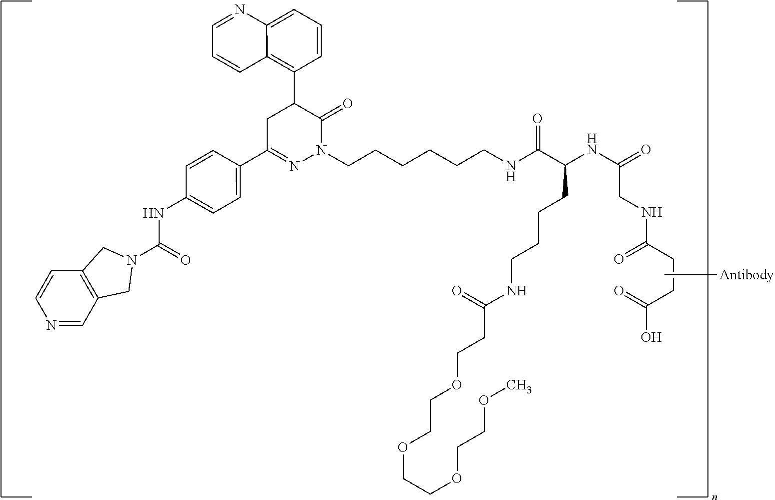

5 mg of anti-HER2 TPP-1015 (c=12.2 mg/mL) were coupled with N-{4-[1-[6-({N²-[(2,5-dioxo-2,5-dihydro-1H-pyrrol-1-yl)acetyl]-N⁶-(14-oxo-2,5,8,11-tetraoxatetradecan-14-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Final Intermediate 42-4, 290 μg, 95% purity, 0.27 μmol) according procedure 2 and the reaction was, after Sephadex purification, concentrated by ultracentrifugation and rediluted with PBS.

Protein concentration: 1.34 mg/mL

Drug/mAb ratio: 5.4 (UV)

### Example 42E

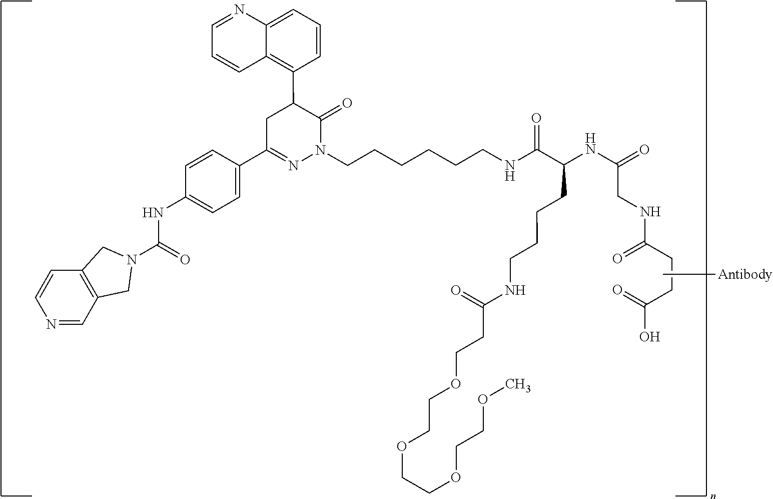

5 mg of anti-C4.4a TPP-668 (c=11.62 mg/mL) were coupled with N-{4-[1-[6-({N²-[(2,5-dioxo-2,5-dihydro-1H-pyrrol-1-yl)acetyl]-N⁶-(14-oxo-2,5,8,11-tetraoxa-tetradecan-14-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide (see Final Intermediate 42-4, 290 μg, 95% purity, 0.27 μmol) according procedure 2 and the reaction was, after Sephadex purification, concentrated by ultracentrifugation and rediluted with PBS.

Protein concentration: 1.40 mg/mL

Drug/mAb ratio: 4.3 (UV)

### Final Intermediate 43-1

### N-{5-[(2,5-Dioxopyrrolidin-1-yl)oxy]-5-oxopentanoyl}-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide

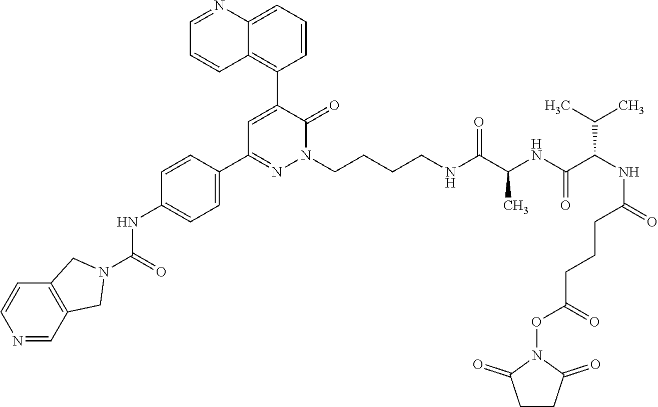 [SMILES: CC(C)[C@H](NC(=O)CCCC(=O)ON1C(=O)CCC1=O)C(=O)N[C@@H](C)C(=O)NCCCCn1nc(-c2ccc(NC(=O)N3Cc4ccncc4C3)cc2)cc(-c2cccc3ncccc23)c1=O]

A mixture of 1,1′-[(1,5-dioxopentane-1,5-diyl)bis(oxy)]di(pyrrolidine-2,5-dione) (12.3 mg, 37.6 μmol) and N,N-diisopropylethylamine (11 μl, 63 μmol) in DMF (0.1 mL) was stirred at r.t. for 30 min under argon, while L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide (see Intermediate 27-4, 22.0 mg, 31.3 μmol) in DMF (0.9 mL) was slowly added over that period. After additional stirring for 15 min the reaction was diluted with formic acid (2.4 μl, 63 μmol) in toluene (50 mL) and the mixture was concentrated under vacuum. The crude product was purified by column chromatography (SiO₂, dichloromethane/isopropyl alcohol, gradient) to give 7.00 mg (87% purity, 21% yield) of the title compound.

LC-MS (Method 1): Rₜ=0.72 min, MS (ESIpos): m/z=457.7 [M+2H]²⁺, (ESIneg): m/z=912 [M−H]⁻.

¹H-NMR (400 MHz, DMSO-d6) δ [ppm]: 0.770 (0.48), 0.789 (3.20), 0.806 (3.25), 0.817 (3.06), 0.835 (3.01), 1.027 (11.41), 1.035 (0.53), 1.042 (11.51), 1.053 (0.53), 1.176 (3.15), 1.194 (3.15), 1.233 (1.29), 1.256 (0.96), 1.318 (0.48), 1.334 (0.62), 1.486 (0.62), 1.506 (0.76), 1.526 (0.62), 1.796 (0.76), 1.815 (1.29), 1.834 (1.24), 1.937 (0.53), 1.954 (0.53), 2.246 (0.57), 2.252 (0.53), 2.264 (1.00), 2.271 (0.91), 2.283 (0.53), 2.790 (3.63), 4.117 (0.57), 4.134 (0.67), 4.138 (0.67), 4.155 (0.53), 4.199 (0.72), 4.217 (1.24), 4.235 (1.05), 4.822 (1.77), 4.839 (1.77), 6.984 (1.19), 7.437 (0.96), 7.450 (1.00), 7.506 (0.96), 7.516 (0.96), 7.528 (0.96), 7.538 (0.96), 7.670 (0.96), 7.674 (1.05), 7.688 (1.19), 7.691 (1.15), 7.706 (2.05), 7.729 (2.34), 7.837 (1.58), 7.859 (2.10), 7.877 (1.29), 7.906 (2.44), 7.928 (1.91), 7.967 (0.81), 7.986 (0.76), 8.078 (0.86), 8.099 (0.81), 8.119 (1.29), 8.140 (1.10), 8.161 (3.44), 8.501 (1.29), 8.513 (1.19), 8.618 (1.86), 8.643 (1.67), 8.938 (1.10), 8.941 (1.19), 8.948 (1.10), 8.952 (1.05).

### Example 43D

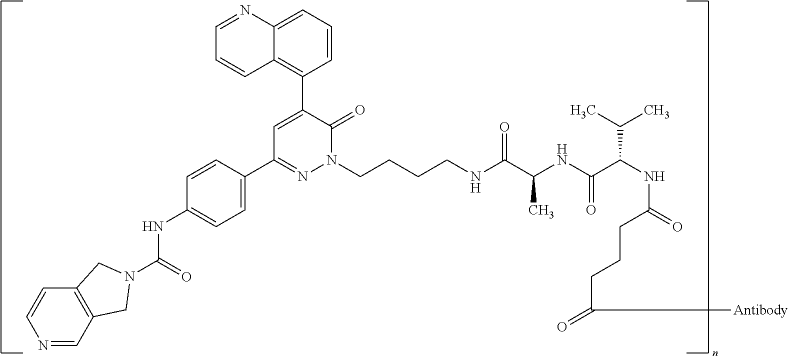

5 mg of anti-C4.4a TPP-509 (c=9.87 mg/mL) were coupled with N-{5-[(2,5-dioxopyrrolidin-1-yl)oxy]-5-oxopentanoyl}-L-valyl-N-{4-[3-{4-[(1,3-dihydro-2H-pyrrolo[3,4-c]pyridine-2-carbonyl)-amino]phenyl}-6-oxo-5-(quinolin-5-yl)pyridazin-1(6H)-yl]butyl}-L-alaninamide (see Final Intermediate 43-1, 350 μg, 87% purity, 0.33 μmol) according procedure 3 and the reaction was, after Sephadex purification, concentrated by ultracentrifugation and rediluted with PBS.

Protein concentration: 1.59 mg/mL

Drug/mAb ratio: 0.9 (UV)

### Example 43E

5 mg of anti-C4.4a TPP-668 (c=11.62 mg/mL) were coupled with N-{5-[(2,5-dioxopyrrolidin-1-yl)oxy]-5-oxopentanoyl}-L-valyl-N-{4-[3-{4-[(1,3-dihydro-2H-pyrrolo[3,4-c]pyridine-2-carbonyl)-amino]phenyl}-6-oxo-5-(quinolin-5-yl)pyridazin-1(6H)-yl]butyl}-L-alaninamide (see Final Intermediate 43-1, 350 μg, 87% purity, 0.33 μmol) according procedure 3 and the reaction was, after Sephadex purification, concentrated by ultracentrifugation and rediluted with PBS.

Protein concentration: 1.70 mg/mL

Drug/mAb ratio: 1,1 (UV)

### Intermediate 44-1

### Tert-Butyl {6-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]hexyl}carbamate

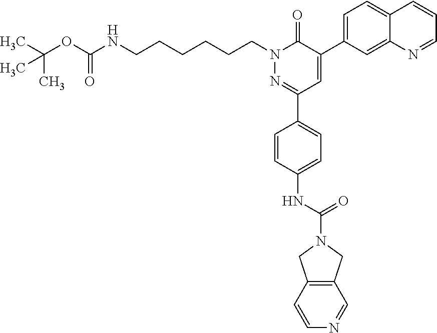 [SMILES: CC(C)(C)OC(=O)NCCCCCCn1nc(-c2ccc(NC(=O)N3Cc4ccncc4C3)cc2)cc(-c2ccc3cccnc3c2)c1=O]

Potassium carbonate (0.48 g, 3.47 mmol) was added to a mixture of N-{4-[6-oxo-5-(quinolin-7-yl)-1,6-dihydropyridazin-3-yl]phenyl}-1,3-dihydro-2H-pyrrolo[3,4-c]pyridine-2-carboxamide, (see Example 24, 800 mg, 1.74 mmol) in DMF (32 mL) and stirred for 10 mins. Tert-butyl (6-bromohexyl)carbamate (0.58 g 2.08 mmol) was then added and the mixture was stirred at r.t. for 48 hours. The mixture was poured into water and the resulting solid was collected by filtration to give the desired raw product as pale yellow solid. The precipitate was purified by purified by column chromatography (SiO₂, dichloromethane/methanol gradient) to give 0.55 g (95% purity, 45% yield) of the desired product.

LC-MS (Method 5): Rₜ=0.83 min; MS (ESIpos): m/z=660 [M+H]⁺

¹H NMR (300 MHz, DMSO-d6) δ [ppm]: 1.27-1.54 (m, 15H), 1.78-1.89 (m, 2H), 2.85-2.89 (m, 2H), 4.20-4.24 (m, 2H), 4.81 (d, 4H), 6.71-6.75 (m, 1H), 7.42 (d, 1H), 7.56 (q, 1H), 7.71 (d, 2H), 7.94 (d, 2H), 8.04 (d, 1H), 8.12 (dd, 1H), 8.28 (s, 1H), 8.39 (d, 1H), 8.48 (d, 1H), 8.60 (d, 2H), 8.67 (s, 1H), 8.94-8.95 (m, 1H).

### Intermediate 44-2

### N-{4-[1-(6-Aminohexyl)-6-oxo-5-(quinolin-7-yl)-1,6-dihydropyridazin-3-yl]phenyl}-1,3-dihydro-2H-pyrrolo[3,4-c]pyridine-2-carboxamide-hydrogen Chloride

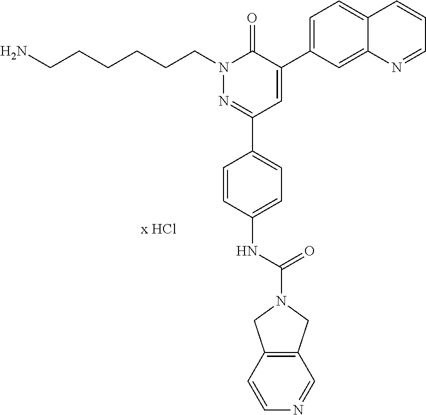 [SMILES: NCCCCCCn1nc(-c2ccc(NC(=O)N3Cc4ccncc4C3)cc2)cc(-c2ccc3cccnc3c2)c1=O]

A mixture of tert-butyl {6-[3-{4-[(1,3-dihydro-2H-pyrrolo[3,4-c]pyridin-2-ylcarbonyl)amino]phenyl}-6-oxo-5-(quinolin-7-yl)pyridazin-1(6H)-yl]hexyl}carbamate (300 mg 0.45 mmol) and HCl in cyclopentyl methyl ether (3M, 10 mL) and ethanol (1 mL) was stirred at r.t. for 2 h. The solid formed was collected by filtration, washed with heptane and dried to give 240 mg (96% purity, 83% yield) of the desired product as yellow solid.

LC-MS (Method 6): Rₜ=1.59 min; MS (ESIpos): m/z=560 [M+H]⁺

¹H NMR (300 MHz, DMSO-d6): δ (ppm)=1.35-1.42 (m, 4H), 1.52-1.55 (m, 2H), 1.80-1.87 (m, 2H), 2.69-2.78 (m, 2H), 4.23-4.28 (m, 2H), 4.95 (d, 4H), 7.71-7.77 (m, 6H), 7.94-7.97 (m, 3H), 8.17-8.23 (m, 2H), 8.35 (s, 1H), 8.65-8.68 (m, 1H), 8.77-8.84 (m, 3H), 8.91 (s, 1H), 9.07 (br s, 1H).

### Intermediate 44-3

### (9H-Fluoren-9-yl)methyl {(26S)-34-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]-20,27-dioxo-2,5,8,11,14,17-hexaoxa-21,28-diazatetratriacontan-26-yl}carbamate

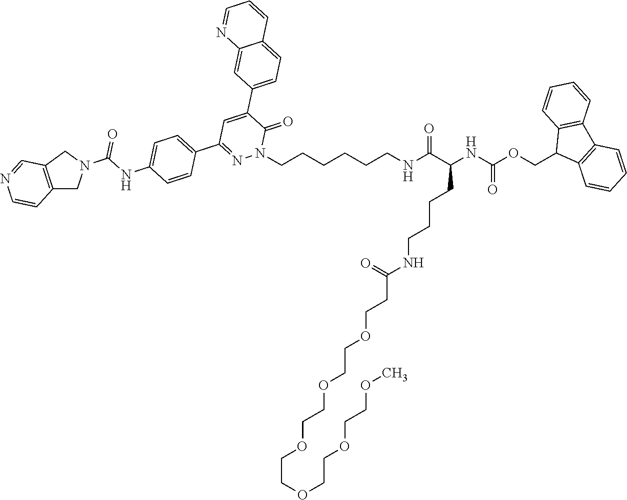 [SMILES: COCCOCCOCCOCCOCCOCCC(=O)NCCCC[C@H](NC(=O)OCC1c2ccccc2-c2ccccc21)C(=O)NCCCCCCn1nc(-c2ccc(NC(=O)N3Cc4ccncc4C3)cc2)cc(-c2ccc3cccnc3c2)c1=O]

To N²-{[(9H-fluoren-9-yl)methoxy]carbonyl}-N⁶-(20-oxo-2,5,8,11,14,17-hexaoxaicosan-20-yl)-L-lysine (see Intermediate 41-1, 179 mg, 266 μmol) in DMF (1.0 mL) wad added 4-methylmorpholin (83 μL, 760 μmol) and HATU (97.4 mg, 256 μmol) and the mixture was stirred for 30 min. at r.t. Then the mixture was added to a suspension of N-{4-[1-(6-aminohexyl)-6-oxo-5-(quinolin-7-yl)-1,6-dihydropyridazin-3-yl]phenyl}-1,3-dihydro-2H-pyrrolo[3,4-c]pyridine-2-carboxamide hydrogen chloride (see Intermediate 44-2, 120 mg, 190 μmol) in DMF (1.0 mL) and stirring was continued at r. t. for 30 min. Then formic acid (29 μL, 760 μmol) was added and the reaction mixture was diluted with and DMSO. Purification by preparative HPLC yielded 70.0 mg (90% purity, 27% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rt=1.09 min; MS (ESpos): m/z=1216.7 [M+H]⁺.

¹H-NMR (400 MHz, DMSO-d6) δ [ppm]: 1.225 (0.32), 1.239 (0.27), 1.252 (0.27), 1.263 (0.25), 1.287 (0.23), 1.359 (1.22), 1.399 (0.62), 1.415 (0.53), 1.498 (0.18), 1.521 (0.20), 1.533 (0.17), 1.544 (0.18), 1.558 (0.23), 1.577 (0.20), 1.812 (0.29), 1.828 (0.36), 2.072 (0.20), 2.246 (0.46), 2.262 (0.99), 2.278 (0.56), 2.327 (0.20), 2.518 (1.07), 2.523 (0.65), 2.539 (0.28), 2.669 (0.19), 2.995 (0.48), 3.005 (0.48), 3.021 (0.36), 3.037 (0.39), 3.052 (0.43), 3.067 (0.32), 3.084 (0.18), 3.211 (7.82), 3.225 (0.76), 3.384 (1.88), 3.394 (1.79), 3.401 (1.33), 3.408 (1.81), 3.423 (0.68), 3.442 (2.49), 3.461 (8.94), 3.469 (16.00), 3.487 (2.36), 3.537 (0.57), 3.552 (1.14), 3.569 (0.58), 3.886 (0.25), 3.899 (0.27), 4.167 (0.17), 4.185 (0.50), 4.202 (0.65), 4.233 (1.22), 4.253 (0.63), 4.827 (1.05), 4.845 (1.07), 7.272 (0.44), 7.290 (1.00), 7.309 (0.68), 7.363 (0.62), 7.381 (0.99), 7.393 (0.61), 7.399 (0.60), 7.414 (0.49), 7.445 (0.44), 7.457 (0.43), 7.580 (0.60), 7.591 (0.58), 7.601 (0.59), 7.611 (0.63), 7.684 (0.53), 7.699 (0.71), 7.717 (0.59), 7.728 (1.21), 7.750 (1.30), 7.779 (0.24), 7.792 (0.42), 7.807 (0.30), 7.820 (0.35), 7.840 (1.12), 7.859 (0.85), 7.951 (1.19), 7.974 (0.94), 8.053 (0.60), 8.074 (0.97), 8.125 (0.68), 8.129 (0.64), 8.147 (0.36), 8.150 (0.39), 8.309 (1.57), 8.414 (0.49), 8.433 (0.47), 8.508 (0.27), 8.623 (0.38), 8.656 (0.98), 8.694 (0.93), 8.963 (0.54), 8.967 (0.57), 8.974 (0.56), 8.978 (0.50).

### Intermediate 44-4

### N-{4-[6-oxo-1-(6-{[N⁶-(20-oxo-2,5,8,11,14,17-hexaoxaicosan-20-yl)-L-lysyl]amino}hexyl)-5-(quinolin-7-yl)-1,6-dihydropyridazin-3-yl]phenyl}-1,3-dihydro-2H-pyrrolo[3,4-c]pyridine-2-carboxamide

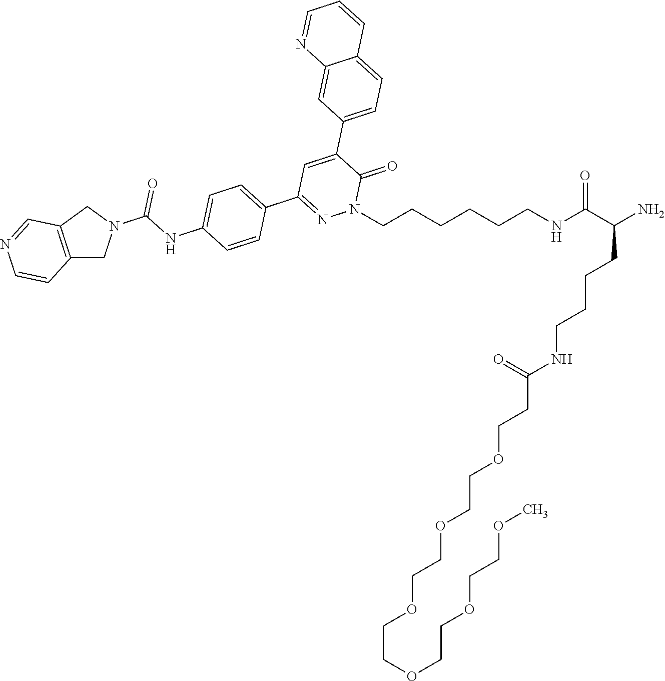 [SMILES: COCCOCCOCCOCCOCCOCCC(=O)NCCCC[C@H](N)C(=O)NCCCCCCn1nc(-c2ccc(NC(=O)N3Cc4ccncc4C3)cc2)cc(-c2ccc3cccnc3c2)c1=O]

To (9H-fluoren-9-yl)methyl {(26S)-34-[3-{4-[(1,3-dihydro-2H-pyrrolo[3,4-c]pyridine-2-carbonyl) amino]phenyl}-6-oxo-5-(quinolin-7-yl)pyridazin-1(6H)-yl]-20,27-dioxo-2,5,8,11,14,17-hexaoxa-21,28-diazatetratriacontan-26-yl}carbamate (72.0 mg, 59.2 μmol) in DMF (390 μL) and was added piperidine (97 μL, 980 μmol) and the mixture was stirred for 20 min at r.t. Then formic acid (38 μL, 1.0 mmol) was added and the reaction mixture was diluted with DMSO. Purification by preparative HPLC yielded 32.3 mg (90% purity, 49% yield) of the title compound.

HPLC: Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: 0-7.5 min 1-25% B, 7.5-9 min 25% B. 9-16 min 25-60% B; rate 150 mL/min, temperature 25° C.

LC-MS (Method 1): Rₜ=0.82 min; MS (ESIpos): m/z=994.7 [M+H]⁺.

¹H-NMR (400 MHz, DMSO-d6) δ [ppm]: 1.234 (0.20), 1.258 (0.21), 1.334 (0.33), 1.353 (0.46), 1.380 (0.65), 1.424 (0.42), 1.441 (0.39), 1.840 (0.23), 1.857 (0.30), 2.247 (0.43), 2.264 (0.93), 2.280 (0.44), 2.331 (0.39), 2.967 (0.24), 2.983 (0.46), 2.998 (0.47), 3.014 (0.25), 3.043 (0.22), 3.065 (0.33), 3.079 (0.41), 3.095 (0.34), 3.116 (0.23), 3.220 (8.48), 3.394 (2.04), 3.404 (2.43), 3.411 (2.22), 3.418 (2.68), 3.430 (1.64), 3.438 (1.74), 3.444 (2.05), 3.449 (2.55), 3.454 (2.55), 3.459 (2.00), 3.466 (1.74), 3.474 (8.82), 3.481 (16.00), 3.498 (1.32), 3.538 (0.99), 3.554 (1.51), 3.571 (0.84), 4.243 (0.29), 4.249 (0.31), 4.261 (0.45), 4.279 (0.26), 4.833 (0.72), 4.852 (0.72), 7.443 (0.40), 7.456 (0.40), 7.585 (0.50), 7.596 (0.46), 7.606 (0.47), 7.616 (0.49), 7.733 (0.84), 7.755 (0.95), 7.770 (0.17), 7.785 (0.30), 7.961 (1.06), 7.983 (0.99), 8.063 (0.46), 8.085 (0.74), 8.135 (0.56), 8.140 (0.53), 8.157 (0.30), 8.161 (0.32), 8.328 (1.34), 8.420 (0.34), 8.439 (0.32), 8.506 (0.46), 8.519 (0.45), 8.625 (0.70), 8.666 (0.67), 8.701 (0.63), 8.968 (0.42), 8.972 (0.44), 8.979 (0.42), 8.983 (0.40).

### Final Intermediate 44-5

### N-(4-{1-[6-({N²-[(2,5-dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-7-yl)-1,6-dihydropyridazin-3-yl}phenyl)-1,3-dihydro-2H-pyrrolo[3,4-c]pyridine-2-carboxamide

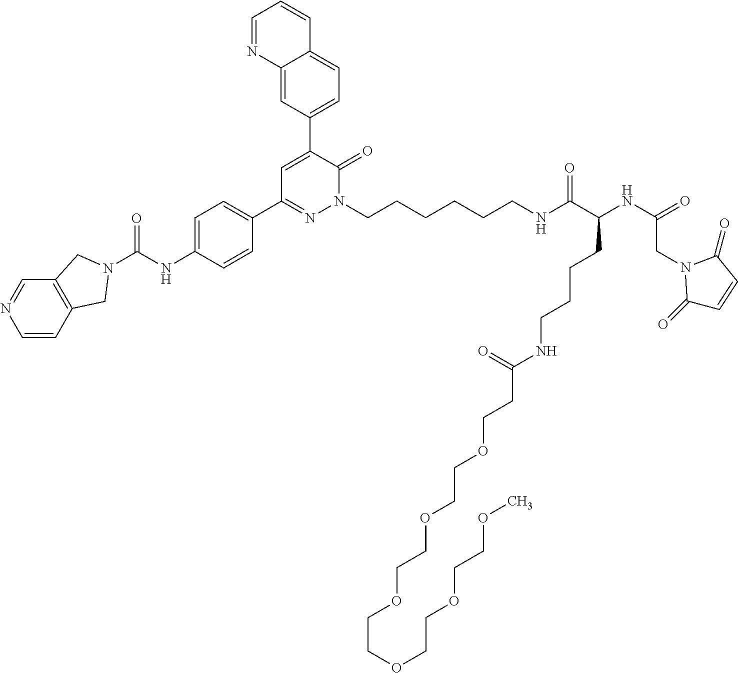 [SMILES: COCCOCCOCCOCCOCCOCCC(=O)NCCCC[C@H](NC(=O)CN1C(=O)C=CC1=O)C(=O)NCCCCCCn1nc(-c2ccc(NC(=O)N3Cc4ccncc4C3)cc2)cc(-c2ccc3cccnc3c2)c1=O]

A mixture of N-{4-[6-oxo-1-(6-{[N⁶-(20-oxo-2,5,8,11,14,17-hexaoxaicosan-20-yl)-L-lysyl]amino}hexyl)-5-(quinolin-7-yl)-1,6-dihydropyridazin-3-yl]phenyl}-1,3-dihydro-2H-pyrrolo[3,4-c]pyridine-2-carboxamide (21.0 mg, 20.1 μmol), 1-{2-[(2,5-dioxopyrrolidin-1-yl)oxy]-2-oxoethyl}-1H-pyrrole-2,5-dione (5.57 mg, 22.1 μmol) and N,N-diisopropylethylamine (7.0 μl, 40 μmol) in DMF (390 μL) was stirred at r.t. for 30 min under argon. Then the reaction was diluted with formic acid (1.5 μl, 40 μmol) in toluene (10 mL) and the mixture was concentrated under vacuum (azeotropic distillation with toluene was executed twice). The crude product was purified by column chromatography (SiO₂, dichloromethane/isopropyl alcohol, gradient containing 10% DMSO) to give 8.90 mg (80% purity, 31% yield) of the title compound.

LC-MS (Method 1): Rₜ=0.85 min, MS (ESIpos): m/z=1131.8 [M+H]⁺.

¹H-NMR (400 MHz, DMSO-d6) δ [ppm]: 1.145 (0.30), 1.232 (0.74), 1.364 (0.89), 1.591 (0.15), 1.852 (0.30), 2.249 (0.44), 2.265 (0.74), 2.281 (0.44), 2.728 (1.78), 2.980 (0.44), 2.994 (0.30), 3.219 (5.19), 3.283 (0.44), 3.293 (0.59), 3.377 (1.19), 3.394 (0.74), 3.404 (1.04), 3.410 (0.89), 3.418 (1.33), 3.452 (2.22), 3.473 (5.93), 3.481 (10.81), 3.497 (0.59), 3.538 (0.44), 3.554 (0.89), 3.571 (0.30), 4.029 (0.15), 4.072 (0.59), 4.087 (0.59), 4.129 (0.15), 4.162 (0.30), 4.256 (0.44), 4.834 (0.74), 4.851 (0.59), 7.064 (2.96), 7.443 (0.30), 7.456 (0.30), 7.584 (0.44), 7.594 (0.44), 7.604 (0.30), 7.615 (0.44), 7.731 (0.74), 7.752 (1.04), 7.914 (0.30), 7.950 (0.44), 7.961 (0.89), 7.983 (0.59), 8.062 (0.44), 8.083 (0.59), 8.136 (0.44), 8.140 (0.44), 8.162 (0.30), 8.287 (0.30), 8.309 (0.30), 8.323 (1.04), 8.418 (0.30), 8.439 (0.30), 8.505 (0.44), 8.518 (0.44), 8.624 (0.74), 8.656 (0.59), 8.697 (0.59), 8.967 (0.44), 8.972 (0.44), 8.978 (0.44), 8.983 (0.30).

### Example 44A

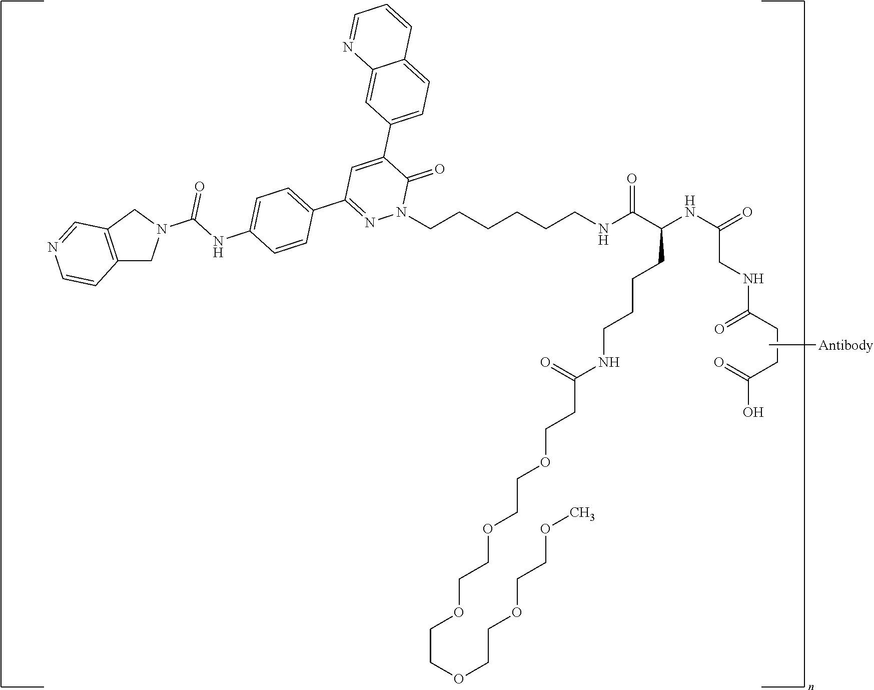

5 mg of anti-HER2 TPP-1015 (c=12.2 mg/mL) were coupled with N-(4-{1-[6-({N²-[(2,5-dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-7-yl)-1,6-dihydropyridazin-3-yl}phenyl)-1,3-dihydro-2H-pyrrolo[3,4-c]pyridine-2-carboxamide (see Final Intermediate 44-5, 380 μg, 80% purity, 0.27 μmol) according procedure 2 and the reaction was, after Sephadex purification, concentrated by ultracentrifugation and rediluted with PBS.

Protein concentration: 2.31 mg/mL

Drug/mAb ratio: 6.9 (UV)

### Intermediate 45-1

### Tert-Butyl {6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}carbamate

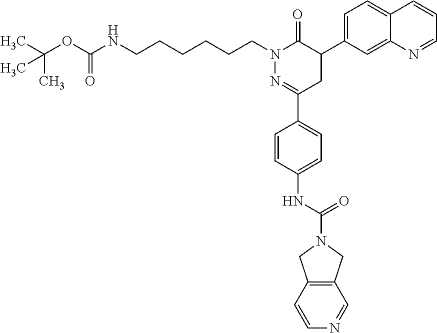 [SMILES: CC(C)(C)OC(=O)NCCCCCCN1N=C(c2ccc(NC(=O)N3Cc4ccncc4C3)cc2)CC(c2ccc3cccnc3c2)C1=O]

Tert-butyl {6-[3-{4-[(1,3-dihydro-2H-pyrrolo[3,4-c]pyridin-2-ylcarbonyl)amino]phenyl}-6-oxo-5-(quinolin-7-yl)pyridazin-1(6H)-yl]hexyl}carbamate (see Intermediate 44-1, 180 mg 0.273 mmol), in acetic acid, 9 mL was heated to 90° C. and zinc powder, 89.1 mg (1.36 mmol) was added. The mixture was heated at this temperature for another 1 hour. The reaction mixture was cooled to r.t., filtered through celite and concentrated under reduced pressure. The crude residue was purified by preparative HPLC (XSelect C18, 19×150 mm, 5 μm, 0.1% formic acid in water-acetonitrile, 20-33% over 10 min.) and lyophilized to give the desired product, 100 mg (55% yield).

LC-MS (Method 4): Rt=1.33 min, 100%. MS (ESIneg): m/z=(M−H)− 660.

¹H-NMR (400 MHz, CDCl₃) δ (ppm): 1.24-1.37 (m, 5H), 1.46 (s, 11H), 1.72-1.74 (m, 1H), 3.05-3.12 (m, 2H), 3.28-3.30 (m, 2H), 3.84-3.96 (m, 2H), 3.99-4.02 (m, 1H), 4.63 (br s, 1H), 4.84 (d, 4H), 6.72 (br s, 1H), 7.23-7.25 (m, 1H), 7.34-7.37 (m, 1H), 7.48-7.50 (m, 3H), 7.67 (dd, 2H), 7.79 (d, 1H), 7.91 (s, 1H), 8.11 (d, 1H), 8.53-8.56 (m, 2H), 8.84-8.85 (m, 1H).

### Example 45

### N-{4-[1-(6-Aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide hydrogen chloride

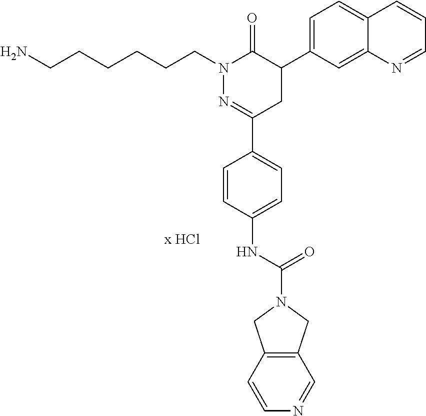 [SMILES: NCCCCCCN1N=C(c2ccc(NC(=O)N3Cc4ccncc4C3)cc2)CC(c2ccc3cccnc3c2)C1=O]

A mixture of tert-butyl {6-[3-{4-[(1,3-dihydro-2H-pyrrolo[3,4-c]pyridin-2-ylcarbonyl)amino]-phenyl}-6-oxo-5-(quinolin-7-yl)-5,6-dihydropyridazin-1(4H)-yl]hexyl}carbamate (see Intermediate 45-1, 170 mg, 0.257 mmol), in hydrogen chloride (3 M in cyclopentyl methyl ether, 20 mL), and ethanol (2 mL) was stirred at r.t. for 5 hours. The crude reaction mixture was combined with another batch of material starting from tert-butyl {6-[3-{4-[(1,3-dihydro-2H-pyrrolo[3,4-c]pyridin-2-ylcarbonyl)amino]phenyl}-6-oxo-5-(quinolin-7-yl)-5,6-dihydropyridazin-1(4H)-yl]hexyl}carbamate (50.0 mg, 75.6 μmol), and the solids collected by filtration, washed with heptane and dried to give 190 mg of the desired product, (97% purity, 88% yield).

LC-MS (Method 4): Rt=0.56 min, 96.7%. MS (ESIpos): m/z=(M+H)+ 562.

¹H NMR (300 MHz, DMSO-d6) δ (ppm): 1.31-1.44 (m, 4H), 1.49-1.54 (m, 2H), 1.65-1.66 (m, 1H), 2.62-2.82 (m, 2H), 3.21-3.67 (m, 3H), 3.68-3.88 (m, 2H), 4.17 (t, 1H), 4.86 (d, 4H), 7.51-7.67 (m, 6H), 7.72-7.76 (m, 4H), 7.87 (s, 1H), 7.99 (d, 1H), 8.42 (d, 1H), 8.61 (d, 1H), 8.73 (s, 2H), 8.89 (t, 1H).

### Final Intermediate 46-1

### N-{4-[1-{6-[2-(2,5-dioxo-2,5-dihydro-1H-pyrrol-1-yl)acetamido]hexyl}-6-oxo-5-(quinolin-7-yl)-1,4,5,6-tetrahydropyridazin-3-yl]phenyl}-1,3-dihydro-2H-pyrrolo[3,4-c]pyridine-2-carboxamide

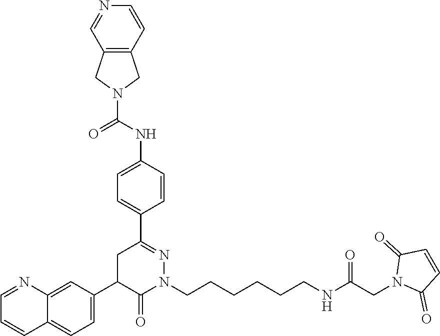 [SMILES: O=C(CN1C(=O)C=CC1=O)NCCCCCCN1N=C(c2ccc(NC(=O)N3Cc4ccncc4C3)cc2)CC(c2ccc3cccnc3c2)C1=O]

A mixture of N-{4-[1-(6-aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide hydrogen chloride (see Example 45, 15.0 mg, 23.6 μmol), maleimidoacetic acid N-hydroxysuccinimide ester (5.96 mg, 23.6 μmol), DIPEA (18 μl, 95 μmol) and DMF (360 μL) was stirred for 14 h at r.t. After that the mixture was filtered and purified by preparative HPLC to give the title compound (8.00 mg, 48% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18 10 μM 120×30 mm. Eluent A: water+0.1 Vol-% HCOOH; Eluent B: acetonitrile; gradient: 0-20 min 10-50% B. rate 150 mL/min, temperature 25° C.

LC-MS (Method 1): Rₜ=0.78 min; MS (ESpos): m/z=699 [M+H]⁺.

¹H-NMR (400 MHz, DMSO-d6) δ [ppm]: 1.231 (0.55), 1.294 (3.40), 1.352 (1.24), 1.368 (1.38), 1.678 (1.33), 2.518 (11.22), 2.523 (7.45), 2.994 (0.83), 3.011 (1.89), 3.025 (1.89), 3.041 (0.74), 3.358 (0.69), 3.384 (1.24), 3.401 (1.10), 3.411 (1.10), 3.434 (1.10), 3.776 (0.46), 3.791 (0.74), 3.809 (1.38), 3.829 (1.33), 3.847 (0.69), 3.863 (0.41), 3.986 (8.92), 4.150 (0.87), 4.170 (1.33), 4.188 (0.74), 4.807 (3.22), 4.825 (3.13), 7.086 (16.00), 7.427 (1.75), 7.440 (1.75), 7.489 (1.93), 7.499 (1.89), 7.509 (1.84), 7.520 (1.89), 7.556 (1.52), 7.561 (1.56), 7.578 (1.56), 7.582 (1.61), 7.646 (3.31), 7.668 (4.46), 7.752 (4.55), 7.774 (2.90), 7.848 (2.76), 7.939 (2.71), 7.960 (2.34), 8.071 (0.69), 8.085 (1.33), 8.098 (0.64), 8.320 (1.47), 8.338 (1.33), 8.493 (2.34), 8.505 (2.11), 8.607 (3.31), 8.638 (2.94), 8.860 (2.02), 8.864 (2.07), 8.870 (1.93), 8.875 (1.75).

### Example 46A

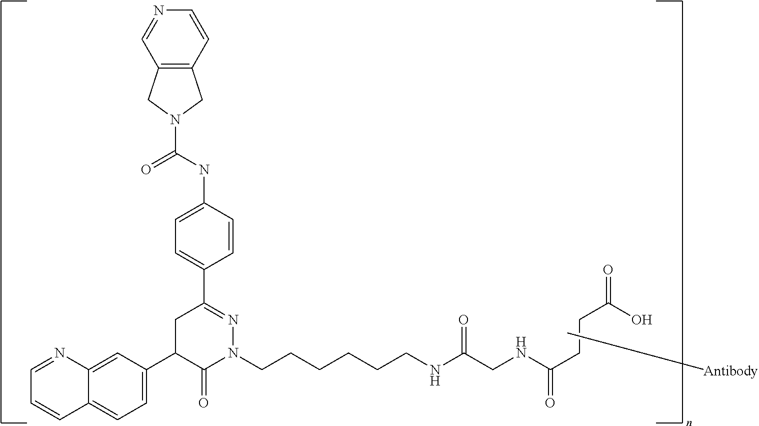

5 mg of anti-HER2 TPP-1015 (c=12.2 mg/mL) were coupled with N-{4-[1-{6-[2-(2,5-dioxo-2,5-dihydro-1H-pyrrol-1-yl)acetamido]hexyl}-6-oxo-5-(quinolin-7-yl)-1,4,5,6-tetrahydropyridazin-3-yl]phenyl}-1,3-dihydro-2H-pyrrolo[3,4-c]pyridine-2-carboxamide (see Final intermediate 46-1, 370 μg, 0.53 μmol) according procedure 2 and the reaction was, after Sephadex purification, concentrated by ultracentrifugation and rediluted with PBS.

Protein concentration: 1.92 mg/mL

Drug/mAb ratio: 7.2 (UV)

### Intermediate 47-1

### Tert-Butyl N²-(tert-butoxycarbonyl)-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparaginate

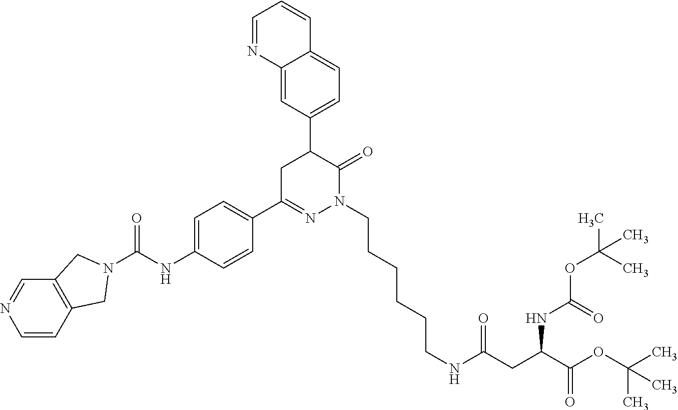 [SMILES: CC(C)(C)OC(=O)N[C@H](CC(=O)NCCCCCCN1N=C(c2ccc(NC(=O)N3Cc4ccncc4C3)cc2)CC(c2ccc3cccnc3c2)C1=O)C(=O)OC(C)(C)C]

4-Methylmorpholine (55 μl, 500 μmol) was added at r.t. to a mixture of (3R)-4-tert-butoxy-3-[(tert-butoxycarbonyl)amino]-4-oxobutanoic acid (36.0 mg, 124 μmol) in DMF (1.9 mL) and the mixture stirred for 30 min at that temperature. After that HATU (56.8 mg, 149 μmol) was added and the mixture stirred for further 30 min at r.t. after which N-{4-[1-(6-aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide hydrogen chloride (see Example 45, 79.0 mg, 124 μmol) was added. After stirring for 14 h at r.t. the mixture was diluted with dichloromethane/isopropanol and washed with water. The organic phase was washed with brine and filtered through a phase separator filter and concentrated under reduced pressure. The crude product was purified by preparative HPLC to give the title compound (67.0 mg, 65% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18 10 μm 120×30 mm. Eluent A: water+0.1 Vol-% formic acid; Eluent B: acetonitrile; gradient: 0-20 min 15-55% B. rate 150 mL/min, temperature 25° C.

LC-MS (Method 1): Rₜ=1.04 min; MS (ESIpos): m/z=834 [M+H]⁺.

### Intermediate 47-2

### N-{6-[3-{4-[(1,3-Dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparagine

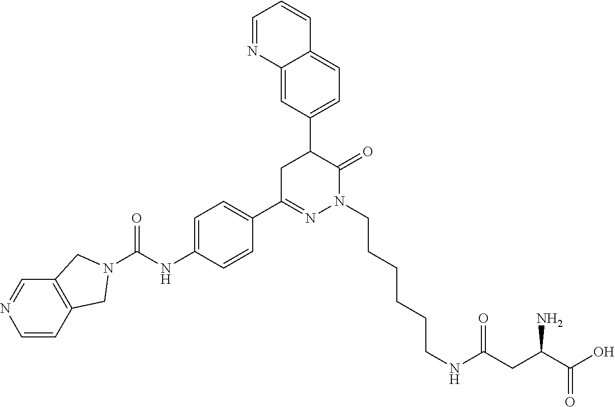 [SMILES: N[C@H](CC(=O)NCCCCCCN1N=C(c2ccc(NC(=O)N3Cc4ccncc4C3)cc2)CC(c2ccc3cccnc3c2)C1=O)C(=O)O]

A mixture of tert-butyl N²-(tert-butoxycarbonyl)-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparaginate (67.0 mg, 80.4 μmol), TFA (93 μl, 1.2 mmol) and dichloromethane (780 μL) was stirred at r.t. for 14 h. After that the mixture was concentrated under reduced pressure and coevaporated twice with toluene to give the title compound (40.0 mg, 73% yield).

### Final Intermediate 47-3

### N-{6-[3-{4-[(1,3-Dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-N²-[(2,5-dioxo-2,5-dihydro-1H-pyrrol-1-yl)acetyl]-D-asparagine

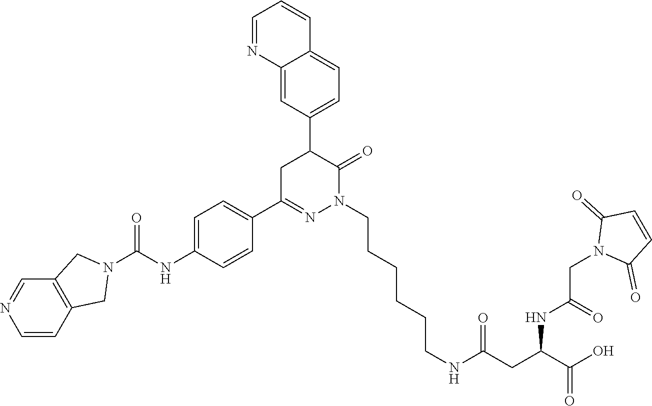 [SMILES: O=C(C[C@@H](NC(=O)CN1C(=O)C=CC1=O)C(=O)O)NCCCCCCN1N=C(c2ccc(NC(=O)N3Cc4ccncc4C3)cc2)CC(c2ccc3cccnc3c2)C1=O]

A mixture of N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparagine (40.0 mg, 59.1 μmol), maleimidoacetic acid N-hydroxysuccinimide ester (14.9 mg, 59.1 μmol), DIPEA (31 μl, 180 μmol) and DMF (680 μL) was stirred for 14 h at r.t. After that the mixture was filtered and purified by preparative HPLC to give the title compound (11.0 mg, 23% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18 10 μM 120×20 mm. Eluent A: water; Eluent B: acetonitrile; gradient: 0-22 min 10-50% B. rate 50 mL/min, temperature 25° C.

LC-MS (Method 1): Rₜ=0.73 min; MS (ESpos): m/z=814 [M+H]⁺.

¹H-NMR (400 MHz, DMSO-d6) δ [ppm]: 1.228 (0.80), 1.304 (2.40), 1.349 (0.80), 1.369 (0.80), 1.683 (0.80), 2.518 (16.00), 2.523 (10.00), 2.539 (8.40), 2.674 (2.00), 2.727 (0.40), 2.886 (0.40), 3.002 (1.20), 3.017 (1.20), 3.793 (0.40), 3.810 (0.80), 3.829 (0.80), 3.845 (0.40), 4.043 (2.80), 4.048 (2.80), 4.143 (0.40), 4.163 (0.80), 4.182 (0.40), 4.477 (0.40), 4.803 (2.40), 4.821 (2.40), 7.064 (8.40), 7.426 (1.20), 7.438 (1.20), 7.487 (1.20), 7.497 (1.20), 7.508 (1.20), 7.518 (1.20), 7.551 (0.80), 7.555 (1.20), 7.572 (1.20), 7.577 (1.20), 7.635 (2.00), 7.658 (2.80), 7.742 (3.20), 7.764 (2.00), 7.841 (2.00), 7.939 (2.00), 7.960 (1.60), 8.319 (1.20), 8.337 (0.80), 8.429 (0.40), 8.489 (1.60), 8.501 (1.60), 8.603 (2.40), 8.647 (1.60), 8.855 (1.20), 8.859 (1.20), 8.865 (1.20), 8.870 (1.20).

### Example 47A

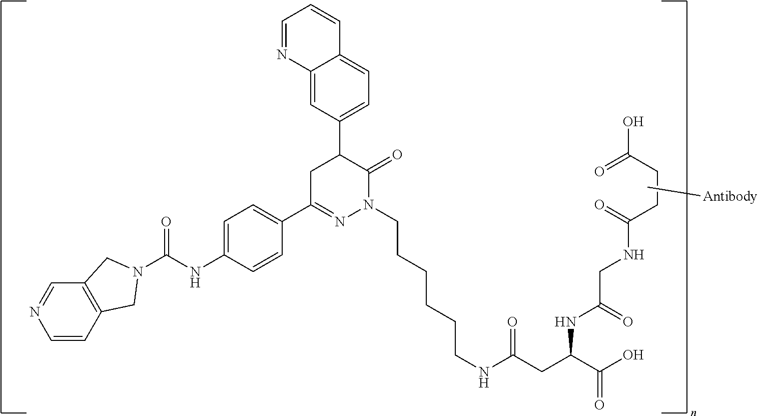

5 mg of anti-HER2 TPP-1015 (c=12.2 mg/mL) were coupled with N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-N²-[(2,5-dioxo-2,5-dihydro-1H-pyrrol-1-yl)acetyl]-D-asparagine (see Final Intermediate 47-3, 430 μg, 0.53 μmol) according procedure 2 and the reaction was, after Sephadex purification, concentrated by ultracentrifugation and rediluted with PBS.

Protein concentration: 2.11 mg/mL

Drug/mAb ratio: 6.2 (UV)

### Example 47D

5 mg of anti-C4.4a TPP-509 (c=11.88 mg/mL) were coupled with N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-N²-[(2,5-dioxo-2,5-dihydro-1H-pyrrol-1-yl)acetyl]-D-asparagine (see Final Intermediate 47-3, 430 μg, 0.53 μmol) according procedure 2 and the reaction was, after Sephadex purification, concentrated by ultracentrifugation and rediluted with PBS.

Protein concentration: 0.21 mg/mL

Drug/mAb ratio: 6.2 (UV)

### Intermediate 48-1

### Tert-Butyl [(2S)-1-({6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}amino)-1-oxopropan-2-yl]carbamate

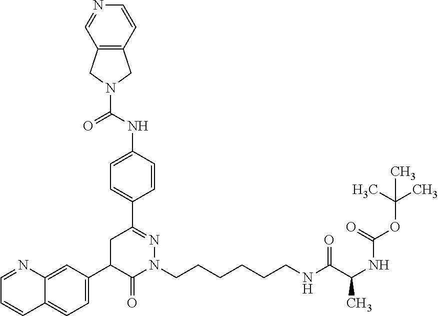 [SMILES: C[C@H](NC(=O)OC(C)(C)C)C(=O)NCCCCCCN1N=C(c2ccc(NC(=O)N3Cc4ccncc4C3)cc2)CC(c2ccc3cccnc3c2)C1=O]

To a solution of N-(tert-butoxycarbonyl)-L-alanine (22.7 mg, 120 μmol) in DMF (1.8 mL) was added at r.t. 4-methylmorpholine (53 μl, 480 μmol) and HATU (47.8 mg, 126 μmol) and the mixture was stirred for 30 min at that temperature. Then N-{4-[1-(6-aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide-hydrogen chloride (Example 45, 76.0 mg, 120 μmol) was added and the mixture stirred for further 14 h. After that the mixture was taken up in dichloromethane/methanol/water, the layers were separated and the organic phase was filtered through a silicone filter and concentrated under reduced pressure to give the title compound (100 mg, 90% purity, quant.).

LC-MS (Method 1): Rₜ=0.89 min; MS (ESIpos): m/z=733 [M+H]⁺.

### Intermediate 48-2

### N-{4-[1-[6-(L-Alanylamino)hexyl]-6-oxo-5-(quinolin-7-yl)-1,4,5,6-tetrahydropyridazin-3-yl]phenyl}-1,3-dihydro-2H-pyrrolo[3,4-c]pyridine-2-carboxamide

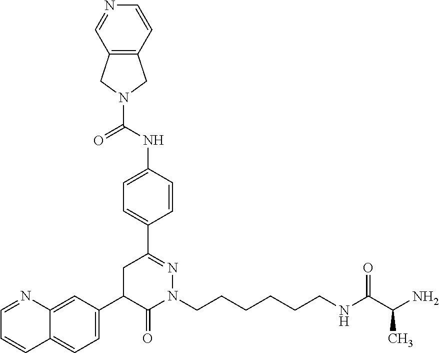 [SMILES: C[C@H](N)C(=O)NCCCCCCN1N=C(c2ccc(NC(=O)N3Cc4ccncc4C3)cc2)CC(c2ccc3cccnc3c2)C1=O]

A mixture of tert-butyl [(2S)-1-({6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}amino)-1-oxopropan-2-yl]carbamate (100 mg, 136 μmol), trifluoroacetic acid (160 μl, 2.0 mmol), and dichloromethane (1.8 mL) was stirred at r.t. for 14 h. After that the mixture was concentrated under reduced pressure and coevaporated twice with toluene. The crude product was purified by preparative HPLC to give the title compound (30.0 mg, 34% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: xBridge C18, 5 μM, 100×30 mm. Eluent A: water+0.1 Vol-% ammonia; Eluent B: acetonitrile; gradient: 0-20 min 15-55% B. rate 60 mL/min, temperature 25° C.

LC-MS (Method 1): Rₜ=0.66 min, MS (ESipos): m/z=633 [M+H]⁺.

¹H-NMR (400 MHz, DMSO-d₆) δ [ppm]: 1.101 (6.72), 1.118 (6.87), 1.231 (0.83), 1.302 (2.64), 1.353 (0.68), 1.370 (0.91), 1.386 (1.13), 1.679 (1.06), 2.331 (3.17), 2.336 (1.43), 2.518 (16.00), 2.523 (10.34), 2.673 (3.17), 2.678 (1.43), 3.010 (0.53), 3.026 (1.21), 3.046 (1.21), 3.228 (0.45), 3.245 (1.28), 3.263 (1.28), 3.280 (0.68), 3.385 (1.21), 3.402 (1.06), 3.412 (0.98), 3.435 (0.98), 3.789 (0.53), 3.806 (0.91), 3.830 (0.91), 3.847 (0.53), 4.149 (0.68), 4.169 (1.06), 4.188 (0.60), 4.806 (2.42), 4.824 (2.42), 7.427 (1.36), 7.440 (1.43), 7.490 (1.66), 7.501 (1.66), 7.511 (1.66), 7.522 (1.58), 7.557 (1.21), 7.562 (1.28), 7.579 (1.28), 7.583 (1.28), 7.646 (2.64), 7.668 (3.77), 7.750 (3.92), 7.772 (2.49), 7.797 (0.83), 7.847 (2.11), 7.942 (2.19), 7.963 (1.96), 8.322 (1.13), 8.339 (1.58), 8.493 (2.11), 8.506 (1.96), 8.608 (2.79), 8.642 (2.42), 8.860 (1.66), 8.865 (1.74), 8.871 (1.66), 8.876 (1.51).

### Intermediate 48-3

### N-(tert-Butoxycarbonyl)-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide

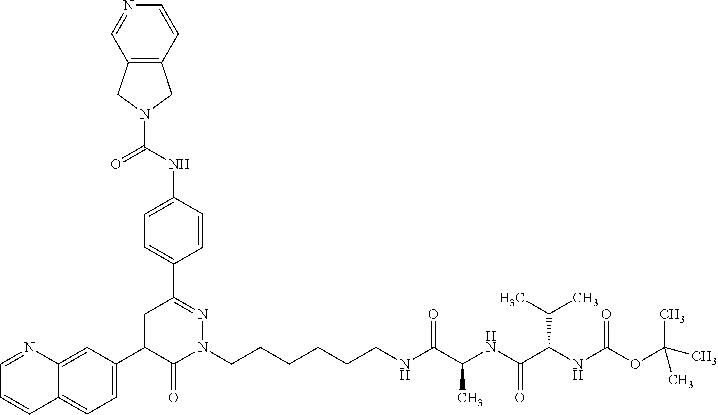 [SMILES: CC(C)[C@H](NC(=O)OC(C)(C)C)C(=O)N[C@@H](C)C(=O)NCCCCCCN1N=C(c2ccc(NC(=O)N3Cc4ccncc4C3)cc2)CC(c2ccc3cccnc3c2)C1=O]

To a solution of N-(tert-butoxycarbonyl)-L-valine (11.3 mg, 52.2 μmol) in DMF (730 μL) was added at r.t. 4-methylmorpholine (18 μl, 170 μmol) and HATU (19.8 mg, 52.2 μmol) and the mixture was stirred for 30 min at that temperature. Then N-{4-[1-[6-(L-alanylamino)hexyl]-6-oxo-5-(quinolin-7-yl)-1,4,5,6-tetrahydropyridazin-3-yl]phenyl}-1,3-dihydro-2H-pyrrolo[3,4-c]pyridine-2-carboxamide (30.0 mg, 47.4 μmol) was added and the mixture stirred for further 14 h. After that the mixture was filtered and purified by preparative HPLC to give the title compound (7.0 mg, 18% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18, 10 μM, 120×30 mm. Eluent A: water+0.1 Vol-% formic acid; Eluent B: acetonitrile; gradient: 0-20 min 15-55% B. rate 150 mL/min, temperature 25° C.

LC-MS (Method 1): Rₜ=0.96 min; MS (ESIpos): m/z=832 [M+H]⁺.

### Intermediate 48-4

### L-Valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide

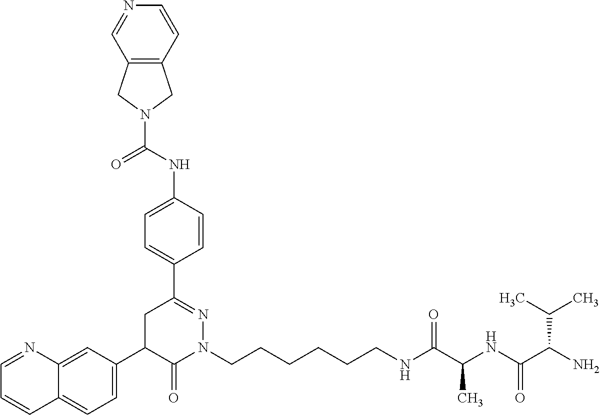 [SMILES: CC(C)[C@H](N)C(=O)N[C@@H](C)C(=O)NCCCCCCN1N=C(c2ccc(NC(=O)N3Cc4ccncc4C3)cc2)CC(c2ccc3cccnc3c2)C1=O]

A mixture of N-(tert-butoxycarbonyl)-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide (7.00 mg, 8.41 μmol), trifluoroacetic acid (9.7 μl, 130 μmol), and dichloromethane (160 μL) was stirred at r.t. for 4 h. After that the mixture was concentrated under reduced pressure and coevaporated twice with toluene to give the crude product which was directly used in the next step.

LC-MS (Method 1): Rₜ=0.71 min; MS (ESIpos): m/z=732 [M+H]⁺.

### Final Intermediate 48-5

### N-[(2,5-dioxo-2,5-dihydro-1H-pyrrol-1-yl)acetyl]-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide

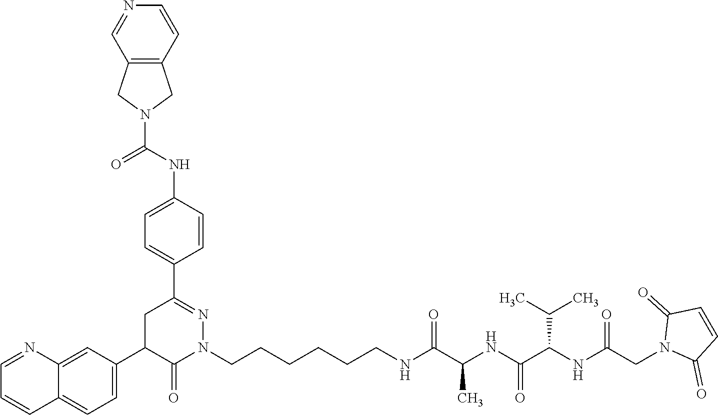 [SMILES: CC(C)[C@H](NC(=O)CN1C(=O)C=CC1=O)C(=O)N[C@@H](C)C(=O)NCCCCCCN1N=C(c2ccc(NC(=O)N3Cc4ccncc4C3)cc2)CC(c2ccc3cccnc3c2)C1=O]

To a solution of L-valyl-N-{6-[3-{4-[(1,3-dihydro-2H-pyrrolo[3,4-c]pyridine-2-carbonyl)amino]-phenyl}-6-oxo-5-(quinolin-7-yl)-5,6-dihydropyridazin-1(4H)-yl]hexyl}-L-alaninamide (6.66 mg, 9.09 μmol) in DMF (140 μL) was added at r.t. DIPEA (6.3 μl, 36 μmol) and 1-{2-[(2,5-dioxopyrrolidin-1-yl)oxy]-2-oxoethyl}-1H-pyrrole-2,5-dione (2.52 mg, 10.0 μmol) and the mixture stirred for 14 h at that temperature. After that more 1-{2-[(2,5-dioxopyrrolidin-1-yl)oxy]-2-oxoethyl}-1H-pyrrole-2,5-dione (2.29 mg, 9.09 μmol) and DIPEA (27 μmol) were added and the mixture stirred for further 3 h. Then the mixture was filtered and purified by preparative HPLC to give the title compound (6.0 mg, 76% yield).

HPLC: Instrument: Labomatic HD-3000, pump head HDK-280, gradient module NDB-1000, fraction collector Labomatic Labocol Vario 4000, Knauer UV detector Azura UVD 2.15, Prepcon 5 software. Column: Chromatorex C18, 10 μM, 120×20 mm. Eluent A: water+0.1 Vol-% formic acid; Eluent B: acetonitrile; gradient: 0-20 min 15-55% B. rate 50 mL/min, temperature 25° C.

LC-MS (Method 1): Rₜ=0.80 min; MS (ESIpos): m/z=870 [M+H]⁺.

¹H-NMR (400 MHz, DMSO-d₆) δ [ppm]: 0.784 (1.83), 0.801 (1.89), 0.814 (1.89), 0.831 (1.89), 1.169 (2.02), 1.187 (2.02), 1.233 (0.43), 1.287 (1.10), 1.352 (0.61), 1.667 (0.43), 1.907 (0.43), 2.332 (2.50), 2.336 (1.16), 2.518 (16.00), 2.522 (10.32), 2.539 (0.73), 2.590 (0.61), 2.673 (2.63), 2.678 (1.16), 3.012 (0.49), 3.305 (0.55), 3.383 (0.43), 4.102 (1.95), 4.153 (0.49), 4.169 (0.79), 4.192 (0.55), 4.213 (0.55), 4.806 (0.98), 4.825 (0.98), 7.077 (6.47), 7.425 (0.55), 7.439 (0.55), 7.488 (0.73), 7.498 (0.67), 7.508 (0.67), 7.519 (0.73), 7.554 (0.49), 7.558 (0.55), 7.575 (0.49), 7.580 (0.55), 7.646 (1.04), 7.668 (1.47), 7.716 (0.55), 7.749 (1.53), 7.771 (0.98), 7.852 (0.85), 7.939 (0.92), 7.961 (0.79), 8.233 (0.49), 8.256 (0.49), 8.320 (0.49), 8.341 (0.49), 8.492 (0.79), 8.504 (0.79), 8.606 (1.10), 8.638 (0.98), 8.859 (0.67), 8.863 (0.73), 8.870 (0.73), 8.874 (0.67).

### Example 48A

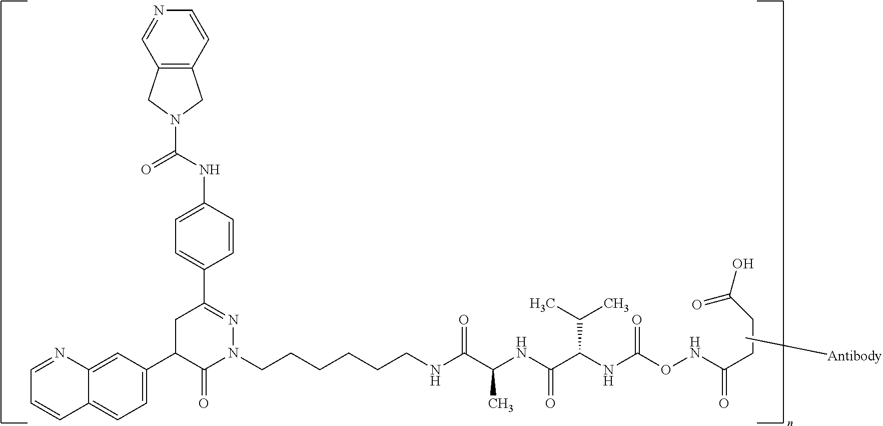

5 mg of anti-HER2 TPP-1015 (c=12.2 mg/mL) were coupled with N-[(2,5-dioxo-2,5-dihydro-1H-pyrrol-1-yl)acetyl]-L-valyl-N-{6-[3-{4-[(1,3-dihydro-2H-pyrrolo[3,4-c]pyridine-2-carbonyl)-amino]phenyl}-6-oxo-5-(quinolin-7-yl)-5,6-dihydropyridazin-1(4H)-yl]hexyl}-L-alaninamide (see Final Intermediate 48-5, 460 μg, 0.53 μmol) according procedure 2 and the reaction was, after Sephadex purification, concentrated by ultracentrifugation and rediluted with PBS.

Protein concentration: 0.92 mg/mL

Drug/mAb ratio: 1.4 (UV)

### Example 48C

5 mg of anti-B7H3 TPP-8382 (c=15.1 mg/mL) were coupled with N-[(2,5-dioxo-2,5-dihydro-1H-pyrrol-1-yl)acetyl]-L-valyl-N-{6-[3-{4-[(1,3-dihydro-2H-pyrrolo[3,4-c]pyridine-2-carbonyl)-amino]phenyl}-6-oxo-5-(quinolin-7-yl)-5,6-dihydropyridazin-1(4H)-yl]hexyl}-L-alaninamide (see Final Intermediate 48-5, 460 μg, 0.53 μmol) according procedure 2 and the reaction was, after Sephadex purification, concentrated by ultracentrifugation and rediluted with PBS.

Protein concentration: 0.95 mg/mL

Drug/mAb ratio: 1.6 (UV)

### Example 48D

5 mg of anti-C4.4a TPP-509 (c=11.88 mg/mL) were coupled with N-[(2,5-dioxo-2,5-dihydro-1H-pyrrol-1-yl)acetyl]-L-valyl-N-{6-[3-{4-[(1,3-dihydro-2H-pyrrolo[3,4-c]pyridine-2-carbonyl)-amino]phenyl}-6-oxo-5-(quinolin-7-yl)-5,6-dihydropyridazin-1(4H)-yl]hexyl}-L-alaninamide (see Final Intermediate 48-5, 460 μg, 0.53 μmol) according procedure 2 and the reaction was, after Sephadex purification, concentrated by ultracentrifugation and rediluted with PBS.

Protein concentration: 0.34 mg/mL

Drug/mAb ratio: 0.8 (UV)

### Cell Proliferation Assay for Nampt-ADCs and Nampt-SMOLs

### Cell Titer Glo (CTG) Method

Cells were plated in 75 μL growth medium per well in a 96-well plate (white/clear bottom, (#¹⁰⁷⁷⁵⁵⁸⁴, Perkin Elmer) at the indicated cell number (Table 1). The plates were incubated overnight at 37° C. 24 h after seeding of cells, test compounds were serially diluted in growth medium, and 25 μL of four fold concentrated dilutions/well were added to the test plates. For antibody-drug-conjugates, typically semi-logarithmic dilutions from 300 nM to 0.03 nM in triplicates were used. The compound treated plates were incubated for 72 or 96 hrs at 37° C. as indicated. In parallel to the addition of test compounds, time zero Cell Titer Glo Luminescent Cell Viability Assay (CTG) levels were measured in sister plates. To this end, 75 μL per well CTG solution (Promega, catalog #G755B and G756B) was added to cells in sister plates, incubated for 10 min, and luminescence was measured on a VICTOR V instrument (Perkin Elmer). After incubation for either 72 or 96 hrs (Table 1) in the presence of test compounds, 100 μl per well CTG solution was added to all test wells, incubated for 10 min and luminescence was measured on a VICTOR V instrument. Dose response curves and calculation of IC50 values (50% inhibition of proliferation) were generated using BELLA-Dose Response Curve (DRC) spreadsheets. The DRC software is a Biobook Spreadsheet that was developed by Bayer AG and Bayer Business Services on the IDBS E-Workbook Suite platform (IDBS: ID Business Solutions Ltd., Guildford, UK).

TABLE 1
Assay conditions for cell lines in cell proliferation assay

| Cancer | Source | | | | |
|---|---|---|---|---|---|
| Cell | Catalogue | | No of | Growth | Endpoint |
| Line | # | Indication | cells/well | medium | after |
| THP-1 | ATCC | AML | 4000 | RPMI 1640 (Biochrom, | 72 h |
| | #TIB-202 | | | #FG1215) + 10% FCS | |
| | | | | Biochrom, #S0615) + | |
| | | | | 0.05 mM β-Mercapto- | |
| | | | | ethanol (Sigma, | |
| | | | | #M6250) + 1% Pen-Strep | |
| | | | | (Biochrom, #A2212) | |
| U251 MG | ECACC | Glioblastoma | 2000 | RPMI 1640 + 10% FCS + | 72 h |
| (formerly | #09063001 | | | 1% Pen-Strep | |
| known as | | | | | |
| U-373 | | | | | |
| MG) | | | | | |
| MDA- | DSMZ | Breast | 2500 | RPMI1640 (Biochrom, | 96 h |
| MB-453 | #ACC 65 | cancer | | #FG1215 with stable | |
| | | | | glutamine, 20% FCS | |
| | | | | (Sigma, #0626D) + 1% | |
| | | | | Pen-Strep | |
| MDA- | ATCC | Breast | 2500 | DMEM/Ham′s F12 | 96 |
| MB-453 | #HTB-131 | cancer | | (Biochrom #FG4815 with | |
| | | | | stable glutamine) + 20% | |
| | | | | FCS 1% Pen-Strep | |
| REC-1 | ATCC | Mantle cell | 3000 | RPMI1640 (Biochrom, | 72 h |
| | #CRL-3004 | lymphoma | | #FG1215 with stable | |
| | | | | glutamine) + 10% FCS | |
| | | | | (Sigma, #0626D) + 1% | |
| | | | | Pen-Strep | |

NAMPT Biochemical Assay (hNAMPT IC50)

Nicotinamide phosphoribosyltransferase (NAMPT) inhibitory activity of compounds of the present invention was quantified employing a cascade assay as described in the following paragraphs. The assay couples the conversion of nicotinamide (NAM) to nicotinamide mononucleotide (NMN) by NAMPT with the conversion of NMN to nicotine adenine dinucleotide (NAD⁺) by nicotinamide mononucleotide adenylyltransferase 1 (NMNAT1) and the subsequent quantification of the generated NAD⁺ by a commercial detection kit (NAD/NADH-Glo™ Assay from Promega, #G9072).

N-terminally His₆-tagged recombinant full length human NAMPT and N-terminally His₆-tagged recombinant full length human NMNAT1, both expressed in E. coli and purified via Ni-NTA-affinity chromatography and consecutive size exclusion chromatography, were used as enzymes.

For the assay 50 nl of a 100 fold concentrated solution of the test compound in DMSO was pipetted into a white low volume 384 well microtiter plate (Greiner Bio-One, Frickenhausen, Germany), 2.5 μl of a solution of NAMPT in aqueous NMNAT1-containing assay buffer [50 mM Tris/HCl pH 7.5, 12 mM MgCl₂, 0.6 mM adenosine-tri-phosphate (ATP), 1 nM NMNAT1, 0.02% (w/v) bovine serum albumin (Sigma-Aldrich #P7906), 0.001% (v/v) Tween-20 (Sigma-Aldrich #P7949)] were added and the mixture was incubated for 15 min at 22° C. to allow pre-binding of the test compounds to the enzyme before the start of the enzyme reaction. Then the reaction was started by the addition of 2.5 μl of a solution of NAM (300 nM=>final conc. in the 5 μl assay volume is 150 nM, Sigma-Aldrich #⁴⁷⁸⁶⁵) and 5-phosphorylribose-1-pyrophosphate pentasodium salt (PRPP, 1.2 μM=>final conc. in the 5 μl assay volume is 0.6 μM, Sigma-Aldrich P8296) in assay buffer and the resulting mixture was incubated for a reaction time of 20 min at 22° C. The concentration of NAMPT was adjusted depending of the activity of the enzyme lot and was chosen appropriate to have the assay in the linear range, typical final concentration in the 5 μl assay volume was 0.16 nM. The NAM conversion was stopped and the detection of the generated NAD⁺ started by the addition of 2.5 μl of a solution of 600 nM FK866, a NAMPT inhibitor commercially available (e.g. from Selleckchem), in detection reagent solution (1:4.5 fold dilution of NAD/NADH-Go™ Detection Reagent [Promega] in water).

The resulting mixture was incubated 2 h at 22° C. to allow a steady-state of the detection system. Subsequently the generated luminescence was measured in a suitable luminescence reader, e.g. a Viewlux™ (Perkin-Elmer), and taken as a measure for the generated NAD⁺. The data were normalised (enzyme reaction without inhibitor=0% inhibition, all other assay components but no NAMPT=100% inhibition). Usually the test compounds were tested on the same microtiter plate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values were calculated by a 4-parameter-fit.

Table 2 below lists the IC₅₀ values of representative examples and metabolites

| | NAMPT | | | | |
|---|---|---|---|---|---|
| | biochemical | MDA-MB-453 | A549 C4.4a B4 | THP-1 | U251 |
| | assay | 72 h | 72 h | 72 h | 72 h |
| Example | IC50 | IC50 | IC50 | IC50 | IC50 |
| No | [mol/l] | [mol/l] | [mol/l] | [mol/l] | [mol/1] |
| 01 | 1.06E−9 | <3.00E−11 | 9.35E−10 | <3.00E−12 | <3.00E−11 |
| | 1.21E−9 | 4.16E−11 | 3.13E−9 | | 1.26E−10 |
| | 1.26E−9 | 4.64E−11 | 6.03E−9 | | |
| | 1.39E−9 | 6.13E−11 | 6.86E−9 | | |
| | | 7.23E−11 | 8.40E−9 | | |
| | | 8.30E−11 | 8.81E−9 | | |
| | | 8.40E−11 | 8.83E−9 | | |
| | | 1.55E−10 | 9.39E−9 | | |
| | | | 1.36E−8 | | |
| | | | 1.58E−8 | | |
| | | | 2.89E−8 | | |
| | | | 3.04E−8 | | |
| 02 | 3.50E−10 | | | | |
| | 3.65E−10 | | | | |
| 03 | 2.07E−10 | | | | |
| | 2.14E−10 | | | | |
| 04 | 1.16E−9 | <3.00E−11 | >3.00E−7 | 1.71E−11 | 1.46E−10 |
| | 1.22E−9 | 8.10E−10 | | | |
| 05 | 1.47E−10 | | | | |
| | 1.76E−10 | | | | |
| 06 | 2.67E−10 | <3.00E−11 | 1.10E−8 | <3.00E−12 | <3.00E−11 |
| | 2.88E−10 | | | | |
| 07 | 1.02E−9 | 2.99E−10 | >3.00E−7 | 9.95E−10 | 8.95E−9 |
| | 1.20E−9 | | | | |
| 08 | 1.04E−9 | 2.66E−9 | 9.35E−8 | 9.79E−10 | 2.81E−8 |
| | 1.32E−9 | | | | |
| 09 | 4.57E−10 | 3.57E−11 | 9.72E−8 | 1.21E−9 | 4.34E−9 |
| | 7.17E−10 | | | | |
| 10 | 3.58E−10 | 9.43E−11 | 2.67E−7 | 4.71E−12 | 8.87E−9 |
| | 3.89E−10 | | | | |
| 11 | 3.18E−10 | <3.00E−11 | >3.00E−7 | <3.00E−12 | 2.82E−9 |
| | 3.79E−10 | 1.71E−10 | | | |
| | | 2.70E−10 | | | |
| 12 | 5.39E−10 | <3.00E−12 | 4.72E−10 | <3.00E−12 | <3.00E−11 |
| | 5.51E−10 | <3.00E−11 | | | |
| 13 | 2.77E−9 | 4.36E−10 | 2.27E−7 | 4.47E−10 | 4.37E−8 |
| | 3.20E−9 | | | | |
| 14 | 3.17E−10 | | | | |
| | 3.87E−10 | | | | |
| 15 | 3.09E−10 | <3.00E−11 | 8.87E−9 | | |
| | 3.63E−10 | | 1.38E−8 | | |
| | | | 2.45E−8 | | |
| 16 | 3.23E−10 | <3.00E−11 | 2.32E−8 | <3.00E−12 | 4.11E−11 |
| | 3.26E−10 | | | | 5.70E−11 |
| 17 | 2.18E−10 | <3.00E−11 | 1.24E−8 | 3.85E−11 | 6.42E−10 |
| | 3.46E−10 | | | | |
| 18 | 2.13E−10 | <3.00E−11 | 5.85E−8 | 3.00E−12 | <3.00E−11 |
| | 2.26E−10 | | | | 4.28E−11 |
| 20 | 1.56E−10 | <3.00E−11 | 6.39E−9 | <3.00E−12 | <3.00E−11 |
| | 2.29E−10 | | | | |
| 21 | 3.93E−10 | <3.00E−11 | 4.05E−8 | <3.00E−12 | <3.00E−11 |
| | 4.70E−10 | | | | |
| 22 | 1.11E−10 | <3.00E−11 | 4.88E−9 | | |
| | 1.44E−10 | | | | |
| 24 | 2.41E−10 | <3.00E−11 | 4.92E−9 | 9.96E−12 | <3.00E−11 |
| | 2.95E−10 | | | | |
| 25 | 8.03E−10 | <3.00E−11 | 5.71E−8 | 2.27E−11 | 4.28E−10 |
| | 1.15E−9 | | | | |
| 26 | 1.67E−10 | <3.00E−11 | 1.11E−7 | | |
| | 2.07E−10 | | | | |
| 29 M | 4.41E−10 | | | | |
| | 6.37E−10 | | | | |
| 31 M | 2.60E−10 | | | | |
| | 3.11E−10 | | | | |
| 34 M | 1.17E−9 | 9.12E−8 | >3.00E−7 | >3.00E−7 | >3.00E−7 |
| | 1.18E−9 | | | | |
| 36 M | 1.03E−9 | 1.80E−8 | 2.99E−7 | | |
| | 1.05E−9 | | | | |
| 45 | 2.23E−10 | <3.00E−11 | 3.20E−8 | | |
| | 2.41E−10 | 5.40E−10 | 4.97E−8 | | |
| | 2.77E−10 | | | | |
| | 3.43E−10 | | | | |

Table 3 below lists the IC₅₀ values of representative ADCs proliferation assays

| | MDA- | | | | |
|---|---|---|---|---|---|
| | MB-453 | A549 | | | |
| | DSMZ | C4.4a | THP-1 | U-251 | Rec-1 |
| | 72 h | 72 h | 72 h | 72 h | 72 h |
| Example | IC⁵⁰ | IC⁵⁰ | IC⁵⁰ | IC⁵⁰ | IC50 |
| No | [mol/l] | [mol/l] | [mol/l] | [mol/l] | [mol/l] |
| 27 C | | | 5.85E−9 | 1.04E−8 | |
| | | | 1.04E−8 | 2.00E−8 | |
| 27 C | | | 1.46E−9 | 7.43E−9 | |
| | | | | 1.42E−8 | |
| 28 D | 1.50E−8 | 9.93E−9 | | | |
| 29 C | | | 3.09E−9 | 7.90E−8 | |
| 29 D | 8.79E−10 | 1.43E−7 | | | |
| 30 Aa | 5.64E−11 | >3.00E−7 | | | |
| 30 Ab | <3.00E−11 | >3.00E−7 | | | |
| 30 Ca | | | 1.70E−11 | 1.45E−8 | |
| | | | 2.11E−11 | | |
| 30 Cb | | | <3.00E−11 | 6.51E−10 | |
| 30 D | 3.15E−8 | 5.15E−9 | | | |
| 30 F | 8.66E−9 | 5.10E−9 | | | |
| 30 M | 9.96E−9 | >3.00E−7 | 8.11E−8 | >3.00E−7 | |
| 31 C | | | 2.08E−11 | 8.30E−9 | |
| | | | 3.10E−11 | | |
| 31 D | 1.14E−8 | >3.00E−7 | | | |
| 31 E | 5.53E−9 | >3.00E−7 | | | |
| 32 C | | | 4.29E−10 | <3.00E−11 | |
| | | | 9.70E−10 | | |
| 32 E | 1.39E−9 | >3.00E−7 | | | |
| 33 C | | | 1.40E−11 | 2.67E−8 | |
| 34 Ca | | | 3.42E−11 | 2.88E−8 | |
| 34 Cb | | | <3.00E−11 | 1.43E−8 | |
| 34 Da | 1.90E−8 | >3.00E−7 | | | |
| 34 Ea | 9.08E−9 | >3.00E−7 | | | |
| 34 Eb | 2.23E−9 | >3.00E−7 | | | |
| 34 Db | 2.32E−9 | >3.00E−7 | | | |
| 35 A | 3.23E−11 | >3.00E−7 | | | |
| 35 C | | | 1.75E−10 | 3.11E−9 | |
| 35 D | 1.68E−9 | >3.00E−7 | | | |
| 36 A | 4.40E−11 | >3.00E−7 | | | |
| 36 Ca | | | 8.77E−11 | 9.27E−9 | |
| 36 Cb | | | <3.00E−11 | 1.93E−9 | |
| 36 Da | 1.94E−8 | 3.60E−9 | | | |
| 36 Db | 7.24E−9 | 3.13E−9 | | | |
| 36 Ea | 4.88E−9 | 3.59E−9 | | | |
| 36 Eb | 3.78E−10 | 2.38E−9 | | | |
| 37 A | 5.53E−11 | 1.69E−8 | | | |
| 37 C | | | 3.84E−11 | 1.68E−10 | |
| 37 D | 5.96E−10 | 7.50E−8 | | | |
| 37 E | 3.19E−10 | 3.23E−8 | | | |
| 38 A | 5.72E−11 | 3.08E−8 | | | |
| 38 B | | | | | 2.43E−9 |
| 38 C | | | 5.03E−11 | 2.46E−10 | |
| 39 A | 4.49E−10 | 2.11E−7 | | | |
| 39 C | | | 1.12E−9 | 9.84E−9 | |
| 40 A | 8.24E−11 | >3.00E−7 | | | |
| 40 B | | | | | 9.03E−10 |
| 40 D | 1.36E−9 | 1.97E−7 | | | |
| 40 E | 1.17E−9 | 2.04E−7 | | | |
| 41 A | <3.00E−11 | >3.00E−7 | | | |
| | 4.36E−11 | | | | |
| | 5.64E−11 | | | | |
| 42 A | <3.00E−11 | >3.00E−7 | | | |
| | 3.76E−11 | | | | |
| | 4.28E−11 | | | | |
| 42 E | 1.87E−9 | 6.35E−9 | | | |
| | 4.43E−9 | | | | |
| | 4.87E−9 | | | | |
| 43 D | 3.12E−9 | >3.00E−7 | | | |
| 43 E | 6.42E−9 | >3.00E−7 | | | |
| 44 A | 1.48E−10 | >3.00E−7 | | | |
| 46 A | 4.09E−11 | >3.00E−7 | | | |
| 47 A | 5.15E−11 | >3.00E−7 | | | |
| 47 D | 2.17E−8 | 9.93E−9 | | | |
| 48 A | 5.01E−10 | >3.00E−7 | | | |
| | 5.79E−10 | | | | |
| 48 D | 7.28E−8 | >3.00E−7 | | | |
| | >3.00E−7 | | | | |

1: A conjugate of a binder or a derivative thereof with one or more molecules of an active compound that has the formula: 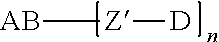
wherein AB stands for a binder, Z′ stands for a linker, n stands for a number between 1 and 50, and
D stands for an active component of Formula (I-D): 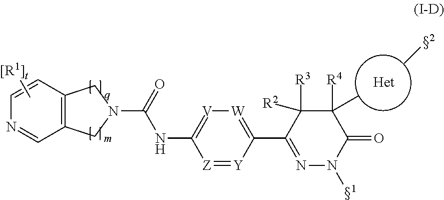
wherein:
§¹ or §² represent the point of attachment to linker Z′, with the proviso that:
when linker Z′ is connected at §¹, then §² represents R⁵, and
when linker Z′ is connected at §², then linker Z′ is connected to a carbon or nitrogen atom of ring Het and §¹ represents R^{5b};
Het represents a heteroaryl group optionally substituted with one or more groups independently selected from R⁵;
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂;
t is 0, 1 or 2;
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷;
R³ represents H, C₁-C₃-alkyl or C₁-C₃-haloalkyl; and
R⁴ represents H, C₁-C₆-alkyl, C₃-C₆-cycloalkyl, C₁-C₄-haloalkyl or phenyl;
or,
R² and R³ together with the carbon to which they are attached form a C₃-C₆-cycloalkyl group or a 5- to 7-membered heterocycloalkyl group containing one heteroatom containing group selected from O, NR⁸, S, S(═O), S(═O)₂, S(═NR⁸)(═NR⁹) and S(═O)(═NR⁸); and
R⁴ represents H, C₁-C₆-alkyl, C₃-C₆-cycloalkyl, C₁-C₄-haloalkyl or phenyl;
or,
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷—NH₂, 4- to 7-membered heterocycloalkyl, —SR⁸, —S(═O)R⁸, —S(═O)₂R⁸ or —S(═O)(═NR⁸)R⁹;
R^{5a} represents R⁵, hydrogen or is absent;
R^{5b} represents hydrogen or a group selected from:
methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-, in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
wherein C₂-C₆-alkyl is optionally substituted with one or more substituents independently selected from the group consisting of:
halogen, hydroxy, C₁-C₃-alkoxy, C₁-C₃-haloalkoxy, oxo (═O), —NH₂, —N(H)R⁶, —N(R⁶)R⁷, —C(═O)OR⁸, —SR⁸, —S(═O)R⁸, —S(═O)₂R⁸ and —S(═O)(═NR⁸)R⁹;
wherein azetidin-3-yl and oxetan-3-yl are optionally substituted with one or two substituents independently selected from the group consisting of:
C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, and C₃-C₆-cycloalkyloxy;
wherein C₃-C₆-cycloalkyl and 5- to 7-membered heterocycloalkyl are optionally substituted with one or more substituents independently selected from the group consisting of:
hydroxy, halogen, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, C₃-C₆-cycloalkyloxy, —N(R⁵)R⁶, —C(═O)OH, oxo (═O), and —N(H)C(═O)—(C₁-C₃-alkyl);
wherein phenyl and 5- to 6-membered heteroaryl are optionally substituted with one or more substituents independently selected from the group consisting of:
halogen, C₁-C₃-alkyl, C₁-C₃-alkoxy-, C₁-C₃-haloalkoxy-, —N(H)R⁶, —N(R⁶)R⁷, —C(═O)OH and —C(═O)O(C₁-C₆-alkyl);
q is 0, 1, 2 or 3,
m is 0, 1, 2 or 3,
with the proviso that q+m is 2, 3 or 4; 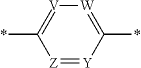
represents a group which is selected from: 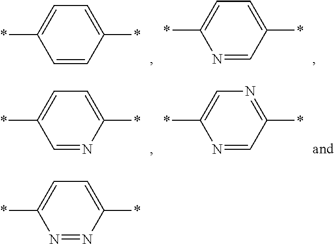
in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷;
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷;
R⁸, R⁹ represent, independently of each other, hydrogen, C₁-C₃-alkyl, C₃-C₆-cycloalkyl, phenyl or C₁-C₃-haloalkyl,
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷;
or an N-oxide, a salt, a tautomer or a stereoisomer of said compound, or a salt of said N-oxide, tautomer or stereoisomer.

2: The conjugate according to claim 1, wherein:
§¹ or §² represent the point of attachment to linker Z′, with the proviso that:
when linker Z′ is connected at §¹, then §² represents R^{5a}, and
when linker Z′ is connected at §², then linker Z′ is connected to a carbon or nitrogen atom of ring Het and §¹ represents R^{5b};
Het represents a heteroaryl group, optionally substituted with one or more groups independently selected from R⁵;
t is 0;
R² represents H, C₁-C₆-alkyl, C₃-C₆-cycloalkyl, C₁-C₄-haloalkyl or phenyl,
R³ represents H; and
R⁴ represents H, C₁-C₄-alkyl, or C₁-C₂-haloalkyl;
or,
R² represents H; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷, —NH₂, 4- to 7-membered heterocycloalkyl, —SR⁸, —S(═O)R⁸, —S(═O)₂R⁸ or —S(═O)(═NR⁸)R⁹;
R^{5a} represents R⁵, hydrogen or is absent;
R^{5b} represents hydrogen or a group selected from:
methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-,
in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
wherein C₂-C₆-alkyl is optionally substituted with one or more substituents independently selected from the group consisting of:
halogen, hydroxy, C₁-C₃-alkoxy, C₁-C₃-haloalkoxy, oxo (═O), —C(═O)OH and —N(R⁶)R⁷;
wherein C₃-C₆-cycloalkyl and 5- to 7-membered heterocycloalkyl are optionally substituted with one or more substituents independently selected from the group consisting of:
hydroxy, halogen, cyano, C₁-alkyl, C₁-haloalkyl, C₁-alkoxy, C₁-haloalkoxy, and oxo (═O);
wherein phenyl and 5- to 6-membered heteroaryl are optionally substituted with one or more substituents independently selected from the group consisting of:
halogen, C₁-C₃-alkyl, C₁-C₃-alkoxy-, C₁-C₃-haloalkoxy-, —C(═O)OH and —C(═O)O(C₁-C₆-alkyl);
q is 1,
m is 1, 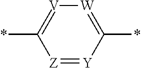
represents a group 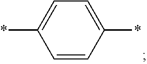 [SMILES: *c1ccc(*)cc1]
in which * and ^{#} represent the points of attachment of said group with the rest of the compound of formula (I),
R⁶, R⁷ represent, independently of each other, C₁-C₃-alkyl, C₃-C₆-cycloalkyl, phenyl or —C(═O)—(C₁-C₃-alkyl);
R⁸ represents hydrogen, C₁-C₃-alkyl, C₃-C₆-cycloalkyl, phenyl or C₁-C₃-haloalkyl;
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷;
or an N-oxide, a salt, a tautomer or a stereoisomer of said compound, or a salt of said N-oxide, tautomer or stereoisomer.

3: The conjugate according to claim 1, wherein:
§¹ or §² represent the point of attachment to linker Z′, with the proviso that:
when linker Z′ is connected at §¹, then §² represents R^{5a}, and
when linker Z′ is connected at §², then linker Z′ is connected to a nitrogen atom of ring Het and §¹ represents R^{5b};
Het represents a heteroaryl group optionally substituted with one or more groups independently selected from R⁵;
t is 0;
R² represents H,
R³ represents H; and
R⁴ represents H, C₁-alkyl, or C₁-haloalkyl;
or,
R² represents H; and
R³ and R⁴ together form a bond;
R⁵ represents, independently of each other, halogen, hydroxy, C₁-alkyl, 5- to 6-membered heterocycloalkyl, —SR⁸, —S(═O)R⁸ or —S(═O)₂R⁸;
R^{5a} represents hydrogen or is absent;
R^{5b} represents hydrogen or a group selected from:
methyl, C₂-C₃-alkyl,
wherein C₂-C₃-alkyl is optionally substituted with one or more substituents independently selected from the group consisting of:
halogen;
q is 1,
m is 1, 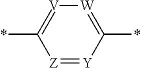
represents a group 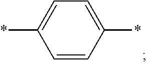 [SMILES: *c1ccc(*)cc1]
in which * and ^{#} represent the points of attachment of said group with the rest of the compound of formula (I),
R⁸ represents hydrogen, C₁-C₃-alkyl, or phenyl;
wherein said phenyl is optionally substituted with one or more substituents independently selected from the group consisting of:
C₁-alkyl;
or an N-oxide, a salt, a tautomer or a stereoisomer of said compound, or a salt of said N-oxide, tautomer or stereoisomer.

4: The conjugate according to a claim 1,
wherein:
§¹ or §² represent the point of attachment to linker Z′, with the proviso that:
when linker Z′ is connected at §¹, then §² represents R^{5a}, and
when linker Z′ is connected at §², then linker Z′ is connected to a nitrogen atom of ring Het and §¹ represents R^{5b};
Het represents a heteroaryl group optionally substituted with one or more groups independently selected from R⁵;
t is 0;
R² represents H,
R³ represents H; and
R⁴ represents H, or C₁-haloalkyl;
or,
R² represents H; and
R³ and R⁴ together form a bond;
R⁵ represents, independently of each other, C₁-alkyl, 6-membered heterocycloalkyl, or —S(═O)₂R⁸;
R^{5a} represents hydrogen or is absent;
R^{5b} represents hydrogen or a group selected from:
C₂-alkyl optionally substituted with one or more fluorine atoms;
q is 1,
m is 1, 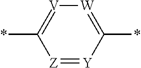
represents a group 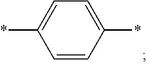 [SMILES: *c1ccc(*)cc1]
in which * and ^{#} represent the points of attachment of said group with the rest of the compound of formula (I),
R⁸ represents phenyl optionally substituted with one or more C₁-alkyl;
or an N-oxide, a salt, a tautomer or a stereoisomer of said compound, or a salt of said N-oxide, tautomer or stereoisomer.

5: The conjugate according to a claim 1, wherein the linker —Z′— represents one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²-L1-L2-§§
wherein
§¹, §² represent the attachment point to D;
§§ represents the attachment point to AB;
SG represents an in vivo cleavable group, L1 and L1′ represent, independently of each other, an in vivo non-cleavable organic group, and L2 represents an attachment group.

6: The conjugate according to claim 5, wherein the in vivo cleavable group SG represents a 2-8 oligopeptide group, or a disulfide, a hydrazone, a glycoside, an acetal or an aminal.

7: The conjugate according to claim 5, wherein L1 and L1′ represent, independently of each other, a straight-chain or branched hydrocarbon chain having 1 to 40 carbon atoms which may be interrupted once or more than once by one or more groups independently selected from:
—O—, —S—, —SO—, SO₂, —NH—, —CO—, —NMe-, —NHNH—, —SO₂NHNH—, —NHCO—, —CONH—, —CONHNH—, arylene groups, heteroarylene groups, straight C₁-C₆-alkylene groups, branched C₁-C₆-alkylene groups, C₃-C₇-cyclic alkylene groups and 5- to 10-membered heterocyclic groups having up to 4 heteroatoms selected from the group consisting of N, O, S, —SO— and —SO₂—;
optionally substituted with one or more substituents selected from the group consisting of halogen, —NHCONH₂, —COOH, —OH, —NH₂, NH—CNNH₂, sulphonamide, sulphone, sulphoxide and sulphonic acid.

8: The conjugate according to claim 5, wherein L2 represents: 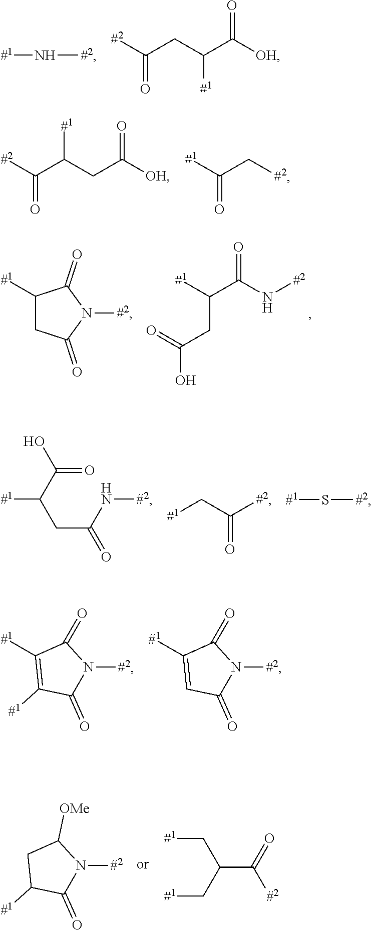
wherein
#¹ represents the attachment point to the binder,
#² represents the attachment point to the group L1, L1′ or SG.

9: The conjugate according to claim 1, wherein the linker —Z′— represents one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²-L1-L2-§§
wherein
§¹, §² represent the attachment point to D;
§§ represents the attachment point to AB;
SG represents a 2-8 oligopeptide group, or a disulfide, a hydrazone, a glycoside, an acetal or an aminal;
L1, L1′ represent, independently of each other, a straight-chain or branched hydrocarbon chain having 1 to 40 carbon atoms which may be interrupted once or more than once by one or more groups independently selected from:
—O—, —S—, —SO—, SO₂, —NH—, —CO—, —NMe-, —NHNH—, —SO₂NHNH—, —NHCO—, —CONH—, —CONHNH—, arylene groups, heteroarylene groups, straight C₁-C₆-alkylene groups, branched C₁-C₆-alkylene groups, C₃-C₇-cyclic alkylene groups and 5- to 10-membered heterocyclic groups having up to 4 heteroatoms selected from the group consisting of N, O and S, —SO— or —SO₂—;
optionally substituted with one or more substituents selected from the group consisting of halogen, —NHCONH₂, —COOH, —OH, —NH₂, NH—CNNH₂, sulphonamide, sulphone, sulphoxide or sulphonic acid;
L2 represents: 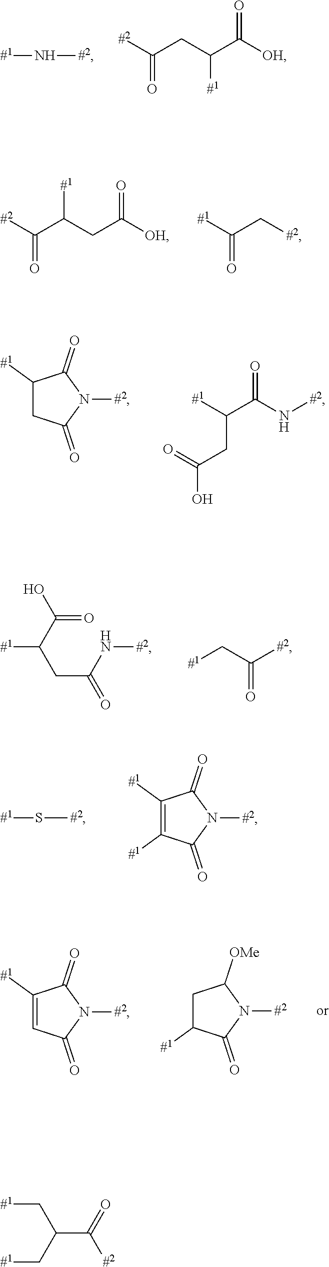
wherein
#¹ represents the attachment point to the binder,
#² represents the attachment point to the group L1, L1′ or SG.

10: The conjugate according to claim 6, wherein L2 represents one or more of the following three formulae: 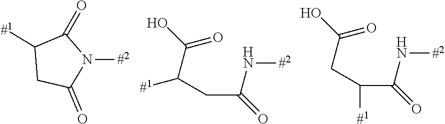
wherein
#¹ represents the attachment point to the binder,
#² represents the attachment point to the group L1, L1′ or SG,
wherein over 60% of the attachment points to the binder, in respect to the total number of attachments of the linker to the binder, are represented by one of the two structures: 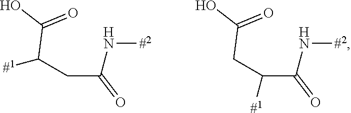
wherein, the amide group at #² is connected to L1, L1′ or SG via the group —CH₂—C(O)—.

11: The conjugate according to claim 6, wherein SG is a 2-8 oligopeptide.

12: The conjugate according to claim 11, wherein the 2-8 oligopeptide consists of amino acids selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, citrulline and valine.

13: The conjugate according to claim 5, wherein L1 and L1′ represent, independently of each other, a straight-chain or branched hydrocarbon chain having 1 to 20 carbon atoms which may be interrupted once or more than once by one or more groups independently selected from:
—O—, —NH—, —CO—, —NHCO—, —CONH—;
in which * and ^{#} represent the points of attachment of said group with the rest of the compound,
being optionally substituted with one or more substituents independently selected from the group consisting of —F, —Cl, —COOH, —OH, and —NH₂.

14: The conjugate according to claim 5, wherein L1 and L1′ represent, independently of each other, one of the general structures (iv) or (v):
(iv) -A′-(NR¹⁰CO)—B′—
(v) -A′-(CONR¹⁰)—B′—
wherein:
A′ represents C₁-C₆ alkyl, (C₁-C₂ alkyl)-(phenylene), and (C₁-C₃ alkyl)-(NR¹¹)—(C₂ alkyl);
optionally substituted with one or more substituents independently selected from —F and —Cl;
B′ represents a straight-chain or branched hydrocarbon chain having 1 to 20 carbon atoms which may be interrupted once or more than once by one or more groups independently selected from: —O—, —NH—, —CO—, —NHCO—, and —CONH—;
optionally substituted with —COOH;
R¹⁰, R¹¹ represent, independently of each other hydrogen or C₁-C₃ alkyl; or
R¹⁰, R¹¹ together with the nitrogens to which they are attached form a 6-membered nitrogen containing heterocycloalkyl group.

15: The conjugate according to claim 5, wherein the linker —Z′— represents, one of the general structures (vi) to (vii):
(vi) §¹-A²-(NR¹⁰—SG′-CO)—B²-L2-§§
(vii) §²-A²-(NR¹⁰-SG′-CO)—B²-L2-§§
§¹, §² represent the attachment point to D;
§§ represents the attachment point to AB;
L2 is 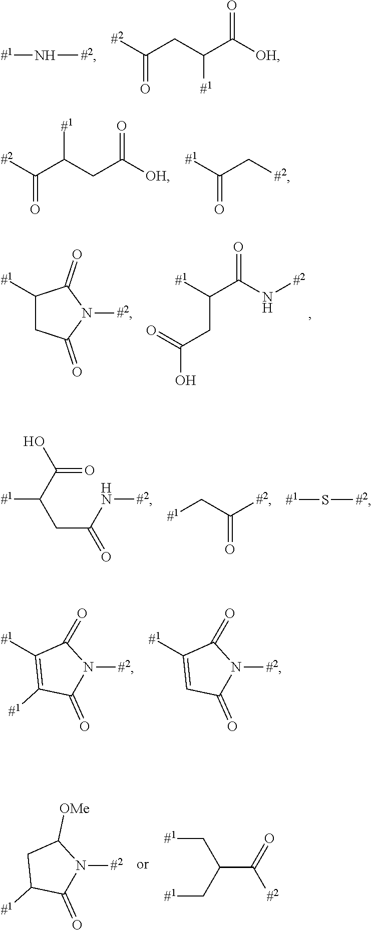
wherein
#¹ represents the attachment point to the binder,
#² represents the attachment point to the group L1, L1′ or SG:
SG′ is optionally present and, when present, represents:
SG as defined in claim 5, or
one amino acid (lysine, asparagine) optionally substituted with —[CH₂—CH₂O]ₒCH₃; —C(═O)[CH₂—CH₂O]ₒCH₃; —NHC(═O)[CH₂—CH₂O]ₒCH₃,
o represents an integer from 3 to 9;
A² represents C₂-C₆-alkyl; optionally substituted with one or more substituents independently selected from —F, —Cl and —COOH;
B² represents a straight-chain or branched hydrocarbon chain having 1 to 20 carbon atoms which may be interrupted once or more than once by one or more groups independently selected from —O—, —NH—, —CO—, —NHCO—, and —CONH—;
optionally substituted with —COOH; and
R¹⁰ represents hydrogen or C₁-C₃ alkyl.

16: A conjugate of general formula (II): 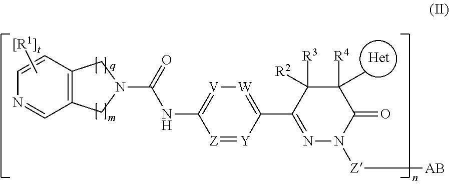
wherein AB stands for a binder, Z′ stands for a linker, n stands for a number between 1 and 50,
wherein:
wherein R¹, R², R³, R⁴, Het, t, q, m, V, W, Z and Y are as defined in claim 1:
—Z′— represents one of the following general structures (i) to (iii):
(i) §¹-L1-SG-L2-§§
(ii) §¹-L1-SG-L1′-L2-§§
(iii) §¹-L1-L2-§§
wherein
§¹ represents the attachment point to the pyridazinone ring;
§§ represents the attachment point to AB;
SG represents a 2-8 oligopeptide group, or a disulfide, a hydrazone, a glycoside, an acetal or an aminal;
L1, L1′ represent, independently of each other, a straight-chain or branched hydrocarbon chain having 1 to 40 carbon atoms which may be interrupted once or more than once by one or more of —O—, —S—, —SO—, SO₂, —NH—, —CO—, —NMe-, —NHNH—, —SO₂NHNH—, —NHCO—, —CONH—, —CONHNH—, arylene groups, heteroarylene groups, cyclic alkylene groups and 5- to 10-membered heterocyclic groups having up to 4 heteroatoms selected from the group consisting of N, O and S, —SO— or —S02-;
in which * and ^{#} represent the points of attachment of said group with the rest of the compound,
optionally substituted with one or more substituents selected from the group consisting of halogen, —NHCONH₂, —COOH, —OH, —NH₂, NH—CNNH₂, sulphonamide, sulphone, sulphoxide and sulphonic acid;
L2 represents: 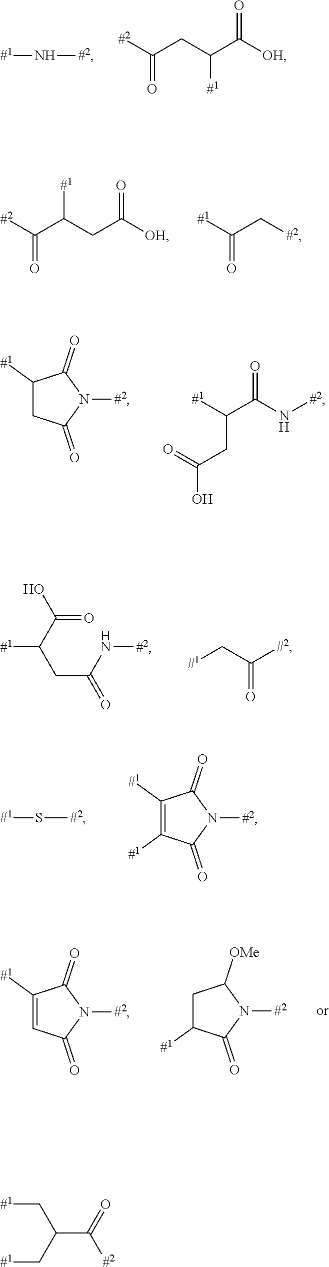
wherein
#¹ represents the attachment point to the binder,
#² represents the attachment point to the group L1, L1′ or SG;
or an enantiomer, a diastereomer, a salt, a solvate, or salt of the solvate thereof.

17: The conjugate according to claim 1, which is selected from the group consisting of: 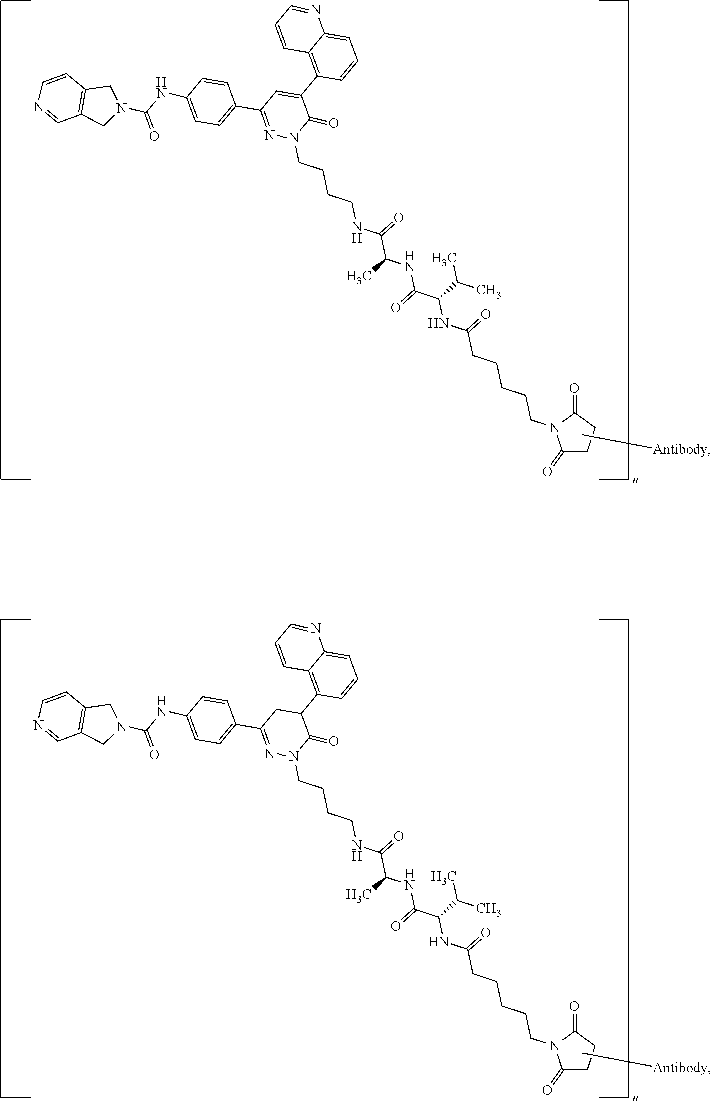 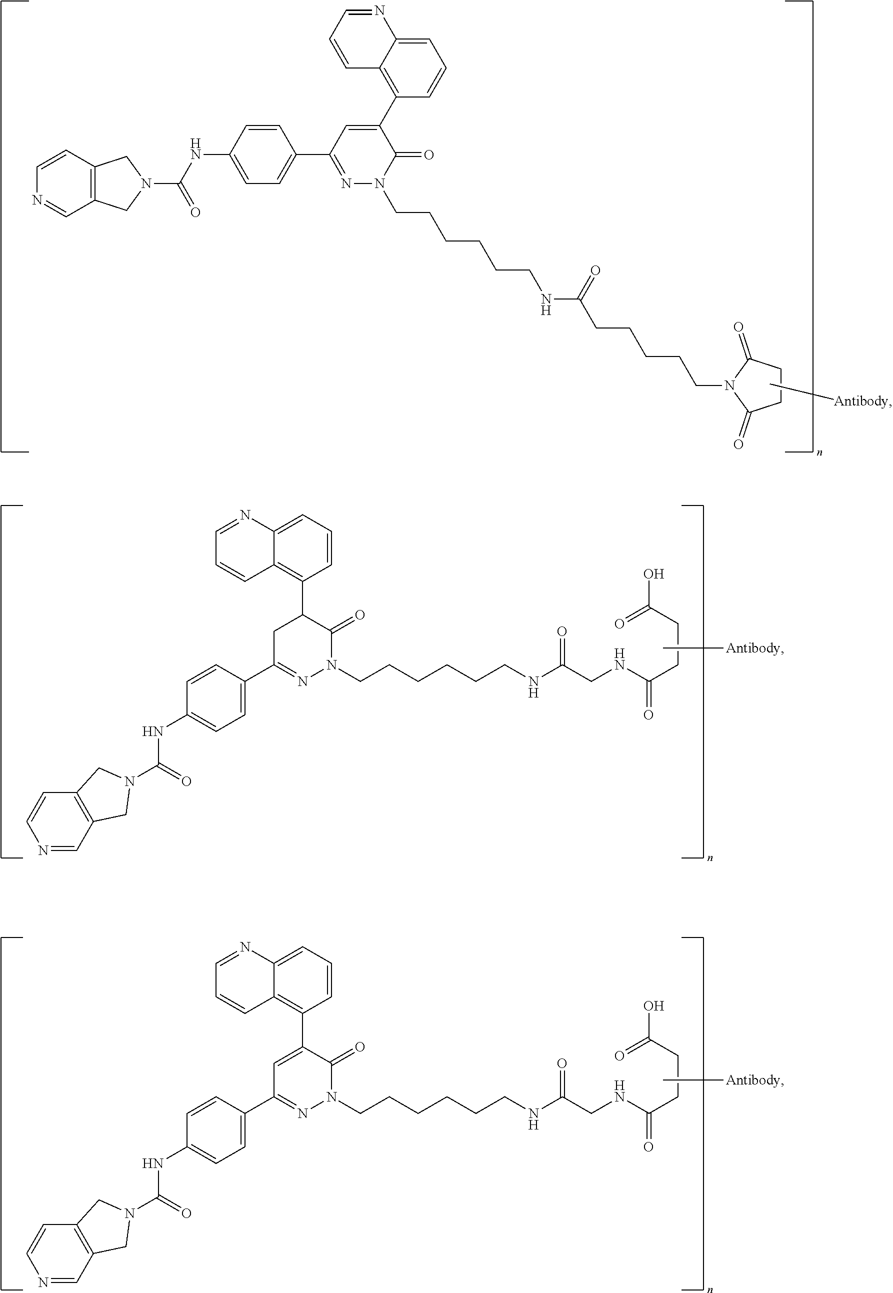 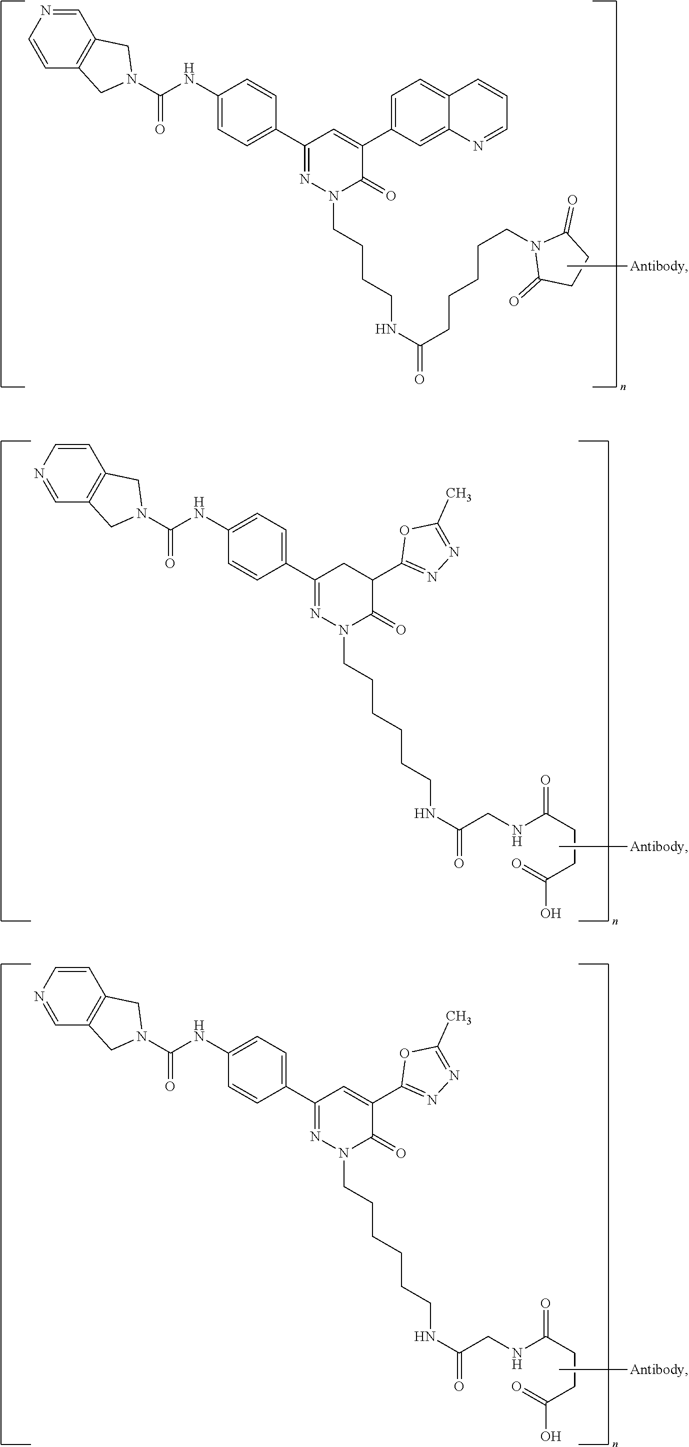 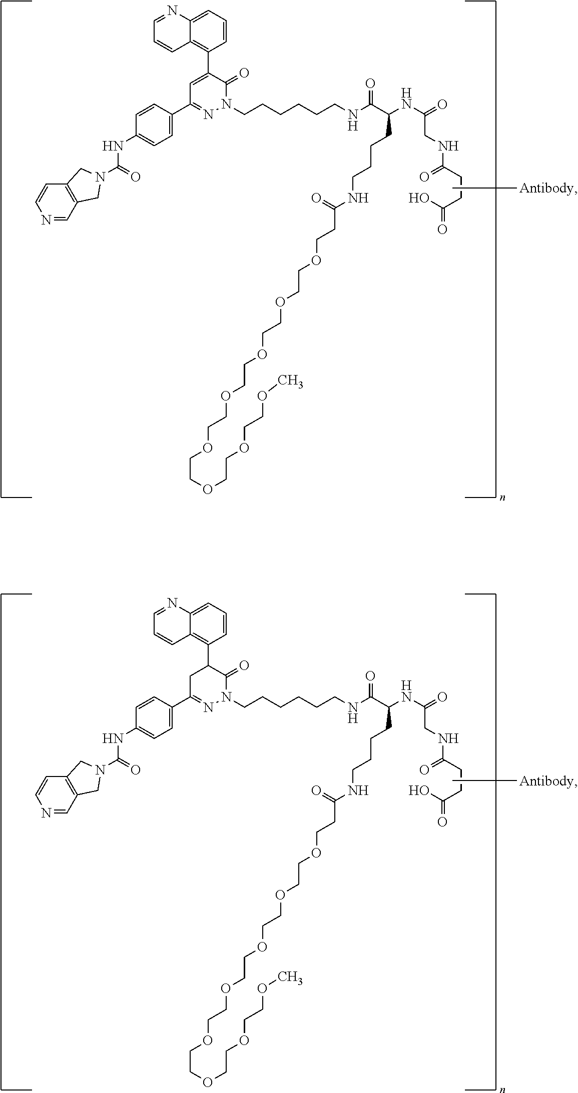 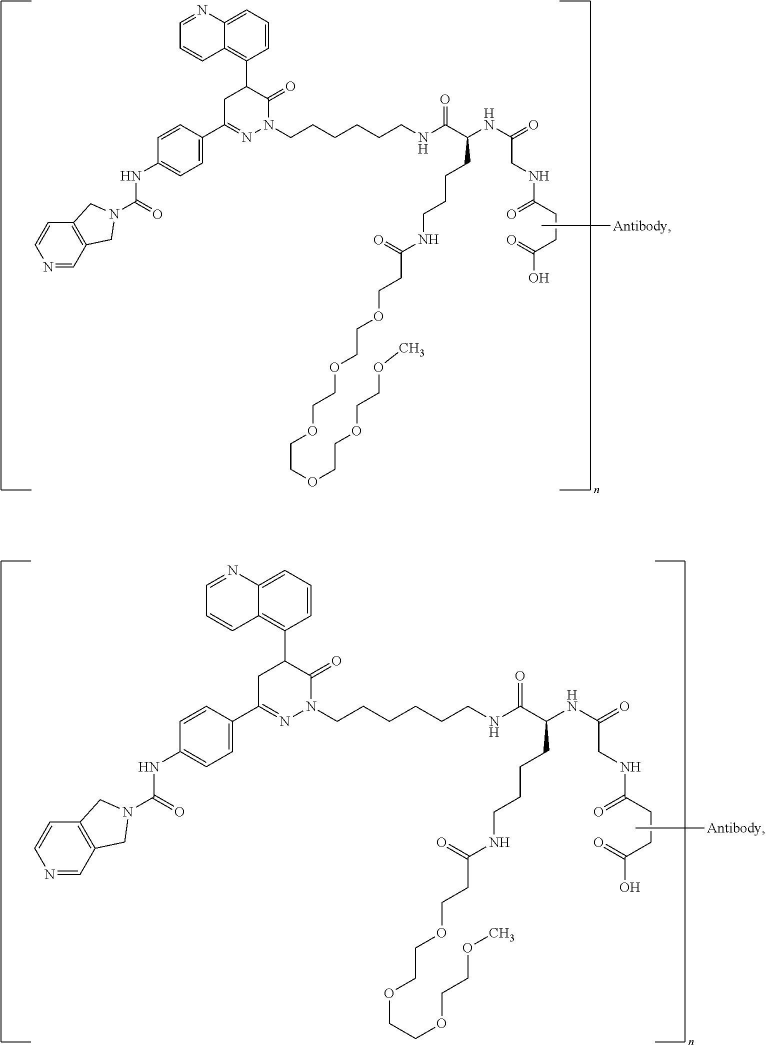 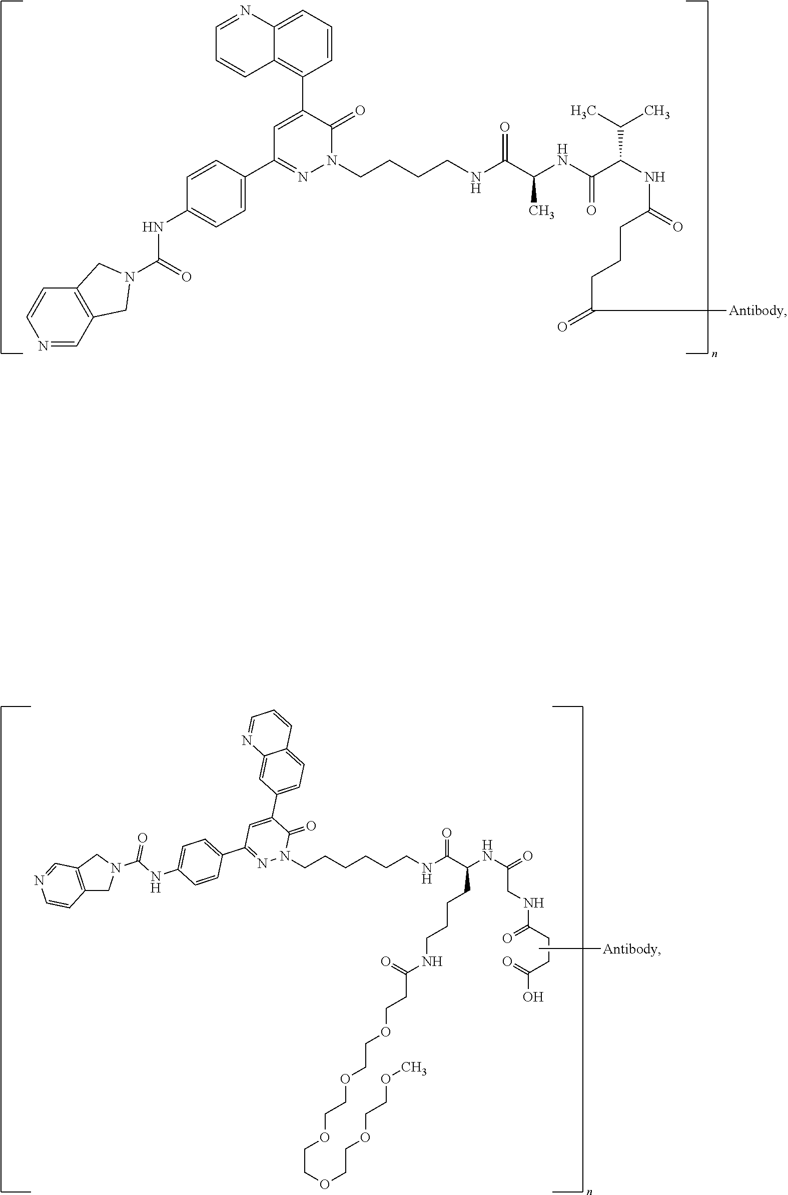 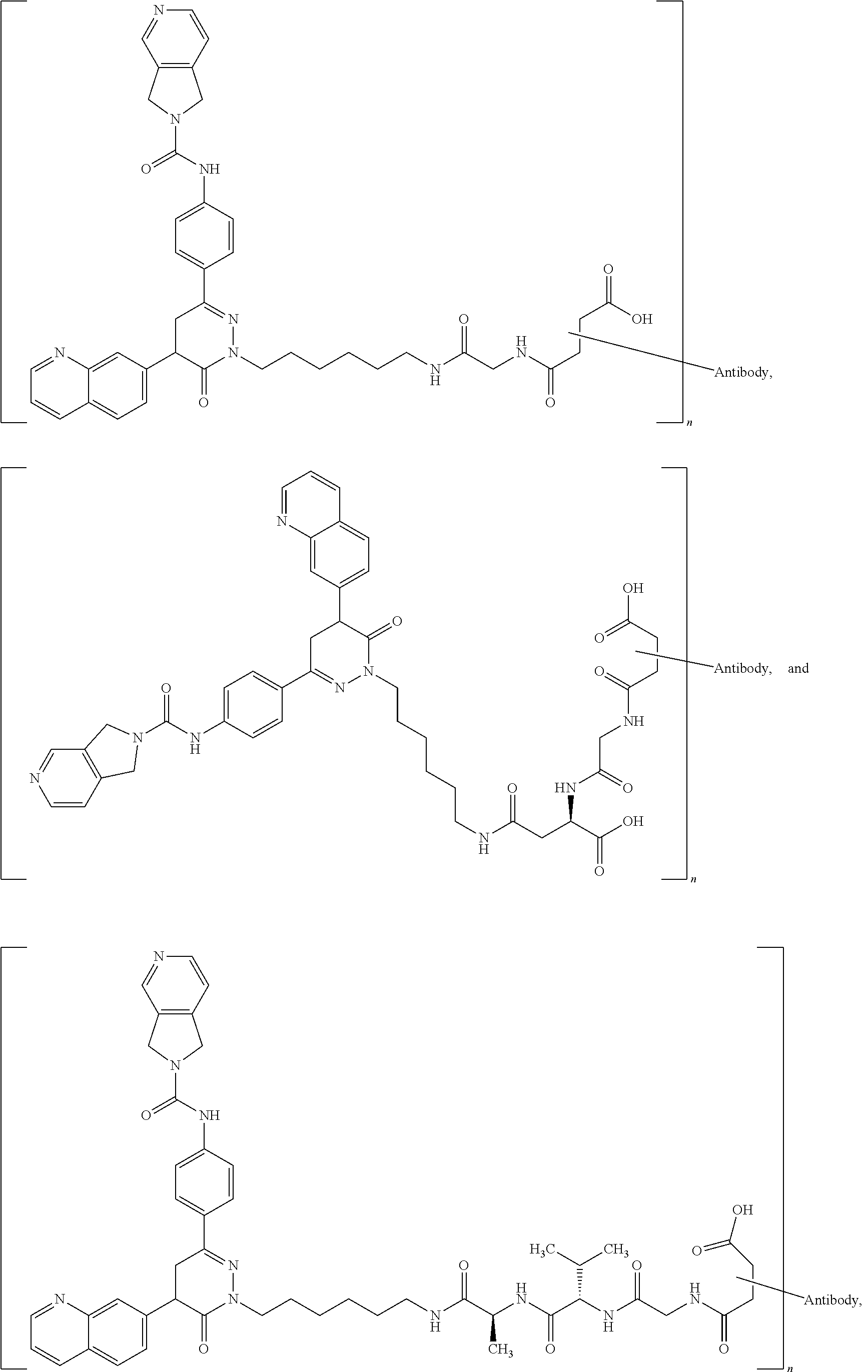
wherein n is a number from 1 to 50, and the antibody is selected from an anti-HER2-antibody, an anti-CXCR5-antibody, an anti-B7H3-antibody, an anti-C4.4a-antibody, or an antigen binding fragment thereof.

18: A conjugate of general formula (III): 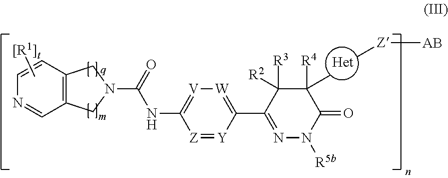
wherein AB stands for a binder, Z′ stands for a linker, n stands for a number between 1 and 50,
wherein:
wherein R¹, R², R³, R⁴, R^{5b}, Het, t, q, m, V, W, Z and Y are as defined in claim 1;
—Z′— represents one of the following general structures (i) to (iii):
(i) §²-L1-SG-L2-§§
(ii) §²-L1-SG-L1′-L2-§§
(iii) §²-L1-L2-§§
wherein
§² represents the attachment point to ring Het;
§§ represents the attachment point to AB;
SG represents a 2-8 oligopeptide group, or a disulfide, a hydrazone, a glycoside, an acetal or an aminal;
L1, L1′ represent, independently of each other, a straight-chain or branched hydrocarbon chain having 1 to 40 carbon atoms which may be interrupted once or more than once by one or more of —O—, —S—, —SO—, SO₂, —NH—, —CO—, —NMe-, —NHNH—, —SO₂NHNH—, —NHCO—, —CONH—, —CONHNH—, arylene groups, heteroarylene groups, cyclic alkylene groups and 5- to 10-membered heterocyclic groups having up to 4 heteroatoms selected from the group consisting of N, O and S, —SO— or —SO₂—;
in which * and ^{#} represent the points of attachment of said group with the rest of the compound,
optionally substituted with one or more substituents selected from the group consisting of halogen, —NHCONH₂, —COOH, —OH, —NH₂, NH—CNNH₂, sulphonamide, sulphone, sulphoxide and sulphonic acid;
L2 represents: 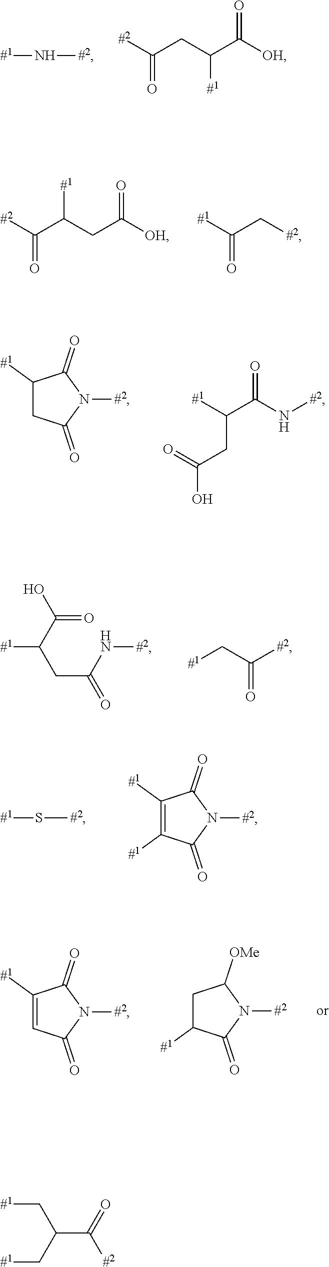
wherein
#¹ represents the attachment point to the binder,
#² represents the attachment point to the group L1, L1′ or SG;
or an enantiomer, a diastereomer, a salt, a solvate, or salt of the solvate thereof.

19: The conjugate according to claim 1, which is selected from the group consisting of: 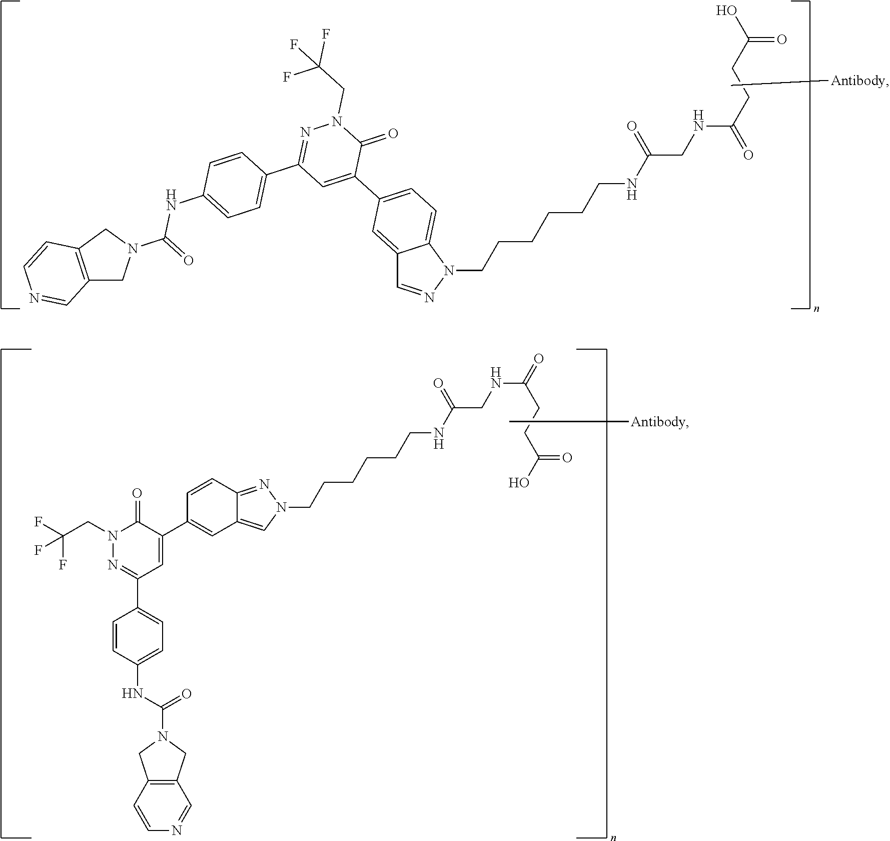 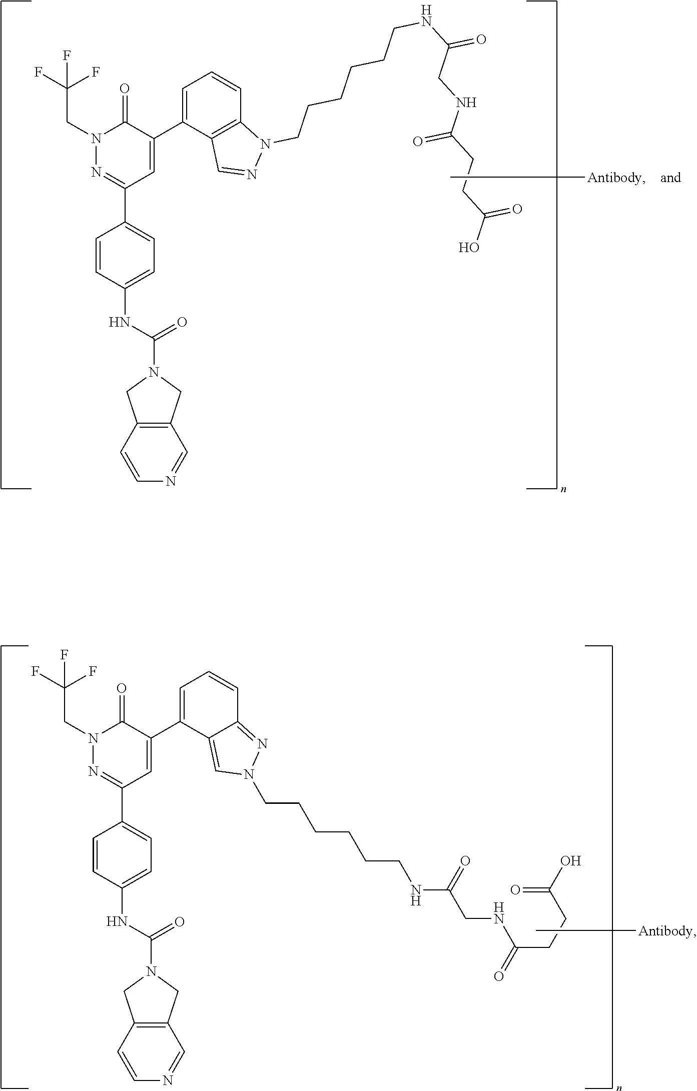
wherein n is a number from 1 to 50, and the antibody is selected from an anti-HER2-antibody, an anti-CXCR5-antibody, an anti-B7H3-antibody, an anti-C4.4a-antibody, or an antigen binding fragment thereof.

20. (canceled)

21: The conjugate according to claim 1, wherein binder AB is an antibody is selected from an anti-HER2-antibody, an anti-CXCR5-antibody, an anti-B7H3-antibody, and an anti-C4.4a-antibody, or an antigen binding fragment thereof.

22: The conjugate according to claim 1, wherein linker Z′ represents one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²-L1-L2-§§
wherein
§¹, §² represent the attachment point to D;
§§ represents the attachment point to AB;
L1 and L1′, independently of one another, are as defined in any one of the rows of Table A or Table B;
r independently of one another represents a number from 1 to 20, 0; and
SG is an in vivo cleavable group, and
L2 represents an attachment group.

23: The conjugate according to claim 1, wherein linker Z′ represents one of the following general structures (i) to (iii):
(i) §¹-L1-SG-L2-§§ or §²-L1-SG-L2-§§
(ii) §¹-L1-SG-L1′-L2-§§ or §²-L1-SG-L1′-L2-§§
(iii) §¹-L1-L2-§§ or §²-L1-L2-§§
wherein
§¹, §² represent the attachment point to D;
§§ represents the attachment point to AB;
and L1, SG, L1′ and L2 are as defined in any one of the rows of Table C or Table D.

24: The conjugate according to claim 1, wherein SG comprises (C-terminus)-Ala-Val-(N-terminus) or (C-terminus)-Cit-Val-(N-terminus).

25: The conjugate according to claim 1, wherein Het represents a heteroaryl group selected from quinolin-5-yl, 1,3,4-oxadiazol-2-yl, 1H-indazol-5-yl, 1H-indazol-4-yl, quinolin-7-yl, or 1H-benzimidazol-4-yl, said group being optionally substituted with one or more groups independently selected from R⁵;

26: The conjugate according to claim 1, wherein the linker Z′ is bound to a cysteine side chain on the binder AB.

27: The conjugate according to claim 1, wherein the binder or a derivative thereof is a binding peptide or -protein or a derivative of a binding peptide or -protein.

28: The conjugate according to claim 1, wherein each molecule of the active component is binding to different amino acids of the binding peptide or -protein or their derivatives respectively, via a linker.

29: The conjugate according to claim 1, wherein the conjugate averages 1.2 to 50 molecules of the active components per binder.

30: The conjugate according to claim 28, wherein the binding peptide or protein represents an antibody or wherein the derivative of the binding peptide or -protein comprises one of the following groups:

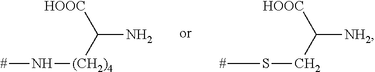

31: The conjugate according to claim 1, wherein the binder binds to a cancer target-molecule.

32: The conjugate according to claim 31, wherein the binder is binding to an extracellular target molecule.

33: The conjugate according to claim 32, wherein the binder, after binding to the extracellular target molecule, is internalized in the expressing cell of the target molecule and is processed intracellularly.

34: The conjugate according to claim 1, wherein the binding peptide or -protein is a human, humanized or chimeric monoclonal antibody, or an antigen-binding fragment thereof.

35: The conjugate according to claim 34, wherein the binding peptide or -protein is, an anti-HER2-antibody, an anti-CXCR5-antibody, an anti-B7H3-antibody, an anti-C4.4a-antibody; or an antigen binding fragment thereof.

36: A metabolite obtainable by the cleavage of any of the conjugates as defined in claim 1.

37: The metabolite according to claim 36, wherein the metabolite does not comprise a cysteine and/or a lysine residue of the binder protein or peptide.

38: The metabolite according to claim 36, which is selected from the group consisting of:
S-{1-[6-({6-[3-{4-[(1,3-Dihydro-2H-pyrrolo[3,4-c]pyridine-2-carbonyl)amino]phenyl}-6-oxo-5-(quinolin-5-yl)pyridazin-1(6H)-yl]hexyl}amino)-6-oxohexyl]-2,5-dioxopyrrolidin-3-yl}-L-cysteine,
2-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic acid,
3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic acid,
2-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]amino}-4-oxobutanoic acid,
3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]hexyl}amino)-2-oxoethyl]amino}-4-oxobutanoic acid,
2-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}amino)-2-oxoethyl]amino}-4-oxobutanoic acid,
3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic acid,
N-[2-{[(2R)-2-amino-2-carboxyethyl]sulfanyl}-3-carboxypropanoyl]glycyl-N-{6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}-N⁶-(26-oxo-2,5,8,11,14,17,20,23-octaoxahexacosan-26-yl)-L-lysinamide, and
N-[3-{[(2R)-2-amino-2-carboxyethyl]sulfanyl}-3-carboxypropanoyl]glycyl-N-{6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}-N⁶-(26-oxo-2,5,8,11,14,17,20,23-octaoxahexacosan-26-yl)-L-lysinamide,
or an N-oxide, a salt, a tautomer or a stereoisomer of said metabolite, or a salt of said N-oxide, tautomer or stereoisomer.

39: A compound selected from: 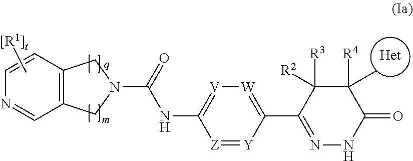
wherein
Het represents a heteroaryl group optionally substituted with one or more groups independently selected from R⁵;
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂;
t is 0, 1 or 2;
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷;
R³ represents H, C₁-C₃-alkyl or C₁-C₃-haloalkyl; and
R⁴ represents H, C₁-C₆-alkyl, C₃-C₆-cycloalkyl, C₁-C₄-haloalkyl or phenyl;
or,
R² and R³ together with the carbon to which they are attached form a C₃-C₆-cycloalkyl group or a 5- to 7-membered heterocycloalkyl group containing one heteroatom containing group selected from O, NR⁸, S, S(═O), S(═O)₂, S(═NR⁸)(═NR⁹) and S(═O)(═NR⁸); and
R⁴ represents H, C₁-C₆-alkyl, C₃-C₆-cycloalkyl, C₁-C₄-haloalkyl or phenyl;
or,
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷; and
R³ and R⁴ together form a bond;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷—NH₂, 4- to 7-membered heterocycloalkyl, —SR⁸, —S(═O)R⁸, —S(═O)₂R⁸ or —S(═O)(═NR⁸)R⁹;
q is 0, 1, 2 or 3,
m is 0, 1, 2 or 3,
with the proviso that q+m is 2, 3 or 4; 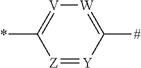
represents a group which is selected from: 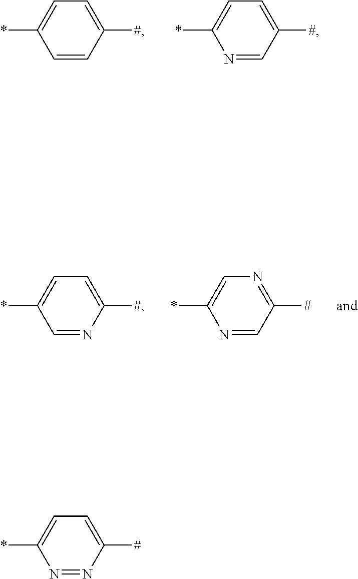
in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷;
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷;
R⁸, R⁹ represent, independently of each other, hydrogen, C₁-C₃-alkyl, C₃-C₆-cycloalkyl, phenyl or C₁-C₃-haloalkyl,
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷; 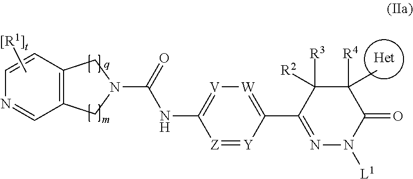
wherein R¹, R², R³, R⁴, Het, t, q, m, V, W, Z and Y are as defined above and L1 is an in vivo non-cleavable organic group, wherein
when Het represents a NH containing heteroaryl groups, said NH is optionally protected with an amino protecting group; 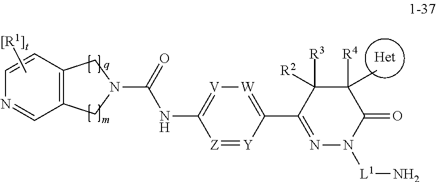
wherein R¹, R², R³, R⁴, Het, L¹, t, q, m, V, W, Z and Y are as defined above; 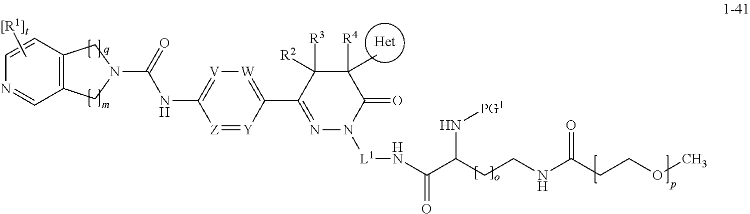
wherein R¹, R², R³, R⁴, Het, L¹, t, q, m, V, W, Z and Y are as defined above and o is 1 to 5 and p is 1 to 12, PG¹ represents an amine protecting group; 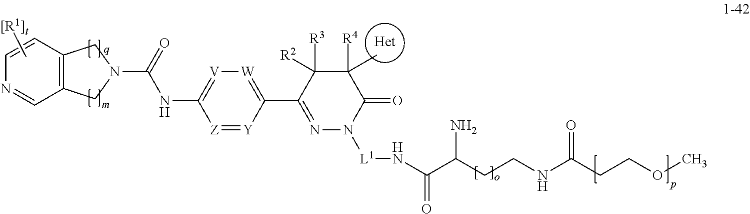
R¹, R², R³, R⁴, Het, L¹, t, q, m, V, W, Z and Y are as defined above and o is 1 to 5 and p is 1 to 12; 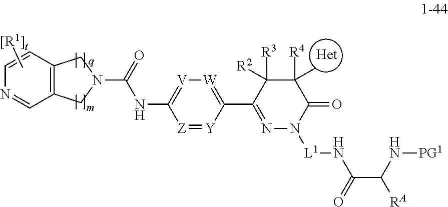
wherein R¹, R², R³, R⁴, Het, L¹, t, q, m, V, W, Z and Y are as defined above, PG1 represents an amine protecting group, and
R^{A} represents hydrogen (glycine) or a group selected from:
—CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (ornithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl- (histidine), (1H-indol-3-yl)methyl- (tryptophan), —CH₂NH₂ (2,3-diaminopropanoic acid), and —(CH₂)₂NH₂ (2,4-diaminobutanoic acid); 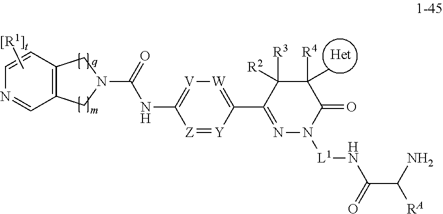
wherein R¹, R², R³, R⁴, Het, L¹, t, q, m, V, W, Z and Y are as defined above, and
R^{A} represents hydrogen (glycine) or a group selected from:
—CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (ornithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl- (histidine), (1H-indol-3-yl)methyl- (tryptophan), —CH₂NH₂ (2,3-diaminopropanoic acid), and —(CH₂)₂NH₂ (2,4-diaminobutanoic acid); 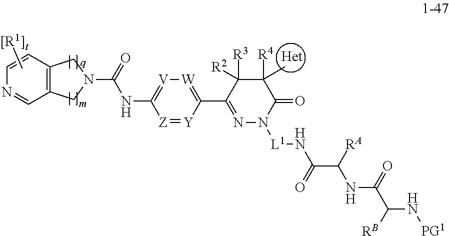
wherein R¹, R², R³, R⁴, Het, L¹, t, q, m, V, W, Z and Y are as defined above, PG1 represents an amine protecting group, and
R^{A} and R^{B}, independently of each other, represent hydrogen (glycine) or a group selected from:
—CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (ornithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl- (histidine), (1H-indol-3-yl)methyl- (tryptophan), —CH₂NH₂ (2,3-diaminopropanoic acid), and —(CH₂)₂NH₂ (2,4-diaminobutanoic acid); 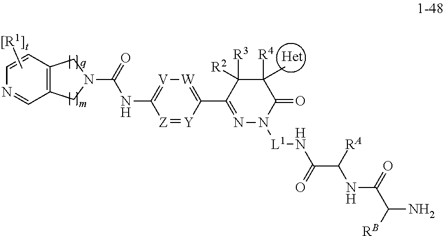
wherein R¹, R², R³, R⁴, Het, L¹, t, q, m, V, W, Z and Y are as defined above, and
R^{A} and R^{B}, independently of each other, represent hydrogen (glycine) or a group selected from:
—CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (ornithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl- (histidine), (1H-indol-3-yl)methyl- (tryptophan), —CH₂NH₂ (2,3-diaminopropanoic acid), and —(CH₂)₂NH₂ (2,4-diaminobutanoic acid); 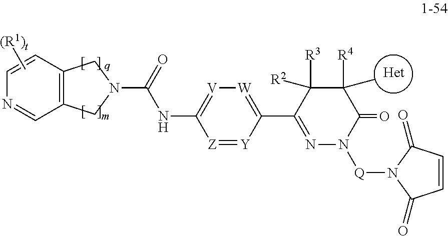
wherein R¹, R², R³, R⁴, Het, t, q, m, V, W, Z and Y are as defined above and Q represents one of the following general structures (i) to (iii):
(i) §-L1-SG-§§
(ii) §-L1-SG-L1′-§§
(iii) §-L1-§§
wherein
§ represents the attachment point to the pyridazinone ring;
§§ represents the attachment point to the maleimide group;
SG represents an in vivo cleavable group, L1 and L1′ represent, independently of each other, an in vivo non-cleavable organic group, as defined in any one of the preceding claims; 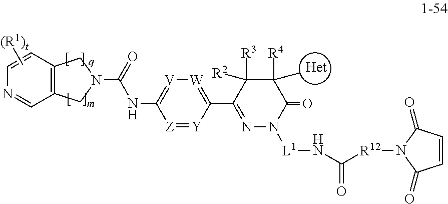
wherein R¹, R², R³, R⁴, Het, L¹, t, q, m, V, W, Z and Y are as defined in any one of the preceding claims and R¹² is C₁-C₁₀-alkyl; 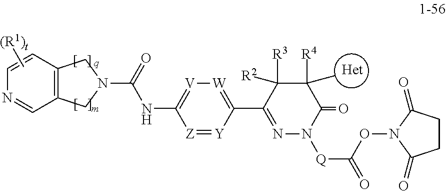
wherein R¹, R², R³, R⁴, Het, t, q, m, V, W, Z and Y are as defined above and Q represents one of the following general structures (i) to (iii):
(i) §-L1-SG-§§
(ii) §-L1-SG-L1′-§§
(iii) §-L1-§§
wherein
§ represents the attachment point to the pyridazinone ring;
§§ represents the attachment point to the carbonyl group;
SG represents an in vivo cleavable group, L1 and L1′ represent, independently of each other, an in vivo non-cleavable organic group; and 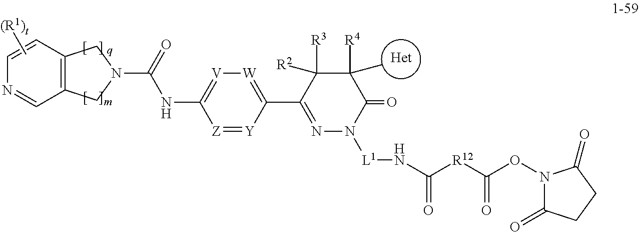
wherein R¹, R², R³, R⁴, Het, L¹, t, q, m, V, W, Z and Y are as defined above and wherein R¹² is C₁-C₁₀-alkyl,
or an N-oxide, a salt, a tautomer or a stereoisomer of said compound, or a salt of said N-oxide, tautomer or stereoisomer.

40: The compound according to claim 39, which is selected from the group consisting of:
N-{4-[6-Oxo-5-(quinolin-5-yl)-1,4,5,6-tetrahydropyridazin-3-yl]phenyl}-1,3-dihydro-2H-pyrrolo[3,4-c]pyridine-2-carboxamide,
tert-Butyl {4-[-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}carbamate,
N-{4-[1-(4-Aminobutyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}carbamate,
N-{4-[1-(6-Aminohexyl)-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[6-Oxo-5-(quinolin-5-yl)-1,6-dihydropyridazin-3-yl]phenyl}-1,3-dihydro-2H-pyrrolo[3,4-c]pyridine-2-carboxamide,
N-{4-[1-(4-Aminobutyl)-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[1-(6-Aminohexyl)-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[5-(5-Methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide,
tert-Butyl {4-[3-{4-[(1,3-dihydro-2H-pyrrolo[3,4-c]pyridin-2-ylcarbonyl)amino]phenyl}-5-(5-methyl-1,3,4-oxadiazol-2-yl)-6-oxopyridazin-1(6H)-yl]butyl}carbamate,
N-{4-[1-(4-Aminobutyl)-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
N-{4-[5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-5-(5-methyl-1,3,4-oxadiazol-2-yl)-6-oxopyridazin-1(6H)-yl]hexyl}carbamate,
N-{4-[1-(6-Aminohexyl)-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
N-{4-[1-(6-Aminohexyl)-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide,
N-{4-[(5R)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
tert-Butyl {4-[(5R)-3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]butyl}carbamate,
N-{4-[(5R)-1-(4-aminobutyl)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
N-{4-[6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
tert-Butyl {6-[(3-{4-[(1,3-dihydro-2H-pyrrolo[3,4-c]pyridine-2-carbonyl)amino]phenyl}-6-oxo-5-(quinolin-5-yl)-5-(trifluoromethyl)-5,6-dihydropyridazin-1(4H)-yl]hexyl}carbamate,
N-{4-[1-(6-Aminohexyl)-6-oxo-5-(quinolin-5-yl)-5-(trifluoromethyl)-1,4,5,6-tetrahydropyridazin-3-yl]phenyl}-1,3-dihydro-2H-pyrrolo[3,4-c]pyridine-2-carboxamide,
N-[4-(5-{1-[Oxan-2-yl]-1H-indazol-5-yl}-6-oxo-1,6-dihydropyridazin-3-yl)phenyl]-1,3-dihydro-2H-pyrrolo[3,4-c]pyridine-2-carboxamide,
N-{4-[5-(1H-indazol-5-yl)-6-oxo-1,6-dihydropyridazin-3-yl]phenyl}-1,3-dihydro-2H-pyrrolo[3,4-c]pyridine-2-carboxamide,
N-{4-[5-(1H-indazol-5-yl)-6-oxo-1,6-dihydropyridazin-3-yl]phenyl}-1,3-dihydro-2H-pyrrolo[3,4-c]pyridine-2-carboxamide-hydrogen chloride,
N-{4-[5-(1H-indazol-5-yl)-6-oxo-1,4,5,6-tetrahydropyridazin-3-yl]phenyl}-1,3-dihydro-2H-pyrrolo[3,4-c]pyridine-2-carboxamide,
N-[4-(5-{1-[Oxan-2-yl]-1H-indazol-4-yl}-6-oxo-1,6-dihydropyridazin-3-yl)phenyl]-1,3-dihydro-2H-pyrrolo[3,4-c]pyridine-2-carboxamide,
N-{4-[5-(1H-indazol-4-yl)-6-oxo-1,6-dihydropyridazin-3-yl]phenyl}-1,3-dihydro-2H-pyrrolo[3,4-c]pyridine-2-carboxamide,
N-{4-[5-{1-[oxan-2-yl]-1H-indazol-4-yl}-6-oxo-1,4,5,6-tetrahydropyridazin-3-yl]phenyl}-1,3-dihydro-2H-pyrrolo[3,4-c]pyridine-2-carboxamide,
N-{4-[5-(1H-indazol-4-yl)-6-oxo-1,4,5,6-tetrahydropyridazin-3-yl]phenyl}-1,3-dihydro-2H-pyrrolo[3,4-c]pyridine-2-carboxamide,
N-(4-{5-[1-(4-Methylbenzene-1-sulfonyl)-1H-indol-5-yl]-6-oxo-1,6-dihydropyridazin-3-yl}phenyl)-1,3-dihydro-2H-pyrrolo[3,4-c]pyridine-2-carboxamide,
N-{4-[5-(1H-indol-5-yl)-6-oxo-1,6-dihydropyridazin-3-yl]phenyl}-1,3-dihydro-2H-pyrrolo[3,4-c]pyridine-2-carboxamide,
N-{4-[5-(1H-Indol-5-yl)-6-oxo-1,4,5,6-tetrahydropyridazin-3-yl]phenyl}-1,3-dihydro-2H-pyrrolo[3,4-c]pyridine-2-carboxamide,
N-{4-[5-{1-[Oxan-2-yl]-1H-indazol-5-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-{1-[Oxan-2-yl]-1H-indazol-4-yl}-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[6-Oxo-5-(quinolin-7-yl)-1,6-dihydropyridazin-3-yl]phenyl}-1,3-dihydro-2H-pyrrolo[3,4-c]pyridine-2-carboxamide,
tert-Butyl {4-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]butyl}carbamate,
N-{4-[1-(4-Aminobutyl)-6-oxo-5-(quinolin-7-yl)-1,6-dihydropyridazin-3-yl]phenyl}-1,3-dihydro-2H-pyrrolo[3,4-c]pyridine-2-carboxamide,
N-(4-{5-[1-(4-Methylbenzene-1-sulfonyl)-1H-benzimidazol-4-yl]-6-oxo-1,6-dihydropyridazin-3-yl}phenyl)-1,3-dihydro-2H-pyrrolo[3,4-c]pyridine-2-carboxamide,
N-{4-[5-(1H-Benzimidazol-4-yl)-6-oxo-1,6-dihydropyridazin-3-yl]phenyl}-1,3-dihydro-2H-pyrrolo[3,4-c]pyridine-2-carboxamide,
tert-Butyl [(2S)-1-({4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}amino)-1-oxopropan-2-yl]carbamate,
[(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]ammonium trifluoroacetate,
N-(tert-Butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide,
[(1S)-1-[[(1S)-2-[4-[3-[4-(1,3-Dihydropyrrolo[3,4-c]pyridine-2-carbonylamino)phenyl]-6-oxo-5-(5-quinolyl)pyridazin-1-yl]butylamino]-1-methyl-2-oxo-ethyl]carbamoyl]-2-methyl-propyl]ammonium trifluoroacetate,
tert-Butyl [(2S)-1-({4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}amino)-1-oxopropan-2-yl]carbamate,
[(1S)-2-[4-[3-[4-(1,3-Dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]ammonium trifluoroacetate,
N-(tert-Butoxycarbonyl)-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide,
[(1S)-1-[[(1S)-2-[4-[3-[4-(1,3-Dihydropyrrolo[3,4-c]pyridine-2-carbonylamino)phenyl]-6-oxo-5-(5-quinolyl)-4,5-dihydropyridazin-1-yl]butylamino]-1-methyl-2-oxo-ethyl]carbamoyl]-2-methyl-propyl]ammonium trifluoroacetate,
2 or 3-{[(2R)-2-Amino-2-carboxyethyl]sulfanyl}-4-{[2-({6-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]hexyl}amino)-2-oxoethyl]amino}-4-oxobutanoic acid,
(9H-fluoren-9-yl)methyl {(32S)-40-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]-26,33-dioxo-2,5,8,11,14,17,20,23-octaoxa-27,34-diazatetracontan-32-yl}carbamate,
N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,6-dihydropyridazin-3-yl]phenyl}-1,3-dihydro-2H-pyrrolo[3,4-c]pyridine-2-carboxamide,
(9H-fluoren-9-yl)methyl {(32S)-40-[(5S)-3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-26,33-dioxo-2,5,8,11,14,17,20,23-octaoxa-27,34-diazatetracontan-32-yl}carbamate,
N-{4-[6-oxo-1-(6-{[N⁶-(26-oxo-2,5,8,11,14,17,20,23-octaoxahexacosan-26-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide,
(9H-Fluoren-9-yl)methyl {(26S)-34-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-20,27-dioxo-2,5,8,11,14,17-hexaoxa-21,28-diazatetratriacontan-26-yl}carbamate,
N-{4-[6-oxo-1-(6-{[N⁶-(20-oxo-2,5,8,11,14,17-hexaoxaicosan-20-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide,
(9H-Fluoren-9-yl)methyl {(20S)-28-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]-14,21-dioxo-2,5,8,11-tetraoxa-15,22-diazaoctacosan-20-yl}carbamate,
N-{4-[6-oxo-1-(6-{[N⁶-(14-oxo-2,5,8,11-tetraoxatetradecan-14-yl)-L-lysyl]amino}hexyl)-5-(quinolin-5-yl)-1,4,5,6-tetrahydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]hexyl}carbamate,
N-{4-[1-(6-Aminohexyl)-6-oxo-5-(quinolin-7-yl)-1,6-dihydropyridazin-3-yl]phenyl}-1,3-dihydro-2H-pyrrolo[3,4-c]pyridine-2-carboxamide,
N-{4-[1-(6-Aminohexyl)-6-oxo-5-(quinolin-7-yl)-1,6-dihydropyridazin-3-yl]phenyl}-1,3-dihydro-2H-pyrrolo[3,4-c]pyridine-2-carboxamide-hydrogen chloride
(9H-Fluoren-9-yl)methyl {(26S)-34-[3-{4-[(1,3-dihydro-2H-pyrrolo[3,4-c]pyridine-2-carbonyl)amino]phenyl}-6-oxo-5-(quinolin-7-yl)pyridazin-1(6H)-yl]-20,27-dioxo-2,5,8,11,14,17-hexaoxa-21,28-diazatetratriacontan-26-yl}carbamate,
N-{4-[6-oxo-1-(6-{[N⁶-(20-oxo-2,5,8,11,14,17-hexaoxaicosan-20-yl)-L-lysyl]amino}hexyl)-5-(quinolin-7-yl)-1,6-dihydropyridazin-3-yl]phenyl}-1,3-dihydro-2H-pyrrolo[3,4-c]pyridine-2-carboxamide,
tert-Butyl {6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}carbamate,
N-{4-[1-(6-Aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
N-{4-[1-(6-Aminohexyl)-6-oxo-5-(quinolin-7-yl)-1,4,5,6-tetrahydropyridazin-3-yl]phenyl}-1,3-dihydro-2H-pyrrolo[3,4-c]pyridine-2-carboxamide hydrogen chloride,
tert-Butyl N²-(tert-butoxycarbonyl)-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparaginate,
N-{6-[3-{4-1[(1,3-Dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-D-asparagine,
tert-Butyl [(2S)-1-({6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}amino)-1-oxopropan-2-yl]carbamate,
N-{4-[1-[6-(L-Alanylamino)hexyl]-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
N-(tert-Butoxycarbonyl)-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide,
L-Valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide,
N-[6-(2,5-Dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide,
N-[6-(2,5-dioxo-2,5-dihydro-1H-pyrrol-1-yl)hexanoyl]-L-valyl-N-{4-[3-{4-[(1,3-dihydro-2H-pyrrolo[3,4-c]pyridine-2-carbonyl)amino]phenyl}-6-oxo-5-(quinolin-5-yl)-5,6-dihydropyridazin-1(4H)-yl]butyl}-L-alaninamide,
N-{4-[1-(6-{[6-(2,5-Dioxo-2,5-dihydro-1H-pyrrol-1-yl)hexanoyl]amino}hexyl)-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[1-{6-[2-(2,5-Dioxo-2,5-dihydro-1H-pyrrol-1-yl)acetamido]hexyl}-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,4,5,6-tetrahydropyridazin-3-yl]phenyl}-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-6-oxo-5-(quinolin-5-yl)-1,6-dihydropyridazin-3-yl]phenyl}-1,3-dihydro-2H-pyrrolo[3,4-c]pyridine-2-carboxamide,
N-{4-[1-(4-{[6-(2,5-dioxo-2,5-dihydro-1H-pyrrol-1-yl)hexanoyl]amino}butyl)-6-oxo-5-(quinolin-7-yl)-1,6-dihydropyridazin-3-yl]phenyl}-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-5-(5-methyl-1,3,4-oxadiazol-2-yl)-6-oxo-1,6-dihydropyridazin-3-yl]phenyl}-1,3-dihydro-2H-pyrrolo[3,4-c]pyridine-2-carboxamide,
N-(4-{1-[6-({N²-[(2,5-Dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,6-dihydropyridazin-3-yl}phenyl)-1,3-dihydro-2H-pyrrolo[3,4-c]pyridine-2-carboxamide,
N-{4-[1-[6-({N²-[(2,5-dioxo-2,5-dihydro-1H-pyrrol-1-yl)acetyl]-N⁶-(26-oxo-2,5,8,11,14,17,20,23-octaoxahexacosan-26-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[1-[6-({N²-[(2,5-Dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{4-[1-[6-({N²-[(2,5-dioxo-2,5-dihydro-1H-pyrrol-1-yl)acetyl]-N⁶-(14-oxo-2,5,8,11-tetraoxatetradecan-14-yl)-L-lysyl}amino)hexyl]-6-oxo-5-(quinolin-5-yl)-1,4,5,6-tetrahydropyridazin-3-yl]phenyl}-1,3-dihydro-2H-pyrrolo[3,4-c]pyridine-2-carboxamide,
N-{5-[(2,5-Dioxopyrrolidin-1-yl)oxy]-5-oxopentanoyl}-L-valyl-N-{4-[3-{4-[(1,3-dihydro-2H-pyrrolo[3,4-c]pyridine-2-carbonyl)amino]phenyl}-6-oxo-5-(quinolin-5-yl)pyridazin-1(6H)-yl]butyl}-L-alaninamide,
N-(4-{1-[6-({N²-[(2,5-dioxo-2,5-dihydro-1H-pyrrol-1-yl)acetyl]-N⁶-(20-oxo-2,5,8,11,14,17-hexaoxaicosan-20-yl)-L-lysyl}amino)hexyl]-6-oxo-5-(quinolin-7-yl)-1,6-dihydropyridazin-3-yl}phenyl)-1,3-dihydro-2H-pyrrolo[3,4-c]pyridine-2-carboxamide,
N-{4-[1-{6-[2-(2,5-dioxo-2,5-dihydro-1H-pyrrol-1-yl)acetamido]hexyl}-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
N-{6-[3-{4-[(1,3-Dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-N²-[(2,5-dioxo-2,5-dihydro-1H-pyrrol-1-yl)acetyl]-D-asparagine, and
N-[(2,5-dioxo-2,5-dihydro-1H-pyrrol-1-yl)acetyl]-L-valyl-N-{6-[3-{4-[(1,3-dihydro-2H-pyrrolo[3,4-c]pyridine-2-carbonyl)amino]phenyl}-6-oxo-5-(quinolin-7-yl)-5,6-dihydropyridazin-1(4H)-yl]hexyl}-L-alaninamide,
or an N-oxide, a salt, a tautomer or a stereoisomer of said compound, or a salt of said N-oxide, tautomer or stereoisomer.

41: A compound selected from: 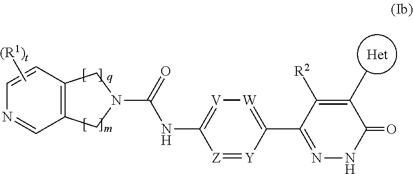
wherein
Het represents a heteroaryl group optionally substituted with one or more groups independently selected from R⁵;
R¹ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷ or —NH₂;
t is 0, 1 or 2;
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷;
R⁵ represents, independently of each other, halogen, hydroxy, C₁-C₃-alkyl, C₁-C₃-haloalkyl, C₁-C₃-alkoxy, C₁-C₃-haloalkoxy, —N(H)R⁶, —N(R⁶)R⁷—NH₂, 4- to 7-membered heterocycloalkyl, —SR⁸, —S(═O)R⁸, —S(═O)₂R⁸ or —S(═O)(═NR⁸)R⁹;
g is 0, 1, 2 or 3,
m is 0, 1, 2 or 3,
with the proviso that q+m is 2, 3 or 4; 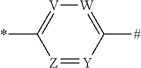
represents a group which is selected from: 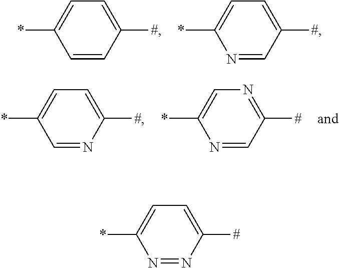
in which * and ^{#} represent the points of attachment of said group with the rest of the compound of formula (I),
said group being optionally substituted with one or more substituents independently selected from the group consisting of:
halogen, C₁-C₃-alkyl, C₁-C₃-alkoxy, C₁-C₃-haloalkoxy, R⁶(H)N— and —N(R⁶)R⁷;
R⁶, R⁷ represent, independently of each other, C₁-C₃-alkyl, C₃-C₆-cycloalkyl, phenyl, —C(═O)—O—(C₁-C₄-alkyl) or —C(═O)—(C₁-C₃-alkyl),
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷;
R⁸, R⁹ represent, independently of each other, hydrogen, C₁-C₃-alkyl, C₃-C₆-cycloalkyl, phenyl or C₁-C₃-haloalkyl,
wherein said phenyl is optionally substituted with one or more substituents independently selected from the group consisting of:
halogen, C₁-C₃-alkyl, C₁-C₃-alkoxy, C₁-C₃-haloalkoxy, —NH₂, —N(H)R⁶, and —N(R⁶)R⁷; 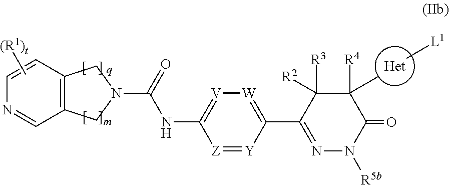
R¹, Het, t, q, m, V, W, Z and Y are as defined above, and
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷;
R³ represents H, C₁-C₃-alkyl or C₁-C₃-haloalkyl; and
R⁴ represents H, C₁-C₆-alkyl, C₃-C₆-cycloalkyl, C₁-C₄-haloalkyl or phenyl;
or,
R² and R³ together with the carbon to which they are attached form a C₃-C₆-cycloalkyl group or a 5- to 7-membered heterocycloalkyl group containing one heteroatom containing group selected from O, NR⁸, S, S(═O), S(═O)₂, S(═NR⁸)(═NR⁹) and S(═O)(═NR⁸); and
R⁴ represents H, C₁-C₆-alkyl, C₃-C₆-cycloalkyl, C₁-C₄-haloalkyl or phenyl;
or,
R² represents H, C₁-C₆-alkyl, C₃-C₆-cycloalkyl, C₁-C₄-haloalkyl or phenyl,
wherein phenyl is optionally substituted with one or more substituents independently selected from the group consisting of:
halogen, C₁-C₃-alkyl, C₁-C₃-alkoxy, C₁-C₃-haloalkoxy, —N(H)R⁶, and —N(R⁶)R⁷; and
R³ and R⁴ together form a bond;
R^{5b} represents hydrogen or a group selected from:
methyl, C₂-C₆-alkyl, (1,3-dioxolan-2-yl)-(C₁-C₆-alkyl)-, (1,3-dioxan-2-yl)-(C₁-C₆-alkyl)-, azetidin-3-yl, (azetidin-3-yl)-(C₁-C₆-alkyl)-, oxetan-3-yl, (oxetan-3-yl)-(C₁-C₆-alkyl)-, C₃-C₆-cycloalkyl, (C₃-C₆-cycloalkyl)-(C₁-C₆-alkyl)-, a 5- to 7-membered heterocycloalkyl group, (5- to 7-membered heterocycloalkyl)-(C₁-C₆-alkyl)-, phenyl, phenyl-(C₁-C₆-alkyl)-, a 5- to 6-membered heteroaryl group and (5- to 6-membered heteroaryl)-(C₁-C₆-alkyl)-, in which 5- to 7-membered heterocycloalkyl and 5- to 6-membered heteroaryl are connected to the rest of the molecule via a carbon atom of the 5- to 7-membered heterocycloalkyl ring or via a carbon atom of the 5- to 6-membered heteroaryl ring, respectively;
wherein C₂-C₆-alkyl is optionally substituted with one or more substituents independently selected from the group consisting of:
halogen, hydroxy, C₁-C₃-alkoxy, C₁-C₃-haloalkoxy, oxo (═O), —NH₂, —N(H)R⁶, —N(R⁶)R⁷, —C(═O)OR⁸, —SR⁸, —S(═O)R⁸, —S(═O)₂R⁸ and —S(═O)(═NR⁸)R⁹;
wherein azetidin-3-yl and oxetan-3-yl are optionally substituted with one or two substituents independently selected from the group consisting of:
C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, and C₃-C₆-cycloalkyloxy;
wherein C₃-C₆-cycloalkyl and 5- to 7-membered heterocycloalkyl are optionally substituted with one or more substituents independently selected from the group consisting of:
hydroxy, halogen, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-, C₃-C₆-cycloalkyl, C₃-C₆-cycloalkyloxy, —N(R⁵)R⁶, —C(═O)OH, oxo (═O), and —N(H)C(═O)—(C₁-C₃-alkyl);
wherein phenyl and 5- to 6-membered heteroaryl are optionally substituted with one or more substituents independently selected from the group consisting of:
halogen, C₁-C₃-alkyl, C₁-C₃-alkoxy-, C₁-C₃-haloalkoxy-, —N(H)R⁶, —N(R⁶)R⁷, —C(═O)OH and —C(═O)O(C₁-C₆-alkyl);
L1 is an in vivo non-cleavable organic group: 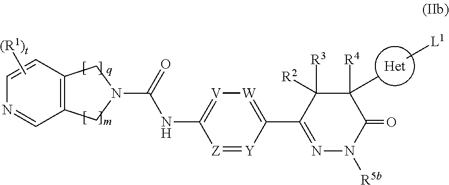
R¹, R², R³, R⁴, R^{5b}, Het, L¹, t, q, m, V, W, Z and Y are as defined above; 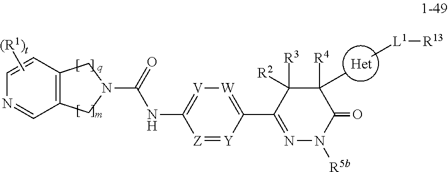
wherein R¹, R², R³, R⁴, R^{5b}, Het, L¹, t, q, m, V, W, Z and Y are as defined above, R¹³ represents —NHPG1, and PG¹ represents an amine protecting group; 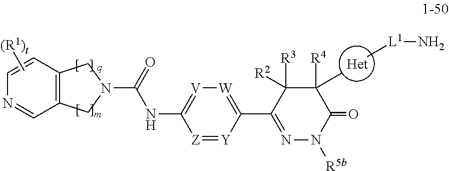
wherein R¹, R², R³, R⁴, Het, L¹, t, q, m, V, W, Z and Y are as defined above; 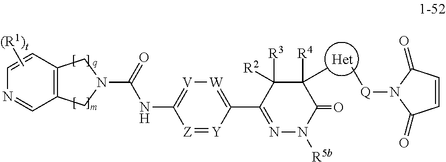
wherein R¹, R², R³, R⁴, R^{5b}, Het, t, q, m, V, W, Z and Y are as defined above and Q represents one of the following general structures (i) to (iii):
(i) §-L1-SG-§§
(ii) §-L1-SG-L1′-§§
(iii) §-L1-§§
wherein
§ represents the attachment point to ring Het;
§§ represents the attachment point to the maleimide group;
SG represents an in vivo cleavable group, L1 and L1′ represent, independently of each other, an in vivo non-cleavable organic group; 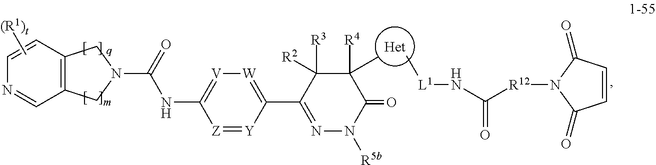
wherein R¹, R², R³, R⁴, R^{5b}, Het, L¹, t, q, m, V, W, Z and Y are as defined above, and R¹² is C₁-C₁₀-alkyl; 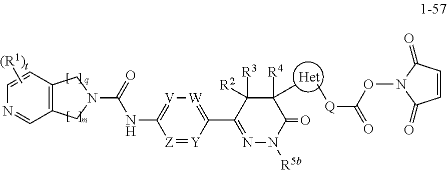
wherein R¹, R², R³, R⁴, R^{5b}, Het, t, q, m, V, W, Z and Y are as defined above and Q represents one of the following general structures (i) to (iii):
(i) §-L1-SG-§§
(ii) §-L1-SG-L1′-§§
(iii) §-L1-§§
wherein
§ represents the attachment point to ring Het;
§§ represents the attachment point to the carbonyl group;
SG represents an in vivo cleavable group, L1 and L1′ represent, independently of each other, an in vivo non-cleavable organic group; and 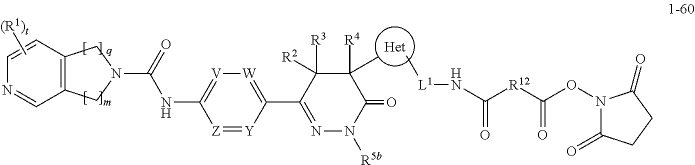
wherein R¹, R², R³, R⁴, R^{5b}, Het, L¹, t, q, m, V, W, Z and Y are as defined above and wherein R¹² is C₁-C₁₀ alkyl,
or an N-oxide, a salt, a tautomer or a stereoisomer of said compound, or a salt of said N-oxide, tautomer or stereoisomer.

42: The compound according to claim 41, which is selected from the group consisting of:
tert-Butyl (6-{5-[6-{4-[(1,3-dihydro-2H-pyrrolo[3,4-c]pyridine-2-carbonyl)amino]phenyl}-3-oxo-2-(2,2,2-trifluoroethyl)-2,3-dihydropyridazin-4-yl]-1H-indazol-1-yl}hexyl)carbamate,
N-(4-{5-[1-(6-Aminohexyl)-1H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
N-(4-{5-[2-(6-aminohexyl)-2H-indazol-5-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
tert-Butyl (6-{4-[6-{4-[(1,3-dihydro-2H-pyrrolo[3,4-c]pyridin-2-ylcarbonyl)amino]phenyl}-3-oxo-2-(2,2,2-trifluoroethyl)-2,3-dihydropyridazin-4-yl]-1H-indazol-1-yl}hexyl)carbamate,
N-(4-{5-[1-(6-Aminohexyl)-1H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
N-(4-{5-[2-(6-Aminohexyl)-2H-indazol-4-yl]-6-oxo-1-(2,2,2-trifluoroethyl)-1,6-dihydropyridazin-3-yl}phenyl)-1,3-dihydro-2H-pyrrolo[3,4-c]pyridine-2-carboxamide,
N-{4-[5-(1-{6-[2-(2,5-dioxo-2,5-dihydro-1H-pyrrol-1-yl)acetamido]hexyl}-1H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(2-{6-[2-(2,5-dioxo-2,5-dihydro-1H-pyrrol-1-yl)acetamido]hexyl}-2H-indazol-5-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(1-{6-[2-(2,5-dioxo-2,5-dihydro-1H-pyrrol-1-yl)acetamido]hexyl}-1H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide,
N-{4-[5-(2-{6-[2-(2,5-dioxo-2,5-dihydro-1H-pyrrol-1-yl)acetamido]hexyl}-2H-indazol-4-yl)-6-oxo-1-(2,2,2-trifluoroethyl)-1,6-dihydropyridazin-3-yl]phenyl}-1,3-dihydro-2H-pyrrolo[3,4-c]pyridine-2-carboxamide, and
N-{4-[1-{6-[2-(2,5-dioxo-2,5-dihydro-1H-pyrrol-1-yl)acetamido]hexyl}-6-oxo-5-(quinolin-7-yl)-1,4,5,6-tetrahydropyridazin-3-yl]phenyl}-1,3-dihydro-2H-pyrrolo[3,4-c]pyridine-2-carboxamide,
or an N-oxide, a salt, a tautomer or a stereoisomer of said compound, or a salt of said N-oxide, tautomer or stereoisomer.

43. (canceled)

44: A method of preparing the conjugate of claim 1, according to either of the following reaction schemes: 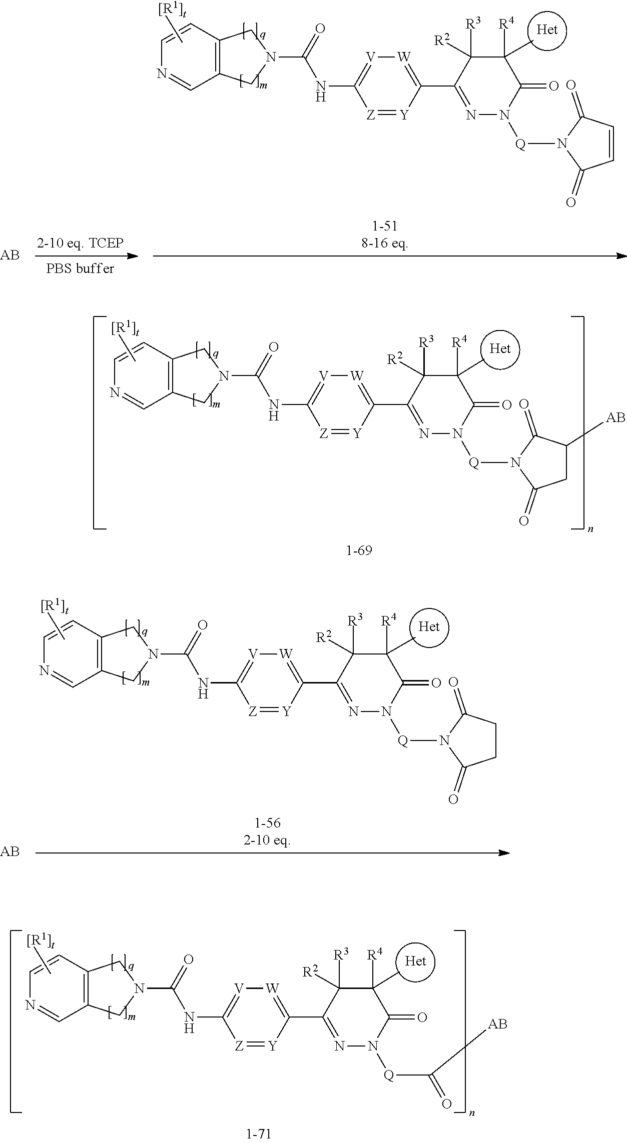
wherein AB, n, R¹, R², R³, R⁴, R⁵, L¹, L1′, t, q, m, V, W, Z and Y are defined as claim 1, Q represents one of the following general structures (i) to (iii):
(i) §-L1-SG-§§
(ii) §-L1-SG-L1′-§§
(iii) §-L1-§§
wherein
§ represents the attachment point to the pyridazinone ring;
§§ represents the attachment point to the maleimide (compound of formula 1-51) or succinimide (compound of formula 1-69) group, or to the carbonyl group (compounds of formula 1-56 and 1-71);
SG represents an in vivo cleavable group, L1 and L1′ represent, independently of each other, an in vivo non-cleavable organic group.

45: A method of preparing the conjugate of claim 1, according to either of the following reaction schemes: 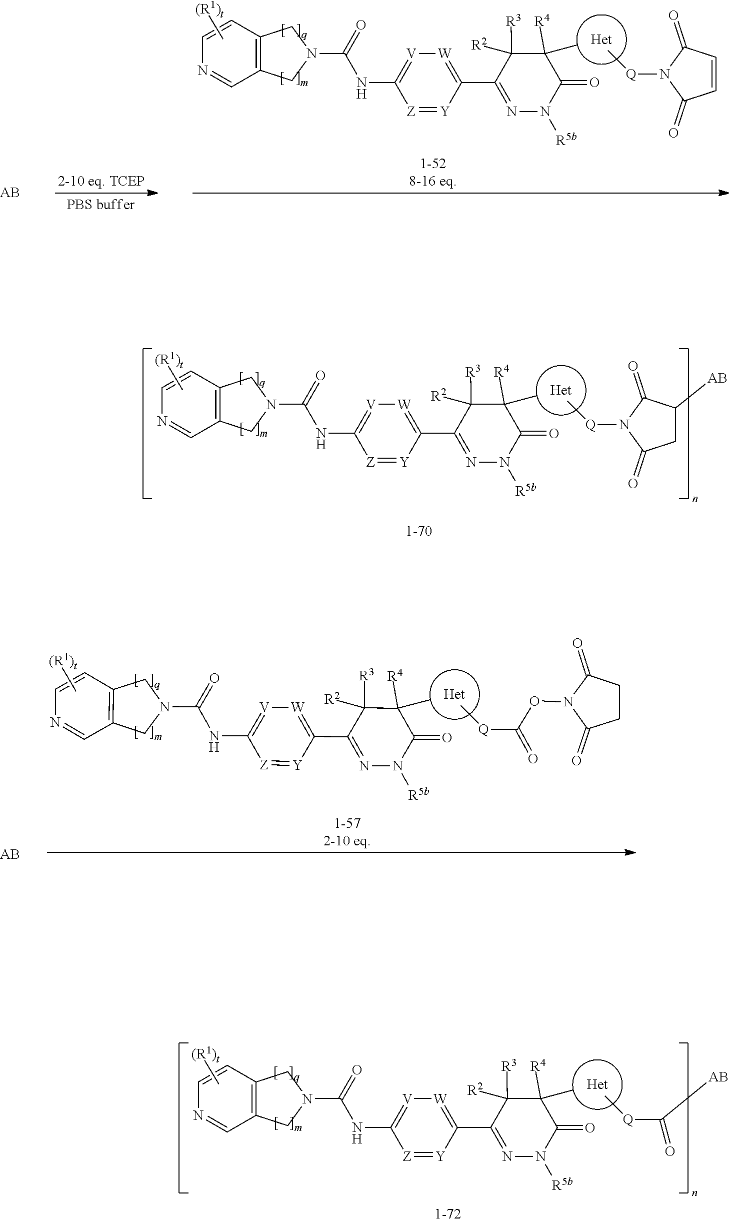
wherein AB, n, R¹, R², R³, R⁴, R⁵, t, q, m, V, W, Z and Y are defined claim 1, Q represents one of the following general structures (i) to (iii):
(i) §-L1-SG-§§
(ii) §-L1-SG-L1′-§§
(iii) §-L1-§§
wherein
§ represents the attachment point to Het;
§§ represents the attachment point to the maleimide (compound of formula 1-52) or succinimide (compound of formula 1-70) group, or to the carbonyl group (compounds of formula 1-57 and 1-72);
SG represents an in vivo cleavable group, L1 and L1′ represent, independently of each other, an in vivo non-cleavable organic group.

46. A method for the treatment or prophylaxis of a disease in a subject in need thereof, comprising administering an effective amount of a conjugate according to claim 1 to the subject.

47: The method of claim 46, wherein the disease is a hyperproliferative disease and/or a disorder responsive to induction of cell death.

48: The method of claim 47, wherein the hyperproliferative disease and/or disorder responsive to induction of cell death is a haematological tumour, solid tumour and/or metastases thereof.

49: The method of claim 48, wherein the hyperproliferative disease and/or disorder is a cancer disease.

50: The method of claim 49, wherein said cancer is selected from the group consisting of acute myeloid leukemia (AML), non-Hodgkin's lymphoma, breast cancer, brain tumors and lung cancer, and metastases thereof.

51: The method of claim 46, wherein the disease is a tumour or cancer, wherein the tumor or cancer is deficient in nicotinic acid pathway.

52: A pharmaceutical composition comprising a conjugate according to claim 1, together with at least one pharmaceutically acceptable carrier or auxiliary.

53: A method for treatment of a haematological tumour, a solid tumour and/or metastases thereof in a subject in need thereof, comprising administering a composition according to claim 52 to the subject.

54: A pharmaceutical combination comprising one or more first active ingredients selected from a conjugate according to claim 1, and:
a) one or more second active ingredients selected from chemotherapeutic anti-cancer agents and target-specific anti-cancer agents;
b) radiation therapy; and/or
c) a method or an agent which causes or induces DNA damage.
